SEQUENCE LISTING (33 sequences)

SEQ ID NO: 1 - DNA, 11055 bp, Homo sapiens
  source 1..11055: mol_type = other DNA
atgctttggtgggaagaagtagaggactgttatgaaagagaagatgttcaaaagaaaacattcacaaaatgggtaaatgcacaattttctaagtttgggaagcagcatattgagaacctcttcagtgacctacaggatgggaggcgcctcctagacctcctcgaaggcctgacagggcaaaaactgccaaaagaaaaaggatccacaagagttcatgccctgaacaatgtcaacaaggcactgcgggttttgcagaacaataatgttgatttagtgaatattggaagtactgacatcgtagatggaaatcataaactgactcttggtttgatttggaatataatcctccactggcaggtcaaaaatgtaatgaaaaatatcatggctggattgcaacaaaccaacagtgaaaagattctcctgagctgggtccgacaatcaactcgtaattatccacaggttaatgtaatcaacttcaccaccagctggtctgatggcctggctttgaatgctctcatccatagtcataggccagacctatttgactggaatagtgtggtttgccagcagtcagccacacaacgactggaacatgcattcaacatcgccagatatcaattaggcatagagaaactactcgatcctgaagatgttgataccacctatccagataagaagtccatcttaatgtacatcacatcactcttccaagttttgcctcaacaagtgagcattgaagccatccaggaagtggaaatgttgccaaggccacctaaagtgactaaagaagaacattttcagttacatcatcaaatgcactattctcaacagatcacggtcagtctagcacagggatatgagagaacttcttcccctaagcctcgattcaagagctatgcctacacacaggctgcttatgtcaccacctctgaccctacacggagcccatttccttcacagcatttggaagctcctgaagacaagtcatttggcagttcattgatggagagtgaagtaaacctggaccgttatcaaacagctttagaagaagtattatcgtggcttctttctgctgaggacacattgcaagcacaaggagagatttctaatgatgtggaagtggtgaaagaccagtttcatactcatgaggggtacatgatggatttgacagcccatcagggccgggttggtaatattctacaattgggaagtaagctgattggaacaggaaaattatcagaagatgaagaaactgaagtacaagagcagatgaatctcctaaattcaagatgggaatgcctcagggtagctagcatggaaaaacaaagcaatttacatagagttttaatggatctccagaatcagaaactgaaagagttgaatgactggctaacaaaaacagaagaaagaacaaggaaaatggaggaagagcctcttggacctgatcttgaagacctaaaacgccaagtacaacaacataaggtgcttcaagaagatctagaacaagaacaagtcagggtcaattctctcactcacatggtggtggtagttgatgaatctagtggagatcacgcaactgctgctttggaagaacaacttaaggtattgggagatcgatgggcaaacatctgtagatggacagaagaccgctgggttcttttacaagacatccttctcaaatggcaacgtcttactgaagaacagtgcctttttagtgcatggctttcagaaaaagaagatgcagtgaacaagattcacacaactggctttaaagatcaaaatgaaatgttatcaagtcttcaaaaactggccgttttaaaagcggatctagaaaagaaaaagcaatccatgggcaaactgtattcactcaaacaagatcttctttcaacactgaagaataagtcagtgacccagaagacggaagcatggctggataactttgcccggtgttgggataatttagtccaaaaacttgaaaagagtacagcacagatttcacaggctgtcaccaccactcagccatcactaacacagacaactgtaatggaaacagtaactacggtgaccacaagggaacagatcctggtaaagcatgctcaagaggaacttccaccaccacctccccaaaagaagaggcagattactgtggattctgaaattaggaaaaggttggatgttgatataactgaacttcacagctggattactcgctcagaagctgtgttgcagagtcctgaatttgcaatctttcggaaggaaggcaacttctcagacttaaaagaaaaagtcaatgccatagagcgagaaaaagctgagaagttcagaaaactgcaagatgccagcagatcagctcaggccctggtggaacagatggtgaatgagggtgttaatgcagatagcatcaaacaagcctcagaacaactgaacagccggtggatcgaattctgccagttgctaagtgagagacttaactggctggagtatcagaacaacatcatcgctttctataatcagctacaacaattggagcagatgacaactactgctgaaaactggttgaaaatccaacccaccaccccatcagagccaacagcaattaaaagtcagttaaaaatttgtaaggatgaagtcaaccggctatcagatcttcaacctcaaattgaacgattaaaaattcaaagcatagccctgaaagagaaaggacaaggacccatgttcctggatgcagactttgtggcctttacaaatcattttaagcaagtcttttctgatgtgcaggccagagagaaagagctacagacaatttttgacactttgccaccaatgcgctatcaggagaccatgagtgccatcaggacatgggtccagcagtcagaaaccaaactctccatacctcaacttagtgtcaccgactatgaaatcatggagcagagactcggggaattgcaggctttacaaagttctctgcaagagcaacaaagtggcctatactatctcagcaccactgtgaaagagatgtcgaagaaagcgccctctgaaattagccggaaatatcaatcagaatttgaagaaattgagggacgctggaagaagctctcctcccagctggttgagcattgtcaaaagctagaggagcaaatgaataaactccgaaaaattcagaatcacatacaaaccctgaagaaatggatggctgaagttgatgtttttctgaaggaggaatggcctgcccttggggattcagaaattctaaaaaagcagctgaaacagtgcagacttttagtcagtgatattcagacaattcagcccagtctaaacagtgtcaatgaaggtgggcagaagataaagaatgaagcagagccagagtttgcttcgagacttgagacagaactcaaagaacttaacactcagtgggatcacatgtgccaacaggtctatgccagaaaggaggccttgaagggaggtttggagaaaactgtaagcctccagaaagatctatcagagatgcacgaatggatgacacaagctgaagaagagtatcttgagagagattttgaatataaaactccagatgaattacagaaagcagttgaagagatgaagagagctaaagaagaggcccaacaaaaagaagcgaaagtgaaactccttactgagtctgtaaatagtgtcatagctcaagctccacctgtagcacaagaggccttaaaaaaggaacttgaaactctaaccaccaactaccagtggctctgcactaggctgaatgggaaatgcaagactttggaagaagtttgggcatgttggcatgagttattgtcatacttggagaaagcaaacaagtggctaaatgaagtagaatttaaacttaaaaccactgaaaacattcctggcggagctgaggaaatctctgaggtgctagattcacttgaaaatttgatgcgacattcagaggataacccaaatcagattcgcatattggcacagaccctaacagatggcggagtcatggatgagctaatcaatgaggaacttgagacatttaattctcgttggagggaactacatgaagaggctgtaaggaggcaaaagttgcttgaacagagcatccagtctgcccaggagactgaaaaatccttacacttaatccaggagtccctcacattcattgacaagcagttggcagcttatattgcagacaaggtggacgcagctcaaatgcctcaggaagcccagaaaatccaatctgatttgacaagtcatgagatcagtttagaagaaatgaagaaacataatcaggggaaggaggctgcccaaagagtcctgtctcagattgatgttgcacagaaaaaattacaagatgtctccatgaagtttcgattattccagaaaccagccaattttgagcagcgtctacaagaaagtaagatgattttagatgaagtgaagatgcacttgcctgcattggaaacaaagagtgtggaacaggaagtagtacagtcacagctaaatcattgtgtgaacttgtataaaagtctgagtgaagtgaagtctgaagtggaaatggtgataaagactggacgtcagattgtacagaaaaagcagacggaaaatcccaaagaacttgatgaaagagtaacagctttgaaattgcattataatgagctgggagcaaaggtaacagaaagaaagcaacagttggagaaatgcttgaaattgtcccgtaagatgcgaaaggaaatgaatgtcttgacagaatggctggcagctacagatatggaattgacaaagagatcagcagttgaaggaatgcctagtaatttggattctgaagttgcctggggaaaggctactcaaaaagagattgagaaacagaaggtgcacctgaagagtatcacagaggtaggagaggccttgaaaacagttttgggcaagaaggagacgttggtggaagataaactcagtcttctgaatagtaactggatagctgtcacctcccgagcagaagagtggttaaatcttttgttggaataccagaaacacatggaaacttttgaccagaatgtggaccacatcacaaagtggatcattcaggctgacacacttttggatgaatcagagaaaaagaaaccccagcaaaaagaagacgtgcttaagcgtttaaaggcagaactgaatgacatacgcccaaaggtggactctacacgtgaccaagcagcaaacttgatggcaaaccgcggtgaccactgcaggaaattagtagagccccaaatctcagagctcaaccatcgatttgcagccatttcacacagaattaagactggaaaggcctccattcctttgaaggaattggagcagtttaactcagatatacaaaaattgcttgaaccactggaggctgaaattcagcagggggtgaatctgaaagaggaagacttcaataaagatatgaatgaagacaatgagggtactgtaaaagaattgttgcaaagaggagacaacttacaacaaagaatcacagatgagagaaagcgagaggaaataaagataaaacagcagctgttacagacaaaacataatgctctcaaggatttgaggtctcaaagaagaaaaaaggctctagaaatttctcatcagtggtatcagtacaagaggcaggctgatgatctcctgaaatgcttggatgacattgaaaaaaaattagccagcctacctgagcccagagatgaaaggaaaataaaggaaattgatcgggaattgcagaagaagaaagaggagctgaatgcagtgcgtaggcaagctgagggcttgtctgaggatggggccgcaatggcagtggagccaactcagatccagctcagcaagcgctggcgggaaattgagagcaaatttgctcagtttcgaagactcaactttgcacaaattcacactgtccgtgaagaaacgatgatggtgatgactgaagacatgcctttggaaatttcttatgtgccttctacttatttgactgaaatcactcatgtctcacaagccctattagaagtggaacaacttctcaatgctcctgacctctgtgctaaggactttgaagatctctttaagcaagaggagtctctgaagaatataaaagatagtctacaacaaagctcaggtcggattgacattattcatagcaagaagacagcagcattgcaaagtgcaacgcctgtggaaagggtgaagctacaggaagctctctcccagcttgatttccaatgggaaaaagttaacaaaatgtacaaggaccgacaagggcgatttgacagatctgttgagaaatggcggcgttttcattatgatataaagatatttaatcagtggctaacagaagctgaacagtttctcagaaagacacaaattcctgagaattgggaacatgctaaatacaaatggtatcttaaggaactccaggatggcattgggcagcggcaaactgttgtcagaacattgaatgcaactggggaagaaataattcagcaatcctcaaaaacagatgccagtattctacaggaaaaattgggaagcctgaatctgcggtggcaggaggtctgcaaacagctgtcagacagaaaaaagaggctagaagaacaaaagaatatcttgtcagaatttcaaagagatttaaatgaatttgttttatggttggaggaagcagataacattgctagtatcccacttgaacctggaaaagagcagcaactaaaagaaaagcttgagcaagtcaagttactggtggaagagttgcccctgcgccagggaattctcaaacaattaaatgaaactggaggacccgtgcttgtaagtgctcccataagcccagaagagcaagataaacttgaaaataagctcaagcagacaaatctccagtggataaaggtttccagagctttacctgagaaacaaggagaaattgaagctcaaataaaagaccttgggcagcttgaaaaaaagcttgaagaccttgaagagcagttaaatcatctgctgctgtggttatctcctattaggaatcagttggaaatttataaccaaccaaaccaagaaggaccatttgacgttaaggaaactgaaatagcagttcaagctaaacaaccggatgtggaagagattttgtctaaagggcagcatttgtacaaggaaaaaccagccactcagccagtgaagaggaagttagaagatctgagctctgagtggaaggcggtaaaccgtttacttcaagagctgagggcaaagcagcctgacctagctcctggactgaccactattggagcctctcctactcagactgttactctggtgacacaacctgtggttactaaggaaactgccatctccaaactagaaatgccatcttccttgatgttggaggtacctgctctggcagatttcaaccgggcttggacagaacttaccgactggctttctctgcttgatcaagttataaaatcacagagggtgatggtgggtgaccttgaggatatcaacgagatgatcatcaagcagaaggcaacaatgcaggatttggaacagaggcgtccccagttggaagaactcattaccgctgcccaaaatttgaaaaacaagaccagcaatcaagaggctagaacaatcattacggatcgaattgaaagaattcagaatcagtgggatgaagtacaagaacaccttcagaaccggaggcaacagttgaatgaaatgttaaaggattcaacacaatggctggaagctaaggaagaagctgagcaggtcttaggacaggccagagccaagcttgagtcatggaaggagggtccctatacagtagatgcaatccaaaagaaaatcacagaaaccaagcagttggccaaagacctccgccagtggcagacaaatgtagatgtggcaaatgacttggccctgaaacttctccgggattattctgcagatgataccagaaaagtccacatgataacagagaatatcaatgcctcttggagaagcattcataaaagggtgagtgagcgagaggctgctttggaagaaactcatagattactgcaacagttccccctggacctggaaaagtttcttgcctggcttacagaagctgaaacaactgccaatgtcctacaggatgctacccgtaaggaaaggctcctagaagactccaagggagtaaaagagctgatgaaacaatggcaagacctccaaggtgaaattgaagctcacacagatgtttatcacaacctggatgaaaacagccaaaaaatcctgagatccctggaaggttccgatgatgcagtcctgttacaaagacgtttggataacatgaacttcaagtggagtgaacttcggaaaaagtctctcaacattaggtcccatttggaagccagttctgaccagtggaagcgtctgcacctttctctgcaggaacttctggtgtggctacagctgaaagatgatgaattaagccggcaggcacctattggaggcgactttccagcagttcagaagcagaacgatgtacatagggccttcaagagggaattgaaaactaaagaacctgtaatcatgagtactcttgagactgtacgaatatttctgacagagcagcctttggaaggactagagaaactctaccaggagcccagagagctgcctcctgaggagagagcccagaatgtcactcggcttctacgaaagcaggctgaggaggtcaatactgagtgggaaaaattgaacctgcactccgctgactggcagagaaaaatagatgagacccttgaaagactccaggaacttcaagaggccacggatgagctggacctcaagctgcgccaagctgaggtgatcaagggatcctggcagcccgtgggcgatctcctcattgactctctccaagatcacctcgagaaagtcaaggcacttcgaggagaaattgcgcctctgaaagagaacgtgagccacgtcaatgaccttgctcgccagcttaccactttgggcattcagctctcaccgtataacctcagcactctggaagacctgaacaccagatggaagcttctgcaggtggccgtcgaggaccgagtcaggcagctgcatgaagcccacagggactttggtccagcatctcagcactttctttccacgtctgtccagggtccctgggagagagccatctcgccaaacaaagtgccctactatatcaaccacgagactcaaacaacttgctgggaccatcccaaaatgacagagctctaccagtctttagctgacctgaataatgtcagattctcagcttataggactgccatgaaactccgaagactgcagaaggccctttgcttggatctcttgagcctgtcagctgcatgtgatgccttggaccagcacaacctcaagcaaaatgaccagcccatggatatcctgcagattattaattgtttgaccactatttatgaccgcctggagcaagagcacaacaatttggtcaacgtccctctctgcgtggatatgtgtctgaactggctgctgaatgtttatgatacgggacgaacagggaggatccgtgtcctgtcttttaaaactggcatcatttccctgtgtaaagcacatttggaagacaagtacagataccttttcaagcaagtggcaagttcaacaggattttgtgaccagcgcaggctgggcctccttctgcatgattctatccaaattccaagacagttgggtgaagttgcatcctttgggggcagtaacattgagccaagtgtccggagctgcttccaatttgctaataataagccagagatcgaagcggccctcttcctagactggatgagactggaaccccagtccatggtgtggctgcccgtcctgcacagagtggctgctgcagaaactgccaagcatcaggccaaatgtaacatctgcaaagagtgtccaatcattggattcaggtacaggagtctaaagcactttaattatgacatctgccaaagctgctttttttctggtcgagttgcaaaaggccataaaatgcactatcccatggtggaatattgcactccgactacatcaggagaagatgttcgagactttgccaaggtactaaaaaacaaatttcgaaccaaaaggtattttgcgaagcatccccgaatgggctacctgccagtgcagactgtcttagagggggacaacatggaaactcccgttactctgatcaacttctggccagtagattctgcgcctgcctcgtcccctcagctttcacacgatgatactcattcacgcattgaacattatgctagcaggctagcagaaatggaaaacagcaatggatcttatctaaatgatagcatctctcctaatgagagcatagatgatgaacatttgttaatccagcattactgccaaagtttgaaccaggactcccccctgagccagcctcgtagtcctgcccagatcttgatttccttagagagtgaggaaagaggggagctagagagaatcctagcagatcttgaggaagaaaacaggaatctgcaagcagaatatgaccgtctaaagcagcagcacgaacataaaggcctgtccccactgccgtcccctcctgaaatgatgcccacctctccccagagtccccgggatgctgagctcattgctgaggccaagctactgcgtcaacacaaaggccgcctggaagccaggatgcaaatcctggaagaccacaataaacagctggagtcacagttacacaggctaaggcagctgctggagcaaccccaggcagaggccaaagtgaatggcacaacggtgtcctctccttctacctctctacagaggtccgacagcagtcagcctatgctgctccgagtggttggcagtcaaacttcggactccatgggtgaggaagatcttctcagtcctccccaggacacaagcacagggttagaggaggtgatggagcaactcaacaactccttccctagttcaagaggaagaaatacccctggaaagccaatgagagaggacacaatg

SEQ ID NO: 2 - protein, 3685 aa, Homo sapiens
MLWWEEVEDCYEREDVQKKTFTKWVNAQFSKFGKQHIENLFSDLQDGRRLLDLLEGLTGQKLPKEKGSTRVHALNNVNKALRVLQNNNVDLVNIGSTDIVDGNHKLTLGLIWNIILHWQVKNVMKNIMAGLQQTNSEKILLSWVRQSTRNYPQVNVINFTTSWSDGLALNALIHSHRPDLFDWNSVVCQQSATQRLEHAFNIARYQLGIEKLLDPEDVDTTYPDKKSILMYITSLFQVLPQQVSIEAIQEVEMLPRPPKVTKEEHFQLHHQMHYSQQITVSLAQGYERTSSPKPRFKSYAYTQAAYVTTSDPTRSPFPSQHLEAPEDKSFGSSLMESEVNLDRYQTALEEVLSWLLSAEDTLQAQGEISNDVEVVKDQFHTHEGYMMDLTAHQGRVGNILQLGSKLIGTGKLSEDEETEVQEQMNLLNSRWECLRVASMEKQSNLHRVLMDLQNQKLKELNDWLTKTEERTRKMEEEPLGPDLEDLKRQVQQHKVLQEDLEQEQVRVNSLTHMVVVVDESSGDHATAALEEQLKVLGDRWANICRWTEDRWVLLQDILLKWQRLTEEQCLFSAWLSEKEDAVNKIHTTGFKDQNEMLSSLQKLAVLKADLEKKKQSMGKLYSLKQDLLSTLKNKSVTQKTEAWLDNFARCWDNLVQKLEKSTAQISQAVTTTQPSLTQTTVMETVTTVTTREQILVKHAQEELPPPPPQKKRQITVDSEIRKRLDVDITELHSWITRSEAVLQSPEFAIFRKEGNFSDLKEKVNAIEREKAEKFRKLQDASRSAQALVEQMVNEGVNADSIKQASEQLNSRWIEFCQLLSERLNWLEYQNNIIAFYNQLQQLEQMTTTAENWLKIQPTTPSEPTAIKSQLKICKDEVNRLSDLQPQIERLKIQSIALKEKGQGPMFLDADFVAFTNHFKQVFSDVQAREKELQTIFDTLPPMRYQETMSAIRTWVQQSETKLSIPQLSVTDYEIMEQRLGELQALQSSLQEQQSGLYYLSTTVKEMSKKAPSEISRKYQSEFEEIEGRWKKLSSQLVEHCQKLEEQMNKLRKIQNHIQTLKKWMAEVDVFLKEEWPALGDSEILKKQLKQCRLLVSDIQTIQPSLNSVNEGGQKIKNEAEPEFASRLETELKELNTQWDHMCQQVYARKEALKGGLEKTVSLQKDLSEMHEWMTQAEEEYLERDFEYKTPDELQKAVEEMKRAKEEAQQKEAKVKLLTESVNSVIAQAPPVAQEALKKELETLTTNYQWLCTRLNGKCKTLEEVWACWHELLSYLEKANKWLNEVEFKLKTTENIPGGAEEISEVLDSLENLMRHSEDNPNQIRILAQTLTDGGVMDELINEELETFNSRWRELHEEAVRRQKLLEQSIQSAQETEKSLHLIQESLTFIDKQLAAYIADKVDAAQMPQEAQKIQSDLTSHEISLEEMKKHNQGKEAAQRVLSQIDVAQKKLQDVSMKFRLFQKPANFEQRLQESKMILDEVKMHLPALETKSVEQEVVQSQLNHCVNLYKSLSEVKSEVEMVIKTGRQIVQKKQTENPKELDERVTALKLHYNELGAKVTERKQQLEKCLKLSRKMRKEMNVLTEWLAATDMELTKRSAVEGMPSNLDSEVAWGKATQKEIEKQKVHLKSITEVGEALKTVLGKKETLVEDKLSLLNSNWIAVTSRAEEWLNLLLEYQKHMETFDQNVDHITKWIIQADTLLDESEKKKPQQKEDVLKRLKAELNDIRPKVDSTRDQAANLMANRGDHCRKLVEPQISELNHRFAAISHRIKTGKASIPLKELEQFNSDIQKLLEPLEAEIQQGVNLKEEDFNKDMNEDNEGTVKELLQRGDNLQQRITDERKREEIKIKQQLLQTKHNALKDLRSQRRKKALEISHQWYQYKRQADDLLKCLDDIEKKLASLPEPRDERKIKEIDRELQKKKEELNAVRRQAEGLSEDGAAMAVEPTQIQLSKRWREIESKFAQFRRLNFAQIHTVREETMMVMTEDMPLEISYVPSTYLTEITHVSQALLEVEQLLNAPDLCAKDFEDLFKQEESLKNIKDSLQQSSGRIDIIHSKKTAALQSATPVERVKLQEALSQLDFQWEKVNKMYKDRQGRFDRSVEKWRRFHYDIKIFNQWLTEAEQFLRKTQIPENWEHAKYKWYLKELQDGIGQRQTVVRTLNATGEEIIQQSSKTDASILQEKLGSLNLRWQEVCKQLSDRKKRLEEQKNILSEFQRDLNEFVLWLEEADNIASIPLEPGKEQQLKEKLEQVKLLVEELPLRQGILKQLNETGGPVLVSAPISPEEQDKLENKLKQTNLQWIKVSRALPEKQGEIEAQIKDLGQLEKKLEDLEEQLNHLLLWLSPIRNQLEIYNQPNQEGPFDVKETEIAVQAKQPDVEEILSKGQHLYKEKPATQPVKRKLEDLSSEWKAVNRLLQELRAKQPDLAPGLTTIGASPTQTVTLVTQPVVTKETAISKLEMPSSLMLEVPALADFNRAWTELTDWLSLLDQVIKSQRVMVGDLEDINEMIIKQKATMQDLEQRRPQLEELITAAQNLKNKTSNQEARTIITDRIERIQNQWDEVQEHLQNRRQQLNEMLKDSTQWLEAKEEAEQVLGQARAKLESWKEGPYTVDAIQKKITETKQLAKDLRQWQTNVDVANDLALKLLRDYSADDTRKVHMITENINASWRSIHKRVSEREAALEETHRLLQQFPLDLEKFLAWLTEAETTANVLQDATRKERLLEDSKGVKELMKQWQDLQGEIEAHTDVYHNLDENSQKILRSLEGSDDAVLLQRRLDNMNFKWSELRKKSLNIRSHLEASSDQWKRLHLSLQELLVWLQLKDDELSRQAPIGGDFPAVQKQNDVHRAFKRELKTKEPVIMSTLETVRIFLTEQPLEGLEKLYQEPRELPPEERAQNVTRLLRKQAEEVNTEWEKLNLHSADWQRKIDETLERLRELQEATDELDLKLRQAEVIKGSWQPVGDLLIDSLQDHLEKVKALRGEIAPLKENVSHVNDLARQLTTLGIQLSPYNLSTLEDLNTRWKLLQVAVEDRVRQLHEAHRDFGPASQHFLSTSVQGPWERAISPNKVPYYINHETQTTCWDHPKMTELYQSLADLNNVRFSAYRTAMKLRRLQKALCLDLLSLSAACDALDQHNLKQNDQPMDILQIINCLTTIYDRLEQEHNNLVNVPLCVDMCLNWLLNVYDTGRTGRIRVLSFKTGIISLCKAHLEDKYRYLFKQVASSTGFCDQRRLGLLLHDSIQIPRQLGEVASFGGSNIEPSVRSCFQFANNKPEIEAALFLDWMRLEPQSMVWLPVLHRVAAAETAKHQAKCNICKECPIIGFRYRSLKHFNYDICQSCFFSGRVAKGHKMHYPMVEYCTPTTSGEDVRDFAKVLKNKFRTKRYFAKHPRMGYLPVQTVLEGDNMETPVTLINFWPVDSAPASSPQLSHDDTHSRIEHYASRLAEMENSNGSYLNDSISPNESIDDEHLLIQHYCQSLNQDSPLSQPRSPAQILISLESEERGELERILADLEEENRNLQAEYDRLKQQHEHKGLSPLPSPPEMMPTSPQSPRDAELIAEAKLLRQHKGRLEARMQILEDHNKQLESQLHRLRQLLEQPQAEAKVNGTTVSSPSTSLQRSDSSQPMLLRVVGSQTSDSMGEEDLLSPPQDTSTGLEEVMEQLNNSFPSSRGRNTPGKPMREDTM

SEQ ID NO: 3 - DNA, 11055 bp, Homo sapiens
  source 1..11055: mol_type = other DNA
atgctttggtgggaagaagtcgaggactgctacgagcgcgaggacgtgcagaagaaaaccttcaccaaatgggtcaacgcccagttcagcaagttcggcaagcagcacatcgagaacctgttcagcgacctgcaggatggcagaaggctgctggatctgctggaaggcctgacaggacagaagctgcccaaagagaagggcagcacaagagtgcacgccctgaacaacgtgaacaaggccctgagagtgctgcagaacaacaacgtggacctggtcaacatcggcagcaccgacatcgtggacggcaaccacaaactgaccctgggcctgatctggaacatcatcctgcactggcaagtgaagaacgtgatgaagaacatcatggccggcctgcagcagaccaacagcgagaagattctgctgagctgggtccgacagagcacccggaattaccctcaagtgaacgtgatcaacttcaccacctcttggagcgacggactggccctgaatgccctgatccacagccacagacctgacctgttcgactggaacagcgtcgtgtgtcagcagagcgccacacagaggctggaacacgccttcaatatcgccagataccagctgggcatcgagaaactgctggaccccgaggatgtggacaccacctatcctgacaagaaatccatcctcatgtacatcaccagcctgttccaggtgctgccccagcaggtttccatcgaggccattcaagaggtcgagatgctgcccagacctcctaaagtgaccaaagaggaacacttccagctgcaccaccagatgcactactctcagcagatcaccgtgtctctggcccagggctacgagagaacaagcagccccaagcctcggttcaagagctacgcctatacacaggccgcctacgtgaccaccagcgatcctacaagaagcccatttcctagccagcacctcgaggcccctgaggataagagctttggcagcagcctgatggaaagcgaagtgaacctggaccgctaccagacagccctggaagaggttctgagctggctgctgtctgccgaggatacactgcaggctcagggcgagatcagcaacgacgtggaagtggtcaaggaccagtttcacacccacgagggctacatgatggacctgacagcccaccagggcagagtgggcaatattctgcagctgggctccaagctgatcggcacaggcaagctgagcgaggacgaagagacagaggtgcaagagcagatgaacctgctgaacagcagatgggagtgtctgagagtggccagcatggaaaagcagagcaacctgcaccgggtgctgatggatctccagaaccagaagctgaaagagctgaacgactggctgaccaagaccgaggaacggacccggaagatggaagaggaacctctgggacccgacctggaagatctgaaaagacaggtgcagcagcataaggtgctgcaagaggacctcgagcaagagcaagtgcgcgtgaacagcctgacacacatggtggtggtcgtggatgagagcagcggagatcatgccacagccgctctggaagaacagctgaaggtgctgggagacagatgggccaatatctgccggtggaccgaggatagatgggtgctgctccaggacatcctgctgaagtggcagcggctgacagaggaacagtgcctgtttagcgcctggctgtccgagaaagaggacgccgtcaacaagatccacaccaccggcttcaaggatcagaatgagatgctgagcagcctgcagaaactggccgtgctgaaggctgacctggaaaagaaaaagcagtccatgggcaagctgtactccctgaagcaggacctgctgagcacactgaagaacaagagcgtgacccagaaaaccgaggcctggctggacaacttcgccagatgctgggacaacctggtgcagaagctggaaaagtccaccgctcagatctctcaggccgtgaccacaacacagccttctctgacccagaccaccgtgatggaaacagtgaccacagtgacaacccgcgagcagatcctggtcaagcacgcccaagaagaactgcctcctccacctcctcagaagaaacggcagatcacagtggacagcgagatccggaagagactggacgtggacatcaccgagctgcacagctggatcaccagatccgaagccgtgctgcagtcccctgagttcgccatcttcagaaaagagggcaacttctccgacctgaaagagaaagtcaacgccatcgagagagagaaggccgagaagttccggaagctgcaggacgcctccagatctgcccaggctctggtggaacagatggtcaacgaaggcgtgaacgccgacagcatcaagcaggcctcagagcagctgaactcccggtggatcgagttctgtcagctcctgagcgagagactgaactggctggaataccagaacaatatcattgccttctacaaccagctccagcagctcgaacagatgaccaccacagccgagaattggctgaagatccagcctaccacacctagcgagcccaccgccattaagagccagctgaaaatctgcaaggacgaagtgaatcggctgagcgatctgcagccccagatcgaaagactgaaaatccagtctatcgccctcaaagagaaaggacagggccccatgttcctggacgccgattttgtggcctttaccaaccacttcaaacaggtgttctccgacgtgcaggcccgggaaaaagagctgcagaccatcttcgacaccctgcctccaatgagataccaagagacaatgagcgccatccggacctgggtgcagcaaagcgagacaaagctgagcatcccacagctgtccgtgaccgactacgagatcatggaacagagactgggcgaactgcaggccctccagtctagcctgcaagaacagcagtccggcctgtactacctgagcacaaccgtgaaagagatgagcaagaaggcccctagcgagatctcccggaagtaccagagcgagttcgaagagatcgaaggccggtggaagaagctgtctagccagctggttgagcactgccagaaactcgaggaacaaatgaacaagctgcgcaagatccagaatcacatccagacgctgaagaaatggatggccgaggtggacgtgttcctgaaagaagagtggcccgctctgggcgactccgagattctgaagaaacagctcaaacagtgccggctgctggtgtccgatatccagacaatccagccaagcctgaactccgtgaatgaaggcggccagaagatcaagaacgaggccgagcctgagtttgccagcagactggaaaccgaactgaaagaactcaacacccagtgggaccacatgtgccagcaagtgtacgcccggaaagaggccctgaaaggcggactggaaaaaacagtgtctctgcagaaagacctcagcgagatgcacgagtggatgacccaggccgaagaggaatacctggaacgggacttcgagtacaagacccctgatgagctgcaaaaagccgtcgaggaaatgaagcgggccaaagaagaggcccagcagaaagaagccaaagtcaagctgctgaccgagtccgtgaatagcgttatcgctcaggcccctcctgtggctcaagaagctctgaaaaaagaactcgagacactgaccaccaactaccagtggctgtgcaccagactgaacggcaagtgcaagacactggaagaagtgtgggcctgctggcacgaactgctgtcctatctggaaaaggccaacaagtggctgaacgaggtggaattcaagctgaaaaccaccgagaacatccctggcggcgctgaagagatcagcgaagtgctggacagcctggaaaacctgatgagacacagcgaggacaaccccaatcagatcagaatcctggctcagaccctgaccgatggcggcgtgatggacgagctgatcaacgaggaactggaaacattcaacagccggtggcgggaactgcacgaggaagctgttcggagacagaaactcctcgagcagtctatccagagcgcacaagaaaccgagaagtccctgcacctgatccaagagagcctgaccttcatcgacaagcagctggccgcttatatcgccgacaaggtggacgctgctcagatgcctcaagaggctcagaagattcagagcgacctgaccagccacgagattagcctggaagaaatgaagaagcacaaccagggcaaagaggccgctcagagagtcctgagccagatcgatgtggcccagaaaaaactccaggacgtgtccatgaagtttcggctgtttcagaagcccgccaacttcgagcagagactgcaagagtccaagatgatcctggacgaagtcaaaatgcatctgcccgctctcgagactaagagcgtggaacaagaagtggtgcagtcccagctgaaccactgcgtgaacctgtacaagagcctgtccgaagtgaagtctgaggtggaaatggtcatcaagaccggcaggcagatcgtccagaagaagcagaccgagaatcccaaagaactggacgagagagtgaccgctctgaagctgcactacaatgagctgggcgccaaagtgaccgagcggaagcagcaactcgaaaagtgcctgaagctgagccgcaagatgcggaaagaaatgaacgtgctgacagagtggctggccgccaccgatatggaactgactaagagaagcgccgtggaaggcatgcccagcaacctggatagtgaagtggcctggggcaaagccactcagaaagagattgagaagcagaaggtccacctgaagtccatcaccgaagtgggcgaagccctgaaaaccgtgctgggcaagaaagaaaccctggtcgaggacaagctgtccctgctgaattccaactggatcgccgtgacctccagagctgaggaatggctgaatctgctgctcgagtaccagaaacacatggaaactttcgaccagaacgtcgaccacatcacaaagtggatcatccaggctgataccctgctggacgagtccgagaagaagaaaccccaacaaaaagaagatgtgctgaagcggctgaaagccgagctgaatgacatccggcctaaggtggacagcaccagagatcaggcagccaacctgatggccaacagaggcgaccactgtcggaagctggtcgagcctcagattagcgaactgaaccacagatttgccgccatcagccaccggatcaagacaggcaaggccagcattcctctcaaagagcttgagcagttcaacagcgacatccaaaagctgctcgaacccctggaagccgagatccaacagggcgtcaacctcaaagaagaagatttcaacaaggacatgaacgaggacaatgagggcaccgtcaaagaattgctgcagcggggcgacaatctgcagcagcggattaccgatgagcgcaagagagaggaaatcaagatcaagcagcagctcctgcagacaaagcacaacgcactgaaggacctgagaagccagcggcggaagaaggcactggaaatctcccaccagtggtatcagtacaagcggcaggccgacgacctgctcaagtgcctggacgacatcgaaaaaaagctggcctctctgcccgagcctagggacgagcggaaaatcaaagaaatcgaccgggaactccagaaaaagaaagaggaactgaacgccgtcagaaggcaggctgagggccttagtgaagatggggccgctatggccgtggaacccacacagatccaactgtccaagcgctggcgggaaatcgagagcaagtttgcccagttccggcggctgaatttcgcccagattcacaccgtgcgcgaagaaaccatgatggttatgaccgaggacatgcccctggaaatcagctacgtgccctctacctacctgaccgagatcacccatgtgtcacaggccctgcttgaggtcgaacagctgctgaacgcccctgatctgtgcgccaaggatttcgaggatctgttcaagcaagaggaatccctcaagaatatcaaggacagcctccagcaaagcagcggccggatcgatatcatccacagcaagaaaaccgccgcactgcagtccgccacacctgtggaaagagtgaaactgcaagaagcactgtctcagctggacttccagtgggagaaagtgaacaagatgtacaaggacaggcagggcagattcgaccgcagcgtggaaaagtggcggcggttccactacgacatcaagatcttcaatcagtggctgactgaggcagagcagttcctgagaaagactcagatccccgagaactgggagcacgccaagtacaagtggtatcttaaagagcttcaggacggcatcggccagaggcagacagtcgtcagaacactgaatgccaccggcgaggaaattatccagcagtctagcaagaccgacgccagcatcctgcaagagaagctgggatccctgaacctgcggtggcaagaagtgtgcaagcagctgagcgaccggaagaagcggctcgaagaacagaagaatatcctgtccgagttccagcgggacctgaacgagtttgtgctgtggctcgaggaagccgacaatatcgctagcatccctctggaacccggcaaagagcaacagcttaaagaaaaactggaacaagtgaagctcctggttgaggaactgcccctgagacagggcatcctgaagcaactgaacgagacaggcggccctgtgctggtttctgcccctatctcacccgaggaacaggataagctcgagaacaaactcaagcagacaaacctccagtggatcaaggtgtcccgggcactgcctgagaaacagggcgaaatcgaggcccagatcaaggacctgggacagctcgaaaagaagctcgaagatttggaggaacaactcaaccacctcctgctctggctgagccctatccggaatcagctcgagatctataaccagcctaatcaagagggccccttcgacgtgaaagaaacagagatcgctgtgcaggccaagcagccagatgtggaagagatcctgtccaagggacagcatctgtacaaagaaaagcctgccacacagcccgtgaagcgcaagttggaggacctgtctagcgagtggaaggccgtgaaccggctgctccaagaactgagagccaaacagcccgatctggcccctggcctgacaacaattggagctagccccactcagaccgtgacactggttacacagccagtggtcaccaaagagacagccatctccaaactggaaatgccctctagcctgatgttggaggtgcccgcactggccgacttcaatagagcttggaccgagctgactgattggctgtccctcctggaccaagtgatcaagagccagagagtgatggtcggagatcttgaggacatcaacgaaatgattatcaagcagaaagccaccatgcaggatctcgaacagcgcagaccccagcttgaagaactgatcacagccgcacagaatctgaaaaacaagaccagcaatcaagaagccaggaccatcatcaccgaccggatcgagcggattcagaaccagtgggatgaagtccaagagcatctgcagaaccggcggcagcagcttaacgaaatgctgaaggacagcacacagtggctcgaagctaaagaagaagccgagcaggttctgggacaagccagagccaagctggaatcctggaaagagggaccctacaccgtcgacgccattcagaaaaaaatcaccgagacaaaacagctggccaaggatctgcggcagtggcagaccaatgtggacgtggcaaatgacctggctctgaaactgctccgggactacagcgccgacgacaccagaaaggtgcacatgatcacagagaacatcaacgcctcttggcggagcatccacaagcgggtgtccgaaagagaggctgccctcgaggaaacacacagactgctgcaacagtttcccctggaccttgagaaattcctggcctggctcaccgaggccgaaaccacagctaatgtcctccaggatgccacacggaaagagcggttgctggaagatagcaaaggggtcaaagaacttatgaagcaatggcaggacctccaaggggagatcgaggctcacaccgacgtgtaccacaatctggatgagaacagtcagaagatcctgcggtccctggaaggctccgatgatgctgtgttgctccagcggagactggataacatgaacttcaagtggagcgagctgcggaaaaagtccctgaacatcagaagccatctggaagccagcagcgaccagtggaaaagactccacctgagcctccaagagcttctcgtgtggctgcagctcaaggacgatgagctgagcagacaggccccaatcggcggagattttcccgccgtgcagaaacagaacgacgtgcacagagccttcaagcgggaactcaagaccaaagaacccgtgattatgagcaccctggaaactgtgcggatctttctgaccgagcagcctctggaaggactcgagaagctgtaccaagaacctagagagctgcctcctgaggaacgggcccagaacgttacacggctgctgagaaaacaggccgaggaagtgaataccgagtgggaaaagctgaatctccactccgccgactggcagcggaagatcgatgagactctggaaaggcttcaagaattgcaagaggccaccgacgagctggacctgaagcttagacaggctgaagtcatcaaaggcagctggcagcctgtgggcgacctcctgattgatagcctgcaggaccacctcgaaaaagtgaaggccctgcggggagagatcgcccctctgaaagaaaacgtgtcccacgtgaacgacctggcacggcagcttacaacactggggattcagctgagcccctacaacctgtccacactcgaggacctgaatacccgatggaagctcctccaggtggccgttgaagatagagtgcggcagctgcacgaagcccacagagattttggaccagccagccagcactttctgagcacctcagtgcaaggcccctgggagagagctatcagccctaacaaggtgccctactacatcaaccacgagacacagacaacctgttgggatcaccccaagatgaccgaactgtatcagtccctggccgatctgaacaatgtgcggttcagcgcctaccggaccgcaatgaagctgcggagacttcagaaagccctgtgcctcgacctgctgtctctgagtgcagcatgtgatgccctggaccagcataatctgaagcagaacgaccagcctatggacatcctccagatcatcaactgcctgaccactatctacgaccggctcgaacaagaacacaacaacctcgtgaacgtgcccctgtgcgtggacatgtgcctcaattggctgctgaatgtgtacgacaccggccggaccggcagaatcagagtgctgtctttcaaaaccggcatcatctccctgtgcaaagcccatctcgaggataagtaccgctacctgtttaagcaggtcgccagcagcaccggcttttgcgatcaaagaaggctgggactcctgctgcacgacagcattcagatcccaagacagctgggcgaagtggctagcttcggcggctctaatatcgagcctagcgtgcggagctgcttccagttcgccaacaacaagcccgagattgaggcagccctcttcctggattggatgcggctggaaccccagagcatggtttggctgccagtgctgcatagagtggccgctgccgaaacagctaagcaccaggccaagtgcaacatctgcaaagagtgccccatcatcggcttccggtacagaagcctgaagcacttcaactacgatatctgccagtcttgtttcttctctggccgcgtggccaagggccacaagatgcattaccccatggtggaatactgcacccctaccacctccggcgaagatgtccgggattttgccaaggtgctcaaaaacaagttccgcaccaagcgctacttcgcaaagcaccccagaatgggctacctgcctgtgcagacagttctggaaggcgataatatggaaacccctgtgacgctgatcaatttctggcccgtggatagcgcccctgccagttctcctcagctgtctcacgatgacacccactccagaatcgagcactacgcctccagactggccgagatggaaaacagcaacggcagctatctgaacgatagcatcagccccaacgagagcatcgacgacgaacatctgctcatccagcactactgtcagagcctgaaccaggactccccactgagccagcctagaagcccagcacagattctgatttccctggaatccgaagaacggggcgagctggaaagaattctggccgacctcgaggaagaaaaccggaacctgcaggcagagtacgacagactgaaacaacagcacgagcacaagggactgagccctttgccatctcctccagaaatgatgcccacctctccacagagccccagagatgcagagctgattgccgaggccaaactcctgcgccagcacaaaggcagactcgaggccagaatgcagattctcgaagatcacaacaaacagctcgagtctcagctccaccgcctgcggcaattgctggaacaaccacaggcagaggctaaagtgaacggcaccacagtgtctagccccagcacatccctgcagagatccgatagctcccagccaatgctcctgagagtcgtgggcagccagaccagcgattctatgggcgaagaagatctgcttagccctcctcaggatacaagcaccggcctcgaagaagttatggaacaacttaacaatagcttccccagcagcagaggccgcaacacacctggcaagcccatgcgggaagataccatg

SEQ ID NO: 4 - protein, 3685 aa, Homo sapiens; identical to SEQ ID NO: 2

SEQ ID NO: 5 - DNA, 11055 bp, Homo sapiens
  source 1..11055: mol_type = other DNA
atgctgtggtgggaggaagttgaggattgctatgagcgggaagatgtgcagaagaagacgtttacgaaatgggtcaatgctcaattctcgaaatttggcaaacaacacatagagaaccttttctccgatctgcaagatggtaggagattattggatctcctcgaggggttaactgggcagaaactgcccaaagagaaaggttctaccagagttcacgcgctgaataatgtaaataaagccctgagagttcttcagaacaacaatgtggaccttgtcaatattgggtctacagatatagttgacgggaatcacaaactcactcttgggctcatatggaacatcatactccactggcaagttaagaatgtaatgaagaatattatggcgggcctgcaacaaacgaattccgagaaaatcttgctgtcttgggtaagacagtctactcggaattatccccaggtgaacgtgataaattttaccacttcctggtctgatgggttagcgttaaatgcccttattcactctcaccggcccgaccttttcgattggaattccgtagtttgtcaacaatccgcgactcaacggcttgaacacgcctttaatatcgccaggtatcagctcgggatagagaaacttttagatcccgaggatgttgatactacctatcctgataagaagtcaatactcatgtatataaccagtttatttcaagttctcccacagcaagtttcaattgaggccatacaagaggttgagatgctgcccagaccacctaaggttacaaaagaagagcactttcaattacatcaccagatgcactatagccagcaaattaccgtatcgttagcccaggggtatgagagaacctcttctcccaaaccccggtttaagtcgtatgcctatacacaagccgcctatgttactacctcagaccccacaagatctccgtttcccagccaacacttagaggctcccgaggataaatcctttgggtcttctctgatggagtctgaggtgaatttagaccggtatcaaaccgccttggaggaagtactctcttggcttctgtctgccgaggatactctgcaagcccaaggggagatatctaatgatgtcgaggtggttaaagaccagtttcacacccacgaggggtatatgatggatctcactgcccaccaaggccgtgttgggaatatactacaacttgggtctaaactaattgggaccgggaaactttctgaggatgaggagaccgaggtacaagagcagatgaatcttctgaattcgaggtgggagtgtttacgagttgccagcatggagaaacagtccaatctacatcgcgttctgatggacttacaaaatcagaagctgaaagaattgaacgactggcttaccaaaactgaggagcggactcgtaaaatggaagaggagcctctcgggccagacctagaggatctgaaacggcaggtacaacaacataaagtgcttcaggaagatcttgagcaggaacaagttagggtaaactcgctaacccacatggttgtcgttgttgatgagtcctctggggatcacgccacagccgccttggaagaacaactcaaggtattaggggatcggtgggccaatatttgccggtggactgaggaccggtgggtcctcctacaggatattctgctaaaatggcaacggcttaccgaagagcaatgtctctttagtgcctggttatccgagaaagaggatgccgtaaacaagatccacactactgggttcaaagaccaaaatgagatgctcagctctctccagaaactggccgtccttaaagccgacttggagaagaagaaacagtctatggggaaactctattcattgaaacaggatctgcttagcaccctcaagaataagtctgtcacacagaaaaccgaggcctggttagacaattttgccaggtgctgggataatcttgtccaaaagctcgagaaatctactgcccagatttctcaagctgttactacaactcaaccctctctgactcaaaccactgttatggagactgtgacaactgtaactactagagagcagatactggttaaacacgcgcaagaagagttacctcctcctccgccccaaaagaaaaggcagatcactgtggattcggaaatacgcaaaagactcgatgttgatataactgagctgcattcttggattacacggtctgaggccgtactccaatctccagaatttgcgatctttaggaaagagggcaatttctcagaccttaaggagaaggtaaatgctatagagcgggagaaagctgagaagtttcgcaaactacaagatgcctctcggtctgcccaagccctggttgagcaaatggttaatgaaggggtaaatgccgattccattaaacaggcctctgaacaactgaactcccggtggattgaattctgtcaactgcttagtgagagacttaattggctcgagtatcaaaacaatataatagccttctacaatcagttacagcaacttgagcagatgactactactgccgaaaactggctgaaaatacaacccactactccctctgagcccactgccattaaatcgcagctcaaaatctgcaaagacgaggttaatagactctccgatcttcagccccaaatagaacggcttaaaattcagagtatcgccttgaaagaaaaggggcaagggcccatgtttctcgatgccgatttcgtcgcgtttaccaaccatttcaaacaggtattttccgacgttcaagcccgggagaaagagttacagaccattttcgacactttaccgcccatgcggtatcaagagaccatgtctgccattagaacctgggtccaacaatcggaaactaagctttccatccctcaattatctgtgactgactatgagataatggagcaacggcttggggagctgcaggcccttcaaagctctttacaagagcaacaatctgggctctattatctctccactacggtgaaggaaatgtccaagaaagcgccctccgagatttctcggaaataccaaagtgagttcgaggagatagaggggcggtggaagaaactgagttctcagcttgtagagcattgtcaaaagctggaagagcaaatgaacaagctcaggaaaatccagaaccatatccaaactctcaagaaatggatggccgaggttgacgtcttcttgaaagaggaatggccggctcttggggatagcgagattctaaagaagcagttgaaacagtgccggctcttagttagtgatatccagaccatacaaccctccctgaattctgttaatgagggcggtcaaaagattaagaacgaggccgagcccgagtttgcttcaagactcgagactgaattgaaagagctgaatacacagtgggaccacatgtgtcagcaagtttatgcccggaaagaagcgctcaaaggtgggcttgagaagactgttagcttacagaaagaccttagtgagatgcacgagtggatgacccaagctgaggaggagtatcttgagcgtgactttgagtacaaaacacccgacgagttacagaaagctgttgaagaaatgaagcgggcgaaagaagaggcacaacagaaagaggccaaagtaaagcttctgacagagtctgttaattcggtgatagcccaagcgcctcccgttgcgcaagaggccttgaagaaagaactcgagaccctaaccacaaattatcagtggctctgcactagattgaatgggaaatgcaaaacgcttgaggaggtttgggcttgctggcacgagttgctctcctatttagaaaaggccaacaaatggcttaatgaagtggaatttaagctcaaaaccacagagaatatacccggtggagctgaggagatttctgaagttcttgactctctcgagaatctcatgcggcatagtgaagacaatcccaaccaaatacggattctagcccaaactctgactgacggaggggtcatggatgagctaattaacgaggagctcgagacttttaattccaggtggcgggagttgcacgaggaggccgttcggagacagaaattgttagagcagtccattcaatccgcccaagagaccgagaaatcgctgcatctcatacaggaaagcttgacctttattgataagcagctcgccgcctatatagccgataaagttgacgctgcccaaatgccccaagaagcccagaaaatacagtctgacctgactagccatgagatttctttagaggagatgaagaaacacaaccagggtaaagaggcggcacagcgggttctttcacaaatagacgttgcccagaagaagctccaagacgtctctatgaaatttcggctctttcagaaacctgccaattttgagcaacggttacaagagtccaaaatgattctcgatgaggtcaaaatgcaccttccggccctggaaactaaaagtgttgagcaagaggttgtacagtcccaattaaatcactgcgtcaacctatataaatccctatcggaggttaaatctgaggtcgagatggtaattaagactgggcggcaaatagtccagaagaaacaaactgagaatcccaaagaacttgacgagagagttactgccttgaaactgcactacaatgagcttggggcgaaagttacagagaggaaacagcaactggaaaagtgcctgaaactctctcggaaaatgcggaaagaaatgaatgtgctgaccgagtggctagccgccactgacatggagttaactaaacggtctgccgtagaggggatgccctctaatcttgattcagaggttgcctggggaaaagccacgcaaaaggagatcgagaagcagaaggtccatcttaagtccattacagaggttggggaggccctgaaaaccgttttaggtaagaaagaaaccttagttgaggataaactctcactgcttaactcaaattggatagccgtaactagcagggccgaagagtggctaaatttgcttctggaataccaaaagcatatggagacatttgatcagaatgtcgaccacatcacgaaatggatcattcaggctgacactttactcgatgaatccgagaagaagaagccacagcaaaaggaggacgtactgaaacgactcaaagccgaactcaatgatattaggcccaaagttgattctactcgcgatcaagccgccaatcttatggccaatcggggcgatcactgcaggaaattggtagagccccagattagtgagctcaatcacagatttgccgccatatcccacagaataaagacaggcaaagcctctattccccttaaggagctcgaacaatttaactcggacattcagaaactcttggagcccttagaggccgaaattcaacaaggggttaatctcaaagaagaggacttcaataaggacatgaatgaggacaatgaggggactgttaaggagcttctgcaaaggggcgacaatcttcaacaacggattactgatgaacggaaacgggaggagattaagattaagcagcagcttctccaaacaaaacacaatgcgctcaaagacctcagatctcaacggcggaagaaagctttggagatttctcaccagtggtaccaatacaaaagacaggccgacgatttactaaaatgcctcgacgatatcgagaagaagcttgcttcattacccgaacctcgggacgagcggaaaattaaagagatagatcgcgagctacagaagaagaaagaggagctgaatgccgttagacggcaagccgaagggctatctgaagacggtgcagcgatggcggttgagcccacccaaattcagctgtccaaacggtggagagagatagagtctaagtttgcccaattccgaagactcaattttgcccagattcatactgttagagaggagacaatgatggtgatgactgaggatatgcccttagagatatcgtatgtcccctctacctatctgaccgagattactcatgtgtctcaagccctcctcgaagttgagcaactcttaaatgcaccggatctttgcgccaaagacttcgaagatcttttcaagcaggaggagtcccttaagaacatcaaagactctctccagcaatctagtgggcgcatagatatcattcactcgaagaaaactgcagccctccaaagcgccacacccgttgaacgagttaaactccaagaggccctgtcacagctcgattttcaatgggagaaagtcaacaagatgtataaagaccggcaagggcggtttgacagatctgttgagaaatggcggcggttccactatgacatcaaaatatttaatcaatggctaactgaagccgaacagttcttacggaaaactcagattcccgagaattgggagcacgccaaatacaagtggtacttgaaagagctacaagacgggattggccagcggcagactgttgttcggactcttaatgccaccggtgaggagattatacagcaaagctctaaaactgacgcatctatccttcaagagaaactcggctctcttaatcttcggtggcaagaggtgtgcaaacaattgtcggataggaagaaacggctcgaagagcagaagaatatactgtccgagtttcagcgggatctcaatgagtttgttctctggttagaagaggcggacaacattgcttcaatacccctggagcccgggaaagaacagcagttaaaggagaaactcgaacaagtgaaacttctcgttgaagagctcccactcagacaagggattctgaagcaactgaatgagactggcgggcccgttctggtttctgctcccatctctcccgaggagcaagataagctggaaaacaagctgaaacagactaatcttcagtggattaaagtcagcagggcccttcccgagaaacaaggagaaatcgaagcgcaaataaaagatctcgggcagttagagaagaaattggaggacttagaggagcagttgaatcacctgttactgtggctctccccgattagaaaccaactggagatctacaatcagccgaatcaagaggggccctttgacgttaaagagactgagattgccgttcaagcgaaacagcccgacgtagaggagattttgtcaaaggggcaacacctgtataaagagaaacccgctactcaacccgttaaacggaaactagaggacctatctagtgaatggaaagccgttaatcgcctcctgcaagaactccgggccaaacaaccggatttggcgcccggattaaccactataggtgcctctccgactcaaactgttaccctcgttactcaaccggttgttaccaaagaaactgccatttctaaactggagatgccctcttctcttatgttagaggttcccgcactggccgattttaatcgggcctggaccgagttaactgattggctttctctgttagaccaagtcatcaaatcccaacgggttatggtaggggatttggaagacattaacgaaatgatcattaaacagaaagccactatgcaggatctcgagcaacggagaccccagcttgaggagcttataaccgccgcccagaatctgaagaataaaacgtcgaatcaggaggcccgaactatcatcactgatagaatagagcggattcagaatcaatgggatgaagttcaggagcacttacagaataggcggcaacaactcaacgaaatgctaaaggatagcacccaatggctcgaagctaaggaagaggccgagcaagttttggggcaagctcgcgccaaattagagtcttggaaggaggggccttacaccgttgatgcgatccagaagaagattacagagactaaacagctagcgaaagatctgcggcagtggcaaaccaatgttgatgttgccaatgacttagcgcttaaactcctccgggactactccgccgatgatacacggaaagtgcacatgataaccgaaaacatcaatgcctcttggcggtctatacacaaacgggtttcagaacgagaggcagcccttgaagagactcacagacttctccaacaatttcccttggatctggagaaattcttagcatggcttactgaggccgagaccactgccaatgttttacaagacgccacacggaaagaacggctcctagaggactctaaaggggtgaaagaactcatgaaacagtggcaggacttacagggcgaaatagaggcccacactgatgtttatcacaacctcgacgaaaactctcagaaaatccttaggtctcttgaggggtctgatgatgccgtgcttctgcagagaaggctggacaacatgaacttcaagtggtctgagcttaggaagaaatcgctcaacatacggtcccacctagaggcctcttctgatcaatggaaacggttgcatttatctctgcaggagttattagtatggctccaattaaaagacgacgagttgtctcggcaggcgcccattggtggggattttcccgccgttcagaaacagaacgatgttcaccgggccttcaaacgagagcttaaaacaaaagagcccgttattatgtctaccctcgaaactgttcggatattcctcacagaacaacccctcgaagggttagagaaactgtaccaggagcctcgggagcttccaccggaggaaagggctcaaaacgttactcggttattaaggaaacaggccgaggaggtcaataccgagtgggagaaactgaatttgcactccgccgactggcaacggaaaattgatgagactctcgagagacttcaagagcttcaagaggctacagacgagctagatttgaaattgcgccaagccgaagttattaaggggtcttggcaacccgttggtgatcttctcattgattctctgcaagatcacctagagaaagttaaagccctccggggtgagattgcgcccctgaaagagaatgtatcacacgtgaatgatcttgcccggcaacttactactttaggcattcaactttcaccgtataatttgtccaccctcgaggatctaaatactcggtggaaactcttacaagtagccgttgaggatagagttcggcaattacatgaggctcacagagattttgggcctgcctctcagcacttcttatctacctctgttcaagggccctgggaaagggcaataagccccaataaagtgccctactacattaatcacgagactcaaactacttgctgggaccaccccaaaatgacggaattataccagtctcttgccgatttaaacaatgtccggttttctgcctatcggacagccatgaaactgcggagacttcagaaagccttatgcctcgatcttctttcactttctgctgcctgtgacgccctggaccaacacaacctaaaacaaaatgatcaacccatggacattttgcagattataaactgcctcacaactatttacgaccggttagaacaggagcataacaacttggtaaacgtacccctttgcgttgatatgtgcctaaactggctcctgaacgtatacgatacaggacggactgggaggattcgggttctaagctttaagaccgggattatttctctgtgcaaggcccatctggaggacaaatacaggtatctcttcaaacaagtggcctctagtactgggttttgcgatcaaagaaggttggggttactgttacacgactctattcaaattccccggcaattaggggaagttgcctcatttggtgggtctaatattgagccctctgtaagatcttgcttccaattcgccaacaataaacccgaaatcgaggccgccttatttctcgattggatgcggttagaaccccaatctatggtttggttacccgtgcttcacagagttgcggcagctgagacagccaaacatcaagcgaaatgcaacatatgcaaagagtgtcccataatagggtttcggtatcggtcgttgaagcattttaactacgacatatgccagtcctgcttcttttcagggagagttgccaaagggcacaaaatgcattatcccatggttgagtattgcacacccactacttctggggaggatgttcgggattttgctaaagttctgaagaataagttccggactaaacggtattttgccaagcatcccagaatggggtatttacccgttcagacagttctggagggcgataatatggagaccccggtgactctgatcaatttctggcccgttgatagtgcacctgcctctagcccgcagctgtctcacgacgacacccactctcggatagagcactatgcctccaggttggccgagatggagaatagtaatgggtcttacctgaacgattctatttcacccaatgagtcaatagacgacgagcatctactcattcagcattactgccagtctctcaatcaagattccccactgtctcagcccagatctcccgcccagattttgatttcactcgagtctgaagagagaggggagctcgaaaggattttagccgacctcgaggaggagaatcggaatttacaagccgagtacgatcgcctaaaacaacaacacgagcacaaagggctgtctcccttaccgtctccgcccgagatgatgcccacttctccccaatcacccagagatgccgagcttatagccgaggccaaactccttcggcaacataaagggcggctcgaagcgagaatgcaaattcttgaggatcacaataagcaactcgagtctcagcttcacagattaagacagctccttgaacaaccccaggccgaggctaaagttaatgggactactgtttcgtctccgtctacttcattacagagatctgattcttcccaacccatgttactgagagttgttggcagccaaacttctgattctatgggtgaagaagacttgttatctccgccgcaggatacctccactgggctagaagaggttatggagcagcttaacaattcctttccctcttctaggggcagaaatacacccggtaagccgatgcgggaggacaccatg

SEQ ID NO: 6 - protein, 3685 aa, Homo sapiens; identical to SEQ ID NO: 2

SEQ ID NO: 7 - DNA, 11055 bp, Homo sapiens
  source 1..11055: mol_type = other DNA
atgctctggtgggaagaggtcgaggactgctatgagcgggaggacgtccagaagaagacctttacgaaatgggtgaatgcccaattctctaaattcggcaagcaacatatcgagaatctgtttagcgacttacaagatgggcggcggttactggacttactggaggggctgactgggcagaaactgccgaaggagaaagggtccactagagttcacgcacttaacaacgttaacaaagccctgcgggttctccagaacaacaatgtcgacttagttaatatagggtcaacagacattgtagatgggaatcacaaacttactctggggctcatttggaatataattcttcactggcaagttaagaacgtcatgaagaacattatggccgggctccaacagactaactcggagaaaatactgctctcttgggttagacagtctacccggaattatccccaggtgaatgtgattaattttaccacgtcctggtctgatgggcttgcccttaatgccctgattcactctcatagacccgacctctttgactggaacagtgtagtttgtcaacaatctgccacccagagacttgaacacgcgtttaacattgcgagataccaactagggatagagaaattgcttgaccccgaggatgttgacactacttatcccgacaagaaatccatactgatgtacattaccagtctattccaagttcttccccaacaagtttctattgaggccattcaggaggtggagatgctaccccggcctcccaaagttaccaaagaggagcattttcaactgcaccaccagatgcactattctcagcagataacggtgtccctggcccaagggtatgagagaacttcttctcccaaaccccggttcaaatcctatgcctatacacaagccgcgtatgttacaacctctgatcccactcggtctccctttccctctcaacacctcgaagcgcccgaggataaatcctttgggtcttctttaatggagtctgaggtcaacctcgatcggtatcaaactgcgcttgaagaggtgttatcctggcttttgtctgccgaggatactcttcaagcccaaggggagatttctaatgatgtcgaggtggtcaaagatcagtttcacacccacgaggggtacatgatggatttgactgcccaccaaggacgggttgggaatatactccagctcgggtctaaactcatcgggacagggaaactcagcgaggacgaggaaacagaggtccaggaacagatgaatctcctgaattcacgttgggagtgtcttagagttgcctctatggagaaacaatctaacctacatcgggttttaatggatctccagaatcagaagctcaaggagctaaatgattggctaaccaaaaccgaggagagaacacggaaaatggaggaggaaccgttagggcccgacctagaggacttaaaacggcaagttcaacaacacaaagttctccaagaagatttagagcaggagcaagttagagtcaattcactgactcacatggttgtagtggtcgatgagtcttctggggaccacgccactgcagcccttgaggagcagttaaaagttcttggggatagatgggccaatatttgccggtggacagaagatcggtgggtacttctgcaggacattctactgaaatggcaacggttaaccgaggaacaatgcttattctctgcctggttatctgagaaagaagacgccgtcaacaagatacacacgactggattcaaagaccaaaacgaaatgctgtcatctctgcagaaattggccgtgttaaaagccgatctcgagaagaagaagcagtctatggggaagctttattcgctgaaacaagaccttctttctacccttaagaataaaagtgtgacccaaaagaccgaagcctggttggataattttgcccggtgctgggataatctcgtacaaaagctcgagaaatctacagcccaaatttcccaagccgttactactactcaaccctctcttactcagactactgttatggagactgttactactgttactacacgggagcaaatactcgtaaaacacgcccaggaagaattaccaccgccaccgccccagaagaaacggcaaattactgtggactctgagattcggaaaagattagacgtcgatattaccgagcttcattcgtggataactagatctgaggccgttcttcagtctcccgagtttgctatatttcggaaagaggggaactttagtgatttaaaggagaaagtaaatgccatagagagagagaaagccgagaaatttcggaaactccaggatgcctcacggtccgcccaagccttggtggaacaaatggtcaatgagggcgttaatgccgattctatcaaacaagcctccgaacaactcaattctcggtggattgagttttgccagttactctccgaacggctcaattggttagagtaccaaaacaatataattgcattctataaccagctccagcaactcgagcaaatgactacaactgcggagaactggcttaaaatacagccgacaactccgtctgagcccacggccataaagtcgcagctaaagatatgcaaggacgaggtcaatcggttatctgatcttcaaccccagattgagcggctcaaaatacagtctatagccctgaaagagaagggacaagggcccatgtttcttgatgccgacttcgtagcttttacaaaccatttcaagcaagtcttttctgacgttcaagcgagagaaaaggagctgcagaccattttcgacactcttccacccatgcggtatcaagagaccatgtctgccattcggacctgggtacaacaatctgagactaaactctctatcccgcagctatccgtgaccgactatgaaataatggagcaacgtctcggggagctccaagcccttcaaagctctctacaagagcaacagtccgggttatattatctcagcaccaccgtaaaagagatgtctaagaaagctccctccgagatttcgagaaaataccagtctgagtttgaggagattgaagggagatggaagaaattatcgtctcaactcgtggagcactgccagaaactggaggagcagatgaataagttacggaaaattcagaaccatatccaaaccctgaagaaatggatggccgaggttgacgtgttcttaaaagaagagtggcctgccctaggagactcagagatccttaagaagcaacttaagcaatgccggctgttagtttccgacattcaaactatccagccctccctaaacagcgttaatgaaggcgggcagaaaatcaagaacgaggctgagcccgaatttgcctctcggctcgagactgagctcaaagagcttaatacacagtgggaccatatgtgccaacaagtatatgcccgcaaagaggcacttaaaggtgggcttgagaaaaccgtttctctgcaaaaggatctttcggaaatgcacgagtggatgactcaagcagaggaggagtatctcgagcgggacttcgagtataaaactcccgacgaattacagaaagcggttgaagagatgaaacgggcaaaagaggaggctcaacagaaagaggccaaagtgaaactgttaaccgagtcggttaattctgtgattgcccaggctccacccgttgcccaggaagcccttaagaaagaactcgagacactcactacgaactatcaatggctctgcactcggcttaatggcaagtgtaaaacgttggaggaggtttgggcctgctggcacgaactactctcttatctcgaaaaggcgaacaaatggctgaatgaggtggaattcaaactcaaaaccactgaaaatattcccggtggtgccgaggaaatttctgaagtgttggactcacttgagaatctgatgaggcattctgaggacaatcccaatcagatacggattcttgcccaaactctcactgatggcggggttatggatgagctgattaatgaagagctagagaccttcaattcaagatggcgggagctccacgaagaggccgttaggcggcaaaagctcctcgaacagtctattcaatctgcccaagagactgagaaaagcttacaccttatccaagagtctctcactttcatcgacaagcaattggccgcctacatagcggataaagtagacgccgcccaaatgccccaagaagcgcagaagatccaatccgatttgacttcccacgagatttccctagaggagatgaagaaacacaatcaggggaaagaggcagcccaacgggttctttcccaaattgatgttgcccagaagaagctccaagatgtaagcatgaagttccgtctatttcaaaagccagccaattttgagcaacggttacaagagtctaagatgatccttgatgaggtgaaaatgcaccttcccgcgctcgagaccaaatctgttgaacaagaggttgtccaatcgcaattgaatcactgtgtgaacctgtataagagtcttagcgaggttaaatctgaggtcgagatggttattaaaaccgggcggcaaattgttcaaaagaaacagacagagaatcccaaagagcttgacgagcgggttactgccttgaagctacactacaatgaactaggggccaaagtaaccgagagaaaacagcaactggagaaatgtctcaaactgtcccggaaaatgcggaaggagatgaatgttttgaccgagtggcttgccgccactgacatggagcttactaaacggtctgccgtagaggggatgccctctaatcttgattccgaggttgcctggggtaaagccacccagaaggaaatcgaaaagcagaaagtacatcttaaaagtataaccgaagttggggaggccctgaaaactgttcttgggaagaaagagaccctcgtggaggacaaactgagtctgcttaacagtaactggatagccgttacttctcgtgccgaggagtggcttaaccttttactcgagtatcagaaacacatggagacatttgaccagaatgttgaccacatcaccaagtggataattcaggccgacactttattagacgagtctgaaaagaagaaaccccaacaaaaggaagacgtgttaaaacggctgaaagccgagctcaatgacatacggcccaaagtagattctactcgggatcaggccgccaatctgatggccaatagaggggatcactgcagaaaactcgttgagccccaaatatctgagctcaatcacagatttgccgccatttcacaccggatcaaaactgggaaagcctcgatacccttaaaagagctggaacagtttaactctgacattcagaaacttcttgagccccttgaggccgagattcaacaaggggttaatcttaaagaggaggacttcaacaaagacatgaatgaggataatgaggggaccgtgaaagaactactccagaggggtgacaatctccaacaaaggattactgatgagcggaaaagagaggaaatcaaaattaagcaacagttattgcaaaccaagcataatgctctcaaagaccttcggtctcaacgccggaagaaagcgttggaaatatctcaccagtggtaccaatataaacgccaagccgacgatctgttaaaatgcctggacgatatagagaagaaactcgcgtctctccccgaacccagagatgagcgcaagatcaaagaaatcgaccgggagcttcagaagaagaaagaggagctgaatgccgttcgcagacaagccgagggcctttctgaggatggggctgccatggccgttgaacccactcaaatacaactttctaaacggtggagagagatcgagtcaaaattcgcccaatttcggcggctcaatttcgcccaaatacacacagttcgggaggagactatgatggttatgactgaggatatgcccctggagatttcttatgttccctctacctacctaactgaaattacccacgtttctcaggccttgcttgaggttgagcagttactcaatgcgcccgatctctgtgccaaagactttgaggatctcttcaaacaagaggagagtctaaagaatattaaagacagcctccagcaatcttctgggcggatcgacattattcacagcaagaaaactgctgctctgcaatctgctactcccgttgagcgggttaaactccaagaggctctctcgcaattagatttccaatgggaaaaggtaaacaaaatgtataaagatcggcaagggagatttgatcggtctgttgagaaatggcggagatttcattatgacattaaaatcttcaatcagtggttaacggaggccgagcaatttcttcggaaaactcaaattccggagaattgggaacatgccaagtacaagtggtatcttaaagaacttcaggatgggatagggcaaagacaaactgtggttcggacattaaatgccactggcgaggaaattattcagcagtcatccaaaaccgatgcctccatattacaggagaaactagggtctcttaatctccggtggcaagaggtgtgcaaacaactgtcagatagaaagaaacggttagaggagcagaagaatatactctcggagtttcaaagggatctaaacgaattcgtcctttggttagaggaagctgacaatattgctagcattcccctcgagccggggaaagaacagcagctgaaggagaaactggaacaggtgaaattacttgttgaggagctgcccctcagacaagggattctcaaacaactgaatgagactggtgggcccgttcttgtttctgcccctatttctccggaggagcaggataaactcgagaataaactgaaacagaccaacctccaatggataaaagtatcccgggccttacccgagaaacagggtgaaattgaggcgcaaataaaagacctgggccaacttgagaagaaattggaggatctcgaagagcaactgaaccacctcttactatggctgtctcccattaggaaccagctggaaatctataatcagccgaatcaagaggggccgtttgatgttaaagaaaccgagatagccgtacaagccaagcaacccgatgtcgaggagatactctctaaagggcaacatctctacaaggaaaagcctgccactcagcccgtcaaaaggaaactagaggatctgtcttctgaatggaaagccgtaaatcggctccttcaggaattacgggctaaacaacccgatcttgccccgggattgactactattggggcctcgcccactcaaaccgttactttagtgactcaacccgttgtaactaaagagactgccatttccaaacttgagatgccctcttctttgatgttagaggttcccgcactcgccgattttaatagagcctggaccgagcttactgactggctctctttactcgatcaagtcatcaaatcccagagagtgatggttggggatctggaggatattaacgagatgataattaagcagaaagcgacaatgcaggatttagaacaacggagaccgcaattagaggagctcattactgcggcccaaaacttaaagaacaagaccagcaatcaagaggccagaacgataatcactgaccgaattgagagaatccagaatcaatgggatgaggtgcaggaacacctccagaatcggagacaacagttgaacgagatgctcaaagattccactcagtggcttgaagcgaaagaggaagcggaacaggtccttggccaagccagagccaaactggagtcctggaaagagggcccctatactgtggatgcgatccagaagaaaataaccgaaacaaaacagttagcgaaagatctgagacaatggcaaactaacgtagatgttgccaatgaccttgccctgaaactactccgggattattccgccgacgatacaagaaaggttcacatgatcacagagaacataaatgcctcttggcggtctatacacaaacgggtatctgaaagggaggccgctctcgaagaaacccacagactgcttcagcagtttcccctagacctggagaaatttcttgcctggcttaccgaagccgaaacaacagccaatgttttacaggatgccacacggaaagagcggctactcgaggactctaaaggggtcaaggaactgatgaaacaatggcaggatcttcaaggtgaaatagaggcacacactgacgtttatcataacctcgacgagaattcacaaaagatcctacggtcccttgaggggtctgatgacgccgttctccttcaaaggcggttggataacatgaattttaaatggtcggagttacgcaagaaaagtctcaacattcggtctcatctggaggcctcctctgaccagtggaaacggttacacttatctctccaggaactcctcgtttggttacaactgaaagacgatgagttgtctcggcaagctcccataggcggggattttcccgccgttcagaaacagaacgacgttcacagggcctttaagcgggagcttaaaactaaagagccggtgattatgtccactctcgagactgttaggatatttctcaccgagcaaccgcttgagggccttgagaaactctatcaagagcccagagaattacctcccgaagagcgtgcccagaatgttaccagactcctaagaaaacaagcggaggaggttaatactgagtgggagaaactgaatctccactctgccgactggcaacgcaagattgatgagacattggagcggctccaagaattacaagaggccaccgatgagcttgaccttaaattacggcaagcggaggtaataaaaggtagctggcaaccggtgggagacctgcttatcgattctttacaggatcatctcgagaaagttaaagccctccggggcgagattgctccgctcaaagagaatgtgtctcacgtgaatgacttagcccggcaactcactactttggggattcaactgtctccctacaatcttagtactctggaggacttgaatactcggtggaaacttctgcaagttgctgttgaggatagagttagacagcttcacgaggcccacagagactttgggcccgcttcccaacactttctctctacttctgtacaaggtccctgggaacgagccatttctccgaacaaagtcccttactatataaatcatgagactcaaaccacttgctgggatcaccccaaaatgacggagctatatcaatctctggccgatctgaacaatgttcggttttctgcctacagaaccgccatgaaactgagacggctgcagaaagccttatgcctcgacctcctatctctttctgccgcctgtgatgccttagaccaacacaaccttaagcaaaatgaccaacccatggacattctccagattatcaactgcctcactacgatttatgacagactggaacaagagcacaacaacctcgttaatgttcccctctgcgtcgatatgtgcttgaattggctcctcaatgtatatgacaccgggagaactgggagaattcgcgttctgtcgtttaagactggtatcatatctctttgcaaagcccacttagaggacaaatatcggtatctatttaaacaggttgcctcttcaactgggttttgcgatcaacggcggcttggtctcctcctgcacgactctattcaaataccacggcagctaggggaggtggcctcatttggcggttctaatattgaaccctctgtccggtcttgttttcagttcgcgaacaataagcctgagatagaggctgccctattcttagattggatgcggttagagccccaatctatggtgtggctccccgtacttcatagagttgcggcggcggagactgccaaacaccaagctaaatgcaacatatgcaaagagtgtcccattattgggtttcggtatcggtctcttaaacacttcaactatgacatatgccaatcctgcttcttttctgggcgggttgccaaagggcataaaatgcactatcccatggtagagtattgtactcccactacttctggggaggacgttagagactttgcgaaggtactcaagaacaaatttcgaacgaaacgctattttgcgaaacaccctcggatggggtatcttcccgttcaaactgtgctagagggcgacaatatggagactcccgtaacgcttatcaacttctggcccgttgattctgcgcccgcctcttctccacagctctctcatgatgatactcattctcggatagagcactatgcctccagacttgccgagatggagaattccaatggttcttacctgaatgactctatttccccaaacgagtctattgatgatgagcacctccttattcagcactattgccagtctctaaatcaagactctcccctctctcaacccagatctccggcccagattttaatatcactcgagtctgaagaacgcggcgagctggagagaattctagccgatttagaagaggagaatcggaatcttcaagccgagtatgacaggctgaaacaacaacacgagcacaaaggtctttctccccttccctctccacccgagatgatgcccacttcccctcagtccccaagagatgccgagctcattgctgaggccaaactgttaagacagcacaaagggcggttagaagcccggatgcaaattctcgaggatcataataaacagctcgagtctcaacttcaccggttacggcagctgcttgagcagccgcaagccgaagccaaagttaatgggacaactgtttcttcaccctctacctctctccagcggtctgattcttctcagcccatgttattacgggttgttgggtctcagacatctgactctatgggcgaggaggatcttctctctcctccccaagatacttctacaggcttagaggaggttatggagcagctcaacaattcttttccctcttccagagggcggaatacacccgggaagccgatgagagaggataccatg

SEQ ID NO: 8 - protein, 3685 aa, Homo sapiens; identical to SEQ ID NO: 2

SEQ ID NO: 9 - DNA, 11058 bp, Homo sapiens
  source 1..11058: mol_type = other DNA
atgctctggtgggaagaggttgaggactgctacgagcgggaagacgtccaaaagaagacattcaccaaatgggtcaatgcccaattctccaagttcggtaagcagcacatcgagaatttgttctctgatctccaggacggaagacggctcctggacctgttggagggactgaccggccagaaattgcccaaagaaaagggctctacccgggtgcacgcactgaacaatgtcaacaaggccctgagggtgctccagaacaacaacgtggacctcgtgaatatcgggagtaccgacatcgtcgacgggaatcacaagctgaccctcggactgatctggaacatcattctgcattggcaggtgaagaacgtcatgaagaacattatggccggtttgcagcagacaaactccgagaagatcttgctgtcctgggttcggcagtcaacccggaactatccccaggtgaacgtcattaactttactacctcttggtccgacggccttgccttgaacgccctgatccactcccacaggccagacctcttcgactggaacagcgtggtctgccagcaatctgccacccagcggcttgaacacgccttcaatatcgcacgctaccagttgggcatcgaaaagcttctcgaccccgaagacgtggacaccacctaccccgacaagaagagcatcctgatgtatatcacctccctgttccaggtgctccctcaacaggtgagcatcgaggccatccaggaagtcgagatgctgccacgccctcccaaggtcaccaaggaggagcacttccaactccatcaccagatgcattattctcagcagatcacagtgtcactggcccagggttacgagcggacatcctcacccaagccccggttcaagtcctacgcctacacccaggctgcttatgtcaccacctcagacccaacccggagtccctttccctcccagcatctggaggctcccgaagacaagtcctttggatcctccttgatggagagcgaagtcaacctcgaccggtaccagaccgctttggaagaggtgctctcctggctcctgtccgcagaagacaccctccaagcccagggtgaaatcagcaacgacgtggaggtggtgaaggatcagttccacacacacgagggctacatgatggacctgactgcccatcagggacgcgtgggcaacattcttcagctgggctccaagctgatcggcaccggcaagctgtccgaggacgaagagaccgaagtgcaggaacagatgaatttgctgaactcccgctgggagtgcctcagagtcgcatcaatggagaaacagtccaacctgcacagggtgttgatggatctccagaatcagaagttgaaggaactgaacgactggctcaccaagaccgaagagagaacccggaagatggaagaggagccactgggccctgatcttgaggatctgaagcggcaggtccaacagcacaaggtgctccaagaggacctcgagcaggagcaggtccgcgtcaactccctgacccacatggtggtggtggtggatgaatcaagcggcgaccatgcaaccgcagccttggaggaacagctcaaggtcctgggtgaccgctgggccaacatctgccggtggacagaggaccggtgggtgctgcttcaggacatcctcctgaagtggcaacgcctgaccgaggagcaatgcttgttcagcgcctggctctctgagaaggaggatgcagtcaacaaaatccacaccaccggcttcaaggatcagaacgagatgctgagctccctgcagaagctcgccgtcctgaaggctgacctcgagaagaagaagcagtccatgggtaagctgtactccctgaagcaggacctgctctctactctgaagaataagtccgtcacccagaagaccgaagcctggctcgataacttcgcccggtgctgggacaacctcgttcagaaactcgagaagagcaccgctcaaatctcccaggccgtcacaaccacccagccctctctgacccaaaccacagtcatggagaccgtgaccaccgttaccacccgggagcagatcctcgtcaagcacgcccaagaggaacttcctcctcctcctccacagaagaaaaggcaaatcaccgtcgactccgaaatccgcaagcgcctcgacgtggacatcaccgagctccatagctggattacccgcagcgaggcagttctgcagtcccctgagttcgccatcttccgcaaggagggtaatttctccgaccttaaagagaaggtcaacgccatcgagcgcgagaaggccgagaagttccgcaagcttcaggacgcctcacggtcagcacaggcattggtggagcagatggtcaacgagggcgtcaacgccgatagcatcaagcaggcttccgaacagctgaacagccggtggatcgagttctgccagctcctcagcgaacggctcaactggctcgagtatcagaacaacatcatcgccttctataaccagctgcaacaacttgagcaaatgaccaccaccgccgagaactggctgaagatccagcccaccaccccttccgaacccaccgccatcaagtctcagctgaagatttgcaaggacgaggtcaatcggctgagtgacctccaaccccagattgagcggctcaagatccaaagtattgccctgaaggagaagggccaaggccccatgttcctcgacgccgacttcgtcgccttcaccaaccacttcaagcaagtcttcagtgacgtgcaggcccgcgaaaaggagcttcagacaatcttcgatacccttcctcctatgcggtaccaggagaccatgtcagcaatcaggacctgggtgcagcaatcagagaccaagctgtcaatcccacagctcagcgttaccgactacgagatcatggagcaaaggttgggagagctgcaagccctgcagtcctccctgcaggagcagcagtccgggctctactacctgagcaccaccgttaaagagatgtccaagaaggcccctagcgaaatcagtagaaagtaccagtcagagttcgaagagattgaggggcggtggaagaagctcagctcccaacttgtggagcactgccagaagctcgaagagcaaatgaacaagctccggaagattcagaaccacattcaaaccttgaagaagtggatggcagaggtcgacgttttcctcaaggaggagtggcccgcacttggggactccgagattcttaagaagcaactgaagcaatgccggctgctggtgtctgacatccaaaccatccagccctccctgaactctgtgaacgaaggaggtcagaagatcaagaacgaggctgagcccgagttcgcctcacggctggagaccgaactcaaagagctgaatacccagtgggaccacatgtgccagcaggtgtacgctcggaaggaagctctgaagggaggtctggagaagaccgtctctctgcaaaaggacttgagcgagatgcacgagtggatgacccaagctgaggaggagtacctggagcgggatttcgagtacaagacacccgacgagctgcagaaagccgtcgaagagatgaagcgggcaaaggaggaggcacagcagaaggaggctaaagtcaagttgctgaccgagtccgtgaactccgtgatcgctcaggccccaccagttgcccaagaagcattgaagaaggagttggaaaccctcaccaccaactaccagtggctctgcaccaggctgaacgggaagtgcaagacccttgaggaggtgtgggcttgctggcatgagctgctctcctatctcgagaaggccaacaaatggctgaatgaggtggagttcaagctgaagaccaccgagaatatccctggtggggcagaggagatctccgaggttctcgactcacttgagaatctgatgcggcatagcgaggacaaccccaaccagatcagaatcttggctcagacccttaccgacggaggggtgatggatgagctgatcaacgaagagctcgagaccttcaacagcaggtggcgcgagttgcacgaggaggctgtcaggcggcagaagctcctcgagcaaagcatccagtctgcccaggagacagagaaaagtctgcacctgatccaggaatccctgaccttcatcgataagcagctggctgcttacatcgccgacaaggtggacgctgctcagatgccacaagaggctcagaagatccagtccgacctcacctcccatgagatctccctcgaggagatgaagaagcacaaccagggtaaggaggctgcccagagggtcctgtcacagatcgacgttgcacagaagaagctccaggacgtcagcatgaagttccggctgttccaaaagcccgcaaattttgagcagagactccaggagtccaagatgatcctcgacgaggtgaagatgcacctgcctgccctggagaccaagtctgtggagcaggaggtcgtgcaatcccagttgaaccactgcgtgaacctgtacaagagcctgtccgaggtcaaaagcgaggtcgagatggtgatcaaaaccggcaggcaaatcgtgcaaaagaagcaaaccgagaaccccaaggaactggatgagcgggtgaccgcccttaagctccactacaacgagctcggtgccaaagtcaccgaacggaagcagcagctcgagaaatgcctcaagctgagccgcaagatgcggaaggagatgaacgtcctgactgagtggctggctgccaccgacatggagctgaccaagaggagcgctgtggagggaatgccctccaacctggatagcgaggtcgcctggggtaaggccacccagaaggaaattgagaagcagaaggtccacctgaagtccatcaccgaggttggggaagccctgaagaccgtcctgggcaagaaggagaccctggtggaagacaagctgtcactcctgaactccaactggatcgcagtgaccagtcgcgccgaagagtggctcaacctcctcctggagtaccagaagcacatggagaccttcgaccagaacgtcgaccacatcaccaagtggatcatccaggcagacacccttctcgacgagtctgaaaagaagaagccccagcaaaaggaggacgtcctgaagagactcaaggctgagctcaacgacatccgcccaaaggtggactccactcgggaccaggcagctaacttgatggccaataggggcgatcactgccggaagctggtggagccccagatcagcgagctcaaccacaggttcgccgccatcagtcaccggatcaagactgggaaggcctccatcccacttaaggagctggaacagttcaactccgacattcagaagctgctcgaacctctggaggccgagatccagcaaggggtcaacctgaaggaggaggacttcaacaaggatatgaacgaggataacgagggaaccgttaaggagcttctccagcgcggcgacaacctgcaacagcggatcaccgacgagcggaaacgggaggagatcaagatcaagcaacagctgctgcagaccaagcataacgccctgaaagacctgcggtctcaacggcggaagaaggccctggaaatctcccatcagtggtaccagtacaagcggcaggccgatgacctgctcaagtgcctggacgacattgagaagaagcttgccagtctgcctgagccccgcgatgagagaaagatcaaggagattgaccgggagcttcaaaagaagaaggaggagctcaatgctgtccggaggcaagctgagggtctgtccgaagacggtgccgctatggctgtggagcccacccagatccagctgagtaagcggtggcgcgaaatcgagtcaaagttcgcacagttccggcggctgaacttcgcccagatccataccgtccgggaggaaaccatgatggtcatgaccgaggacatgcctctggagatttcttacgtcccttccacatacctgactgaaatcacccacgtgtcccaagcccttctggaagtggagcagctcctgaatgcccctgacttgtgcgccaaggatttcgaggacctcttcaagcaggaggagagcctgaagaatatcaaggactctctccaacagtcctccggtcggatcgacatcatccactccaagaagactgcagccctccagtcagcaacccctgtcgagcgcgtgaagctccaagaagccctctctcagttggacttccaatgggagaaagtcaacaagatgtacaaggaccggcaagggcgcttcgaccggtctgtcgagaagtggcggaggttccactacgacatcaagatcttcaaccagtggctgaccgaggctgaacagttcctgcggaaaacccagatccctgagaactgggagcacgccaagtacaagtggtacctcaaagagttgcaggatgggatcggccaaaggcagaccgtggtccggaccctcaacgccaccggtgaggagatcatccagcagtcatccaagaccgatgcatccatcctgcaggagaagctcgggtctctgaacctgcggtggcaggaggtctgtaagcagctctccgaccggaagaagcggctggaagagcaaaagaacatcctgagcgagttccagcgcgatctgaacgagttcgtgctctggttggaggaggctgataacatcgccagcattcccctggagcccggtaaggaacaacagcttaaggaaaagctggagcaggttaagctcctggtggaggagctgccactccggcaaggaatcctcaagcagcttaacgagactggtggacccgtgctcgtctccgctcccatcagcccagaagagcaggacaagctcgaaaacaagctgaagcagaccaatctgcaatggatcaaggtgtcccgggctctccctgagaagcagggagaaatcgaggcacagatcaaggacctgggccagcttgagaagaaactcgaagacctcgaggagcaacttaatcacctgctgctgtggctctctcccatccggaatcagctggaaatctacaaccaaccaaaccaggagggccccttcgacgtgaaagaaaccgagatcgccgtgcaggctaagcagcccgacgtcgaggagatcctgtccaaggggcagcacctgtataaggagaagcctgctacacagcccgtgaagcggaagcttgaagacctcagcagcgagtggaaggccgtcaaccggctcttgcaagaacttcgggccaagcaaccagacctggctccaggcctgacaaccatcggcgcttctccaacccaaaccgtcaccctggtgacccagccagtcgtcactaaagaaaccgcaatctctaagctcgagatgccctcctccctgatgctggaagtgcccgctctggctgacttcaaccgcgcatggaccgagcttaccgattggctgagcctgctcgaccaggtcattaagtcccagcgcgtgatggtgggtgatctggaggacattaacgagatgatcattaagcagaaggccaccatgcaggatctggagcaacggcggccacaactggaggaactgatcaccgctgcccagaatctcaagaacaagacctctaaccaggaggctcgcaccatcatcaccgataggattgagcggatccaaaaccagtgggacgaggtccaagagcatctccaaaaccggcggcagcaactgaatgaaatgctcaaggacagcacccaatggctggaggccaaggaagaagcagaacaagtgcttgggcaggcaagggctaagctggaatcctggaaggaggggccatacaccgtggacgcaatccagaagaagatcaccgagaccaagcagcttgccaaggatctgcgccagtggcagaccaacgtggacgtcgcaaacgacctggcactcaagctgctccgggattactccgccgacgatacccggaaggtgcacatgatcaccgaaaacatcaacgcctcctggcgctctatccacaagcgggtgtctgagagggaggctgctctcgaggagacccacagactcctgcagcagttccctctcgaccttgagaagttcctggcctggctcacagaggctgagaccaccgccaacgtcctccaggacgctaccaggaaggagcgcctcttggaggacagcaaaggcgtcaaggaactcatgaagcagtggcaggatctccagggcgagattgaggcccataccgacgtgtaccacaacctcgacgagaactcccagaagatcctgagatccctcgagggtagcgacgatgcagtcctgctgcaacgcaggctggataacatgaacttcaaatggtccgagctgcgcaagaagtccctcaatatccgcagtcatctcgaggctagctccgaccagtggaagcggctgcacctctccctccaagagctccttgtctggctgcagttgaaggatgacgaactgtcacggcaggctcccatcggtggcgattttccagctgtgcagaaacagaacgacgtgcaccgggccttcaagcgcgaactcaagaccaaggagcccgtgatcatgtccaccctcgagaccgtgcggatcttcctgacagagcagcccttggagggcctcgagaagctgtaccaggaacccagagagcttccacccgaagaaagggcacaaaatgtgacccggcttctgcggaagcaggccgaagaggtcaacaccgagtgggagaagctgaacctgcactccgccgattggcagcggaaaatcgacgagacactggagcggctgcaagaactgcaggaagccacagacgagctcgacctcaagctgcgccaggcagaggttatcaagggctcttggcagcccgtcggagacttgctgatcgacagcctccaggaccacctggagaaggtgaaggctctgcgcggagaaatcgcccctctgaaagagaacgtcagtcacgtgaacgacctcgctcggcagctcaccaccctggggatccagctcagcccctacaacctgagtaccctggaggacctgaatacccgctggaaactgctccaggtggcagtggaggatcgggtccggcagcttcacgaggctcatcgggacttcggcccagctagccagcacttcctgtccacctccgttcaagggccctgggagagggcaatctctcctaacaaggtcccctactacatcaaccacgagactcagaccacctgctgggaccatcccaagatgaccgagctctaccagtctctggccgacctgaacaacgtgcgcttcagcgcttaccggactgcaatgaagctgcgccgcctgcagaaggcactctgcctcgacctcctgtcactcagcgcagcttgcgacgcccttgaccagcataacttgaagcaaaacgaccagcccatggatatcctgcagatcatcaactgcctcaccacaatctacgaccggcttgagcaagagcacaataatctggtgaacgttcccctctgcgtggacatgtgcttgaactggctgctgaacgtgtacgacaccggtcgcacaggaagaatccgggtgctgtcattcaagaccggaatcatctccctgtgcaaggcccacctggaggacaagtaccggtacctgttcaagcaggtggctagctccaccgggttctgcgaccagagaaggttggggctgctgctgcacgatagcatccagattcccaggcagctcggcgaagtcgcctccttcggaggctccaacatcgagccatccgtccggagctgctttcagttcgctaacaacaagccagagatcgaggcagccctgttcctggactggatgcggctggaaccacagtccatggtctggctccccgtgctgcacagagtcgccgctgccgaaacagccaagcaccaggccaagtgcaatatctgtaaggagtgccccatcatcggcttccggtaccggtccctcaagcacttcaactacgatatctgccagtcctgcttcttcagtggccgggtggccaaaggtcacaagatgcactaccccatggtggagtactgtacacccactacctccggcgaggatgtgagagacttcgcaaaagtcttgaagaacaagttccggaccaagcggtatttcgccaagcatccacggatgggctacctccccgtccagaccgtgctcgagggcgacaacatggaaaccccagtcaccttgatcaacttctggcccgtggatagcgctccagctagctcccctcagctgtctcacgacgacacccacagcaggatcgagcactacgcatcacggctggcagagatggagaactccaacgggtcctacctgaacgacagtatcagccccaacgaatccatcgacgacgagcacctgctcatccaacactactgccagtccctcaaccaggactctcctctgtctcagccccggtcaccagcacaaatcctgatctctctggagagtgaggagagaggcgagctcgagcgcatcctggccgatcttgaagaagagaaccggaacctccaggccgagtacgacaggctcaaacagcagcacgagcacaaggggctcagtcccttgccctcacctccagagatgatgcccacctctccacagtcaccccgggacgctgaactgatcgcagaggctaagctgctgcggcaacacaagggacggctggaagctcggatgcagattctggaggaccacaacaagcagctcgaatcccagctccatcgcttgcgccagctcctggagcaaccccaagccgaggccaaggtcaacgggaccaccgtgtcctcacccagtacatccctgcagcggtccgacagcagccagcctatgctcctccgggttgtggggtcccagacctccgattccatgggtgaggaggaccttctctcaccaccacaggacacctcaaccggcctcgaggaggtgatggagcaactgaacaactcttttccttcctcacgcggccggaataccccaggcaagcctatgcgcgaggacacaatgtaa

SEQ ID NO: 10 - protein, 3685 aa, Homo sapiens; identical to SEQ ID NO: 2

SEQ ID NO: 11 - DNA, 11055 bp, Homo sapiens
  source 1..11055: mol_type = other DNA
atgctgtggtgggaggaggttgaggattgctacgaaagggaggatgtccaaaagaagaccttcaccaaatgggtgaacgcccagttcagcaagttcggtaagcagcacattgagaacctctttagcgacctccaggacggacgccgcttgctggatttgctggagggtctgaccgggcagaagctgcccaaggaaaagggctcaaccagggtccacgccctcaacaacgtgaacaaggccctgagggtgctgcagaacaataacgtcgacctggttaacatcggctccaccgacatcgtcgatggcaatcacaagctcaccctcggactgatttggaacatcatcctccactggcaggtgaagaacgtcatgaagaacatcatggccggcctgcaacagaccaactccgagaagatcttgctgagctgggtccggcagtccactcggaactatccccaggtcaacgtcatcaacttcaccaccagctggtccgacggactcgccctgaacgccctcatccactctcaccggccagacctgttcgactggaacagcgtcgtgtgccagcaaagcgcaacccagaggctcgaacacgccttcaacatcgccaggtaccagctcgggatcgagaagctcctcgaccccgaagacgtcgacaccacctaccccgacaagaagtccatccttatgtacatcactagcctgttccaggttctgccccaacaagtgagcatcgaggctatccaggaagtcgagatgttgcccagaccaccaaaggtgacaaaagaggagcacttccaactgcatcaccagatgcactactcccagcagatcacagtgagcctggctcagggttacgagaggacctcaagcccaaagccccggttcaagtcctacgcatacacccaggccgcttacgtcaccacctccgatcccaccaggtcaccattcccatcccagcatctcgaggcacccgaggataagtctttcggctccagtctcatggagtccgaggtcaatctggaccggtaccaaacagcactcgaggaagtgctctcttggctcctgtccgcagaagataccctccaggctcagggtgagatttccaacgacgtcgaggtggtgaaggaccagttccacacccacgagggctacatgatggacctcacagctcaccaggggagagtgggtaacattctgcaacttggctccaaactcatcgggaccggcaagctgagcgaggatgaagagaccgaggtccaggagcagatgaatttgctgaactcccggtgggagtgtctgcgggtcgctagcatggagaagcagtctaaccttcaccgggtcctcatggacctgcagaaccagaaactcaaagagctcaacgactggctgaccaagaccgaggagaggacacgcaagatggaggaagaaccactgggccccgatcttgaggacttgaagcggcaggtgcaacagcataaagtgctccaggaggacctggagcaagagcaggtgcgggtgaactcactcacccacatggtcgtcgtcgtggacgagtcttccggcgaccatgctaccgctgccctggaggaacagctgaaggtgcttggggacagatgggcaaacatctgcaggtggaccgaggatcggtgggttctcttgcaggacattctgctgaagtggcagcgcctgaccgaagagcaatgcctcttcagtgcctggctgtccgaaaaggaggacgctgtgaacaagatccataccaccggcttcaaggatcagaatgagatgctgtcatctctgcaaaagctggccgtcctcaaggccgatctcgagaagaagaagcaatccatgggcaagctgtacagcctgaagcaggatctgctgtccacactcaagaacaagagcgttacccaaaagaccgaggcctggctcgacaacttcgctcggtgctgggacaacctggtccagaaacttgagaagtccaccgcccaaatcagccaggccgtgaccaccacccagccctctctcacccaaaccaccgtcatggagacagtgaccacagtgactacccgggagcagatccttgtcaaacatgctcaggaggagcttccaccacctccaccccaaaagaagcggcagatcaccgtggatagcgagatccgcaagcggcttgacgtcgacatcaccgagttgcactcttggatcacccggtcagaggctgtcctgcagagcccagagttcgccatcttccgcaaggagggcaacttctccgacttgaaagagaaggtgaacgctatcgagcgggagaaggctgagaagttccggaagctgcaggacgctagccggtccgctcaggccctcgtcgagcaaatggtcaatgagggcgtcaacgcagactccatcaagcaggcatccgaacagctcaactctcggtggatcgagttctgccagctcctctccgagcggctgaactggctcgagtaccagaacaacattatcgccttctacaatcaactccaacagctcgagcagatgacaaccaccgctgagaactggctcaagatccagccaaccacaccaagtgagcccaccgccatcaagtcacagttgaagatctgcaaggacgaggtgaacagactgtccgatcttcagccacagatcgagagattgaagatccaatccatcgcacttaaagagaagggccaagggcccatgttcctcgacgccgactttgtggctttcaccaaccacttcaagcaggtgttttccgacgttcaagccagggagaaagagttgcagaccatcttcgacaccctccctcccatgcggtaccaagagaccatgagcgctatccgcacctgggtgcagcaatccgagaccaagctgagcatcccacaactctccgtcaccgactacgagatcatggagcagagactcggcgagctccaggctctgcagtcctctctgcaggaacaacagtccgggctgtattacctctctaccacagtgaaggagatgtccaagaaggccccatccgagatcagcaggaagtaccaatccgagttcgaagagatcgagggccggtggaagaagctctcctcccagctggtcgagcactgccagaagctcgaggagcaaatgaacaagctccggaagatccagaaccacatccagactttgaagaagtggatggctgaggtggacgtgttcctgaaggaagaatggcccgcactcggagattccgagatcctcaagaaacagctgaaacagtgccggctcctcgtgtccgatatccaaaccatccagccaagcctgaactccgttaacgagggtggccagaaaatcaagaacgaggctgagcccgagttcgcctctcggctcgagactgagctgaaagagttgaatacccagtgggaccacatgtgccagcaggtgtacgctcggaaggaggctcttaagggcgggctggagaagaccgtcagcctccaaaaggaccttagcgagatgcacgagtggatgacccaagctgaggaggagtacctcgagcgcgacttcgagtacaagacccctgacgagctccagaaggccgtggaggagatgaaacgcgcaaaggaagaggcccagcagaaggaagctaaagtcaagctcctcaccgagtccgtgaattccgtcatcgcccaagcaccacccgtggcccaagaagccctcaagaaggagctcgagaccctcactaccaactaccagtggctgtgcacccggctgaacggcaagtgtaagacactggaagaagtgtgggcctgctggcatgaactcctcagctacctggagaaggctaacaagtggctcaacgaggtggagttcaagctgaagaccaccgagaacattcccggtggggccgaagaaatctccgaggtcctggacagcctggagaacctgatgcggcattccgaggacaacccaaaccaaatccggatcctggcacagacactgaccgacggaggcgtcatggacgagctcatcaacgaagagctggagacatttaactccagatggcgggaactccatgaggaggccgttagacggcaaaagcttctggagcaatccatccagagcgcccaggagactgagaagagtctgcacttgatccaggagagtctgaccttcatcgacaagcaactcgccgcctacatcgctgacaaggtggacgccgcccaaatgccccaggaggctcaaaagattcagtcagacctgaccagccacgagatcagcctcgaagaaatgaagaagcacaaccagggaaaggaggcagcccagagggttctgtctcagatcgatgtggcacagaagaagcttcaggacgtgagcatgaagttccggctgttccaaaagcccgccaacttcgagcagcgcctccaagagtccaagatgattctcgacgaggtcaagatgcacctgcccgccttggagaccaagtctgttgaacaggaggtggtccagagccagctcaaccattgcgtcaacctttacaagtccctctcagaggtcaagagcgaagtggagatggttatcaagactgggcgccagatcgttcaaaagaagcagaccgagaaccctaaggaattggatgaacgggtcaccgccctcaagctgcattacaacgagctcggggccaaggtcaccgagagaaagcagcaattggagaagtgcctcaagctgtcccggaagatgcgcaaggagatgaacgttctgaccgagtggctcgcagccaccgacatggagctcaccaagcgctccgctgttgagggcatgcccagcaacctggattccgaagtggcttggggcaaggctacccagaaggaaatcgagaagcagaaggtgcatctgaagtccattaccgaagtcggcgaggcactcaaaaccgtcctcggcaagaaggagaccctggtcgaagacaagttgtcccttctgaactccaactggatcgctgtcacctcacgggccgaagagtggctgaatcttctgctggagtatcagaaacacatggagaccttcgaccagaacgtcgatcacattaccaagtggatcatccaggccgatacattgctggatgagtccgagaagaagaagccccagcagaaagaggatgtgctgaagcggcttaaggccgagttgaacgacatccggcccaaggtcgactcaactcgcgaccaggccgcaaacctgatggccaatcggggcgatcactgtcggaagctggtggaaccccagatctcagagctgaatcacaggttcgccgccatttcccacagaatcaagacaggcaaggcaagcattcctctcaaggaactggagcagttcaacagcgacatccagaaactccttgaaccactggaggctgagatccagcagggtgtcaatctgaaagaagaggatttcaacaaggacatgaacgaggacaacgaggggaccgtcaaggagcttctgcagcgcggtgacaacctccagcaacggatcaccgatgagagaaagcgggaagagatcaaaatcaagcagcagctcctgcagaccaagcacaatgctctgaaggacctgagatcccagcggcggaagaaggctctggaaatctcccaccaatggtaccagtacaaacggcaggctgacgatctgctcaagtgcctggacgacatcgaaaagaagttggcctccctgccagagccaagggacgagagaaagatcaaagagatcgaccgggagctgcaaaagaagaaggaagagctgaatgcagtgaggcggcaagccgagggtctgtccgaagacggggctgcaatggctgtcgagcccacccagattcagctgtccaagcggtggcgggagatcgaatctaagttcgcccagttccgcagactcaacttcgcccagatccacaccgtgagagaggagaccatgatggtgatgaccgaggacatgcccctggagatcagttacgtgccctccacctacctgaccgagatcacccacgtgtcacaggccctgctggaggtcgagcagctcctcaatgctccagacctctgcgccaaggatttcgaggatctgttcaagcaggaggagtccctgaagaatatcaaggacagcctgcagcagagtagcgggcggatcgacatcatccacagtaagaagaccgctgcactgcaatccgcaaccccagtggagcgggtcaagttgcaagaggcactcagccagctggacttccagtgggagaaggtcaataaaatgtacaaagaccggcagggcaggttcgaccggtccgtggagaagtggcggcggttccattacgacatcaagatctttaaccagtggctcaccgaagccgagcaattcctgaggaagacccagatcccagagaactgggagcacgccaagtacaagtggtacctgaaggaattgcaggatggtatcggccaacggcagacagtcgtgcggacactcaacgctaccggggaggagattatccagcagagcagcaagaccgacgcctccattctccaggagaagcttggttccctgaacctgcggtggcaggaggtctgcaagcagctcagcgacaggaagaagcggctggaagagcagaagaacatcctgtccgagttccaacgggatctcaacgagttcgtcctgtggctggaggaagctgacaacatcgcttccatccctctggagcccggcaaagaacagcagctcaaggagaagctggagcaggtgaaactcctggtggaggaacttcccctgcggcagggtatcctcaagcagctgaatgaaactggtgggcccgtgcttgtctccgcacccatttcccctgaggagcaggacaaactcgagaacaagttgaagcagaccaacctgcagtggatcaaggtctcccgggctcttcccgagaaacagggcgagattgaggcccagatcaaggatctcggccagcttgagaagaagctggaagatcttgaagagcagttgaaccatctcttgctgtggctgtccccaatccggaaccagctggaaatctacaaccagccaaaccaggaggggcccttcgacgtgaaggaaactgagatcgcagtgcaggccaagcagccagacgtcgaggagatcctgtccaaggggcaacacctctacaaagaaaagcctgcaactcagcctgtcaagcggaaattggaggacctctccagtgagtggaaggccgtcaaccggctcctgcaagaattgcgcgccaaacaacccgacttggcaccaggcctgactacaatcggcgcatcacccacccagaccgtgaccctggtgacccagccagtggtgactaaagagaccgccatctccaagctggagatgccttcctccctcatgctcgaggtgccagcccttgcagacttcaaccgggcatggacagagctcactgattggctgtccctcctcgaccaggtcatcaagagccaacgggtcatggttggggacctggaagatatcaacgagatgatcatcaagcagaaggctaccatgcaagatctcgagcaacggcggccacagctcgaagagctcatcactgccgcccagaacctcaagaataagacctccaaccaggaagcccggactatcatcacagaccgcatcgagcgcatccaaaatcagtgggatgaggtgcaggagcacctccagaatcggcggcaacagctgaacgaaatgctcaaggattccacccagtggcttgaggctaaagaggaggcagagcaagtcctgggccaagcaagggcaaagctggagtcctggaaggaaggcccctacaccgtcgacgctatccagaagaagatcaccgagaccaaacagctggcaaaggacttgaggcagtggcagaccaatgtcgacgtggccaacgacctcgctcttaagctgctgcgggactactccgctgacgatacccggaaggtccacatgatcaccgaaaatatcaacgcttcctggcggagcatccataagcgggtgtcagagcgcgaggcagctctggaggagacccatcggctgctgcaacagttccctctggacctcgaaaagttcctggcttggttgacagaagccgagaccaccgctaacgttctgcaggatgccacccgcaaggagagattgctggaggactcaaagggcgtgaaggagctcatgaagcagtggcaggaccttcagggcgaaatcgaagctcacacagacgtctaccacaacctcgacgagaactcccaaaagatcctgcgctccctcgaaggcagcgacgacgctgtgcttctgcagaggaggctggacaacatgaacttcaagtggtccgagctgcgcaagaagtccctgaacatccggtcccatttggaagcttcctccgaccagtggaagcggctgcatctctccctgcaggaactgctggtgtggctccaactcaaggacgatgaactgagccggcaggcaccaatcggaggggatttccccgccgtccagaagcagaacgatgtgcaccgggctttcaagcgggagctcaagaccaaggagccagtgatcatgagcaccctggagaccgtgcggatcttcctgacagagcagccactggaaggcctcgagaagttgtatcaggagcctcgggaactcccacctgaagagcgcgcccagaacgtgactcggctgctccgcaagcaggcagaggaggtcaacaccgaatgggagaagctcaacctgcattccgccgactggcagaggaagatcgatgaaacactggagcggctccaggagctccaagaggcaaccgacgagctggatctgaaactgagacaggccgaagtcatcaagggaagctggcaacccgttggggacttgctcatcgactccctgcaagaccatctggagaaagtgaaggcactgagaggcgagatcgcaccactgaaggagaacgtctcccacgtgaatgatcttgcccgccagctgacaaccctgggcatccaactctccccatacaacctcagcaccctcgaggacctgaacacccgctggaaacttctgcaagtggcagtcgaggacagggtcaggcagctccatgaggctcaccgcgactttggacctgccagccagcacttcctgtctacctccgtccaaggcccatgggagagagccatctctcctaacaaggtgccctactacatcaaccacgagactcagaccacctgctgggatcaccccaagatgaccgaactgtatcagtccctggctgatctgaacaatgtccgcttctccgcataccgcacagccatgaagcttagacggctgcagaaggcactctgcctcgacctgctctctctgtcagcagcctgcgatgcactcgaccagcacaacctcaagcagaatgaccagcctatggatatcctgcagatcatcaactgcctgaccaccatctacgatcggctcgaacaggagcacaacaatctggtgaacgtgcctctgtgcgtcgacatgtgcctcaattggctcctgaacgtgtatgacaccgggaggaccggaaggatccgggtgctcagcttcaagacagggatcatcagcctctgcaaggcccatcttgaggataaataccgctacctcttcaaacaagtcgccagctctaccgggttctgcgaccaacgccgcttggggcttctgctccacgattcaattcaaatcccacggcagctcggtgaggtggcttccttcggcggctccaacatcgagccttctgtccgcagctgcttccagttcgccaacaacaaacctgagatcgaggcagccctgttcctggactggatgcggctcgagcctcaatccatggtctggctgcccgtgctccatcgggtcgctgctgcagagacagccaagcaccaggccaagtgcaacatctgtaaagagtgcccaatcattggcttccggtacagatccctcaagcacttcaactatgatatttgccagagctgcttcttctccggccgggttgcaaagggccacaagatgcactatcctatggtggagtactgcaccccaaccacctccggtgaagacgtgcgggatttcgccaaggtccttaagaacaagttccggaccaaacggtacttcgccaaacacccacggatggggtacctgcccgtgcagaccgtgctcgaaggggataacatggaaacccctgtcaccctgatcaatttctggccagtggactctgctccagccagcagcccacagctctcacacgacgacacccactcccggatcgagcactatgctagccggctggctgagatggagaactccaacggctcctacctgaacgactcaatcagccccaacgagagcatcgacgacgagcacctgctgatccagcactactgccagtcactgaaccaggactcacccctctctcagcctaggtccccagcccagatcctcatctctctggagtctgaggagaggggcgagctcgagaggatccttgccgacctggaggaggagaaccggaacctgcaggctgagtacgacaggctgaaacagcagcacgagcacaaggggctgtctccactcccttcacctcctgagatgatgcccacaagccctcagagccctagagacgccgagctgatcgccgaggccaagctgctgagacagcacaagggcaggctggaagctcggatgcagatcttggaagaccacaacaagcagctggagtctcagctccacagactgcggcagcttcttgagcagccacaggctgaggccaaggtgaacggcaccaccgtcagctctcccagcacctccctgcaacggtctgattccagccagcccatgctgctccgggttgtgggctcccagacctccgactccatgggcgaggaagacttgctgtcaccaccacaggataccagcaccggtctcgaagaggtgatggaacaactgaacaactccttcccctcttcccggggccggaacacacccggtaagcccatgcgcgaggacaccatg

SEQ ID NO: 12 - protein, 3685 aa, Homo sapiens; identical to SEQ ID NO: 2

SEQ ID NO: 13 - DNA, 11055 bp, Homo sapiens
  source 1..11055: mol_type = other DNA
atgctgtggtgggaggaggtcgaggactgttacgagcgtgaggacgtccaaaagaagacctttaccaagtgggtgaacgcccagttcagcaagttcggcaagcagcacatcgagaatctgttctccgacttacaagacggccggcggctacttgacctactggagggactgaccggccaaaagctgcccaaggagaaggggagcactcgggtgcacgctctgaacaacgtcaacaaggccctgcgtgtgctgcagaacaacaacgtggacttagtgaacatcggcagcaccgacatcgtggacggcaatcacaagctgaccttaggactgatctggaacatcatcctgcactggcaggtgaagaacgtgatgaagaatatcatggccggactgcagcagaccaatagcgagaagatactcctgagctgggtgcgtcaaagcacccgtaactaccctcaggtgaacgtcatcaactttactactagctggagcgacggccttgccttaaacgccctgatccactcccaccggcctgacctgttcgactggaacagcgtggtgtgtcaacagtccgccacacagaggctggagcacgccttcaacatcgccaggtaccagctgggcatcgagaagctactggaccccgaggacgtcgacaccacctaccccgacaagaagagcatcttaatgtacatcaccagcctgttccaagtgctgccacagcaggtgagcatcgaggccatccaagaggtcgagatgttaccccggccacctaaggtgactaaggaggagcacttccagctgcaccaccagatgcactacagccagcagataaccgtgtcccttgcccagggctacgagaggactagcagcccaaagccacggttcaagagctacgcctacacacaggccgcctacgtgacaaccagcgatcccaccaggagccccttcccctcacagcacctggaggcacccgaggacaagagcttcggctcatccctgatggagtcagaggtgaacctggaccggtaccagacagcccttgaggaggtcctgtcatggctactgagcgccgaggacactctgcaggcccagggtgagataagcaatgacgtggaggtggtgaaggaccagttccacacccacgagggctacatgatggacctaacagctcaccagggtcgggtcggaaacatccttcaactgggctcaaagctgatcggcaccggcaagcttagcgaggacgaggagaccgaggtccaagagcaaatgaatctgctgaactcaaggtgggagtgcctgcgtgtggcctccatggagaagcagtcaaaccttcacagggtgcttatggacctgcaaaaccagaagctcaaagagcttaatgactggctgaccaaaactgaggagcggaccaggaagatggaggaggagcccctaggacctgatcttgaggatttaaagcggcaggtccaacagcacaaggtgttacaagaggatttagagcaagagcaggtgcgggtgaactccctgacccacatggtggtggtggtggatgagtcaagcggagaccacgctactgccgcccttgaggagcaactcaaagtgttaggcgaccggtgggctaacatctgccggtggacagaggaccggtgggtgctactgcaagatattctgctgaagtggcagcggttaacagaggagcagtgcctgttttccgcctggctgagcgagaaggaggatgccgtgaacaaaatccacaccaccggcttcaaggaccaaaatgagatgctctcatccttacagaagctagcagtgctaaaggccgacttagagaagaagaagcaaagcatgggcaagctgtacagcctcaagcaggacttacttagcacactcaagaataagagcgtgacccagaagaccgaggcctggttagacaacttcgcccggtgctgggacaaccttgtgcagaaactcgagaagagcaccgcccaaatttcacaggccgtgactaccacccagccaagcctcacccagaccaccgtcatggagactgtcaccactgtgaccacccgggagcagatcctcgtgaagcacgcccaagaggagctgccacctcctccaccccagaagaagcggcagatcaccgtcgactccgagatccgtaagcggctggatgtggacatcaccgagctgcacagctggatcaccaggtccgaggccgtcctacaaagccccgagtttgccatctttcggaaggagggtaactttagcgacctcaaagagaaagtgaacgccatcgagcgggagaaggccgagaagttccggaagctacaggacgcctcacggtcagcccaggctctggtcgagcaaatggtgaacgagggagtgaacgccgactcaatcaagcaggccagcgagcagctgaattcacggtggatcgagttttgtcagctgctgtcagagaggctcaactggctcgagtaccaaaacaacatcatcgccttctacaaccaacttcagcagctcgagcagatgaccaccaccgctgagaactggctgaaaatccagccaaccactccaagcgagcctaccgccatcaagtcacagctgaagatttgcaaagacgaggtgaaccggctgagcgatctgcaaccccagatcgagcggctcaagatccagagcattgccttaaaggagaaaggccaaggccccatgttcctagacgccgacttcgtggccttcaccaaccactttaagcaagtgttcagcgatgtgcaagcccgggagaaggagctacagaccatcttcgataccctgcctcccatgcggtaccaggagacaatgagcgccatccggacatgggtccagcagtcagagaccaagctgagcattccacaactgagcgtgaccgactacgagatcatggagcagaggttaggcgagctgcaggctcttcagagcagcctgcaagagcaacaaagcggcctgtactacctgagcaccaccgtcaaggagatgagcaagaaggccccttccgagattagccggaagtaccagagcgagttcgaggagatcgagggccggtggaagaagctgtccagccagctagtggagcactgccagaagcttgaggagcaaatgaacaaactgcggaaaatccagaatcacattcagaccctgaagaagtggatggccgaggtggacgtgttccttaaggaggagtggcccgctcttggcgacagcgagatactcaagaagcaacttaaacagtgccggttactggtgagcgacatacaaaccatccagcctagcctgaacagcgtcaacgagggtggccagaaaatcaagaacgaggctgagcccgagttcgcaagccggctggagaccgagcttaaagagctcaacacccagtgggaccacatgtgtcagcaggtgtacgcccgtaaagaggcacttaaaggcggactggagaagactgtgtcactgcagaaggatctgagcgagatgcacgagtggatgacccaagctgaggaggagtacctggagagggacttcgagtacaagaccccagacgagctgcagaaagccgtcgaggagatgaaacgggccaaagaggaggctcagcagaaagaggcaaaagtgaagctactgaccgagagcgtgaatagcgtgatcgcccaagctccacccgtggctcaggaggcactgaagaaggagttagagacactgaccaccaactaccaatggctttgcacccggctgaacggcaagtgcaagacactagaggaggtgtgggcatgctggcacgagctgctgagctacctcgagaaagccaacaagtggctgaatgaggtggagttcaagctaaagaccaccgagaatatacctggtggcgcagaggagatcagcgaggtgctggacagcctagagaacctgatgcggcacagcgaggacaaccccaaccagatccgtatcctggcccagaccctaactgatggcggcgtgatggacgagctgatcaacgaggagctcgagaccttcaatagcaggtggcgggagttacacgaggaggccgtgcgtcggcaaaagctacttgagcagagcatccaaagcgcccaggagacagagaagtccctgcacctgatccaggagtcactcaccttcatcgacaagcagctagccgcctacatcgccgacaaggtggacgcagcccagatgccccaggaggcacagaagatccagtcagacctgaccagccacgagatcagccttgaggagatgaagaagcacaaccagggcaaagaggcagcccaacgggtgctaagccagatcgacgtggcccagaagaaactgcaggacgtgagcatgaagttccggctgttccaaaagcccgccaacttcgagcagcggcttcaggagagcaagatgatcttagatgaggtcaagatgcacctgccagccttagagaccaagtcagtggagcaagaggtggtgcagagccagcttaaccactgtgtgaacctttacaagagccttagcgaggtcaaaagcgaggtggagatggtgataaagacaggccggcagatcgtgcaaaagaagcagaccgagaaccccaaggagcttgacgagcgggtcactgcccttaagttacactacaacgagctgggcgccaaggtcaccgagcggaaacagcagctagagaaatgcctgaagctctcccggaagatgcggaaagagatgaacgtgctaaccgagtggctggcagccaccgacatggagctgaccaagaggtcagctgtggagggcatgcccagcaacctcgacagcgaggtcgcatggggcaaggccacccagaaagagatagagaagcagaaggtgcacctgaagagcatcaccgaggtgggcgaggccctaaagactgtgctgggcaagaaggagacattagtcgaggacaaactgagccttctgaacagcaactggatcgcagtgactagccgggcagaggagtggttaaacctgctactggagtaccagaagcacatggagacttttgaccagaacgtggaccacatcaccaagtggatcatccaggctgacaccctgctcgacgagagcgagaagaagaagccccaacagaaagaggatgtgctgaagcggctgaaggcagagctgaacgatatccggcccaaggtcgatagcacccgtgaccaggcagccaacctgatggccaaccggggcgatcactgtcggaagctggtggagccccaaatcagcgagctcaaccaccggttcgctgccatcagccaccggatcaagacaggcaaggccagcattccattaaaggagctagagcaattcaactccgacatccagaaactgctagagcccctagaggctgagatccaacagggcgtgaatctgaaggaggaggacttcaacaaggacatgaacgaggacaacgagggcacagtcaaggagctgctgcaacggggcgacaacctgcaacagcggatcaccgacgagaggaagcgtgaggagatcaagatcaagcaacagcttctgcaaaccaagcacaatgcccttaaggacctacggagccaacggcggaagaaggccctggagatcagccaccagtggtaccagtacaagcggcaggcagacgacctactgaagtgcctggacgacattgagaagaagttagccagcctgcctgagccaagggacgagcggaaaatcaaggagatcgaccgggagcttcagaagaagaaggaggagcttaacgcagtccggcggcaggccgagggtctctcagaggatggcgctgctatggcagtggagcccacccagatccaactgagcaagcgttggcgtgagatagagagcaagttcgcccagttccgtcggctgaacttcgcccagatccacacagtgagggaggagactatgatggtgatgactgaggacatgcccctggagattagctacgtgcccagcacctacctgaccgagatcacacacgtgtcccaggcccttctggaggtggagcagctcttaaacgctcccgacctgtgtgccaaggacttcgaggacctgtttaagcaggaggagagccttaagaacatcaaggactccttacagcaaagcagcggccggatcgacatcatccacagcaagaagaccgctgccctacagagcgccacacctgtggagcgtgtcaaactacaagaggcactgagccaactggacttccaatgggagaaggtcaataagatgtacaaggaccgtcagggcaggttcgaccggagcgtggagaagtggcggcggttccactacgacattaagatcttcaaccagtggttaactgaggcagagcagttcctgcggaagacccagatcccagagaactgggagcacgccaagtacaagtggtacctgaaggagctgcaagacggcataggtcagcgtcagaccgtcgtccggaccctaaacgccaccggcgaggagatcatccaacagagcagcaagaccgacgccagcattctgcaggagaaactgggcagcctgaacctgcggtggcaggaggtgtgcaagcagctaagcgaccggaagaaaaggcttgaggagcaaaagaacatcctgagcgagttccagcgggacctgaacgagttcgtgctgtggctagaggaggccgacaacatcgcctcaatacccctggagcccggcaaagagcaacagctcaaggagaaacttgagcaggtcaagctgctagtggaggagcttccactgcggcagggcatcttaaagcagctaaacgagactggtggccccgtcttagtgagcgcccctatcagccctgaggagcaggataagctagagaacaagctgaagcaaaccaatctgcagtggatcaaagtgtcacgggccctgccagagaagcaaggggagatagaggctcagatcaaagacctgggtcagctcgagaagaagttagaggacctagaggagcagctaaatcacctcttactgtggctgagcccaatccggaaccaactggagatctacaatcagcctaaccaagagggtcccttcgacgtgaaagagacagagatcgccgtgcaagccaaacaacccgatgtggaggagatcctgagcaagggccaacacctgtacaaagagaagccagcaacccaacccgtgaagcggaaactggaggacctcagcagcgagtggaaggccgtgaacaggttactgcaggagctgcgtgccaagcagcctgacctagcacccggcctgactaccataggcgccagccctacccaaactgtgaccctagtgacccagcccgtcgtgaccaaggagaccgccattagcaaactggagatgccatcaagcctgatgctggaggtgcccgccttagccgacttcaatcgggcctggaccgagctcacagactggctgtccctgctggaccaggtcatcaagagccaacgggtgatggtgggagatctggaggacatcaacgagatgatcatcaagcagaaggccaccatgcaagacctagagcagcgtcggccccaattagaggagctgatcaccgcagcacaaaacctgaagaacaagaccagcaatcaggaggctcggaccatcatcaccgaccggattgagcggattcagaaccagtgggatgaggtgcaagagcaccttcagaaccgtcggcaacagcttaacgagatgctgaaggacagcacccaatggcttgaggcaaaggaggaggctgagcaagtcctcggtcaggctagggccaagcttgagagctggaaagaggggccctacaccgtggacgccatccagaagaagatcactgagaccaaacagctggccaaagacctgcggcagtggcagaccaacgtggacgtggccaacgacctggcattaaagttacttcgggactacagcgcagacgacacacggaaggtgcacatgattaccgagaacatcaacgcctcatggcggagcatccacaagcgggtgagcgagagggaggctgctcttgaggagacccaccggctcctgcaacagttcccactggaccttgagaaattcctggcatggctgaccgaggcagagaccaccgccaacgtgctccaagacgccacccgtaaggagcggctactggaggacagcaagggagtgaaagagctaatgaagcagtggcaggacctccaaggcgagatcgaggcccacaccgacgtgtaccacaacctggacgagaacagccagaagattctccggagcctggagggatccgacgacgccgtcctcctgcagaggaggctggacaacatgaacttcaagtggagcgagctgcggaagaagagcctcaacatccggagccacctagaggccagcagcgaccagtggaaacggctgcacctgtcactgcaagagcttcttgtgtggctgcagctgaaagacgacgagctgtcacggcaggcacccataggtggcgatttccccgccgtgcagaagcagaatgacgtgcaccgggccttcaagcgggagctgaaaaccaaggagcccgtgatcatgagcaccctagagaccgtccggatcttcctgaccgagcagccccttgagggcctagagaaactgtaccaggagcctcgtgagctgcctccagaggagagggcccagaacgtgacccggttactgcggaagcaggcagaggaggtgaacaccgagtgggagaagctgaatctgcacagcgctgactggcagcggaagattgacgagaccctcgagcggctacaagagctccaggaggccaccgatgagctagacctaaagctgcgtcaggccgaggtgattaagggctcatggcagccagtcggcgatcttttaatcgactccctccaagaccacctagagaaggtgaaggctttacggggcgagatagcccctctaaaggagaacgtgagccacgtgaatgacctggcacggcagctgaccaccctgggcatccagctaagcccctacaacctgtcaacactcgaggatctaaacacccggtggaaactgctgcaggtcgcagtggaggacagggtccggcaactgcacgaggctcaccgggacttcggacctgctagccagcacttcctgtccactagcgtgcaaggcccttgggagcgggcaatttccccaaacaaggtgccctactacatcaaccacgagactcagacaacctgctgggaccaccctaagatgaccgagctgtaccaaagcctggccgacttaaacaacgtgcgtttctccgcttaccggaccgccatgaagttacgtcggctgcagaaggcactgtgcctagacctgctgtcactgtccgctgcctgtgatgccctagaccagcacaacctgaagcaaaacgaccagcccatggatatcctgcagatcatcaactgcttaaccaccatctacgaccgtctcgagcaggagcacaacaacctggtcaacgtgcccctgtgcgtggacatgtgcctgaactggctcctgaatgtgtacgacactggacgtactggacggatccgggtgctttcattcaaaaccggcatcatcagcctgtgcaaggctcaccttgaggacaaataccggtacctattcaagcaggtcgcctcaagcaccggcttctgcgaccagaggcggcttggactgctgctgcacgatagcatccagataccccgtcagttaggcgaggtggcaagcttcggtggcagcaacatcgagccctcagtgcggagctgcttccagttcgccaacaacaaaccagagattgaggcagccttattcctggactggatgcggcttgagccccagagcatggtgtggctccccgtgctacacagggtggcagctgcagagaccgccaaacaccaggccaagtgcaacatatgcaaggagtgccccatcatcggcttccgttaccggtcactgaagcacttcaactacgacatatgccagagctgcttcttctccggacgggtggcaaagggccacaaaatgcactaccccatggtggagtactgtaccccaactacaagcggtgaggacgtgcgggactttgccaaggtgctgaagaacaagttccggacaaagcggtacttcgctaagcacccacggatgggctacctgcccgtgcagaccgtcctggagggcgacaacatggagacacccgtcaccctcattaacttttggcccgtggattcagctccagccagcagccctcagctgagccacgacgacacacacagccgtatcgagcactacgctagccgtctagccgagatggagaacagcaacggcagctacctgaacgacagcataagccccaacgagagcatcgacgacgagcacttactgatccagcactactgccaaagcctgaaccaggacagccctctgtcacaacctcggagccccgctcaaattctgatcagcctagagtcagaggagcggggcgagttagagcggatactggccgatttagaggaggagaaccggaacctgcaggccgagtacgaccggttaaaacagcagcacgagcacaaggggttaagcccactacccagccctcccgagatgatgcccacctcacctcaatccccacgtgatgccgagctgatcgcagaggccaagctgctgcgtcagcacaaagggaggcttgaggccaggatgcagatccttgaggaccacaacaagcaactcgagagccaactgcaccggctgaggcaactgctggagcaaccccaagccgaggccaaagtgaacggcactaccgtctcatcacccagcacctccctgcagcgtagcgacagcagccagcccatgcttctgagggtggtgggcagccagaccagcgacagcatgggcgaggaggaccttctgtcaccaccccaggacactagcaccggactggaggaggtcatggagcaacttaacaacagcttcccttccagccgtggtcggaatacacccggcaagcctatgcgggaggacaccatg

SEQ ID NO: 14 - protein, 3685 aa, Homo sapiens; identical to SEQ ID NO: 2

SEQ ID NO: 15 - DNA, 11055 bp, Homo sapiens
  source 1..11055: mol_type = other DNA
atgctgtggtgggaggaggtggaggactgctacgagcgggaggatgtccagaagaagaccttcactaagtgggtgaacgcccagttcagcaagttcggcaagcagcacatcgagaatctattcagcgacctacaggacggccgtcggctactagaccttttagagggtctgaccgggcagaagctgcccaaggagaagggtagcaccagggtccacgccctcaacaacgtgaacaaggctctgcgggtgctgcagaacaacaacgtggaccttgtgaacatcggcagcaccgacatcgtggacggcaaccacaagctgacactgggcctgatctggaacatcatcctgcactggcaggtgaagaacgtgatgaagaacattatggccggcctgcaacagaccaacagcgagaagatactcctgagctgggtgcgtcagagcacccggaactaccctcaggtgaacgtcatcaacttcacaacctcatggtcagacggccttgccctaaacgccctgatccactcacaccggcccgacctgttcgactggaacagcgtggtgtgccaacagagcgccacacaacggctagagcacgccttcaatatcgccaggtaccagctgggcatcgagaagctgctcgaccccgaggatgtggacaccacctaccccgacaagaagagcatcctgatgtacataaccagcctgttccaggtgctaccccagcaggtcagcatcgaggccatccaagaggtcgagatgctcccacggcctccaaaggtgaccaaggaggagcacttccagctacaccaccagatgcactacagccagcagataaccgtgagcttagcccagggatacgagcgtaccagcagccctaagccccggttcaagagctacgcttacacccaggccgcctacgtcactaccagcgacccaactcggagcccattcccctcacagcacctagaggcacccgaggacaagagcttcggtagcagcctgatggagtcagaggtcaacctggacaggtaccagactgccctagaggaggtgctctcatggctactgtcagctgaggacaccctccaggcacagggagagattagcaacgacgtcgaggtggtgaaggaccagttccacacccacgagggctacatgatggacctaactgctcaccaggggcgtgtgggaaacatcctgcaactcggcagcaagctgatcggcaccggcaagctgagcgaggatgaggagaccgaggtgcaagagcagatgaacctactgaacagccggtgggagtgcctacgggtggcctccatggagaaacaaagcaacctacacagggtgctcatggatctgcagaaccagaagctgaaagagctcaatgattggctgaccaagactgaggagcggacacgtaagatggaggaggagccactgggtcccgacctggaggacctaaagcggcaggtgcaacagcacaaggtccttcaagaggatctggagcaagagcaggtgcgggtgaacagcttaacccacatggtcgtcgtggtggacgagtccagcggtgaccacgctaccgcagcattagaggagcagctaaaagtgctgggtgacaggtgggccaacatctgccggtggaccgaggataggtgggtgctgctgcaagacatcctactgaagtggcagcggctcacagaggagcagtgcctattcagcgcctggctgagcgagaaggaggatgccgtgaataaaatccacaccaccgggttcaaggaccagaatgagatgctgtcaagcttacagaaactggccgtgctgaaggccgatctggagaagaagaagcaaagcatgggcaagctgtactcactgaagcaagatctgctgtccacccttaagaacaagagcgtgacccagaagaccgaggcctggctcgacaacttcgcacggtgctgggacaacctagtgcagaaactagagaagagcacagcacagatcagccaggctgtgactacaacccaacctagcctgactcagactaccgtgatggagaccgtcaccacagtgaccacccgggagcagatccttgtgaagcacgcccaagaggagcttccacctccacctcctcagaagaagcgtcagatcaccgtcgactcagagatcaggaagcggctggacgtcgacatcaccgagctgcacagctggatcacccggtcagaggctgtgctgcaaagccccgagtttgccatcttccgtaaggagggcaacttcagcgatctaaaggagaaggtcaacgccatcgagcgggagaaggccgagaagttccggaagttacaggatgcctcacgttccgctcaggccctcgtggagcagatggtcaacgagggcgtgaatgccgatagcatcaagcaggccagcgagcaactgaatagccgttggatcgagttttgccaactgctatccgagcgtcttaactggctagagtaccaaaacaacatcatcgccttctacaaccaactacagcagcttgagcagatgaccaccaccgctgagaactggctgaagatacagccaaccactcctagcgagcccacagccatcaaaagccagctgaagatttgtaaggatgaggtgaacaggctaagcgatctgcaaccccagatcgagcggctcaagatccagagcatcgcattaaaggagaaaggccagggccccatgttcctggatgccgacttcgtggccttcaccaaccacttcaagcaggtctttagcgatgtgcaagcccgggagaaagagcttcagacaatcttcgacactctgcctcccatgcggtaccaggagacaatgagcgccatacggacctgggtccaacagtccgagaccaagctgagcataccccagctgagcgtgaccgactacgagatcatggagcagcggctgggtgagctgcaggcccttcagtccagccttcaggagcaacagtccggcctgtactacctcagcactaccgtcaaggagatgagcaagaaggcaccctccgagatcagccggaagtaccagagcgagttcgaggagattgagggccggtggaagaagttatcaagccagctagtggagcactgccaaaagctggaggagcagatgaataaactgcggaagatccagaatcacatccagactctgaagaagtggatggccgaggtggacgtgttcttaaaggaggagtggcccgccttaggcgactccgagatcttaaagaagcaactcaagcagtgtcggctcctggtgagcgacattcagaccatccagcctagcctgaatagcgtgaacgagggtggccagaaaatcaagaatgaggccgagcctgagttcgcatcccggctggagacagagttaaaggagcttaacacccagtgggaccacatgtgtcagcaggtgtacgcacggaaagaggccctgaaaggcggccttgagaagaccgtcagcctccagaaggatctgagcgagatgcacgagtggatgacccaagctgaggaggagtacctggagcgtgacttcgagtacaagaccccagatgagctccagaaagcagtcgaggagatgaaacgggcaaaggaggaggctcagcagaaggaggccaaggtcaagcttctgaccgagagcgtgaactccgtgatcgctcaggctcctcccgtcgctcaggaggctctcaagaaggagctagagacactgaccaccaactaccagtggctatgcactcggctgaacggcaagtgcaagacactcgaggaggtgtgggcttgttggcacgagctgctgagctacctagagaaggccaacaagtggctgaatgaggtggagttcaagcttaagacaaccgagaacatccctggtggcgcagaggagataagcgaggtgctggacagcttagagaacctgatgcggcacagcgaggacaaccccaaccagatccggatccttgcccagaccttaaccgacggtggcgtgatggacgagctgatcaacgaggagcttgagaccttcaacagccgttggcgtgagcttcacgaggaggctgtccgtcggcaaaagttactagagcagagcatccagagcgcccaagagaccgagaagtcactgcacctgatccaggagagcttaaccttcatcgacaagcaactcgccgcctacatcgccgacaaggtggacgccgcccaaatgccacaagaggcacagaaaattcagagcgacctgaccagccacgagattagccttgaggagatgaagaagcacaaccagggcaaagaggccgcccagcgtgtcctgagccagatcgacgtggcacaaaagaagctccaggacgtgagcatgaagttccggctgttccagaagcccgccaacttcgagcaacggcttcaggagagcaagatgatcttagacgaggtcaagatgcacctgccagctctggagaccaagagcgtcgagcaggaggtggtgcaaagccaactgaatcactgtgtgaatctgtacaaaagcctttcagaggtgaagagcgaggtggagatggtgataaagaccggccggcagattgtccaaaagaagcagaccgagaaccccaaggagcttgacgagcgtgtgaccgctctgaagctgcactacaacgagctgggcgccaaggtcacagagcggaagcagcagctcgagaagtgcctgaaactgagccggaagatgcgtaaggagatgaacgtgttaaccgagtggctggcagccaccgacatggagctaaccaagcgttccgcagtggagggcatgcccagcaacctggactccgaggtcgcatggggaaaggccacccagaaggagatagagaagcagaaggtgcacctgaagagcataaccgaggtcggcgaggctttaaagacagtgctgggcaagaaggagacccttgtcgaggataagctgtccctgctgaatagcaactggatcgctgtgaccagcagggctgaggagtggcttaacctgctcctggagtaccaaaagcacatggagacattcgaccagaacgtggaccacatcaccaaatggatcatccaggccgataccctgctagacgagtccgagaagaagaagccccaacagaaagaggatgtcctgaagcggttaaaggccgagttaaatgacatccggcccaaggtggactcaacccgtgatcaggccgccaacctgatggccaaccggggcgatcactgcaggaagctggtggagccccaaattagcgagttaaaccaccggttcgccgccattagccaccggatcaagaccggtaaggccagcattccccttaaagagctagagcagttcaacagcgacatacagaaacttcttgagcccctagaggccgagatccagcaaggggtgaatctgaaagaggaggacttcaacaaggacatgaacgaggacaacgagggcaccgtcaaagagttactccagcggggcgacaacctgcaacagcggatcaccgatgagcgtaagcgtgaggagatcaagatcaagcagcagttactacagaccaagcacaacgccctaaaggacctgaggtcacagcggcggaagaaggctctggagatcagccaccagtggtaccagtacaagcgtcaggccgacgacctcctgaagtgcctggacgacatcgagaagaagctagcaagcctgcctgagccacgtgacgagcggaaaattaaggagatcgaccgggagctacagaagaagaaggaggagctcaacgccgtgaggcgtcaagctgagggactgtccgaggatggcgctgcaatggccgtggagcctacccagatccaactgagcaagaggtggcgggagattgagagcaagttcgctcagttccggcggttaaacttcgcccagatacacaccgtccgggaggagactatgatggtgatgactgaggacatgccccttgagatcagctacgtgcccagcacctacctgaccgagatcacccacgtgagccaggcactactggaggtggagcaactactgaacgcaccagacctgtgcgccaaggacttcgaggacttatttaagcaggaggagtcactaaagaacatcaaggactcacttcaacagagcagcggccggatcgacatcatccacagcaagaagaccgctgccctgcaaagcgccaccccagtggagagggtgaagctgcaagaggcactgtcccagctggacttccaatgggagaaagtcaacaagatgtacaaggaccggcaaggcaggttcgaccggagcgtggagaagtggcggcggttccactacgacatcaagatcttcaaccaatggctaacagaggcagagcaattcctgcggaagacccagatcccagagaattgggagcacgccaagtacaagtggtacctaaaagagctgcaagatggcattggccagaggcagaccgtggtgcggacccttaacgccaccggcgaggagattatccagcagtccagcaagaccgatgccagcatcctgcaggagaaactgggctccctgaacctgcggtggcaggaggtgtgcaagcagctgagcgaccggaagaagcggctcgaggagcagaagaacatcctgagcgagttccagcgggacctgaacgagttcgtgctgtggctagaggaggcagacaacatagccagcatacctctggagcccggcaaagagcagcagcttaaggagaagttagagcaggtgaagcttctagtcgaggagctccctctgcggcaaggcatcctaaaacaactgaacgagacaggtggccccgtccttgtgtccgccccaattagccctgaggagcaagataaactggagaacaaacttaagcagaccaatctgcagtggatcaaggtgtcaagggccctgccagagaagcagggcgagatagaggcccaaatcaaggacctgggccagctagagaagaagcttgaggacctagaggagcaacttaaccaccttctactgtggctgagccccatccggaatcagctggagatatacaaccaacccaatcaggagggccccttcgacgtcaaagagacagagatcgccgtgcaggccaagcagccagatgtcgaggagatcctgagcaagggccagcacctctacaaagagaaaccagccacccaaccagtgaagcggaaactggaggatctgtcaagcgagtggaaggccgtgaaccggcttttacaggagctgcgggccaagcaacccgatcttgctcccggcctgacaaccataggcgcatcacccacccagacagtgaccctggtgacccagccagtggtgacaaaagagacagccataagcaaactggagatgcccagcagcctcatgctggaggtccccgctttagccgacttcaaccgggcctggaccgagttaaccgattggctgtcactgctggaccaggtgatcaaatcccagagggtgatggtcggggatctcgaggacattaacgagatgatcatcaagcagaaggccaccatgcaggaccttgagcaacgtcggccccaactcgaggagctgataaccgctgcccagaacctgaagaacaagaccagcaatcaagaggcccggaccatcatcaccgacaggatcgagcggatccaaaaccagtgggacgaggtccaggagcacctgcaaaaccgtaggcagcagctaaacgagatgctgaaggacagcacccaatggctggaggctaaagaggaggctgagcaagtcctaggccaagcccgggcaaagctggagagctggaaggagggaccctacaccgtggacgccatccagaagaaaatcacagagaccaaacagctcgccaaggacctccggcagtggcagaccaacgtggacgtggccaacgacctagctttaaaactgctgcgggactacagcgctgacgatacccggaaggtgcacatgatcactgagaacatcaatgccagctggcggagcatccacaagcgggtgtcagagcgggaggctgctctggaggagactcaccggcttcttcagcagtttcccctggacctggagaaattcctggcctggctgactgaggccgagactaccgccaacgtgttacaggacgccacccgtaaggagcggctactcgaggacagcaagggcgtgaaggagctgatgaagcagtggcaagacctgcaaggagagatcgaggcccacaccgacgtgtaccacaacctggacgagaacagccaaaagattctgaggagcctggagggaagcgacgacgctgtcttacttcagcgtcggctcgataatatgaacttcaaatggagcgagctgaggaagaagtccctgaacatccggagccaccttgaggctagcagcgaccagtggaaacggcttcacctgtccctgcaggagcttctagtgtggctgcagctgaaagacgacgagctgagcaggcaggcacccattggtggagacttccccgccgtgcagaagcagaacgatgtgcaccgggccttcaagcgggagctgaagacaaaggagcccgtgatcatgagcacactggagactgtgcggatcttcctgaccgagcagccactggagggtctggagaagctttaccaggagcctcgtgagctgccaccagaggagcgtgctcagaacgtgacccggctactgcggaagcaggccgaggaggtgaacaccgagtgggagaagctgaaccttcacagcgcagactggcagcgtaaaatcgacgagacacttgagcggctgcaagagctccaagaggccactgatgagctggatctgaagcttcgtcaagcagaggtcatcaagggctcatggcagccagtgggtgacctcctgatcgatagcctgcaggaccaccttgagaaggtgaaggcccttcggggcgagatcgcaccactaaaagagaacgtgtcccacgtgaacgatctagctcggcagctgaccaccctgggcatccagcttagcccctacaacctcagcaccctcgaggaccttaacacccggtggaagctgctgcaggtggctgtggaggaccgtgtgcggcaattacacgaggcccaccgtgactttgggcccgctagccagcacttcttatcaaccagcgtgcagggtccctgggagagggccatctcacctaacaaggtgccctactacatcaaccacgagactcagaccacctgctgggaccaccctaagatgaccgagttataccagagcctggccgacctgaataacgtgcggttctccgcctaccggacagccatgaaactgaggcggctacagaaagccctgtgcctcgacctcctgtcactcagcgctgcctgtgatgccctggaccagcacaacctgaagcagaatgaccagcccatggacatccttcaaatcatcaactgcctcaccaccatctacgaccgtcttgagcaagagcacaacaatctggtgaacgtgcccctgtgcgtggacatgtgcctgaactggctgctgaacgtgtacgacacaggccgtaccggcaggatacgggtcctgtcatttaagaccggcatcatcagcttatgcaaggcccacctcgaggacaagtaccggtacctgttcaagcaagtggccagctccactggcttctgcgaccagaggcggctaggactgctgctgcacgacagcattcagattccccggcaacttggggaggtggctagcttcggtggcagcaacatcgagccctcagtgcggagctgcttccagttcgccaacaacaagcccgagattgaggccgccttatttctggactggatgaggctggagccacagagcatggtgtggcttcccgtgttacaccgtgtggctgctgcagagaccgcaaaacaccaggccaagtgcaacatttgcaaagagtgccccatcatcggctttcggtaccggtccctgaagcacttcaattacgatatttgtcagagctgcttcttctcaggacgggtcgccaagggacacaaaatgcactaccccatggtggagtactgtacccctaccacctccggagaggacgtgcgtgatttcgctaaggtgctgaagaataagttccgtaccaagcggtacttcgccaagcacccacggatgggctacctgcccgtgcagaccgtcctggagggagacaacatggagactcccgtgacccttatcaacttttggcctgtggacagcgcacctgcctcatcccctcaactgagccacgatgacacccactcacggatcgagcactacgcttcacggctcgccgagatggagaacagcaacggcagctacctgaatgacagcattagccccaacgagagcatcgacgacgagcacctactgatccagcactactgccagagcctgaaccaggacagcccactgtcacagcctcgtagccccgcacagattttaatcagcctggagtccgaggagcgtggtgagctggagcggatcctcgccgatttagaggaggagaaccggaacctgcaggccgagtacgaccggcttaagcagcagcacgagcacaaaggactgtcaccacttcccagccctcccgagatgatgcctaccagccctcaatcacccagggatgccgagctgatcgctgaggccaagctgctgcgtcagcacaaggggcggttagaggcacggatgcaaatattagaggaccacaacaaacagttagagtcacaactgcacaggcttcggcagctgctggagcaaccacaggccgaggctaaggtgaacggcacaaccgtgagcagcccaagcacctcactccagcggagcgacagcagccagcccatgttactgcgggtggtgggctcacagaccagcgacagcatgggcgaggaggaccttctgagcccaccccaagacaccagcaccggtctggaggaggtcatggagcaactcaataacagcttcccttcctcaaggggccggaataccccaggcaagccaatgcgggaggacaccatg

SEQ ID NO: 16 - protein, 3685 aa, Homo sapiens; identical to SEQ ID NO: 2

SEQ ID NO: 17 - DNA, 4461 bp, synthetic construct
  source 1..4461: mol_type = other DNA
atgggcaagcccattcctaatcctctgctgggcctcgacagcaccatgctttggtgggaagaagtcgaggactgctacgagcgcgaggacgtgcagaagaaaaccttcaccaaatgggtcaacgcccagttcagcaagttcggcaagcagcacatcgagaacctgttcagcgacctgcaggatggcagaaggctgctggatctgctggaaggcctgacaggacagaagctgcccaaagagaagggcagcacaagagtgcacgccctgaacaacgtgaacaaggccctgagagtgctgcagaacaacaacgtggacctggtcaacatcggcagcaccgacatcgtggacggcaaccacaaactgaccctgggcctgatctggaacatcatcctgcactggcaagtgaagaacgtgatgaagaacatcatggccggcctgcagcagaccaacagcgagaagattctgctgagctgggtccgacagagcacccggaattaccctcaagtgaacgtgatcaacttcaccacctcttggagcgacggactggccctgaatgccctgatccacagccacagacctgacctgttcgactggaacagcgtcgtgtgtcagcagagcgccacacagaggctggaacacgccttcaatatcgccagataccagctgggcatcgagaaactgctggaccccgaggatgtggacaccacctatcctgacaagaaatccatcctcatgtacatcaccagcctgttccaggtgctgccccagcaggtttccatcgaggccattcaagaggtcgagatgctgcccagacctcctaaagtgaccaaagaggaacacttccagctgcaccaccagatgcactactctcagcagatcaccgtgtctctggcccagggctacgagagaacaagcagccccaagcctcggttcaagagctacgcctatacacaggccgcctacgtgaccaccagcgatcctacaagaagcccatttcctagccagcacctcgaggcccctgaggataagagctttggcagcagcctgatggaaagcgaagtgaacctggaccgctaccagacagccctggaagaggttctgagctggctgctgtctgccgaggatacactgcaggctcagggcgagatcagcaacgacgtggaagtggtcaaggaccagtttcacacccacgagggctacatgatggacctgacagcccaccagggcagagtgggcaatattctgcagctgggctccaagctgatcggcacaggcaagctgagcgaggacgaagagacagaggtgcaagagcagatgaacctgctgaacagcagatgggagtgtctgagagtggccagcatggaaaagcagagcaacctgcaccgggtgctgatggatctccagaaccagaagctgaaagagctgaacgactggctgaccaagaccgaggaacggacccggaagatggaagaggaacctctgggacccgacctggaagatctgaaaagacaggtgcagcagcataaggtgctgcaagaggacctcgagcaagagcaagtgcgcgtgaacagcctgacacacatggtggtggtcgtggatgagagcagcggagatcatgccacagccgctctggaagaacagctgaaggtgctgggagacagatgggccaatatctgccggtggaccgaggatagatgggtgctgctccaggacatcctgctgaagtggcagcggctgacagaggaacagtgcctgtttagcgcctggctgtccgagaaagaggacgccgtcaacaagatccacaccaccggcttcaaggatcagaatgagatgctgagcagcctgcagaaactggccgtgctgaaggctgacctggaaaagaaaaagcagtccatgggcaagctgtactccctgaagcaggacctgctgagcacactgaagaacaagagcgtgacccagaaaaccgaggcctggctggacaacttcgccagatgctgggacaacctggtgcagaagctggaaaagtccaccgctcagatctctcaggccgtgaccacaacacagccttctctgacccagaccaccgtgatggaaacagtgaccacagtgacaacccgcgagcagatcctggtcaagcacgcccaagaagaactgcctcctccacctcctcagaagaaacggcagatcacagtggacactctggaaaggcttcaagaattgcaagaggccaccgacgagctggacctgaagcttagacaggctgaagtcatcaaaggcagctggcagcctgtgggcgacctcctgattgatagcctgcaggaccacctcgaaaaagtgaaggccctgcggggagagatcgcccctctgaaagaaaacgtgtcccacgtgaacgacctggcacggcagcttacaacactggggattcagctgagcccctacaacctgtccacactcgaggacctgaatacccgatggaagctcctccaggtggccgttgaagatagagtgcggcagctgcacgaagcccacagagattttggaccagccagccagcactttctgagcacctcagtgcaaggcccctgggagagagctatcagccctaacaaggtgccctactacatcaaccacgagacacagacaacctgttgggatcaccccaagatgaccgaactgtatcagtccctggccgatctgaacaatgtgcggttcagcgcctaccggaccgcaatgaagctgcggagacttcagaaagccctgtgcctcgacctgctgtctctgagtgcagcatgtgatgccctggaccagcataatctgaagcagaacgaccagcctatggacatcctccagatcatcaactgcctgaccactatctacgaccggctcgaacaagaacacaacaacctcgtgaacgtgcccctgtgcgtggacatgtgcctcaattggctgctgaatgtgtacgacaccggccggaccggcagaatcagagtgctgtctttcaaaaccggcatcatctccctgtgcaaagcccatctcgaggataagtaccgctacctgtttaagcaggtcgccagcagcaccggcttttgcgatcaaagaaggctgggactcctgctgcacgacagcattcagatcccaagacagctgggcgaagtggctagcttcggcggctctaatatcgagcctagcgtgcggagctgcttccagttcgccaacaacaagcccgagattgaggcagccctcttcctggattggatgcggctggaaccccagagcatggtttggctgccagtgctgcatagagtggccgctgccgaaacagctaagcaccaggccaagtgcaacatctgcaaagagtgccccatcatcggcttccggtacagaagcctgaagcacttcaactacgatatctgccagtcttgtttcttctctggccgcgtggccaagggccacaagatgcattaccccatggtggaatactgcacccctaccacctccggcgaagatgtccgggattttgccaaggtgctcaaaaacaagttccgcaccaagcgctacttcgcaaagcaccccagaatgggctacctgcctgtgcagacagttctggaaggcgataatatggaaacccctgtgacgctgatcaatttctggcccgtggatagcgcccctgccagttctcctcagctgtctcacgatgacacccactccagaatcgagcactacgcctccagactggccgagatggaaaacagcaacggcagctatctgaacgatagcatcagccccaacgagagcatcgacgacgaacatctgctcatccagcactactgtcagagcctgaaccaggactccccactgagccagcctagaagcccagcacagattctgatttccctggaatccgaagaacggggcgagctggaaagaattctggccgacctcgaggaagaaaaccggaacctgcaggcagagtacgacagactgaaacaacagcacgagcacaagggactgagccctttgccatctcctccagaaatgatgcccacctctccacagagccccagagatgcagagctgattgccgaggccaaactcctgcgccagcacaaaggcagactcgaggccagaatgcagattctcgaagatcacaacaaacagctcgagtctcagctccaccgcctgcggcaattgctggaacaaccacaggcagaggctaaagtgaacggcaccacagtgtctagccccagcacatccctgcagagatccgatagctcccagccaatgctcctgagagtcgtgggcagccagaccagcgattctatgggcgaagaagatctgcttagccctcctcaggatacaagcaccggcctcgaagaagttatggaacaacttaacaatagcttccccagcagcagaggccgcaacacacctggcaagcccatgcgggaagataccatgtaa

SEQ ID NO: 18 - protein, 1471 aa, synthetic construct
MLWWEEVEDCYEREDVQKKTFTKWVNAQFSKFGKQHIENLFSDLQDGRRLLDLLEGLTGQKLPKEKGSTRVHALNNVNKALRVLQNNNVDLVNIGSTDIVDGNHKLTLGLIWNIILHWQVKNVMKNIMAGLQQTNSEKILLSWVRQSTRNYPQVNVINFTTSWSDGLALNALIHSHRPDLFDWNSVVCQQSATQRLEHAFNIARYQLGIEKLLDPEDVDTTYPDKKSILMYITSLFQVLPQQVSIEAIQEVEMLPRPPKVTKEEHFQLHHQMHYSQQITVSLAQGYERTSSPKPRFKSYAYTQAAYVTTSDPTRSPFPSQHLEAPEDKSFGSSLMESEVNLDRYQTALEEVLSWLLSAEDTLQAQGEISNDVEVVKDQFHTHEGYMMDLTAHQGRVGNILQLGSKLIGTGKLSEDEETEVQEQMNLLNSRWECLRVASMEKQSNLHRVLMDLQNQKLKELNDWLTKTEERTRKMEEEPLGPDLEDLKRQVQQHKVLQEDLEQEQVRVNSLTHMVVVVDESSGDHATAALEEQLKVLGDRWANICRWTEDRWVLLQDILLKWQRLTEEQCLFSAWLSEKEDAVNKIHTTGFKDQNEMLSSLQKLAVLKADLEKKKQSMGKLYSLKQDLLSTLKNKSVTQKTEAWLDNFARCWDNLVQKLEKSTAQISQAVTTTQPSLTQTTVMETVTTVTTREQILVKHAQEELPPPPPQKKRQITVDTLERLRELQEATDELDLKLRQAEVIKGSWQPVGDLLIDSLQDHLEKVKALRGEIAPLKENVSHVNDLARQLTTLGIQLSPYNLSTLEDLNTRWKLLQVAVEDRVRQLHEAHRDFGPASQHFLSTSVQGPWERAISPNKVPYYINHETQTTCWDHPKMTELYQSLADLNNVRFSAYRTAMKLRRLQKALCLDLLSLSAACDALDQHNLKQNDQPMDILQIINCLTTIYDRLEQEHNNLVNVPLCVDMCLNWLLNVYDTGRTGRIRVLSFKTGIISLCKAHLEDKYRYLFKQVASSTGFCDQRRLGLLLHDSIQIPRQLGEVASFGGSNIEPSVRSCFQFANNKPEIEAALFLDWMRLEPQSMVWLPVLHRVAAAETAKHQAKCNICKECPIIGFRYRSLKHFNYDICQSCFFSGRVAKGHKMHYPMVEYCTPTTSGEDVRDFAKVLKNKFRTKRYFAKHPRMGYLPVQTVLEGDNMETPVTLINFWPVDSAPASSPQLSHDDTHSRIEHYASRLAEMENSNGSYLNDSISPNESIDDEHLLIQHYCQSLNQDSPLSQPRSPAQILISLESEERGELERILADLEEENRNLQAEYDRLKQQHEHKGLSPLPSPPEMMPTSPQSPRDAELIAEAKLLRQHKGRLEARMQILEDHNKQLESQLHRLRQLLEQPQAEAKVNGTTVSSPSTSLQRSDSSQPMLLRVVGSQTSDSMGEEDLLSPPQDTSTGLEEVMEQLNNSFPSSRGRNTPGKPMREDTM

SEQ ID NO: 19 - DNA, 5835 bp, synthetic construct
  source 1..5835: mol_type = other DNA
atgggcaagcccattcctaatcctctgctgggcctcgacagcaccatgctttggtgggaagaagtcgaggactgctacgagcgcgaggacgtgcagaagaaaaccttcaccaaatgggtcaacgcccagttcagcaagttcggcaagcagcacatcgagaacctgttcagcgacctgcaggatggcagaaggctgctggatctgctggaaggcctgacaggacagaagctgcccaaagagaagggcagcacaagagtgcacgccctgaacaacgtgaacaaggccctgagagtgctgcagaacaacaacgtggacctggtcaacatcggcagcaccgacatcgtggacggcaaccacaaactgaccctgggcctgatctggaacatcatcctgcactggcaagtgaagaacgtgatgaagaacatcatggccggcctgcagcagaccaacagcgagaagattctgctgagctgggtccgacagagcacccggaattaccctcaagtgaacgtgatcaacttcaccacctcttggagcgacggactggccctgaatgccctgatccacagccacagacctgacctgttcgactggaacagcgtcgtgtgtcagcagagcgccacacagaggctggaacacgccttcaatatcgccagataccagctgggcatcgagaaactgctggaccccgaggatgtggacaccacctatcctgacaagaaatccatcctcatgtacatcaccagcctgttccaggtgctgccccagcaggtttccatcgaggccattcaagaggtcgagatgctgcccagacctcctaaagtgaccaaagaggaacacttccagctgcaccaccagatgcactactctcagcagatcaccgtgtctctggcccagggctacgagagaacaagcagccccaagcctcggttcaagagctacgcctatacacaggccgcctacgtgaccaccagcgatcctacaagaagcccatttcctagccagcacctcgaggcccctgaggataagagctttggcagcagcctgatggaaagcgaagtgaacctggaccgctaccagacagccctggaagaggttctgagctggctgctgtctgccgaggatacactgcaggctcagggcgagatcagcaacgacgtggaagtggtcaaggaccagtttcacacccacgagggctacatgatggacctgacagcccaccagggcagagtgggcaatattctgcagctgggctccaagctgatcggcacaggcaagctgagcgaggacgaagagacagaggtgcaagagcagatgaacctgctgaacagcagatgggagtgtctgagagtggccagcatggaaaagcagagcaacctgcaccgggtgctgatggatctccagaaccagaagctgaaagagctgaacgactggctgaccaagaccgaggaacggacccggaagatggaagaggaacctctgggacccgacctggaagatctgaaaagacaggtgcagcagcataaggtgctgcaagaggacctcgagcaagagcaagtgcgcgtgaacagcctgacacacatggtggtggtcgtggatgagagcagcggagatcatgccacagccgctctggaagaacagctgaaggtgctgggagacagatgggccaatatctgccggtggaccgaggatagatgggtgctgctccaggacatcctgctgaagtggcagcggctgacagaggaacagtgcctgtttagcgcctggctgtccgagaaagaggacgccgtcaacaagatccacaccaccggcttcaaggatcagaatgagatgctgagcagcctgcagaaactggccgtgctgaaggctgacctggaaaagaaaaagcagtccatgggcaagctgtactccctgaagcaggacctgctgagcacactgaagaacaagagcgtgacccagaaaaccgaggcctggctggacaacttcgccagatgctgggacaacctggtgcagaagctggaaaagtccaccgctcagatctctcagcagcccgatctggcccctggcctgacaacaattggagctagccccactcagaccgtgacactggttacacagccagtggtcaccaaagagacagccatctccaaactggaaatgccctctagcctgatgttggaggtgcccgcactggccgacttcaatagagcttggaccgagctgactgattggctgtccctcctggaccaagtgatcaagagccagagagtgatggtcggagatcttgaggacatcaacgaaatgattatcaagcagaaagccaccatgcaggatctcgaacagcgcagaccccagcttgaagaactgatcacagccgcacagaatctgaaaaacaagaccagcaatcaagaagccaggaccatcatcaccgaccggatcgagcggattcagaaccagtgggatgaagtccaagagcatctgcagaaccggcggcagcagcttaacgaaatgctgaaggacagcacacagtggctcgaagctaaagaagaagccgagcaggttctgggacaagccagagccaagctggaatcctggaaagagggaccctacaccgtcgacgccattcagaaaaaaatcaccgagacaaaacagctggccaaggatctgcggcagtggcagaccaatgtggacgtggcaaatgacctggctctgaaactgctccgggactacagcgccgacgacaccagaaaggtgcacatgatcacagagaacatcaacgcctcttggcggagcatccacaagcgggtgtccgaaagagaggctgccctcgaggaaacacacagactgctgcaacagtttcccctggaccttgagaaattcctggcctggctcaccgaggccgaaaccacagctaatgtcctccaggatgccacacggaaagagcggttgctggaagatagcaaaggggtcaaagaacttatgaagcaatggcaggacctccaaggggagatcgaggctcacaccgacgtgtaccacaatctggatgagaacagtcagaagatcctgcggtccctggaaggctccgatgatgctgtgttgctccagcggagactggataacatgaacttcaagtggagcgagctgcggaaaaagtccctgaacatcagaagccatctggaagccagcagcgaccagtggaaaagactccacctgagcctccaagagcttctcgtgtggctgcagctcaaggacgatgagctgagcagacaggccccaatcggcggagattttcccgccgtgcagaaacagaacgacgtgcacagagccttcaagcgggaactcaagaccaaagaacccgtgattatgagcaccctggaaactgtgcggatctttctgaccgagcagcctctggaaggactcgagaagctgtaccaagaacctagagagctgcctcctgaggaacgggcccagaacgttacacggctgctgagaaaacaggccgaggaagtgaataccgagtgggaaaagctgaatctccactccgccgactggcagcggaagatcgatgagactctggaaaggcttcaagaattgcaagaggccaccgacgagctggacctgaagcttagacaggctgaagtcatcaaaggcagctggcagcctgtgggcgacctcctgattgatagcctgcaggaccacctcgaaaaagtgaaggccctgcggggagagatcgcccctctgaaagaaaacgtgtcccacgtgaacgacctggcacggcagcttacaacactggggattcagctgagcccctacaacctgtccacactcgaggacctgaatacccgatggaagctcctccaggtggccgttgaagatagagtgcggcagctgcacgaagcccacagagattttggaccagccagccagcactttctgagcacctcagtgcaaggcccctgggagagagctatcagccctaacaaggtgccctactacatcaaccacgagacacagacaacctgttgggatcaccccaagatgaccgaactgtatcagtccctggccgatctgaacaatgtgcggttcagcgcctaccggaccgcaatgaagctgcggagacttcagaaagccctgtgcctcgacctgctgtctctgagtgcagcatgtgatgccctggaccagcataatctgaagcagaacgaccagcctatggacatcctccagatcatcaactgcctgaccactatctacgaccggctcgaacaagaacacaacaacctcgtgaacgtgcccctgtgcgtggacatgtgcctcaattggctgctgaatgtgtacgacaccggccggaccggcagaatcagagtgctgtctttcaaaaccggcatcatctccctgtgcaaagcccatctcgaggataagtaccgctacctgtttaagcaggtcgccagcagcaccggcttttgcgatcaaagaaggctgggactcctgctgcacgacagcattcagatcccaagacagctgggcgaagtggctagcttcggcggctctaatatcgagcctagcgtgcggagctgcttccagttcgccaacaacaagcccgagattgaggcagccctcttcctggattggatgcggctggaaccccagagcatggtttggctgccagtgctgcatagagtggccgctgccgaaacagctaagcaccaggccaagtgcaacatctgcaaagagtgccccatcatcggcttccggtacagaagcctgaagcacttcaactacgatatctgccagtcttgtttcttctctggccgcgtggccaagggccacaagatgcattaccccatggtggaatactgcacccctaccacctccggcgaagatgtccgggattttgccaaggtgctcaaaaacaagttccgcaccaagcgctacttcgcaaagcaccccagaatgggctacctgcctgtgcagacagttctggaaggcgataatatggaaacccctgtgacgctgatcaatttctggcccgtggatagcgcccctgccagttctcctcagctgtctcacgatgacacccactccagaatcgagcactacgcctccagactggccgagatggaaaacagcaacggcagctatctgaacgatagcatcagccccaacgagagcatcgacgacgaacatctgctcatccagcactactgtcagagcctgaaccaggactccccactgagccagcctagaagcccagcacagattctgatttccctggaatccgaagaacggggcgagctggaaagaattctggccgacctcgaggaagaaaaccggaacctgcaggcagagtacgacagactgaaacaacagcacgagcacaagggactgagccctttgccatctcctccagaaatgatgcccacctctccacagagccccagagatgcagagctgattgccgaggccaaactcctgcgccagcacaaaggcagactcgaggccagaatgcagattctcgaagatcacaacaaacagctcgagtctcagctccaccgcctgcggcaattgctggaacaaccacaggcagaggctaaagtgaacggcaccacagtgtctagccccagcacatccctgcagagatccgatagctcccagccaatgctcctgagagtcgtgggcagccagaccagcgattctatgggcgaagaagatctgcttagccctcctcaggatacaagcaccggcctcgaagaagttatggaacaacttaacaatagcttccccagcagcagaggccgcaacacacctggcaagcccatgcgggaagataccatgtaa

SEQ ID NO: 20 - protein, 1929 aa, synthetic construct
MLWWEEVEDCYEREDVQKKTFTKWVNAQFSKFGKQHIENLFSDLQDGRRLLDLLEGLTGQKLPKEKGSTRVHALNNVNKALRVLQNNNVDLVNIGSTDIVDGNHKLTLGLIWNIILHWQVKNVMKNIMAGLQQTNSEKILLSWVRQSTRNYPQVNVINFTTSWSDGLALNALIHSHRPDLFDWNSVVCQQSATQRLEHAFNIARYQLGIEKLLDPEDVDTTYPDKKSILMYITSLFQVLPQQVSIEAIQEVEMLPRPPKVTKEEHFQLHHQMHYSQQITVSLAQGYERTSSPKPRFKSYAYTQAAYVTTSDPTRSPFPSQHLEAPEDKSFGSSLMESEVNLDRYQTALEEVLSWLLSAEDTLQAQGEISNDVEVVKDQFHTHEGYMMDLTAHQGRVGNILQLGSKLIGTGKLSEDEETEVQEQMNLLNSRWECLRVASMEKQSNLHRVLMDLQNQKLKELNDWLTKTEERTRKMEEEPLGPDLEDLKRQVQQHKVLQEDLEQEQVRVNSLTHMVVVVDESSGDHATAALEEQLKVLGDRWANICRWTEDRWVLLQDILLKWQRLTEEQCLFSAWLSEKEDAVNKIHTTGFKDQNEMLSSLQKLAVLKADLEKKKQSMGKLYSLKQDLLSTLKNKSVTQKTEAWLDNFARCWDNLVQKLEKSTAQISQQPDLAPGLTTIGASPTQTVTLVTQPVVTKETAISKLEMPSSLMLEVPALADFNRAWTELTDWLSLLDQVIKSQRVMVGDLEDINEMIIKQKATMQDLEQRRPQLEELITAAQNLKNKTSNQEARTIITDRIERIQNQWDEVQEHLQNRRQQLNEMLKDSTQWLEAKEEAEQVLGQARAKLESWKEGPYTVDAIQKKITETKQLAKDLRQWQTNVDVANDLALKLLRDYSADDTRKVHMITENINASWRSIHKRVSEREAALEETHRLLQQFPLDLEKFLAWLTEAETTANVLQDATRKERLLEDSKGVKELMKQWQDLQGEIEAHTDVYHNLDENSQKILRSLEGSDDAVLLQRRLDNMNFKWSELRKKSLNIRSHLEASSDQWKRLHLSLQELLVWLQLKDDELSRQAPIGGDFPAVQKQNDVHRAFKRELKTKEPVIMSTLETVRIFLTEQPLEGLEKLYQEPRELPPEERAQNVTRLLRKQAEEVNTEWEKLNLHSADWQRKIDETLERLRELQEATDELDLKLRQAEVIKGSWQPVGDLLIDSLQDHLEKVKALRGEIAPLKENVSHVNDLARQLTTLGIQLSPYNLSTLEDLNTRWKLLQVAVEDRVRQLHEAHRDFGPASQHFLSTSVQGPWERAISPNKVPYYINHETQTTCWDHPKMTELYQSLADLNNVRFSAYRTAMKLRRLQKALCLDLLSLSAACDALDQHNLKQNDQPMDILQIINCLTTIYDRLEQEHNNLVNVPLCVDMCLNWLLNVYDTGRTGRIRVLSFKTGIISLCKAHLEDKYRYLFKQVASSTGFCDQRRLGLLLHDSIQIPRQLGEVASFGGSNIEPSVRSCFQFANNKPEIEAALFLDWMRLEPQSMVWLPVLHRVAAAETAKHQAKCNICKECPIIGFRYRSLKHFNYDICQSCFFSGRVAKGHKMHYPMVEYCTPTTSGEDVRDFAKVLKNKFRTKRYFAKHPRMGYLPVQTVLEGDNMETPVTLINFWPVDSAPASSPQLSHDDTHSRIEHYASRLAEMENSNGSYLNDSISPNESIDDEHLLIQHYCQSLNQDSPLSQPRSPAQILISLESEERGELERILADLEEENRNLQAEYDRLKQQHEHKGLSPLPSPPEMMPTSPQSPRDAELIAEAKLLRQHKGRLEARMQILEDHNKQLESQLHRLRQLLEQPQAEAKVNGTTVSSPSTSLQRSDSSQPMLLRVVGSQTSDSMGEEDLLSPPQDTSTGLEEVMEQLNNSFPSSRGRNTPGKPMREDTM

SEQ ID NO: 21 - DNA, 6000 bp, synthetic construct
  source 1..6000: mol_type = other DNA
atgctttggtgggaagaagtcgaggactgctacgagcgcgaggacgtgcagaagaaaaccttcaccaaatgggtcaacgcccagttcagcaagttcggcaagcagcacatcgagaacctgttcagcgacctgcaggatggcagaaggctgctggatctgctggaaggcctgacaggacagaagctgcccaaagagaagggcagcacaagagtgcacgccctgaacaacgtgaacaaggccctgagagtgctgcagaacaacaacgtggacctggtcaacatcggcagcaccgacatcgtggacggcaaccacaaactgaccctgggcctgatctggaacatcatcctgcactggcaagtgaagaacgtgatgaagaacatcatggccggcctgcagcagaccaacagcgagaagattctgctgagctgggtccgacagagcacccggaattaccctcaagtgaacgtgatcaacttcaccacctcttggagcgacggactggccctgaatgccctgatccacagccacagacctgacctgttcgactggaacagcgtcgtgtgtcagcagagcgccacacagaggctggaacacgccttcaatatcgccagataccagctgggcatcgagaaactgctggaccccgaggatgtggacaccacctatcctgacaagaaatccatcctcatgtacatcaccagcctgttccaggtgctgccccagcaggtttccatcgaggccattcaagaggtcgagatgctgcccagacctcctaaagtgaccaaagaggaacacttccagctgcaccaccagatgcactactctcagcagatcaccgtgtctctggcccagggctacgagagaacaagcagccccaagcctcggttcaagagctacgcctatacacaggccgcctacgtgaccaccagcgatcctacaagaagcccatttcctagccagcacctcgaggcccctgaggataagagctttggcagcagcctgatggaaagcgaagtgaacctggaccgctaccagacagccctggaagaggttctgagctggctgctgtctgccgaggatacactgcaggctcagggcgagatcagcaacgacgtggaagtggtcaaggaccagtttcacacccacgagggctacatgatggacctgacagcccaccagggcagagtgggcaatattctgcagctgggctccaagctgatcggcacaggcaagctgagcgaggacgaagagacagaggtgcaagagcagatgaacctgctgaacagcagatgggagtgtctgagagtggccagcatggaaaagcagagcaacctgcaccgggtgctgatggatctccagaaccagaagctgaaagagctgaacgactggctgaccaagaccgaggaacggacccggaagatggaagaggaacctctgggacccgacctggaagatctgaaaagacaggtgcagcagcataaggtgctgcaagaggacctcgagcaagagcaagtgcgcgtgaacagcctgacacacatggtggtggtcgtggatgagagcagcggagatcatgccacagccgctctggaagaacagctgaaggtgctgggagacagatgggccaatatctgccggtggaccgaggatagatgggtgctgctccaggacatcctgctgaagtggcagcggctgacagaggaacagtgcctgtttagcgcctggctgtccgagaaagaggacgccgtcaacaagatccacaccaccggcttcaaggatcagaatgagatgctgagcagcctgcagaaactggccgtgctgaaggctgacctggaaaagaaaaagcagtccatgggcaagctgtactccctgaagcaggacctgctgagcacactgaagaacaagagcgtgacccagaaaaccgaggcctggctggacaacttcgccagatgctgggacaacctggtgcagaagctggaaaagtccaccgctcagatcagccagatctacaaccagcctaatcaagagggccccttcgacgtgaaagaaaccgagattgccgtgcaggccaagcagccagatgtggaagagatcctgagcaagggacagcacctgtacaaagagaaacccgccactcagcccgtgaagagaaagctcgaggatctgagcagcgagtggaaggccgtgaacagactcctgcaagagctgagagccaaacagcccgatctggcccctggcctgacaacaattggagctagccccacacagaccgtgacactggttacacagcccgtggtcaccaaagagacagccatcagcaaactggaaatgccctctagcctgatgctcgaagtgcccgctctggccgacttcaatagagcctggaccgagctgacagattggctgagcctcctggaccaagtgatcaagagccagagagtgatggtcggagatctcgaggacatcaacgagatgatcattaagcagaaagccaccatgcaggatctcgaacagagaaggccccagctcgaagaactgatcaccgccgcacagaacctgaaaaacaagaccagcaatcaagaagccaggaccatcatcaccgaccggatcgagcggatccagaatcagtgggacgaagtgcaagaacatctgcagaaccggcggcagcagctcaacgaaatgctgaaggatagcacccagtggctggaagccaaagaagaggccgagcaggttctgggacaagccagagccaagctggaatcctggaaagagggaccctacaccgtcgacgccatccagaagaagatcaccgagacaaagcagctggccaaggacctgagacagtggcagaccaatgtggacgtggccaacgatctggctctgaagctgctgagggactacagcgccgacgacaccagaaaggtgcacatgatcacagagaacatcaacgcctcttggcggagcatccacaagcgggtgtccgaaagagaggccgctctcgaggaaacccacagactgcttcagcagttccctctggatctcgagaagttcctggcttggctgaccgaggccgaaaccacagctaacgttctgcaggacgccacacggaaagagagactgctcgaggactccaagggcgtcaaagaactcatgaagcagtggcaggatctgcagggcgaaatcgaggcccacaccgacgtgtaccacaacctggacgagaactcccagaagatcctgcggagcctggaaggcagtgatgatgctgtgctcctccagcggagactggacaacatgaacttcaagtggagcgagctgcgcaagaagtccctgaacatcagaagccacctggaagcctccagcgaccagtggaaaagactgcatctgtccctgcaagaactgctcgtgtggctgcagctcaaggacgacgaactgagcagacaggcccctatcggcggagattttcccgccgtgcagaaacagaacgacgtgcacagagccttcaagcgcgagctgaaaacaaaagaacccgtcatcatgagcaccctggaaaccgtgcggatcttcctgacagagcagcctctcgaaggactggaaaagctgtaccaagagcctcgcgagctgcctcctgaagaaagggcccagaatgtgacccggctgctgagaaagcaggccgaggaagtgaataccgagtgggagaaactgaacctgcactccgccgactggcagcggaagatcgatgagacactggaacggctgcgggaactgcaagaagctaccgacgagctggacctgaagctgagacaggccgaagtcatcaaaggatcctggcagcctgtgggcgacctgctcatcgattctctgcaggatcacctcgagaaagtgaaggccctgcggggagaaatcgcccctctgaaagaaaacgtgtcccacgtgaacgacctggccagacagctgacaacactgggcatccagctgagcccctacaacctgtccacactcgaggacctgaacacccggtggaaactgctccaagtggccgtggaagatagagtgcggcagctgcacgaagcccacagagattttggaccagccagccagcacttcctgagcacctcagttcaaggcccctgggagagagctatcagccctaacaaggtgccctactacatcaaccacgagacacagaccacctgttgggatcaccccaagatgaccgaactgtatcagtccctggccgatctgaacaatgtgcggttcagcgcctaccggaccgctatgaagctgcgaaggctgcagaaagctctgtgcctggacctgctctctctgagcgctgcttgtgatgccctggaccagcataatctgaagcagaacgaccagcctatggacatcctccagatcatcaattgcctgaccaccatctacgaccgcctggaacaagaacacaacaacctcgtgaacgtgcccctgtgcgtggacatgtgcctgaattggctgctgaacgtgtacgacaccggccggacaggcagaatcagagtgctgtccttcaagaccggcatcatctccctgtgcaaggcccaccttgaggacaagtaccgctacctgttcaaacaggtggcctccagcaccggcttttgcgaccaaagaaggctgggactgctcctgcacgacagcattcagatccctagacagctgggcgaagtggcctctttcggcggctctaatatcgagcctagcgtgcggagctgcttccagttcgccaacaacaagcccgagatcgaggctgccctgttcctggactggatgagactggaaccccagagcatggtctggctgcctgtgctgcatagagtggccgctgccgaaacagccaagcaccaggccaagtgcaacatctgtaaagagtgccccatcatcggcttccggtacagaagcctgaagcacttcaactacgatatctgccagagctgtttcttcagcggcagagtcgccaagggccacaaaatgcactaccccatggtggaatactgcacccctaccacctccggcgaagatgtgcgggattttgccaaggtgctcaaaaacaagttccggaccaagcgctacttcgctaagcaccccagaatgggctatctgcccgtgcagacagtgctcgagggcgacaatatggaaacccctgtgacactgatcaatttctggcccgtggacagcgcccctgctagttctcctcaactgagccacgacgatacccacagcaggatcgagcactacgccagcagactggccgagatggaaaacagcaacggcagctacctgaacgatagcatcagccccaacgagagcatcgacgacgagcatctgctgatccagcactactgtcagagcctgaaccaggactccccactgagccagcctagaagccctgcacagatcctgatcagcctggaatccgaggaaaggggcgagctggaaagaatcctggccgacctcgaagaggaaaaccggaacctgcaggcagagtacgacagactgaagcagcagcacgagcacaagggactgtctccactgccatctccacctgaaatgatgcccacctctccacagagccccagagatgccgagctgattgccgaagccaaactcctcagacagcacaaaggcagactggaagcccggatgcagattcttgaggaccacaacaaacagctcgagtctcagctgcacagactgagacagctgcttgagcaacctcaggctgaggccaaagtgaacggcaccacagtgtctagcccctccactagtctgcagcggagcgattctagccagccaatgctcctgagagtcgtgggctctcagaccagcgatagcatgggcgaagaggatctgctcagccctcctcaggatacaagcaccggcctggaagaagttatggaacagctcaacaacagcttccccagcagcagaggcagaaacacccctggcaagcccatgcgcgaagataccatg

SEQ ID NO: 22 - protein, 2000 aa, synthetic construct
MLWWEEVEDCYEREDVQKKTFTKWVNAQFSKFGKQHIENLFSDLQDGRRLLDLLEGLTGQKLPKEKGSTRVHALNNVNKALRVLQNNNVDLVNIGSTDIVDGNHKLTLGLIWNIILHWQVKNVMKNIMAGLQQTNSEKILLSWVRQSTRNYPQVNVINFTTSWSDGLALNALIHSHRPDLFDWNSVVCQQSATQRLEHAFNIARYQLGIEKLLDPEDVDTTYPDKKSILMYITSLFQVLPQQVSIEAIQEVEMLPRPPKVTKEEHFQLHHQMHYSQQITVSLAQGYERTSSPKPRFKSYAYTQAAYVTTSDPTRSPFPSQHLEAPEDKSFGSSLMESEVNLDRYQTALEEVLSWLLSAEDTLQAQGEISNDVEVVKDQFHTHEGYMMDLTAHQGRVGNILQLGSKLIGTGKLSEDEETEVQEQMNLLNSRWECLRVASMEKQSNLHRVLMDLQNQKLKELNDWLTKTEERTRKMEEEPLGPDLEDLKRQVQQHKVLQEDLEQEQVRVNSLTHMVVVVDESSGDHATAALEEQLKVLGDRWANICRWTEDRWVLLQDILLKWQRLTEEQCLFSAWLSEKEDAVNKIHTTGFKDQNEMLSSLQKLAVLKADLEKKKQSMGKLYSLKQDLLSTLKNKSVTQKTEAWLDNFARCWDNLVQKLEKSTAQISQIYNQPNQEGPFDVKETEIAVQAKQPDVEEILSKGQHLYKEKPATQPVKRKLEDLSSEWKAVNRLLQELRAKQPDLAPGLTTIGASPTQTVTLVTQPVVTKETAISKLEMPSSLMLEVPALADFNRAWTELTDWLSLLDQVIKSQRVMVGDLEDINEMIIKQKATMQDLEQRRPQLEELITAAQNLKNKTSNQEARTIITDRIERIQNQWDEVQEHLQNRRQQLNEMLKDSTQWLEAKEEAEQVLGQARAKLESWKEGPYTVDAIQKKITETKQLAKDLRQWQTNVDVANDLALKLLRDYSADDTRKVHMITENINASWRSIHKRVSEREAALEETHRLLQQFPLDLEKFLAWLTEAETTANVLQDATRKERLLEDSKGVKELMKQWQDLQGEIEAHTDVYHNLDENSQKILRSLEGSDDAVLLQRRLDNMNFKWSELRKKSLNIRSHLEASSDQWKRLHLSLQELLVWLQLKDDELSRQAPIGGDFPAVQKQNDVHRAFKRELKTKEPVIMSTLETVRIFLTEQPLEGLEKLYQEPRELPPEERAQNVTRLLRKQAEEVNTEWEKLNLHSADWQRKIDETLERLRELQEATDELDLKLRQAEVIKGSWQPVGDLLIDSLQDHLEKVKALRGEIAPLKENVSHVNDLARQLTTLGIQLSPYNLSTLEDLNTRWKLLQVAVEDRVRQLHEAHRDFGPASQHFLSTSVQGPWERAISPNKVPYYINHETQTTCWDHPKMTELYQSLADLNNVRFSAYRTAMKLRRLQKALCLDLLSLSAACDALDQHNLKQNDQPMDILQIINCLTTIYDRLEQEHNNLVNVPLCVDMCLNWLLNVYDTGRTGRIRVLSFKTGIISLCKAHLEDKYRYLFKQVASSTGFCDQRRLGLLLHDSIQIPRQLGEVASFGGSNIEPSVRSCFQFANNKPEIEAALFLDWMRLEPQSMVWLPVLHRVAAAETAKHQAKCNICKECPIIGFRYRSLKHFNYDICQSCFFSGRVAKGHKMHYPMVEYCTPTTSGEDVRDFAKVLKNKFRTKRYFAKHPRMGYLPVQTVLEGDNMETPVTLINFWPVDSAPASSPQLSHDDTHSRIEHYASRLAEMENSNGSYLNDSISPNESIDDEHLLIQHYCQSLNQDSPLSQPRSPAQILISLESEERGELERILADLEEENRNLQAEYDRLKQQHEHKGLSPLPSPPEMMPTSPQSPRDAELIAEAKLLRQHKGRLEARMQILEDHNKQLESQLHRLRQLLEQPQAEAKVNGTTVSSPSTSLQRSDSSQPMLLRVVGSQTSDSMGEEDLLSPPQDTSTGLEEVMEQLNNSFPSSRGRNTPGKPMREDTM

SEQ ID NO: 23 - DNA, 3864 bp, synthetic construct
  source 1..3864: mol_type = other DNA
atgctttggtgggaagaagtagaggactgttatgaaagagaagatgttcaaaagaaaacattcacaaaatgggtaaatgcacaattttctaagtttgggaagcagcatattgagaacctcttcagtgacctacaggatgggaggcgcctcctagacctcctcgaaggcctgacagggcaaaaactgccaaaagaaaaaggatccacaagagttcatgccctgaacaatgtcaacaaggcactgcgggttttgcagaacaataatgttgatttagtgaatattggaagtactgacatcgtagatggaaatcataaactgactcttggtttgatttggaatataatcctccactggcaggtcaaaaatgtaatgaaaaatatcatggctggattgcaacaaaccaacagtgaaaagattctcctgagctgggtccgacaatcaactcgtaattatccacaggttaatgtaatcaacttcaccaccagctggtctgatggcctggctttgaatgctctcatccatagtcataggccagacctatttgactggaatagtgtggtttgccagcagtcagccacacaacgactggaacatgcattcaacatcgccagatatcaattaggcatagagaaactactcgatcctgaagatgttgataccacctatccagataagaagtccatcttaatgtacatcacatcactcttccaagttttgcctcaacaagtgagcattgaagccatccaggaagtggaaatgttgccaaggccacctaaagtgactaaagaagaacattttcagttacatcatcaaatgcactattctcaacagatcacggtcagtctagcacagggatatgagagaacttcttcccctaagcctcgattcaagagctatgcctacacacaggctgcttatgtcaccacctctgaccctacacggagcccatttccttcacagcatttggaagctcctgaagacaagtcatttggcagttcattgatggagagtgaagtaaacctggaccgttatcaaacagctttagaagaagtattatcgtggcttctttctgctgaggacacattgcaagcacaaggagagatttctaatgatgtggaagtggtgaaagaccagtttcatactcatgaggggtacatgatggatttgacagcccatcagggccgggttggtaatattctacaattgggaagtaagctgattggaacaggaaaattatcagaagatgaagaaactgaagtacaagagcagatgaatctcctaaattcaagatgggaatgcctcagggtagctagcatggaaaaacaaagcaatttacatagaattcacactgtccgtgaagaaacgatgatggtgatgactgaagacatgcctttggaaatttcttatgtgccttctacttatttgactgaaatcactcatgtctcacaagccctattagaagtggaacaacttctcaatgctcctgacctctgtgctaaggactttgaagatctctttaagcaagaggagtctctgaagaatataaaagatagtctacaacaaagctcaggtcggattgacattattcatagcaagaagacagcagcattgcaaagtgcaacgcctgtggaaagggtgaagctacaggaagctctctcccagcttgatttccaatgggaaaaagttaacaaaatgtacaaggaccgacaagggcgatttgacagatctgttgagaaatggcggcgttttcattatgatataaagatatttaatcagtggctaacagaagctgaacagtttctcagaaagacacaaattcctgagaattgggaacatgctaaatacaaatggtatcttaaggaactccaggatggcattgggcagcggcaaactgttgtcagaacattgaatgcaactggggaagaaataattcagcaatcctcaaaaacagatgccagtattctacaggaaaaattgggaagcctgaatctgcggtggcaggaggtctgcaaacagctgtcagacagaaaaaagaggctagaagaaagttctgaccagtggaagcgtctgcacctttctctgcaggaacttctggtgtggctacagctgaaagatgatgaattaagccggcaggcacctattggaggcgactttccagcagttcagaagcagaacgatgtacatagggccttcaagagggaattgaaaactaaagaacctgtaatcatgagtactcttgagactgtacgaatatttctgacagagcagcctttggaaggactagagaaactctaccaggagcccagagagctgcctcctgaggagagagcccagaatgtcactcggcttctacgaaagcaggctgaggaggtcaatactgagtgggaaaaattgaacctgcactccgctgactggcagagaaaaatagatgagacccttgaaagactccaggaacttcaagaggccacggatgagctggacctcaagctgcgccaagctgaggtgatcaagggatcctggcagcccgtgggcgatctcctcattgactctctccaagatcacctcgagaaagtcaaggcacttcgaggagaaattgcgcctctgaaagagaacgtgagccacgtcaatgaccttgctcgccagcttaccactttgggcattcagctctcaccgtataacctcagcactctggaagacctgaacaccagatggaagcttctgcaggtggccgtcgaggaccgagtcaggcagctgcatgaagcccacagggactttggtccagcatctcagcactttctttccacgtctgtccagggtccctgggagagagccatctcgccaaacaaagtgccctactatatcaaccacgagactcaaacaacttgctgggaccatcccaaaatgacagagctctaccagtctttagctgacctgaataatgtcagattctcagcttataggactgccatgaaactccgaagactgcagaaggccctttgcttggatctcttgagcctgtcagctgcatgtgatgccttggaccagcacaacctcaagcaaaatgaccagcccatggatatcctgcagattattaattgtttgaccactatttatgaccgcctggagcaagagcacaacaatttggtcaacgtccctctctgcgtggatatgtgtctgaactggctgctgaatgtttatgatacgggacgaacagggaggatccgtgtcctgtcttttaaaactggcatcatttccctgtgtaaagcacatttggaagacaagtacagataccttttcaagcaagtggcaagttcaacaggattttgtgaccagcgcaggctgggcctccttctgcatgattctatccaaattccaagacagttgggtgaagttgcatcctttgggggcagtaacattgagccaagtgtccggagctgcttccaatttgctaataataagccagagatcgaagcggccctcttcctagactggatgagactggaaccccagtccatggtgtggctgcccgtcctgcacagagtggctgctgcagaaactgccaagcatcaggccaaatgtaacatctgcaaagagtgtccaatcattggattcaggtacaggagtctaaagcactttaattatgacatctgccaaagctgctttttttctggtcgagttgcaaaaggccataaaatgcactatcccatggtggaatattgcactccgactacatcaggagaagatgttcgagactttgccaaggtactaaaaaacaaatttcgaaccaaaaggtattttgcgaagcatccccgaatgggctacctgccagtgcagactgtcttagagggggacaacatggaaact

SEQ ID NO: 24 - protein, 1288 aa, synthetic construct
MLWWEEVEDCYEREDVQKKTFTKWVNAQFSKFGKQHIENLFSDLQDGRRLLDLLEGLTGQKLPKEKGSTRVHALNNVNKALRVLQNNNVDLVNIGSTDIVDGNHKLTLGLIWNIILHWQVKNVMKNIMAGLQQTNSEKILLSWVRQSTRNYPQVNVINFTTSWSDGLALNALIHSHRPDLFDWNSVVCQQSATQRLEHAFNIARYQLGIEKLLDPEDVDTTYPDKKSILMYITSLFQVLPQQVSIEAIQEVEMLPRPPKVTKEEHFQLHHQMHYSQQITVSLAQGYERTSSPKPRFKSYAYTQAAYVTTSDPTRSPFPSQHLEAPEDKSFGSSLMESEVNLDRYQTALEEVLSWLLSAEDTLQAQGEISNDVEVVKDQFHTHEGYMMDLTAHQGRVGNILQLGSKLIGTGKLSEDEETEVQEQMNLLNSRWECLRVASMEKQSNLHRIHTVREETMMVMTEDMPLEISYVPSTYLTEITHVSQALLEVEQLLNAPDLCAKDFEDLFKQEESLKNIKDSLQQSSGRIDIIHSKKTAALQSATPVERVKLQEALSQLDFQWEKVNKMYKDRQGRFDRSVEKWRRFHYDIKIFNQWLTEAEQFLRKTQIPENWEHAKYKWYLKELQDGIGQRQTVVRTLNATGEEIIQQSSKTDASILQEKLGSLNLRWQEVCKQLSDRKKRLEESSDQWKRLHLSLQELLVWLQLKDDELSRQAPIGGDFPAVQKQNDVHRAFKRELKTKEPVIMSTLETVRIFLTEQPLEGLEKLYQEPRELPPEERAQNVTRLLRKQAEEVNTEWEKLNLHSADWQRKIDETLERLQELQEATDELDLKLRQAEVIKGSWQPVGDLLIDSLQDHLEKVKALRGEIAPLKENVSHVNDLARQLTTLGIQLSPYNLSTLEDLNTRWKLLQVAVEDRVRQLHEAHRDFGPASQHFLSTSVQGPWERAISPNKVPYYINHETQTTCWDHPKMTELYQSLADLNNVRFSAYRTAMKLRRLQKALCLDLLSLSAACDALDQHNLKQNDQPMDILQIINCLTTIYDRLEQEHNNLVNVPLCVDMCLNWLLNVYDTGRTGRIRVLSFKTGIISLCKAHLEDKYRYLFKQVASSTGFCDQRRLGLLLHDSIQIPRQLGEVASFGGSNIEPSVRSCFQFANNKPEIEAALFLDWMRLEPQSMVWLPVLHRVAAAETAKHQAKCNICKECPIIGFRYRSLKHFNYDICQSCFFSGRVAKGHKMHYPMVEYCTPTTSGEDVRDFAKVLKNKFRTKRYFAKHPRMGYLPVQTVLEGDNMET

SEQ ID NO: 25 - DNA, 4278 bp, synthetic construct
  source 1..4278: mol_type = other DNA
atgctttggtgggaagaagtagaggactgttatgaaagagaagatgttcaaaagaaaacattcacaaaatgggtaaatgcacaattttctaagtttgggaagcagcatattgagaacctcttcagtgacctacaggatgggaggcgcctcctagacctcctcgaaggcctgacagggcaaaaactgccaaaagaaaaaggatccacaagagttcatgccctgaacaatgtcaacaaggcactgcgggttttgcagaacaataatgttgatttagtgaatattggaagtactgacatcgtagatggaaatcataaactgactcttggtttgatttggaatataatcctccactggcaggtcaaaaatgtaatgaaaaatatcatggctggattgcaacaaaccaacagtgaaaagattctcctgagctgggtccgacaatcaactcgtaattatccacaggttaatgtaatcaacttcaccaccagctggtctgatggcctggctttgaatgctctcatccatagtcataggccagacctatttgactggaatagtgtggtttgccagcagtcagccacacaacgactggaacatgcattcaacatcgccagatatcaattaggcatagagaaactactcgatcctgaagatgttgataccacctatccagataagaagtccatcttaatgtacatcacatcactcttccaagttttgcctcaacaagtgagcattgaagccatccaggaagtggaaatgttgccaaggccacctaaagtgactaaagaagaacattttcagttacatcatcaaatgcactattctcaacagatcacggtcagtctagcacagggatatgagagaacttcttcccctaagcctcgattcaagagctatgcctacacacaggctgcttatgtcaccacctctgaccctacacggagcccatttccttcacagcatttggaagctcctgaagacaagtcatttggcagttcattgatggagagtgaagtaaacctggaccgttatcaaacagctttagaagaagtattatcgtggcttctttctgctgaggacacattgcaagcacaaggagagatttctaatgatgtggaagtggtgaaagaccagtttcatactcatgaggggtacatgatggatttgacagcccatcagggccgggttggtaatattctacaattgggaagtaagctgattggaacaggaaaattatcagaagatgaagaaactgaagtacaagagcagatgaatctcctaaattcaagatgggaatgcctcagggtagctagcatggaaaaacaaagcaatttacatagagttttaatggatctccagaatcagaaactgaaagagttgaatgactggctaacaaaaacagaagaaagaacaaggaaaatggaggaagagcctcttggacctgatcttgaagacctaaaacgccaagtacaacaacataaggtgcttcaagaagatctagaacaagaacaagtcagggtcaattctctcactcacatggtggtggtagttgatgaatctagtggagatcacgcaactgctgctttggaagaacaacttaaggtattgggagatcgatgggcaaacatctgtagatggacagaagaccgctgggttcttttacaagacatccttctcaaatggcaacgtcttactgaagaacagtgcctttttagtgcatggctttcagaaaaagaagatgcagtgaacaagattcacacaactggctttaaagatcaaaatgaaatgttatcaagtcttcaaaaactggccgttttaaaagcggatctagaaaagaaaaagcaatccatgggcaaactgtattcactcaaacaagatcttctttcaacactgaagaataagtcagtgacccagaagacggaagcatggctggataactttgcccggtgttgggataatttagtccaaaaacttgaaaagagtacagcacagatttcacagcagcctgacctagctcctggactgaccactattggagcctctcctactcagactgttactctggtgacacaacctgtggttactaaggaaactgccatctccaaactagaaatgccatcttccttgatgttggaggtacctattcacactgtccgtgaagaaacgatgatggtgatgactgaagacatgcctttggaaatttcttatgtgccttctacttatttgactgaaatcactcatgtctcacaagccctattagaagtggaacaacttctcaatgctcctgacctctgtgctaaggactttgaagatctctttaagcaagaggagtctctgaagaatataaaagatagtctacaacaaagctcaggtcggattgacattattcatagcaagaagacagcagcattgcaaagtgcaacgcctgtggaaagggtgaagctacaggaagctctctcccagcttgatttccaatgggaaaaagttaacaaaatgtacaaggaccgacaagggcgatttgacagatctgttgagaaatggcggcgttttcattatgatataaagatatttaatcagtggctaacagaagctgaacagtttctcagaaagacacaaattcctgagaattgggaacatgctaaatacaaatggtatcttaaggaactccaggatggcattgggcagcggcaaactgttgtcagaacattgaatgcaactggggaagaaataattcagcaatcctcaaaaacagatgccagtattctacaggaaaaattgggaagcctgaatctgcggtggcaggaggtctgcaaacagctgtcagacagaaaaaagaggctagaagaaacccttgaaagactccaggaacttcaagaggccacggatgagctggacctcaagctgcgccaagctgaggtgatcaagggatcctggcagcccgtgggcgatctcctcattgactctctccaagatcacctcgagaaagtcaaggcacttcgaggagaaattgcgcctctgaaagagaacgtgagccacgtcaatgaccttgctcgccagcttaccactttgggcattcagctctcaccgtataacctcagcactctggaagacctgaacaccagatggaagcttctgcaggtggccgtcgaggaccgagtcaggcagctgcatgaagcccacagggactttggtccagcatctcagcactttctttccacgtctgtccagggtccctgggagagagccatctcgccaaacaaagtgccctactatatcaaccacgagactcaaacaacttgctgggaccatcccaaaatgacagagctctaccagtctttagctgacctgaataatgtcagattctcagcttataggactgccatgaaactccgaagactgcagaaggccctttgcttggatctcttgagcctgtcagctgcatgtgatgccttggaccagcacaacctcaagcaaaatgaccagcccatggatatcctgcagattattaattgtttgaccactatttatgaccgcctggagcaagagcacaacaatttggtcaacgtccctctctgcgtggatatgtgtctgaactggctgctgaatgtttatgatacgggacgaacagggaggatccgtgtcctgtcttttaaaactggcatcatttccctgtgtaaagcacatttggaagacaagtacagataccttttcaagcaagtggcaagttcaacaggattttgtgaccagcgcaggctgggcctccttctgcatgattctatccaaattccaagacagttgggtgaagttgcatcctttgggggcagtaacattgagccaagtgtccggagctgcttccaatttgctaataataagccagagatcgaagcggccctcttcctagactggatgagactggaaccccagtccatggtgtggctgcccgtcctgcacagagtggctgctgcagaaactgccaagcatcaggccaaatgtaacatctgcaaagagtgtccaatcattggattcaggtacaggagtctaaagcactttaattatgacatctgccaaagctgctttttttctggtcgagttgcaaaaggccataaaatgcactatcccatggtggaatattgcactccgactacatcaggagaagatgttcgagactttgccaaggtactaaaaaacaaatttcgaaccaaaaggtattttgcgaagcatccccgaatgggctacctgccagtgcagactgtcttagagggggacaacatggaaact

SEQ ID NO: 26 - protein, 1426 aa, synthetic construct
MLWWEEVEDCYEREDVQKKTFTKWVNAQFSKFGKQHIENLFSDLQDGRRLLDLLEGLTGQKLPKEKGSTRVHALNNVNKALRVLQNNNVDLVNIGSTDIVDGNHKLTLGLIWNIILHWQVKNVMKNIMAGLQQTNSEKILLSWVRQSTRNYPQVNVINFTTSWSDGLALNALIHSHRPDLFDWNSVVCQQSATQRLEHAFNIARYQLGIEKLLDPEDVDTTYPDKKSILMYITSLFQVLPQQVSIEAIQEVEMLPRPPKVTKEEHFQLHHQMHYSQQITVSLAQGYERTSSPKPRFKSYAYTQAAYVTTSDPTRSPFPSQHLEAPEDKSFGSSLMESEVNLDRYQTALEEVLSWLLSAEDTLQAQGEISNDVEVVKDQFHTHEGYMMDLTAHQGRVGNILQLGSKLIGTGKLSEDEETEVQEQMNLLNSRWECLRVASMEKQSNLHRVLMDLQNQKLKELNDWLTKTEERTRKMEEEPLGPDLEDLKRQVQQHKVLQEDLEQEQVRVNSLTHMVVVVDESSGDHATAALEEQLKVLGDRWANICRWTEDRWVLLQDILLKWQRLTEEQCLFSAWLSEKEDAVNKIHTTGFKDQNEMLSSLQKLAVLKADLEKKKQSMGKLYSLKQDLLSTLKNKSVTQKTEAWLDNFARCWDNLVQKLEKSTAQISQQPDLAPGLTTIGASPTQTVTLVTQPVVTKETAISKLEMPSSLMLEVPIHTVREETMMVMTEDMPLEISYVPSTYLTEITHVSQALLEVEQLLNAPDLCAKDFEDLFKQEESLKNIKDSLQQSSGRIDIIHSKKTAALQSATPVERVKLQEALSQLDFQWEKVNKMYKDRQGRFDRSVEKWRRFHYDIKIFNQWLTEAEQFLRKTQIPENWEHAKYKWYLKELQDGIGQRQTVVRTLNATGEEIIQQSSKTDASILQEKLGSLNLRWQEVCKQLSDRKKRLEETLERLQELQEATDELDLKLRQAEVIKGSWQPVGDLLIDSLQDHLEKVKALRGEIAPLKENVSHVNDLARQLTTLGIQLSPYNLSTLEDLNTRWKLLQVAVEDRVRQLHEAHRDFGPASQHFLSTSVQGPWERAISPNKVPYYINHETQTTCWDHPKMTELYQSLADLNNVRFSAYRTAMKLRRLQKALCLDLLSLSAACDALDQHNLKQNDQPMDILQIINCLTTIYDRLEQEHNNLVNVPLCVDMCLNWLLNVYDTGRTGRIRVLSFKTGIISLCKAHLEDKYRYLFKQVASSTGFCDQRRLGLLLHDSIQIPRQLGEVASFGGSNIEPSVRSCFQFANNKPEIEAALFLDWMRLEPQSMVWLPVLHRVAAAETAKHQAKCNICKECPIIGFRYRSLKHFNYDICQSCFFSGRVAKGHKMHYPMVEYCTPTTSGEDVRDFAKVLKNKFRTKRYFAKHPRMGYLPVQTVLEGDNMET

SEQ ID NO: 27 - DNA, 5463 bp, synthetic construct
  source 1..5463: mol_type = other DNA
atgctttggtgggaagaagtcgaggactgctacgagcgcgaggacgtgcagaagaaaaccttcaccaaatgggtcaacgcccagttcagcaagttcggcaagcagcacatcgagaacctgttcagcgacctgcaggatggcagaaggctgctggatctgctggaaggcctgacaggacagaagctgcccaaagagaagggcagcacaagagtgcacgccctgaacaacgtgaacaaggccctgagagtgctgcagaacaacaacgtggacctggtcaacatcggcagcaccgacatcgtggacggcaaccacaaactgaccctgggcctgatctggaacatcatcctgcactggcaagtgaagaacgtgatgaagaacatcatggccggcctgcagcagaccaacagcgagaagattctgctgagctgggtccgacagagcacccggaattaccctcaagtgaacgtgatcaacttcaccacctcttggagcgacggactggccctgaatgccctgatccacagccacagacctgacctgttcgactggaacagcgtcgtgtgtcagcagagcgccacacagaggctggaacacgccttcaatatcgccagataccagctgggcatcgagaaactgctggaccccgaggatgtggacaccacctatcctgacaagaaatccatcctcatgtacatcaccagcctgttccaggtgctgccccagcaggtttccatcgaggccattcaagaggtcgagatgctgcccagacctcctaaagtgaccaaagaggaacacttccagctgcaccaccagatgcactactctcagcagatcaccgtgtctctggcccagggctacgagagaacaagcagccccaagcctcggttcaagagctacgcctatacacaggccgcctacgtgaccaccagcgatcctacaagaagcccatttcctagccagcacctcgaggcccctgaggataagagctttggcagcagcctgatggaaagcgaagtgaacctggaccgctaccagacagccctggaagaggttctgagctggctgctgtctgccgaggatacactgcaggctcagggcgagatcagcaacgacgtggaagtggtcaaggaccagtttcacacccacgagggctacatgatggacctgacagcccaccagggcagagtgggcaatattctgcagctgggctccaagctgatcggcacaggcaagctgagcgaggacgaagagacagaggtgcaagagcagatgaacctgctgaacagcagatgggagtgtctgagagtggccagcatggaaaagcagagcaacctgcaccgggtgctgatggatctccagaaccagaagctgaaagagctgaacgactggctgaccaagaccgaggaacggacccggaagatggaagaggaacctctgggacccgacctggaagatctgaaaagacaggtgcagcagcataaggtgctgcaagaggacctcgagcaagagcaagtgcgcgtgaacagcctgacacacatggtggtggtcgtggatgagagcagcggagatcatgccacagccgctctggaagaacagctgaaggtgctgggagacagatgggccaatatctgccggtggaccgaggatagatgggtgctgctccaggacatcctgctgaagtggcagcggctgacagaggaacagtgcctgtttagcgcctggctgtccgagaaagaggacgccgtcaacaagatccacaccaccggcttcaaggatcagaatgagatgctgagcagcctgcagaaactggccgtgctgaaggctgacctggaaaagaaaaagcagtccatgggcaagctgtactccctgaagcaggacctgctgagcacactgaagaacaagagcgtgacccagaaaaccgaggcctggctggacaacttcgccagatgctgggacaacctggtgcagaagctggaaaagtccaccgctcagatcagccaagagatcagctacgtgcccagcacatacctgaccgagatcacccatgtgtcacaggccctgcttgaggtggaacagctgctgaacgcccctgatctgtgcgccaaggacttcgaggatctgttcaagcaagaggaaagcctgaagaatatcaaggactctctgcagcagtccagcggccggatcgatatcatccacagcaagaaaaccgccgcactgcagtccgccacacctgtggaaagagtgaagctgcaagaagctctgagccagctggacttccagtgggagaaagtgaacaagatgtacaaggaccggcagggcagattcgaccgcagcgttgaaaagtggcggcggttccactacgacatcaagatcttcaaccagtggctgaccgaggccgaacagttcctgagaaagacacagatccccgagaactgggagcacgccaagtacaagtggtatctgaaagaactgcaggacggcatcggccagaggcagacagtcgtcagaacactgaatgccaccggcgaggaaatcatccagcagagcagcaagaccgacgccagcatcctgcaagagaagctgggaagcctgaacctgcggtggcaagaagtgtgcaagcagctgtccgaccggaagaagagactcgaggaacagcccgatctggcccctggcctgacaacaattggagctagccccacacagaccgtgacactggttacacagcccgtggtcaccaaagagacagccatcagcaaactggaaatgccctctagcctgatgctcgaagtgcccgcactggccgacttcaatagagcctggaccgagctgacagattggctgagcctcctggaccaagtgatcaagagccagagagtgatggtcggagatctcgaggacatcaacgagatgatcattaagcagaaagccaccatgcaggatctcgagcagagaaggcctcagctcgaagaactgatcaccgccgctcagaatctgaaaaacaagaccagcaatcaagaggcccggaccatcatcaccgaccggatcgagagaatccagaatcagtgggacgaagtccaagagcatctgcagaatcggcggcagcagctcaacgaaatgctgaaggatagcacccagtggctcgaggccaaagaagaggcagaacaggtcctcggacaggctagggccaagctggaatcttggaaagagggcccctacaccgtcgacgccatccagaagaagatcaccgagacaaagcagctggccaaggacctgagacagtggcagaccaatgtggacgtggccaacgatctggctctgaagctgctgagggactacagcgccgacgacaccagaaaggtgcacatgatcacagagaacatcaacgcctcttggcggagcatccacaagcgggtgtcagagagagaggccgctctcgaggaaacccacagactgcttcagcagttccctctggacctcgaaaagttcctggcctggctcacagaggccgagacaacagctaacgtcctccaggatgccaccagaaaagagagactgctcgaggactccaagggcgtcaaagaactcatgaagcagtggcaggatctgcagggcgaaatcgaggcccacaccgacgtgtaccacaacctggacgagaactcccagaagatcctgcggagcctggaaggcagtgatgatgctgtgctcctccagcggagactggacaacatgaacttcaagtggagcgagctgcgcaagaagtccctgaacatcagaagccacctggaagccagcagcgaccagtggaaaagactgcatctgtccctgcaagaactgctcgtgtggctgcagctcaaggacgacgaactgagcagacaggcccctatcggcggagattttcccgccgtgcagaaacagaacgacgtgcacagagccttcaagcgcgagctgaaaacaaaagaacccgtcatcatgagcaccctggaaaccgtgcggatctttctgaccgagcagcccctggaaggactggaaaagctgtaccaagagcctcgcgagctgcctcctgaagaaagggcccagaatgtgacccggctgctgagaaagcaggccgaggaagtgaataccgaatgggagaagctcaatctgcactccgccgactggcagcggaagatcgatgagacactggaacggctgcgggaattgcaagaggccaccgatgagctggacctgaagctgagacaggccgaagtcatcaaaggatcctggcagcctgtgggcgacctgctcatcgatagtctgcaggaccacctcgagaaagtcaaagccctgcggggagagatcgccccactgaaagaaaacgtgtcccacgtgaacgacctggccagacagctgacaacactgggcatccagctgagcccctacaacctgtccacacttgaggacctgaacacccggtggaaactgctccaagtggccgtggaagatagagtgcggcagctgcacgaagcccacagagattttggaccagccagccagcacttcctgagcacctcagttcaaggcccctgggagagagctatcagccctaacaaggtgccctactacatcaaccacgagacacagaccacctgttgggatcaccccaagatgaccgaactgtatcagtccctggccgatctgaacaatgtgcggttcagcgcctaccggaccgctatgaagctgcgaaggctgcagaaagctctgtgcctggacctcctgtctctgagcgctgcttgtgatgccctggaccagcataatctgaagcagaacgaccagcctatggacatcctccagatcatcaattgcctgaccaccatctacgaccgcctggaacaagaacacaacaacctcgtgaacgtgcccctgtgcgtggacatgtgcctgaattggctgctgaatgtgtacgacaccggccgcacaggcagaatcagagtgctgtccttcaagaccggcatcatctccctgtgcaaggcccatcttgaggacaagtaccgctacctgttcaaacaggtggcctccagcaccggcttttgcgaccaaagaaggctgggactgctcctgcacgacagcattcagatccctagacagctgggcgaagtggcctcctttggcggctctaatatcgagcctagcgtgcggagctgcttccagttcgccaacaacaagcccgagatcgaggctgccctgttcctggactggatgagactggaaccccagagcatggtctggctgccagtgctgcatagagtggccgctgccgaaacagccaagcaccaggccaagtgcaacatctgcaaagagtgccccatcatcggcttccggtacagatctctgaagcacttcaactacgatatctgccagagctgtttcttcagcggcagagtcgccaagggccacaaaatgcactaccccatggtggaatactgc

SEQ ID NO: 28 - protein, 1821 aa, synthetic construct
MLWWEEVEDCYEREDVQKKTFTKWVNAQFSKFGKQHIENLFSDLQDGRRLLDLLEGLTGQKLPKEKGSTRVHALNNVNKALRVLQNNNVDLVNIGSTDIVDGNHKLTLGLIWNIILHWQVKNVMKNIMAGLQQTNSEKILLSWVRQSTRNYPQVNVINFTTSWSDGLALNALIHSHRPDLFDWNSVVCQQSATQRLEHAFNIARYQLGIEKLLDPEDVDTTYPDKKSILMYITSLFQVLPQQVSIEAIQEVEMLPRPPKVTKEEHFQLHHQMHYSQQITVSLAQGYERTSSPKPRFKSYAYTQAAYVTTSDPTRSPFPSQHLEAPEDKSFGSSLMESEVNLDRYQTALEEVLSWLLSAEDTLQAQGEISNDVEVVKDQFHTHEGYMMDLTAHQGRVGNILQLGSKLIGTGKLSEDEETEVQEQMNLLNSRWECLRVASMEKQSNLHRVLMDLQNQKLKELNDWLTKTEERTRKMEEEPLGPDLEDLKRQVQQHKVLQEDLEQEQVRVNSLTHMVVVVDESSGDHATAALEEQLKVLGDRWANICRWTEDRWVLLQDILLKWQRLTEEQCLFSAWLSEKEDAVNKIHTTGFKDQNEMLSSLQKLAVLKADLEKKKQSMGKLYSLKQDLLSTLKNKSVTQKTEAWLDNFARCWDNLVQKLEKSTAQISQEISYVPSTYLTEITHVSQALLEVEQLLNAPDLCAKDFEDLFKQEESLKNIKDSLQQSSGRIDIIHSKKTAALQSATPVERVKLQEALSQLDFQWEKVNKMYKDRQGRFDRSVEKWRRFHYDIKIFNQWLTEAEQFLRKTQIPENWEHAKYKWYLKELQDGIGQRQTVVRTLNATGEEIIQQSSKTDASILQEKLGSLNLRWQEVCKQLSDRKKRLEEQPDLAPGLTTIGASPTQTVTLVTQPVVTKETAISKLEMPSSLMLEVPALADFNRAWTELTDWLSLLDQVIKSQRVMVGDLEDINEMIIKQKATMQDLEQRRPQLEELITAAQNLKNKTSNQEARTIITDRIERIQNQWDEVQEHLQNRRQQLNEMLKDSTQWLEAKEEAEQVLGQARAKLESWKEGPYTVDAIQKKITETKQLAKDLRQWQTNVDVANDLALKLLRDYSADDTRKVHMITENINASWRSIHKRVSEREAALEETHRLLQQFPLDLEKFLAWLTEAETTANVLQDATRKERLLEDSKGVKELMKQWQDLQGEIEAHTDVYHNLDENSQKILRSLEGSDDAVLLQRRLDNMNFKWSELRKKSLNIRSHLEASSDQWKRLHLSLQELLVWLQLKDDELSRQAPIGGDFPAVQKQNDVHRAFKRELKTKEPVIMSTLETVRIFLTEQPLEGLEKLYQEPRELPPEERAQNVTRLLRKQAEEVNTEWEKLNLHSADWQRKIDETLERLRELQEATDELDLKLRQAEVIKGSWQPVGDLLIDSLQDHLEKVKALRGEIAPLKENVSHVNDLARQLTTLGIQLSPYNLSTLEDLNTRWKLLQVAVEDRVRQLHEAHRDFGPASQHFLSTSVQGPWERAISPNKVPYYINHETQTTCWDHPKMTELYQSLADLNNVRFSAYRTAMKLRRLQKALCLDLLSLSAACDALDQHNLKQNDQPMDILQIINCLTTIYDRLEQEHNNLVNVPLCVDMCLNWLLNVYDTGRTGRIRVLSFKTGIISLCKAHLEDKYRYLFKQVASSTGFCDQRRLGLLLHDSIQIPRQLGEVASFGGSNIEPSVRSCFQFANNKPEIEAALFLDWMRLEPQSMVWLPVLHRVAAAETAKHQAKCNICKECPIIGFRYRSLKHFNYDICQSCFFSGRVAKGHKMHYPMVEYC

SEQ ID NO: 29 - DNA, 5463 bp, synthetic construct
  source 1..5463: mol_type = other DNA
atgctttggtgggaagaagtcgaggactgctacgagcgcgaggacgtgcagaagaaaaccttcaccaaatgggtcaacgcccagttcagcaagttcggcaagcagcacatcgagaacctgttcagcgacctgcaggatggcagaaggctgctggatctgctggaaggcctgacaggacagaagctgcccaaagagaagggcagcacaagagtgcacgccctgaacaacgtgaacaaggccctgagagtgctgcagaacaacaacgtggacctggtcaacatcggcagcaccgacatcgtggacggcaaccacaaactgaccctgggcctgatctggaacatcatcctgcactggcaagtgaagaacgtgatgaagaacatcatggccggcctgcagcagaccaacagcgagaagattctgctgagctgggtccgacagagcacccggaattaccctcaagtgaacgtgatcaacttcaccacctcttggagcgacggactggccctgaatgccctgatccacagccacagacctgacctgttcgactggaacagcgtcgtgtgtcagcagagcgccacacagaggctggaacacgccttcaatatcgccagataccagctgggcatcgagaaactgctggaccccgaggatgtggacaccacctatcctgacaagaaatccatcctcatgtacatcaccagcctgttccaggtgctgccccagcaggtttccatcgaggccattcaagaggtcgagatgctgcccagacctcctaaagtgaccaaagaggaacacttccagctgcaccaccagatgcactactctcagcagatcaccgtgtctctggcccagggctacgagagaacaagcagccccaagcctcggttcaagagctacgcctatacacaggccgcctacgtgaccaccagcgatcctacaagaagcccatttcctagccagcacctcgaggcccctgaggataagagctttggcagcagcctgatggaaagcgaagtgaacctggaccgctaccagacagccctggaagaggttctgagctggctgctgtctgccgaggatacactgcaggctcagggcgagatcagcaacgacgtggaagtggtcaaggaccagtttcacacccacgagggctacatgatggacctgacagcccaccagggcagagtgggcaatattctgcagctgggctccaagctgatcggcacaggcaagctgagcgaggacgaagagacagaggtgcaagagcagatgaacctgctgaacagcagatgggagtgtctgagagtggccagcatggaaaagcagagcaacctgcaccgggtgctgatggatctccagaaccagaagctgaaagagctgaacgactggctgaccaagaccgaggaacggacccggaagatggaagaggaacctctgggacccgacctggaagatctgaaaagacaggtgcagcagcataaggtgctgcaagaggacctcgagcaagagcaagtgcgcgtgaacagcctgacacacatggtggtggtcgtggatgagagcagcggagatcatgccacagccgctctggaagaacagctgaaggtgctgggagacagatgggccaatatctgccggtggaccgaggatagatgggtgctgctccaggacatcctgctgaagtggcagcggctgacagaggaacagtgcctgtttagcgcctggctgtccgagaaagaggacgccgtcaacaagatccacaccaccggcttcaaggatcagaatgagatgctgagcagcctgcagaaactggccgtgctgaaggctgacctggaaaagaaaaagcagtccatgggcaagctgtactccctgaagcaggacctgctgagcacactgaagaacaagagcgtgacccagaaaaccgaggcctggctggacaacttcgccagatgctgggacaacctggtgcagaagctggaaaagtccaccgctcagatcagtcagcagcccgatctggcacctggcctgacaacaattggcgctagccccacacagaccgtgacactggttacacagcccgtggtcaccaaagagacagccatcagcaaactggaaatgccctctagcctgatgctcgaagtgcccgagatctcctacgtgcccagcacatacctgaccgagatcacccatgtgtcacaggccctgcttgaggtggaacagctgctgaacgcccctgatctgtgcgccaaggacttcgaggatctgttcaagcaagaggaaagcctgaagaatatcaaggactctctgcagcagtccagcggccggatcgatatcatccacagcaagaaaaccgccgcactgcagtccgccacacctgtggaaagagtgaagctgcaagaagctctgagccagctggacttccagtgggagaaagtgaacaagatgtacaaggaccggcagggcagattcgaccgcagcgttgaaaagtggcggcggttccactacgacatcaagatcttcaaccagtggctgaccgaggccgaacagttcctgagaaagacacagatccccgagaactgggagcacgccaagtacaagtggtatctgaaagaactgcaggacggcatcggccagaggcagacagtcgtcagaacactgaatgccaccggcgaggaaatcatccagcagagcagcaagaccgacgccagcatcctgcaagagaagctgggaagcctgaacctgcggtggcaagaagtgtgcaagcagctgtccgaccggaagaagagactggaagaagccctggccgacttcaacagagcctggaccgaactgaccgattggctgagcctcctggaccaagtgatcaagagccagagagtgatggtcggagatcttgaggacatcaacgagatgatcattaagcagaaagccaccatgcaggatctcgagcagagaaggcctcagctcgaggaactgatcaccgccgctcagaatctgaaaaacaagaccagcaatcaagaggcccggaccatcatcaccgaccggatcgagagaatccagaatcagtgggacgaagtccaagagcatctgcagaatcggcggcagcagctcaacgaaatgctgaaggatagcacccagtggctcgaggccaaagaagaggcagaacaggtcctcggacaggctagggccaagctggaatcttggaaagagggcccctacaccgtcgacgccatccagaagaagatcaccgagacaaagcagctggccaaggacctgagacagtggcagaccaatgtggacgtggccaatgatctggctctgaagctgctgagggactacagcgccgacgacaccagaaaggtgcacatgatcacagagaacatcaacgcctcttggcggagcatccacaagcgggtgtcagagagagaggccgctctcgaggaaacccacagactgcttcagcagttccctctggacctcgaaaagttcctggcctggctcacagaggccgagacaacagctaacgtcctccaggatgccaccagaaaagagagactgctcgaggacagcaagggcgtcaaagaactcatgaagcagtggcaggatctgcagggcgaaatcgaggcccacaccgacgtgtaccacaacctggacgagaactcccagaagatcctgcggagcctggaaggcagtgatgatgctgtgctcctccagcggagactggacaacatgaacttcaagtggagcgagctgcgcaagaagtccctgaacatcagaagccacctggaagccagcagcgaccagtggaaaagactgcatctgtccctgcaagaactgctcgtgtggctgcagctcaaggacgacgaactgagcagacaggcccctatcggcggagattttcccgccgtgcagaaacagaacgacgtgcacagagccttcaagcgcgagctgaaaacaaaagaacccgtcatcatgagcaccctggaaaccgtgcggatctttctgaccgagcagcccctggaaggactcgagaagctgtaccaagagcctcgcgaactgcctcctgaagaaagggcccagaacgtgacccggctgctgagaaaacaggccgaggaagtgaacaccgagtgggaaaagctgaatctgcactccgccgactggcagcggaagatcgatgagacactggaacggctgcgggaattgcaagaggccaccgatgagctggacctgaagctgagacaggccgaagtcatcaaaggatcctggcagcctgtgggcgacctgctcatcgatagtctgcaggaccacctcgagaaagtcaaagccctgcggggagagatcgccccactgaaagaaaacgtgtcccacgtgaacgacctggccagacagctgacaacactgggcatccagctgagcccctacaacctgtccacacttgaggacctgaacacccggtggaaactgctccaagtggccgtggaagatagagtgcggcagctgcacgaagcccacagagattttggaccagccagccagcacttcctgagcacctcagttcaaggcccctgggagagagctatcagccctaacaaggtgccctactacatcaaccacgagacacagaccacctgttgggatcaccccaagatgaccgagctgtatcagagtctggctgatctgaacaatgtgcggttcagcgcctaccggaccgctatgaagctgcgaaggctgcagaaagctctgtgcctggacctcctgtctctgagcgctgcttgtgatgccctggaccagcataatctgaagcagaacgaccagcctatggacatcctccagatcatcaattgcctgaccaccatctacgaccgcctggaacaagaacacaacaacctcgtgaacgtgcccctgtgcgtggacatgtgcctgaattggctgctgaatgtgtacgacaccggccgcacaggcagaatcagagtgctgtccttcaagaccggcatcatctccctgtgcaaggcccacctcgaagataagtaccgctacctgttcaaacaggtggcctccagcaccggcttttgcgaccaaagaaggctgggactgctcctgcacgacagcattcagatccctagacagctgggcgaagtggcctcctttggcggctctaatatcgagcctagcgtgcggagctgcttccagttcgccaacaacaagcctgagatcgaggctgccctgttcctggactggatgagactcgagcctcagagcatggtctggctgccagtgctgcatagagtggccgctgccgaaacagccaagcaccaggccaagtgcaacatctgcaaagagtgccccatcatcggcttccggtacagatctctgaagcacttcaactacgatatctgccagagctgtttcttcagcggcagagtcgccaagggccacaaaatgcactaccccatggtggaatactgc

SEQ ID NO: 30 - protein, 1821 aa, synthetic construct
MLWWEEVEDCYEREDVQKKTFTKWVNAQFSKFGKQHIENLFSDLQDGRRLLDLLEGLTGQKLPKEKGSTRVHALNNVNKALRVLQNNNVDLVNIGSTDIVDGNHKLTLGLIWNIILHWQVKNVMKNIMAGLQQTNSEKILLSWVRQSTRNYPQVNVINFTTSWSDGLALNALIHSHRPDLFDWNSVVCQQSATQRLEHAFNIARYQLGIEKLLDPEDVDTTYPDKKSILMYITSLFQVLPQQVSIEAIQEVEMLPRPPKVTKEEHFQLHHQMHYSQQITVSLAQGYERTSSPKPRFKSYAYTQAAYVTTSDPTRSPFPSQHLEAPEDKSFGSSLMESEVNLDRYQTALEEVLSWLLSAEDTLQAQGEISNDVEVVKDQFHTHEGYMMDLTAHQGRVGNILQLGSKLIGTGKLSEDEETEVQEQMNLLNSRWECLRVASMEKQSNLHRVLMDLQNQKLKELNDWLTKTEERTRKMEEEPLGPDLEDLKRQVQQHKVLQEDLEQEQVRVNSLTHMVVVVDESSGDHATAALEEQLKVLGDRWANICRWTEDRWVLLQDILLKWQRLTEEQCLFSAWLSEKEDAVNKIHTTGFKDQNEMLSSLQKLAVLKADLEKKKQSMGKLYSLKQDLLSTLKNKSVTQKTEAWLDNFARCWDNLVQKLEKSTAQISQQPDLAPGLTTIGASPTQTVTLVTQPVVTKETAISKLEMPSSLMLEVPEISYVPSTYLTEITHVSQALLEVEQLLNAPDLCAKDFEDLFKQEESLKNIKDSLQQSSGRIDIIHSKKTAALQSATPVERVKLQEALSQLDFQWEKVNKMYKDRQGRFDRSVEKWRRFHYDIKIFNQWLTEAEQFLRKTQIPENWEHAKYKWYLKELQDGIGQRQTVVRTLNATGEEIIQQSSKTDASILQEKLGSLNLRWQEVCKQLSDRKKRLEEALADFNRAWTELTDWLSLLDQVIKSQRVMVGDLEDINEMIIKQKATMQDLEQRRPQLEELITAAQNLKNKTSNQEARTIITDRIERIQNQWDEVQEHLQNRRQQLNEMLKDSTQWLEAKEEAEQVLGQARAKLESWKEGPYTVDAIQKKITETKQLAKDLRQWQTNVDVANDLALKLLRDYSADDTRKVHMITENINASWRSIHKRVSEREAALEETHRLLQQFPLDLEKFLAWLTEAETTANVLQDATRKERLLEDSKGVKELMKQWQDLQGEIEAHTDVYHNLDENSQKILRSLEGSDDAVLLQRRLDNMNFKWSELRKKSLNIRSHLEASSDQWKRLHLSLQELLVWLQLKDDELSRQAPIGGDFPAVQKQNDVHRAFKRELKTKEPVIMSTLETVRIFLTEQPLEGLEKLYQEPRELPPEERAQNVTRLLRKQAEEVNTEWEKLNLHSADWQRKIDETLERLRELQEATDELDLKLRQAEVIKGSWQPVGDLLIDSLQDHLEKVKALRGEIAPLKENVSHVNDLARQLTTLGIQLSPYNLSTLEDLNTRWKLLQVAVEDRVRQLHEAHRDFGPASQHFLSTSVQGPWERAISPNKVPYYINHETQTTCWDHPKMTELYQSLADLNNVRFSAYRTAMKLRRLQKALCLDLLSLSAACDALDQHNLKQNDQPMDILQIINCLTTIYDRLEQEHNNLVNVPLCVDMCLNWLLNVYDTGRTGRIRVLSFKTGIISLCKAHLEDKYRYLFKQVASSTGFCDQRRLGLLLHDSIQIPRQLGEVASFGGSNIEPSVRSCFQFANNKPEIEAALFLDWMRLEPQSMVWLPVLHRVAAAETAKHQAKCNICKECPIIGFRYRSLKHFNYDICQSCFFSGRVAKGHKMHYPMVEYC

SEQ ID NO: 31 - DNA, 5949 bp, synthetic construct
  source 1..5949: mol_type = other DNA
atgctttggtgggaagaagtcgaggactgctacgagcgcgaggacgtgcagaagaaaaccttcaccaaatgggtcaacgcccagttcagcaagttcggcaagcagcacatcgagaacctgttcagcgacctgcaggatggcagaaggctgctggatctgctggaaggcctgacaggacagaagctgcccaaagagaagggcagcacaagagtgcacgccctgaacaacgtgaacaaggccctgagagtgctgcagaacaacaacgtggacctggtcaacatcggcagcaccgacatcgtggacggcaaccacaaactgaccctgggcctgatctggaacatcatcctgcactggcaagtgaagaacgtgatgaagaacatcatggccggcctgcagcagaccaacagcgagaagattctgctgagctgggtccgacagagcacccggaattaccctcaagtgaacgtgatcaacttcaccacctcttggagcgacggactggccctgaatgccctgatccacagccacagacctgacctgttcgactggaacagcgtcgtgtgtcagcagagcgccacacagaggctggaacacgccttcaatatcgccagataccagctgggcatcgagaaactgctggaccccgaggatgtggacaccacctatcctgacaagaaatccatcctcatgtacatcaccagcctgttccaggtgctgccccagcaggtttccatcgaggccattcaagaggtcgagatgctgcccagacctcctaaagtgaccaaagaggaacacttccagctgcaccaccagatgcactactctcagcagatcaccgtgtctctggcccagggctacgagagaacaagcagccccaagcctcggttcaagagctacgcctatacacaggccgcctacgtgaccaccagcgatcctacaagaagcccatttcctagccagcacctcgaggcccctgaggataagagctttggcagcagcctgatggaaagcgaagtgaacctggaccgctaccagacagccctggaagaggttctgagctggctgctgtctgccgaggatacactgcaggctcagggcgagatcagcaacgacgtggaagtggtcaaggaccagtttcacacccacgagggctacatgatggacctgacagcccaccagggcagagtgggcaatattctgcagctgggctccaagctgatcggcacaggcaagctgagcgaggacgaagagacagaggtgcaagagcagatgaacctgctgaacagcagatgggagtgtctgagagtggccagcatggaaaagcagagcaacctgcaccgggtgctgatggatctccagaaccagaagctgaaagagctgaacgactggctgaccaagaccgaggaacggacccggaagatggaagaggaacctctgggacccgacctggaagatctgaaaagacaggtgcagcagcataaggtgctgcaagaggacctcgagcaagagcaagtgcgcgtgaacagcctgacacacatggtggtggtcgtggatgagagcagcggagatcatgccacagccgctctggaagaacagctgaaggtgctgggagacagatgggccaatatctgccggtggaccgaggatagatgggtgctgctccaggacatcctgctgaagtggcagcggctgacagaggaacagtgcctgtttagcgcctggctgtccgagaaagaggacgccgtcaacaagatccacaccaccggcttcaaggatcagaatgagatgctgagcagcctgcagaaactggccgtgctgaaggctgacctggaaaagaaaaagcagtccatgggcaagctgtactccctgaagcaggacctgctgagcacactgaagaacaagagcgtgacccagaaaaccgaggcctggctggacaacttcgccagatgctgggacaacctggtgcagaagctggaaaagtccaccgctcaggaaaccgagattgccgtgcaggccaagcagccagatgtggaagagatcctgagcaagggacagcacctgtacaaagagaaacccgccactcagcccgtgaagagaaagctcgaggatctgagcagcgagtggaaggccgtgaacagactcctgcaagagctgagagccaaacagcccgatctggcccctggcctgacaacaattggagctagccccacacagaccgtgacactggttacacagcccgtggtcaccaaagagacagccatcagcaaactggaaatgccctctagcctgatgctcgaagtgcccgctctggccgacttcaatagagcctggaccgagctgacagattggctgagcctcctggaccaagtgatcaagagccagagagtgatggtcggagatctcgaggacatcaacgagatgatcattaagcagaaagccaccatgcaggatctcgaacagagaaggccccagctcgaagaactgatcaccgccgcacagaacctgaaaaacaagaccagcaatcaagaagccaggaccatcatcaccgaccggatcgagcggatccagaatcagtgggacgaagtgcaagaacatctgcagaaccggcggcagcagctcaacgaaatgctgaaggatagcacccagtggctggaagccaaagaagaggccgagcaggttctgggacaagccagagccaagctggaatcctggaaagagggaccctacaccgtcgacgccatccagaagaagatcaccgagacaaagcagctggccaaggacctgagacagtggcagaccaatgtggacgtggccaacgatctggctctgaagctgctgagggactacagcgccgacgacaccagaaaggtgcacatgatcacagagaacatcaacgcctcttggcggagcatccacaagcgggtgtccgaaagagaggccgctctcgaggaaacccacagactgcttcagcagttccctctggatctcgagaagttcctggcttggctgaccgaggccgaaaccacagctaacgttctgcaggacgccacacggaaagagagactgctcgaggactccaagggcgtcaaagaactcatgaagcagtggcaggatctgcagggcgaaatcgaggcccacaccgacgtgtaccacaacctggacgagaactcccagaagatcctgcggagcctggaaggcagtgatgatgctgtgctcctccagcggagactggacaacatgaacttcaagtggagcgagctgcgcaagaagtccctgaacatcagaagccacctggaagcctccagcgaccagtggaaaagactgcatctgtccctgcaagaactgctcgtgtggctgcagctcaaggacgacgaactgagcagacaggcccctatcggcggagattttcccgccgtgcagaaacagaacgacgtgcacagagccttcaagcgcgagctgaaaacaaaagaacccgtcatcatgagcaccctggaaaccgtgcggatcttcctgacagagcagcctctcgaaggactggaaaagctgtaccaagagcctcgcgagctgcctcctgaagaaagggcccagaatgtgacccggctgctgagaaagcaggccgaggaagtgaataccgagtgggagaaactgaacctgcactccgccgactggcagcggaagatcgatgagacactggaacggctgcgggaactgcaagaagctaccgacgagctggacctgaagctgagacaggccgaagtcatcaaaggatcctggcagcctgtgggcgacctgctcatcgattctctgcaggatcacctcgagaaagtgaaggccctgcggggagaaatcgcccctctgaaagaaaacgtgtcccacgtgaacgacctggccagacagctgacaacactgggcatccagctgagcccctacaacctgtccacactcgaggacctgaacacccggtggaaactgctccaagtggccgtggaagatagagtgcggcagctgcacgaagcccacagagattttggaccagccagccagcacttcctgagcacctcagttcaaggcccctgggagagagctatcagccctaacaaggtgccctactacatcaaccacgagacacagaccacctgttgggatcaccccaagatgaccgaactgtatcagtccctggccgatctgaacaatgtgcggttcagcgcctaccggaccgctatgaagctgcgaaggctgcagaaagctctgtgcctggacctgctctctctgagcgctgcttgtgatgccctggaccagcataatctgaagcagaacgaccagcctatggacatcctccagatcatcaattgcctgaccaccatctacgaccgcctggaacaagaacacaacaacctcgtgaacgtgcccctgtgcgtggacatgtgcctgaattggctgctgaacgtgtacgacaccggccggacaggcagaatcagagtgctgtccttcaagaccggcatcatctccctgtgcaaggcccaccttgaggacaagtaccgctacctgttcaaacaggtggcctccagcaccggcttttgcgaccaaagaaggctgggactgctcctgcacgacagcattcagatccctagacagctgggcgaagtggcctctttcggcggctctaatatcgagcctagcgtgcggagctgcttccagttcgccaacaacaagcccgagatcgaggctgccctgttcctggactggatgagactggaaccccagagcatggtctggctgcctgtgctgcatagagtggccgctgccgaaacagccaagcaccaggccaagtgcaacatctgtaaagagtgccccatcatcggcttccggtacagaagcctgaagcacttcaactacgatatctgccagagctgtttcttcagcggcagagtcgccaagggccacaaaatgcactaccccatggtggaatactgcacccctaccacctccggcgaagatgtgcgggattttgccaaggtgctcaaaaacaagttccggaccaagcgctacttcgctaagcaccccagaatgggctatctgcccgtgcagacagtgctcgagggcgacaatatggaaacccctgtgacactgatcaatttctggcccgtggacagcgcccctgctagttctcctcaactgagccacgacgatacccacagcaggatcgagcactacgccagcagactggccgagatggaaaacagcaacggcagctacctgaacgatagcatcagccccaacgagagcatcgacgacgagcatctgctgatccagcactactgtcagagcctgaaccaggactccccactgagccagcctagaagccctgcacagatcctgatcagcctggaatccgaggaaaggggcgagctggaaagaatcctggccgacctcgaagaggaaaaccggaacctgcaggcagagtacgacagactgaagcagcagcacgagcacaagggactgtctccactgccatctccacctgaaatgatgcccacctctccacagagccccagagatgccgagctgattgccgaagccaaactcctcagacagcacaaaggcagactggaagcccggatgcagattcttgaggaccacaacaaacagctcgagtctcagctgcacagactgagacagctgcttgagcaacctcaggctgaggccaaagtgaacggcaccacagtgtctagcccctccactagtctgcagcggagcgattctagccagccaatgctcctgagagtcgtgggctctcagaccagcgatagcatgggcgaagaggatctgctcagccctcctcaggatacaagcaccggcctggaagaagttatggaacagctcaacaacagcttccccagcagcagaggcagaaacacccctggcaagcccatgcgcgaagataccatg

SEQ ID NO: 32 - protein, 1983 aa, Homo sapiens
MLWWEEVEDCYEREDVQKKTFTKWVNAQFSKFGKQHIENLFSDLQDGRRLLDLLEGLTGQKLPKEKGSTRVHALNNVNKALRVLQNNNVDLVNIGSTDIVDGNHKLTLGLIWNIILHWQVKNVMKNIMAGLQQTNSEKILLSWVRQSTRNYPQVNVINFTTSWSDGLALNALIHSHRPDLFDWNSVVCQQSATQRLEHAFNIARYQLGIEKLLDPEDVDTTYPDKKSILMYITSLFQVLPQQVSIEAIQEVEMLPRPPKVTKEEHFQLHHQMHYSQQITVSLAQGYERTSSPKPRFKSYAYTQAAYVTTSDPTRSPFPSQHLEAPEDKSFGSSLMESEVNLDRYQTALEEVLSWLLSAEDTLQAQGEISNDVEVVKDQFHTHEGYMMDLTAHQGRVGNILQLGSKLIGTGKLSEDEETEVQEQMNLLNSRWECLRVASMEKQSNLHRVLMDLQNQKLKELNDWLTKTEERTRKMEEEPLGPDLEDLKRQVQQHKVLQEDLEQEQVRVNSLTHMVVVVDESSGDHATAALEEQLKVLGDRWANICRWTEDRWVLLQDILLKWQRLTEEQCLFSAWLSEKEDAVNKIHTTGFKDQNEMLSSLQKLAVLKADLEKKKQSMGKLYSLKQDLLSTLKNKSVTQKTEAWLDNFARCWDNLVQKLEKSTAQETEIAVQAKQPDVEEILSKGQHLYKEKPATQPVKRKLEDLSSEWKAVNRLLQELRAKQPDLAPGLTTIGASPTQTVTLVTQPVVTKETAISKLEMPSSLMLEVPALADFNRAWTELTDWLSLLDQVIKSQRVMVGDLEDINEMIIKQKATMQDLEQRRPQLEELITAAQNLKNKTSNQEARTIITDRIERIQNQWDEVQEHLQNRRQQLNEMLKDSTQWLEAKEEAEQVLGQARAKLESWKEGPYTVDAIQKKITETKQLAKDLRQWQTNVDVANDLALKLLRDYSADDTRKVHMITENINASWRSIHKRVSEREAALEETHRLLQQFPLDLEKFLAWLTEAETTANVLQDATRKERLLEDSKGVKELMKQWQDLQGEIEAHTDVYHNLDENSQKILRSLEGSDDAVLLQRRLDNMNFKWSELRKKSLNIRSHLEASSDQWKRLHLSLQELLVWLQLKDDELSRQAPIGGDFPAVQKQNDVHRAFKRELKTKEPVIMSTLETVRIFLTEQPLEGLEKLYQEPRELPPEERAQNVTRLLRKQAEEVNTEWEKLNLHSADWQRKIDETLERLRELQEATDELDLKLRQAEVIKGSWQPVGDLLIDSLQDHLEKVKALRGEIAPLKENVSHVNDLARQLTTLGIQLSPYNLSTLEDLNTRWKLLQVAVEDRVRQLHEAHRDFGPASQHFLSTSVQGPWERAISPNKVPYYINHETQTTCWDHPKMTELYQSLADLNNVRFSAYRTAMKLRRLQKALCLDLLSLSAACDALDQHNLKQNDQPMDILQIINCLTTIYDRLEQEHNNLVNVPLCVDMCLNWLLNVYDTGRTGRIRVLSFKTGIISLCKAHLEDKYRYLFKQVASSTGFCDQRRLGLLLHDSIQIPRQLGEVASFGGSNIEPSVRSCFQFANNKPEIEAALFLDWMRLEPQSMVWLPVLHRVAAAETAKHQAKCNICKECPIIGFRYRSLKHFNYDICQSCFFSGRVAKGHKMHYPMVEYCTPTTSGEDVRDFAKVLKNKFRTKRYFAKHPRMGYLPVQTVLEGDNMETPVTLINFWPVDSAPASSPQLSHDDTHSRIEHYASRLAEMENSNGSYLNDSISPNESIDDEHLLIQHYCQSLNQDSPLSQPRSPAQILISLESEERGELERILADLEEENRNLQAEYDRLKQQHEHKGLSPLPSPPEMMPTSPQSPRDAELIAEAKLLRQHKGRLEARMQILEDHNKQLESQLHRLRQLLEQPQAEAKVNGTTVSSPSTSLQRSDSSQPMLLRVVGSQTSDSMGEEDLLSPPQDTSTGLEEVMEQLNNSFPSSRGRNTPGKPMREDTM

SEQ ID NO: 33 - protein, 14 aa, synthetic construct
GKPIPNPLLGLDST

(19) United States
(12) Patent Application Publication
(10) Pub. No.: US 2025/0099621 A1
(43) Pub. Date: Mar. 27, 2025

(54) SYSTEMIC ADMINISTRATION OF CIRCULAR RNA POLYNUCLEOTIDES ENCODING MUSCLE PROTEINS OR PROTEIN COMPLEXES

(71) Applicant: Orna Therapeutics, Inc., Watertown, MA (US)

(72) Inventors: Allen T. Horhota, Watertown, MA (US); Robert Alexander Wesselhoeft, Watertown, MA (US); Shobu Odate, Watertown, MA (US); Tatiana Fontelonga, Watertown, MA (US); Junghoon Yang, Watertown, MA (US)

(21) Appl. No.: 18/729,257

(22) PCT Filed: Jan. 20, 2023
(86) PCT No.: PCT/US2023/061018

§ 371 Date: Jul. 16, 2024

Related U.S. Application Data
(60) Provisional application No. 63/342,538, filed on May 16, 2022.
(60) Provisional application No. 63/301,931, filed on Jan. 21, 2022.

(51) Int. Cl. A61K 48/00; A61K 9/127; A61P 21/00; C07K 14/47; C12N 15/85

(52) U.S. Cl. CPC A61K 48/0058; A61K 9/127; A61K 48/0066; A61P 21/00; C07K 14/4716; C12N 15/85; C12N 2800/107

(57) ABSTRACT

Disclosed herein are lipid nanoparticle (LNP) compositions encapsulating nucleic acid constructs for systemically delivering muscle proteins or protein complexes to muscle tissues.

### CROSS REFERENCE TO RELATED APPLICATIONS

This application claims the benefit of, and priority to, U.S. Provisional Application No. 63/301,931, filed on Jan. 21, 2022; and U.S. Provisional Application No. 63/342,538, filed on May 16, 2022, the contents of each of which are hereby incorporated by reference in their entirety for all purposes

### SEQUENCE LISTING

This application contains a Sequence Listing which has been submitted electronically in XML format and is hereby incorporated by reference in its entirety. Said XML file, created on Jan. 19, 2022, is named OBS_020WO_SL.xml and is 197 kilobytes in size.

### BACKGROUND

Gene therapy involves the introduction of desired genetic information into host cells, whether directly in a patient's body in vivo, or by engineering cells that are then administered to the patient. Historically, in vivo gene therapy using viral vectors can result in an adverse immune response or undesirable random integration of genetic material into the host genome. Multiple alternative approaches using non-viral delivery such as lipid nanoparticles (LNP) have been developed to overcome limitations of viral vector systems.

Many LNP systems include an ionizable lipid that contributes to LNP uptake by target cells, as well as neutral lipid, helper lipid, and cholesterol components that contribute to stability, circulation time, and ability to release nucleic acid cargo in the target cell. A large variety of LNP systems have been designed that are primarily taken up by the liver, and several have been clinically validated for delivery of RNA therapeutic components such as siRNA for gene silencing or mRNA for protein production. In addition, many attempts have been made to target LNPs to organs other than the liver, but it is difficult to overcome the propensity of LNPs to be taken up by liver cells before they can reach another target tissue in sufficient quantities.

One particularly challenging tissue to reach via LNP delivery is muscle. While LNPs containing genetic material have been successfully delivered to muscle by direct injection resulting in protein expression, local intramuscular administration has extremely limited potential for therapies to treat muscle disorders such as muscular dystrophy, which ideally benefit from therapeutic protein expression in non-skeletal muscle such as cardiac and diaphragm tissue. We describe herein novel compositions and methods to systemically deliver nucleic acids to muscle via a non-viral LNP delivery vehicle, resulting in protein expression. Surprisingly, the compositions described herein do not require additional targeting moieties specific to muscle cells to achieve expression of muscle protein from the nucleic acid cargo.

Multiple types of nucleic acid constructs may be useful for therapeutic or prophylactic purposes, including DNA and RNA constructs. For example, RNA has been successfully used in human vaccines, as well as in gene therapy applications such as gene editing. mRNA is typically used to express a desired protein, such as a viral protein in a vaccine. To date, existing mRNA therapeutics use linear mRNA constructs, which can be difficult and expensive to manufacture reproducibly, and require special elements such as modified nucleotides, caps, and tails to prevent degradation and immunogenicity. Recently, circular RNA has been described for the design and production of RNA for gene therapy. Circular RNA can be particularly useful for in vivo applications, as it can have improved properties relating to manufacturing, purification, protein expression, formulation compatibility, and immunogenicity compared to traditional linear mRNA. The combination of properties of circular RNA with certain LNP delivery systems can result in expression of muscle proteins such as dystrophin in therapeutically relevant tissues, including cardiac muscle and diaphragm. Circular RNA is particularly advantageous in that it can be used to express large proteins that would not fit in a traditional viral vector such as AAV, or that would be difficult to manufacture as a traditional linear mRNA construct. In addition, when formulated in a suitable LNP, circular RNA may be dosed multiple times, potentially resulting in increased protein expression compared to a single dose. The invention described herein provides compositions and methods for systemically delivering nucleic acid constructs to muscle, resulting in protein expression in muscle tissue. In particular, compositions containing circular RNA may be useful for delivering therapeutic proteins to muscle for the treatment of diseases, such as Becker's disease, Duchenne Muscular Dystrophy (DMD), or Congenital Muscular Dystrophy type 1A (MDCIA).

### SUMMARY

Provided herein are pharmaceutical composition comprising a circular RNA polynucleotide encoding a muscle protein or protein complex, or a variant thereof, and a transfer vehicle comprising an ionizable lipid.

In some embodiments, the ionizable lipid is a compound of Formula (1):

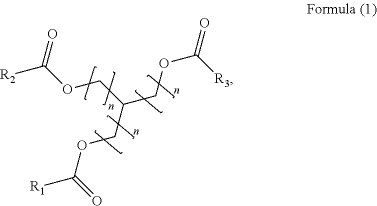

or a pharmaceutically acceptable salt thereof, wherein:
each n is independently an integer from 1-15;
R₁ and R2 are each independently selected from a group consisting of:

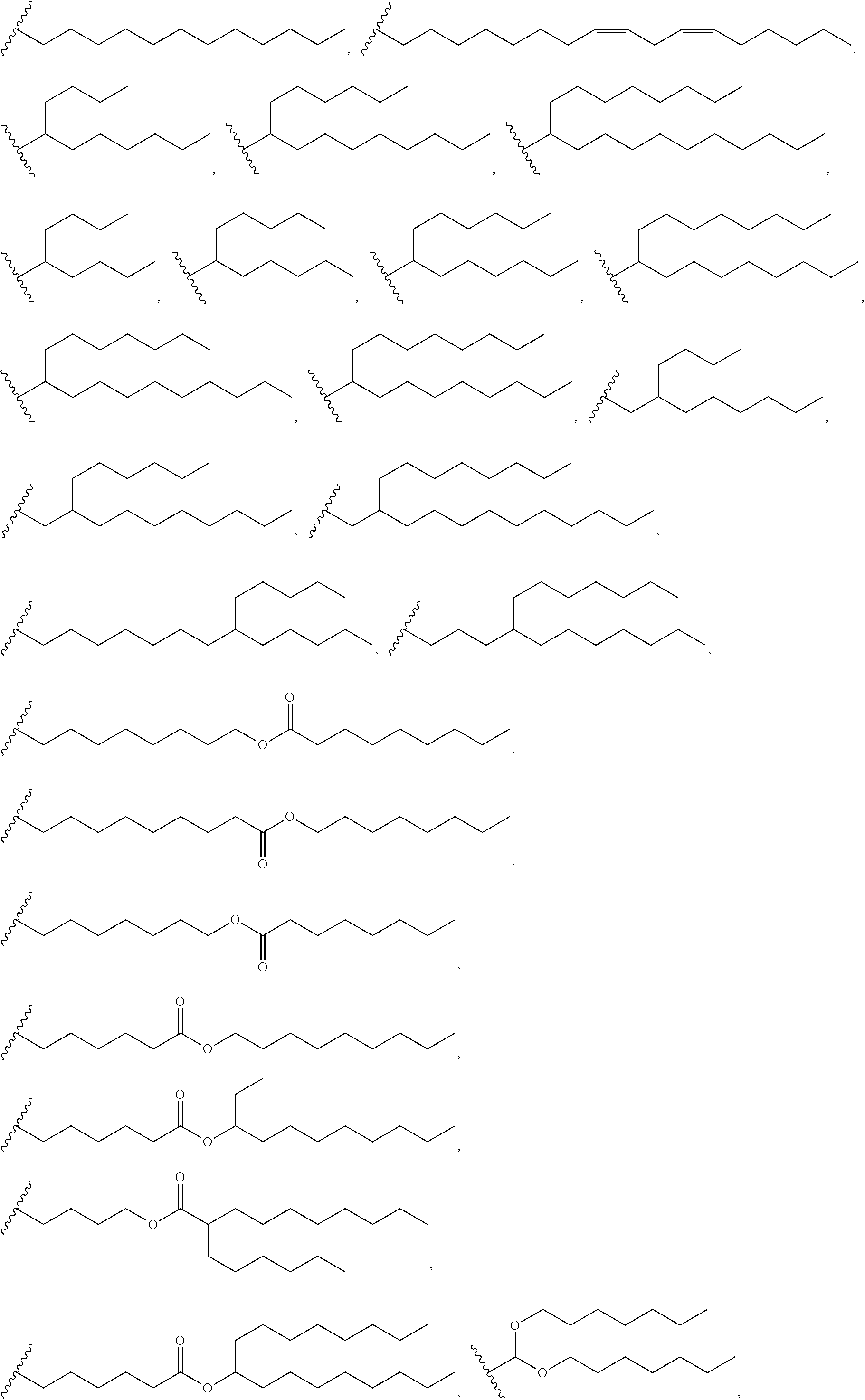

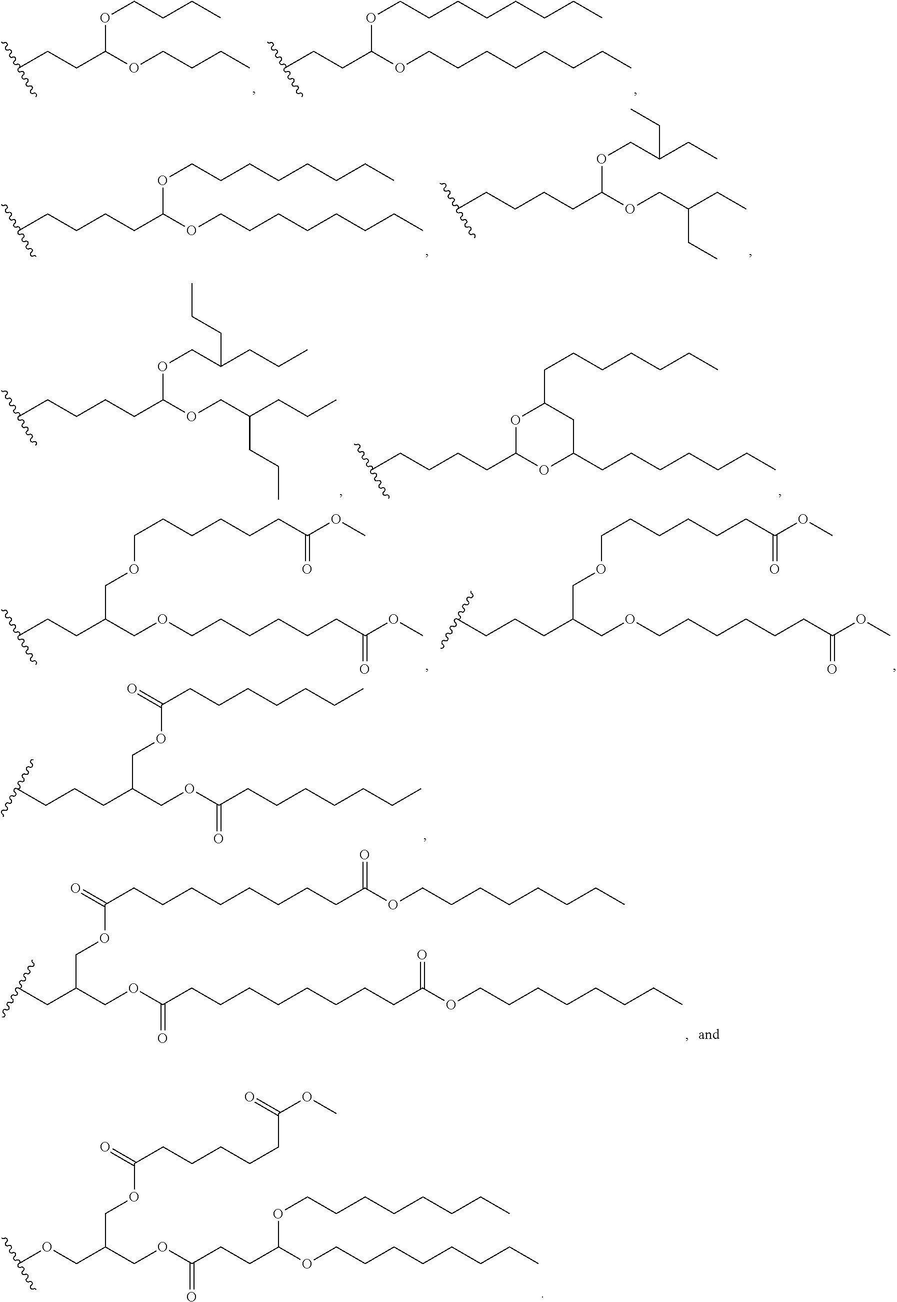

In some embodiments, R3 is selected from a group consisting of:

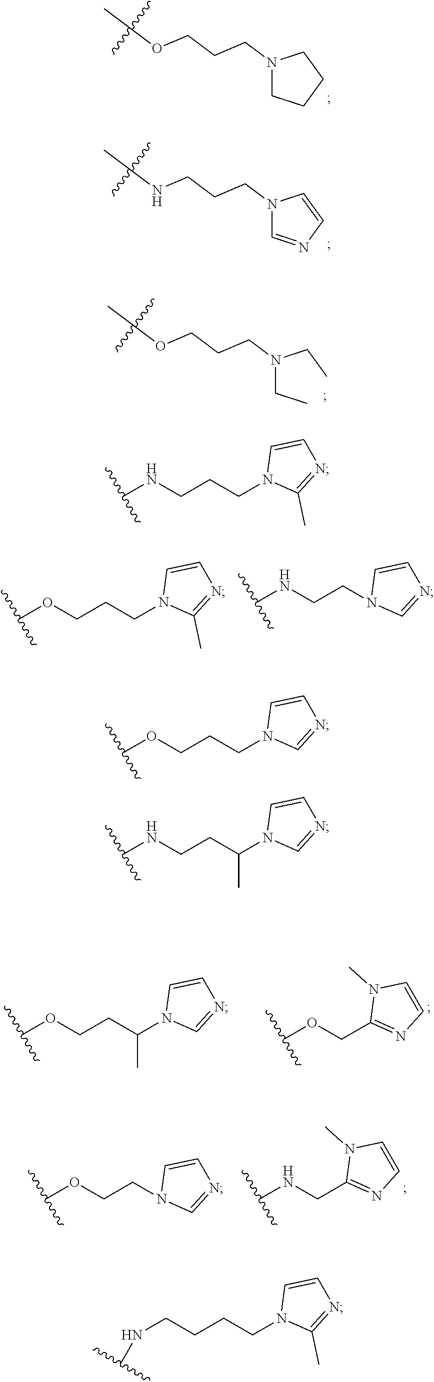

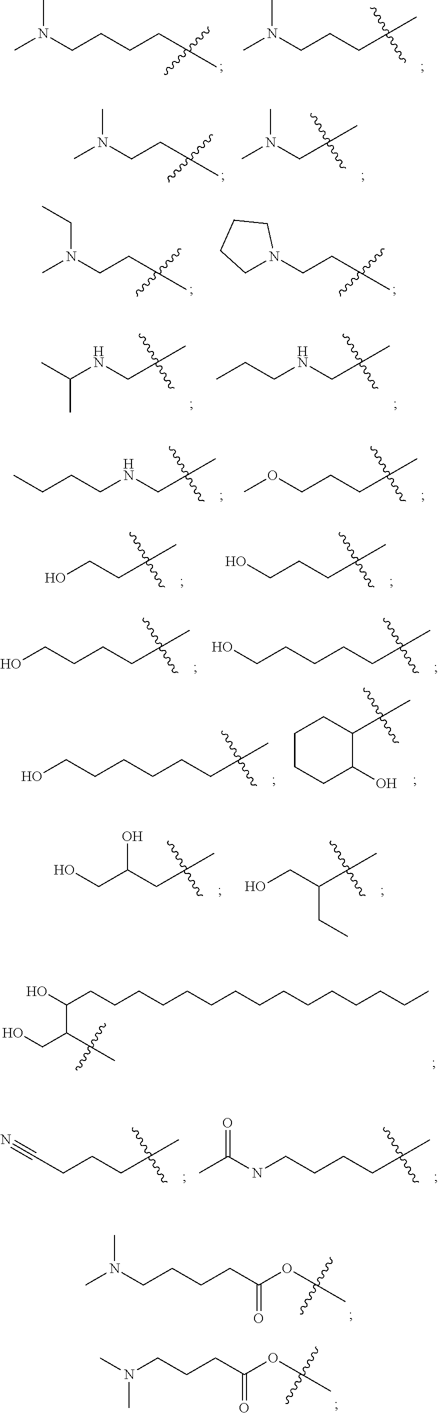

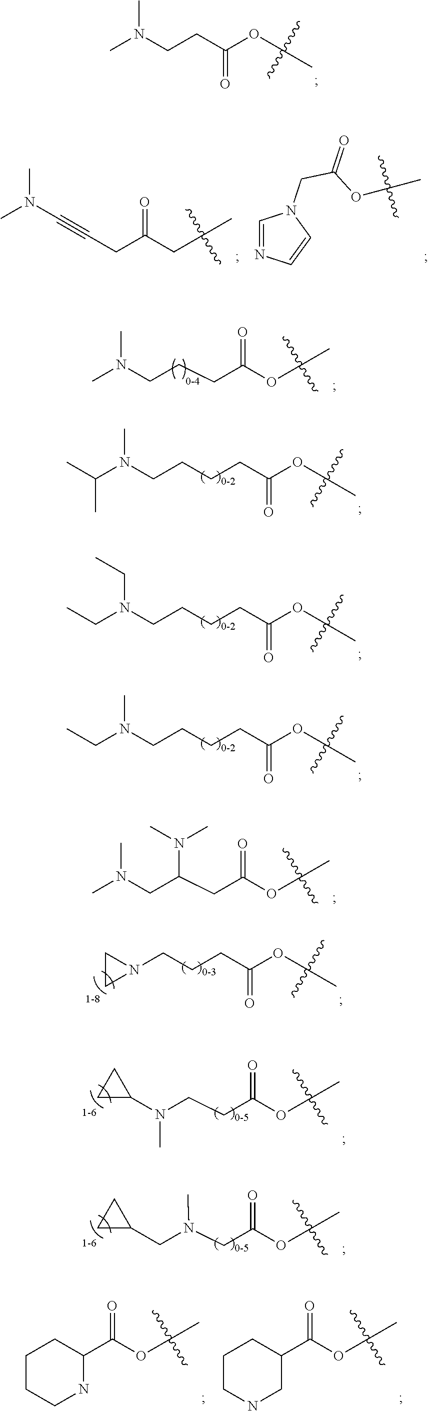

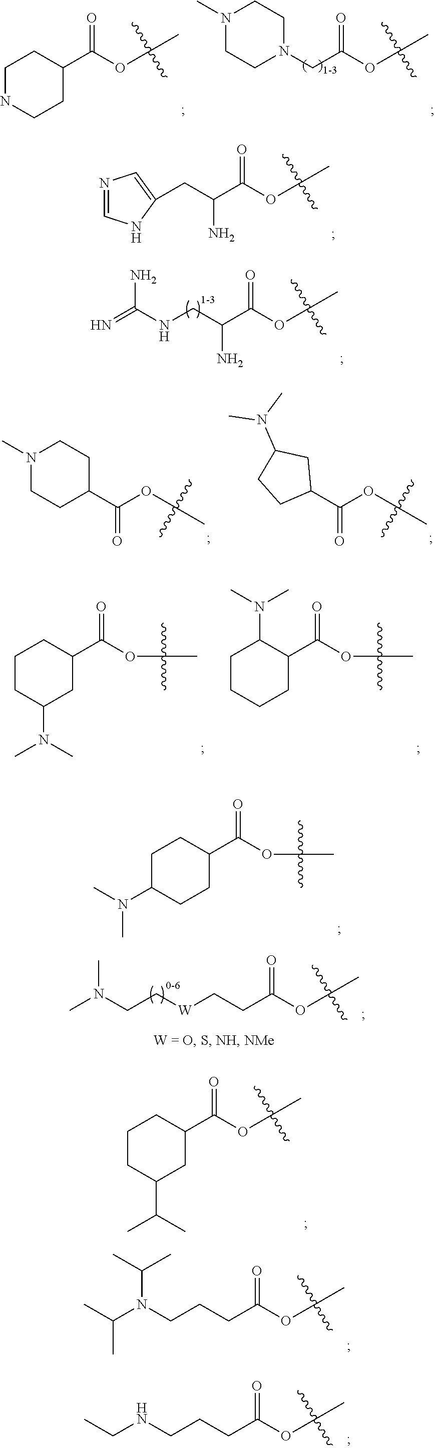

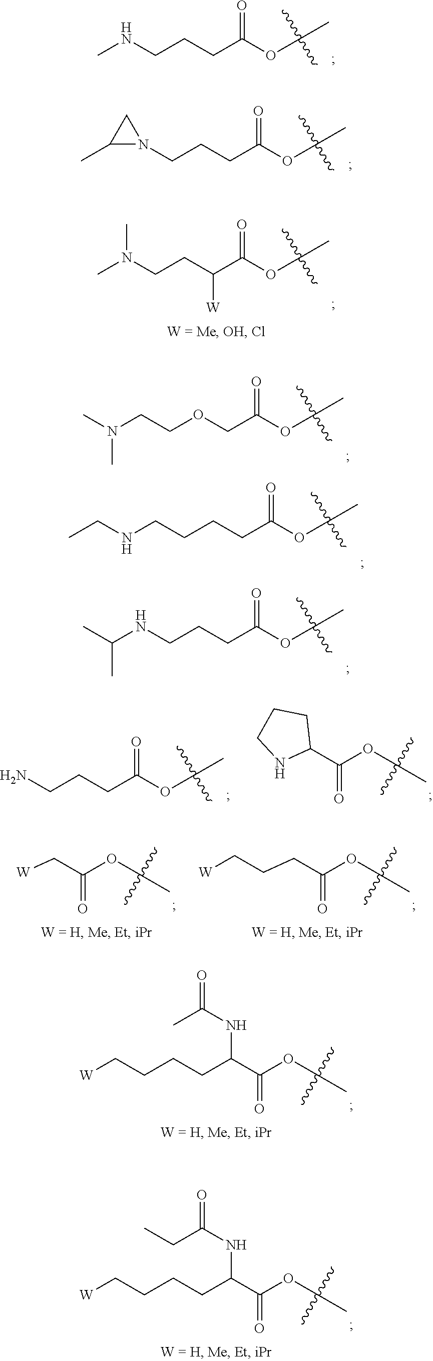

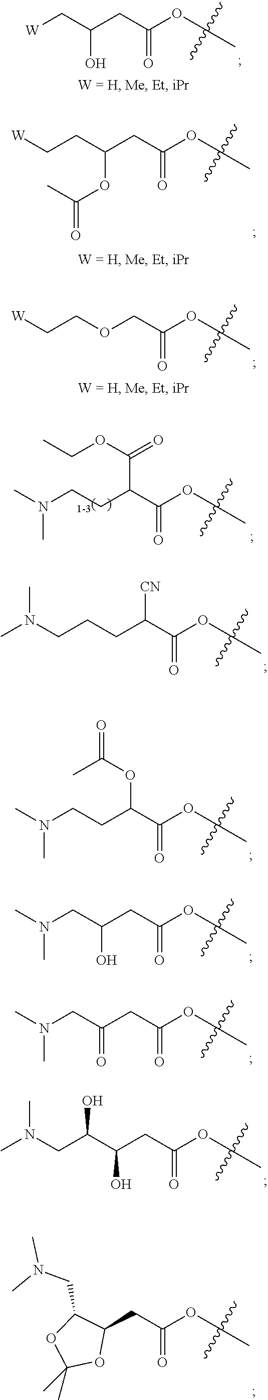

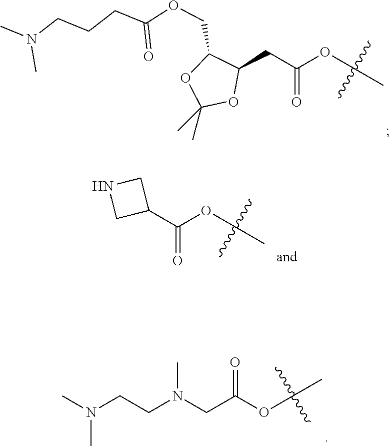 [SMILES: *OC(=O)C[C@H]1OC(C)(C)O[C@@H]1COC(=O)CCCN(C)C ; *OC(=O)C1CNC1 ; *OC(=O)CN(C)CCN(C)C]

In some embodiments, the ionizable lipid is represented by:

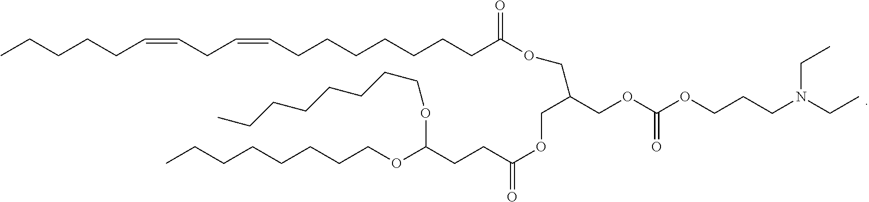 [SMILES: CCCCC/C=C\C/C=C\CCCCCCCC(=O)OCC(COC(=O)CCC(OCCCCCCCC)OCCCCCCCC)COC(=O)OCCCN(CC)CC]

In some embodiments, the circular RNA polynucleotide comprises a core functional element comprising, in the following order: (i) a translation initiation element (TIE), and (ii) a coding element encoding for the muscle protein or protein complex.

In some embodiments, the muscle protein or protein complex is a human or humanized muscle protein or protein complex. In some embodiments, the muscle protein or protein complex is a smooth, skeletal, or cardiac muscle protein or protein complex. In some embodiments, the muscle protein or protein complex is a smooth muscle myosin, actin, tropomyosin, calponin, or caldesmon, or a fragment, isoform, or variant thereof. In some embodiments, the muscle protein or protein complex is a skeletal or muscular sarcolemmal protein, laminin, dystroglycan, collagen, actin, myosin, myofibrillar protein, dystrophin, or intermediate protein, or a fragment, isoform, or variant thereof.

In some embodiments, the muscle protein or protein complex is dystrophin or a fragment, isoform, or variant thereof. In some embodiments, the dystrophin variant comprises a mutation, truncation, deletion, or insertion of a naturally occurring or synthetic dystrophin. In some embodiments, the dystrophin variant is at least about 1900 amino acids in length. In some embodiments, the dystrophin variant comprises a dystrophin central rod domain in whole or in part from a human full-length dystrophin. In some embodiments, the dystrophin variant lacks a C-terminal domain. In some embodiments, the dystrophin variant comprises about 6-24 spectrin-like repeats. In some embodiments, the dystrophin variant further comprises a synthrophin-binding domain. In some embodiments, the dystrophin variant comprises a hinge domain. In some embodiments, the dystrophin variant comprises a hinge 1, hinge 2, hinge 3 or hinge 4 domain. In some embodiments, the dystrophin variant comprises a Becker variant. In some embodiments, the dystrophin or fragment, isoform, or variant thereof comprises a full-length naturally occurring or synthetic dystrophin. In some embodiments, the dystrophin or fragment, isoform, or variant thereof comprises at least 3500 amino acids in length. In some embodiments, the dystrophin or fragment, isoform, or variant thereof comprises or consists of a sequence selected from Table 1.

In some embodiments, the muscle protein or protein complex is a dystrophin-associated protein or a fragment thereof. In some embodiments, the dystrophin-associated protein comprises a α-dystroglycan, β-dystroglycan, sarcoglycans, sarcospan, dystrobrevin, syntrophins, neuronal nitric oxide synthase or fragment thereof.

In some embodiments, the coding element comprises an intein-containing split protein gene. In some embodiments, the intein-containing split protein gene comprises a split dystrophin gene. In some embodiments, the TIE comprises an untranslated region (UTR) or a fragment thereof, an aptamer complex or a fragment thereof, or a combination thereof.

In some embodiments, the UTR or fragment thereof is derived from a viral or eukaryotic messenger RNA. In some embodiments, the UTR or fragment thereof comprises a viral or eukaryotic messenger RNA. In some embodiments, the UTR or fragment thereof comprises a viral internal ribosome entry site (IRES) or eukaryotic IRES. In some embodiments, the IRES comprises one or more modified nucleotides compared to the wild-type viral IRES or eukaryotic IRES.

In some embodiments, the aptamer complex or fragment thereof comprises a natural or synthetic aptamer sequence. In some embodiments, the aptamer complex or a fragment thereof comprises more than one aptamer.

In some embodiments, the TIE comprises a UTR and an aptamer complex. In some embodiments, the UTR is located upstream to the aptamer complex.

In some embodiments, the TIE comprises an accessory element. In some embodiments, the accessory element comprises a miRNA binding site or a fragment thereof, a restriction site or a fragment thereof, an RNA editing motif or a fragment thereof, a zip code element or a fragment thereof, an RNA trafficking element or a fragment thereof, or a combination thereof. In some embodiments, the accessory element comprises a binding domain to an IRES transacting factor (ITAF). In some embodiments, binding domain comprises a polyA region, a polyC region, a poly

AC region, a polyprimidine tract, or a combination or variant thereof. In some embodiments, the ITAF comprises a poly (rC)-binding protein 1 (PCBP1), PCBP2, PCBP3, PCBP4, poly (A)-binding protein 1 (PABP1), polyprimidine-tract binding protein (PTB), Argonaute protein family member, HNRNPK (heterogeneous nuclear ribonucleoprotein K protein), or La protein, or a fragment or combination thereof.

In some embodiments, the core functional element comprises a termination element.

In some embodiments, a provided pharmaceutical composition has an in vivo duration of therapeutic effect in humans of at least 20 hours. In some embodiments, a provided pharmaceutical composition has functional half-life of at least 6 hours.

In some embodiments, the circular RNA polynucleotide comprises a spacer sequence. In some embodiments, the circular RNA polynucleotide comprises two or more duplex forming sequences. In some embodiments, the circular RNA polynucleotide comprises an exon element. In some embodiments, the circular RNA polynucleotide comprises a 5′ exon element and a 3′ exon element.

In some embodiments, the transfer vehicle comprises a nanoparticle. In some embodiments, the nanoparticle is a lipid nanoparticle, a core-shell nanoparticle, or a biodegradable nanoparticle. In some embodiments, the nanoparticle comprises one or more cationic lipids, ionizable lipids, or poly β-amino esters. In some embodiments, the nanoparticle comprises a DSPE, DOPE, or a combination thereof. In some embodiments, the nanoparticle comprises one or more non-cationic lipids. In some embodiments, the nanoparticle comprises one or more PEG-modified lipids, polyglutamic acid lipids, or hyaluronic acid lipids. In some embodiments, the nanoparticle comprises cholesterol. In some embodiments, the nanoparticle comprises arachidonic acid, leukotriene, or oleic acid.

In another aspect, provided herein are precursor RNA polynucleotides of circular RNA polynucleotides provided herein.

In some embodiments, the precursor RNA polynucleotide comprises two or more expression sequences encoding for the muscle protein or protein complex. In some embodiments, the precursor RNA polynucleotide comprises a polynucleotide sequence encoding a proteolytic cleavage site or a ribosomal stuttering element between the first and second expression sequence. In some embodiments, the ribosomal stuttering element is a self-cleaving spacer. In some embodiments, the precursor RNA polynucleotide comprises a polynucleotide sequence encoding 2A ribosomal stuttering peptide.

In some embodiments, the precursor RNA polynucleotide comprises two or more internal ribosome entry sites (IRESs).

In some embodiments, the precursor RNA polynucleotide comprises a first TIE, a first coding element, a first termination sequence, optionally a spacer, a second TIE, a second coding element, and second a termination sequence, wherein the first TIE and the second TIE each comprises an IRES

In some embodiments, the precursor RNA polynucleotide comprises an intron element. In some embodiments, the precursor RNA comprises a 5′ intron element and a 3′ intron element. In some embodiments, the precursor RNA comprises a spacer sequence. In some embodiments, the precursor RNA comprises an affinity sequence or a leading untranslated sequence. In some embodiments, the precursor RNA comprises a 5′ duplex sequence and a 3′ duplex sequence.

In some embodiments, the precursor RNA is transcribed from a vector or DNA comprising a PCR product, a linearized plasmid, non-linearized plasmid, linearized minicircle, a non-linearized minicircle, viral vector, cosmid, ceDNA, or an artificial chromosome.

In another aspect, provided herein are methods of producing muscle protein or protein complex in a muscle cell or muscle tissue, comprising delivering a provided pharmaceutical composition using systemic administration. In some embodiments, the systemic administration comprises intravenous (i.v.) injection. In some embodiments, the muscle tissue is cardiac or diaphragm muscle tissue.

In another aspect, provided herein are methods of treating a subject in need thereof comprising administering a therapeutically effective amount of a provided pharmaceutical composition. In some embodiments, the subject has muscular dystrophy, dystroglycanopathy, collagen VI myopathy, Limb-girdle muscular dystrophies (LGMD), myofibrillar myopathy, or dilated cardiomyopathy. In some embodiments, the subject has Becker's disease, Duchenne Muscular Dystrophy (DMD), or Congenital Muscular Dystrophy type 1A (MDCIA), Ullrich congenital muscular dystrophy (UCMD), Walker-Warburg syndrome, Muscle-eye-brain syndrome or Bethlem myopathy (BM). In some embodiments, the therapeutically effective amount is less than or equal to about 3 mg/kg of circular RNA polynucleotide. In some embodiments, the therapeutically effective amount is less than or equal to about 1 mg/kg of circular RNA polynucleotide.

### BRIEF DESCRIPTION OF DRAWINGS

FIGS. 1A and 1B depict protein expression in C2C12 mouse myoblasts following transfection of circular RNA encoding firefly luciferase in a lipofectamine 3000 solution. FIG. 1A is an image of the myoblasts 24 hours following transfection of the circular RNA construct for the 96 wp with 3 cell densities of 3000, 10000, and 15000 cells. FIG. 1B provides the relative light units produced by the cells for the 3 cell densities each at 0 to 200 ng dosage of the circular RNA construct.

FIG. 2 illustrates protein expression in human skeletal muscle myoblasts from three different donors following transfection of circular RNA encoding firefly luciferase in a lipofectamine 3000 solution. Cells were dosed with either 0, 10, 25, 50, 100, or 200 ng of circular RNA and analyzed for relative light units (RLU) produced.

FIGS. 3A and 3B show protein expression in human skeletal muscle myotubes from two different donors following transfection of circular RNA encoding firefly luciferase in a lipofectamine solution at various circular RNA dosages. FIG. 3A provides an image of the myotubes before, on, and after the date of transfection of the circular RNA construct to the cells.

FIG. 3B provides the RLU produced by the human skeletal muscle myotubes from two different donor cells, after 24 hours post transfection of the circular RNA constructs.

FIG. 4 illustrates micro-dystrophin expression in primary human skeletal muscle myotubes following transfection of circular RNA encoding micro-dystrophin using LNP comprising Lipid 1 (LP1) at varying dosages. In B and C sets of lanes, neutral and acidic ethyl lauryl arginate (ELA) with LNP are formulated respectively at 4.50B to optimize expression. A set of lanes comprises a 4.5B of the Lipid 1 LNP. Circular RNA encoding Green Fluorescent Protein (GFP) and transfected by the same lipid is used as a control.

FIG. 5 provides a Western blot following a HeLa Cell-Free translation (CFT) assay of full-length dystrophin (427 kD) and micro-dystrophin (167 kD) following transfection of circular RNA encoding said proteins at 500 ng or 1000 ng dosages.

FIG. 6 shows in vitro mini-dystrophin protein expression in human skeletal muscle myotubes following transfection at varying dosages of a circular RNA-LNP construct comprising either Lipid 1 (LP1) or a comparator lipid.

FIG. 7 illustrates in vivo expression in mdx of micro-dystrophin following intramuscular administration of circular RNA encoding said protein and encapsulated within a Lipid 1 (LP1) or comparator lipid to quadricep muscle. The protein was visualized by immunoprecipitation assay and detected using both a V5 and dystrophin specific antibody.

FIG. 8 depicts an immunoprecipitation assay of the quadriceps muscle containing expression of micro-dystrophin following intravenous administration of circular RNAs encoding said protein and encapsulated in a LNP comprising Lipid 1 (LP1) in mdx mice.

FIGS. 9A and 9B provides an immunoprecipitation assay of dystrophin produced following transfection of circular RNA encoding micro-dystrophin at varying dosages. FIG. 9A illustrates an immunofluorescence assay of a cross-section of a tibialis anterior muscle stained with anti-dystrophin antibody. The white regions of the figure show localization of the micro-dystrophin within the transfected cells. FIG. 9B provides the immunoprecipitation analysis of the dystrophin proteins formed following intravenous administration at varying dosages to the cells using said circular RNA construct.

FIG. 10 depicts exemplary dystrophin variants schematics, including micro-dystrophins compared to a full-length dystrophin.

FIGS. 11A and 11B provide a liver Western blot (FIG. 11A) and a liver immunoprecipitation analysis (FIG. 11B) of protein produced following multiple dosing methods of a circular RNA encoding micro-dystrophin and encapsulated in Lipid 1 (LP1).

FIG. 12 shows protein expression of full-length dystrophin (427 kD) in human skeletal muscle myotubes following transfection of Lipofectamine 3000 (Lipo) or Messenger Max (Mmax) (FIG. 12A) or Lipid 1 (LP1) or a comparator lipid (CP) (FIG. 12B) at varying dosages.

FIGS. 13A, 13B, and 13C illustrate in vivo expression of firefly luciferase in various organ tissues (i.e., liver, spleen, kidney, lungs, heart, diaphragm, quadriceps, and calf) following dosing circular RNA encoding said protein and encapsulated within a LNP comprising Lipid 1 (LP1). FIG. 13A provides total flux of the firefly luciferase in each organ tissue. FIG. 13B provides flux distribution in the form of percent protein expression. FIG. 13C illustrates ex vivo imaging of the liver, spleen, kidney, lungs, heart, diaphragm, quadriceps, and calf of the mouse 6 hours following transfection.

FIG. 14 is an image of a western blot of V5 tag specific antibody of HeLa Cell-Free Translation Assay samples. Micro dystrophin is present at 165 kDa (most left band), the Becker variant is present at 228 kDa (middle band) and the full-length dystrophin is present at 427 kDa (most right band).

FIG. 15 is an image of a western blot of V5 tag specific antibody of myotube cell lysates of myotubes incubated with lipid nanoparticles comprising Lipid 1 and circular RNA designed to encode Becker variant dystrophin (containing about 46% internal deletion of a full-length dystrophin), micro dystrophin, or vinculin (positive control).

FIG. 16 is an image of a western blot of V5 tag specific antibody of myotube cell lysates of myotubes transfected with circular RNA designed to encode full length dystrophin.

FIG. 17A is an image of a western blot depicting expression of micro-dystrophin in gastrocnemius muscles tissue from mdx mouse muscle following intravenous injection LNPs formulated with oRNA encoding micro-dystrophin.

FIG. 17B are images of sections of quadriceps muscle of mdx mice following intravenous injection with LNPs formulated with oRNA encoding micro-dystrophin. The top panels have laminin-211 outlining the sarcolemma of the myofibers overlayed with micro-dystrophin with DAPI stain showing individual nuclei. Bottom panels of the same figure are replicates of the top without the laminin-211 outline to give a view of micro-dystrophin expression at the myofiber with DAPI outlining nuclei.

FIG. 18 is a diagram depicting domains of Dystrophin variant proteins.

FIG. 19 depicts an immunoprecipitation/western blot assay of the diaphragm muscle containing expression of Becker variant or micro-dystrophin following intravenous administration of circular RNAs encoding said proteins and encapsulated in a LNP comprising Lipid 1 (LP1) in mdx mice.

### DETAILED DESCRIPTION

In various embodiments, provided herein are lipid nanoparticle (LNP) compositions and methods for systemically delivering nucleic acid constructs to muscle tissue for making muscle proteins, muscle protein complexes, and/or fragments thereof. In some embodiments, the present invention comprises an LNP formulation comprising a circular RNA or linear RNA polynucleotide encoding one or more muscle protein, protein complex, or fragment thereof.

### Definitions

As used herein, the terms “circRNA,” “circular polyribonucleotide,” “circular RNA,”

“ORNA,” and “circular RNA polynucleotide” are used interchangeably and refer to a polyribonucleotide that forms a circular structure through covalent bonds.

As used herein, the term “biologically active” used in connection with a molecule, such as a biological molecule (e.g., a nucleic acid, a peptide, a polypeptide, or a protein), refers to that the molecule is capable of exerting one or more biological effects (e.g., altering the physical or chemical properties or functions of other substances) and/or has activity in a biological system (e.g., a cell, a tissue, or an organism).

As used herein, the term “core functional element” refers to a portion of a circular RNA containing at least one “expression sequence,” which, as used herein, refers to a nucleic acid sequence that encodes any one or more desired peptides, polypeptides, or proteins. In some embodiments, the core functional element contains nucleic acid sequences useful for enhanced expression of such protein. In some embodiments, the expression sequence comprises a nucleic acid sequence encoding for a biologically active peptide, polypeptide, or protein, which nucleic acid sequence is referred to as a “coding element” or “coding region.” In some embodiments, the expression sequence comprises a nucleic acid sequence having regulatory functions, including but not limited to allowing the peptide(s), polypeptide(s), or protein(s) expressed by the expression sequence to act as a biomarker or adjuvant to a specific cell, which nucleic acid sequence is referred to as a “noncoding element,” “regulatory nucleic acid”, or “non-coding nucleic acid.” In some embodiments, an exemplary expression sequence comprises a plurality of nucleotide triads, each of which can code for an amino acid and is termed as a “codon.”

As used herein, the term “DNA template” refers to a DNA sequence capable of being transcribed into a linear RNA polynucleotide.

As used herein, “transfer vehicle” includes any of the standard pharmaceutical carriers, diluents, excipients, and the like, which are generally intended for use in connection with the administration of biologically active agents, including nucleic acids. In certain embodiments of the present invention, the transfer vehicles (e.g., lipid nanoparticles) are prepared to encapsulate one or more materials or therapeutic agents (e.g., circRNA). The process of incorporating a desired therapeutic agent (e.g., circRNA) into a transfer vehicle is referred to herein as or “loading” or “encapsulating.” The transfer vehicle-loaded or-encapsulated materials (e.g., circRNA) may be completely or partially located in the interior space of the transfer vehicle, within a bilayer membrane of the transfer vehicle, or associated with the exterior surface of the transfer vehicle.

As used herein, an “translation initiation element” or “TIE” refers to an RNA sequence or structural element capable of initiating translation of a polypeptide in the absence of a typical RNA cap structure. In some embodiments, TIE is about 10-nucleotide (nt) to about 1000 nt in length or longer. In some embodiments, TIE is about 500 nt to about 700 nt in length. In certain embodiments, an “internal ribosome entry site” or “IRES” derived from viral IRES sequences can be used as a TIE.

As used herein, the term “muscle protein or protein complex” refers to (1) a protein or protein complex naturally expressed in normal human muscle tissue, (2) a protein or protein complex not naturally expressed in normal human tissue but has a desired effect, such as a therapeutic effect, if expressed in muscle tissue, or (3) variants of a protein or protein complex of (1) or (2).

As used herein, the term “therapeutic protein” refers to any protein that, when administered to a subject directly or indirectly in the form of a translated nucleic acid, has a therapeutic, diagnostic, and/or prophylactic effect and/or elicits a desired biological and/or pharmacological effect.

### Circular RNA

The present invention provides, among other things, compositions and methods for making and using circular RNA polynucleotides capable of encoding muscle proteins. Circular RNA polynucleotides lack the free ends necessary for exonuclease-mediated degradation, causing them to be resistant to several mechanisms of RNA degradation and granting extended half-lives when compared to an equivalent linear RNA. Circularization may allow for the stabilization of RNA polynucleotides that generally suffer from short half-lives and may improve the overall efficacy of exogenous mRNA in a variety of applications. In some embodiments, the functional half-life of the circular RNA polynucleotides provided herein in eukaryotic cells (e.g., mammalian cells, such as human cells) as assessed by protein synthesis is at least 20 hours (e.g., at least 80 hours). As described herein, the inventive circular RNA comprises various elements used to aid circularization of a precursor linear RNA polynucleotide and/or expression of a protein (e.g., core functional element, exon segments, intron segments, spacers, duplex regions, and accessory elements).

Core functional element

In some embodiments, the circular RNA polynucleotide comprises a core functional element. The core functional element is designed to have the essential elements for protein translation including a translation initiation element (TIE), an expression sequence comprising one or more coding or noncoding elements, and optionally a termination sequence (e.g., a stop codon or a stop cassette). In some embodiments, the core functional element comprises a TIE operably connected to the expression sequence. In some embodiments, the expression sequence encodes for one or more muscle proteins, muscle complexes, or fragments thereof. In certain embodiments, the core functional element comprises one or more spacer sequences.

### Translation Initiation Elements (TIEs)

As provided herein, TIEs are capable of allowing translation efficiency of an encoded protein. In some embodiments, a provided core functional element comprises no TIEs. In some embodiment, the core functional element comprises no coding elements and no TIEs. In some embodiments, a provided core functional element comprises at least one TIE. In some embodiments, the core functional element comprises one or more coding elements and one or more TIEs.

In some embodiments, a TIE comprises an untranslated region (UTR). In certain embodiments, the TIE provided herein comprise an internal ribosome entry site (IRES). Inclusion of an IRES permits the translation of one or more open reading frames from a circular RNA (e.g., open reading frames that form the expression sequences). The IRES element attracts a eukaryotic ribosomal translation initiation complex and promotes translation initiation. See, e.g., Kaufman et al., Nuc. Acids Res. (1991) 19:4485-4490; Gurtu et al., Biochem. Biophys. Res. Comm. (1996) 229:295-298; Rees et al., BioTechniques (1996) 20:102-110; Kobayashi et al., BioTechniques (1996) 21:399-402; and Mosser et al., BioTechniques 1997 22 150-161.

A multitude of IRES sequences are available and include sequences derived from a wide variety of viruses, such as from leader sequences of picornaviruses such as the encephalomyocarditis virus (EMCV) UTR (Jang et al., J. Virol. (1989) 63:1651-1660), the polio leader sequence, the hepatitis A virus leader, the hepatitis C virus IRES, human rhinovirus type 2 IRES (Dobrikova et al., Proc. Natl. Acad. Sci. (2003) 100 (25): 15125-15130), an IRES element from the foot and mouth disease virus (Ramesh et al., Nucl. Acid Res. (1996) 24:2697-2700), a giardiavirus IRES (Garlapati et al., J. Biol. Chem. (2004) 279 (5): 3389-3397), and the like.

In certain embodiments, the IRES comprises in whole or in part from a eukaryotic or cellular IRES. In some embodiments, the IRES is from a human gene.

Exemplary IRES sequences are listed at the World Wide Web at iresite.org. Further, examples of IRES sequences can be used with circular RNA constructs are described in PCT applications PCT/US2019/035531, PCT/US2020/034418, PCT/US2020/063494, PCT/US2016/036045 and PCT/US2021/023540, the contents of each of which are incorporated here by reference in their entireties.

Production for circular RNA polynucleotides

There are various methods known in the art for forming a circular RNA polynucleotide. The biogenesis of an engineered circular RNA may be from a precursor RNA. In certain embodiments, this precursor RNA is a linear or circular RNA polynucleotide with shared essential elements (e.g., expression sequences, IRES, and exon elements). For the circular RNAs formed using a linear precursor nucleotide (i.e., a linear RNA precursor), the 5′ and 3′ ends of the linear precursor nucleotide need to be joined. Orientation of the two ends can be aided using a linear or hairpin helper nucleotide or linear splint ligation. The methods of allowing circularization may include enzymatic ligation (e.g., T4 DNA ligase, T4 RNA ligase 1, and T4 RNA ligase 2), chemical ligation (e.g., natural phosphodiester linkages or non-natural linkages, such as using oxime circularization or click circularization), ribozyme circularization (Müller & Appel, RNA Biol. 2017; 14 (8): 1018-1027), ribozyme terminal cleavage followed by enzymatic ligation (Litke & Jaffrey, Nature Biotechnology, 2019), or use of a viroid, satellite virus, hepatitis delta virus (HDV) or HDV-like ribozyme (Pena, Ceprian, and Cervera, Cells, 2020).

Two common techniques used in the art for ribozyme circularization are canonical splicing (i.e., lariat model) or non-canonical splicing (i.e., backsplicing model). Circular RNAs generated by either method may be transcribed from the precursor RNA in vitro or in vivo as described in the art. Circularization may be conducted using an autocatalytic/self-splicing intron.

In certain embodiments, the self-splicing intron may comprise or consist essentially of a natural or synthetic introns. In some embodiments, the self-splicing intron is a group I or group II intron. Intron fragments or intron segments or intron elements, as known in the art, may include exon sequences in a permuted intron-exon (PIE) splicing format. This PIE splicing technique is described in PCT/US2019/035531, PCT/US2020/034418, and PCT/US2020/063494; each of these applications are incorporated by reference in their entireties. Other circularization methods are described in PCT/US2018/027665, PCT/US2016/036045, and PCT/US2014/037795.

As contemplated herein, the precursor RNA capable of forming the circular RNA may be generated using a vector or a DNA template. In certain embodiments the vector or DNA template may comprise a PCR product, linearized plasmid, non-linearized plasmid, linearized minicircle, a non-linearized minicircle, viral vector, cosmid, ceDNA, artificial chromosome or another nucleotide capable of translating an RNA polynucleotide available in the art.

Lipid nanoparticles

Disclosed herein are various embodiments of LNP formulations for nucleic acid cargoes. Such LNP may contain (i) an ionizable lipid for encapsulation and for endosomal escape, (ii) a neutral lipid for stabilization, (iii) a helper lipid, also for stabilization, and (iv) a stealth lipid. By “lipid nanoparticle” is meant a particle that comprises a plurality of (i.e. more than one) lipid molecules physically associated with each other by intermolecular forces. The LNPs may be, e.g., microspheres (including unilamellar and multilamellar vesicles, e.g., “liposomes”-lamellar phase lipid bilayers that, in some embodiments, are substantially spherical-and, in more particular embodiments, can comprise an aqueous core, e.g., comprising a substantial portion of RNA molecules), a dispersed phase in an emulsion, micelles, or an internal phase in a suspension.

The LNP compositions provided herein are capable of being taken up by muscle cells. In some embodiments, such LNP compositions uptake does not require targeting moieties of the muscle cells (e.g., targeting cell surface receptors of muscle cells). In some embodiments, the muscle cells may be in skeletal muscle or other muscle tissue such as cardiac, diaphragm.

In some embodiments, LNP compositions containing ionizable lipids and circular RNAs described herein may be used to express muscle proteins in muscle tissue via systemic administration. The described methods of delivering and expressing muscle proteins using such LNP formulations may be therapeutically useful without the need for added targeting moieties (e.g., of the muscles cells). In some embodiments, the described LNP compositions are biodegradable, in that they do not accumulate to cytotoxic levels in vivo when administered at a therapeutically effective dose. In some embodiments, the LNP compositions do not cause an innate immune response that leads to substantial adverse effects when administered at a therapeutically effective dose level. In some embodiments, the LNP compositions provided herein do not cause toxicity when administered at a therapeutically effective dose level.

### Ionizable Lipids

In some embodiments, an ionizable lipid of the present disclosure comprises or consists of a lipid of Formula (1):

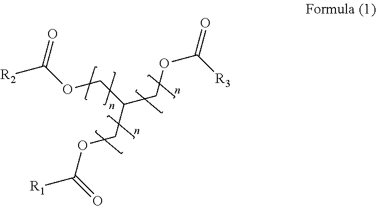

wherein:
each n is independently an integer from 1-15;
R₁ and R₂ are each independently selected from a group consisting of:

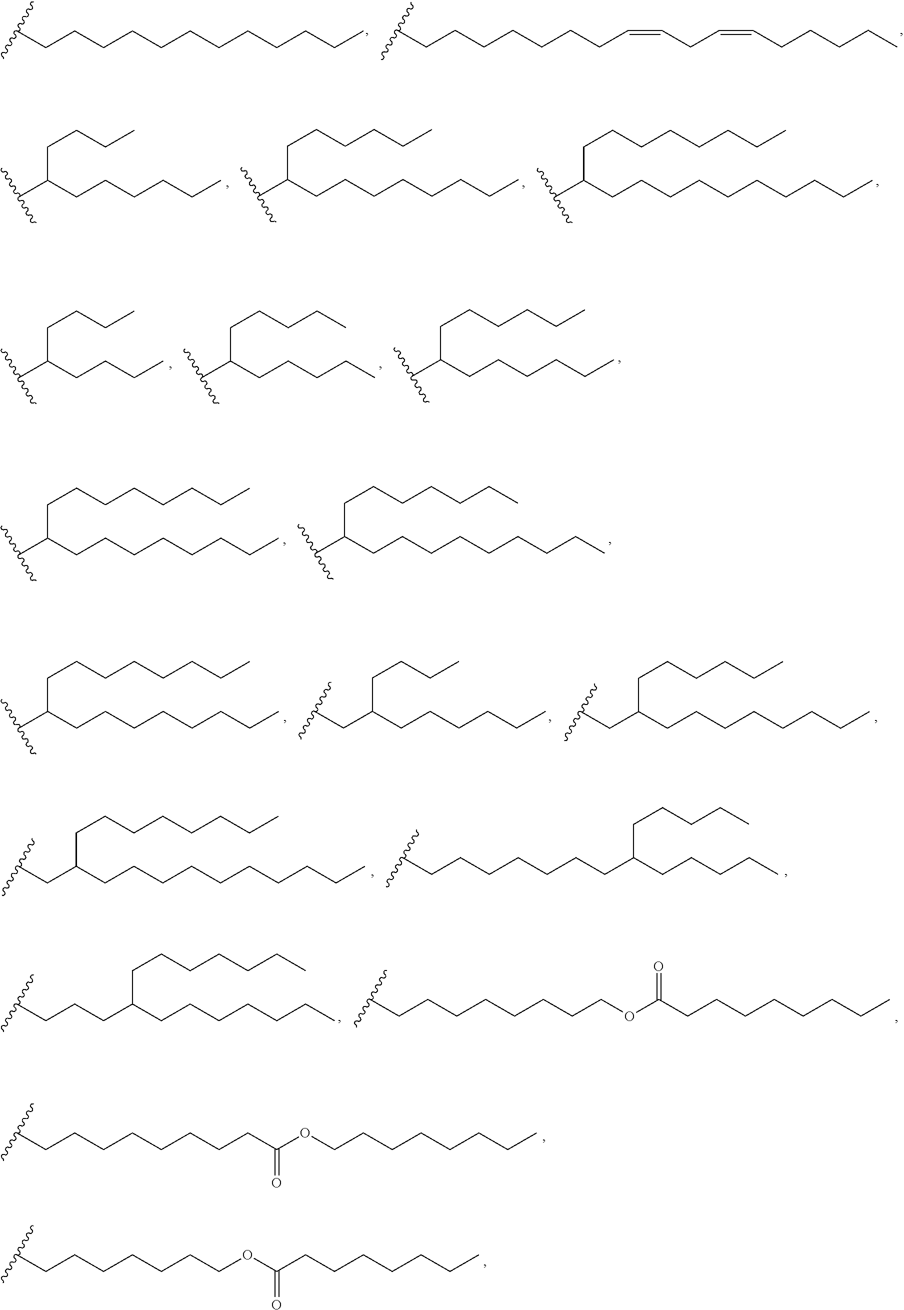

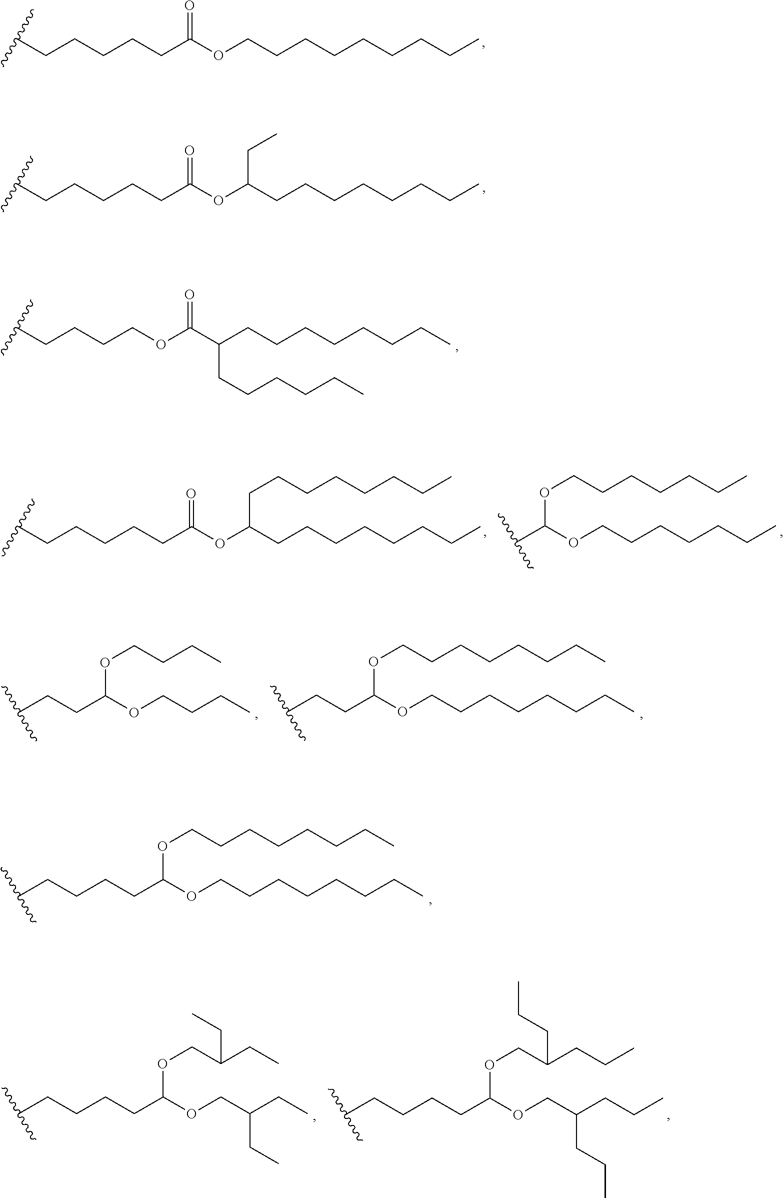

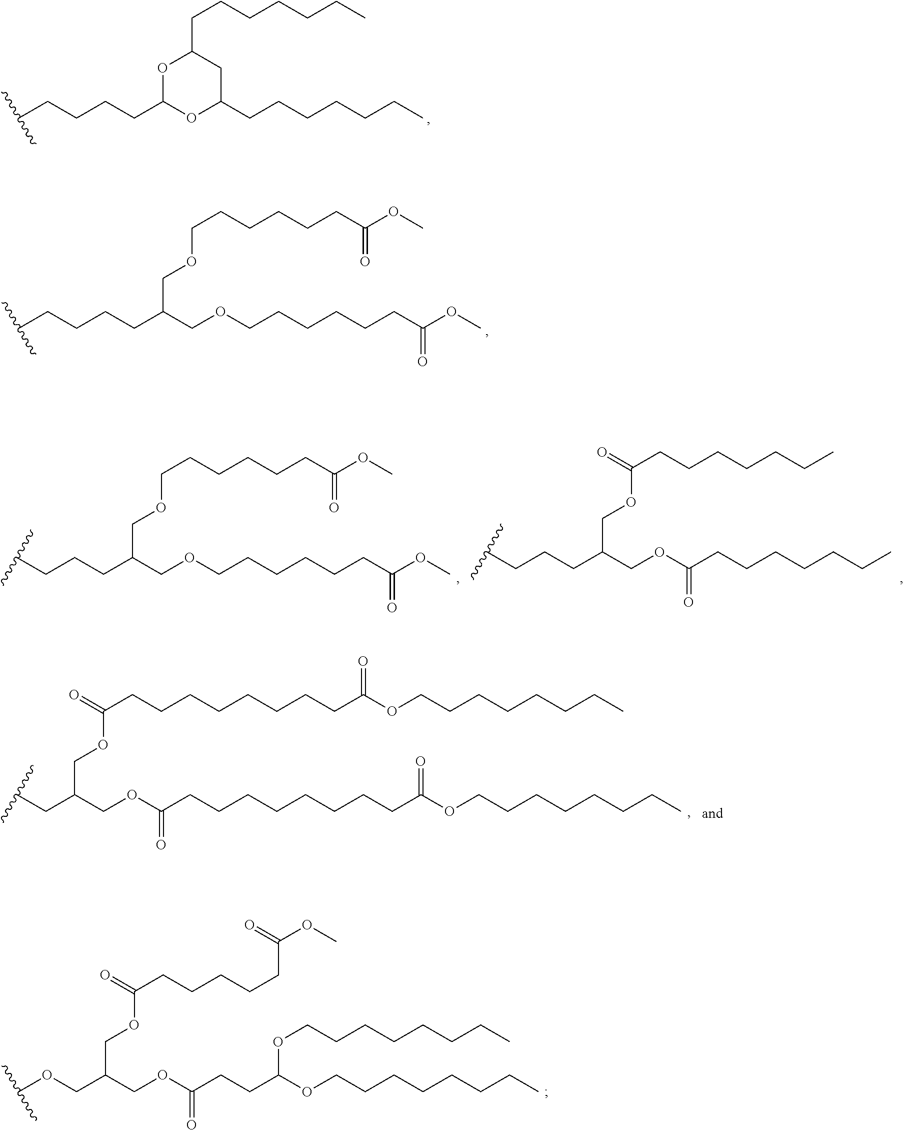

and
R₃ is selected from a group consisting of:

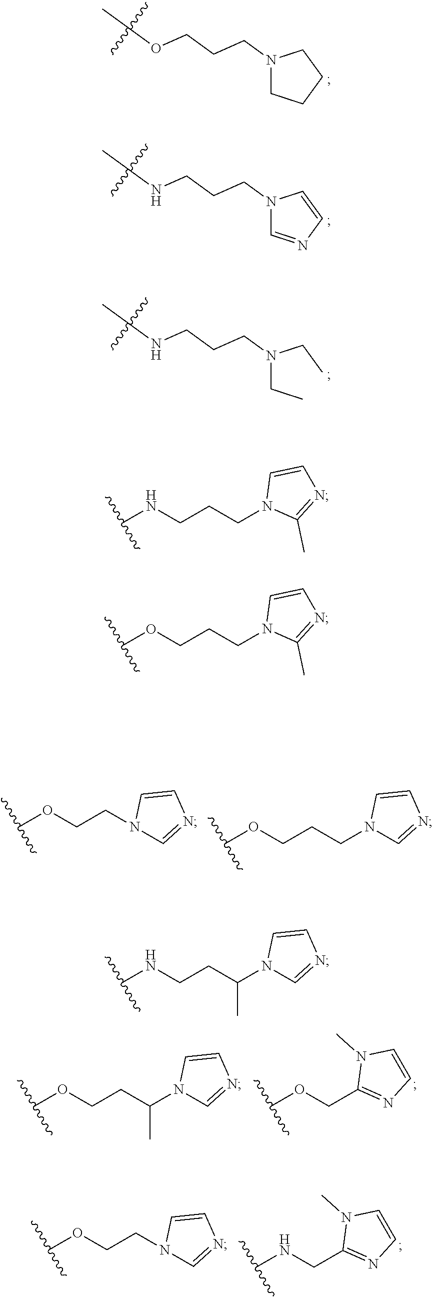

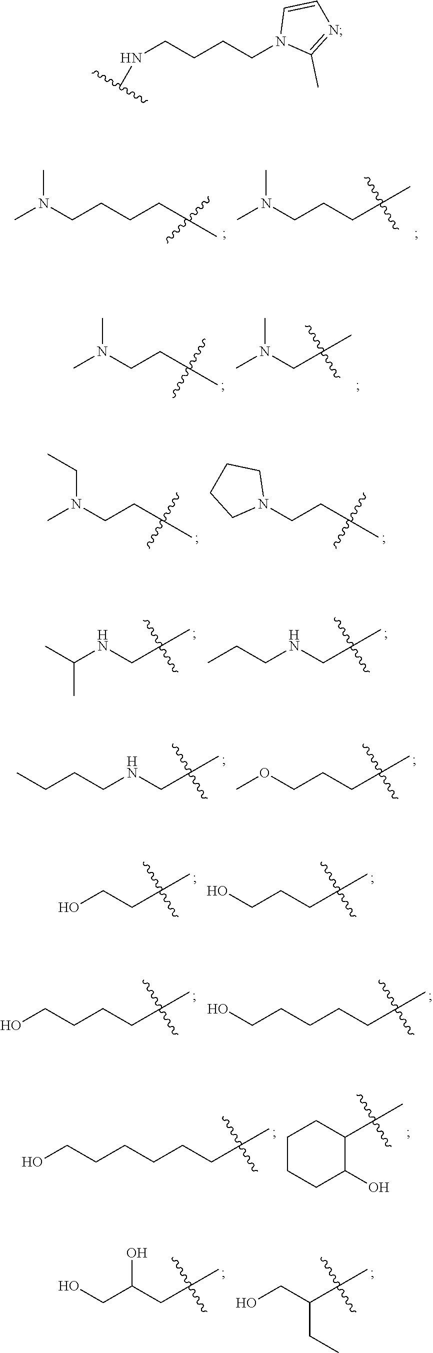

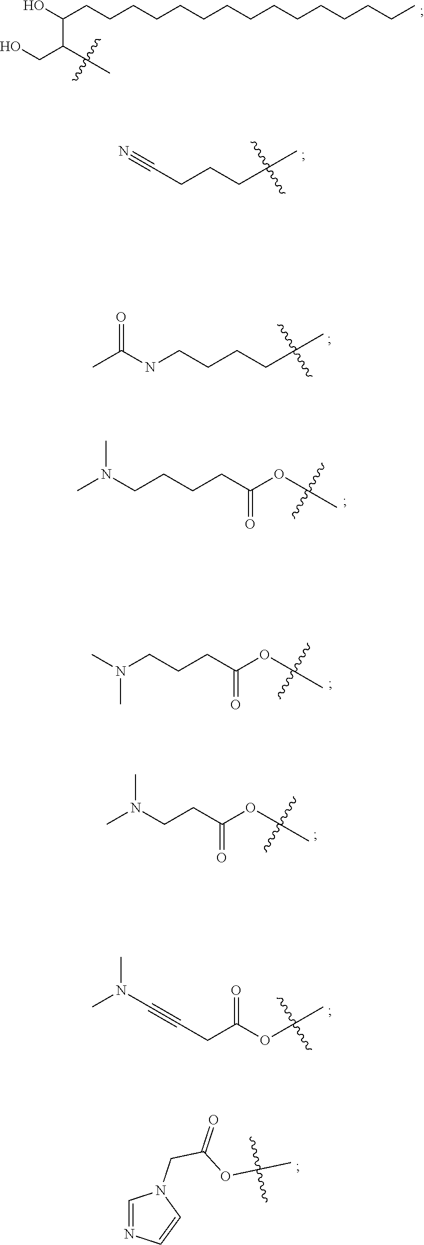

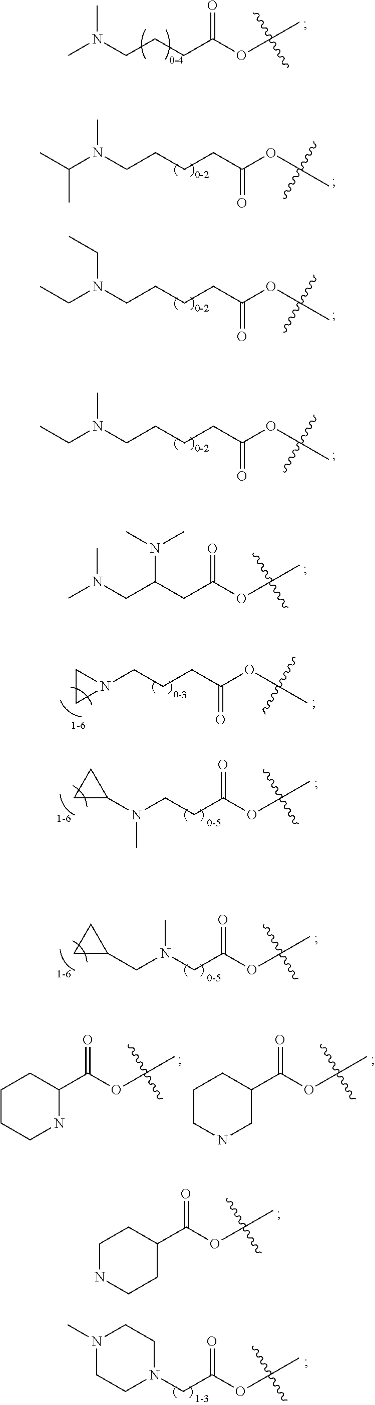

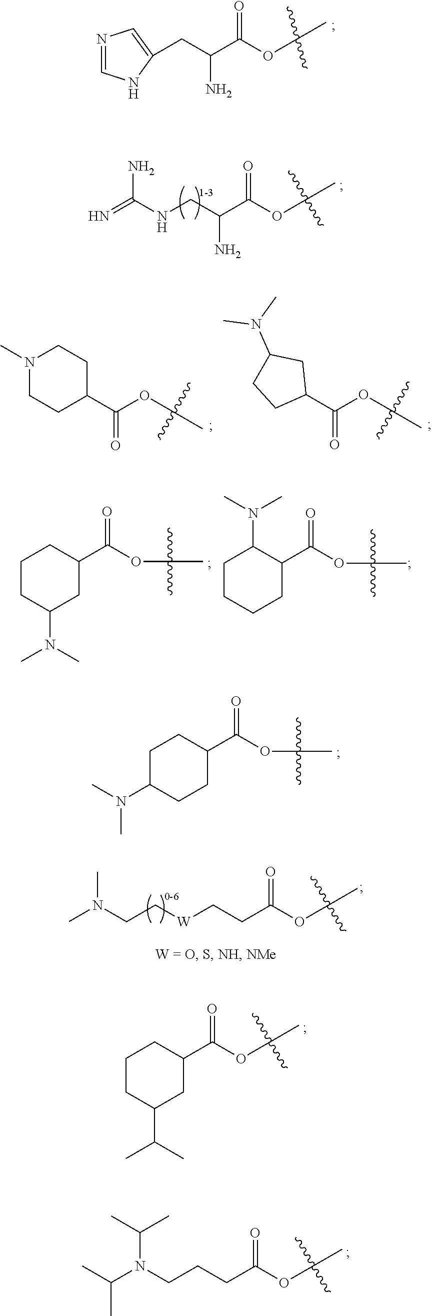

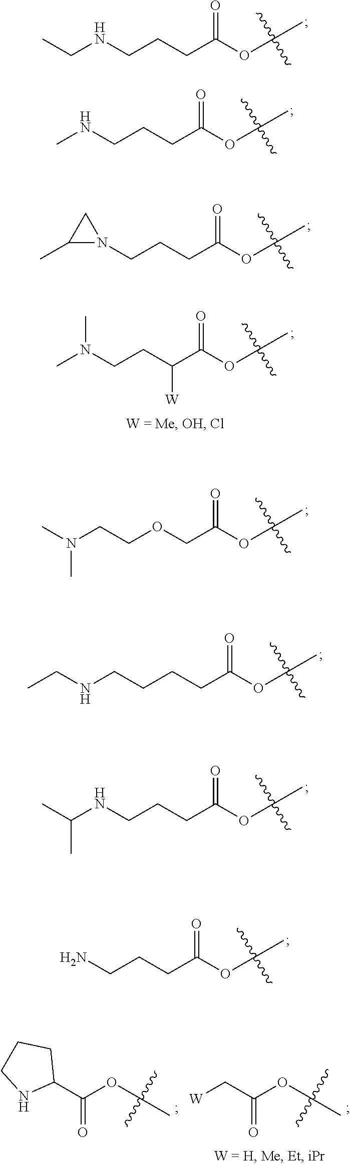

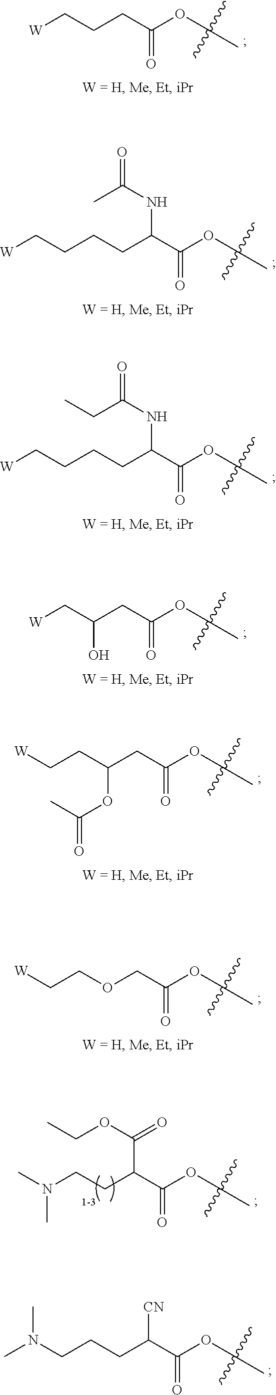

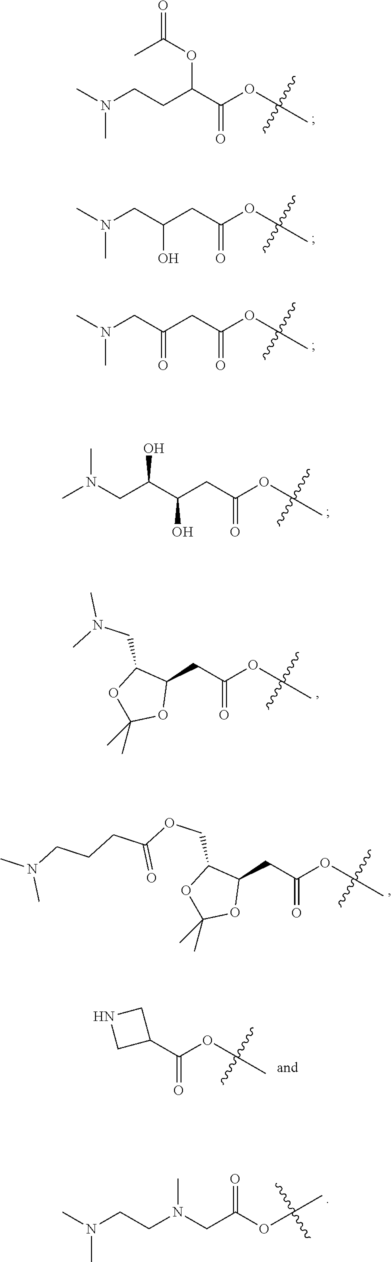

In some embodiments, the ionizable lipid is Lipid 1 (LP1),

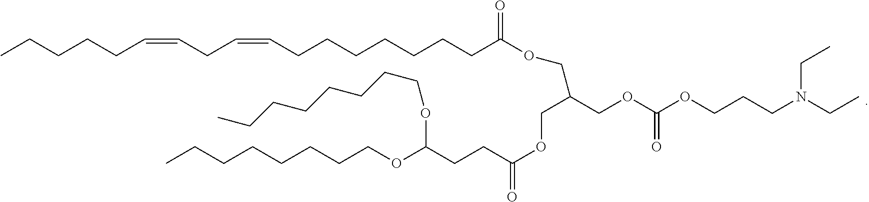 [SMILES: CCCCC/C=C\C/C=C\CCCCCCCC(=O)OCC(COC(=O)CCC(OCCCCCCCC)OCCCCCCCC)COC(=O)OCCCN(CC)CC]

In some embodiments, Lipid 1 may be synthesized according to WO2015/095340 (e.g., pp. 84-86).

In some embodiments, ionizable lipids suitable for use in the LNPs described herein are biodegradable in vivo. In some embodiments, the ionizable lipids have low toxicity (e.g., are tolerated in animal models without adverse effect in amounts of greater than or equal to 10 mg/kg based on weight of nucleic acid cargo, or in humans without serious adverse effects in amounts of greater than or equal to 1 mg/kg based on weight of nucleic acid cargo).

In certain embodiments, LNPs comprising an ionizable lipid described herein, following administration, has at least 75% of the ionizable lipid cleared from the plasma within 8, 10, 12, 24, or 48 hours or within 3, 4, 5, 6, 7, or 10 days. In certain embodiments, LNPs comprising an ionizable lipid described herein and a nucleic acid cargo, following administration, has at least 50% of the nucleic acid cargo cleared from the plasma within 8, 10, 12, 24, or 48 hours or within 3, 4, 5, 6, 7, or 10 days. In certain embodiments, LNPs comprising an ionizable lipid described herein, following administration, has at least 50% of the LNP cleared from the plasma within 8, 10, 12, 24, or 48 hours or within 3, 4, 5, 6, 7, or 10 days. In some embodiments, the clearance of the LNP is measured by the level of a lipid, nucleic acid (e.g., circular RNA), or protein component comprised in the LNP. Lipid clearance may be measured as described in literature. See Maier, M. A., et al. Biodegradable Lipids Enabling Rapidly Eliminated Lipid Nanoparticles for Systemic Delivery of RNAi Therapeutics. Mol. Ther. 2013, 21 (8), 1570-78 (“Maier”).

### Additional Lipids

“Neutral lipids” suitable for use in a lipid composition of the disclosure include, for example, a variety of neutral, uncharged or zwitterionic lipids. Examples of neutral phospholipids suitable for use in the present disclosure include, but are not limited to, 5-heptadecylbenzene-1,3-diol (resorcinol), dipalmitoylphosphatidylcholine (DPPC), distearoylphosphatidylcholine (DSPC), pohsphocholine (DOPC), dimyristoylphosphatidylcholine (DMPC), phosphatidylcholine (PLPC), 1,2-distearoyl-sn-glycero-3-phosphocholine (DAPC), phosphatidylethanolamine (PE), egg phosphatidylcholine (EPC), dilauryloylphosphatidylcholine (DLPC), dimyristoylphosphatidylcholine (DMPC), 1-myristoyl-2-palmitoyl phosphatidylcholine (MPPC), 1-palmitoyl-2-myristoyl phosphatidylcholine (PMPC), 1-palmitoyl-2-stearoyl phosphatidylcholine (PSPC), 1,2-diarachidoyl-sn-glycero-3-phosphocholine (DBPC), 1-stearoyl-2-palmitoyl phosphatidylcholine (SPPC), 1,2-dieicosenoyl-sn-glycero-3-phosphocholine (DEPC), palmitoyloleoyl phosphatidylcholine (POPC), lysophosphatidyl choline, dioleoyl phosphatidylethanolamine (DOPE), dilinoleoylphosphatidylcholine di stearoylphosphatidylethanolamine (DSPE), dimyristoyl phosphatidylethanolamine (DMPE), dipalmitoyl phosphatidylethanolamine (DPPE), palmitoyloleoyl phosphatidylethanolamine (POPE), lysophosphatidylethanolamine and combinations thereof. In one embodiment, the neutral phospholipid may be selected from the group consisting of distearoylphosphatidylcholine (DSPC) and dimyristoyl phosphatidyl ethanolamine (DMPE). In some embodiments, the neutral phospholipid may be distearoylphosphatidylcholine (DSPC). Neutral lipids function to stabilize and improve processing of the LNPs.

“Helper lipids” are lipids that are capable of enhancing transfection (e.g., transfection of the nanoparticle including the biologically active agent). The mechanism by which the helper lipid enhances transfection, in some embodiments, includes enhancing particle stability. Accordingly, in some embodiments, helper lipids described herein are capable of enhancing the stability of LNPs. In certain embodiments, the helper lipid enhances membrane fusogenicity. Helper lipids include steroids, sterols, and alkyl resorcinols. In some embodiments, helper lipids suitable for use in the present disclosure include, but are not limited to, cholesterol, 5-heptadecylresorcinol, and cholesterol hemisuccinate. In some embodiments, the helper lipid may be cholesterol. In some embodiments, the helper lipid may be cholesterol hemisuccinate.

“Stealth lipids” are lipids that are capable of altering the length of time the nanoparticles can exist in vivo (e.g., in the blood). Stealth lipids may, in some embodiments, assist in the formulation process by, for example, reducing particle aggregation and controlling particle size. Stealth lipids used herein may, in some embodiments, modulate pharmacokinetic properties of the LNP. Stealth lipids suitable for use in a lipid composition of the disclosure include, but are not limited to, stealth lipids having a hydrophilic head group linked to a lipid moiety. Stealth lipids suitable for use in a lipid composition of the present disclosure and information about the biochemistry of such lipids can be found in Romberg et al., Pharmaceutical Research, Vol. 25, No. 1, 2008, pg. 55-71 and Hoekstra et al., Biochimica et Biophysica Acta 1660 (2004) 41-52. Additional suitable PEG lipids are disclosed, e.g., in WO 2006/007712.

Embodiments of the present disclosure also provide lipid compositions described according to the respective molar ratios of the component lipids in the formulation. In some embodiments, the mol-% of the ionizable lipid may be from about 30 mol-% to about 60 mol-%. In some embodiments, the mol-% of the ionizable lipid may be from about 35 mol-% to about 55 mol-%. In some embodiments, the mol-% of the ionizable lipid may be from about 40 mol-% to about 50 mol-%. In some embodiments, the mol-% of the ionizable lipid may be from about 42 mol-% to about 47 mol-%. In some embodiments, the mol-% of the ionizable lipid may be about 45%. In some embodiments, the ionizable lipid mol-% of the LNP batch will be+30%, +25%, +20%, +15%, +10%, +5%, or +2.5% of the target mol-%. In certain embodiments, LNP inter-lot variability of the mol-% of the ionizable lipid is less than 15%, less than 10% or less than 5%.

In some embodiments, the mol-% of the helper lipid may be from about 30 mol-% to about 60 mol-%. In some embodiments, the mol-% of the helper lipid may be from about 35 mol-% to about 55 mol-%. In some embodiments, the mol-% of the helper lipid may be from about 40 mol-% to about 50 mol-%. In some embodiments, the mol-% of the helper lipid may be from about 41 mol-% to about 46 mol-%. In some embodiments, the mol-% of the helper lipid may be about 44 mol-%. In some embodiments, the helper mol-% of the LNP batch will be+30%, +25%, +20%, +15%, +10%, +5%, or +2.5% of the target mol-%. In certain embodiments, LNP inter-lot variability mol-% of the helper lipid is less than 15%, less than 10% or less than 5%.

In some embodiments, the mol-% of the neutral lipid may be from about 1 mol-% to about 20 mol-%. In some embodiments, the mol-% of the neutral lipid may be from about 5 mol-% to about 15 mol-%. In some embodiments, the mol-% of the neutral lipid may be from about 7 mol-% to about 12 mol-%. In some embodiments, the mol-% of the neutral lipid may be about 9 mol-%. In some embodiments, the neutral lipid mol-% of the LNP batch will be ±30%, ±25%, ±20%, ±15%, ±10%, ±5%, or ±2.5% of the target mol-%. In certain embodiments, LNP inter-lot variability mol-% of the neutral lipid is less than 15%, less than 10% or less than 5%.

In one embodiment, the mol-% of the stealth lipid may be from about 1 mol-% to about 10 mol-%. In some embodiments, the mol-% of the stealth lipid may be from about 1 mol-% to about 5 mol-%. In some embodiments, the mol-% of the stealth lipid may be from about 1 mol-% to about 3 mol-%. In some embodiments, the mol-% of the stealth lipid may be about 2 mol-%. In some embodiments, the mol-% of the stealth lipid may be about 1 mol-%. In some embodiments, the stealth lipid mol-% of the LNP batch will be ±30%, ±25%, ±20%, ±15%, ±10%, ±5%, or ±2.5% of the target mol-%. In certain embodiments, LNP inter-lot variability mol-% of the stealth lipid less than 15%, less than 10% or less than 5%.

Embodiments of the present disclosure also provide lipid compositions described according to the ratio between the positively charged amine groups of the ionizable lipid (N) and the negatively charged phosphate groups (P) of the nucleic acid to be encapsulated. This may be mathematically represented by the equation N/P. In some embodiments, the N/P ratio may be from about 0.5 to about 100. In some embodiments, the N/P ratio may be from about 1 to about 50. In some embodiments, the N/P ratio may be from about 1 to about 25. In some embodiments, the N/P ratio may be from about 1 to about 10. In some embodiments, the N/P ratio may be from about 1 to about 7. In some embodiments, the N/P ratio may be from about 3 to about 5. In some embodiments, the N/P ratio may be from about 4 to about 5. In some embodiments, the N/P ratio may be about 4. In some embodiments, the N/P ratio may be about 4.5. In some embodiments, the N/P ratio may be about 5.

The LNPs disclosed herein have a size of about 30 to about 200 nm. In some embodiments, the LNPs have a size of about 50 to about 150 nm. In some embodiments, the LNPs have a size of about 50 to about 100 nm. Unless indicated otherwise, all sizes referred to herein are the average sizes (diameters) of the fully formed nanoparticles, as measured by dynamic light scattering on a Malvern Zetasizer. For said measurement, the nanoparticle sample is diluted in phosphate buffered saline (PBS) so that the count rate is approximately 200-400 kcts, and the data is presented as a weighted-average of the intensity measure. In some embodiments, the LNPs are formed with an average encapsulation efficiency ranging from about 50% to about 100%. In some embodiments, the LNPs are formed with an average encapsulation efficiency ranging from about 50% to about 70%. In some embodiments, the LNPs are formed with an average encapsulation efficiency ranging from about 70% to about 90%. In some embodiments, the LNPs are formed with an average encapsulation efficiency ranging from about 90% to about 100%. In some embodiments, the LNPs are formed with an average encapsulation efficiency ranging from about 75% to about 95%.

In some embodiments, additional lipid agents may be formulated into the LNPs to assist in the RNA polynucleotide (e.g., circular or linear RNA) encapsulation. In general, the ionizable lipid of a LNP is responsible for ion pairing with the negatively charged RNA polynucleotide to drive encapsulation. This process can be aided by the addition of encapsulation agents to facilitate more efficient interactions with the RNAs and lipids, reducing the potential for partially encapsulated RNA. Encapsulated agents may comprise or consist of, for example, non-cationic lipids, ionizable lipids, small molecules with basic pKas, cationic peptides, cationic polymer, ionizable polymers, ethyl lauryl arginate (ELA), or another encapsulation agent available in the art. Exemplary agents are further described in PCT/US2019/052160 and is incorporated by reference herein.

Therapeutic uses

In some embodiments, compositions containing circular RNA may be useful for delivering therapeutic proteins to muscle for the treatment or prevention of muscle-based diseases or disorders. In some instances, the disease or disorder is a form of myopathy, dystrophy, or metabolic disease. Examples of muscular dystrophies include Duchenne, Becker, Facioscapulohumeral, Myotonic, Congenital, Distal, Emery-Dreifuss, Oculopharyngeal, and Limb Girdle. Examples of congenital dystrophies include Central Core, Myotubular, Nemaline, Ullrich/Bethlem, RyR1. Metabolic muscle diseases include Mitochondrial Myopathy, Pompe Disease, McArdles Disease, and Carnitine Palmitoyl Transferase Deficiency. Muscle diseases may be treated by administering a protein that restores or enhances a missing or defective function associated with the disease. For example, dystrophin or its variants can be used to restore function in muscular dystrophy. Collagen Type IV is another protein that may be used to treat muscle disease.

The therapeutic compositions described herein may be administered as a single dose, or in multiple doses, to achieve a desired level of protein expression in muscle tissue. In some embodiments, the therapy is administered in at least 1, 2, 3, 4, or 5 doses. Such doses many be administered approximately 1, 2, 3, or 4 weeks, or 1, 2, 3, 4, 5, or 6 months apart.

Muscle proteins

In some embodiments, the muscle protein or protein complex may be a non-human, human, chimeric, or humanized muscle protein or protein complex. In some embodiments, the muscle protein or protein complex is in whole or in part a smooth, skeletal, or cardiac muscle protein or protein complex.

### Dystrophins

As described herein, the therapeutic protein encoded by the nucleic acid cargo in the present invention may comprise or consist of a dystrophin protein, protein complex, or fragment thereof. In some embodiments, the dystrophin protein comprises a full-length dystrophin (427 kD protein). In some embodiments, the full-length dystrophin comprises an actin-binding amino-terminal domain (ABD1), a central rod domain, a cysteine-rich domain and a carboxyl-terminus. In some embodiments, the central rod domain comprises 24 spectrin-like repeats and four hinges. In some embodiments, the dystrophin protein may be a naturally occurring full-length dystrophin or a synthetic dystrophin.

In other embodiments, the dystrophin protein may comprise a dystrophin variant. “Variants” are proteins comprising one or more amino acid mutations, deletions, or insertions compared to a full-length functional protein or complex available in the art. In some embodiments, dystrophin variants may be truncations of a full-length functional dystrophin protein or complex by removing one or more amino acids at either end of the amino acid (e.g., at the C terminus or the N terminus). In another embodiment, the dystrophin variant may be altered by removal of one or more spectrin-like repeats and/or hinges. In some embodiments, the dystrophin variant comprises 6, 7, 8, 9, 10, 11, 12, 13, 14, 15, 16, 17, 18, 19, 20, 21, 22, 23, or 24 spectrin-like repeats.

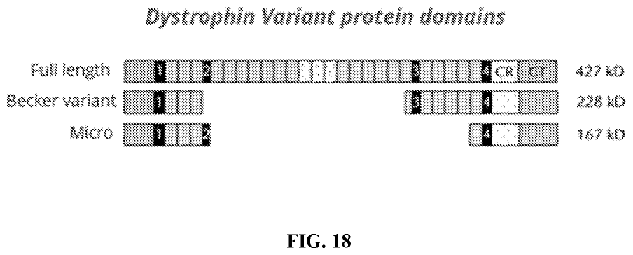

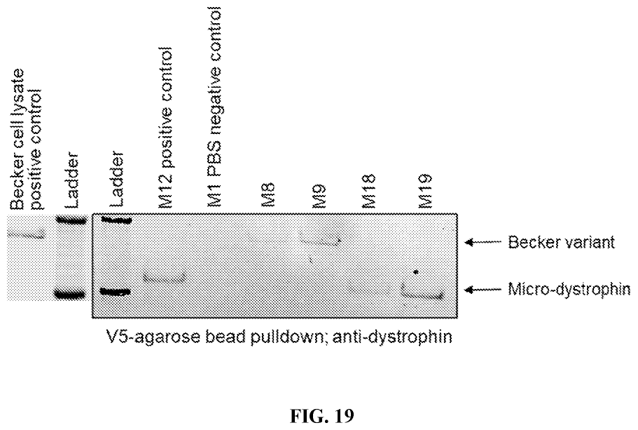

In some embodiments, the dystrophin variant may comprise hinge 1, hinge 2, hinge 3 and/or hinge 4 domain. In some embodiments, the dystrophin variant may lack the cysteine variant present in the naturally occurring full-length dystrophin. In some embodiments, the dystrophin variant may further comprise a synthrophin-binding domain. In certain embodiments, the dystrophin variant comprises at least about 2000 amino acids in length. In some embodiments, the dystrophin variant comprises about 2000, 2100, 2200, 2300, 2400, 2500, 2600, 2700, 2800, 2900, 3000, 3100, 3200, 3300, 3400, or 3500 amino acids in length. In some embodiments, the dystrophin variant comprises or essentially consists of a sequence selected from SEQ ID NOs: 18, 20, 24, 26, 28, and 30. In some embodiments, the dystrophin variant comprises a sequence at least 80%, 85%, 90%, 91%, 92%, 93%, 94%, 95%, 96%, 97%, 98%, or 99% identical to a sequence selected from SEQ ID NOs: 18, 20, 24, 26, 28, and 30. In some embodiments, the dystrophin variant comprises a Becker variant. For example, a Becker variant may comprise or consist essentially of the Becker variant in FIG. 18. In some embodiments, the Becker variant comprises or essentially consists of SEQ ID NO: 22 or SEQ ID NO: 32. In some embodiments, the Becker variant comprises a sequence at least 80%, 85%, 90%, 91%, 92%, 93%, 94%, 95%, 96%, 97%, 98%, or 99% identical to SEQ ID NO: 22 or SEQ ID NO: 32.

Exemplary dystrophin variants are described in Dongsheng Duan, (2018) Systemic AAV Micro-dystrophin Gene Therapy for Duchenne Muscular Dystrophy. Molecular Therapy. 26 (10): 2337-2356 (illustrating micro-dystrophin and mini-dystrophin); Li et al., Protein Trans-Splicing as Means for Viral Vector-Mediated In Vivo Gene Therapy. Hum Gene Ther. 2008 September; 19 (9): 958-964 (illustrating split-inteins catalytic methods for producing varying dystrophin constructs); and Quan Gao & Elizabeth McNally, The Dystrophin Complex: structure, function and implications for therapy, Compr Physiol. 2015 Hul 1; 5 (3): 1223-1239. (providing other modifications to the dystrophin protein) all of which are incorporated by reference in their entirety herein.

In some embodiments, a dystrophin protein, protein complex, or fragment thereof is selected from Table 1.

In certain embodiments, the dystrophin protein may comprise one or more dystrophin-associated proteins. In some embodiments, the dystrophin-associated protein may comprise an extracellular, transmembrane and/or cytoplasmic dystrophin-associated protein. In some embodiments, the extracellular dystrophin-associated protein comprises α-dystroglycan. In some embodiments, the transmembrane dystrophin-associated protein comprises β-dystroglycan, sarcoglycans, or sarcospan. In some embodiments, the cytoplasmic dystrophin-associated protein comprises dystrophin, dystrobrevin, syntrophins, or neuronal nitric oxide synthase.

Other muscle proteins

In certain embodiments, the present disclosure provides for tissue specific muscle protein or protein complexes available in the art. In some embodiments, the muscle protein or protein complex produced by the circular RNA polynucleotide comprises a smooth muscle myosin, actin, tropomyosin, calponin, caldesmon, or fragment or isoform thereof. In other embodiments, the muscle protein or protein complex produced comprises a skeletal or muscular sarcolemmal protein, laminin, collagen, action, myosin, myofibrillar protein, or intermediate protein, or a fragment, variant or isoform thereof.

### EXAMPLES

Wesselhoeft et al. (2019) RNA Circularization Diminishes Immunogenicity and Can Extend Translation Duration In Vivo. Molecular Cell. 74 (3), 508-520 and Wesselhoeft et al. (2018) Engineering circular RNA for Potent and Stable Translation in Eukaryotic Cells. Nature Communications. 9, 2629, are incorporated by reference in their entirety.

The invention is further described in detail by reference to the following examples but are not intended to be limited to the following examples. These examples encompass any and all variations of the illustrations with the intention of providing those of ordinary skill in the art with complete disclosure and description of how to make and use the subject invention and are not intended to limit the scope of what is regarded as the invention.

### Example 1

### Example 1A: Synthesis of lipids

Synthesis of representative ionizable lipids of the invention is described in PCT applications PCT/US2014/070882, PCT/US2016/052352, PCT/US2016/068300, PCT/US2010/061058, PCT/US2018/058555, PCT/US2018/053569, PCT/US2017/028981, PCT/US2019/025246, PCT/US2018/035419, PCT/US2019/015913, PCT/US2020/063494, PCT/2020/034418 and US applications with publication numbers 20170210697, 20190314524, 20190321489, and 20190314284, the contents of each of which are incorporated herein by reference in their entireties.

### Example 1B: Production of lipid nanoparticle compositions

In order to investigate safe and efficacious lipid nanoparticle (LNP) compositions for use in the delivery of circular RNA to cells, a range of formulations are prepared and tested. Specifically, the particular elements and ratios thereof in the lipid component of nanoparticle compositions are optimized.

LNPs containing circular RNAs can be made in a 1 fluid stream or with mixing processes such as microfluidics and T-junction mixing of two fluid streams, one of which contains the circular RNAs and the other has the lipid components.

Lipid compositions are prepared by combining an ionizable lipid, optionally a helper lipid (such as DOPE, DSPC, or oleic acid obtainable from Avanti Polar Lipids, Alabaster, AL), a PEG lipid (such as 1,2-dimyristoyl-sn-glycerol methoxypolyethylene glycol, also known as PEG-DMG, obtainable from Avanti Polar Lipids, Alabaster, AL), and a structural lipid such as cholesterol at concentrations of about, e.g., 40 or 50 mM in a solvent, e.g., ethanol. Solutions should be refrigerated for storage at, for example,−20° C. Lipids are combined to yield desired molar ratios (see, for example, in the table below) and diluted with water and ethanol to a final lipid concentration of e.g., between about 5.5 mM and about 25 mM.

| Formulation | |
|---|---|
| Number | Description |
| 1 | Aliquots of 50 mg/mL ethanolic solutions of C12-200, DOPE, Chol and DMG- |
| | PEG2K (40:30:25:5) are mixed and diluted with ethanol to 3 mL final volume. |
| | Separately, an aqueous buffered solution (10 mM citrate/150 mM NaCl, pH 4.5) |
| | of circRNA is prepared from a 1 mg/mL stock. The lipid solution is injected |
| | rapidly into the aqueous circRNA solution and shaken to yield a final suspension |
| | in 20% ethanol. The resulting nanoparticle suspension is filtered, diafiltrated |
| | with 1 × PBS (pH 7.4), concentrated and stored at 2-8° C. |
| 2 | Aliquots of 50 mg/mL ethanolic solutions of DODAP, DOPE, cholesterol and |
| | DMG-PEG2K (18:56:20:6) are mixed and diluted with ethanol to 3 mL final |
| | volume. Separately, an aqueous buffered solution (10 mM citrate/150 mM |
| | NaCl, pH 4.5) of EPO circRNA is prepared from a 1 mg/mL stock. The lipid |
| | solution is injected rapidly into the aqueous circRNA solution and shaken to |
| | yield a final suspension in 20% ethanol. The resulting nanoparticle suspension |
| | is filtered, diafiltrated with 1 × PBS (pH 7.4), concentrated and stored at 2-8° C. |
| | Final concentration = 1.35 mg/mL EPO circRNA (encapsulated). Zave = 75.9 nm |
| | (Dv(50) = 57.3 nm; Dv(90) = 92.1 nm). |
| 3 | Aliquots of 50 mg/mL ethanolic solutions of HGT4003, DOPE, cholesterol and |
| | DMG-PEG2K (50:25:20:5) are mixed and diluted with ethanol to 3 mL final |
| | volume. Separately, an aqueous buffered solution (10 mM citrate/150 mM |
| | NaCl, pH 4.5) of circRNA is prepared from a 1 mg/mL stock. The lipid solution |
| | is injected rapidly into the aqueous circRNA solution and shaken to yield a final |
| | suspension in 20% ethanol. The resulting nanoparticle suspension is filtered, |
| | diafiltrated with 1 × PBS (pH 7.4), concentrated and stored at 2-8° C. |
| 4 | Aliquots of 50 mg/mL ethanolic solutions of ICE, DOPE and DMG-PEG2K |
| | (70:25:5) are mixed and diluted with ethanol to 3 mL final volume. Separately, |
| | an aqueous buffered solution (10 mM citrate/150 mM NaCl, pH 4.5) of circRNA |
| | is prepared from a 1 mg/mL stock. The lipid solution is injected rapidly into the |
| | aqueous circRNA solution and shaken to yield a final suspension in 20% |
| | ethanol. The resulting nanoparticle suspension is filtered, diafiltrated with |
| | 1 × PBS (pH 7.4), concentrated and stored at 2-8° C. |
| 5 | Aliquots of 50 mg/mL ethanolic solutions of HGT5000, DOPE, cholesterol and |
| | DMG-PEG2K (40:20:35:5) are mixed and diluted with ethanol to 3 mL final |
| | volume. Separately, an aqueous buffered solution (10 mM citrate/150 mM |
| | NaCl, pH 4.5) of EPO circRNA is prepared from a 1 mg/mL stock. The lipid |
| | solution is injected rapidly into the aqueous circRNA solution and shaken to |
| | yield a final suspension in 20% ethanol. The resulting nanoparticle suspension |
| | is filtered, diafiltrated with 1 × PBS (pH 7.4), concentrated and stored at 2-8° C. |
| | Final concentration = 1.82 mg/mL EPO mRNA (encapsulated). Zave = 105.6 nm |
| | (Dv(50) = 53.7 nm; Dv(90) = 157 nm). |
| 6 | Aliquots of 50 mg/mL ethanolic solutions of HGT5001, DOPE, cholesterol and |
| | DMG-PEG2K (40:20:35:5) are mixed and diluted with ethanol to 3 mL final |
| | volume. Separately, an aqueous buffered solution (10 mM citrate/150 mM |
| | NaCl, pH 4.5) of EPO circRNA is prepared from a 1 mg/mL stock. The lipid |
| | solution is injected rapidly into the aqueous circRNA solution and shaken to |
| | yield a final suspension in 20% ethanol. The resulting nanoparticle suspension |
| | is filtered, diafiltrated with 1 × PBS (pH 7.4), concentrated and stored at 2-8° C. |
| 7 | Aliquots of 50 mg/mL ethanolic solutions of HGT5001, DOPE, cholesterol and |
| | DMG-PEG2K (35:16:46.5:2.5) are mixed and diluted with ethanol to 3 mL final |
| | volume. Separately, an aqueous buffered solution (10 mM citrate/150 mM |
| | NaCl, pH 4.5) of EPO circRNA is prepared from a 1 mg/mL stock. The lipid |
| | solution is injected rapidly into the aqueous circRNA solution and shaken to |
| | yield a final suspension in 20% ethanol. The resulting nanoparticle suspension |
| | is filtered, diafiltrated with 1 × PBS (pH 7.4), concentrated and stored at 2-8° C. |
| 8 | Aliquots of 50 mg/mL ethanolic solutions of HGT5001, DOPE, cholesterol and |
| | DMG-PEG2K (40:10:40:10) are mixed and diluted with ethanol to 3 mL final |
| | volume. Separately, an aqueous buffered solution (10 mM citrate/150 mM |
| | NaCl, pH 4.5) of EPO circRNA is prepared from a 1 mg/mL stock. The lipid |
| | solution is injected rapidly into the aqueous circRNA solution and shaken to |
| | yield a final suspension in 20% ethanol. The resulting nanoparticle suspension |
| | is filtered, diafiltrated with 1 × PBS (pH 7.4), concentrated and stored at 2-8° C. |
| 9 | Aliquots of 50 mg/mL ethanolic solutions of LP1, DSPC, cholesterol and DMG- |
| | PEG2K (45:9:44:2) are mixed and diluted with ethanol to 3 mL final volume. |
| | Separately, an aqueous buffered solution (50 mM Na Acetate, pH 4.5) of FLuc, |
| | hEPO, micro-dystrophin, mini-dystrophin, or dystrophin circRNA is prepared |
| | from a 1 mg/mL stock. The lipid solution is injected rapidly into the aqueous |
| | circRNA solution and shaken to yield a final suspension in 25% ethanol. The |
| | resulting nanoparticle suspension is filtered, diafiltrated with 1 × PBS (pH 7.4), |
| | concentrated and stored at 2-8° C. |
| 10 | Aliquots of 50 mg/mL ethanolic solutions of LP1, DSPC, cholesterol and |
| | DMG-PEG2K, and DMSO solution of Ethyl Lauroyl Arginate |
| | (38.25:7.65:37.4:1.7:15) are mixed and diluted with ethanol to 3 mL final |
| | volume. Separately, an aqueous buffered solution (50 mM Bis-Tris, pH 7.0) of |
| | FLuc, hEPO, micro-dystrophin, mini-dystrophin, or dystrophin circRNA is |
| | prepared from a 1 mg/mL stock. The lipid solution is injected rapidly into the |
| | aqueous circRNA solution and shaken to yield a final suspension in 25% |
| | ethanol. The resulting nanoparticle suspension is filtered, diafiltrated with |
| | 1 × PBS (pH 7.4), concentrated and stored at 2-8° C. |
| 11 | Aliquots of 50 mg/mL ethanolic solutions of SM-102, DSPC, cholesterol and |
| | DMG-PEG2K (50:10:38.5:1.5) are mixed and diluted with ethanol to 3 mL |
| | final volume. Separately, an aqueous buffered solution (6.25 mM Na Acetate, |
| | pH 4.5) of FLuc, hEPO, micro-dystrophin, mini-dystrophin, or dystrophin |
| | circRNA is prepared from a 1 mg/mL stock. The lipid solution is injected |
| | rapidly into the aqueous circRNA solution and shaken to yield a final |
| | suspension in 25% ethanol. The resulting nanoparticle suspension is filtered, |
| | diafiltrated with 1 × PBS (pH 7.4), concentrated and stored at 2-8° C. |
| 12 | Aliquots of 50 mg/mL ethanolic solutions of SM-102, DSPC, cholesterol and |
| | DMG-PEG2K, and DMSO solution of Ethyl Lauroyl Arginate |
| | (42.5:8.5:32.73:1.275:15) are mixed and diluted with ethanol to 3 mL final |
| | volume. Separately, an aqueous buffered solution (20 mM Na Acetate, pH 4.5) |
| | of FLuc, hEPO, micro-dystrophin, mini-dystrophin, or dystrophin circRNA is |
| | prepared from a 1 mg/mL stock. The lipid solution is injected rapidly into the |
| | aqueous circRNA solution and shaken to yield a final suspension in 25% |
| | ethanol. The resulting nanoparticle suspension is filtered, diafiltrated with |
| | 1 × PBS (pH 7.4), concentrated and stored at 2-8° C. |

Solutions of the circular RNA at concentrations ranging from 0.175 mg/ml to 0.440 mg/mL in deionized water are diluted in a buffer, e.g., 50 mM sodium citrate buffer at a pH between 3 and 4 to form a stock solution. Alternatively, solutions of the circRNA at concentrations ranging from 0.175 mg/ml to 0.440 mg/mL in deionized water are diluted in a buffer, e.g., 6.25 mM sodium acetate buffer at a pH between 3 and 4.5 to form a stock solution. Alternatively, solutions of the circRNA at concentrations ranging from 0.175 mg/ml to 0.440 mg/mL in deionized water are diluted in a buffer, e.g., 50 mM Bis-Tris buffer at a pH 7 to form a stock solution. The circular RNA solution is then combined with the lipid solution at lipid component to

10 circRNA wt: wt ratios between about 5:1 and about 50:1, by rapidly injecting the lipid solution using, e.g., a NanoAssemblr microfluidic based system at flow rates between about 10 mL/min and about 18 ml/min or between about 5 mL/min and about 18 mL/min into the circRNA solution, and producing a suspension with a water to ethanol ratio between about 1:1 and about 4:1.

LNP compositions can be processed by dialysis to remove ethanol and achieve buffer exchange. Formulations are dialyzed twice against phosphate buffered saline (PBS), pH 7.4, at volumes 200 times that of the primary product using Slide-A-Lyzer cassettes (Thermo Fisher Scientific Inc., Rockford, IL) with a molecular weight cutoff of 10 kDa or 20 kDa. The formulations are then dialyzed overnight at 4° C. The resulting nanoparticle suspension is filtered through 0.2 μm sterile filters (Sarstedt, Nümbrecht, Germany) into glass vials and sealed with crimp closures. Nanoparticle composition solutions of 0.01 mg/mL to 0.15 mg/mL (e.g., 0.5 mg/mL to 1.2 mg/mL) are generally obtained.

The method described above induces nano-precipitation and particle formation.

Alternative processes including, but not limited to, T-junction and direct injection, may be used to achieve the same nano-precipitation.

### Example 1C: Characterization of lipid nanoparticle compositions

A Zetasizer Nano ZS (Malvern Instruments Ltd, Malvern, Worcestershire, UK) can be used to determine the particle size, the polydispersity index (PDI) and the zeta potential of the nanoparticle compositions in 1×PBS in determining particle size and 15 mM PBS in determining zeta potential.

Ultraviolet-visible spectroscopy can be used to determine the concentration of circRNA in nanoparticle compositions. 100 μL of the diluted formulation in 1×PBS is added to 900 μL of a 4:1 (v/v) mixture of methanol and chloroform. After mixing, the absorbance spectrum of the solution is recorded, for example, between 230 nm and 330 nm on a DU 800 spectrophotometer (Beckman Coulter, Beckman Coulter, Inc., Brea, CA). The concentration of circRNA in the nanoparticle composition can be calculated based on the extinction coefficient of the circRNA used in the composition and on the difference between the absorbance at a wavelength of, for example, 260 nm and the baseline value at a wavelength of, for example, 330 nm.

A QUANT-IT™ RIBOGREEN® RNA assay (Invitrogen Corporation Carlsbad, CA) can be used to evaluate the encapsulation of circRNA by the nanoparticle composition. The samples are diluted to a concentration of approximately 5 μg/mL or 1 μg/mL in a TE buffer solution (10 mM Tris-HCl, 1 mM EDTA, pH 7.5). 50 μL of the diluted samples are transferred to a polystyrene 96 well plate and either 50 μL of TE buffer or 50 μL of a 2-4% Triton X-100 solution is added to the wells. The plate is incubated at a temperature of 37° C. for 15 minutes. The RIBOGREEN® reagent is diluted 1:100 or 1:200 in TE buffer, and 100 μL of this solution is added to each well. The fluorescence intensity can be measured using a fluorescence plate reader (Wallac Victor 1420 Multilabel Counter; Perkin Elmer, Waltham, MA) at an excitation wavelength of, for example, about 480 nm and an emission wavelength of, for example, about 520 nm. The fluorescence values of the reagent blank are subtracted from that of each of the samples and the percentage of free circRNA is determined by dividing the fluorescence intensity of the intact sample (without addition of Triton X-100) by the fluorescence value of the disrupted sample (caused by the addition of Triton X-100).

### Example 1D: In vivo formulation studies

In order to monitor how effectively various nanoparticle compositions deliver circRNA to targeted cells, different nanoparticle compositions including circRNA are prepared and administered to rodent populations. Mice are intravenously, intramuscularly, intraarterially, or intratumorally administered a single dose including a nanoparticle composition with a lipid nanoparticle formulation. In some instances, mice may be made to inhale doses. Dose sizes may range from 0.001 mg/kg to 10 mg/kg, where 10 mg/kg describes a dose including 10 mg of a circRNA in a nanoparticle composition for each 1 kg of body mass of the mouse. A control composition including PBS may also be employed.

Upon administration of nanoparticle compositions to mice, dose delivery profiles, dose responses, and toxicity of particular formulations and doses thereof can be measured by enzyme-linked immunosorbent assays (ELISA), bioluminescent imaging, or other methods. Time courses of protein expression can also be evaluated. Samples collected from the rodents for evaluation may include blood and tissue (for example, muscle tissue from the site of an intramuscular injection and internal tissue); sample collection may involve sacrifice of the animals.

Higher levels of protein expression induced by administration of a composition including a circRNA is indicative of higher circRNA translation and/or nanoparticle composition circRNA delivery efficiencies. As the non-RNA components are not thought to affect translational machineries themselves, a higher level of protein expression is likely indicative of a higher efficiency of delivery of the circRNA by a given nanoparticle composition relative to other nanoparticle compositions or the absence thereof.

### Example 2

### Example 2A: Synthesis of engineered circular RNA

oRNA precursors containing a permuted group I intron were transcribed from a plasmid template by in vitro transcription using T7 RNA polymerase. Precursors autocatalytically spliced to create oRNA and post-splicing intron fragments/segments. In vitro transcription reactions were purified by methods known to the art such as size-exclusion chromatography, reverse phase chromatography, ion exchange chromatography, affinity purification, affinity chromatography, enzymatic digestion by exonucleases such as RNase R and/or Xrnl, phosphatase treatment by enzymes such as calf intestinal phosphatase (CIP), alcohol precipitation, silica membrane purification, agarose or polyacrylamide gel extraction, ultrafiltration, and tangential flow filtration.

### Example 2B: Purification of circular RNA constructs using various purification methods

Human embryonic kidney 293 (HEK293) and human lung carcinoma A549 cells were transfected with:
a. products of an unpurified GLuc circular RNA splicing reaction,
b. products of RNase R digestion of the splicing reaction,
c. products of RNase R digestion and HPLC purification of the splicing reaction, or
d. products of RNase digestion, HPLC purification, and phosphatase treatment of the splicing reaction.

RNase R digestion of splicing reactions was insufficient to prevent cytokine release in A549 cells in comparison to untransfected controls. The addition of HPLC purification was also insufficient to prevent cytokine release, although there was a significant reduction in interleukin-6 (IL-6) and a significant increase in interferon-al (IFNα1) compared to the unpurified splicing reaction.

The addition of a phosphatase treatment after HPLC purification and before RNase R digestion dramatically reduced the expression of all upregulated cytokines assessed in A549 cells. Secreted monocyte chemoattractant protein 1 (MCP1), IL-6, IFNα1, tumor necrosis factor a (TNFα), and IFNγ inducible protein-10 (IP-10) fell to undetectable or un-transfected baseline levels.

There was no substantial cytokine release in HEK293 cells. A549 cells had increased GLuc expression stability and cell viability when transfected with higher purity circular RNA. Completely purified circular RNA had a stability phenotype similar to that of transfected 293 cells.

### Example 3

Protein expression of circular RNA in mouse myoblast cells.

Engineered circular RNAs encoding firefly luciferase protein were transfected into mouse C2C12 myoblasts using Lipofectamine 3000 at different cell densities (3K, 10K, 15K per well, 96 wells). The myoblast cells were dose with either 0, 50, 100, or 200 ng of the RNA construct. Protein expression data was collected at 24 hours post transfection.

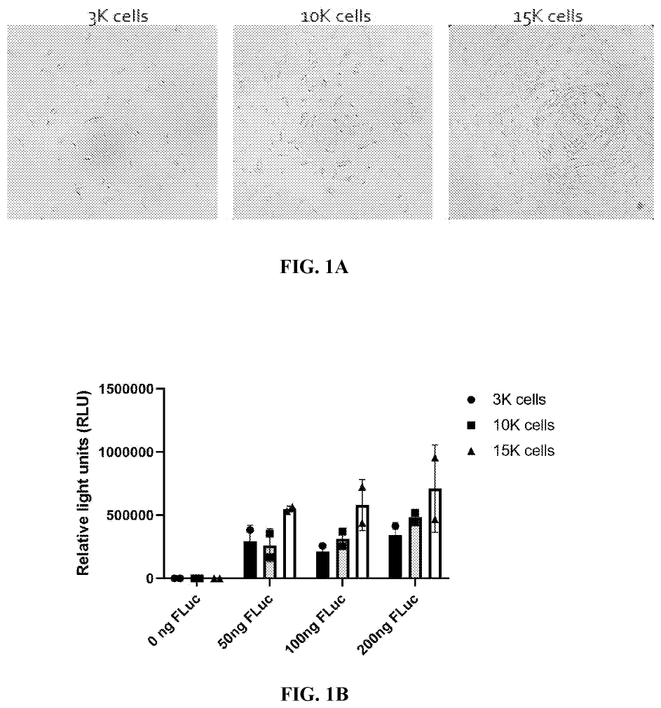

As seen in FIGS. 1A and 1B, transfected circular RNA constructs were able to produce protein expression in various different cell densities and for various different dosages in mouse myoblast.

### Example 4

Protein expression of circular RNA in human skeletal muscle myoblasts.

Engineered circular RNAs encoding firefly luciferase protein were transfected into human skeletal muscle myoblasts from three different donors using Lipofectamine 3000. The cells were plated at cell densities of either 10K or 30K cells per well. The cells were dosed at a concentration of 0 (control), 10, 25, 50, or 100 ng of the RNA construct. Expression was recorded 24 hours post transfection.

Engineered circular RNAs encoding firefly luciferase protein were transfected into human skeletal muscle myoblasts from three different donors. The cells were plated at cell densities of either 10K or 30K cells per well. The cells were dosed at a concentration of 0 (control), 10, 25, 50, or 100 ng the RNA construct. Expression was recorded 24 hours post transfection.

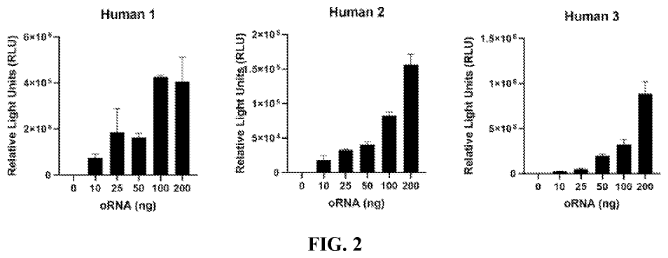

As seen in FIG. 2, circular RNA constructs were able to produce protein at different dosages in human skeletal muscle myoblast.

### Example 5

Expression in myotubes following transfection of circular RNA encoding firefly luciferase.

Engineered circular RNAs encoding firefly luciferase were transfected into differentiated human skeletal muscle myotubes using Lipofectamine 3000. The human skeletal muscle myotubes were collected from 2 different donors. The cells were plated in a 96 well plate with 10K cells per well density. Circular RNA constructs were then transfected into human skeletal muscle myotubes at either a 0, 10, 25, 50, 100, or 200 ng dosage. The cells were observed two days before the transfection date, on the date of transfection and 24 hours following transfection. Relative light units were collected 24 hours post transfection of the cell with the circular RNA constructs.

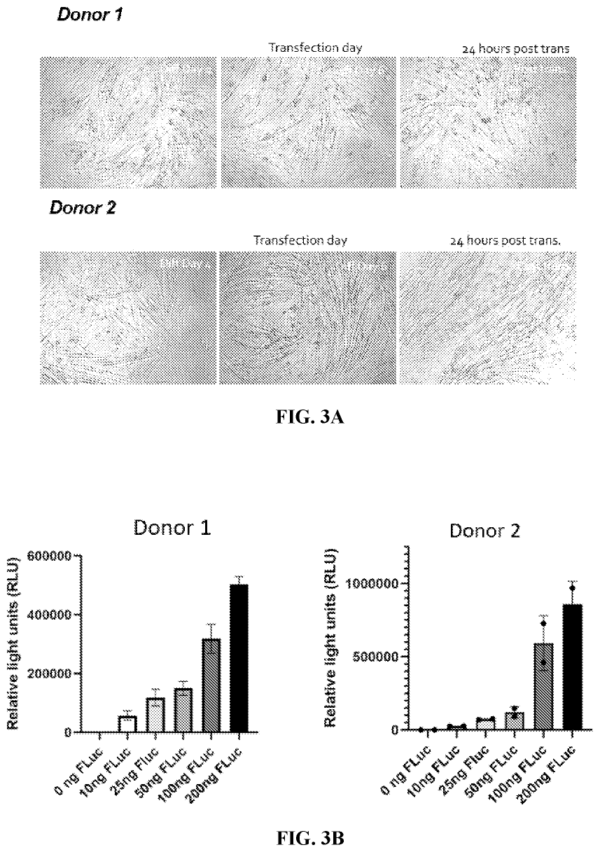

As seen in FIGS. 3A and 3B, circular RNA constructs were able to produce protein at different dosages in human skeletal muscle myotubes.

### Example 6

Expression of micro-dystrophin in human skeletal myotubes following transfection of lipid nanoparticles-circular constructs.

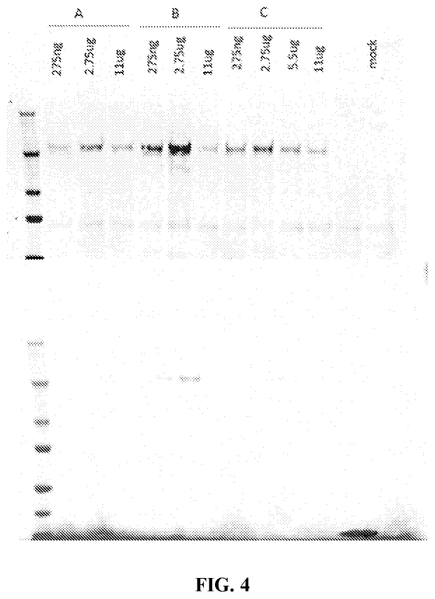

Circular RNA constructs encoding micro-dystrophin were encapsulated into lipid nanoparticles comprising Lipid 1 (LP1) and transfected into human skeletal myotubes with three different formulations (see below table) during a period of 5 days. oRNA was dosed at either 275 ng, 2.75 μg, or 11 μg. For comparison 550 ng of circular RNA-LNP was transfected, wherein the circular RNA encoded Gaussia luciferase. Micro-dystrophin expression for all three of the constructs was present in the human skeletal muscle myotubes post Western blot analysis as seen in FIG. 4.

| | | | | | | Ionizable lipid:Helper |
|---|---|---|---|---|---|---|
| | | | | | | Lipid:Cholesterol:PEG- |
| | | Ionizable | N:P | Helper | | lipid:Ethyl Lauryl |
| Group | Formulation | Lipid | Ratio | Lipid | PEG-lipid | Arginate (ELA) (mol %) |
| A | LP1 (4.5B) | LP1 | 4.5:1 | DSPC | DMG-PEG | 45:9:44:2:0 |
| | | | | | (2000) | |
| B | LP1 (4.5OB | LP1 | 4.5:1 | DSPC | DMG-PEG | 38.25:7.65:37.4:1.7:15 |
| | Neutral) | | | | (2000) | |
| C | LP1 (4.5OB | LP1 | 4.5:1 | DSPC | DMG-PEG | 38.25:7.65:37.4:1.7:15 |
| | Acidic) | | | | (2000) | |

### Example 7

HeLa Cell-Free translation of circular RNA encoding either full-length or micro-dystrophin.

RNA encoding either full length dystrophin or micro-dystrophin were added to a HeLa Cell-Free translation (CFT) assay. The cell-free assay was allowed to run for 90 minutes. Following the HeLa Cell-Free translation assay, the sample was analyzed using a Western blot, wherein the Western Blot comprised a 3-8% tris-acetate gel and a 20 μL sample load (15 μL CFT reaction product+5 μL 4x sample buffer). An anti-dystrophin antibody was used (Leica NCL-Dys2) for the Western blot analysis.

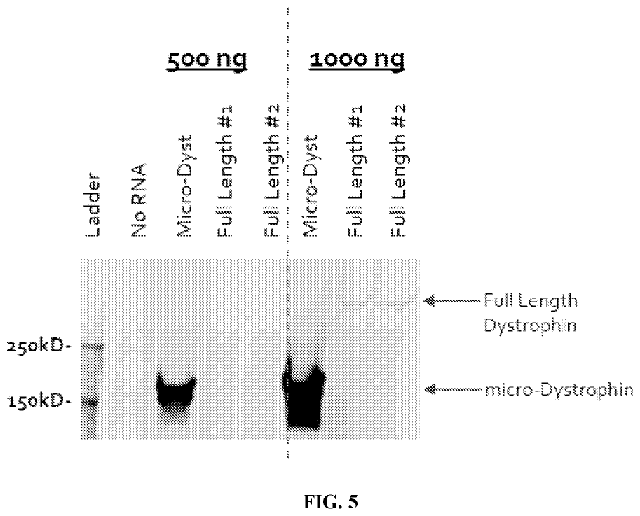

As seen in FIG. 5, micro-dystrophin was present at 150 kD for both input dosages; the full-length dystrophins were present at greater than 250 kD in the 1000 ng input dosage. Based on the Western blot, the micro-dystrophin constructs had a predicted molecular weight of 167 kD, while the full-length dystrophin had a predicted molecular weight of 427 kD.

### Example 8

In vitro protein expression in human skeletal muscle myotubes from transfection of circular RNA encoding mini-dystrophin encapsulated within various lipid formulations.

Circular RNAs were engineered to encode mini-dystrophin or Green Fluorescent Protein (GFP; positive transfection control) and encapsulated within lipid nanoparticles comprising either Lipid 1 (LP1) or a comparable lipid (see table below). Human skeletal muscle myotubes were plated at 15 k per well and allowed to differentiate for 5 days. The human skeletal muscle myotubes were dosed at either 275, 2750, or 5500 ng of the circular RNA-LNP construct. Protein was isolated 48 hours post transfection and analyzed using a Western blot.

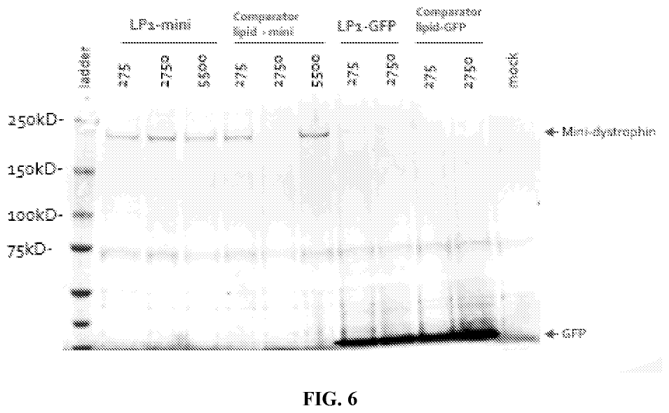

As seen in FIG. 6, mini-dystrophin expression was present for both types of lipid formulations.

| | | | | | | | Ionizable lipid:Helper |
|---|---|---|---|---|---|---|---|
| | Encoded | | Ionizable | N:P | Helper | | Lipid:Cholesterol:PEG- |
| Group | Protein | Formulation | Lipid | Ratio | Lipid | PEG-lipid | lipid (mol %):ELA |
| LP1-mini | Mini | LP1 | LP1 | 4.5:1 | DSPC | DMG-PEG | 38.25:7.65:37.4:1.7:15 |
| | dystrophin | (4.5OB) | | | | (2000) | |
| Comparator | Mini | CP1 (5.7OA) | Comparator | 5.7:1 | DSPC | DMG-PEG | 42.5:8.5:32.725:1.275:15 |
| lipid-mini | dystrophin | | lipid | | | (2000) | |
| LP1-GFP | GFP | LP1 | LP1 | 4.5:1 | DSPC | DMG-PEG | 38.25:7.65:37.4:1.7:15 |
| | | (4.5OB) | | | | (2000) | |
| Comparator | GFP | CP | Comparator | 5.7:1 | DSPC | DMG-PEG | 42.5:8.5:32.725:1.275:15 |
| lipid-GFP | | (5.7OA) | lipid | | | (2000) | |

### Example 9

In vivo protein expression in mdx mice following transfection of circular RNA encoding micro-dystrophin and encapsulated in different LNP formulations.

Engineered circular RNAs encoding micro-dystrophin were encapsulated into lipid nanoparticles comprising ionizable lipid, Lipid 1 (LP1) or a comparable lipid. LNP-circular RNA construct was formulated with a N: P ratio of 4.5:1 and an ionizable lipid: DSPC helper lipid: Cholesterol: DMG-PEG (2000): ELA molar ratio of 38.25:7.65:37.4:1.7:15. As a control comparison, other circular RNAs were transfected using phosphate-buffered saline (PBS) instead of LNPs. The transfected circular RNAs were injected into mice (female mdx mice, 6-8 weeks, weight 18-22 g) intramuscularly into a single quadricep of each mouse. Both quadriceps of the mice were harvested 48 hours following transfection of the circular RNA and the tissues were analyzed using immunoprecipitation (IP). IP was conducted using V5 tag pull-down with anti-V5 tag visualization or V5-tag pull-down with anti-dystrophin visualization.

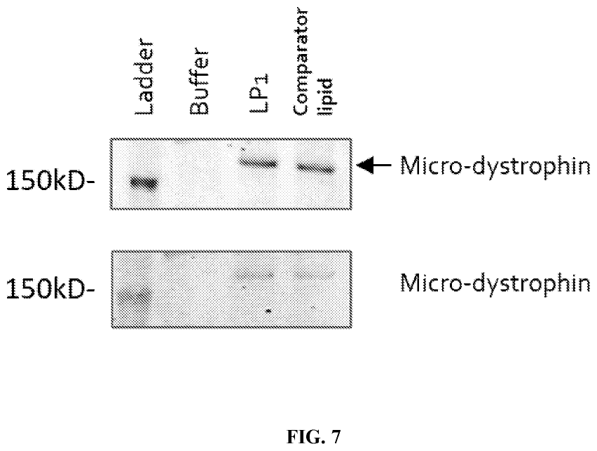

For both visualization techniques, both formulations of lipids comprising the circular RNA showed expression of micro-dystrophin (predicted mol. weight at 167 kD) as illustrated in FIG. 7.

| | | | | | | | Ionizable lipid:Helper |
|---|---|---|---|---|---|---|---|
| | Protein | Ionizable | | N:P | Helper | | Lipid:Cholesterol:PEG- |
| Group | Encoded | Lipid | Formulation | Ratio | Lipid | PEG-lipid | lipid (mol %):ELA |
| LP1-mini | Mini | LP1 | LP1 (4.5OB | 4.5:1 | DSPC | DMG-PEG | 38.25:7.65:37.4:1.7:15 |
| | dystrophin | | Neutral) | | | (2000) | |
| Comparator | Mini | Comparator | CP1 (5.7OA | 5.7:1 | DSPC | DMG-PEG | 42.5:8.5:32.725:1.275:15 |
| lipid-mini | dystrophin | lipid | Acidic) | | | (2000) | |

### Example 10

In vivo protein expression in mdx mice following intravenous injection of LNP formulated circular RNA encoding micro-dystrophin.

Engineered circular RNA encoding micro-dystrophin was encapsulated into a lipid nanoparticle comprising ionizable lipid, Lipid 1 (LP1). LNP-circular RNA construct was formulated with a N: P ratio of 4.5:1 and an ionizable lipid: DSPC helper lipid: Cholesterol: DMG-PEG (2000): ELA molar ratio of 38.25:7.65:37.4:1.7:15. As a control comparison, a buffer was used to transfect circular RNAs encoding micro-dystrophin. The circular RNA solutions were intravenously administered into mdx mice (female, 6-8 weeks old, weighted at 18-22g) via tail vein injection. 1 week following dosing, the tibialis anterior muscle of each mouse was harvested and analyzed using immunoprecipitation. Immunoprecipitation was conducted using V5 antibody.

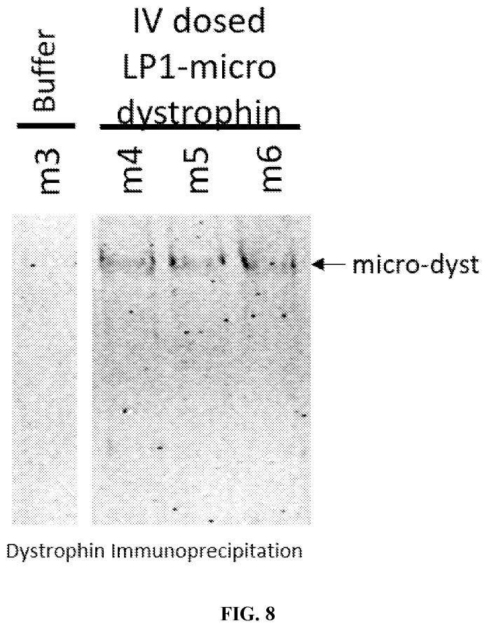

Each of the mice administered lipid nanoparticle-formulated engineered circular RNA intravenously showed expression of micro-dystrophin in vivo as illustrated in FIG. 8.

### Example 11

In vivo protein expression and localization in sarcolemma mdx mice following intravenous injection of LNP formulated with circular RNA encoding micro-dystrophin.

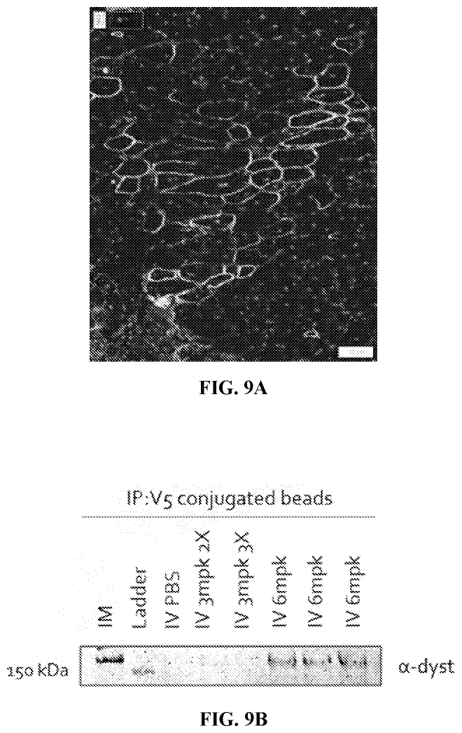

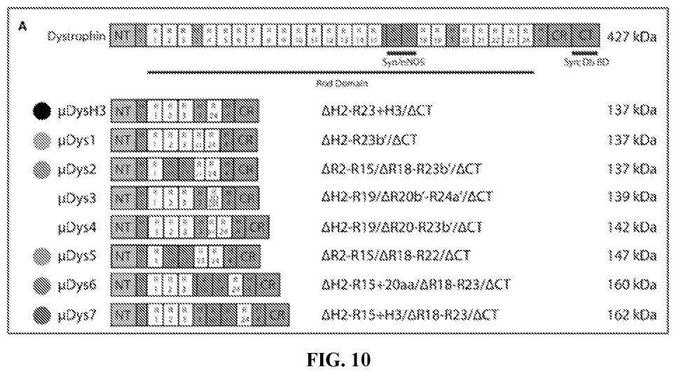

Engineered circular RNA encoding micro-dystrophin was encapsulated into a lipid nanoparticle comprising ionizable lipid, Lipid 1 (LP1). LNP-circular RNA construct was formulated with a N: P ratio of 4.5:1 and an ionizable lipid: DSPC helper lipid: Cholesterol: DMG-PEG (2000): ELA molar ratio of 38.25:7.65:37.4:1.7:15. As a control comparison, mice were treated with circular RNA encoding micro-dystrophin in buffer alone. Circular RNA solutions were intravenously administered to Mdx mice (female, 6-8 weeks old, weighed 18-22 g) by tail vein injection at 3 mpk (at either 2 or 3 dosages) or 6 mpk. Following dosing of the circular RNA solution, the tibialis anterior muscle of each mouse was harvested and analyzed using immunofluorescence. Immunofluorescence was conducted using a V5 tag pull down and anti-dystrophin visualization technique. The cross section of the tibialis anterior muscle showed micro-dystrophin localization to the sarcolemma of the transfected cells following three dosages of 3 mpk IV injection (FIG. 9A). Immunoprecipitation analysis of micro-dystrophin in tibialis anterior muscle from 6 mpk dosing is represented in FIG. 9B.

### Example 12

Circular RNA encoding micro-dystrophin does not localize in the liver post systemic injection.

Circular RNA encoding micro-dystrophin and encapsulated in LNP was injected into mice at various dosage regimes (1 dose at 6 mpk, 2 dosages at 3mpk, and 3 dosages at 3 mpk). The LNP used herein comprised ionizable lipid, Lipid 1 (LP1). LNP-circular RNA construct was formulated with a N: P ratio of 4.5:1 and an ionizable lipid: DSPC helper lipid: Cholesterol: DMG-PEG (2000): ELA molar ratio of 38.25:7.65:37.4:1.7:15. For comparison purposes, control animals were dosed using a phosphate-buffered saline (PBS) solution. At 7 days post injection, a Western blot or immunoprecipitation analysis was conducted on mouse liver tissues. Anti-V5 antibody was used in the Western blot analysis, while anti-dystrophin antibody was used in the immunoprecipitation analysis.

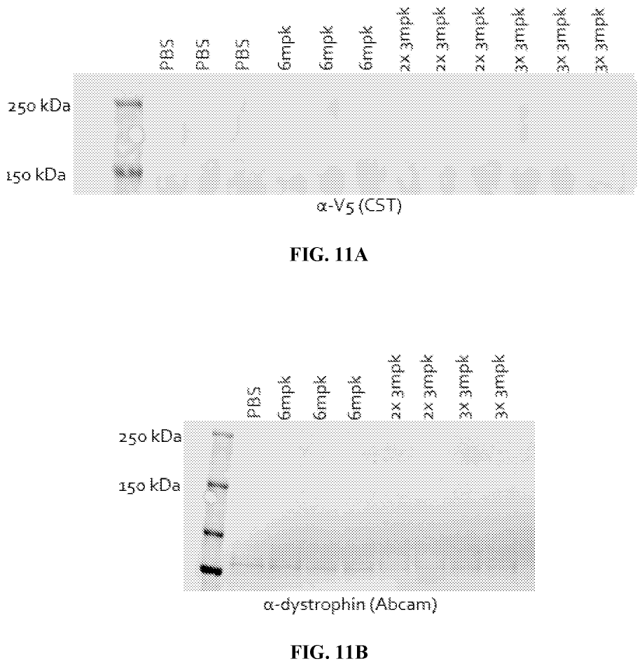

As shown in the Western blot analysis (FIG. 11A) and the immunoprecipitation analysis (FIG. 11B), micro-dystrophin was not present in the liver following systemic injection of circular RNA encoding micro-dystrophin. Thus, exemplifying that the circular RNA preparation did not produce micro-dystrophin in the liver following systemic injection at the aforementioned doses.

### Example 13

Example 13A: Expression of full-length dystrophin following transfection of circular RNA encoding full-length dystrophin in human skeletal muscle myotubes.

Circular RNA was engineered to encode V-5 antibody tagged full-length dystrophin (11841nt) and transfected into human skeletal muscle myotubes with Lipofectamine 3000 (Lipo) or Messenger Max (Mmax). Each of the circular RNA constructs transfected were dosed at 2750 ng, 5000 ng, 7500 ng or 10 μg and visualized by Western Blot analysis.

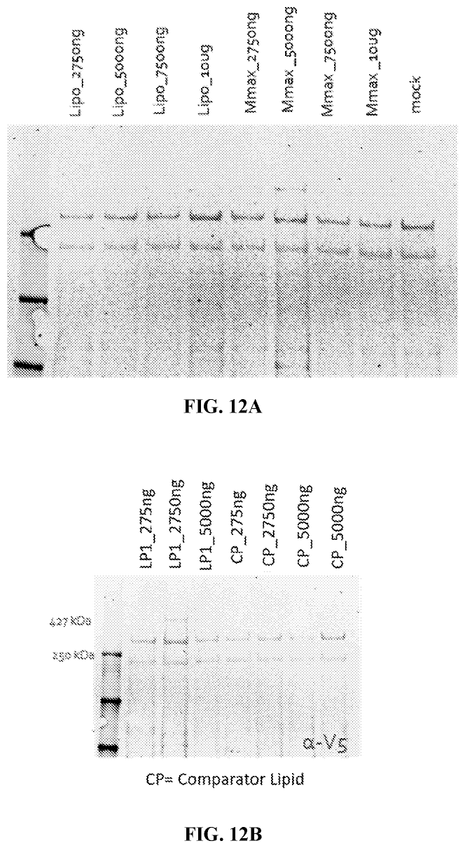

As illustrated in FIG. 12A, full-length dystrophin was produced at 427 kD in the Messenger Max transfected circular RNAs at a 7500 ng dosage.

Example 13B: Expression of full-length dystrophin following transfection of circular RNA encoding full-length dystrophin using lipid nanoparticles in human skeletal muscle myotubes.

Circular RNA was engineered to encode V-5 antibody tagged full-length dystrophin (11841nt) and transfected into human skeletal muscle myotubes using lipid nanoparticles comprising ionizable lipid, Lipid 1 or a comparator lipid. LNP-circular RNA construct was formulated with a N: P ratio of 4.5:1 and an ionizable lipid: DSPC helper lipid: Cholesterol: DMG-PEG (2000): ELA molar ratio of 38.25:7.65:37.4:1.7:15. Comparator lipid (CP) was formulated with a N: P ratio of 5.7:1 and an ionizable lipid: DSPC helper lipid: Cholesterol: DMG-PEG (2000): ELA molar ratio of 42.5:8.5:32.725:1.275:15. Each of the circular RNA constructs transfected were dosed at 275 ng, 2750 ng, or 5000 ng and analyzed using Western Blot.

As illustrated in FIG. 12B, full-length dystrophin was produced at 427 kD for LNP comprising Lipid 1 encapsulating circular RNAs at a 2750 ng dosage.

### Example 14

Protein expression in various tissue types following intravenous delivery of circular RNA-LNP.

Circular RNAs were engineered to encode for firefly luciferase and encapsulated in a lipid nanoparticle comprising ionizable lipid, Lipid 1 (LP1). LNP-circular RNA construct was formulated with a N: P ratio of 4.5:1 and an ionizable lipid: DSPC helper lipid: Cholesterol: DMG-PEG (2000): ELA molar ratio of 38.25:7.65:37.4:1.7:15.

The circular RNA-LNP constructs were intravenously administered to female C57BL/6J mice (n=5, 6-8 weeks old, weighted 18-22g). The mice were dosed at either 0.5, 1.0, 2.5, or 5.0 mpk. At 6 hours post-transfection, the organs of the mice were extracted and analyzed using ex vivo organ IVIS for total flux and percent expression in the kidneys, lungs, heart, left and right quadriceps, and left and right calf.

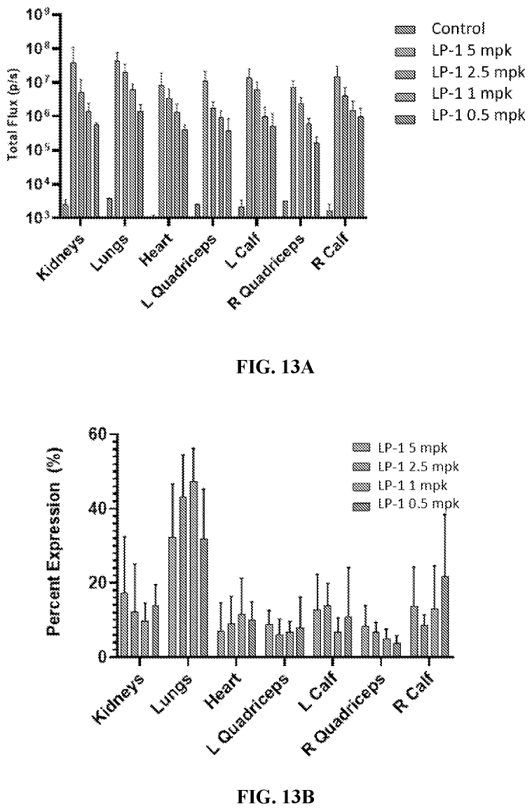

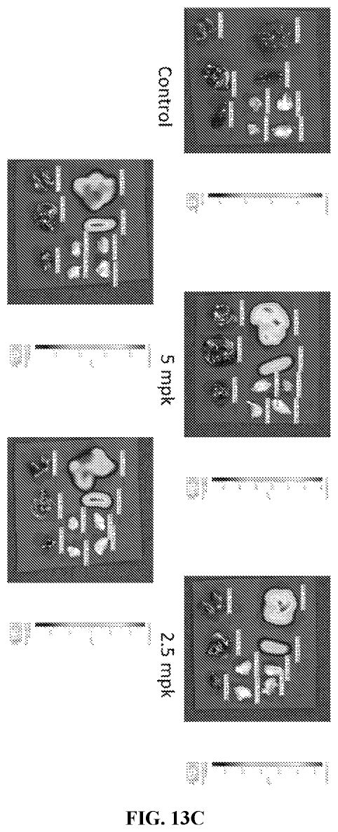

The circular RNA-LNP constructs were able to express the firefly luciferase at each of the dosages to the kidneys, lungs, heart, quadriceps, and calf (FIGS. 13A, 13B, and 13C).

### Example 15

Translation and protein expression of ORNA constructs encoding micro, Becker variant, or full-length dystrophin.

Engineered circular RNA encoding micro, Becker variant, or full-length dystrophin (ORNA constructs designed to be around 5 kb, 6.5 kb, or 12 kb respectively), were added to a HeLa Cell-Free translation (CFT) assay (HeLa 1-Step Human Coupled IVT Kit, ThermoFisher Scientific, Waltham, MA) containing HeLa cell accessory proteins capable of translating RNA into protein. The cell-free assay was allowed to run for 90 minutes at 30° C. for each of the dystrophin encoding circular RNA constructs. A Western Blot was then conducted 15 μL from the HeLa cell free assays to analyze the samples. The Western Bot was incubated with a V5 tag specific antibody (Cell Signaling, Danvers, MA) and later further incubated with a fluorescent antibody (Li-Cor, Lincoln, NE) for the Western Blot analysis.

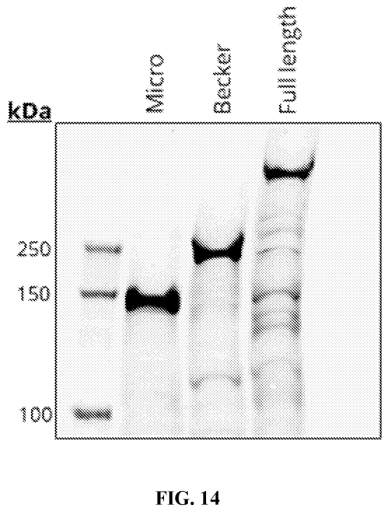

As seen in FIG. 14, micro dystrophin is present at 165 kDa (most left band), the Becker variant is present at 228 kDa (middle band) and the full-length dystrophin is present at 427 kDa (most right band) on the Western Blot analysis. These results show that the oRNA construct is able to translate and express micro, the Becker variant or full-length dystrophin.

### Example 16

Expression of a Becker variant, micro dystrophin, and full-length dystrophin in human primary skeletal muscle myotubes following translation of an oRNA

Example 16A: Myoblast Culture and Myotube Differentiation.

Primary human skeletal muscle (HSkM) cells (Promocell, Heidelberg, Germany) was prepared and plated at recommended seedling density of (3-5L per cm2) in SkGM-2 BulletKit Growth media and allowed to grow in a tissue culture incubator at 37° C. and 5% CO2 atmosphere. HskM cells were grown to 70-80% confluency in 0.1% gelatin (Sigma, St. Louis, MO) coated tissue culture plates. Once cells reached 70-80% confluency, growth media was removed, cells were washed twice in 1×PBS (Gibco, ThermoFisher Scientific) and changed to differentiation media consisting of F10 (1X) (Gibco, ThermoFisher Scientific) supplemented with 2% Horse Serum (Gibco, ThermoFisher Scientific) and 1% Pen Strep (Gibco, ThermoFisher Scientific). Media was changed daily for 5-6 days until nearly all myoblasts had fused to form myotubes.

Example 16B: Expression of Becker variant and micro dystrophin following transfection of L.NPs formulated with ORNA in human skeletal muscle myotubes.

Engineered circular RNA were designed to encode Becker variant dystrophin (containing about 46% internal deletion of a full-length dystrophin), micro dystrophin, or vinculin (positive control) and were formulated into lipid nanoparticles comprising Lipid 1 (see table below). Once myotubes were fully formed and ready for transfection, new differentiation media was added at 1 mL per 12-well plate, formulated LNPs (containing the engineered circular RNA) were then added directly into the differentiation media and cells are placed in a tissue culture incubator at 37° C. and 5% CO2 atmosphere for 48-hours prior to collection.

At 48-hours post transfection, differentiation media was removed, and cells were washed with 1×PBS (Gibco, ThermoFisher Scientific). 500 μL of trypsin (ReagentPack Lonza, Basel, Switzerland) were then added to the sample and kept at room temperature until cells detach. The trypsin was then quenched with 700 μL of trypsin neutralizing solution (ReagentPack Lonza, Basel Switzerland) and cells were collected and spun down. The supernatant (protein lysate) was removed, and cells were resuspended in RIPA buffer (ThermoFisher) supplemented with proteinase inhibitor cocktail (complete Sigma, Sigma-Aldrich, St. Louis, MO). Cells were vortexed, allowed to sit on ice for about 20 mins, and then spun down.

A Western Blot analysis was also conducted for the protein lysate samples (cell or tissue). Membranes were incubated with primary antibody (i.e., anti-V5 tag from abcam) overnight at 4° C. The next day, membrane was washed in 1X TBST and a secondary antibody (i.e., Goat anti-mouse from Invitrogen) was then added at 1:5000 for 1 hr at room temperature. Membranes were imaged using Odyssey CtX.

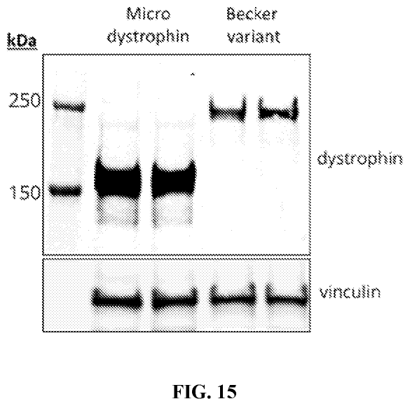

As seen in FIG. 15, the ORNA formulated within the LNPs were able to express micro dystrophin at 167 kDa and the Becker Variant at 228 kDa.

Example 16C: Expression of full-length dystrophin following lipofectamine transfection of ORNA in human skeletal muscle myotubes.

Engineered circular RNAs were designed to encode for full-length dystrophin protein and were transfected using lipofectamine. The lipofectamine transfection was prepared in a tube by adding Opti-MEM 50 μL to 1 μL of Messenger Max lipofectamine reagent and incubated at 10 mins at room temperature. In a separate tube, 50 μL optimum was added to either 1 μg or 2.75 μg of the engineered circular RNA encoding for full-length. 50 μL of the second tube was added to 50 μL of the first tube and incubated for 5 min at room temperature. Following incubation of the 100 μL sample, two 12-well plates were treated the sample containing the oRNA and lipofectamine solution.

At 48-hours post transfection, differentiation media was removed, and cells were washed with 1×PBS (Gibco, ThermoFisher Scientific). 500 μL of trypsin (ReagentPack Lonza, Basel, Switzerland) were then added to the sample and kept at room temperature until cells detach. The trypsin was then quenched with 700 μL of trypsin neutralizing solution (ReagentPack Lonza, Basel Switzerland) and cells were collected and spun down. The supernatant was removed, and cells were resuspended in RIPA buffer (ThermoFisher) supplemented with proteinase inhibitor cocktail (complete Sigma, Sigma-Aldrich, St. Louis, MO). Cells were vortexed, allowed to sit on ice for about 20 mins, and then spun down (the supernatant used was protein lysate).

Western Blot analysis was also conducted for the protein lysate samples. Membranes were incubated with primary antibody (i.e., anti-V5 tag from abcam) overnight at 4° C. The next day, membrane was washed in 1X TBST and a secondary antibody (i.e., Goat anti-mouse from Invitrogen) was then added at 1:5000 for 1 hr at room temperature. Membranes were imaged using Odyssey CtX.

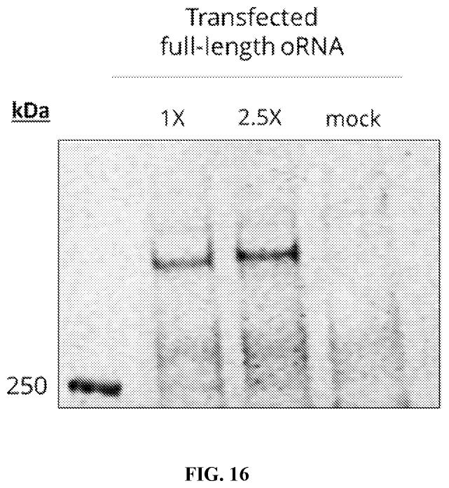

As seen in FIG. 16, the ORNA formulated within the LNPs were able to express full length dystrophin at 427 kDa.

| | | | | | | Ionizable lipid:Helper |
|---|---|---|---|---|---|---|
| Encoded | Ionizable | N:P | Structural | Helper | PEG- | Lipid:Cholesterol:PEG- |
| Protein | Lipid | Ratio | Lipid | Lipid | lipid | lipid:ELA (mol %) |
| Micro- | Lipid 1 | 4.5 | Cholesterol | DSPC | DMG- | 38.25:7.65:37.4:1.7:15 |
| dystrophin | | | | | PEG | |
| | | | | | 2000 | |
| Becker | Lipid 1 | 4.5 | Cholesterol | DSPC | DMG- | 38.25:7.65:37.4:1.7:15 |
| Variant | | | | | PEG | |
| Dystrophin | | | | | 2000 | |

### Example 17

Systemic delivery and expression of micro-dystrophin in mdx mouse muscle following systemic administration of LNPs formulated with ORNA encoding micro-dystrophin

Engineered circular RNA was designed to encode for micro-dystrophin and formulated into a lipid nanoparticle. LNPs were formulated with ionizable lipid 1 and comprised a molar ratio of ionizable lipid: DSPC Helper Lipid: Cholesterol: DMG-PEG 2000: Ethyl Lauroyl Arginate Hydrocholoride of 38.25:7.65:37.4:1.7:15 with a N: P ratio of 4.5. Mdx mice (aged 6-8 weeks) were then injected intravenously in the tail vein once at 12 mpk with the LNP-ORNA construct. Gastrocnemius muscles were collected 6-days post injection and flash frozen. Gastrocnemius tissue from an age matched mouse was collected and used to determine the percentage of micro dystrophin expression in the mdx mouse after intravenous injection.

Quadriceps muscles are collected 48 hr post injection and flash frozen in liquid nitrogen. For 1 whole quadriceps muscle, 2 mL of RIPA buffer (Thermo) supplemented with proteinase inhibitor cocktail (complete Sigma) was used for dissociation of the muscle tissue. The sample was then placed in the gentleMACS Octo Dissociator (Miltenyi Biotec). After dissociation, tubes were placed on ice for 5 mins and then centrifuged to collect supernatant. Protein lysate supernatant was then placed in a 1.5 mL tube and spun again.

For western blot analysis, protein lysate samples (cell or tissue) were made up at 1 μg/uL using NuPAGE 4X sample buffer (Thermo) and 1X NuPAGE reducing agent (Thermo). F Membranes were incubated with primary antibody (i.e., anti-dystrophin, abcam at 1:1000) overnight at 4° C. The next day, membrane was washed in 1X TBST, secondary antibody (i.e., goat anti-rabbit 800, Invitrogen) was then added at 1:5000 for 1 hr at room temperature. Membranes were imaged using Odyssey CtX.

Quadriceps muscle of the same mice were fresh frozen in OCT post-collection and cryosectioned into 10 micrometer sections onto a slide. Each section of the quad was stained using antibodies against Laminin-211 to outline the myofibers, micro dystrophin to detect protein expressed from intravenous LNP-ORNA injection and DAPI to outline the nuclei of individual cells.

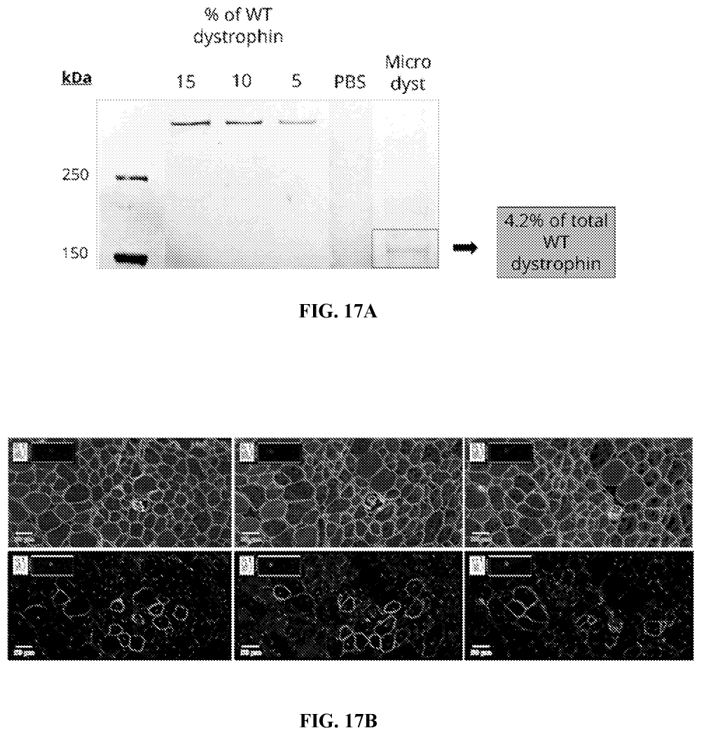

As seen in FIG. 17A, the LNP formulated with oRNA encoding for micro-dystrophin was able to express micro-dystrophin protein in gastrocnemius muscles of mdx mice following intravenous injection. Expression from one IV dose was determined to be 4.2%. Three sections of the quadriceps muscle of treated mdx mice are shown in FIG. 17B, sections were taken throughout the muscle to give a global view of micro-dystrophin expression distribution. The top panels have laminin-211 outlining the sarcolemma of the myofibers overlayed with micro-dystrophin (overlay in orange) with DAPI stain in blue showing individual nuclei. Bottom panels of the same figure are replicates of the top without the laminin-211 outline to give a better view of micro-dystrophin expression at the myofiber with DAPI outlining nuclei. The top panels of the figure show the Laminin-211 and micro-dystrophin overlay, showing correct localization of the muscle proteins expressed in the quadriceps following intravenous injection of the LNP-ORNA construct.

### Example 18

In vivo protein expression in mdx mice following intravenous injection of LNPs formulated with circular RNA encoding a Becker variant or micro-dystrophin.

Engineered circular RNAs encoding either a Becker variant or micro-dystrophin were encapsulated into lipid nanoparticles comprising ionizable lipid, Lipid 1 (LP1). The LNPs were formulated with a N: P ratio of 4.5:1 and an ionizable lipid: DSPC helper lipid: Cholesterol: DMG-PEG (2000): ELA molar ratio of 38.25:7.65:37.4:1.7:15. For controls, tissues from an animal injected with buffer (PBS) was used as a negative control (“M1”), lysates from cells expressing a Becker variant were used as a positive control for Becker variant expression, and lysates from tissues from an animal (“M12”) from a prior study injected intramuscularly with LNP formulated circular RNA encoding micro-dystrophin was used as a positive control for micro-dystrophin expression.

The LNPs were intravenously administered into mdx mice (female, 6-8 weeks old, weighing 18-22g) via tail vein injection. One day following dosing, tissues were collected and the diaphragm muscle of each mouse was analyzed using immunoprecipitation followed by western blotting. Immunoprecipitation was conducted using V5 antibody beads, and the blot was probed with an anti-dystrophin antibody that recognizes the Becker variant and micro-dystrophin. Certain mice administered lipid nanoparticle-formulated engineered circular RNA intravenously showed expression of the Becker variant (“M8” and “M9”) or micro-dystrophin (“M18” and “M19”) in vivo as illustrated in FIG. 18.

### EQUIVALENTS/OTHER EMBODIMENTS

Those skilled in the art will recognize or be able to ascertain using no more than routine experimentation, many equivalents to the specific embodiments described herein. Such equivalents are intended to be encompassed by the following claims.

TABLE 1
Dystrophin Constructs

| Name | Variant | Accession # | Sequence (bp) | Sequence (aa) |
|---|---|---|---|---|
| Full | Wild Type | NP_003997 | ATGCTTTGGTGGGAAGAAGTAGAGGACTGTTATGAAAGAGAAGATGT | MLWWEEVEDCYERED |
| Length | | | TCAAAAGAAAACATTCACAAAATGGGTAAATGCACAATTTTCTAAGT | VQKKTFTKWVNAQFS |
| human | | | TTGGGAAGCAGCATATTGAGAACCTCTTCAGTGACCTACAGGATGGG | KFGKQHIENLFSDLQD |
| dystrophin | | | AGGCGCCTCCTAGACCTCCTCGAAGGCCTGACAGGGCAAAAACTGCC | GRRLLDLLEGLTGQKL |
| | | | AAAAGAAAAAGGATCCACAAGAGTTCATGCCCTGAACAATGTCAACA | PKEKGSTRVHALNNVN |
| | | | AGGCACTGCGGGTTTTGCAGAACAATAATGTTGATTTAGTGAATATTG | KALRVLQNNNVDLVNI |
| | | | GAAGTACTGACATCGTAGATGGAAATCATAAACTGACTCTTGGTTTG | GSTDIVDGNHKLTLGLI |
| | | | ATTTGGAATATAATCCTCCACTGGCAGGTCAAAAATGTAATGAAAAA | WNIILHWQVKNVMKN |
| | | | TATCATGGCTGGATTGCAACAAACCAACAGTGAAAAGATTCTCCTGA | IMAGLQQTNSEKILLS |
| | | | GCTGGGTCCGACAATCAACTCGTAATTATCCACAGGTTAATGTAATCA | WVRQSTRNYPQVNVIN |
| | | | ACTTCACCACCAGCTGGTCTGATGGCCTGGCTTTGAATGCTCTCATCC | FTTSWSDGLALNALIH |
| | | | ATAGTCATAGGCCAGACCTATTTGACTGGAATAGTGTGGTTTGCCAGC | SHRPDLFDWNSVVCQ |
| | | | AGTCAGCCACACAACGACTGGAACATGCATTCAACATCGCCAGATAT | QSATQRLEHAFNIARY |
| | | | CAATTAGGCATAGAGAAACTACTCGATCCTGAAGATGTTGATACCAC | QLGIEKLLDPEDVDTT |
| | | | CTATCCAGATAAGAAGTCCATCTTAATGTACATCACATCACTCTTCCA | YPDKKSILMYITSLFQV |
| | | | AGTTTTGCCTCAACAAGTGAGCATTGAAGCCATCCAGGAAGTGGAAA | LPQQVSIEAIQEVEMLP |
| | | | TGTTGCCAAGGCCACCTAAAGTGACTAAAGAAGAACATTTTCAGTTA | RPPKVTKEEHFQLHHQ |
| | | | CATCATCAAATGCACTATTCTCAACAGATCACGGTCAGTCTAGCACAG | MHYSQQITVSLAQGYE |
| | | | GGATATGAGAGAACTTCTTCCCCTAAGCCTCGATTCAAGAGCTATGCC | RTSSPKPRFKSYAYTQ |
| | | | TACACACAGGCTGCTTATGTCACCACCTCTGACCCTACACGGAGCCCA | AAYVTTSDPTRSPFPSQ |
| | | | TTTCCTTCACAGCATTTGGAAGCTCCTGAAGACAAGTCATTTGGCAGT | HLEAPEDKSFGSSLME |
| | | | TCATTGATGGAGAGTGAAGTAAACCTGGACCGTTATCAAACAGCTTT | SEVNLDRYQTALEEVL |
| | | | AGAAGAAGTATTATCGTGGCTTCTTTCTGCTGAGGACACATTGCAAGC | SWLLSAEDTLQAQGEI |
| | | | ACAAGGAGAGATTTCTAATGATGTGGAAGTGGTGAAAGACCAGTTTC | SNDVEVVKDQFHTHE |
| | | | ATACTCATGAGGGGTACATGATGGATTTGACAGCCCATCAGGGCCGG | GYMMDLTAHQGRVG |
| | | | GTTGGTAATATTCTACAATTGGGAAGTAAGCTGATTGGAACAGGAAA | NILQLGSKLIGTGKLSE |
| | | | ATTATCAGAAGATGAAGAAACTGAAGTACAAGAGCAGATGAATCTCC | DEETEVQEQMNLLNSR |
| | | | TAAATTCAAGATGGGAATGCCTCAGGGTAGCTAGCATGGAAAAACAA | WECLRVASMEKQSNL |
| | | | AGCAATTTACATAGAGTTTTAATGGATCTCCAGAATCAGAAACTGAA | HRVLMDLQNQKLKEL |
| | | | AGAGTTGAATGACTGGCTAACAAAAACAGAAGAAAGAACAAGGAAA | NDWLTKTEERTRKME |
| | | | ATGGAGGAAGAGCCTCTTGGACCTGATCTTGAAGACCTAAAACGCCA | EEPLGPDLEDLKRQVQ |
| | | | AGTACAACAACATAAGGTGCTTCAAGAAGATCTAGAACAAGAACAA | QHKVLQEDLEQEQVR |
| | | | GTCAGGGTCAATTCTCTCACTCACATGGTGGTGGTAGTTGATGAATCT | VNSLTHMVVVVDESS |
| | | | AGTGGAGATCACGCAACTGCTGCTTTGGAAGAACAACTTAAGGTATT | GDHATAALEEQLKVL |
| | | | GGGAGATCGATGGGCAAACATCTGTAGATGGACAGAAGACCGCTGG | GDRWANICRWTEDRW |
| | | | GTTCTTTTACAAGACATCCTTCTCAAATGGCAACGTCTTACTGAAGAA | VLLQDILLKWQRLTEE |
| | | | CAGTGCCTTTTTAGTGCATGGCTTTCAGAAAAAGAAGATGCAGTGAA | QCLFSAWLSEKEDAVN |
| | | | CAAGATTCACACAACTGGCTTTAAAGATCAAAATGAAATGTTATCAA | KIHTTGFKDQNEMLSS |
| | | | GTCTTCAAAAACTGGCCGTTTTAAAAGCGGATCTAGAAAAGAAAAAG | LQKLAVLKADLEKKK |
| | | | CAATCCATGGGCAAACTGTATTCACTCAAACAAGATCTTCTTTCAACA | QSMGKLYSLKQDLLST |
| | | | CTGAAGAATAAGTCAGTGACCCAGAAGACGGAAGCATGGCTGGATA | LKNKSVTQKTEAWLD |
| | | | ACTTTGCCCGGTGTTGGGATAATTTAGTCCAAAAACTTGAAAAGAGT | NFARCWDNLVQKLEK |
| | | | ACAGCACAGATTTCACAGGCTGTCACCACCACTCAGCCATCACTAAC | STAQISQAVTTTQPSLT |
| | | | ACAGACAACTGTAATGGAAACAGTAACTACGGTGACCACAAGGGAA | QTTVMETVTTVTTREQ |
| | | | CAGATCCTGGTAAAGCATGCTCAAGAGGAACTTCCACCACCACCTCC | ILVKHAQEELPPPPPQK |
| | | | CCAAAAGAAGAGGCAGATTACTGTGGATTCTGAAATTAGGAAAAGGT | KRQITVDSEIRKRLDVD |
| | | | TGGATGTTGATATAACTGAACTTCACAGCTGGATTACTCGCTCAGAAG | ITELHSWITRSEAVLQS |
| | | | CTGTGTTGCAGAGTCCTGAATTTGCAATCTTTCGGAAGGAAGGCAACT | PEFAIFRKEGNFSDLKE |
| | | | TCTCAGACTTAAAAGAAAAAGTCAATGCCATAGAGCGAGAAAAAGCT | KVNAIEREKAEKFRKL |
| | | | GAGAAGTTCAGAAAACTGCAAGATGCCAGCAGATCAGCTCAGGCCCT | QDASRSAQALVEQMV |
| | | | GGTGGAACAGATGGTGAATGAGGGTGTTAATGCAGATAGCATCAAAC | NEGVNADSIKQASEQL |
| | | | AAGCCTCAGAACAACTGAACAGCCGGTGGATCGAATTCTGCCAGTTG | NSRWIEFCQLLSERLN |
| | | | CTAAGTGAGAGACTTAACTGGCTGGAGTATCAGAACAACATCATCGC | WLEYQNNIIAFYNQLQ |
| | | | TTTCTATAATCAGCTACAACAATTGGAGCAGATGACAACTACTGCTGA | QLEQMTTTAENWLKIQ |
| | | | AAACTGGTTGAAAATCCAACCCACCACCCCATCAGAGCCAACAGCAA | PTTPSEPTAIKSQLKICK |
| | | | TTAAAAGTCAGTTAAAAATTTGTAAGGATGAAGTCAACCGGCTATCA | DEVNRLSDLQPQIERL |
| | | | GATCTTCAACCTCAAATTGAACGATTAAAAATTCAAAGCATAGCCCT | KIQSIALKEKGQGPMFL |
| | | | GAAAGAGAAAGGACAAGGACCCATGTTCCTGGATGCAGACTTTGTGG | DADFVAFTNHFKQVFS |
| | | | CCTTTACAAATCATTTTAAGCAAGTCTTTTCTGATGTGCAGGCCAGAG | DVQAREKELQTIFDTLP |
| | | | AGAAAGAGCTACAGACAATTTTTGACACTTTGCCACCAATGCGCTATC | PMRYQETMSAIRTWV |
| | | | AGGAGACCATGAGTGCCATCAGGACATGGGTCCAGCAGTCAGAAACC | QQSETKLSIPQLSVTDY |
| | | | AAACTCTCCATACCTCAACTTAGTGTCACCGACTATGAAATCATGGAG | EIMEQRLGELQALQSS |
| | | | CAGAGACTCGGGGAATTGCAGGCTTTACAAAGTTCTCTGCAAGAGCA | LQEQQSGLYYLSTTVK |
| | | | ACAAAGTGGCCTATACTATCTCAGCACCACTGTGAAAGAGATGTCGA | EMSKKAPSEISRKYQSE |
| | | | AGAAAGCGCCCTCTGAAATTAGCCGGAAATATCAATCAGAATTTGAA | FEEIEGRWKKLSSQLV |
| | | | GAAATTGAGGGACGCTGGAAGAAGCTCTCCTCCCAGCTGGTTGAGCA | EHCQKLEEQMNKLRKI |
| | | | TTGTCAAAAGCTAGAGGAGCAAATGAATAAACTCCGAAAAATTCAGA | QNHIQTLKKWMAEVD |
| | | | ATCACATACAAACCCTGAAGAAATGGATGGCTGAAGTTGATGTTTTTC | VFLKEEWPALGDSEIL |
| | | | TGAAGGAGGAATGGCCTGCCCTTGGGGATTCAGAAATTCTAAAAAAG | KKQLKQCRLLVSDIQTI |
| | | | CAGCTGAAACAGTGCAGACTTTTAGTCAGTGATATTCAGACAATTCA | QPSLNSVNEGGQKIKN |
| | | | GCCCAGTCTAAACAGTGTCAATGAAGGTGGGCAGAAGATAAAGAAT | EAEPEFASRLETELKEL |
| | | | GAAGCAGAGCCAGAGTTTGCTTCGAGACTTGAGACAGAACTCAAAGA | NTQWDHMCQQVYAR |
| | | | ACTTAACACTCAGTGGGATCACATGTGCCAACAGGTCTATGCCAGAA | KEALKGGLEKTVSLQK |
| | | | AGGAGGCCTTGAAGGGAGGTTTGGAGAAAACTGTAAGCCTCCAGAA | DLSEMHEWMTQAEEE |
| | | | AGATCTATCAGAGATGCACGAATGGATGACACAAGCTGAAGAAGAG | YLERDFEYKTPDELQK |
| | | | TATCTTGAGAGAGATTTTGAATATAAAACTCCAGATGAATTACAGAA | AVEEMKRAKEEAQQK |
| | | | AGCAGTTGAAGAGATGAAGAGAGCTAAAGAAGAGGCCCAACAAAAA | EAKVKLLTESVNSVIA |
| | | | GAAGCGAAAGTGAAACTCCTTACTGAGTCTGTAAATAGTGTCATAGC | QAPPVAQEALKKELET |
| | | | TCAAGCTCCACCTGTAGCACAAGAGGCCTTAAAAAAGGAACTTGAAA | LTTNYQWLCTRLNGK |
| | | | CTCTAACCACCAACTACCAGTGGCTCTGCACTAGGCTGAATGGGAAA | CKTLEEVWACWHELL |
| | | | TGCAAGACTTTGGAAGAAGTTTGGGCATGTTGGCATGAGTTATTGTCA | SYLEKANKWLNEVEF |
| | | | TACTTGGAGAAAGCAAACAAGTGGCTAAATGAAGTAGAATTTAAACT | KLKTTENIPGGAEEISE |
| | | | TAAAACCACTGAAAACATTCCTGGCGGAGCTGAGGAAATCTCTGAGG | VLDSLENLMRHSEDNP |
| | | | TGCTAGATTCACTTGAAAATTTGATGCGACATTCAGAGGATAACCCA | NQIRILAQTLTDGGVM |
| | | | AATCAGATTCGCATATTGGCACAGACCCTAACAGATGGCGGAGTCAT | DELINEELETFNSRWRE |
| | | | GGATGAGCTAATCAATGAGGAACTTGAGACATTTAATTCTCGTTGGA | LHEEAVRRQKLLEQSI |
| | | | GGGAACTACATGAAGAGGCTGTAAGGAGGCAAAAGTTGCTTGAACA | QSAQETEKSLHLIQESL |
| | | | GAGCATCCAGTCTGCCCAGGAGACTGAAAAATCCTTACACTTAATCC | TFIDKQLAAYIADKVD |
| | | | AGGAGTCCCTCACATTCATTGACAAGCAGTTGGCAGCTTATATTGCAG | AAQMPQEAQKIQSDLT |
| | | | ACAAGGTGGACGCAGCTCAAATGCCTCAGGAAGCCCAGAAAATCCA | SHEISLEEMKKHNQGK |
| | | | ATCTGATTTGACAAGTCATGAGATCAGTTTAGAAGAAATGAAGAAAC | EAAQRVLSQIDVAQKK |
| | | | ATAATCAGGGGAAGGAGGCTGCCCAAAGAGTCCTGTCTCAGATTGAT | LQDVSMKFRLFQKPAN |
| | | | GTTGCACAGAAAAAATTACAAGATGTCTCCATGAAGTTTCGATTATTC | FEQRLQESKMILDEVK |
| | | | CAGAAACCAGCCAATTTTGAGCAGCGTCTACAAGAAAGTAAGATGAT | MHLPALETKSVEQEVV |
| | | | TTTAGATGAAGTGAAGATGCACTTGCCTGCATTGGAAACAAAGAGTG | QSQLNHCVNLYKSLSE |
| | | | TGGAACAGGAAGTAGTACAGTCACAGCTAAATCATTGTGTGAACTTG | VKSEVEMVIKTGRQIV |
| | | | TATAAAAGTCTGAGTGAAGTGAAGTCTGAAGTGGAAATGGTGATAAA | QKKQTENPKELDERVT |
| | | | GACTGGACGTCAGATTGTACAGAAAAAGCAGACGGAAAATCCCAAA | ALKLHYNELGAKVTER |
| | | | GAACTTGATGAAAGAGTAACAGCTTTGAAATTGCATTATAATGAGCT | KQQLEKCLKLSRKMR |
| | | | GGGAGCAAAGGTAACAGAAAGAAAGCAACAGTTGGAGAAATGCTTG | KEMNVLTEWLAATDM |
| | | | AAATTGTCCCGTAAGATGCGAAAGGAAATGAATGTCTTGACAGAATG | ELTKRSAVEGMPSNLD |
| | | | GCTGGCAGCTACAGATATGGAATTGACAAAGAGATCAGCAGTTGAAG | SEVAWGKATQKEIEKQ |
| | | | GAATGCCTAGTAATTTGGATTCTGAAGTTGCCTGGGGAAAGGCTACTC | KVHLKSITEVGEALKT |
| | | | AAAAAGAGATTGAGAAACAGAAGGTGCACCTGAAGAGTATCACAGA | VLGKKETLVEDKLSLL |
| | | | GGTAGGAGAGGCCTTGAAAACAGTTTTGGGCAAGAAGGAGACGTTG | NSNWIAVTSRAEEWLN |
| | | | GTGGAAGATAAACTCAGTCTTCTGAATAGTAACTGGATAGCTGTCAC | LLLEYQKHMETFDQN |
| | | | CTCCCGAGCAGAAGAGTGGTTAAATCTTTTGTTGGAATACCAGAAAC | VDHITKWIIQADTLLDE |
| | | | ACATGGAAACTTTTGACCAGAATGTGGACCACATCACAAAGTGGATC | SEKKKPQQKEDVLKRL |
| | | | ATTCAGGCTGACACACTTTTGGATGAATCAGAGAAAAAGAAACCCCA | KAELNDIRPKVDSTRD |
| | | | GCAAAAAGAAGACGTGCTTAAGCGTTTAAAGGCAGAACTGAATGAC | QAANLMANRGDHCRK |
| | | | ATACGCCCAAAGGTGGACTCTACACGTGACCAAGCAGCAAACTTGAT | LVEPQISELNHRFAAIS |
| | | | GGCAAACCGCGGTGACCACTGCAGGAAATTAGTAGAGCCCCAAATCT | HRIKTGKASIPLKELEQ |
| | | | CAGAGCTCAACCATCGATTTGCAGCCATTTCACACAGAATTAAGACT | FNSDIQKLLEPLEAEIQ |
| | | | GGAAAGGCCTCCATTCCTTTGAAGGAATTGGAGCAGTTTAACTCAGA | QGVNLKEEDFNKDMN |
| | | | TATACAAAAATTGCTTGAACCACTGGAGGCTGAAATTCAGCAGGGGG | EDNEGTVKELLQRGDN |
| | | | TGAATCTGAAAGAGGAAGACTTCAATAAAGATATGAATGAAGACAAT | LQQRITDERKREEIKIK |
| | | | GAGGGTACTGTAAAAGAATTGTTGCAAAGAGGAGACAACTTACAACA | QQLLQTKHNALKDLRS |
| | | | AAGAATCACAGATGAGAGAAAGCGAGAGGAAATAAAGATAAAACAG | QRRKKALEISHQWYQ |
| | | | CAGCTGTTACAGACAAAACATAATGCTCTCAAGGATTTGAGGTCTCA | YKRQADDLLKCLDDIE |
| | | | AAGAAGAAAAAAGGCTCTAGAAATTTCTCATCAGTGGTATCAGTACA | KKLASLPEPRDERKIKE |
| | | | AGAGGCAGGCTGATGATCTCCTGAAATGCTTGGATGACATTGAAAAA | IDRELQKKKEELNAVR |
| | | | AAATTAGCCAGCCTACCTGAGCCCAGAGATGAAAGGAAAATAAAGG | RQAEGLSEDGAAMAV |
| | | | AAATTGATCGGGAATTGCAGAAGAAGAAAGAGGAGCTGAATGCAGT | EPTQIQLSKRWREIESK |
| | | | GCGTAGGCAAGCTGAGGGCTTGTCTGAGGATGGGGCCGCAATGGCAG | FAQFRRLNFAQIHTVR |
| | | | TGGAGCCAACTCAGATCCAGCTCAGCAAGCGCTGGCGGGAAATTGAG | EETMMVMTEDMPLEIS |
| | | | AGCAAATTTGCTCAGTTTCGAAGACTCAACTTTGCACAAATTCACACT | YVPSTYLTEITHVSQAL |
| | | | GTCCGTGAAGAAACGATGATGGTGATGACTGAAGACATGCCTTTGGA | LEVEQLLNAPDLCAKD |
| | | | AATTTCTTATGTGCCTTCTACTTATTTGACTGAAATCACTCATGTCTCA | FEDLFKQEESLKNIKDS |
| | | | CAAGCCCTATTAGAAGTGGAACAACTTCTCAATGCTCCTGACCTCTGT | LQQSSGRIDIIHSKKTA |
| | | | GCTAAGGACTTTGAAGATCTCTTTAAGCAAGAGGAGTCTCTGAAGAA | ALQSATPVERVKLQEA |
| | | | TATAAAAGATAGTCTACAACAAAGCTCAGGTCGGATTGACATTATTC | LSQLDFQWEKVNKMY |
| | | | ATAGCAAGAAGACAGCAGCATTGCAAAGTGCAACGCCTGTGGAAAG | KDRQGRFDRSVEKWR |
| | | | GGTGAAGCTACAGGAAGCTCTCTCCCAGCTTGATTTCCAATGGGAAA | RFHYDIKIFNQWLTEA |
| | | | AAGTTAACAAAATGTACAAGGACCGACAAGGGCGATTTGACAGATCT | EQFLRKTQIPENWEHA |
| | | | GTTGAGAAATGGCGGCGTTTTCATTATGATATAAAGATATTTAATCAG | KYKWYLKELQDGIGQ |
| | | | TGGCTAACAGAAGCTGAACAGTTTCTCAGAAAGACACAAATTCCTGA | RQTVVRTLNATGEEIIQ |
| | | | GAATTGGGAACATGCTAAATACAAATGGTATCTTAAGGAACTCCAGG | QSSKTDASILQEKLGSL |
| | | | ATGGCATTGGGCAGCGGCAAACTGTTGTCAGAACATTGAATGCAACT | NLRWQEVCKQLSDRK |
| | | | GGGGAAGAAATAATTCAGCAATCCTCAAAAACAGATGCCAGTATTCT | KRLEEQKNILSEFQRDL |
| | | | ACAGGAAAAATTGGGAAGCCTGAATCTGCGGTGGCAGGAGGTCTGCA | NEFVLWLEEADNIASIP |
| | | | AACAGCTGTCAGACAGAAAAAAGAGGCTAGAAGAACAAAAGAATAT | LEPGKEQQLKEKLEQV |
| | | | CTTGTCAGAATTTCAAAGAGATTTAAATGAATTTGTTTTATGGTTGGA | KLLVEELPLRQGILKQL |
| | | | GGAAGCAGATAACATTGCTAGTATCCCACTTGAACCTGGAAAAGAGC | NETGGPVLVSAPISPEE |
| | | | AGCAACTAAAAGAAAAGCTTGAGCAAGTCAAGTTACTGGTGGAAGA | QDKLENKLKQTNLQWI |
| | | | GTTGCCCCTGCGCCAGGGAATTCTCAAACAATTAAATGAAACTGGAG | KVSRALPEKQGEIEAQI |
| | | | GACCCGTGCTTGTAAGTGCTCCCATAAGCCCAGAAGAGCAAGATAAA | KDLGQLEKKLEDLEEQ |
| | | | CTTGAAAATAAGCTCAAGCAGACAAATCTCCAGTGGATAAAGGTTTC | LNHLLLWLSPIRNQLEI |
| | | | CAGAGCTTTACCTGAGAAACAAGGAGAAATTGAAGCTCAAATAAAA | YNQPNQEGPFDVKETE |
| | | | GACCTTGGGCAGCTTGAAAAAAAGCTTGAAGACCTTGAAGAGCAGTT | IAVQAKQPDVEEILSKG |
| | | | AAATCATCTGCTGCTGTGGTTATCTCCTATTAGGAATCAGTTGGAAAT | QHLYKEKPATQPVKRK |
| | | | TTATAACCAACCAAACCAAGAAGGACCATTTGACGTTAAGGAAACTG | LEDLSSEWKAVNRLLQ |
| | | | AAATAGCAGTTCAAGCTAAACAACCGGATGTGGAAGAGATTTTGTCT | ELRAKQPDLAPGLTTIG |
| | | | AAAGGGCAGCATTTGTACAAGGAAAAACCAGCCACTCAGCCAGTGA | ASPTQTVTLVTQPVVT |
| | | | AGAGGAAGTTAGAAGATCTGAGCTCTGAGTGGAAGGCGGTAAACCGT | KETAISKLEMPSSLMLE |
| | | | TTACTTCAAGAGCTGAGGGCAAAGCAGCCTGACCTAGCTCCTGGACT | VPALADFNRAWTELTD |
| | | | GACCACTATTGGAGCCTCTCCTACTCAGACTGTTACTCTGGTGACACA | WLSLLDQVIKSQRVMV |
| | | | ACCTGTGGTTACTAAGGAAACTGCCATCTCCAAACTAGAAATGCCAT | GDLEDINEMIIKQKAT |
| | | | CTTCCTTGATGTTGGAGGTACCTGCTCTGGCAGATTTCAACCGGGCTT | MQDLEQRRPQLEELIT |
| | | | GGACAGAACTTACCGACTGGCTTTCTCTGCTTGATCAAGTTATAAAAT | AAQNLKNKTSNQEAR |
| | | | CACAGAGGGTGATGGTGGGTGACCTTGAGGATATCAACGAGATGATC | TIITDRIERIQNQWDEV |
| | | | ATCAAGCAGAAGGCAACAATGCAGGATTTGGAACAGAGGCGTCCCC | QEHLQNRRQQLNEML |
| | | | AGTTGGAAGAACTCATTACCGCTGCCCAAAATTTGAAAAACAAGACC | KDSTQWLEAKEEAEQ |
| | | | AGCAATCAAGAGGCTAGAACAATCATTACGGATCGAATTGAAAGAAT | VLGQARAKLESWKEG |
| | | | TCAGAATCAGTGGGATGAAGTACAAGAACACCTTCAGAACCGGAGGC | PYTVDAIQKKITETKQL |
| | | | AACAGTTGAATGAAATGTTAAAGGATTCAACACAATGGCTGGAAGCT | AKDLRQWQTNVDVAN |
| | | | AAGGAAGAAGCTGAGCAGGTCTTAGGACAGGCCAGAGCCAAGCTTG | DLALKLLRDYSADDTR |
| | | | AGTCATGGAAGGAGGGTCCCTATACAGTAGATGCAATCCAAAAGAAA | KVHMITENINASWRSI |
| | | | ATCACAGAAACCAAGCAGTTGGCCAAAGACCTCCGCCAGTGGCAGAC | HKRVSEREAALEETHR |
| | | | AAATGTAGATGTGGCAAATGACTTGGCCCTGAAACTTCTCCGGGATT | LLQQFPLDLEKFLAWL |
| | | | ATTCTGCAGATGATACCAGAAAAGTCCACATGATAACAGAGAATATC | TEAETTANVLQDATRK |
| | | | AATGCCTCTTGGAGAAGCATTCATAAAAGGGTGAGTGAGCGAGAGGC | ERLLEDSKGVKELMKQ |
| | | | TGCTTTGGAAGAAACTCATAGATTACTGCAACAGTTCCCCCTGGACCT | WQDLQGEIEAHTDVY |
| | | | GGAAAAGTTTCTTGCCTGGCTTACAGAAGCTGAAACAACTGCCAATG | HNLDENSQKILRSLEGS |
| | | | TCCTACAGGATGCTACCCGTAAGGAAAGGCTCCTAGAAGACTCCAAG | DDAVLLQRRLDNMNF |
| | | | GGAGTAAAAGAGCTGATGAAACAATGGCAAGACCTCCAAGGTGAAA | KWSELRKKSLNIRSHL |
| | | | TTGAAGCTCACACAGATGTTTATCACAACCTGGATGAAAACAGCCAA | EASSDQWKRLHLSLQE |
| | | | AAAATCCTGAGATCCCTGGAAGGTTCCGATGATGCAGTCCTGTTACA | LLVWLQLKDDELSRQ |
| | | | AAGACGTTTGGATAACATGAACTTCAAGTGGAGTGAACTTCGGAAAA | APIGGDFPAVQKQNDV |
| | | | AGTCTCTCAACATTAGGTCCCATTTGGAAGCCAGTTCTGACCAGTGGA | HRAFKRELKTKEPVIM |
| | | | AGCGTCTGCACCTTTCTCTGCAGGAACTTCTGGTGTGGCTACAGCTGA | STLETVRIFLTEQPLEG |
| | | | AAGATGATGAATTAAGCCGGCAGGCACCTATTGGAGGCGACTTTCCA | LEKLYQEPRELPPEERA |
| | | | GCAGTTCAGAAGCAGAACGATGTACATAGGGCCTTCAAGAGGGAATT | QNVTRLLRKQAEEVNT |
| | | | GAAAACTAAAGAACCTGTAATCATGAGTACTCTTGAGACTGTACGAA | EWEKLNLHSADWQRK |
| | | | TATTTCTGACAGAGCAGCCTTTGGAAGGACTAGAGAAACTCTACCAG | IDETLERLRELQEATDE |
| | | | GAGCCCAGAGAGCTGCCTCCTGAGGAGAGAGCCCAGAATGTCACTCG | LDLKLRQAEVIKGSWQ |
| | | | GCTTCTACGAAAGCAGGCTGAGGAGGTCAATACTGAGTGGGAAAAAT | PVGDLLIDSLQDHLEK |
| | | | TGAACCTGCACTCCGCTGACTGGCAGAGAAAAATAGATGAGACCCTT | VKALRGEIAPLKENVS |
| | | | GAAAGACTCCAGGAACTTCAAGAGGCCACGGATGAGCTGGACCTCAA | HVNDLARQLTTLGIQL |
| | | | GCTGCGCCAAGCTGAGGTGATCAAGGGATCCTGGCAGCCCGTGGGCG | SPYNLSTLEDLNTRWK |
| | | | ATCTCCTCATTGACTCTCTCCAAGATCACCTCGAGAAAGTCAAGGCAC | LLQVAVEDRVRQLHE |
| | | | TTCGAGGAGAAATTGCGCCTCTGAAAGAGAACGTGAGCCACGTCAAT | AHRDFGPASQHFLSTS |
| | | | GACCTTGCTCGCCAGCTTACCACTTTGGGCATTCAGCTCTCACCGTAT | VQGPWERAISPNKVPY |
| | | | AACCTCAGCACTCTGGAAGACCTGAACACCAGATGGAAGCTTCTGCA | YINHETQTTCWDHPK |
| | | | GGTGGCCGTCGAGGACCGAGTCAGGCAGCTGCATGAAGCCCACAGG | MTELYQSLADLNNVRF |
| | | | GACTTTGGTCCAGCATCTCAGCACTTTCTTTCCACGTCTGTCCAGGGTC | SAYRTAMKLRRLQKA |
| | | | CCTGGGAGAGAGCCATCTCGCCAAACAAAGTGCCCTACTATATCAAC | LCLDLLSLSAACDALD |
| | | | CACGAGACTCAAACAACTTGCTGGGACCATCCCAAAATGACAGAGCT | QHNLKQNDQPMDILQI |
| | | | CTACCAGTCTTTAGCTGACCTGAATAATGTCAGATTCTCAGCTTATAG | INCLTTIYDRLEQEHNN |
| | | | GACTGCCATGAAACTCCGAAGACTGCAGAAGGCCCTTTGCTTGGATC | LVNVPLCVDMCLNWL |
| | | | TCTTGAGCCTGTCAGCTGCATGTGATGCCTTGGACCAGCACAACCTCA | LNVYDTGRTGRIRVLS |
| | | | AGCAAAATGACCAGCCCATGGATATCCTGCAGATTATTAATTGTTTGA | FKTGIISLCKAHLEDKY |
| | | | CCACTATTTATGACCGCCTGGAGCAAGAGCACAACAATTTGGTCAAC | RYLFKQVASSTGFCDQ |
| | | | GTCCCTCTCTGCGTGGATATGTGTCTGAACTGGCTGCTGAATGTTTAT | RRLGLLLHDSIQIPRQL |
| | | | GATACGGGACGAACAGGGAGGATCCGTGTCCTGTCTTTTAAAACTGG | GEVASFGGSNIEPSVRS |
| | | | CATCATTTCCCTGTGTAAAGCACATTTGGAAGACAAGTACAGATACCT | CFQFANNKPEIEAALFL |
| | | | TTTCAAGCAAGTGGCAAGTTCAACAGGATTTTGTGACCAGCGCAGGC | DWMRLEPQSMVWLPV |
| | | | TGGGCCTCCTTCTGCATGATTCTATCCAAATTCCAAGACAGTTGGGTG | LHRVAAAETAKHQAK |
| | | | AAGTTGCATCCTTTGGGGGCAGTAACATTGAGCCAAGTGTCCGGAGC | CNICKECPIIGFRYRSLK |
| | | | TGCTTCCAATTTGCTAATAATAAGCCAGAGATCGAAGCGGCCCTCTTC | HFNYDICQSCFFSGRV |
| | | | CTAGACTGGATGAGACTGGAACCCCAGTCCATGGTGTGGCTGCCCGT | AKGHKMHYPMVEYCT |
| | | | CCTGCACAGAGTGGCTGCTGCAGAAACTGCCAAGCATCAGGCCAAAT | PTTSGEDVRDFAKVLK |
| | | | GTAACATCTGCAAAGAGTGTCCAATCATTGGATTCAGGTACAGGAGT | NKFRTKRYFAKHPRM |
| | | | CTAAAGCACTTTAATTATGACATCTGCCAAAGCTGCTTTTTTTCTGGTC | GYLPVQTVLEGDNME |
| | | | GAGTTGCAAAAGGCCATAAAATGCACTATCCCATGGTGGAATATTGC | TPVTLINFWPVDSAPAS |
| | | | ACTCCGACTACATCAGGAGAAGATGTTCGAGACTTTGCCAAGGTACT | SPQLSHDDTHSRIEHYA |
| | | | AAAAAACAAATTTCGAACCAAAAGGTATTTTGCGAAGCATCCCCGAA | SRLAEMENSNGSYLND |
| | | | TGGGCTACCTGCCAGTGCAGACTGTCTTAGAGGGGGACAACATGGAA | SISPNESIDDEHLLIQHY |
| | | | ACTCCCGTTACTCTGATCAACTTCTGGCCAGTAGATTCTGCGCCTGCC | CQSLNQDSPLSQPRSPA |
| | | | TCGTCCCCTCAGCTTTCACACGATGATACTCATTCACGCATTGAACAT | QILISLESEERGELERIL |
| | | | TATGCTAGCAGGCTAGCAGAAATGGAAAACAGCAATGGATCTTATCT | ADLEEENRNLQAEYDR |
| | | | AAATGATAGCATCTCTCCTAATGAGAGCATAGATGATGAACATTTGTT | LKQQHEHKGLSPLPSPP |
| | | | AATCCAGCATTACTGCCAAAGTTTGAACCAGGACTCCCCCCTGAGCC | EMMPTSPQSPRDAELI |
| | | | AGCCTCGTAGTCCTGCCCAGATCTTGATTTCCTTAGAGAGTGAGGAAA | AEAKLLRQHKGRLEAR |
| | | | GAGGGGAGCTAGAGAGAATCCTAGCAGATCTTGAGGAAGAAAACAG | MQILEDHNKQLESQLH |
| | | | GAATCTGCAAGCAGAATATGACCGTCTAAAGCAGCAGCACGAACATA | RLRQLLEQPQAEAKVN |
| | | | AAGGCCTGTCCCCACTGCCGTCCCCTCCTGAAATGATGCCCACCTCTC | GTTVSSPSTSLQRSDSS |
| | | | CCCAGAGTCCCCGGGATGCTGAGCTCATTGCTGAGGCCAAGCTACTG | QPMLLRVVGSQTSDSM |
| | | | CGTCAACACAAAGGCCGCCTGGAAGCCAGGATGCAAATCCTGGAAG | GEEDLLSPPQDTSTGLE |
| | | | ACCACAATAAACAGCTGGAGTCACAGTTACACAGGCTAAGGCAGCTG | EVMEQLNNSFPSSRGR |
| | | | CTGGAGCAACCCCAGGCAGAGGCCAAAGTGAATGGCACAACGGTGT | NTPGKPMREDTM (SEQ |
| | | | CCTCTCCTTCTACCTCTCTACAGAGGTCCGACAGCAGTCAGCCTATGC | ID NO: 2) |
| | | | TGCTCCGAGTGGTTGGCAGTCAAACTTCGGACTCCATGGGTGAGGAA | |
| | | | GATCTTCTCAGTCCTCCCCAGGACACAAGCACAGGGTTAGAGGAGGT | |
| | | | GATGGAGCAACTCAACAACTCCTTCCCTAGTTCAAGAGGAAGAAATA | |
| | | | CCCCTGGAAAGCCAATGAGAGAGGACACAATG (SEQ ID NO: 1) | |
| Full | Wild Type | NP_003997 | ATGCTTTGGTGGGAAGAAGTCGAGGACTGCTACGAGCGCGAGGACGT | MLWWEEVEDCYERED |
| Length | protein | | GCAGAAGAAAACCTTCACCAAATGGGTCAACGCCCAGTTCAGCAAGT | VQKKTFTKWVNAQFS |
| human | (CO | | TCGGCAAGCAGCACATCGAGAACCTGTTCAGCGACCTGCAGGATGGC | KFGKQHIENLFSDLQD |
| dystrophin | nucleotide | | AGAAGGCTGCTGGATCTGCTGGAAGGCCTGACAGGACAGAAGCTGCC | GRRLLDLLEGLTGQKL |
| | seq) | | CAAAGAGAAGGGCAGCACAAGAGTGCACGCCCTGAACAACGTGAAC | PKEKGSTRVHALNNVN |
| | | | AAGGCCCTGAGAGTGCTGCAGAACAACAACGTGGACCTGGTCAACAT | KALRVLQNNNVDLVNI |
| | | | CGGCAGCACCGACATCGTGGACGGCAACCACAAACTGACCCTGGGCC | GSTDIVDGNHKLTLGLI |
| | | | TGATCTGGAACATCATCCTGCACTGGCAAGTGAAGAACGTGATGAAG | WNIILHWQVKNVMKN |
| | | | AACATCATGGCCGGCCTGCAGCAGACCAACAGCGAGAAGATTCTGCT | IMAGLQQTNSEKILLS |
| | | | GAGCTGGGTCCGACAGAGCACCCGGAATTACCCTCAAGTGAACGTGA | WVRQSTRNYPQVNVIN |
| | | | TCAACTTCACCACCTCTTGGAGCGACGGACTGGCCCTGAATGCCCTGA | FTTSWSDGLALNALIH |
| | | | TCCACAGCCACAGACCTGACCTGTTCGACTGGAACAGCGTCGTGTGTC | SHRPDLFDWNSVVCQ |
| | | | AGCAGAGCGCCACACAGAGGCTGGAACACGCCTTCAATATCGCCAGA | QSATQRLEHAFNIARY |
| | | | TACCAGCTGGGCATCGAGAAACTGCTGGACCCCGAGGATGTGGACAC | QLGIEKLLDPEDVDTT |
| | | | CACCTATCCTGACAAGAAATCCATCCTCATGTACATCACCAGCCTGTT | YPDKKSILMYITSLFQV |
| | | | CCAGGTGCTGCCCCAGCAGGTTTCCATCGAGGCCATTCAAGAGGTCG | LPQQVSIEAIQEVEMLP |
| | | | AGATGCTGCCCAGACCTCCTAAAGTGACCAAAGAGGAACACTTCCAG | RPPKVTKEEHFQLHHQ |
| | | | CTGCACCACCAGATGCACTACTCTCAGCAGATCACCGTGTCTCTGGCC | MHYSQQITVSLAQGYE |
| | | | CAGGGCTACGAGAGAACAAGCAGCCCCAAGCCTCGGTTCAAGAGCTA | RTSSPKPRFKSYAYTQ |
| | | | CGCCTATACACAGGCCGCCTACGTGACCACCAGCGATCCTACAAGAA | AAYVTTSDPTRSPFPSQ |
| | | | GCCCATTTCCTAGCCAGCACCTCGAGGCCCCTGAGGATAAGAGCTTTG | HLEAPEDKSFGSSLME |
| | | | GCAGCAGCCTGATGGAAAGCGAAGTGAACCTGGACCGCTACCAGAC | SEVNLDRYQTALEEVL |
| | | | AGCCCTGGAAGAGGTTCTGAGCTGGCTGCTGTCTGCCGAGGATACAC | SWLLSAEDTLQAQGEI |
| | | | TGCAGGCTCAGGGCGAGATCAGCAACGACGTGGAAGTGGTCAAGGA | SNDVEVVKDQFHTHE |
| | | | CCAGTTTCACACCCACGAGGGCTACATGATGGACCTGACAGCCCACC | GYMMDLTAHQGRVG |
| | | | AGGGCAGAGTGGGCAATATTCTGCAGCTGGGCTCCAAGCTGATCGGC | NILQLGSKLIGTGKLSE |
| | | | ACAGGCAAGCTGAGCGAGGACGAAGAGACAGAGGTGCAAGAGCAGA | DEETEVQEQMNLLNSR |
| | | | TGAACCTGCTGAACAGCAGATGGGAGTGTCTGAGAGTGGCCAGCATG | WECLRVASMEKQSNL |
| | | | GAAAAGCAGAGCAACCTGCACCGGGTGCTGATGGATCTCCAGAACCA | HRVLMDLQNQKLKEL |
| | | | GAAGCTGAAAGAGCTGAACGACTGGCTGACCAAGACCGAGGAACGG | NDWLTKTEERTRKME |
| | | | ACCCGGAAGATGGAAGAGGAACCTCTGGGACCCGACCTGGAAGATCT | EEPLGPDLEDLKRQVQ |
| | | | GAAAAGACAGGTGCAGCAGCATAAGGTGCTGCAAGAGGACCTCGAG | QHKVLQEDLEQEQVR |
| | | | CAAGAGCAAGTGCGCGTGAACAGCCTGACACACATGGTGGTGGTCGT | VNSLTHMVVVVDESS |
| | | | GGATGAGAGCAGCGGAGATCATGCCACAGCCGCTCTGGAAGAACAG | GDHATAALEEQLKVL |
| | | | CTGAAGGTGCTGGGAGACAGATGGGCCAATATCTGCCGGTGGACCGA | GDRWANICRWTEDRW |
| | | | GGATAGATGGGTGCTGCTCCAGGACATCCTGCTGAAGTGGCAGCGGC | VLLQDILLKWQRLTEE |
| | | | TGACAGAGGAACAGTGCCTGTTTAGCGCCTGGCTGTCCGAGAAAGAG | QCLFSAWLSEKEDAVN |
| | | | GACGCCGTCAACAAGATCCACACCACCGGCTTCAAGGATCAGAATGA | KIHTTGFKDQNEMLSS |
| | | | GATGCTGAGCAGCCTGCAGAAACTGGCCGTGCTGAAGGCTGACCTGG | LQKLAVLKADLEKKK |
| | | | AAAAGAAAAAGCAGTCCATGGGCAAGCTGTACTCCCTGAAGCAGGA | QSMGKLYSLKQDLLST |
| | | | CCTGCTGAGCACACTGAAGAACAAGAGCGTGACCCAGAAAACCGAG | LKNKSVTQKTEAWLD |
| | | | GCCTGGCTGGACAACTTCGCCAGATGCTGGGACAACCTGGTGCAGAA | NFARCWDNLVQKLEK |
| | | | GCTGGAAAAGTCCACCGCTCAGATCTCTCAGGCCGTGACCACAACAC | STAQISQAVTTTQPSLT |
| | | | AGCCTTCTCTGACCCAGACCACCGTGATGGAAACAGTGACCACAGTG | QTTVMETVTTVTTREQ |
| | | | ACAACCCGCGAGCAGATCCTGGTCAAGCACGCCCAAGAAGAACTGCC | ILVKHAQEELPPPPPQK |
| | | | TCCTCCACCTCCTCAGAAGAAACGGCAGATCACAGTGGACAGCGAGA | KRQITVDSEIRKRLDVD |
| | | | TCCGGAAGAGACTGGACGTGGACATCACCGAGCTGCACAGCTGGATC | ITELHSWITRSEAVLQS |
| | | | ACCAGATCCGAAGCCGTGCTGCAGTCCCCTGAGTTCGCCATCTTCAGA | PEFAIFRKEGNFSDLKE |
| | | | AAAGAGGGCAACTTCTCCGACCTGAAAGAGAAAGTCAACGCCATCGA | KVNAIEREKAEKFRKL |
| | | | GAGAGAGAAGGCCGAGAAGTTCCGGAAGCTGCAGGACGCCTCCAGA | QDASRSAQALVEQMV |
| | | | TCTGCCCAGGCTCTGGTGGAACAGATGGTCAACGAAGGCGTGAACGC | NEGVNADSIKQASEQL |
| | | | CGACAGCATCAAGCAGGCCTCAGAGCAGCTGAACTCCCGGTGGATCG | NSRWIEFCQLLSERLN |
| | | | AGTTCTGTCAGCTCCTGAGCGAGAGACTGAACTGGCTGGAATACCAG | WLEYQNNIIAFYNQLQ |
| | | | AACAATATCATTGCCTTCTACAACCAGCTCCAGCAGCTCGAACAGAT | QLEQMTTTAENWLKIQ |
| | | | GACCACCACAGCCGAGAATTGGCTGAAGATCCAGCCTACCACACCTA | PTTPSEPTAIKSQLKICK |
| | | | GCGAGCCCACCGCCATTAAGAGCCAGCTGAAAATCTGCAAGGACGAA | DEVNRLSDLQPQIERL |
| | | | GTGAATCGGCTGAGCGATCTGCAGCCCCAGATCGAAAGACTGAAAAT | KIQSIALKEKGQGPMFL |
| | | | CCAGTCTATCGCCCTCAAAGAGAAAGGACAGGGCCCCATGTTCCTGG | DADFVAFTNHFKQVFS |
| | | | ACGCCGATTTTGTGGCCTTTACCAACCACTTCAAACAGGTGTTCTCCG | DVQAREKELQTIFDTLP |
| | | | ACGTGCAGGCCCGGGAAAAAGAGCTGCAGACCATCTTCGACACCCTG | PMRYQETMSAIRTWV |
| | | | CCTCCAATGAGATACCAAGAGACAATGAGCGCCATCCGGACCTGGGT | QQSETKLSIPQLSVTDY |
| | | | GCAGCAAAGCGAGACAAAGCTGAGCATCCCACAGCTGTCCGTGACCG | EIMEQRLGELQALQSS |
| | | | ACTACGAGATCATGGAACAGAGACTGGGCGAACTGCAGGCCCTCCAG | LQEQQSGLYYLSTTVK |
| | | | TCTAGCCTGCAAGAACAGCAGTCCGGCCTGTACTACCTGAGCACAAC | EMSKKAPSEISRKYQSE |
| | | | CGTGAAAGAGATGAGCAAGAAGGCCCCTAGCGAGATCTCCCGGAAG | FEEIEGRWKKLSSQLV |
| | | | TACCAGAGCGAGTTCGAAGAGATCGAAGGCCGGTGGAAGAAGCTGT | EHCQKLEEQMNKLRKI |
| | | | CTAGCCAGCTGGTTGAGCACTGCCAGAAACTCGAGGAACAAATGAAC | QNHIQTLKKWMAEVD |
| | | | AAGCTGCGCAAGATCCAGAATCACATCCAGACGCTGAAGAAATGGAT | VFLKEEWPALGDSEIL |
| | | | GGCCGAGGTGGACGTGTTCCTGAAAGAAGAGTGGCCCGCTCTGGGCG | KKQLKQCRLLVSDIQTI |
| | | | ACTCCGAGATTCTGAAGAAACAGCTCAAACAGTGCCGGCTGCTGGTG | QPSLNSVNEGGQKIKN |
| | | | TCCGATATCCAGACAATCCAGCCAAGCCTGAACTCCGTGAATGAAGG | EAEPEFASRLETELKEL |
| | | | CGGCCAGAAGATCAAGAACGAGGCCGAGCCTGAGTTTGCCAGCAGA | NTQWDHMCQQVYAR |
| | | | CTGGAAACCGAACTGAAAGAACTCAACACCCAGTGGGACCACATGTG | KEALKGGLEKTVSLQK |
| | | | CCAGCAAGTGTACGCCCGGAAAGAGGCCCTGAAAGGCGGACTGGAA | DLSEMHEWMTQAEEE |
| | | | AAAACAGTGTCTCTGCAGAAAGACCTCAGCGAGATGCACGAGTGGAT | YLERDFEYKTPDELQK |
| | | | GACCCAGGCCGAAGAGGAATACCTGGAACGGGACTTCGAGTACAAG | AVEEMKRAKEEAQQK |
| | | | ACCCCTGATGAGCTGCAAAAAGCCGTCGAGGAAATGAAGCGGGCCA | EAKVKLLTESVNSVIA |
| | | | AAGAAGAGGCCCAGCAGAAAGAAGCCAAAGTCAAGCTGCTGACCGA | QAPPVAQEALKKELET |
| | | | GTCCGTGAATAGCGTTATCGCTCAGGCCCCTCCTGTGGCTCAAGAAGC | LTTNYQWLCTRLNGK |
| | | | TCTGAAAAAAGAACTCGAGACACTGACCACCAACTACCAGTGGCTGT | CKTLEEVWACWHELL |
| | | | GCACCAGACTGAACGGCAAGTGCAAGACACTGGAAGAAGTGTGGGC | SYLEKANKWLNEVEF |
| | | | CTGCTGGCACGAACTGCTGTCCTATCTGGAAAAGGCCAACAAGTGGC | KLKTTENIPGGAEEISE |
| | | | TGAACGAGGTGGAATTCAAGCTGAAAACCACCGAGAACATCCCTGGC | VLDSLENLMRHSEDNP |
| | | | GGCGCTGAAGAGATCAGCGAAGTGCTGGACAGCCTGGAAAACCTGAT | NQIRILAQTLTDGGVM |
| | | | GAGACACAGCGAGGACAACCCCAATCAGATCAGAATCCTGGCTCAGA | DELINEELETFNSRWRE |
| | | | CCCTGACCGATGGCGGCGTGATGGACGAGCTGATCAACGAGGAACTG | LHEEAVRRQKLLEQSI |
| | | | GAAACATTCAACAGCCGGTGGCGGGAACTGCACGAGGAAGCTGTTCG | QSAQETEKSLHLIQESL |
| | | | GAGACAGAAACTCCTCGAGCAGTCTATCCAGAGCGCACAAGAAACCG | TFIDKQLAAYIADKVD |
| | | | AGAAGTCCCTGCACCTGATCCAAGAGAGCCTGACCTTCATCGACAAG | AAQMPQEAQKIQSDLT |
| | | | CAGCTGGCCGCTTATATCGCCGACAAGGTGGACGCTGCTCAGATGCC | SHEISLEEMKKHNQGK |
| | | | TCAAGAGGCTCAGAAGATTCAGAGCGACCTGACCAGCCACGAGATTA | EAAQRVLSQIDVAQKK |
| | | | GCCTGGAAGAAATGAAGAAGCACAACCAGGGCAAAGAGGCCGCTCA | LQDVSMKFRLFQKPAN |
| | | | GAGAGTCCTGAGCCAGATCGATGTGGCCCAGAAAAAACTCCAGGACG | FEQRLQESKMILDEVK |
| | | | TGTCCATGAAGTTTCGGCTGTTTCAGAAGCCCGCCAACTTCGAGCAGA | MHLPALETKSVEQEVV |
| | | | GACTGCAAGAGTCCAAGATGATCCTGGACGAAGTCAAAATGCATCTG | QSQLNHCVNLYKSLSE |
| | | | CCCGCTCTCGAGACTAAGAGCGTGGAACAAGAAGTGGTGCAGTCCCA | VKSEVEMVIKTGRQIV |
| | | | GCTGAACCACTGCGTGAACCTGTACAAGAGCCTGTCCGAAGTGAAGT | QKKQTENPKELDERVT |
| | | | CTGAGGTGGAAATGGTCATCAAGACCGGCAGGCAGATCGTCCAGAAG | ALKLHYNELGAKVTER |
| | | | AAGCAGACCGAGAATCCCAAAGAACTGGACGAGAGAGTGACCGCTC | KQQLEKCLKLSRKMR |
| | | | TGAAGCTGCACTACAATGAGCTGGGCGCCAAAGTGACCGAGCGGAA | KEMNVLTEWLAATDM |
| | | | GCAGCAACTCGAAAAGTGCCTGAAGCTGAGCCGCAAGATGCGGAAA | ELTKRSAVEGMPSNLD |
| | | | GAAATGAACGTGCTGACAGAGTGGCTGGCCGCCACCGATATGGAACT | SEVAWGKATQKEIEKQ |
| | | | GACTAAGAGAAGCGCCGTGGAAGGCATGCCCAGCAACCTGGATAGT | KVHLKSITEVGEALKT |
| | | | GAAGTGGCCTGGGGCAAAGCCACTCAGAAAGAGATTGAGAAGCAGA | VLGKKETLVEDKLSLL |
| | | | AGGTCCACCTGAAGTCCATCACCGAAGTGGGCGAAGCCCTGAAAACC | NSNWIAVTSRAEEWLN |
| | | | GTGCTGGGCAAGAAAGAAACCCTGGTCGAGGACAAGCTGTCCCTGCT | LLLEYQKHMETFDQN |
| | | | GAATTCCAACTGGATCGCCGTGACCTCCAGAGCTGAGGAATGGCTGA | VDHITKWIIQADTLLDE |
| | | | ATCTGCTGCTCGAGTACCAGAAACACATGGAAACTTTCGACCAGAAC | SEKKKPQQKEDVLKRL |
| | | | GTCGACCACATCACAAAGTGGATCATCCAGGCTGATACCCTGCTGGA | KAELNDIRPKVDSTRD |
| | | | CGAGTCCGAGAAGAAGAAACCCCAACAAAAAGAAGATGTGCTGAAG | QAANLMANRGDHCRK |
| | | | CGGCTGAAAGCCGAGCTGAATGACATCCGGCCTAAGGTGGACAGCAC | LVEPQISELNHRFAAIS |
| | | | CAGAGATCAGGCAGCCAACCTGATGGCCAACAGAGGCGACCACTGTC | HRIKTGKASIPLKELEQ |
| | | | GGAAGCTGGTCGAGCCTCAGATTAGCGAACTGAACCACAGATTTGCC | FNSDIQKLLEPLEAEIQ |
| | | | GCCATCAGCCACCGGATCAAGACAGGCAAGGCCAGCATTCCTCTCAA | QGVNLKEEDFNKDMN |
| | | | AGAGCTTGAGCAGTTCAACAGCGACATCCAAAAGCTGCTCGAACCCC | EDNEGTVKELLQRGDN |
| | | | TGGAAGCCGAGATCCAACAGGGCGTCAACCTCAAAGAAGAAGATTTC | LQQRITDERKREEIKIK |
| | | | AACAAGGACATGAACGAGGACAATGAGGGCACCGTCAAAGAATTGC | QQLLQTKHNALKDLRS |
| | | | TGCAGCGGGGCGACAATCTGCAGCAGCGGATTACCGATGAGCGCAAG | QRRKKALEISHQWYQ |
| | | | AGAGAGGAAATCAAGATCAAGCAGCAGCTCCTGCAGACAAAGCACA | YKRQADDLLKCLDDIE |
| | | | ACGCACTGAAGGACCTGAGAAGCCAGCGGCGGAAGAAGGCACTGGA | KKLASLPEPRDERKIKE |
| | | | AATCTCCCACCAGTGGTATCAGTACAAGCGGCAGGCCGACGACCTGC | IDRELQKKKEELNAVR |
| | | | TCAAGTGCCTGGACGACATCGAAAAAAAGCTGGCCTCTCTGCCCGAG | RQAEGLSEDGAAMAV |
| | | | CCTAGGGACGAGCGGAAAATCAAAGAAATCGACCGGGAACTCCAGA | EPTQIQLSKRWREIESK |
| | | | AAAAGAAAGAGGAACTGAACGCCGTCAGAAGGCAGGCTGAGGGCCT | FAQFRRLNFAQIHTVR |
| | | | TAGTGAAGATGGGGCCGCTATGGCCGTGGAACCCACACAGATCCAAC | EETMMVMTEDMPLEIS |
| | | | TGTCCAAGCGCTGGCGGGAAATCGAGAGCAAGTTTGCCCAGTTCCGG | YVPSTYLTEITHVSQAL |
| | | | CGGCTGAATTTCGCCCAGATTCACACCGTGCGCGAAGAAACCATGAT | LEVEQLLNAPDLCAKD |
| | | | GGTTATGACCGAGGACATGCCCCTGGAAATCAGCTACGTGCCCTCTA | FEDLFKQEESLKNIKDS |
| | | | CCTACCTGACCGAGATCACCCATGTGTCACAGGCCCTGCTTGAGGTCG | LQQSSGRIDIIHSKKTA |
| | | | AACAGCTGCTGAACGCCCCTGATCTGTGCGCCAAGGATTTCGAGGAT | ALQSATPVERVKLQEA |
| | | | CTGTTCAAGCAAGAGGAATCCCTCAAGAATATCAAGGACAGCCTCCA | LSQLDFQWEKVNKMY |
| | | | GCAAAGCAGCGGCCGGATCGATATCATCCACAGCAAGAAAACCGCC | KDRQGRFDRSVEKWR |
| | | | GCACTGCAGTCCGCCACACCTGTGGAAAGAGTGAAACTGCAAGAAGC | RFHYDIKIFNQWLTEA |
| | | | ACTGTCTCAGCTGGACTTCCAGTGGGAGAAAGTGAACAAGATGTACA | EQFLRKTQIPENWEHA |
| | | | AGGACAGGCAGGGCAGATTCGACCGCAGCGTGGAAAAGTGGCGGCG | KYKWYLKELQDGIGQ |
| | | | GTTCCACTACGACATCAAGATCTTCAATCAGTGGCTGACTGAGGCAG | RQTVVRTLNATGEEIIQ |
| | | | AGCAGTTCCTGAGAAAGACTCAGATCCCCGAGAACTGGGAGCACGCC | QSSKTDASILQEKLGSL |
| | | | AAGTACAAGTGGTATCTTAAAGAGCTTCAGGACGGCATCGGCCAGAG | NLRWQEVCKQLSDRK |
| | | | GCAGACAGTCGTCAGAACACTGAATGCCACCGGCGAGGAAATTATCC | KRLEEQKNILSEFQRDL |
| | | | AGCAGTCTAGCAAGACCGACGCCAGCATCCTGCAAGAGAAGCTGGG | NEFVLWLEEADNIASIP |
| | | | ATCCCTGAACCTGCGGTGGCAAGAAGTGTGCAAGCAGCTGAGCGACC | LEPGKEQQLKEKLEQV |
| | | | GGAAGAAGCGGCTCGAAGAACAGAAGAATATCCTGTCCGAGTTCCAG | KLLVEELPLRQGILKQL |
| | | | CGGGACCTGAACGAGTTTGTGCTGTGGCTCGAGGAAGCCGACAATAT | NETGGPVLVSAPISPEE |
| | | | CGCTAGCATCCCTCTGGAACCCGGCAAAGAGCAACAGCTTAAAGAAA | QDKLENKLKQTNLQWI |
| | | | AACTGGAACAAGTGAAGCTCCTGGTTGAGGAACTGCCCCTGAGACAG | KVSRALPEKQGEIEAQI |
| | | | GGCATCCTGAAGCAACTGAACGAGACAGGCGGCCCTGTGCTGGTTTC | KDLGQLEKKLEDLEEQ |
| | | | TGCCCCTATCTCACCCGAGGAACAGGATAAGCTCGAGAACAAACTCA | LNHLLLWLSPIRNQLEI |
| | | | AGCAGACAAACCTCCAGTGGATCAAGGTGTCCCGGGCACTGCCTGAG | YNQPNQEGPFDVKETE |
| | | | AAACAGGGCGAAATCGAGGCCCAGATCAAGGACCTGGGACAGCTCG | IAVQAKQPDVEEILSKG |
| | | | AAAAGAAGCTCGAAGATTTGGAGGAACAACTCAACCACCTCCTGCTC | QHLYKEKPATQPVKRK |
| | | | TGGCTGAGCCCTATCCGGAATCAGCTCGAGATCTATAACCAGCCTAAT | LEDLSSEWKAVNRLLQ |
| | | | CAAGAGGGCCCCTTCGACGTGAAAGAAACAGAGATCGCTGTGCAGGC | ELRAKQPDLAPGLTTIG |
| | | | CAAGCAGCCAGATGTGGAAGAGATCCTGTCCAAGGGACAGCATCTGT | ASPTQTVTLVTQPVVT |
| | | | ACAAAGAAAAGCCTGCCACACAGCCCGTGAAGCGCAAGTTGGAGGA | KETAISKLEMPSSLMLE |
| | | | CCTGTCTAGCGAGTGGAAGGCCGTGAACCGGCTGCTCCAAGAACTGA | VPALADFNRAWTELTD |
| | | | GAGCCAAACAGCCCGATCTGGCCCCTGGCCTGACAACAATTGGAGCT | WLSLLDQVIKSQRVMV |
| | | | AGCCCCACTCAGACCGTGACACTGGTTACACAGCCAGTGGTCACCAA | GDLEDINEMIIKQKAT |
| | | | AGAGACAGCCATCTCCAAACTGGAAATGCCCTCTAGCCTGATGTTGG | MQDLEQRRPQLEELIT |
| | | | AGGTGCCCGCACTGGCCGACTTCAATAGAGCTTGGACCGAGCTGACT | AAQNLKNKTSNQEAR |
| | | | GATTGGCTGTCCCTCCTGGACCAAGTGATCAAGAGCCAGAGAGTGAT | TIITDRIERIQNQWDEV |
| | | | GGTCGGAGATCTTGAGGACATCAACGAAATGATTATCAAGCAGAAAG | QEHLQNRRQQLNEML |
| | | | CCACCATGCAGGATCTCGAACAGCGCAGACCCCAGCTTGAAGAACTG | KDSTQWLEAKEEAEQ |
| | | | ATCACAGCCGCACAGAATCTGAAAAACAAGACCAGCAATCAAGAAG | VLGQARAKLESWKEG |
| | | | CCAGGACCATCATCACCGACCGGATCGAGCGGATTCAGAACCAGTGG | PYTVDAIQKKITETKQL |
| | | | GATGAAGTCCAAGAGCATCTGCAGAACCGGCGGCAGCAGCTTAACGA | AKDLRQWQTNVDVAN |
| | | | AATGCTGAAGGACAGCACACAGTGGCTCGAAGCTAAAGAAGAAGCC | DLALKLLRDYSADDTR |
| | | | GAGCAGGTTCTGGGACAAGCCAGAGCCAAGCTGGAATCCTGGAAAG | KVHMITENINASWRSI |
| | | | AGGGACCCTACACCGTCGACGCCATTCAGAAAAAAATCACCGAGACA | HKRVSEREAALEETHR |
| | | | AAACAGCTGGCCAAGGATCTGCGGCAGTGGCAGACCAATGTGGACGT | LLQQFPLDLEKFLAWL |
| | | | GGCAAATGACCTGGCTCTGAAACTGCTCCGGGACTACAGCGCCGACG | TEAETTANVLQDATRK |
| | | | ACACCAGAAAGGTGCACATGATCACAGAGAACATCAACGCCTCTTGG | ERLLEDSKGVKELMKQ |
| | | | CGGAGCATCCACAAGCGGGTGTCCGAAAGAGAGGCTGCCCTCGAGG | WQDLQGEIEAHTDVY |
| | | | AAACACACAGACTGCTGCAACAGTTTCCCCTGGACCTTGAGAAATTC | HNLDENSQKILRSLEGS |
| | | | CTGGCCTGGCTCACCGAGGCCGAAACCACAGCTAATGTCCTCCAGGA | DDAVLLQRRLDNMNF |
| | | | TGCCACACGGAAAGAGCGGTTGCTGGAAGATAGCAAAGGGGTCAAA | KWSELRKKSLNIRSHL |
| | | | GAACTTATGAAGCAATGGCAGGACCTCCAAGGGGAGATCGAGGCTCA | EASSDQWKRLHLSLQE |
| | | | CACCGACGTGTACCACAATCTGGATGAGAACAGTCAGAAGATCCTGC | LLVWLQLKDDELSRQ |
| | | | GGTCCCTGGAAGGCTCCGATGATGCTGTGTTGCTCCAGCGGAGACTG | APIGGDFPAVQKQNDV |
| | | | GATAACATGAACTTCAAGTGGAGCGAGCTGCGGAAAAAGTCCCTGAA | HRAFKRELKTKEPVIM |
| | | | CATCAGAAGCCATCTGGAAGCCAGCAGCGACCAGTGGAAAAGACTCC | STLETVRIFLTEQPLEG |
| | | | ACCTGAGCCTCCAAGAGCTTCTCGTGTGGCTGCAGCTCAAGGACGAT | LEKLYQEPRELPPEERA |
| | | | GAGCTGAGCAGACAGGCCCCAATCGGCGGAGATTTTCCCGCCGTGCA | QNVTRLLRKQAEEVNT |
| | | | GAAACAGAACGACGTGCACAGAGCCTTCAAGCGGGAACTCAAGACC | EWEKLNLHSADWQRK |
| | | | AAAGAACCCGTGATTATGAGCACCCTGGAAACTGTGCGGATCTTTCT | IDETLERLRELQEATDE |
| | | | GACCGAGCAGCCTCTGGAAGGACTCGAGAAGCTGTACCAAGAACCTA | LDLKLRQAEVIKGSWQ |
| | | | GAGAGCTGCCTCCTGAGGAACGGGCCCAGAACGTTACACGGCTGCTG | PVGDLLIDSLQDHLEK |
| | | | AGAAAACAGGCCGAGGAAGTGAATACCGAGTGGGAAAAGCTGAATC | VKALRGEIAPLKENVS |
| | | | TCCACTCCGCCGACTGGCAGCGGAAGATCGATGAGACTCTGGAAAGG | HVNDLARQLTTLGIQL |
| | | | CTTCAAGAATTGCAAGAGGCCACCGACGAGCTGGACCTGAAGCTTAG | SPYNLSTLEDLNTRWK |
| | | | ACAGGCTGAAGTCATCAAAGGCAGCTGGCAGCCTGTGGGCGACCTCC | LLQVAVEDRVRQLHE |
| | | | TGATTGATAGCCTGCAGGACCACCTCGAAAAAGTGAAGGCCCTGCGG | AHRDFGPASQHFLSTS |
| | | | GGAGAGATCGCCCCTCTGAAAGAAAACGTGTCCCACGTGAACGACCT | VQGPWERAISPNKVPY |
| | | | GGCACGGCAGCTTACAACACTGGGGATTCAGCTGAGCCCCTACAACC | YINHETQTTCWDHPK |
| | | | TGTCCACACTCGAGGACCTGAATACCCGATGGAAGCTCCTCCAGGTG | MTELYQSLADLNNVRF |
| | | | GCCGTTGAAGATAGAGTGCGGCAGCTGCACGAAGCCCACAGAGATTT | SAYRTAMKLRRLQKA |
| | | | TGGACCAGCCAGCCAGCACTTTCTGAGCACCTCAGTGCAAGGCCCCT | LCLDLLSLSAACDALD |
| | | | GGGAGAGAGCTATCAGCCCTAACAAGGTGCCCTACTACATCAACCAC | QHNLKQNDQPMDILQI |
| | | | GAGACACAGACAACCTGTTGGGATCACCCCAAGATGACCGAACTGTA | INCLTTIYDRLEQEHNN |
| | | | TCAGTCCCTGGCCGATCTGAACAATGTGCGGTTCAGCGCCTACCGGAC | LVNVPLCVDMCLNWL |
| | | | CGCAATGAAGCTGCGGAGACTTCAGAAAGCCCTGTGCCTCGACCTGC | LNVYDTGRTGRIRVLS |
| | | | TGTCTCTGAGTGCAGCATGTGATGCCCTGGACCAGCATAATCTGAAGC | FKTGIISLCKAHLEDKY |
| | | | AGAACGACCAGCCTATGGACATCCTCCAGATCATCAACTGCCTGACC | RYLFKQVASSTGFCDQ |
| | | | ACTATCTACGACCGGCTCGAACAAGAACACAACAACCTCGTGAACGT | RRLGLLLHDSIQIPRQL |
| | | | GCCCCTGTGCGTGGACATGTGCCTCAATTGGCTGCTGAATGTGTACGA | GEVASFGGSNIEPSVRS |
| | | | CACCGGCCGGACCGGCAGAATCAGAGTGCTGTCTTTCAAAACCGGCA | CFQFANNKPEIEAALFL |
| | | | TCATCTCCCTGTGCAAAGCCCATCTCGAGGATAAGTACCGCTACCTGT | DWMRLEPQSMVWLPV |
| | | | TTAAGCAGGTCGCCAGCAGCACCGGCTTTTGCGATCAAAGAAGGCTG | LHRVAAAETAKHQAK |
| | | | GGACTCCTGCTGCACGACAGCATTCAGATCCCAAGACAGCTGGGCGA | CNICKECPIIGFRYRSLK |
| | | | AGTGGCTAGCTTCGGCGGCTCTAATATCGAGCCTAGCGTGCGGAGCT | HFNYDICQSCFFSGRV |
| | | | GCTTCCAGTTCGCCAACAACAAGCCCGAGATTGAGGCAGCCCTCTTCC | AKGHKMHYPMVEYCT |
| | | | TGGATTGGATGCGGCTGGAACCCCAGAGCATGGTTTGGCTGCCAGTG | PTTSGEDVRDFAKVLK |
| | | | CTGCATAGAGTGGCCGCTGCCGAAACAGCTAAGCACCAGGCCAAGTG | NKFRTKRYFAKHPRM |
| | | | CAACATCTGCAAAGAGTGCCCCATCATCGGCTTCCGGTACAGAAGCC | GYLPVQTVLEGDNME |
| | | | TGAAGCACTTCAACTACGATATCTGCCAGTCTTGTTTCTTCTCTGGCCG | TPVTLINFWPVDSAPAS |
| | | | CGTGGCCAAGGGCCACAAGATGCATTACCCCATGGTGGAATACTGCA | SPQLSHDDTHSRIEHYA |
| | | | CCCCTACCACCTCCGGCGAAGATGTCCGGGATTTTGCCAAGGTGCTCA | SRLAEMENSNGSYLND |
| | | | AAAACAAGTTCCGCACCAAGCGCTACTTCGCAAAGCACCCCAGAATG | SISPNESIDDEHLLIQHY |
| | | | GGCTACCTGCCTGTGCAGACAGTTCTGGAAGGCGATAATATGGAAAC | CQSLNQDSPLSQPRSPA |
| | | | CCCTGTGACGCTGATCAATTTCTGGCCCGTGGATAGCGCCCCTGCCAG | QILISLESEERGELERIL |
| | | | TTCTCCTCAGCTGTCTCACGATGACACCCACTCCAGAATCGAGCACTA | ADLEEENRNLQAEYDR |
| | | | CGCCTCCAGACTGGCCGAGATGGAAAACAGCAACGGCAGCTATCTGA | LKQQHEHKGLSPLPSPP |
| | | | ACGATAGCATCAGCCCCAACGAGAGCATCGACGACGAACATCTGCTC | EMMPTSPQSPRDAELI |
| | | | ATCCAGCACTACTGTCAGAGCCTGAACCAGGACTCCCCACTGAGCCA | AEAKLLRQHKGRLEAR |
| | | | GCCTAGAAGCCCAGCACAGATTCTGATTTCCCTGGAATCCGAAGAAC | MQILEDHNKQLESQLH |
| | | | GGGGCGAGCTGGAAAGAATTCTGGCCGACCTCGAGGAAGAAAACCG | RLRQLLEQPQAEAKVN |
| | | | GAACCTGCAGGCAGAGTACGACAGACTGAAACAACAGCACGAGCAC | GTTVSSPSTSLQRSDSS |
| | | | AAGGGACTGAGCCCTTTGCCATCTCCTCCAGAAATGATGCCCACCTCT | QPMLLRVVGSQTSDSM |
| | | | CCACAGAGCCCCAGAGATGCAGAGCTGATTGCCGAGGCCAAACTCCT | GEEDLLSPPQDTSTGLE |
| | | | GCGCCAGCACAAAGGCAGACTCGAGGCCAGAATGCAGATTCTCGAA | EVMEQLNNSFPSSRGR |
| | | | GATCACAACAAACAGCTCGAGTCTCAGCTCCACCGCCTGCGGCAATT | NTPGKPMREDTM (SEQ |
| | | | GCTGGAACAACCACAGGCAGAGGCTAAAGTGAACGGCACCACAGTG | ID NO: 4) |
| | | | TCTAGCCCCAGCACATCCCTGCAGAGATCCGATAGCTCCCAGCCAAT | |
| | | | GCTCCTGAGAGTCGTGGGCAGCCAGACCAGCGATTCTATGGGCGAAG | |
| | | | AAGATCTGCTTAGCCCTCCTCAGGATACAAGCACCGGCCTCGAAGAA | |
| | | | GTTATGGAACAACTTAACAATAGCTTCCCCAGCAGCAGAGGCCGCAA | |
| | | | CACACCTGGCAAGCCCATGCGGGAAGATACCATG (SEQ ID NO: 3) | |
| Full | Wild Type | NP_003997 | ATGCTGTGGTGGGAGGAAGTTGAGGATTGCTATGAGCGGGAAGATGT | MLWWEEVEDCYERED |
| Length | protein | | GCAGAAGAAGACGTTTACGAAATGGGTCAATGCTCAATTCTCGAAAT | VQKKTFTKWVNAQFS |
| human | (CO | | TTGGCAAACAACACATAGAGAACCTTTTCTCCGATCTGCAAGATGGT | KFGKQHIENLFSDLQD |
| dystrophin | nucleotide | | AGGAGATTATTGGATCTCCTCGAGGGGTTAACTGGGCAGAAACTGCC | GRRLLDLLEGLTGQKL |
| | seq-1A) | | CAAAGAGAAAGGTTCTACCAGAGTTCACGCGCTGAATAATGTAAATA | PKEKGSTRVHALNNVN |
| | | | AAGCCCTGAGAGTTCTTCAGAACAACAATGTGGACCTTGTCAATATTG | KALRVLQNNNVDLVNI |
| | | | GGTCTACAGATATAGTTGACGGGAATCACAAACTCACTCTTGGGCTC | GSTDIVDGNHKLTLGLI |
| | | | ATATGGAACATCATACTCCACTGGCAAGTTAAGAATGTAATGAAGAA | WNIILHWQVKNVMKN |
| | | | TATTATGGCGGGCCTGCAACAAACGAATTCCGAGAAAATCTTGCTGT | IMAGLQQTNSEKILLS |
| | | | CTTGGGTAAGACAGTCTACTCGGAATTATCCCCAGGTGAACGTGATA | WVRQSTRNYPQVNVIN |
| | | | AATTTTACCACTTCCTGGTCTGATGGGTTAGCGTTAAATGCCCTTATTC | FTTSWSDGLALNALIH |
| | | | ACTCTCACCGGCCCGACCTTTTCGATTGGAATTCCGTAGTTTGTCAAC | SHRPDLFDWNSVVCQ |
| | | | AATCCGCGACTCAACGGCTTGAACACGCCTTTAATATCGCCAGGTATC | QSATQRLEHAFNIARY |
| | | | AGCTCGGGATAGAGAAACTTTTAGATCCCGAGGATGTTGATACTACC | QLGIEKLLDPEDVDTT |
| | | | TATCCTGATAAGAAGTCAATACTCATGTATATAACCAGTTTATTTCAA | YPDKKSILMYITSLFQV |
| | | | GTTCTCCCACAGCAAGTTTCAATTGAGGCCATACAAGAGGTTGAGAT | LPQQVSIEAIQEVEMLP |
| | | | GCTGCCCAGACCACCTAAGGTTACAAAAGAAGAGCACTTTCAATTAC | RPPKVTKEEHFQLHHQ |
| | | | ATCACCAGATGCACTATAGCCAGCAAATTACCGTATCGTTAGCCCAG | MHYSQQITVSLAQGYE |
| | | | GGGTATGAGAGAACCTCTTCTCCCAAACCCCGGTTTAAGTCGTATGCC | RTSSPKPRFKSYAYTQ |
| | | | TATACACAAGCCGCCTATGTTACTACCTCAGACCCCACAAGATCTCCG | AAYVTTSDPTRSPFPSQ |
| | | | TTTCCCAGCCAACACTTAGAGGCTCCCGAGGATAAATCCTTTGGGTCT | HLEAPEDKSFGSSLME |
| | | | TCTCTGATGGAGTCTGAGGTGAATTTAGACCGGTATCAAACCGCCTTG | SEVNLDRYQTALEEVL |
| | | | GAGGAAGTACTCTCTTGGCTTCTGTCTGCCGAGGATACTCTGCAAGCC | SWLLSAEDTLQAQGEI |
| | | | CAAGGGGAGATATCTAATGATGTCGAGGTGGTTAAAGACCAGTTTCA | SNDVEVVKDQFHTHE |
| | | | CACCCACGAGGGGTATATGATGGATCTCACTGCCCACCAAGGCCGTG | GYMMDLTAHQGRVG |
| | | | TTGGGAATATACTACAACTTGGGTCTAAACTAATTGGGACCGGGAAA | NILQLGSKLIGTGKLSE |
| | | | CTTTCTGAGGATGAGGAGACCGAGGTACAAGAGCAGATGAATCTTCT | DEETEVQEQMNLLNSR |
| | | | GAATTCGAGGTGGGAGTGTTTACGAGTTGCCAGCATGGAGAAACAGT | WECLRVASMEKQSNL |
| | | | CCAATCTACATCGCGTTCTGATGGACTTACAAAATCAGAAGCTGAAA | HRVLMDLQNQKLKEL |
| | | | GAATTGAACGACTGGCTTACCAAAACTGAGGAGCGGACTCGTAAAAT | NDWLTKTEERTRKME |
| | | | GGAAGAGGAGCCTCTCGGGCCAGACCTAGAGGATCTGAAACGGCAG | EEPLGPDLEDLKRQVQ |
| | | | GTACAACAACATAAAGTGCTTCAGGAAGATCTTGAGCAGGAACAAGT | QHKVLQEDLEQEQVR |
| | | | TAGGGTAAACTCGCTAACCCACATGGTTGTCGTTGTTGATGAGTCCTC | VNSLTHMVVVVDESS |
| | | | TGGGGATCACGCCACAGCCGCCTTGGAAGAACAACTCAAGGTATTAG | GDHATAALEEQLKVL |
| | | | GGGATCGGTGGGCCAATATTTGCCGGTGGACTGAGGACCGGTGGGTC | GDRWANICRWTEDRW |
| | | | CTCCTACAGGATATTCTGCTAAAATGGCAACGGCTTACCGAAGAGCA | VLLQDILLKWQRLTEE |
| | | | ATGTCTCTTTAGTGCCTGGTTATCCGAGAAAGAGGATGCCGTAAACA | QCLFSAWLSEKEDAVN |
| | | | AGATCCACACTACTGGGTTCAAAGACCAAAATGAGATGCTCAGCTCT | KIHTTGFKDQNEMLSS |
| | | | CTCCAGAAACTGGCCGTCCTTAAAGCCGACTTGGAGAAGAAGAAACA | LQKLAVLKADLEKKK |
| | | | GTCTATGGGGAAACTCTATTCATTGAAACAGGATCTGCTTAGCACCCT | QSMGKLYSLKQDLLST |
| | | | CAAGAATAAGTCTGTCACACAGAAAACCGAGGCCTGGTTAGACAATT | LKNKSVTQKTEAWLD |
| | | | TTGCCAGGTGCTGGGATAATCTTGTCCAAAAGCTCGAGAAATCTACTG | NFARCWDNLVQKLEK |
| | | | CCCAGATTTCTCAAGCTGTTACTACAACTCAACCCTCTCTGACTCAAA | STAQISQAVTTTQPSLT |
| | | | CCACTGTTATGGAGACTGTGACAACTGTAACTACTAGAGAGCAGATA | QTTVMETVTTVTTREQ |
| | | | CTGGTTAAACACGCGCAAGAAGAGTTACCTCCTCCTCCGCCCCAAAA | ILVKHAQEELPPPPPQK |
| | | | GAAAAGGCAGATCACTGTGGATTCGGAAATACGCAAAAGACTCGATG | KRQITVDSEIRKRLDVD |
| | | | TTGATATAACTGAGCTGCATTCTTGGATTACACGGTCTGAGGCCGTAC | ITELHSWITRSEAVLQS |
| | | | TCCAATCTCCAGAATTTGCGATCTTTAGGAAAGAGGGCAATTTCTCAG | PEFAIFRKEGNFSDLKE |
| | | | ACCTTAAGGAGAAGGTAAATGCTATAGAGCGGGAGAAAGCTGAGAA | KVNAIEREKAEKFRKL |
| | | | GTTTCGCAAACTACAAGATGCCTCTCGGTCTGCCCAAGCCCTGGTTGA | QDASRSAQALVEQMV |
| | | | GCAAATGGTTAATGAAGGGGTAAATGCCGATTCCATTAAACAGGCCT | NEGVNADSIKQASEQL |
| | | | CTGAACAACTGAACTCCCGGTGGATTGAATTCTGTCAACTGCTTAGTG | NSRWIEFCQLLSERLN |
| | | | AGAGACTTAATTGGCTCGAGTATCAAAACAATATAATAGCCTTCTAC | WLEYQNNIIAFYNQLQ |
| | | | AATCAGTTACAGCAACTTGAGCAGATGACTACTACTGCCGAAAACTG | QLEQMTTTAENWLKIQ |
| | | | GCTGAAAATACAACCCACTACTCCCTCTGAGCCCACTGCCATTAAATC | PTTPSEPTAIKSQLKICK |
| | | | GCAGCTCAAAATCTGCAAAGACGAGGTTAATAGACTCTCCGATCTTC | DEVNRLSDLQPQIERL |
| | | | AGCCCCAAATAGAACGGCTTAAAATTCAGAGTATCGCCTTGAAAGAA | KIQSIALKEKGQGPMFL |
| | | | AAGGGGCAAGGGCCCATGTTTCTCGATGCCGATTTCGTCGCGTTTACC | DADFVAFTNHFKQVFS |
| | | | AACCATTTCAAACAGGTATTTTCCGACGTTCAAGCCCGGGAGAAAGA | DVQAREKELQTIFDTLP |
| | | | GTTACAGACCATTTTCGACACTTTACCGCCCATGCGGTATCAAGAGAC | PMRYQETMSAIRTWV |
| | | | CATGTCTGCCATTAGAACCTGGGTCCAACAATCGGAAACTAAGCTTTC | QQSETKLSIPQLSVTDY |
| | | | CATCCCTCAATTATCTGTGACTGACTATGAGATAATGGAGCAACGGCT | EIMEQRLGELQALQSS |
| | | | TGGGGAGCTGCAGGCCCTTCAAAGCTCTTTACAAGAGCAACAATCTG | LQEQQSGLYYLSTTVK |
| | | | GGCTCTATTATCTCTCCACTACGGTGAAGGAAATGTCCAAGAAAGCG | EMSKKAPSEISRKYQSE |
| | | | CCCTCCGAGATTTCTCGGAAATACCAAAGTGAGTTCGAGGAGATAGA | FEEIEGRWKKLSSQLV |
| | | | GGGGCGGTGGAAGAAACTGAGTTCTCAGCTTGTAGAGCATTGTCAAA | EHCQKLEEQMNKLRKI |
| | | | AGCTGGAAGAGCAAATGAACAAGCTCAGGAAAATCCAGAACCATAT | QNHIQTLKKWMAEVD |
| | | | CCAAACTCTCAAGAAATGGATGGCCGAGGTTGACGTCTTCTTGAAAG | VFLKEEWPALGDSEIL |
| | | | AGGAATGGCCGGCTCTTGGGGATAGCGAGATTCTAAAGAAGCAGTTG | KKQLKQCRLLVSDIQTI |
| | | | AAACAGTGCCGGCTCTTAGTTAGTGATATCCAGACCATACAACCCTCC | QPSLNSVNEGGQKIKN |
| | | | CTGAATTCTGTTAATGAGGGCGGTCAAAAGATTAAGAACGAGGCCGA | EAEPEFASRLETELKEL |
| | | | GCCCGAGTTTGCTTCAAGACTCGAGACTGAATTGAAAGAGCTGAATA | NTQWDHMCQQVYAR |
| | | | CACAGTGGGACCACATGTGTCAGCAAGTTTATGCCCGGAAAGAAGCG | KEALKGGLEKTVSLQK |
| | | | CTCAAAGGTGGGCTTGAGAAGACTGTTAGCTTACAGAAAGACCTTAG | DLSEMHEWMTQAEEE |
| | | | TGAGATGCACGAGTGGATGACCCAAGCTGAGGAGGAGTATCTTGAGC | YLERDFEYKTPDELQK |
| | | | GTGACTTTGAGTACAAAACACCCGACGAGTTACAGAAAGCTGTTGAA | AVEEMKRAKEEAQQK |
| | | | GAAATGAAGCGGGCGAAAGAAGAGGCACAACAGAAAGAGGCCAAA | EAKVKLLTESVNSVIA |
| | | | GTAAAGCTTCTGACAGAGTCTGTTAATTCGGTGATAGCCCAAGCGCCT | QAPPVAQEALKKELET |
| | | | CCCGTTGCGCAAGAGGCCTTGAAGAAAGAACTCGAGACCCTAACCAC | LTTNYQWLCTRLNGK |
| | | | AAATTATCAGTGGCTCTGCACTAGATTGAATGGGAAATGCAAAACGC | CKTLEEVWACWHELL |
| | | | TTGAGGAGGTTTGGGCTTGCTGGCACGAGTTGCTCTCCTATTTAGAAA | SYLEKANKWLNEVEF |
| | | | AGGCCAACAAATGGCTTAATGAAGTGGAATTTAAGCTCAAAACCACA | KLKTTENIPGGAEEISE |
| | | | GAGAATATACCCGGTGGAGCTGAGGAGATTTCTGAAGTTCTTGACTCT | VLDSLENLMRHSEDNP |
| | | | CTCGAGAATCTCATGCGGCATAGTGAAGACAATCCCAACCAAATACG | NQIRILAQTLTDGGVM |
| | | | GATTCTAGCCCAAACTCTGACTGACGGAGGGGTCATGGATGAGCTAA | DELINEELETFNSRWRE |
| | | | TTAACGAGGAGCTCGAGACTTTTAATTCCAGGTGGCGGGAGTTGCAC | LHEEAVRRQKLLEQSI |
| | | | GAGGAGGCCGTTCGGAGACAGAAATTGTTAGAGCAGTCCATTCAATC | QSAQETEKSLHLIQESL |
| | | | CGCCCAAGAGACCGAGAAATCGCTGCATCTCATACAGGAAAGCTTGA | TFIDKQLAAYIADKVD |
| | | | CCTTTATTGATAAGCAGCTCGCCGCCTATATAGCCGATAAAGTTGACG | AAQMPQEAQKIQSDLT |
| | | | CTGCCCAAATGCCCCAAGAAGCCCAGAAAATACAGTCTGACCTGACT | SHEISLEEMKKHNQGK |
| | | | AGCCATGAGATTTCTTTAGAGGAGATGAAGAAACACAACCAGGGTAA | EAAQRVLSQIDVAQKK |
| | | | AGAGGCGGCACAGCGGGTTCTTTCACAAATAGACGTTGCCCAGAAGA | LQDVSMKFRLFQKPAN |
| | | | AGCTCCAAGACGTCTCTATGAAATTTCGGCTCTTTCAGAAACCTGCCA | FEQRLQESKMILDEVK |
| | | | ATTTTGAGCAACGGTTACAAGAGTCCAAAATGATTCTCGATGAGGTC | MHLPALETKSVEQEVV |
| | | | AAAATGCACCTTCCGGCCCTGGAAACTAAAAGTGTTGAGCAAGAGGT | QSQLNHCVNLYKSLSE |
| | | | TGTACAGTCCCAATTAAATCACTGCGTCAACCTATATAAATCCCTATC | VKSEVEMVIKTGRQIV |
| | | | GGAGGTTAAATCTGAGGTCGAGATGGTAATTAAGACTGGGCGGCAAA | QKKQTENPKELDERVT |
| | | | TAGTCCAGAAGAAACAAACTGAGAATCCCAAAGAACTTGACGAGAG | ALKLHYNELGAKVTER |
| | | | AGTTACTGCCTTGAAACTGCACTACAATGAGCTTGGGGCGAAAGTTA | KQQLEKCLKLSRKMR |
| | | | CAGAGAGGAAACAGCAACTGGAAAAGTGCCTGAAACTCTCTCGGAA | KEMNVLTEWLAATDM |
| | | | AATGCGGAAAGAAATGAATGTGCTGACCGAGTGGCTAGCCGCCACTG | ELTKRSAVEGMPSNLD |
| | | | ACATGGAGTTAACTAAACGGTCTGCCGTAGAGGGGATGCCCTCTAAT | SEVAWGKATQKEIEKQ |
| | | | CTTGATTCAGAGGTTGCCTGGGGAAAAGCCACGCAAAAGGAGATCGA | KVHLKSITEVGEALKT |
| | | | GAAGCAGAAGGTCCATCTTAAGTCCATTACAGAGGTTGGGGAGGCCC | VLGKKETLVEDKLSLL |
| | | | TGAAAACCGTTTTAGGTAAGAAAGAAACCTTAGTTGAGGATAAACTC | NSNWIAVTSRAEEWLN |
| | | | TCACTGCTTAACTCAAATTGGATAGCCGTAACTAGCAGGGCCGAAGA | LLLEYQKHMETFDQN |
| | | | GTGGCTAAATTTGCTTCTGGAATACCAAAAGCATATGGAGACATTTG | VDHITKWIIQADTLLDE |
| | | | ATCAGAATGTCGACCACATCACGAAATGGATCATTCAGGCTGACACT | SEKKKPQQKEDVLKRL |
| | | | TTACTCGATGAATCCGAGAAGAAGAAGCCACAGCAAAAGGAGGACG | KAELNDIRPKVDSTRD |
| | | | TACTGAAACGACTCAAAGCCGAACTCAATGATATTAGGCCCAAAGTT | QAANLMANRGDHCRK |
| | | | GATTCTACTCGCGATCAAGCCGCCAATCTTATGGCCAATCGGGGCGAT | LVEPQISELNHRFAAIS |
| | | | CACTGCAGGAAATTGGTAGAGCCCCAGATTAGTGAGCTCAATCACAG | HRIKTGKASIPLKELEQ |
| | | | ATTTGCCGCCATATCCCACAGAATAAAGACAGGCAAAGCCTCTATTC | FNSDIQKLLEPLEAEIQ |
| | | | CCCTTAAGGAGCTCGAACAATTTAACTCGGACATTCAGAAACTCTTGG | QGVNLKEEDFNKDMN |
| | | | AGCCCTTAGAGGCCGAAATTCAACAAGGGGTTAATCTCAAAGAAGAG | EDNEGTVKELLQRGDN |
| | | | GACTTCAATAAGGACATGAATGAGGACAATGAGGGGACTGTTAAGG | LQQRITDERKREEIKIK |
| | | | AGCTTCTGCAAAGGGGCGACAATCTTCAACAACGGATTACTGATGAA | QQLLQTKHNALKDLRS |
| | | | CGGAAACGGGAGGAGATTAAGATTAAGCAGCAGCTTCTCCAAACAA | QRRKKALEISHQWYQ |
| | | | AACACAATGCGCTCAAAGACCTCAGATCTCAACGGCGGAAGAAAGCT | YKRQADDLLKCLDDIE |
| | | | TTGGAGATTTCTCACCAGTGGTACCAATACAAAAGACAGGCCGACGA | KKLASLPEPRDERKIKE |
| | | | TTTACTAAAATGCCTCGACGATATCGAGAAGAAGCTTGCTTCATTACC | IDRELQKKKEELNAVR |
| | | | CGAACCTCGGGACGAGCGGAAAATTAAAGAGATAGATCGCGAGCTA | RQAEGLSEDGAAMAV |
| | | | CAGAAGAAGAAAGAGGAGCTGAATGCCGTTAGACGGCAAGCCGAAG | EPTQIQLSKRWREIESK |
| | | | GGCTATCTGAAGACGGTGCAGCGATGGCGGTTGAGCCCACCCAAATT | FAQFRRLNFAQIHTVR |
| | | | CAGCTGTCCAAACGGTGGAGAGAGATAGAGTCTAAGTTTGCCCAATT | EETMMVMTEDMPLEIS |
| | | | CCGAAGACTCAATTTTGCCCAGATTCATACTGTTAGAGAGGAGACAA | YVPSTYLTEITHVSQAL |
| | | | TGATGGTGATGACTGAGGATATGCCCTTAGAGATATCGTATGTCCCCT | LEVEQLLNAPDLCAKD |
| | | | CTACCTATCTGACCGAGATTACTCATGTGTCTCAAGCCCTCCTCGAAG | FEDLFKQEESLKNIKDS |
| | | | TTGAGCAACTCTTAAATGCACCGGATCTTTGCGCCAAAGACTTCGAAG | LQQSSGRIDIIHSKKTA |
| | | | ATCTTTTCAAGCAGGAGGAGTCCCTTAAGAACATCAAAGACTCTCTCC | ALQSATPVERVKLQEA |
| | | | AGCAATCTAGTGGGCGCATAGATATCATTCACTCGAAGAAAACTGCA | LSQLDFQWEKVNKMY |
| | | | GCCCTCCAAAGCGCCACACCCGTTGAACGAGTTAAACTCCAAGAGGC | KDRQGRFDRSVEKWR |
| | | | CCTGTCACAGCTCGATTTTCAATGGGAGAAAGTCAACAAGATGTATA | RFHYDIKIFNQWLTEA |
| | | | AAGACCGGCAAGGGCGGTTTGACAGATCTGTTGAGAAATGGCGGCGG | EQFLRKTQIPENWEHA |
| | | | TTCCACTATGACATCAAAATATTTAATCAATGGCTAACTGAAGCCGAA | KYKWYLKELQDGIGQ |
| | | | CAGTTCTTACGGAAAACTCAGATTCCCGAGAATTGGGAGCACGCCAA | RQTVVRTLNATGEEIIQ |
| | | | ATACAAGTGGTACTTGAAAGAGCTACAAGACGGGATTGGCCAGCGGC | QSSKTDASILQEKLGSL |
| | | | AGACTGTTGTTCGGACTCTTAATGCCACCGGTGAGGAGATTATACAGC | NLRWQEVCKQLSDRK |
| | | | AAAGCTCTAAAACTGACGCATCTATCCTTCAAGAGAAACTCGGCTCTC | KRLEEQKNILSEFQRDL |
| | | | TTAATCTTCGGTGGCAAGAGGTGTGCAAACAATTGTCGGATAGGAAG | NEFVLWLEEADNIASIP |
| | | | AAACGGCTCGAAGAGCAGAAGAATATACTGTCCGAGTTTCAGCGGGA | LEPGKEQQLKEKLEQV |
| | | | TCTCAATGAGTTTGTTCTCTGGTTAGAAGAGGCGGACAACATTGCTTC | KLLVEELPLRQGILKQL |
| | | | AATACCCCTGGAGCCCGGGAAAGAACAGCAGTTAAAGGAGAAACTC | NETGGPVLVSAPISPEE |
| | | | GAACAAGTGAAACTTCTCGTTGAAGAGCTCCCACTCAGACAAGGGAT | QDKLENKLKQTNLQWI |
| | | | TCTGAAGCAACTGAATGAGACTGGCGGGCCCGTTCTGGTTTCTGCTCC | KVSRALPEKQGEIEAQI |
| | | | CATCTCTCCCGAGGAGCAAGATAAGCTGGAAAACAAGCTGAAACAG | KDLGQLEKKLEDLEEQ |
| | | | ACTAATCTTCAGTGGATTAAAGTCAGCAGGGCCCTTCCCGAGAAACA | LNHLLLWLSPIRNQLEI |
| | | | AGGAGAAATCGAAGCGCAAATAAAAGATCTCGGGCAGTTAGAGAAG | YNQPNQEGPFDVKETE |
| | | | AAATTGGAGGACTTAGAGGAGCAGTTGAATCACCTGTTACTGTGGCT | IAVQAKQPDVEEILSKG |
| | | | CTCCCCGATTAGAAACCAACTGGAGATCTACAATCAGCCGAATCAAG | QHLYKEKPATQPVKRK |
| | | | AGGGGCCCTTTGACGTTAAAGAGACTGAGATTGCCGTTCAAGCGAAA | LEDLSSEWKAVNRLLQ |
| | | | CAGCCCGACGTAGAGGAGATTTTGTCAAAGGGGCAACACCTGTATAA | ELRAKQPDLAPGLTTIG |
| | | | AGAGAAACCCGCTACTCAACCCGTTAAACGGAAACTAGAGGACCTAT | ASPTQTVTLVTQPVVT |
| | | | CTAGTGAATGGAAAGCCGTTAATCGCCTCCTGCAAGAACTCCGGGCC | KETAISKLEMPSSLMLE |
| | | | AAACAACCGGATTTGGCGCCCGGATTAACCACTATAGGTGCCTCTCC | VPALADFNRAWTELTD |
| | | | GACTCAAACTGTTACCCTCGTTACTCAACCGGTTGTTACCAAAGAAAC | WLSLLDQVIKSQRVMV |
| | | | TGCCATTTCTAAACTGGAGATGCCCTCTTCTCTTATGTTAGAGGTTCCC | GDLEDINEMIIKQKAT |
| | | | GCACTGGCCGATTTTAATCGGGCCTGGACCGAGTTAACTGATTGGCTT | MQDLEQRRPQLEELIT |
| | | | TCTCTGTTAGACCAAGTCATCAAATCCCAACGGGTTATGGTAGGGGAT | AAQNLKNKTSNQEAR |
| | | | TTGGAAGACATTAACGAAATGATCATTAAACAGAAAGCCACTATGCA | TIITDRIERIQNQWDEV |
| | | | GGATCTCGAGCAACGGAGACCCCAGCTTGAGGAGCTTATAACCGCCG | QEHLQNRRQQLNEML |
| | | | CCCAGAATCTGAAGAATAAAACGTCGAATCAGGAGGCCCGAACTATC | KDSTQWLEAKEEAEQ |
| | | | ATCACTGATAGAATAGAGCGGATTCAGAATCAATGGGATGAAGTTCA | VLGQARAKLESWKEG |
| | | | GGAGCACTTACAGAATAGGCGGCAACAACTCAACGAAATGCTAAAG | PYTVDAIQKKITETKQL |
| | | | GATAGCACCCAATGGCTCGAAGCTAAGGAAGAGGCCGAGCAAGTTTT | AKDLRQWQTNVDVAN |
| | | | GGGGCAAGCTCGCGCCAAATTAGAGTCTTGGAAGGAGGGGCCTTACA | DLALKLLRDYSADDTR |
| | | | CCGTTGATGCGATCCAGAAGAAGATTACAGAGACTAAACAGCTAGCG | KVHMITENINASWRSI |
| | | | AAAGATCTGCGGCAGTGGCAAACCAATGTTGATGTTGCCAATGACTT | HKRVSEREAALEETHR |
| | | | AGCGCTTAAACTCCTCCGGGACTACTCCGCCGATGATACACGGAAAG | LLQQFPLDLEKFLAWL |
| | | | TGCACATGATAACCGAAAACATCAATGCCTCTTGGCGGTCTATACAC | TEAETTANVLQDATRK |
| | | | AAACGGGTTTCAGAACGAGAGGCAGCCCTTGAAGAGACTCACAGACT | ERLLEDSKGVKELMKQ |
| | | | TCTCCAACAATTTCCCTTGGATCTGGAGAAATTCTTAGCATGGCTTAC | WQDLQGEIEAHTDVY |
| | | | TGAGGCCGAGACCACTGCCAATGTTTTACAAGACGCCACACGGAAAG | HNLDENSQKILRSLEGS |
| | | | AACGGCTCCTAGAGGACTCTAAAGGGGTGAAAGAACTCATGAAACA | DDAVLLQRRLDNMNF |
| | | | GTGGCAGGACTTACAGGGCGAAATAGAGGCCCACACTGATGTTTATC | KWSELRKKSLNIRSHL |
| | | | ACAACCTCGACGAAAACTCTCAGAAAATCCTTAGGTCTCTTGAGGGG | EASSDQWKRLHLSLQE |
| | | | TCTGATGATGCCGTGCTTCTGCAGAGAAGGCTGGACAACATGAACTT | LLVWLQLKDDELSRQ |
| | | | CAAGTGGTCTGAGCTTAGGAAGAAATCGCTCAACATACGGTCCCACC | APIGGDFPAVQKQNDV |
| | | | TAGAGGCCTCTTCTGATCAATGGAAACGGTTGCATTTATCTCTGCAGG | HRAFKRELKTKEPVIM |
| | | | AGTTATTAGTATGGCTCCAATTAAAAGACGACGAGTTGTCTCGGCAG | STLETVRIFLTEQPLEG |
| | | | GCGCCCATTGGTGGGGATTTTCCCGCCGTTCAGAAACAGAACGATGTT | LEKLYQEPRELPPEERA |
| | | | CACCGGGCCTTCAAACGAGAGCTTAAAACAAAAGAGCCCGTTATTAT | QNVTRLLRKQAEEVNT |
| | | | GTCTACCCTCGAAACTGTTCGGATATTCCTCACAGAACAACCCCTCGA | EWEKLNLHSADWQRK |
| | | | AGGGTTAGAGAAACTGTACCAGGAGCCTCGGGAGCTTCCACCGGAGG | IDETLERLRELQEATDE |
| | | | AAAGGGCTCAAAACGTTACTCGGTTATTAAGGAAACAGGCCGAGGAG | LDLKLRQAEVIKGSWQ |
| | | | GTCAATACCGAGTGGGAGAAACTGAATTTGCACTCCGCCGACTGGCA | PVGDLLIDSLQDHLEK |
| | | | ACGGAAAATTGATGAGACTCTCGAGAGACTTCAAGAGCTTCAAGAGG | VKALRGELAPLKENVS |
| | | | CTACAGACGAGCTAGATTTGAAATTGCGCCAAGCCGAAGTTATTAAG | HVNDLARQLTTLGIQL |
| | | | GGGTCTTGGCAACCCGTTGGTGATCTTCTCATTGATTCTCTGCAAGAT | SPYNLSTLEDLNTRWK |
| | | | CACCTAGAGAAAGTTAAAGCCCTCCGGGGTGAGATTGCGCCCCTGAA | LLQVAVEDRVRQLHE |
| | | | AGAGAATGTATCACACGTGAATGATCTTGCCCGGCAACTTACTACTTT | AHRDFGPASQHFLSTS |
| | | | AGGCATTCAACTTTCACCGTATAATTTGTCCACCCTCGAGGATCTAAA | VQGPWERAISPNKVPY |
| | | | TACTCGGTGGAAACTCTTACAAGTAGCCGTTGAGGATAGAGTTCGGC | YINHETQTTCWDHPK |
| | | | AATTACATGAGGCTCACAGAGATTTTGGGCCTGCCTCTCAGCACTTCT | MTELYQSLADLNNVRF |
| | | | TATCTACCTCTGTTCAAGGGCCCTGGGAAAGGGCAATAAGCCCCAAT | SAYRTAMKLRRLQKA |
| | | | AAAGTGCCCTACTACATTAATCACGAGACTCAAACTACTTGCTGGGA | LCLDLLSLSAACDALD |
| | | | CCACCCCAAAATGACGGAATTATACCAGTCTCTTGCCGATTTAAACAA | QHNLKQNDQPMDILQI |
| | | | TGTCCGGTTTTCTGCCTATCGGACAGCCATGAAACTGCGGAGACTTCA | INCLTTIYDRLEQEHNN |
| | | | GAAAGCCTTATGCCTCGATCTTCTTTCACTTTCTGCTGCCTGTGACGCC | LVNVPLCVDMCLNWL |
| | | | CTGGACCAACACAACCTAAAACAAAATGATCAACCCATGGACATTTT | LNVYDTGRTGRIRVLS |
| | | | GCAGATTATAAACTGCCTCACAACTATTTACGACCGGTTAGAACAGG | FKTGIISLCKAHLEDKY |
| | | | AGCATAACAACTTGGTAAACGTACCCCTTTGCGTTGATATGTGCCTAA | RYLFKQVASSTGFCDQ |
| | | | ACTGGCTCCTGAACGTATACGATACAGGACGGACTGGGAGGATTCGG | RRLGLLLHDSIQIPRQL |
| | | | GTTCTAAGCTTTAAGACCGGGATTATTTCTCTGTGCAAGGCCCATCTG | GEVASFGGSNIEPSVRS |
| | | | GAGGACAAATACAGGTATCTCTTCAAACAAGTGGCCTCTAGTACTGG | CFQFANNKPEIEAALFL |
| | | | GTTTTGCGATCAAAGAAGGTTGGGGTTACTGTTACACGACTCTATTCA | DWMRLEPQSMVWLPV |
| | | | AATTCCCCGGCAATTAGGGGAAGTTGCCTCATTTGGTGGGTCTAATAT | LHRVAAAETAKHQAK |
| | | | TGAGCCCTCTGTAAGATCTTGCTTCCAATTCGCCAACAATAAACCCGA | CNICKECPIIGFRYRSLK |
| | | | AATCGAGGCCGCCTTATTTCTCGATTGGATGCGGTTAGAACCCCAATC | HFNYDICQSCFFSGRV |
| | | | TATGGTTTGGTTACCCGTGCTTCACAGAGTTGCGGCAGCTGAGACAGC | AKGHKMHYPMVEYCT |
| | | | CAAACATCAAGCGAAATGCAACATATGCAAAGAGTGTCCCATAATAG | PTTSGEDVRDFAKVLK |
| | | | GGTTTCGGTATCGGTCGTTGAAGCATTTTAACTACGACATATGCCAGT | NKFRTKRYFAKHPRM |
| | | | CCTGCTTCTTTTCAGGGAGAGTTGCCAAAGGGCACAAAATGCATTATC | GYLPVQTVLEGDNME |
| | | | CCATGGTTGAGTATTGCACACCCACTACTTCTGGGGAGGATGTTCGGG | TPVTLINFWPVDSAPAS |
| | | | ATTTTGCTAAAGTTCTGAAGAATAAGTTCCGGACTAAACGGTATTTTG | SPQLSHDDTHSRIEHYA |
| | | | CCAAGCATCCCAGAATGGGGTATTTACCCGTTCAGACAGTTCTGGAG | SRLAEMENSNGSYLND |
| | | | GGCGATAATATGGAGACCCCGGTGACTCTGATCAATTTCTGGCCCGTT | SISPNESIDDEHLLIQHY |
| | | | GATAGTGCACCTGCCTCTAGCCCGCAGCTGTCTCACGACGACACCCAC | CQSLNQDSPLSQPRSPA |
| | | | TCTCGGATAGAGCACTATGCCTCCAGGTTGGCCGAGATGGAGAATAG | QILISLESEERGELERIL |
| | | | TAATGGGTCTTACCTGAACGATTCTATTTCACCCAATGAGTCAATAGA | ADLEEENRNLQAEYDR |
| | | | CGACGAGCATCTACTCATTCAGCATTACTGCCAGTCTCTCAATCAAGA | LKQQHEHKGLSPLPSPP |
| | | | TTCCCCACTGTCTCAGCCCAGATCTCCCGCCCAGATTTTGATTTCACTC | EMMPTSPQSPRDAELI |
| | | | GAGTCTGAAGAGAGAGGGGAGCTCGAAAGGATTTTAGCCGACCTCGA | AEAKLLRQHKGRLEAR |
| | | | GGAGGAGAATCGGAATTTACAAGCCGAGTACGATCGCCTAAAACAAC | MQILEDHNKQLESQLH |
| | | | AACACGAGCACAAAGGGCTGTCTCCCTTACCGTCTCCGCCCGAGATG | RLRQLLEQPQAEAKVN |
| | | | ATGCCCACTTCTCCCCAATCACCCAGAGATGCCGAGCTTATAGCCGAG | GTTVSSPSTSLQRSDSS |
| | | | GCCAAACTCCTTCGGCAACATAAAGGGCGGCTCGAAGCGAGAATGCA | QPMLLRVVGSQTSDSM |
| | | | AATTCTTGAGGATCACAATAAGCAACTCGAGTCTCAGCTTCACAGATT | GEEDLLSPPQDTSTGLE |
| | | | AAGACAGCTCCTTGAACAACCCCAGGCCGAGGCTAAAGTTAATGGGA | EVMEQLNNSFPSSRGR |
| | | | CTACTGTTTCGTCTCCGTCTACTTCATTACAGAGATCTGATTCTTCCCA | NTPGKPMREDTM (SEQ |
| | | | ACCCATGTTACTGAGAGTTGTTGGCAGCCAAACTTCTGATTCTATGGG | ID NO: 6) |
| | | | TGAAGAAGACTTGTTATCTCCGCCGCAGGATACCTCCACTGGGCTAG | |
| | | | AAGAGGTTATGGAGCAGCTTAACAATTCCTTTCCCTCTTCTAGGGGCA | |
| | | | GAAATACACCCGGTAAGCCGATGCGGGAGGACACCATG (SEQ ID NO: | |
| | | | 5) | |
| Full | Wild Type | NP_003997 | ATGCTCTGGTGGGAAGAGGTCGAGGACTGCTATGAGCGGGAGGACGT | MLWWEEVEDCYERED |
| Length | protein | | CCAGAAGAAGACCTTTACGAAATGGGTGAATGCCCAATTCTCTAAAT | VQKKTFTKWVNAQFS |
| human | (CO | | TCGGCAAGCAACATATCGAGAATCTGTTTAGCGACTTACAAGATGGG | KFGKQHIENLFSDLQD |
| dystrophin | nucleotide | | CGGCGGTTACTGGACTTACTGGAGGGGCTGACTGGGCAGAAACTGCC | GRRLLDLLEGLTGQKL |
| | seq-1B) | | GAAGGAGAAAGGGTCCACTAGAGTTCACGCACTTAACAACGTTAACA | PKEKGSTRVHALNNVN |
| | | | AAGCCCTGCGGGTTCTCCAGAACAACAATGTCGACTTAGTTAATATA | KALRVLQNNNVDLVNI |
| | | | GGGTCAACAGACATTGTAGATGGGAATCACAAACTTACTCTGGGGCT | GSTDIVDGNHKLTLGLI |
| | | | CATTTGGAATATAATTCTTCACTGGCAAGTTAAGAACGTCATGAAGA | WNIILHWQVKNVMKN |
| | | | ACATTATGGCCGGGCTCCAACAGACTAACTCGGAGAAAATACTGCTC | IMAGLQQTNSEKILLS |
| | | | TCTTGGGTTAGACAGTCTACCCGGAATTATCCCCAGGTGAATGTGATT | WVRQSTRNYPQVNVIN |
| | | | AATTTTACCACGTCCTGGTCTGATGGGCTTGCCCTTAATGCCCTGATTC | FTTSWSDGLALNALIH |
| | | | ACTCTCATAGACCCGACCTCTTTGACTGGAACAGTGTAGTTTGTCAAC | SHRPDLFDWNSVVCQ |
| | | | AATCTGCCACCCAGAGACTTGAACACGCGTTTAACATTGCGAGATAC | QSATQRLEHAFNIARY |
| | | | CAACTAGGGATAGAGAAATTGCTTGACCCCGAGGATGTTGACACTAC | QLGIEKLLDPEDVDTT |
| | | | TTATCCCGACAAGAAATCCATACTGATGTACATTACCAGTCTATTCCA | YPDKKSILMYITSLFQV |
| | | | AGTTCTTCCCCAACAAGTTTCTATTGAGGCCATTCAGGAGGTGGAGAT | LPQQVSIEAIQEVEMLP |
| | | | GCTACCCCGGCCTCCCAAAGTTACCAAAGAGGAGCATTTTCAACTGC | RPPKVTKEEHFQLHHQ |
| | | | ACCACCAGATGCACTATTCTCAGCAGATAACGGTGTCCCTGGCCCAA | MHYSQQITVSLAQGYE |
| | | | GGGTATGAGAGAACTTCTTCTCCCAAACCCCGGTTCAAATCCTATGCC | RTSSPKPRFKSYAYTQ |
| | | | TATACACAAGCCGCGTATGTTACAACCTCTGATCCCACTCGGTCTCCC | AAYVTTSDPTRSPFPSQ |
| | | | TTTCCCTCTCAACACCTCGAAGCGCCCGAGGATAAATCCTTTGGGTCT | HLEAPEDKSFGSSLME |
| | | | TCTTTAATGGAGTCTGAGGTCAACCTCGATCGGTATCAAACTGCGCTT | SEVNLDRYQTALEEVL |
| | | | GAAGAGGTGTTATCCTGGCTTTTGTCTGCCGAGGATACTCTTCAAGCC | SWLLSAEDTLQAQGEI |
| | | | CAAGGGGAGATTTCTAATGATGTCGAGGTGGTCAAAGATCAGTTTCA | SNDVEVVKDQFHTHE |
| | | | CACCCACGAGGGGTACATGATGGATTTGACTGCCCACCAAGGACGGG | GYMMDLTAHQGRVG |
| | | | TTGGGAATATACTCCAGCTCGGGTCTAAACTCATCGGGACAGGGAAA | NILQLGSKLIGTGKLSE |
| | | | CTCAGCGAGGACGAGGAAACAGAGGTCCAGGAACAGATGAATCTCC | DEETEVQEQMNLLNSR |
| | | | TGAATTCACGTTGGGAGTGTCTTAGAGTTGCCTCTATGGAGAAACAAT | WECLRVASMEKQSNL |
| | | | CTAACCTACATCGGGTTTTAATGGATCTCCAGAATCAGAAGCTCAAG | HRVLMDLQNQKLKEL |
| | | | GAGCTAAATGATTGGCTAACCAAAACCGAGGAGAGAACACGGAAAA | NDWLTKTEERTRKME |
| | | | TGGAGGAGGAACCGTTAGGGCCCGACCTAGAGGACTTAAAACGGCA | EEPLGPDLEDLKRQVQ |
| | | | AGTTCAACAACACAAAGTTCTCCAAGAAGATTTAGAGCAGGAGCAAG | QHKVLQEDLEQEQVR |
| | | | TTAGAGTCAATTCACTGACTCACATGGTTGTAGTGGTCGATGAGTCTT | VNSLTHMVVVVDESS |
| | | | CTGGGGACCACGCCACTGCAGCCCTTGAGGAGCAGTTAAAAGTTCTT | GDHATAALEEQLKVL |
| | | | GGGGATAGATGGGCCAATATTTGCCGGTGGACAGAAGATCGGTGGGT | GDRWANICRWTEDRW |
| | | | ACTTCTGCAGGACATTCTACTGAAATGGCAACGGTTAACCGAGGAAC | VLLQDILLKWQRLTEE |
| | | | AATGCTTATTCTCTGCCTGGTTATCTGAGAAAGAAGACGCCGTCAACA | QCLFSAWLSEKEDAVN |
| | | | AGATACACACGACTGGATTCAAAGACCAAAACGAAATGCTGTCATCT | KIHTTGFKDQNEMLSS |
| | | | CTGCAGAAATTGGCCGTGTTAAAAGCCGATCTCGAGAAGAAGAAGCA | LQKLAVLKADLEKKK |
| | | | GTCTATGGGGAAGCTTTATTCGCTGAAACAAGACCTTCTTTCTACCCT | QSMGKLYSLKQDLLST |
| | | | TAAGAATAAAAGTGTGACCCAAAAGACCGAAGCCTGGTTGGATAATT | LKNKSVTQKTEAWLD |
| | | | TTGCCCGGTGCTGGGATAATCTCGTACAAAAGCTCGAGAAATCTACA | NFARCWDNLVQKLEK |
| | | | GCCCAAATTTCCCAAGCCGTTACTACTACTCAACCCTCTCTTACTCAG | STAQISQAVTTTQPSLT |
| | | | ACTACTGTTATGGAGACTGTTACTACTGTTACTACACGGGAGCAAATA | QTTVMETVTTVTTREQ |
| | | | CTCGTAAAACACGCCCAGGAAGAATTACCACCGCCACCGCCCCAGAA | ILVKHAQEELPPPPPQK |
| | | | GAAACGGCAAATTACTGTGGACTCTGAGATTCGGAAAAGATTAGACG | KRQITVDSEIRKRLDVD |
| | | | TCGATATTACCGAGCTTCATTCGTGGATAACTAGATCTGAGGCCGTTC | ITELHSWITRSEAVLQS |
| | | | TTCAGTCTCCCGAGTTTGCTATATTTCGGAAAGAGGGGAACTTTAGTG | PEFAIFRKEGNFSDLKE |
| | | | ATTTAAAGGAGAAAGTAAATGCCATAGAGAGAGAGAAAGCCGAGAA | KVNAIEREKAEKFRKL |
| | | | ATTTCGGAAACTCCAGGATGCCTCACGGTCCGCCCAAGCCTTGGTGG | QDASRSAQALVEQMV |
| | | | AACAAATGGTCAATGAGGGCGTTAATGCCGATTCTATCAAACAAGCC | NEGVNADSIKQASEQL |
| | | | TCCGAACAACTCAATTCTCGGTGGATTGAGTTTTGCCAGTTACTCTCC | NSRWIEFCQLLSERLN |
| | | | GAACGGCTCAATTGGTTAGAGTACCAAAACAATATAATTGCATTCTAT | WLEYQNNIIAFYNQLQ |
| | | | AACCAGCTCCAGCAACTCGAGCAAATGACTACAACTGCGGAGAACTG | QLEQMTTTAENWLKIQ |
| | | | GCTTAAAATACAGCCGACAACTCCGTCTGAGCCCACGGCCATAAAGT | PTTPSEPTAIKSQLKICK |
| | | | CGCAGCTAAAGATATGCAAGGACGAGGTCAATCGGTTATCTGATCTT | DEVNRLSDLQPQIERL |
| | | | CAACCCCAGATTGAGCGGCTCAAAATACAGTCTATAGCCCTGAAAGA | KIQSIALKEKGQGPMFL |
| | | | GAAGGGACAAGGGCCCATGTTTCTTGATGCCGACTTCGTAGCTTTTAC | DADFVAFTNHFKQVFS |
| | | | AAACCATTTCAAGCAAGTCTTTTCTGACGTTCAAGCGAGAGAAAAGG | DVQAREKELQTIFDTLP |
| | | | AGCTGCAGACCATTTTCGACACTCTTCCACCCATGCGGTATCAAGAGA | PMRYQETMSAIRTWV |
| | | | CCATGTCTGCCATTCGGACCTGGGTACAACAATCTGAGACTAAACTCT | QQSETKLSIPQLSVTDY |
| | | | CTATCCCGCAGCTATCCGTGACCGACTATGAAATAATGGAGCAACGT | EIMEQRLGELQALQSS |
| | | | CTCGGGGAGCTCCAAGCCCTTCAAAGCTCTCTACAAGAGCAACAGTC | LQEQQSGLYYLSTTVK |
| | | | CGGGTTATATTATCTCAGCACCACCGTAAAAGAGATGTCTAAGAAAG | EMSKKAPSEISRKYQSE |
| | | | CTCCCTCCGAGATTTCGAGAAAATACCAGTCTGAGTTTGAGGAGATTG | FEEIEGRWKKLSSQLV |
| | | | AAGGGAGATGGAAGAAATTATCGTCTCAACTCGTGGAGCACTGCCAG | EHCQKLEEQMNKLRKI |
| | | | AAACTGGAGGAGCAGATGAATAAGTTACGGAAAATTCAGAACCATAT | QNHIQTLKKWMAEVD |
| | | | CCAAACCCTGAAGAAATGGATGGCCGAGGTTGACGTGTTCTTAAAAG | VFLKEEWPALGDSEIL |
| | | | AAGAGTGGCCTGCCCTAGGAGACTCAGAGATCCTTAAGAAGCAACTT | KKQLKQCRLLVSDIQTI |
| | | | AAGCAATGCCGGCTGTTAGTTTCCGACATTCAAACTATCCAGCCCTCC | QPSLNSVNEGGQKIKN |
| | | | CTAAACAGCGTTAATGAAGGCGGGCAGAAAATCAAGAACGAGGCTG | EAEPEFASRLETELKEL |
| | | | AGCCCGAATTTGCCTCTCGGCTCGAGACTGAGCTCAAAGAGCTTAAT | NTQWDHMCQQVYAR |
| | | | ACACAGTGGGACCATATGTGCCAACAAGTATATGCCCGCAAAGAGGC | KEALKGGLEKTVSLQK |
| | | | ACTTAAAGGTGGGCTTGAGAAAACCGTTTCTCTGCAAAAGGATCTTTC | DLSEMHEWMTQAEEE |
| | | | GGAAATGCACGAGTGGATGACTCAAGCAGAGGAGGAGTATCTCGAG | YLERDFEYKTPDELQK |
| | | | CGGGACTTCGAGTATAAAACTCCCGACGAATTACAGAAAGCGGTTGA | AVEEMKRAKEEAQQK |
| | | | AGAGATGAAACGGGCAAAAGAGGAGGCTCAACAGAAAGAGGCCAA | EAKVKLLTESVNSVIA |
| | | | AGTGAAACTGTTAACCGAGTCGGTTAATTCTGTGATTGCCCAGGCTCC | QAPPVAQEALKKELET |
| | | | ACCCGTTGCCCAGGAAGCCCTTAAGAAAGAACTCGAGACACTCACTA | LTTNYQWLCTRLNGK |
| | | | CGAACTATCAATGGCTCTGCACTCGGCTTAATGGCAAGTGTAAAACG | CKTLEEVWACWHELL |
| | | | TTGGAGGAGGTTTGGGCCTGCTGGCACGAACTACTCTCTTATCTCGAA | SYLEKANKWLNEVEF |
| | | | AAGGCGAACAAATGGCTGAATGAGGTGGAATTCAAACTCAAAACCA | KLKTTENIPGGAEEISE |
| | | | CTGAAAATATTCCCGGTGGTGCCGAGGAAATTTCTGAAGTGTTGGACT | VLDSLENLMRHSEDNP |
| | | | CACTTGAGAATCTGATGAGGCATTCTGAGGACAATCCCAATCAGATA | NQIRILAQTLTDGGVM |
| | | | CGGATTCTTGCCCAAACTCTCACTGATGGCGGGGTTATGGATGAGCTG | DELINEELETFNSRWRE |
| | | | ATTAATGAAGAGCTAGAGACCTTCAATTCAAGATGGCGGGAGCTCCA | LHEEAVRRQKLLEQSI |
| | | | CGAAGAGGCCGTTAGGCGGCAAAAGCTCCTCGAACAGTCTATTCAAT | QSAQETEKSLHLIQESL |
| | | | CTGCCCAAGAGACTGAGAAAAGCTTACACCTTATCCAAGAGTCTCTC | TFIDKQLAAYIADKVD |
| | | | ACTTTCATCGACAAGCAATTGGCCGCCTACATAGCGGATAAAGTAGA | AAQMPQEAQKIQSDLT |
| | | | CGCCGCCCAAATGCCCCAAGAAGCGCAGAAGATCCAATCCGATTTGA | SHEISLEEMKKHNQGK |
| | | | CTTCCCACGAGATTTCCCTAGAGGAGATGAAGAAACACAATCAGGGG | EAAQRVLSQIDVAQKK |
| | | | AAAGAGGCAGCCCAACGGGTTCTTTCCCAAATTGATGTTGCCCAGAA | LQDVSMKFRLFQKPAN |
| | | | GAAGCTCCAAGATGTAAGCATGAAGTTCCGTCTATTTCAAAAGCCAG | FEQRLQESKMILDEVK |
| | | | CCAATTTTGAGCAACGGTTACAAGAGTCTAAGATGATCCTTGATGAG | MHLPALETKSVEQEVV |
| | | | GTGAAAATGCACCTTCCCGCGCTCGAGACCAAATCTGTTGAACAAGA | QSQLNHCVNLYKSLSE |
| | | | GGTTGTCCAATCGCAATTGAATCACTGTGTGAACCTGTATAAGAGTCT | VKSEVEMVIKTGRQIV |
| | | | TAGCGAGGTTAAATCTGAGGTCGAGATGGTTATTAAAACCGGGCGGC | QKKQTENPKELDERVT |
| | | | AAATTGTTCAAAAGAAACAGACAGAGAATCCCAAAGAGCTTGACGA | ALKLHYNELGAKVTER |
| | | | GCGGGTTACTGCCTTGAAGCTACACTACAATGAACTAGGGGCCAAAG | KQQLEKCLKLSRKMR |
| | | | TAACCGAGAGAAAACAGCAACTGGAGAAATGTCTCAAACTGTCCCGG | KEMNVLTEWLAATDM |
| | | | AAAATGCGGAAGGAGATGAATGTTTTGACCGAGTGGCTTGCCGCCAC | ELTKRSAVEGMPSNLD |
| | | | TGACATGGAGCTTACTAAACGGTCTGCCGTAGAGGGGATGCCCTCTA | SEVAWGKATQKEIEKQ |
| | | | ATCTTGATTCCGAGGTTGCCTGGGGTAAAGCCACCCAGAAGGAAATC | KVHLKSITEVGEALKT |
| | | | GAAAAGCAGAAAGTACATCTTAAAAGTATAACCGAAGTTGGGGAGG | VLGKKETLVEDKLSLL |
| | | | CCCTGAAAACTGTTCTTGGGAAGAAAGAGACCCTCGTGGAGGACAAA | NSNWIAVTSRAEEWLN |
| | | | CTGAGTCTGCTTAACAGTAACTGGATAGCCGTTACTTCTCGTGCCGAG | LLLEYQKHMETFDQN |
| | | | GAGTGGCTTAACCTTTTACTCGAGTATCAGAAACACATGGAGACATTT | VDHITKWIIQADTLLDE |
| | | | GACCAGAATGTTGACCACATCACCAAGTGGATAATTCAGGCCGACAC | SEKKKPQQKEDVLKRL |
| | | | TTTATTAGACGAGTCTGAAAAGAAGAAACCCCAACAAAAGGAAGAC | KAELNDIRPKVDSTRD |
| | | | GTGTTAAAACGGCTGAAAGCCGAGCTCAATGACATACGGCCCAAAGT | QAANLMANRGDHCRK |
| | | | AGATTCTACTCGGGATCAGGCCGCCAATCTGATGGCCAATAGAGGGG | LVEPQISELNHRFAAIS |
| | | | ATCACTGCAGAAAACTCGTTGAGCCCCAAATATCTGAGCTCAATCAC | HRIKTGKASIPLKELEQ |
| | | | AGATTTGCCGCCATTTCACACCGGATCAAAACTGGGAAAGCCTCGAT | FNSDIQKLLEPLEAEIQ |
| | | | ACCCTTAAAAGAGCTGGAACAGTTTAACTCTGACATTCAGAAACTTCT | QGVNLKEEDFNKDMN |
| | | | TGAGCCCCTTGAGGCCGAGATTCAACAAGGGGTTAATCTTAAAGAGG | EDNEGTVKELLQRGDN |
| | | | AGGACTTCAACAAAGACATGAATGAGGATAATGAGGGGACCGTGAA | LQQRITDERKREEIKIK |
| | | | AGAACTACTCCAGAGGGGTGACAATCTCCAACAAAGGATTACTGATG | QQLLQTKHNALKDLRS |
| | | | AGCGGAAAAGAGAGGAAATCAAAATTAAGCAACAGTTATTGCAAAC | QRRKKALEISHQWYQ |
| | | | CAAGCATAATGCTCTCAAAGACCTTCGGTCTCAACGCCGGAAGAAAG | YKRQADDLLKCLDDIE |
| | | | CGTTGGAAATATCTCACCAGTGGTACCAATATAAACGCCAAGCCGAC | KKLASLPEPRDERKIKE |
| | | | GATCTGTTAAAATGCCTGGACGATATAGAGAAGAAACTCGCGTCTCT | IDRELQKKKEELNAVR |
| | | | CCCCGAACCCAGAGATGAGCGCAAGATCAAAGAAATCGACCGGGAG | RQAEGLSEDGAAMAV |
| | | | CTTCAGAAGAAGAAAGAGGAGCTGAATGCCGTTCGCAGACAAGCCG | EPTQIQLSKRWREIESK |
| | | | AGGGCCTTTCTGAGGATGGGGCTGCCATGGCCGTTGAACCCACTCAA | FAQFRRLNFAQIHTVR |
| | | | ATACAACTTTCTAAACGGTGGAGAGAGATCGAGTCAAAATTCGCCCA | EETMMVMTEDMPLEIS |
| | | | ATTTCGGCGGCTCAATTTCGCCCAAATACACACAGTTCGGGAGGAGA | YVPSTYLTEITHVSQAL |
| | | | CTATGATGGTTATGACTGAGGATATGCCCCTGGAGATTTCTTATGTTC | LEVEQLLNAPDLCAKD |
| | | | CCTCTACCTACCTAACTGAAATTACCCACGTTTCTCAGGCCTTGCTTG | FEDLFKQEESLKNIKDS |
| | | | AGGTTGAGCAGTTACTCAATGCGCCCGATCTCTGTGCCAAAGACTTTG | LQQSSGRIDIIHSKKTA |
| | | | AGGATCTCTTCAAACAAGAGGAGAGTCTAAAGAATATTAAAGACAGC | ALQSATPVERVKLQEA |
| | | | CTCCAGCAATCTTCTGGGCGGATCGACATTATTCACAGCAAGAAAAC | LSQLDFQWEKVNKMY |
| | | | TGCTGCTCTGCAATCTGCTACTCCCGTTGAGCGGGTTAAACTCCAAGA | KDRQGRFDRSVEKWR |
| | | | GGCTCTCTCGCAATTAGATTTCCAATGGGAAAAGGTAAACAAAATGT | RFHYDIKIFNQWLTEA |
| | | | ATAAAGATCGGCAAGGGAGATTTGATCGGTCTGTTGAGAAATGGCGG | EQFLRKTQIPENWEHA |
| | | | AGATTTCATTATGACATTAAAATCTTCAATCAGTGGTTAACGGAGGCC | KYKWYLKELQDGIGQ |
| | | | GAGCAATTTCTTCGGAAAACTCAAATTCCGGAGAATTGGGAACATGC | RQTVVRTLNATGEEIIQ |
| | | | CAAGTACAAGTGGTATCTTAAAGAACTTCAGGATGGGATAGGGCAAA | QSSKTDASILQEKLGSL |
| | | | GACAAACTGTGGTTCGGACATTAAATGCCACTGGCGAGGAAATTATT | NLRWQEVCKQLSDRK |
| | | | CAGCAGTCATCCAAAACCGATGCCTCCATATTACAGGAGAAACTAGG | KRLEEQKNILSEFQRDL |
| | | | GTCTCTTAATCTCCGGTGGCAAGAGGTGTGCAAACAACTGTCAGATA | NEFVLWLEEADNIASIP |
| | | | GAAAGAAACGGTTAGAGGAGCAGAAGAATATACTCTCGGAGTTTCAA | LEPGKEQQLKEKLEQV |
| | | | AGGGATCTAAACGAATTCGTCCTTTGGTTAGAGGAAGCTGACAATAT | KLLVEELPLRQGILKQL |
| | | | TGCTAGCATTCCCCTCGAGCCGGGGAAAGAACAGCAGCTGAAGGAGA | NETGGPVLVSAPISPEE |
| | | | AACTGGAACAGGTGAAATTACTTGTTGAGGAGCTGCCCCTCAGACAA | QDKLENKLKQTNLQWI |
| | | | GGGATTCTCAAACAACTGAATGAGACTGGTGGGCCCGTTCTTGTTTCT | KVSRALPEKQGEIEAQI |
| | | | GCCCCTATTTCTCCGGAGGAGCAGGATAAACTCGAGAATAAACTGAA | KDLGQLEKKLEDLEEQ |
| | | | ACAGACCAACCTCCAATGGATAAAAGTATCCCGGGCCTTACCCGAGA | LNHLLLWLSPIRNQLEI |
| | | | AACAGGGTGAAATTGAGGCGCAAATAAAAGACCTGGGCCAACTTGA | YNQPNQEGPFDVKETE |
| | | | GAAGAAATTGGAGGATCTCGAAGAGCAACTGAACCACCTCTTACTAT | IAVQAKQPDVEEILSKG |
| | | | GGCTGTCTCCCATTAGGAACCAGCTGGAAATCTATAATCAGCCGAAT | QHLYKEKPATQPVKRK |
| | | | CAAGAGGGGCCGTTTGATGTTAAAGAAACCGAGATAGCCGTACAAGC | LEDLSSEWKAVNRLLQ |
| | | | CAAGCAACCCGATGTCGAGGAGATACTCTCTAAAGGGCAACATCTCT | ELRAKQPDLAPGLTTIG |
| | | | ACAAGGAAAAGCCTGCCACTCAGCCCGTCAAAAGGAAACTAGAGGA | ASPTQTVTLVTQPVVT |
| | | | TCTGTCTTCTGAATGGAAAGCCGTAAATCGGCTCCTTCAGGAATTACG | KETAISKLEMPSSLMLE |
| | | | GGCTAAACAACCCGATCTTGCCCCGGGATTGACTACTATTGGGGCCTC | VPALADFNRAWTELTD |
| | | | GCCCACTCAAACCGTTACTTTAGTGACTCAACCCGTTGTAACTAAAGA | WLSLLDQVIKSQRVMV |
| | | | GACTGCCATTTCCAAACTTGAGATGCCCTCTTCTTTGATGTTAGAGGT | GDLEDINEMIIKQKAT |
| | | | TCCCGCACTCGCCGATTTTAATAGAGCCTGGACCGAGCTTACTGACTG | MQDLEQRRPQLEELIT |
| | | | GCTCTCTTTACTCGATCAAGTCATCAAATCCCAGAGAGTGATGGTTGG | AAQNLKNKTSNQEAR |
| | | | GGATCTGGAGGATATTAACGAGATGATAATTAAGCAGAAAGCGACA | TIITDRIERIQNQWDEV |
| | | | ATGCAGGATTTAGAACAACGGAGACCGCAATTAGAGGAGCTCATTAC | QEHLQNRRQQLNEML |
| | | | TGCGGCCCAAAACTTAAAGAACAAGACCAGCAATCAAGAGGCCAGA | KDSTQWLEAKEEAEQ |
| | | | ACGATAATCACTGACCGAATTGAGAGAATCCAGAATCAATGGGATGA | VLGQARAKLESWKEG |
| | | | GGTGCAGGAACACCTCCAGAATCGGAGACAACAGTTGAACGAGATG | PYTVDAIQKKITETKQL |
| | | | CTCAAAGATTCCACTCAGTGGCTTGAAGCGAAAGAGGAAGCGGAACA | AKDLRQWQTNVDVAN |
| | | | GGTCCTTGGCCAAGCCAGAGCCAAACTGGAGTCCTGGAAAGAGGGCC | DLALKLLRDYSADDTR |
| | | | CCTATACTGTGGATGCGATCCAGAAGAAAATAACCGAAACAAAACAG | KVHMITENINASWRSI |
| | | | TTAGCGAAAGATCTGAGACAATGGCAAACTAACGTAGATGTTGCCAA | HKRVSEREAALEETHR |
| | | | TGACCTTGCCCTGAAACTACTCCGGGATTATTCCGCCGACGATACAAG | LLQQFPLDLEKFLAWL |
| | | | AAAGGTTCACATGATCACAGAGAACATAAATGCCTCTTGGCGGTCTA | TEAETTANVLQDATRK |
| | | | TACACAAACGGGTATCTGAAAGGGAGGCCGCTCTCGAAGAAACCCAC | ERLLEDSKGVKELMKQ |
| | | | AGACTGCTTCAGCAGTTTCCCCTAGACCTGGAGAAATTTCTTGCCTGG | WQDLQGEIEAHTDVY |
| | | | CTTACCGAAGCCGAAACAACAGCCAATGTTTTACAGGATGCCACACG | HNLDENSQKILRSLEGS |
| | | | GAAAGAGCGGCTACTCGAGGACTCTAAAGGGGTCAAGGAACTGATG | DDAVLLQRRLDNMNF |
| | | | AAACAATGGCAGGATCTTCAAGGTGAAATAGAGGCACACACTGACGT | KWSELRKKSLNIRSHL |
| | | | TTATCATAACCTCGACGAGAATTCACAAAAGATCCTACGGTCCCTTGA | EASSDQWKRLHLSLQE |
| | | | GGGGTCTGATGACGCCGTTCTCCTTCAAAGGCGGTTGGATAACATGA | LLVWLQLKDDELSRQ |
| | | | ATTTTAAATGGTCGGAGTTACGCAAGAAAAGTCTCAACATTCGGTCTC | APIGGDFPAVQKQNDV |
| | | | ATCTGGAGGCCTCCTCTGACCAGTGGAAACGGTTACACTTATCTCTCC | HRAFKRELKTKEPVIM |
| | | | AGGAACTCCTCGTTTGGTTACAACTGAAAGACGATGAGTTGTCTCGGC | STLETVRIFLTEQPLEG |
| | | | AAGCTCCCATAGGCGGGGATTTTCCCGCCGTTCAGAAACAGAACGAC | LEKLYQEPRELPPEERA |
| | | | GTTCACAGGGCCTTTAAGCGGGAGCTTAAAACTAAAGAGCCGGTGAT | QNVTRLLRKQAEEVNT |
| | | | TATGTCCACTCTCGAGACTGTTAGGATATTTCTCACCGAGCAACCGCT | EWEKLNLHSADWQRK |
| | | | TGAGGGCCTTGAGAAACTCTATCAAGAGCCCAGAGAATTACCTCCCG | IDETLERLRELQEATDE |
| | | | AAGAGCGTGCCCAGAATGTTACCAGACTCCTAAGAAAACAAGCGGA | LDLKLRQAEVIKGSWQ |
| | | | GGAGGTTAATACTGAGTGGGAGAAACTGAATCTCCACTCTGCCGACT | PVGDLLIDSLQDHLEK |
| | | | GGCAACGCAAGATTGATGAGACATTGGAGCGGCTCCAAGAATTACAA | VKALRGEIAPLKENVS |
| | | | GAGGCCACCGATGAGCTTGACCTTAAATTACGGCAAGCGGAGGTAAT | HVNDLARQLTTLGIQL |
| | | | AAAAGGTAGCTGGCAACCGGTGGGAGACCTGCTTATCGATTCTTTAC | SPYNLSTLEDLNTRWK |
| | | | AGGATCATCTCGAGAAAGTTAAAGCCCTCCGGGGCGAGATTGCTCCG | LLQVAVEDRVRQLHE |
| | | | CTCAAAGAGAATGTGTCTCACGTGAATGACTTAGCCCGGCAACTCAC | AHRDFGPASQHELSTS |
| | | | TACTTTGGGGATTCAACTGTCTCCCTACAATCTTAGTACTCTGGAGGA | VQGPWERAISPNKVPY |
| | | | CTTGAATACTCGGTGGAAACTTCTGCAAGTTGCTGTTGAGGATAGAGT | YINHETQTTCWDHPK |
| | | | TAGACAGCTTCACGAGGCCCACAGAGACTTTGGGCCCGCTTCCCAAC | MTELYQSLADLNNVRF |
| | | | ACTTTCTCTCTACTTCTGTACAAGGTCCCTGGGAACGAGCCATTTCTCC | SAYRTAMKLRRLQKA |
| | | | GAACAAAGTCCCTTACTATATAAATCATGAGACTCAAACCACTTGCTG | LCLDLLSLSAACDALD |
| | | | GGATCACCCCAAAATGACGGAGCTATATCAATCTCTGGCCGATCTGA | QHNLKQNDQPMDILQI |
| | | | ACAATGTTCGGTTTTCTGCCTACAGAACCGCCATGAAACTGAGACGG | INCLTTIYDRLEQEHNN |
| | | | CTGCAGAAAGCCTTATGCCTCGACCTCCTATCTCTTTCTGCCGCCTGTG | LVNVPLCVDMCLNWL |
| | | | ATGCCTTAGACCAACACAACCTTAAGCAAAATGACCAACCCATGGAC | LNVYDTGRTGRIRVLS |
| | | | ATTCTCCAGATTATCAACTGCCTCACTACGATTTATGACAGACTGGAA | FKTGIISLCKAHLEDKY |
| | | | CAAGAGCACAACAACCTCGTTAATGTTCCCCTCTGCGTCGATATGTGC | RYLFKQVASSTGFCDQ |
| | | | TTGAATTGGCTCCTCAATGTATATGACACCGGGAGAACTGGGAGAAT | RRLGLLLHDSIQIPRQL |
| | | | TCGCGTTCTGTCGTTTAAGACTGGTATCATATCTCTTTGCAAAGCCCA | GEVASFGGSNIEPSVRS |
| | | | CTTAGAGGACAAATATCGGTATCTATTTAAACAGGTTGCCTCTTCAAC | CFQFANNKPEIEAALFL |
| | | | TGGGTTTTGCGATCAACGGCGGCTTGGTCTCCTCCTGCACGACTCTAT | DWMRLEPQSMVWLPV |
| | | | TCAAATACCACGGCAGCTAGGGGAGGTGGCCTCATTTGGCGGTTCTA | LHRVAAAETAKHQAK |
| | | | ATATTGAACCCTCTGTCCGGTCTTGTTTTCAGTTCGCGAACAATAAGC | CNICKECPIIGFRYRSLK |
| | | | CTGAGATAGAGGCTGCCCTATTCTTAGATTGGATGCGGTTAGAGCCCC | HFNYDICQSCFFSGRV |
| | | | AATCTATGGTGTGGCTCCCCGTACTTCATAGAGTTGCGGCGGCGGAG | AKGHKMHYPMVEYCT |
| | | | ACTGCCAAACACCAAGCTAAATGCAACATATGCAAAGAGTGTCCCAT | PTTSGEDVRDFAKVLK |
| | | | TATTGGGTTTCGGTATCGGTCTCTTAAACACTTCAACTATGACATATG | NKFRTKRYFAKHPRM |
| | | | CCAATCCTGCTTCTTTTCTGGGCGGGTTGCCAAAGGGCATAAAATGCA | GYLPVQTVLEGDNME |
| | | | CTATCCCATGGTAGAGTATTGTACTCCCACTACTTCTGGGGAGGACGT | TPVTLINFWPVDSAPAS |
| | | | TAGAGACTTTGCGAAGGTACTCAAGAACAAATTTCGAACGAAACGCT | SPQLSHDDTHSRIEHYA |
| | | | ATTTTGCGAAACACCCTCGGATGGGGTATCTTCCCGTTCAAACTGTGC | SRLAEMENSNGSYLND |
| | | | TAGAGGGCGACAATATGGAGACTCCCGTAACGCTTATCAACTTCTGG | SISPNESIDDEHLLIQHY |
| | | | CCCGTTGATTCTGCGCCCGCCTCTTCTCCACAGCTCTCTCATGATGATA | CQSLNQDSPLSQPRSPA |
| | | | CTCATTCTCGGATAGAGCACTATGCCTCCAGACTTGCCGAGATGGAG | QILISLESEERGELERIL |
| | | | AATTCCAATGGTTCTTACCTGAATGACTCTATTTCCCCAAACGAGTCT | ADLEEENRNLQAEYDR |
| | | | ATTGATGATGAGCACCTCCTTATTCAGCACTATTGCCAGTCTCTAAAT | LKQQHEHKGLSPLPSPP |
| | | | CAAGACTCTCCCCTCTCTCAACCCAGATCTCCGGCCCAGATTTTAATA | EMMPTSPQSPRDAELI |
| | | | TCACTCGAGTCTGAAGAACGCGGCGAGCTGGAGAGAATTCTAGCCGA | AEAKLLRQHKGRLEAR |
| | | | TTTAGAAGAGGAGAATCGGAATCTTCAAGCCGAGTATGACAGGCTGA | MQILEDHNKQLESQLH |
| | | | AACAACAACACGAGCACAAAGGTCTTTCTCCCCTTCCCTCTCCACCCG | RLRQLLEQPQAEAKVN |
| | | | AGATGATGCCCACTTCCCCTCAGTCCCCAAGAGATGCCGAGCTCATTG | GTTVSSPSTSLQRSDSS |
| | | | CTGAGGCCAAACTGTTAAGACAGCACAAAGGGCGGTTAGAAGCCCG | QPMLLRVVGSQTSDSM |
| | | | GATGCAAATTCTCGAGGATCATAATAAACAGCTCGAGTCTCAACTTC | GEEDLLSPPQDTSTGLE |
| | | | ACCGGTTACGGCAGCTGCTTGAGCAGCCGCAAGCCGAAGCCAAAGTT | EVMEQLNNSFPSSRGR |
| | | | AATGGGACAACTGTTTCTTCACCCTCTACCTCTCTCCAGCGGTCTGATT | NTPGKPMREDTM (SEQ |
| | | | CTTCTCAGCCCATGTTATTACGGGTTGTTGGGTCTCAGACATCTGACT | ID NO: 8) |
| | | | CTATGGGCGAGGAGGATCTTCTCTCTCCTCCCCAAGATACTTCTACAG | |
| | | | GCTTAGAGGAGGTTATGGAGCAGCTCAACAATTCTTTTCCCTCTTCCA | |
| | | | GAGGGCGGAATACACCCGGGAAGCCGATGAGAGAGGATACCATG | |
| | | | (SEQ ID NO: 7) | |
| Full | Wild Type | NP_003997 | ATGCTCTGGTGGGAAGAGGTTGAGGACTGCTACGAGCGGGAAGACGT | MLWWEEVEDCYERED |
| Length | protein | | CCAAAAGAAGACATTCACCAAATGGGTCAATGCCCAATTCTCCAAGT | VQKKTFTKWVNAQFS |
| human | (CO | | TCGGTAAGCAGCACATCGAGAATTTGTTCTCTGATCTCCAGGACGGA | KFGKQHIENLFSDLQD |
| dystrophin | nucleotide | | AGACGGCTCCTGGACCTGTTGGAGGGACTGACCGGCCAGAAATTGCC | GRRLLDLLEGLTGQKL |
| | seq-2A) | | CAAAGAAAAGGGCTCTACCCGGGTGCACGCACTGAACAATGTCAACA | PKEKGSTRVHALNNVN |
| | | | AGGCCCTGAGGGTGCTCCAGAACAACAACGTGGACCTCGTGAATATC | KALRVLQNNNVDLVNI |
| | | | GGGAGTACCGACATCGTCGACGGGAATCACAAGCTGACCCTCGGACT | GSTDIVDGNHKLTLGLI |
| | | | GATCTGGAACATCATTCTGCATTGGCAGGTGAAGAACGTCATGAAGA | WNIILHWQVKNVMKN |
| | | | ACATTATGGCCGGTTTGCAGCAGACAAACTCCGAGAAGATCTTGCTG | IMAGLQQTNSEKILLS |
| | | | TCCTGGGTTCGGCAGTCAACCCGGAACTATCCCCAGGTGAACGTCATT | WVRQSTRNYPQVNVIN |
| | | | AACTTTACTACCTCTTGGTCCGACGGCCTTGCCTTGAACGCCCTGATC | FTTSWSDGLALNALIH |
| | | | CACTCCCACAGGCCAGACCTCTTCGACTGGAACAGCGTGGTCTGCCA | SHRPDLFDWNSVVCQ |
| | | | GCAATCTGCCACCCAGCGGCTTGAACACGCCTTCAATATCGCACGCTA | QSATQRLEHAFNIARY |
| | | | CCAGTTGGGCATCGAAAAGCTTCTCGACCCCGAAGACGTGGACACCA | QLGIEKLLDPEDVDTT |
| | | | CCTACCCCGACAAGAAGAGCATCCTGATGTATATCACCTCCCTGTTCC | YPDKKSILMYITSLFQV |
| | | | AGGTGCTCCCTCAACAGGTGAGCATCGAGGCCATCCAGGAAGTCGAG | LPQQVSIEAIQEVEMLP |
| | | | ATGCTGCCACGCCCTCCCAAGGTCACCAAGGAGGAGCACTTCCAACT | RPPKVTKEEHFQLHHQ |
| | | | CCATCACCAGATGCATTATTCTCAGCAGATCACAGTGTCACTGGCCCA | MHYSQQITVSLAQGYE |
| | | | GGGTTACGAGCGGACATCCTCACCCAAGCCCCGGTTCAAGTCCTACG | RTSSPKPRFKSYAYTQ |
| | | | CCTACACCCAGGCTGCTTATGTCACCACCTCAGACCCAACCCGGAGTC | AAYVTTSDPTRSPFPSQ |
| | | | CCTTTCCCTCCCAGCATCTGGAGGCTCCCGAAGACAAGTCCTTTGGAT | HLEAPEDKSFGSSLME |
| | | | CCTCCTTGATGGAGAGCGAAGTCAACCTCGACCGGTACCAGACCGCT | SEVNLDRYQTALEEVL |
| | | | TTGGAAGAGGTGCTCTCCTGGCTCCTGTCCGCAGAAGACACCCTCCAA | SWLLSAEDTLQAQGEI |
| | | | GCCCAGGGTGAAATCAGCAACGACGTGGAGGTGGTGAAGGATCAGTT | SNDVEVVKDQFHTHE |
| | | | CCACACACACGAGGGCTACATGATGGACCTGACTGCCCATCAGGGAC | GYMMDLTAHQGRVG |
| | | | GCGTGGGCAACATTCTTCAGCTGGGCTCCAAGCTGATCGGCACCGGC | NILQLGSKLIGTGKLSE |
| | | | AAGCTGTCCGAGGACGAAGAGACCGAAGTGCAGGAACAGATGAATT | DEETEVQEQMNLLNSR |
| | | | TGCTGAACTCCCGCTGGGAGTGCCTCAGAGTCGCATCAATGGAGAAA | WECLRVASMEKQSNL |
| | | | CAGTCCAACCTGCACAGGGTGTTGATGGATCTCCAGAATCAGAAGTT | HRVLMDLQNQKLKEL |
| | | | GAAGGAACTGAACGACTGGCTCACCAAGACCGAAGAGAGAACCCGG | NDWLTKTEERTRKME |
| | | | AAGATGGAAGAGGAGCCACTGGGCCCTGATCTTGAGGATCTGAAGCG | EEPLGPDLEDLKRQVQ |
| | | | GCAGGTCCAACAGCACAAGGTGCTCCAAGAGGACCTCGAGCAGGAG | QHKVLQEDLEQEQVR |
| | | | CAGGTCCGCGTCAACTCCCTGACCCACATGGTGGTGGTGGTGGATGA | VNSLTHMVVVVDESS |
| | | | ATCAAGCGGCGACCATGCAACCGCAGCCTTGGAGGAACAGCTCAAGG | GDHATAALEEQLKVL |
| | | | TCCTGGGTGACCGCTGGGCCAACATCTGCCGGTGGACAGAGGACCGG | GDRWANICRWTEDRW |
| | | | TGGGTGCTGCTTCAGGACATCCTCCTGAAGTGGCAACGCCTGACCGA | VLLQDILLKWQRLTEE |
| | | | GGAGCAATGCTTGTTCAGCGCCTGGCTCTCTGAGAAGGAGGATGCAG | QCLFSAWLSEKEDAVN |
| | | | TCAACAAAATCCACACCACCGGCTTCAAGGATCAGAACGAGATGCTG | KIHTTGFKDQNEMLSS |
| | | | AGCTCCCTGCAGAAGCTCGCCGTCCTGAAGGCTGACCTCGAGAAGAA | LQKLAVLKADLEKKK |
| | | | GAAGCAGTCCATGGGTAAGCTGTACTCCCTGAAGCAGGACCTGCTCT | QSMGKLYSLKQDLLST |
| | | | CTACTCTGAAGAATAAGTCCGTCACCCAGAAGACCGAAGCCTGGCTC | LKNKSVTQKTEAWLD |
| | | | GATAACTTCGCCCGGTGCTGGGACAACCTCGTTCAGAAACTCGAGAA | NFARCWDNLVQKLEK |
| | | | GAGCACCGCTCAAATCTCCCAGGCCGTCACAACCACCCAGCCCTCTCT | STAQISQAVTTTQPSLT |
| | | | GACCCAAACCACAGTCATGGAGACCGTGACCACCGTTACCACCCGGG | QTTVMETVTTVTTREQ |
| | | | AGCAGATCCTCGTCAAGCACGCCCAAGAGGAACTTCCTCCTCCTCCTC | ILVKHAQEELPPPPPQK |
| | | | CACAGAAGAAAAGGCAAATCACCGTCGACTCCGAAATCCGCAAGCG | KRQITVDSEIRKRLDVD |
| | | | CCTCGACGTGGACATCACCGAGCTCCATAGCTGGATTACCCGCAGCG | ITELHSWITRSEAVLQS |
| | | | AGGCAGTTCTGCAGTCCCCTGAGTTCGCCATCTTCCGCAAGGAGGGTA | PEFAIFRKEGNFSDLKE |
| | | | ATTTCTCCGACCTTAAAGAGAAGGTCAACGCCATCGAGCGCGAGAAG | KVNAIEREKAEKFRKL |
| | | | GCCGAGAAGTTCCGCAAGCTTCAGGACGCCTCACGGTCAGCACAGGC | QDASRSAQALVEQMV |
| | | | ATTGGTGGAGCAGATGGTCAACGAGGGCGTCAACGCCGATAGCATCA | NEGVNADSIKQASEQL |
| | | | AGCAGGCTTCCGAACAGCTGAACAGCCGGTGGATCGAGTTCTGCCAG | NSRWIEFCQLLSERLN |
| | | | CTCCTCAGCGAACGGCTCAACTGGCTCGAGTATCAGAACAACATCAT | WLEYQNNIIAFYNQLQ |
| | | | CGCCTTCTATAACCAGCTGCAACAACTTGAGCAAATGACCACCACCG | QLEQMTTTAENWLKIQ |
| | | | CCGAGAACTGGCTGAAGATCCAGCCCACCACCCCTTCCGAACCCACC | PTTPSEPTAIKSQLKICK |
| | | | GCCATCAAGTCTCAGCTGAAGATTTGCAAGGACGAGGTCAATCGGCT | DEVNRLSDLQPQIERL |
| | | | GAGTGACCTCCAACCCCAGATTGAGCGGCTCAAGATCCAAAGTATTG | KIQSIALKEKGQGPMFL |
| | | | CCCTGAAGGAGAAGGGCCAAGGCCCCATGTTCCTCGACGCCGACTTC | DADFVAFTNHFKQVFS |
| | | | GTCGCCTTCACCAACCACTTCAAGCAAGTCTTCAGTGACGTGCAGGCC | DVQAREKELQTIFDTLP |
| | | | CGCGAAAAGGAGCTTCAGACAATCTTCGATACCCTTCCTCCTATGCGG | PMRYQETMSAIRTWV |
| | | | TACCAGGAGACCATGTCAGCAATCAGGACCTGGGTGCAGCAATCAGA | QQSETKLSIPQLSVTDY |
| | | | GACCAAGCTGTCAATCCCACAGCTCAGCGTTACCGACTACGAGATCA | EIMEQRLGELQALQSS |
| | | | TGGAGCAAAGGTTGGGAGAGCTGCAAGCCCTGCAGTCCTCCCTGCAG | LQEQQSGLYYLSTTVK |
| | | | GAGCAGCAGTCCGGGCTCTACTACCTGAGCACCACCGTTAAAGAGAT | EMSKKAPSEISRKYQSE |
| | | | GTCCAAGAAGGCCCCTAGCGAAATCAGTAGAAAGTACCAGTCAGAGT | FEEIEGRWKKLSSQLV |
| | | | TCGAAGAGATTGAGGGGCGGTGGAAGAAGCTCAGCTCCCAACTTGTG | EHCQKLEEQMNKLRKI |
| | | | GAGCACTGCCAGAAGCTCGAAGAGCAAATGAACAAGCTCCGGAAGA | QNHIQTLKKWMAEVD |
| | | | TTCAGAACCACATTCAAACCTTGAAGAAGTGGATGGCAGAGGTCGAC | VFLKEEWPALGDSEIL |
| | | | GTTTTCCTCAAGGAGGAGTGGCCCGCACTTGGGGACTCCGAGATTCTT | KKQLKQCRLLVSDIQTI |
| | | | AAGAAGCAACTGAAGCAATGCCGGCTGCTGGTGTCTGACATCCAAAC | QPSLNSVNEGGQKIKN |
| | | | CATCCAGCCCTCCCTGAACTCTGTGAACGAAGGAGGTCAGAAGATCA | EAEPEFASRLETELKEL |
| | | | AGAACGAGGCTGAGCCCGAGTTCGCCTCACGGCTGGAGACCGAACTC | NTQWDHMCQQVYAR |
| | | | AAAGAGCTGAATACCCAGTGGGACCACATGTGCCAGCAGGTGTACGC | KEALKGGLEKTVSLQK |
| | | | TCGGAAGGAAGCTCTGAAGGGAGGTCTGGAGAAGACCGTCTCTCTGC | DLSEMHEWMTQAEEE |
| | | | AAAAGGACTTGAGCGAGATGCACGAGTGGATGACCCAAGCTGAGGA | YLERDFEYKTPDELQK |
| | | | GGAGTACCTGGAGCGGGATTTCGAGTACAAGACACCCGACGAGCTGC | AVEEMKRAKEEAQQK |
| | | | AGAAAGCCGTCGAAGAGATGAAGCGGGCAAAGGAGGAGGCACAGCA | EAKVKLLTESVNSVIA |
| | | | GAAGGAGGCTAAAGTCAAGTTGCTGACCGAGTCCGTGAACTCCGTGA | QAPPVAQEALKKELET |
| | | | TCGCTCAGGCCCCACCAGTTGCCCAAGAAGCATTGAAGAAGGAGTTG | LTTNYQWLCTRLNGK |
| | | | GAAACCCTCACCACCAACTACCAGTGGCTCTGCACCAGGCTGAACGG | CKTLEEVWACWHELL |
| | | | GAAGTGCAAGACCCTTGAGGAGGTGTGGGCTTGCTGGCATGAGCTGC | SYLEKANKWLNEVEF |
| | | | TCTCCTATCTCGAGAAGGCCAACAAATGGCTGAATGAGGTGGAGTTC | KLKTTENIPGGAEEISE |
| | | | AAGCTGAAGACCACCGAGAATATCCCTGGTGGGGCAGAGGAGATCTC | VLDSLENLMRHSEDNP |
| | | | CGAGGTTCTCGACTCACTTGAGAATCTGATGCGGCATAGCGAGGACA | NQIRILAQTLTDGGVM |
| | | | ACCCCAACCAGATCAGAATCTTGGCTCAGACCCTTACCGACGGAGGG | DELINEELETFNSRWRE |
| | | | GTGATGGATGAGCTGATCAACGAAGAGCTCGAGACCTTCAACAGCAG | LHEEAVRRQKLLEQSI |
| | | | GTGGCGCGAGTTGCACGAGGAGGCTGTCAGGCGGCAGAAGCTCCTCG | QSAQETEKSLHLIQESL |
| | | | AGCAAAGCATCCAGTCTGCCCAGGAGACAGAGAAAAGTCTGCACCTG | TFIDKQLAAYIADKVD |
| | | | ATCCAGGAATCCCTGACCTTCATCGATAAGCAGCTGGCTGCTTACATC | AAQMPQEAQKIQSDLT |
| | | | GCCGACAAGGTGGACGCTGCTCAGATGCCACAAGAGGCTCAGAAGAT | SHEISLEEMKKHNQGK |
| | | | CCAGTCCGACCTCACCTCCCATGAGATCTCCCTCGAGGAGATGAAGA | EAAQRVLSQIDVAQKK |
| | | | AGCACAACCAGGGTAAGGAGGCTGCCCAGAGGGTCCTGTCACAGATC | LQDVSMKFRLFQKPAN |
| | | | GACGTTGCACAGAAGAAGCTCCAGGACGTCAGCATGAAGTTCCGGCT | FEQRLQESKMILDEVK |
| | | | GTTCCAAAAGCCCGCAAATTTTGAGCAGAGACTCCAGGAGTCCAAGA | MHLPALETKSVEQEVV |
| | | | TGATCCTCGACGAGGTGAAGATGCACCTGCCTGCCCTGGAGACCAAG | QSQLNHCVNLYKSLSE |
| | | | TCTGTGGAGCAGGAGGTCGTGCAATCCCAGTTGAACCACTGCGTGAA | VKSEVEMVIKTGRQIV |
| | | | CCTGTACAAGAGCCTGTCCGAGGTCAAAAGCGAGGTCGAGATGGTGA | QKKQTENPKELDERVT |
| | | | TCAAAACCGGCAGGCAAATCGTGCAAAAGAAGCAAACCGAGAACCC | ALKLHYNELGAKVTER |
| | | | CAAGGAACTGGATGAGCGGGTGACCGCCCTTAAGCTCCACTACAACG | KQQLEKCLKLSRKMR |
| | | | AGCTCGGTGCCAAAGTCACCGAACGGAAGCAGCAGCTCGAGAAATG | KEMNVLTEWLAATDM |
| | | | CCTCAAGCTGAGCCGCAAGATGCGGAAGGAGATGAACGTCCTGACTG | ELTKRSAVEGMPSNLD |
| | | | AGTGGCTGGCTGCCACCGACATGGAGCTGACCAAGAGGAGCGCTGTG | SEVAWGKATQKEIEKQ |
| | | | GAGGGAATGCCCTCCAACCTGGATAGCGAGGTCGCCTGGGGTAAGGC | KVHLKSITEVGEALKT |
| | | | CACCCAGAAGGAAATTGAGAAGCAGAAGGTCCACCTGAAGTCCATCA | VLGKKETLVEDKLSLL |
| | | | CCGAGGTTGGGGAAGCCCTGAAGACCGTCCTGGGCAAGAAGGAGAC | NSNWIAVTSRAEEWLN |
| | | | CCTGGTGGAAGACAAGCTGTCACTCCTGAACTCCAACTGGATCGCAG | LLLEYQKHMETFDQN |
| | | | TGACCAGTCGCGCCGAAGAGTGGCTCAACCTCCTCCTGGAGTACCAG | VDHITKWIIQADTLLDE |
| | | | AAGCACATGGAGACCTTCGACCAGAACGTCGACCACATCACCAAGTG | SEKKKPQQKEDVLKRL |
| | | | GATCATCCAGGCAGACACCCTTCTCGACGAGTCTGAAAAGAAGAAGC | KAELNDIRPKVDSTRD |
| | | | CCCAGCAAAAGGAGGACGTCCTGAAGAGACTCAAGGCTGAGCTCAA | QAANLMANRGDHCRK |
| | | | CGACATCCGCCCAAAGGTGGACTCCACTCGGGACCAGGCAGCTAACT | LVEPQISELNHRFAAIS |
| | | | TGATGGCCAATAGGGGCGATCACTGCCGGAAGCTGGTGGAGCCCCAG | HRIKTGKASIPLKELEQ |
| | | | ATCAGCGAGCTCAACCACAGGTTCGCCGCCATCAGTCACCGGATCAA | FNSDIQKLLEPLEAEIQ |
| | | | GACTGGGAAGGCCTCCATCCCACTTAAGGAGCTGGAACAGTTCAACT | QGVNLKEEDFNKDMN |
| | | | CCGACATTCAGAAGCTGCTCGAACCTCTGGAGGCCGAGATCCAGCAA | EDNEGTVKELLQRGDN |
| | | | GGGGTCAACCTGAAGGAGGAGGACTTCAACAAGGATATGAACGAGG | LQQRITDERKREEIKIK |
| | | | ATAACGAGGGAACCGTTAAGGAGCTTCTCCAGCGCGGCGACAACCTG | QQLLQTKHNALKDLRS |
| | | | CAACAGCGGATCACCGACGAGCGGAAACGGGAGGAGATCAAGATCA | QRRKKALEISHQWYQ |
| | | | AGCAACAGCTGCTGCAGACCAAGCATAACGCCCTGAAAGACCTGCGG | YKRQADDLLKCLDDIE |
| | | | TCTCAACGGCGGAAGAAGGCCCTGGAAATCTCCCATCAGTGGTACCA | KKLASLPEPRDERKIKE |
| | | | GTACAAGCGGCAGGCCGATGACCTGCTCAAGTGCCTGGACGACATTG | IDRELQKKKEELNAVR |
| | | | AGAAGAAGCTTGCCAGTCTGCCTGAGCCCCGCGATGAGAGAAAGATC | RQAEGLSEDGAAMAV |
| | | | AAGGAGATTGACCGGGAGCTTCAAAAGAAGAAGGAGGAGCTCAATG | EPTQIQLSKRWREIESK |
| | | | CTGTCCGGAGGCAAGCTGAGGGTCTGTCCGAAGACGGTGCCGCTATG | FAQFRRLNFAQIHTVR |
| | | | GCTGTGGAGCCCACCCAGATCCAGCTGAGTAAGCGGTGGCGCGAAAT | EETMMVMTEDMPLEIS |
| | | | CGAGTCAAAGTTCGCACAGTTCCGGCGGCTGAACTTCGCCCAGATCC | YVPSTYLTEITHVSQAL |
| | | | ATACCGTCCGGGAGGAAACCATGATGGTCATGACCGAGGACATGCCT | LEVEQLLNAPDLCAKD |
| | | | CTGGAGATTTCTTACGTCCCTTCCACATACCTGACTGAAATCACCCAC | FEDLFKQEESLKNIKDS |
| | | | GTGTCCCAAGCCCTTCTGGAAGTGGAGCAGCTCCTGAATGCCCCTGAC | LQQSSGRIDIIHSKKTA |
| | | | TTGTGCGCCAAGGATTTCGAGGACCTCTTCAAGCAGGAGGAGAGCCT | ALQSATPVERVKLQEA |
| | | | GAAGAATATCAAGGACTCTCTCCAACAGTCCTCCGGTCGGATCGACA | LSQLDFQWEKVNKMY |
| | | | TCATCCACTCCAAGAAGACTGCAGCCCTCCAGTCAGCAACCCCTGTCG | KDRQGRFDRSVEKWR |
| | | | AGCGCGTGAAGCTCCAAGAAGCCCTCTCTCAGTTGGACTTCCAATGG | RFHYDIKIFNQWLTEA |
| | | | GAGAAAGTCAACAAGATGTACAAGGACCGGCAAGGGCGCTTCGACC | EQFLRKTQIPENWEHA |
| | | | GGTCTGTCGAGAAGTGGCGGAGGTTCCACTACGACATCAAGATCTTC | KYKWYLKELQDGIGQ |
| | | | AACCAGTGGCTGACCGAGGCTGAACAGTTCCTGCGGAAAACCCAGAT | RQTVVRTLNATGEEIIQ |
| | | | CCCTGAGAACTGGGAGCACGCCAAGTACAAGTGGTACCTCAAAGAGT | QSSKTDASILQEKLGSL |
| | | | TGCAGGATGGGATCGGCCAAAGGCAGACCGTGGTCCGGACCCTCAAC | NLRWQEVCKQLSDRK |
| | | | GCCACCGGTGAGGAGATCATCCAGCAGTCATCCAAGACCGATGCATC | KRLEEQKNILSEFQRDL |
| | | | CATCCTGCAGGAGAAGCTCGGGTCTCTGAACCTGCGGTGGCAGGAGG | NEFVLWLEEADNIASIP |
| | | | TCTGTAAGCAGCTCTCCGACCGGAAGAAGCGGCTGGAAGAGCAAAA | LEPGKEQQLKEKLEQV |
| | | | GAACATCCTGAGCGAGTTCCAGCGCGATCTGAACGAGTTCGTGCTCT | KLLVEELPLRQGILKQL |
| | | | GGTTGGAGGAGGCTGATAACATCGCCAGCATTCCCCTGGAGCCCGGT | NETGGPVLVSAPISPEE |
| | | | AAGGAACAACAGCTTAAGGAAAAGCTGGAGCAGGTTAAGCTCCTGGT | QDKLENKLKQTNLQWI |
| | | | GGAGGAGCTGCCACTCCGGCAAGGAATCCTCAAGCAGCTTAACGAGA | KVSRALPEKQGEIEAQI |
| | | | CTGGTGGACCCGTGCTCGTCTCCGCTCCCATCAGCCCAGAAGAGCAG | KDLGQLEKKLEDLEEQ |
| | | | GACAAGCTCGAAAACAAGCTGAAGCAGACCAATCTGCAATGGATCA | LNHLLLWLSPIRNQLEI |
| | | | AGGTGTCCCGGGCTCTCCCTGAGAAGCAGGGAGAAATCGAGGCACAG | YNQPNQEGPFDVKETE |
| | | | ATCAAGGACCTGGGCCAGCTTGAGAAGAAACTCGAAGACCTCGAGG | IAVQAKQPDVEEILSKG |
| | | | AGCAACTTAATCACCTGCTGCTGTGGCTCTCTCCCATCCGGAATCAGC | QHLYKEKPATQPVKRK |
| | | | TGGAAATCTACAACCAACCAAACCAGGAGGGCCCCTTCGACGTGAAA | LEDLSSEWKAVNRLLQ |
| | | | GAAACCGAGATCGCCGTGCAGGCTAAGCAGCCCGACGTCGAGGAGA | ELRAKQPDLAPGLTTIG |
| | | | TCCTGTCCAAGGGGCAGCACCTGTATAAGGAGAAGCCTGCTACACAG | ASPTQTVTLVTQPVVT |
| | | | CCCGTGAAGCGGAAGCTTGAAGACCTCAGCAGCGAGTGGAAGGCCGT | KETAISKLEMPSSLMLE |
| | | | CAACCGGCTCTTGCAAGAACTTCGGGCCAAGCAACCAGACCTGGCTC | VPALADFNRAWTELTD |
| | | | CAGGCCTGACAACCATCGGCGCTTCTCCAACCCAAACCGTCACCCTG | WLSLLDQVIKSQRVMV |
| | | | GTGACCCAGCCAGTCGTCACTAAAGAAACCGCAATCTCTAAGCTCGA | GDLEDINEMIIKQKAT |
| | | | GATGCCCTCCTCCCTGATGCTGGAAGTGCCCGCTCTGGCTGACTTCAA | MQDLEQRRPQLEELIT |
| | | | CCGCGCATGGACCGAGCTTACCGATTGGCTGAGCCTGCTCGACCAGG | AAQNLKNKTSNQEAR |
| | | | TCATTAAGTCCCAGCGCGTGATGGTGGGTGATCTGGAGGACATTAAC | TIITDRIERIQNQWDEV |
| | | | GAGATGATCATTAAGCAGAAGGCCACCATGCAGGATCTGGAGCAACG | QEHLQNRRQQLNEML |
| | | | GCGGCCACAACTGGAGGAACTGATCACCGCTGCCCAGAATCTCAAGA | KDSTQWLEAKEEAEQ |
| | | | ACAAGACCTCTAACCAGGAGGCTCGCACCATCATCACCGATAGGATT | VLGQARAKLESWKEG |
| | | | GAGCGGATCCAAAACCAGTGGGACGAGGTCCAAGAGCATCTCCAAA | PYTVDAIQKKITETKQL |
| | | | ACCGGCGGCAGCAACTGAATGAAATGCTCAAGGACAGCACCCAATG | AKDLRQWQTNVDVAN |
| | | | GCTGGAGGCCAAGGAAGAAGCAGAACAAGTGCTTGGGCAGGCAAGG | DLALKLLRDYSADDTR |
| | | | GCTAAGCTGGAATCCTGGAAGGAGGGGCCATACACCGTGGACGCAAT | KVHMITENINASWRSI |
| | | | CCAGAAGAAGATCACCGAGACCAAGCAGCTTGCCAAGGATCTGCGCC | HKRVSEREAALEETHR |
| | | | AGTGGCAGACCAACGTGGACGTCGCAAACGACCTGGCACTCAAGCTG | LLQQFPLDLEKFLAWL |
| | | | CTCCGGGATTACTCCGCCGACGATACCCGGAAGGTGCACATGATCAC | TEAETTANVLQDATRK |
| | | | CGAAAACATCAACGCCTCCTGGCGCTCTATCCACAAGCGGGTGTCTG | ERLLEDSKGVKELMKQ |
| | | | AGAGGGAGGCTGCTCTCGAGGAGACCCACAGACTCCTGCAGCAGTTC | WQDLQGEIEAHTDVY |
| | | | CCTCTCGACCTTGAGAAGTTCCTGGCCTGGCTCACAGAGGCTGAGACC | HNLDENSQKILRSLEGS |
| | | | ACCGCCAACGTCCTCCAGGACGCTACCAGGAAGGAGCGCCTCTTGGA | DDAVLLQRRLDNMNF |
| | | | GGACAGCAAAGGCGTCAAGGAACTCATGAAGCAGTGGCAGGATCTC | KWSELRKKSLNIRSHL |
| | | | CAGGGCGAGATTGAGGCCCATACCGACGTGTACCACAACCTCGACGA | EASSDQWKRLHLSLQE |
| | | | GAACTCCCAGAAGATCCTGAGATCCCTCGAGGGTAGCGACGATGCAG | LLVWLQLKDDELSRQ |
| | | | TCCTGCTGCAACGCAGGCTGGATAACATGAACTTCAAATGGTCCGAG | APIGGDFPAVQKQNDV |
| | | | CTGCGCAAGAAGTCCCTCAATATCCGCAGTCATCTCGAGGCTAGCTCC | HRAFKRELKTKEPVIM |
| | | | GACCAGTGGAAGCGGCTGCACCTCTCCCTCCAAGAGCTCCTTGTCTGG | STLETVRIFLTEQPLEG |
| | | | CTGCAGTTGAAGGATGACGAACTGTCACGGCAGGCTCCCATCGGTGG | LEKLYQEPRELPPEERA |
| | | | CGATTTTCCAGCTGTGCAGAAACAGAACGACGTGCACCGGGCCTTCA | QNVTRLLRKQAEEVNT |
| | | | AGCGCGAACTCAAGACCAAGGAGCCCGTGATCATGTCCACCCTCGAG | EWEKLNLHSADWQRK |
| | | | ACCGTGCGGATCTTCCTGACAGAGCAGCCCTTGGAGGGCCTCGAGAA | IDETLERLRELQEATDE |
| | | | GCTGTACCAGGAACCCAGAGAGCTTCCACCCGAAGAAAGGGCACAA | LDLKLRQAEVIKGSWQ |
| | | | AATGTGACCCGGCTTCTGCGGAAGCAGGCCGAAGAGGTCAACACCGA | PVGDLLIDSLQDHLEK |
| | | | GTGGGAGAAGCTGAACCTGCACTCCGCCGATTGGCAGCGGAAAATCG | VKALRGELAPLKENVS |
| | | | ACGAGACACTGGAGCGGCTGCAAGAACTGCAGGAAGCCACAGACGA | HVNDLARQLTTLGIQL |
| | | | GCTCGACCTCAAGCTGCGCCAGGCAGAGGTTATCAAGGGCTCTTGGC | SPYNLSTLEDLNTRWK |
| | | | AGCCCGTCGGAGACTTGCTGATCGACAGCCTCCAGGACCACCTGGAG | LLQVAVEDRVRQLHE |
| | | | AAGGTGAAGGCTCTGCGCGGAGAAATCGCCCCTCTGAAAGAGAACGT | AHRDFGPASQHELSTS |
| | | | CAGTCACGTGAACGACCTCGCTCGGCAGCTCACCACCCTGGGGATCC | VQGPWERAISPNKVPY |
| | | | AGCTCAGCCCCTACAACCTGAGTACCCTGGAGGACCTGAATACCCGC | YINHETQTTCWDHPK |
| | | | TGGAAACTGCTCCAGGTGGCAGTGGAGGATCGGGTCCGGCAGCTTCA | MTELYQSLADLNNVRF |
| | | | CGAGGCTCATCGGGACTTCGGCCCAGCTAGCCAGCACTTCCTGTCCAC | SAYRTAMKLRRLQKA |
| | | | CTCCGTTCAAGGGCCCTGGGAGAGGGCAATCTCTCCTAACAAGGTCC | LCLDLLSLSAACDALD |
| | | | CCTACTACATCAACCACGAGACTCAGACCACCTGCTGGGACCATCCC | QHNLKQNDQPMDILQI |
| | | | AAGATGACCGAGCTCTACCAGTCTCTGGCCGACCTGAACAACGTGCG | INCLTTIYDRLEQEHNN |
| | | | CTTCAGCGCTTACCGGACTGCAATGAAGCTGCGCCGCCTGCAGAAGG | LVNVPLCVDMCLNWL |
| | | | CACTCTGCCTCGACCTCCTGTCACTCAGCGCAGCTTGCGACGCCCTTG | LNVYDTGRTGRIRVLS |
| | | | ACCAGCATAACTTGAAGCAAAACGACCAGCCCATGGATATCCTGCAG | FKTGIISLCKAHLEDKY |
| | | | ATCATCAACTGCCTCACCACAATCTACGACCGGCTTGAGCAAGAGCA | RYLFKQVASSTGFCDQ |
| | | | CAATAATCTGGTGAACGTTCCCCTCTGCGTGGACATGTGCTTGAACTG | RRLGLLLHDSIQIPRQL |
| | | | GCTGCTGAACGTGTACGACACCGGTCGCACAGGAAGAATCCGGGTGC | GEVASFGGSNIEPSVRS |
| | | | TGTCATTCAAGACCGGAATCATCTCCCTGTGCAAGGCCCACCTGGAG | CFQFANNKPEIEAALFL |
| | | | GACAAGTACCGGTACCTGTTCAAGCAGGTGGCTAGCTCCACCGGGTT | DWMRLEPQSMVWLPV |
| | | | CTGCGACCAGAGAAGGTTGGGGCTGCTGCTGCACGATAGCATCCAGA | LHRVAAAETAKHQAK |
| | | | TTCCCAGGCAGCTCGGCGAAGTCGCCTCCTTCGGAGGCTCCAACATCG | CNICKECPIIGFRYRSLK |
| | | | AGCCATCCGTCCGGAGCTGCTTTCAGTTCGCTAACAACAAGCCAGAG | HFNYDICQSCFFSGRV |
| | | | ATCGAGGCAGCCCTGTTCCTGGACTGGATGCGGCTGGAACCACAGTC | AKGHKMHYPMVEYCT |
| | | | CATGGTCTGGCTCCCCGTGCTGCACAGAGTCGCCGCTGCCGAAACAG | PTTSGEDVRDFAKVLK |
| | | | CCAAGCACCAGGCCAAGTGCAATATCTGTAAGGAGTGCCCCATCATC | NKFRTKRYFAKHPRM |
| | | | GGCTTCCGGTACCGGTCCCTCAAGCACTTCAACTACGATATCTGCCAG | GYLPVQTVLEGDNME |
| | | | TCCTGCTTCTTCAGTGGCCGGGTGGCCAAAGGTCACAAGATGCACTAC | TPVTLINFWPVDSAPAS |
| | | | CCCATGGTGGAGTACTGTACACCCACTACCTCCGGCGAGGATGTGAG | SPQLSHDDTHSRIEHYA |
| | | | AGACTTCGCAAAAGTCTTGAAGAACAAGTTCCGGACCAAGCGGTATT | SRLAEMENSNGSYLND |
| | | | TCGCCAAGCATCCACGGATGGGCTACCTCCCCGTCCAGACCGTGCTCG | SISPNESIDDEHLLIQHY |
| | | | AGGGCGACAACATGGAAACCCCAGTCACCTTGATCAACTTCTGGCCC | CQSLNQDSPLSQPRSPA |
| | | | GTGGATAGCGCTCCAGCTAGCTCCCCTCAGCTGTCTCACGACGACACC | QILISLESEERGELERIL |
| | | | CACAGCAGGATCGAGCACTACGCATCACGGCTGGCAGAGATGGAGA | ADLEEENRNLQAEYDR |
| | | | ACTCCAACGGGTCCTACCTGAACGACAGTATCAGCCCCAACGAATCC | LKQQHEHKGLSPLPSPP |
| | | | ATCGACGACGAGCACCTGCTCATCCAACACTACTGCCAGTCCCTCAAC | EMMPTSPQSPRDAELI |
| | | | CAGGACTCTCCTCTGTCTCAGCCCCGGTCACCAGCACAAATCCTGATC | AEAKLLRQHKGRLEAR |
| | | | TCTCTGGAGAGTGAGGAGAGAGGCGAGCTCGAGCGCATCCTGGCCGA | MQILEDHNKQLESQLH |
| | | | TCTTGAAGAAGAGAACCGGAACCTCCAGGCCGAGTACGACAGGCTCA | RLRQLLEQPQAEAKVN |
| | | | AACAGCAGCACGAGCACAAGGGGCTCAGTCCCTTGCCCTCACCTCCA | GTTVSSPSTSLQRSDSS |
| | | | GAGATGATGCCCACCTCTCCACAGTCACCCCGGGACGCTGAACTGAT | QPMLLRVVGSQTSDSM |
| | | | CGCAGAGGCTAAGCTGCTGCGGCAACACAAGGGACGGCTGGAAGCT | GEEDLLSPPQDTSTGLE |
| | | | CGGATGCAGATTCTGGAGGACCACAACAAGCAGCTCGAATCCCAGCT | EVMEQLNNSFPSSRGR |
| | | | CCATCGCTTGCGCCAGCTCCTGGAGCAACCCCAAGCCGAGGCCAAGG | NTPGKPMREDTM (SEQ |
| | | | TCAACGGGACCACCGTGTCCTCACCCAGTACATCCCTGCAGCGGTCCG | ID NO: 10) |
| | | | ACAGCAGCCAGCCTATGCTCCTCCGGGTTGTGGGGTCCCAGACCTCCG | |
| | | | ATTCCATGGGTGAGGAGGACCTTCTCTCACCACCACAGGACACCTCA | |
| | | | ACCGGCCTCGAGGAGGTGATGGAGCAACTGAACAACTCTTTTCCTTCC | |
| | | | TCACGCGGCCGGAATACCCCAGGCAAGCCTATGCGCGAGGACACAAT | |
| | | | Gtaa (SEQ ID NO: 9) | |
| Full | Wild Type | NP_003997 | ATGCTGTGGTGGGAGGAGGTTGAGGATTGCTACGAAAGGGAGGATGT | MLWWEEVEDCYERED |
| Length | protein | | CCAAAAGAAGACCTTCACCAAATGGGTGAACGCCCAGTTCAGCAAGT | VQKKTFTKWVNAQFS |
| human | (CO | | TCGGTAAGCAGCACATTGAGAACCTCTTTAGCGACCTCCAGGACGGA | KFGKQHIENLFSDLQD |
| dystrophin | nucleotide | | CGCCGCTTGCTGGATTTGCTGGAGGGTCTGACCGGGCAGAAGCTGCC | GRRLLDLLEGLTGQKL |
| | seq-2B) | | CAAGGAAAAGGGCTCAACCAGGGTCCACGCCCTCAACAACGTGAAC | PKEKGSTRVHALNNVN |
| | | | AAGGCCCTGAGGGTGCTGCAGAACAATAACGTCGACCTGGTTAACAT | KALRVLQNNNVDLVNI |
| | | | CGGCTCCACCGACATCGTCGATGGCAATCACAAGCTCACCCTCGGAC | GSTDIVDGNHKLTLGLI |
| | | | TGATTTGGAACATCATCCTCCACTGGCAGGTGAAGAACGTCATGAAG | WNIILHWQVKNVMKN |
| | | | AACATCATGGCCGGCCTGCAACAGACCAACTCCGAGAAGATCTTGCT | IMAGLQQTNSEKILLS |
| | | | GAGCTGGGTCCGGCAGTCCACTCGGAACTATCCCCAGGTCAACGTCA | WVRQSTRNYPQVNVIN |
| | | | TCAACTTCACCACCAGCTGGTCCGACGGACTCGCCCTGAACGCCCTCA | FTTSWSDGLALNALIH |
| | | | TCCACTCTCACCGGCCAGACCTGTTCGACTGGAACAGCGTCGTGTGCC | SHRPDLFDWNSVVCQ |
| | | | AGCAAAGCGCAACCCAGAGGCTCGAACACGCCTTCAACATCGCCAGG | QSATQRLEHAFNIARY |
| | | | TACCAGCTCGGGATCGAGAAGCTCCTCGACCCCGAAGACGTCGACAC | QLGIEKLLDPEDVDTT |
| | | | CACCTACCCCGACAAGAAGTCCATCCTTATGTACATCACTAGCCTGTT | YPDKKSILMYITSLFQV |
| | | | CCAGGTTCTGCCCCAACAAGTGAGCATCGAGGCTATCCAGGAAGTCG | LPQQVSIEAIQEVEMLP |
| | | | AGATGTTGCCCAGACCACCAAAGGTGACAAAAGAGGAGCACTTCCAA | RPPKVTKEEHFQLHHQ |
| | | | CTGCATCACCAGATGCACTACTCCCAGCAGATCACAGTGAGCCTGGC | MHYSQQITVSLAQGYE |
| | | | TCAGGGTTACGAGAGGACCTCAAGCCCAAAGCCCCGGTTCAAGTCCT | RTSSPKPRFKSYAYTQ |
| | | | ACGCATACACCCAGGCCGCTTACGTCACCACCTCCGATCCCACCAGGT | AAYVTTSDPTRSPFPSQ |
| | | | CACCATTCCCATCCCAGCATCTCGAGGCACCCGAGGATAAGTCTTTCG | HLEAPEDKSFGSSLME |
| | | | GCTCCAGTCTCATGGAGTCCGAGGTCAATCTGGACCGGTACCAAACA | SEVNLDRYQTALEEVL |
| | | | GCACTCGAGGAAGTGCTCTCTTGGCTCCTGTCCGCAGAAGATACCCTC | SWLLSAEDTLQAQGEI |
| | | | CAGGCTCAGGGTGAGATTTCCAACGACGTCGAGGTGGTGAAGGACCA | SNDVEVVKDQFHTHE |
| | | | GTTCCACACCCACGAGGGCTACATGATGGACCTCACAGCTCACCAGG | GYMMDLTAHQGRVG |
| | | | GGAGAGTGGGTAACATTCTGCAACTTGGCTCCAAACTCATCGGGACC | NILQLGSKLIGTGKLSE |
| | | | GGCAAGCTGAGCGAGGATGAAGAGACCGAGGTCCAGGAGCAGATGA | DEETEVQEQMNLLNSR |
| | | | ATTTGCTGAACTCCCGGTGGGAGTGTCTGCGGGTCGCTAGCATGGAG | WECLRVASMEKQSNL |
| | | | AAGCAGTCTAACCTTCACCGGGTCCTCATGGACCTGCAGAACCAGAA | HRVLMDLQNQKLKEL |
| | | | ACTCAAAGAGCTCAACGACTGGCTGACCAAGACCGAGGAGAGGACA | NDWLTKTEERTRKME |
| | | | CGCAAGATGGAGGAAGAACCACTGGGCCCCGATCTTGAGGACTTGAA | EEPLGPDLEDLKRQVQ |
| | | | GCGGCAGGTGCAACAGCATAAAGTGCTCCAGGAGGACCTGGAGCAA | QHKVLQEDLEQEQVR |
| | | | GAGCAGGTGCGGGTGAACTCACTCACCCACATGGTCGTCGTCGTGGA | VNSLTHMVVVVDESS |
| | | | CGAGTCTTCCGGCGACCATGCTACCGCTGCCCTGGAGGAACAGCTGA | GDHATAALEEQLKVL |
| | | | AGGTGCTTGGGGACAGATGGGCAAACATCTGCAGGTGGACCGAGGAT | GDRWANICRWTEDRW |
| | | | CGGTGGGTTCTCTTGCAGGACATTCTGCTGAAGTGGCAGCGCCTGACC | VLLQDILLKWQRLTEE |
| | | | GAAGAGCAATGCCTCTTCAGTGCCTGGCTGTCCGAAAAGGAGGACGC | QCLFSAWLSEKEDAVN |
| | | | TGTGAACAAGATCCATACCACCGGCTTCAAGGATCAGAATGAGATGC | KIHTTGFKDQNEMLSS |
| | | | TGTCATCTCTGCAAAAGCTGGCCGTCCTCAAGGCCGATCTCGAGAAG | LQKLAVLKADLEKKK |
| | | | AAGAAGCAATCCATGGGCAAGCTGTACAGCCTGAAGCAGGATCTGCT | QSMGKLYSLKQDLLST |
| | | | GTCCACACTCAAGAACAAGAGCGTTACCCAAAAGACCGAGGCCTGGC | LKNKSVTQKTEAWLD |
| | | | TCGACAACTTCGCTCGGTGCTGGGACAACCTGGTCCAGAAACTTGAG | NFARCWDNLVQKLEK |
| | | | AAGTCCACCGCCCAAATCAGCCAGGCCGTGACCACCACCCAGCCCTC | STAQISQAVTTTQPSLT |
| | | | TCTCACCCAAACCACCGTCATGGAGACAGTGACCACAGTGACTACCC | QTTVMETVTTVTTREQ |
| | | | GGGAGCAGATCCTTGTCAAACATGCTCAGGAGGAGCTTCCACCACCT | ILVKHAQEELPPPPPQK |
| | | | CCACCCCAAAAGAAGCGGCAGATCACCGTGGATAGCGAGATCCGCA | KRQITVDSEIRKRLDVD |
| | | | AGCGGCTTGACGTCGACATCACCGAGTTGCACTCTTGGATCACCCGGT | ITELHSWITRSEAVLQS |
| | | | CAGAGGCTGTCCTGCAGAGCCCAGAGTTCGCCATCTTCCGCAAGGAG | PEFAIFRKEGNFSDLKE |
| | | | GGCAACTTCTCCGACTTGAAAGAGAAGGTGAACGCTATCGAGCGGGA | KVNAIEREKAEKFRKL |
| | | | GAAGGCTGAGAAGTTCCGGAAGCTGCAGGACGCTAGCCGGTCCGCTC | QDASRSAQALVEQMV |
| | | | AGGCCCTCGTCGAGCAAATGGTCAATGAGGGCGTCAACGCAGACTCC | NEGVNADSIKQASEQL |
| | | | ATCAAGCAGGCATCCGAACAGCTCAACTCTCGGTGGATCGAGTTCTG | NSRWIEFCQLLSERLN |
| | | | CCAGCTCCTCTCCGAGCGGCTGAACTGGCTCGAGTACCAGAACAACA | WLEYQNNIIAFYNQLQ |
| | | | TTATCGCCTTCTACAATCAACTCCAACAGCTCGAGCAGATGACAACCA | QLEQMTTTAENWLKIQ |
| | | | CCGCTGAGAACTGGCTCAAGATCCAGCCAACCACACCAAGTGAGCCC | PTTPSEPTAIKSQLKICK |
| | | | ACCGCCATCAAGTCACAGTTGAAGATCTGCAAGGACGAGGTGAACAG | DEVNRLSDLQPQIERL |
| | | | ACTGTCCGATCTTCAGCCACAGATCGAGAGATTGAAGATCCAATCCA | KIQSIALKEKGQGPMFL |
| | | | TCGCACTTAAAGAGAAGGGCCAAGGGCCCATGTTCCTCGACGCCGAC | DADFVAFTNHFKQVFS |
| | | | TTTGTGGCTTTCACCAACCACTTCAAGCAGGTGTTTTCCGACGTTCAA | DVQAREKELQTIFDTLP |
| | | | GCCAGGGAGAAAGAGTTGCAGACCATCTTCGACACCCTCCCTCCCAT | PMRYQETMSAIRTWV |
| | | | GCGGTACCAAGAGACCATGAGCGCTATCCGCACCTGGGTGCAGCAAT | QQSETKLSIPQLSVTDY |
| | | | CCGAGACCAAGCTGAGCATCCCACAACTCTCCGTCACCGACTACGAG | EIMEQRLGELQALQSS |
| | | | ATCATGGAGCAGAGACTCGGCGAGCTCCAGGCTCTGCAGTCCTCTCT | LQEQQSGLYYLSTTVK |
| | | | GCAGGAACAACAGTCCGGGCTGTATTACCTCTCTACCACAGTGAAGG | EMSKKAPSEISRKYQSE |
| | | | AGATGTCCAAGAAGGCCCCATCCGAGATCAGCAGGAAGTACCAATCC | FEEIEGRWKKLSSQLV |
| | | | GAGTTCGAAGAGATCGAGGGCCGGTGGAAGAAGCTCTCCTCCCAGCT | EHCQKLEEQMNKLRKI |
| | | | GGTCGAGCACTGCCAGAAGCTCGAGGAGCAAATGAACAAGCTCCGG | QNHIQTLKKWMAEVD |
| | | | AAGATCCAGAACCACATCCAGACTTTGAAGAAGTGGATGGCTGAGGT | VFLKEEWPALGDSEIL |
| | | | GGACGTGTTCCTGAAGGAAGAATGGCCCGCACTCGGAGATTCCGAGA | KKQLKQCRLLVSDIQTI |
| | | | TCCTCAAGAAACAGCTGAAACAGTGCCGGCTCCTCGTGTCCGATATCC | QPSLNSVNEGGQKIKN |
| | | | AAACCATCCAGCCAAGCCTGAACTCCGTTAACGAGGGTGGCCAGAAA | EAEPEFASRLETELKEL |
| | | | ATCAAGAACGAGGCTGAGCCCGAGTTCGCCTCTCGGCTCGAGACTGA | NTQWDHMCQQVYAR |
| | | | GCTGAAAGAGTTGAATACCCAGTGGGACCACATGTGCCAGCAGGTGT | KEALKGGLEKTVSLQK |
| | | | ACGCTCGGAAGGAGGCTCTTAAGGGCGGGCTGGAGAAGACCGTCAG | DLSEMHEWMTQAEEE |
| | | | CCTCCAAAAGGACCTTAGCGAGATGCACGAGTGGATGACCCAAGCTG | YLERDFEYKTPDELQK |
| | | | AGGAGGAGTACCTCGAGCGCGACTTCGAGTACAAGACCCCTGACGAG | AVEEMKRAKEEAQQK |
| | | | CTCCAGAAGGCCGTGGAGGAGATGAAACGCGCAAAGGAAGAGGCCC | EAKVKLLTESVNSVIA |
| | | | AGCAGAAGGAAGCTAAAGTCAAGCTCCTCACCGAGTCCGTGAATTCC | QAPPVAQEALKKELET |
| | | | GTCATCGCCCAAGCACCACCCGTGGCCCAAGAAGCCCTCAAGAAGGA | LTTNYQWLCTRLNGK |
| | | | GCTCGAGACCCTCACTACCAACTACCAGTGGCTGTGCACCCGGCTGA | CKTLEEVWACWHELL |
| | | | ACGGCAAGTGTAAGACACTGGAAGAAGTGTGGGCCTGCTGGCATGAA | SYLEKANKWLNEVEF |
| | | | CTCCTCAGCTACCTGGAGAAGGCTAACAAGTGGCTCAACGAGGTGGA | KLKTTENIPGGAEEISE |
| | | | GTTCAAGCTGAAGACCACCGAGAACATTCCCGGTGGGGCCGAAGAAA | VLDSLENLMRHSEDNP |
| | | | TCTCCGAGGTCCTGGACAGCCTGGAGAACCTGATGCGGCATTCCGAG | NQIRILAQTLTDGGVM |
| | | | GACAACCCAAACCAAATCCGGATCCTGGCACAGACACTGACCGACGG | DELINEELETFNSRWRE |
| | | | AGGCGTCATGGACGAGCTCATCAACGAAGAGCTGGAGACATTTAACT | LHEEAVRRQKLLEQSI |
| | | | CCAGATGGCGGGAACTCCATGAGGAGGCCGTTAGACGGCAAAAGCTT | QSAQETEKSLHLIQESL |
| | | | CTGGAGCAATCCATCCAGAGCGCCCAGGAGACTGAGAAGAGTCTGCA | TFIDKQLAAYIADKVD |
| | | | CTTGATCCAGGAGAGTCTGACCTTCATCGACAAGCAACTCGCCGCCTA | AAQMPQEAQKIQSDLT |
| | | | CATCGCTGACAAGGTGGACGCCGCCCAAATGCCCCAGGAGGCTCAAA | SHEISLEEMKKHNQGK |
| | | | AGATTCAGTCAGACCTGACCAGCCACGAGATCAGCCTCGAAGAAATG | EAAQRVLSQIDVAQKK |
| | | | AAGAAGCACAACCAGGGAAAGGAGGCAGCCCAGAGGGTTCTGTCTC | LQDVSMKFRLFQKPAN |
| | | | AGATCGATGTGGCACAGAAGAAGCTTCAGGACGTGAGCATGAAGTTC | FEQRLQESKMILDEVK |
| | | | CGGCTGTTCCAAAAGCCCGCCAACTTCGAGCAGCGCCTCCAAGAGTC | MHLPALETKSVEQEVV |
| | | | CAAGATGATTCTCGACGAGGTCAAGATGCACCTGCCCGCCTTGGAGA | QSQLNHCVNLYKSLSE |
| | | | CCAAGTCTGTTGAACAGGAGGTGGTCCAGAGCCAGCTCAACCATTGC | VKSEVEMVIKTGRQIV |
| | | | GTCAACCTTTACAAGTCCCTCTCAGAGGTCAAGAGCGAAGTGGAGAT | QKKQTENPKELDERVT |
| | | | GGTTATCAAGACTGGGCGCCAGATCGTTCAAAAGAAGCAGACCGAGA | ALKLHYNELGAKVTER |
| | | | ACCCTAAGGAATTGGATGAACGGGTCACCGCCCTCAAGCTGCATTAC | KQQLEKCLKLSRKMR |
| | | | AACGAGCTCGGGGCCAAGGTCACCGAGAGAAAGCAGCAATTGGAGA | KEMNVLTEWLAATDM |
| | | | AGTGCCTCAAGCTGTCCCGGAAGATGCGCAAGGAGATGAACGTTCTG | ELTKRSAVEGMPSNLD |
| | | | ACCGAGTGGCTCGCAGCCACCGACATGGAGCTCACCAAGCGCTCCGC | SEVAWGKATQKEIEKQ |
| | | | TGTTGAGGGCATGCCCAGCAACCTGGATTCCGAAGTGGCTTGGGGCA | KVHLKSITEVGEALKT |
| | | | AGGCTACCCAGAAGGAAATCGAGAAGCAGAAGGTGCATCTGAAGTC | VLGKKETLVEDKLSLL |
| | | | CATTACCGAAGTCGGCGAGGCACTCAAAACCGTCCTCGGCAAGAAGG | NSNWIAVTSRAEEWLN |
| | | | AGACCCTGGTCGAAGACAAGTTGTCCCTTCTGAACTCCAACTGGATCG | LLLEYQKHMETFDQN |
| | | | CTGTCACCTCACGGGCCGAAGAGTGGCTGAATCTTCTGCTGGAGTATC | VDHITKWIIQADTLLDE |
| | | | AGAAACACATGGAGACCTTCGACCAGAACGTCGATCACATTACCAAG | SEKKKPQQKEDVLKRL |
| | | | TGGATCATCCAGGCCGATACATTGCTGGATGAGTCCGAGAAGAAGAA | KAELNDIRPKVDSTRD |
| | | | GCCCCAGCAGAAAGAGGATGTGCTGAAGCGGCTTAAGGCCGAGTTGA | QAANLMANRGDHCRK |
| | | | ACGACATCCGGCCCAAGGTCGACTCAACTCGCGACCAGGCCGCAAAC | LVEPQISELNHRFAAIS |
| | | | CTGATGGCCAATCGGGGCGATCACTGTCGGAAGCTGGTGGAACCCCA | HRIKTGKASIPLKELEQ |
| | | | GATCTCAGAGCTGAATCACAGGTTCGCCGCCATTTCCCACAGAATCA | FNSDIQKLLEPLEAEIQ |
| | | | AGACAGGCAAGGCAAGCATTCCTCTCAAGGAACTGGAGCAGTTCAAC | QGVNLKEEDFNKDMN |
| | | | AGCGACATCCAGAAACTCCTTGAACCACTGGAGGCTGAGATCCAGCA | EDNEGTVKELLQRGDN |
| | | | GGGTGTCAATCTGAAAGAAGAGGATTTCAACAAGGACATGAACGAG | LQQRITDERKREEIKIK |
| | | | GACAACGAGGGGACCGTCAAGGAGCTTCTGCAGCGCGGTGACAACCT | QQLLQTKHNALKDLRS |
| | | | CCAGCAACGGATCACCGATGAGAGAAAGCGGGAAGAGATCAAAATC | QRRKKALEISHQWYQ |
| | | | AAGCAGCAGCTCCTGCAGACCAAGCACAATGCTCTGAAGGACCTGAG | YKRQADDLLKCLDDIE |
| | | | ATCCCAGCGGCGGAAGAAGGCTCTGGAAATCTCCCACCAATGGTACC | KKLASLPEPRDERKIKE |
| | | | AGTACAAACGGCAGGCTGACGATCTGCTCAAGTGCCTGGACGACATC | IDRELQKKKEELNAVR |
| | | | GAAAAGAAGTTGGCCTCCCTGCCAGAGCCAAGGGACGAGAGAAAGA | RQAEGLSEDGAAMAV |
| | | | TCAAAGAGATCGACCGGGAGCTGCAAAAGAAGAAGGAAGAGCTGAA | EPTQIQLSKRWREIESK |
| | | | TGCAGTGAGGCGGCAAGCCGAGGGTCTGTCCGAAGACGGGGCTGCA | FAQFRRLNFAQIHTVR |
| | | | ATGGCTGTCGAGCCCACCCAGATTCAGCTGTCCAAGCGGTGGCGGGA | EETMMVMTEDMPLEIS |
| | | | GATCGAATCTAAGTTCGCCCAGTTCCGCAGACTCAACTTCGCCCAGAT | YVPSTYLTEITHVSQAL |
| | | | CCACACCGTGAGAGAGGAGACCATGATGGTGATGACCGAGGACATG | LEVEQLLNAPDLCAKD |
| | | | CCCCTGGAGATCAGTTACGTGCCCTCCACCTACCTGACCGAGATCACC | FEDLFKQEESLKNIKDS |
| | | | CACGTGTCACAGGCCCTGCTGGAGGTCGAGCAGCTCCTCAATGCTCC | LQQSSGRIDIIHSKKTA |
| | | | AGACCTCTGCGCCAAGGATTTCGAGGATCTGTTCAAGCAGGAGGAGT | ALQSATPVERVKLQEA |
| | | | CCCTGAAGAATATCAAGGACAGCCTGCAGCAGAGTAGCGGGCGGATC | LSQLDFQWEKVNKMY |
| | | | GACATCATCCACAGTAAGAAGACCGCTGCACTGCAATCCGCAACCCC | KDRQGRFDRSVEKWR |
| | | | AGTGGAGCGGGTCAAGTTGCAAGAGGCACTCAGCCAGCTGGACTTCC | RFHYDIKIFNQWLTEA |
| | | | AGTGGGAGAAGGTCAATAAAATGTACAAAGACCGGCAGGGCAGGTT | EQFLRKTQIPENWEHA |
| | | | CGACCGGTCCGTGGAGAAGTGGCGGCGGTTCCATTACGACATCAAGA | KYKWYLKELQDGIGQ |
| | | | TCTTTAACCAGTGGCTCACCGAAGCCGAGCAATTCCTGAGGAAGACC | RQTVVRTLNATGEEIIQ |
| | | | CAGATCCCAGAGAACTGGGAGCACGCCAAGTACAAGTGGTACCTGAA | QSSKTDASILQEKLGSL |
| | | | GGAATTGCAGGATGGTATCGGCCAACGGCAGACAGTCGTGCGGACAC | NLRWQEVCKQLSDRK |
| | | | TCAACGCTACCGGGGAGGAGATTATCCAGCAGAGCAGCAAGACCGA | KRLEEQKNILSEFQRDL |
| | | | CGCCTCCATTCTCCAGGAGAAGCTTGGTTCCCTGAACCTGCGGTGGCA | NEFVLWLEEADNIASIP |
| | | | GGAGGTCTGCAAGCAGCTCAGCGACAGGAAGAAGCGGCTGGAAGAG | LEPGKEQQLKEKLEQV |
| | | | CAGAAGAACATCCTGTCCGAGTTCCAACGGGATCTCAACGAGTTCGT | KLLVEELPLRQGILKQL |
| | | | CCTGTGGCTGGAGGAAGCTGACAACATCGCTTCCATCCCTCTGGAGCC | NETGGPVLVSAPISPEE |
| | | | CGGCAAAGAACAGCAGCTCAAGGAGAAGCTGGAGCAGGTGAAACTC | QDKLENKLKQTNLQWI |
| | | | CTGGTGGAGGAACTTCCCCTGCGGCAGGGTATCCTCAAGCAGCTGAA | KVSRALPEKQGEIEAQI |
| | | | TGAAACTGGTGGGCCCGTGCTTGTCTCCGCACCCATTTCCCCTGAGGA | KDLGQLEKKLEDLEEQ |
| | | | GCAGGACAAACTCGAGAACAAGTTGAAGCAGACCAACCTGCAGTGG | LNHLLLWLSPIRNQLEI |
| | | | ATCAAGGTCTCCCGGGCTCTTCCCGAGAAACAGGGCGAGATTGAGGC | YNQPNQEGPFDVKETE |
| | | | CCAGATCAAGGATCTCGGCCAGCTTGAGAAGAAGCTGGAAGATCTTG | IAVQAKQPDVEEILSKG |
| | | | AAGAGCAGTTGAACCATCTCTTGCTGTGGCTGTCCCCAATCCGGAACC | QHLYKEKPATQPVKRK |
| | | | AGCTGGAAATCTACAACCAGCCAAACCAGGAGGGGCCCTTCGACGTG | LEDLSSEWKAVNRLLQ |
| | | | AAGGAAACTGAGATCGCAGTGCAGGCCAAGCAGCCAGACGTCGAGG | ELRAKQPDLAPGLTTIG |
| | | | AGATCCTGTCCAAGGGGCAACACCTCTACAAAGAAAAGCCTGCAACT | ASPTQTVTLVTQPVVT |
| | | | CAGCCTGTCAAGCGGAAATTGGAGGACCTCTCCAGTGAGTGGAAGGC | KETAISKLEMPSSLMLE |
| | | | CGTCAACCGGCTCCTGCAAGAATTGCGCGCCAAACAACCCGACTTGG | VPALADFNRAWTELTD |
| | | | CACCAGGCCTGACTACAATCGGCGCATCACCCACCCAGACCGTGACC | WLSLLDQVIKSQRVMV |
| | | | CTGGTGACCCAGCCAGTGGTGACTAAAGAGACCGCCATCTCCAAGCT | GDLEDINEMIIKQKAT |
| | | | GGAGATGCCTTCCTCCCTCATGCTCGAGGTGCCAGCCCTTGCAGACTT | MQDLEQRRPQLEELIT |
| | | | CAACCGGGCATGGACAGAGCTCACTGATTGGCTGTCCCTCCTCGACC | AAQNLKNKTSNQEAR |
| | | | AGGTCATCAAGAGCCAACGGGTCATGGTTGGGGACCTGGAAGATATC | TIITDRIERIQNQWDEV |
| | | | AACGAGATGATCATCAAGCAGAAGGCTACCATGCAAGATCTCGAGCA | QEHLQNRRQQLNEML |
| | | | ACGGCGGCCACAGCTCGAAGAGCTCATCACTGCCGCCCAGAACCTCA | KDSTQWLEAKEEAEQ |
| | | | AGAATAAGACCTCCAACCAGGAAGCCCGGACTATCATCACAGACCGC | VLGQARAKLESWKEG |
| | | | ATCGAGCGCATCCAAAATCAGTGGGATGAGGTGCAGGAGCACCTCCA | PYTVDAIQKKITETKQL |
| | | | GAATCGGCGGCAACAGCTGAACGAAATGCTCAAGGATTCCACCCAGT | AKDLRQWQTNVDVAN |
| | | | GGCTTGAGGCTAAAGAGGAGGCAGAGCAAGTCCTGGGCCAAGCAAG | DLALKLLRDYSADDTR |
| | | | GGCAAAGCTGGAGTCCTGGAAGGAAGGCCCCTACACCGTCGACGCTA | KVHMITENINASWRSI |
| | | | TCCAGAAGAAGATCACCGAGACCAAACAGCTGGCAAAGGACTTGAG | HKRVSEREAALEETHR |
| | | | GCAGTGGCAGACCAATGTCGACGTGGCCAACGACCTCGCTCTTAAGC | LLQQFPLDLEKFLAWL |
| | | | TGCTGCGGGACTACTCCGCTGACGATACCCGGAAGGTCCACATGATC | TEAETTANVLQDATRK |
| | | | ACCGAAAATATCAACGCTTCCTGGCGGAGCATCCATAAGCGGGTGTC | ERLLEDSKGVKELMKQ |
| | | | AGAGCGCGAGGCAGCTCTGGAGGAGACCCATCGGCTGCTGCAACAGT | WQDLQGEIEAHTDVY |
| | | | TCCCTCTGGACCTCGAAAAGTTCCTGGCTTGGTTGACAGAAGCCGAG | HNLDENSQKILRSLEGS |
| | | | ACCACCGCTAACGTTCTGCAGGATGCCACCCGCAAGGAGAGATTGCT | DDAVLLQRRLDNMNF |
| | | | GGAGGACTCAAAGGGCGTGAAGGAGCTCATGAAGCAGTGGCAGGAC | KWSELRKKSLNIRSHL |
| | | | CTTCAGGGCGAAATCGAAGCTCACACAGACGTCTACCACAACCTCGA | EASSDQWKRLHLSLQE |
| | | | CGAGAACTCCCAAAAGATCCTGCGCTCCCTCGAAGGCAGCGACGACG | LLVWLQLKDDELSRQ |
| | | | CTGTGCTTCTGCAGAGGAGGCTGGACAACATGAACTTCAAGTGGTCC | APIGGDFPAVQKQNDV |
| | | | GAGCTGCGCAAGAAGTCCCTGAACATCCGGTCCCATTTGGAAGCTTC | HRAFKRELKTKEPVIM |
| | | | CTCCGACCAGTGGAAGCGGCTGCATCTCTCCCTGCAGGAACTGCTGGT | STLETVRIFLTEQPLEG |
| | | | GTGGCTCCAACTCAAGGACGATGAACTGAGCCGGCAGGCACCAATCG | LEKLYQEPRELPPEERA |
| | | | GAGGGGATTTCCCCGCCGTCCAGAAGCAGAACGATGTGCACCGGGCT | QNVTRLLRKQAEEVNT |
| | | | TTCAAGCGGGAGCTCAAGACCAAGGAGCCAGTGATCATGAGCACCCT | EWEKLNLHSADWQRK |
| | | | GGAGACCGTGCGGATCTTCCTGACAGAGCAGCCACTGGAAGGCCTCG | IDETLERLRELQEATDE |
| | | | AGAAGTTGTATCAGGAGCCTCGGGAACTCCCACCTGAAGAGCGCGCC | LDLKLRQAEVIKGSWQ |
| | | | CAGAACGTGACTCGGCTGCTCCGCAAGCAGGCAGAGGAGGTCAACAC | PVGDLLIDSLQDHLEK |
| | | | CGAATGGGAGAAGCTCAACCTGCATTCCGCCGACTGGCAGAGGAAGA | VKALRGEIAPLKENVS |
| | | | TCGATGAAACACTGGAGCGGCTCCAGGAGCTCCAAGAGGCAACCGAC | HVNDLARQLTTLGIQL |
| | | | GAGCTGGATCTGAAACTGAGACAGGCCGAAGTCATCAAGGGAAGCT | SPYNLSTLEDLNTRWK |
| | | | GGCAACCCGTTGGGGACTTGCTCATCGACTCCCTGCAAGACCATCTGG | LLQVAVEDRVRQLHE |
| | | | AGAAAGTGAAGGCACTGAGAGGCGAGATCGCACCACTGAAGGAGAA | AHRDFGPASQHFLSTS |
| | | | CGTCTCCCACGTGAATGATCTTGCCCGCCAGCTGACAACCCTGGGCAT | VQGPWERAISPNKVPY |
| | | | CCAACTCTCCCCATACAACCTCAGCACCCTCGAGGACCTGAACACCC | YINHETQTTCWDHPK |
| | | | GCTGGAAACTTCTGCAAGTGGCAGTCGAGGACAGGGTCAGGCAGCTC | MTELYQSLADLNNVRF |
| | | | CATGAGGCTCACCGCGACTTTGGACCTGCCAGCCAGCACTTCCTGTCT | SAYRTAMKLRRLQKA |
| | | | ACCTCCGTCCAAGGCCCATGGGAGAGAGCCATCTCTCCTAACAAGGT | LCLDLLSLSAACDALD |
| | | | GCCCTACTACATCAACCACGAGACTCAGACCACCTGCTGGGATCACC | QHNLKQNDQPMDILQI |
| | | | CCAAGATGACCGAACTGTATCAGTCCCTGGCTGATCTGAACAATGTCC | INCLTTIYDRLEQEHNN |
| | | | GCTTCTCCGCATACCGCACAGCCATGAAGCTTAGACGGCTGCAGAAG | LVNVPLCVDMCLNWL |
| | | | GCACTCTGCCTCGACCTGCTCTCTCTGTCAGCAGCCTGCGATGCACTC | LNVYDTGRTGRIRVLS |
| | | | GACCAGCACAACCTCAAGCAGAATGACCAGCCTATGGATATCCTGCA | FKTGIISLCKAHLEDKY |
| | | | GATCATCAACTGCCTGACCACCATCTACGATCGGCTCGAACAGGAGC | RYLFKQVASSTGFCDQ |
| | | | ACAACAATCTGGTGAACGTGCCTCTGTGCGTCGACATGTGCCTCAATT | RRLGLLLHDSIQIPRQL |
| | | | GGCTCCTGAACGTGTATGACACCGGGAGGACCGGAAGGATCCGGGTG | GEVASFGGSNIEPSVRS |
| | | | CTCAGCTTCAAGACAGGGATCATCAGCCTCTGCAAGGCCCATCTTGA | CFQFANNKPEIEAALFL |
| | | | GGATAAATACCGCTACCTCTTCAAACAAGTCGCCAGCTCTACCGGGTT | DWMRLEPQSMVWLPV |
| | | | CTGCGACCAACGCCGCTTGGGGCTTCTGCTCCACGATTCAATTCAAAT | LHRVAAAETAKHQAK |
| | | | CCCACGGCAGCTCGGTGAGGTGGCTTCCTTCGGCGGCTCCAACATCG | CNICKECPIIGFRYRSLK |
| | | | AGCCTTCTGTCCGCAGCTGCTTCCAGTTCGCCAACAACAAACCTGAGA | HFNYDICQSCFFSGRV |
| | | | TCGAGGCAGCCCTGTTCCTGGACTGGATGCGGCTCGAGCCTCAATCCA | AKGHKMHYPMVEYCT |
| | | | TGGTCTGGCTGCCCGTGCTCCATCGGGTCGCTGCTGCAGAGACAGCCA | PTTSGEDVRDFAKVLK |
| | | | AGCACCAGGCCAAGTGCAACATCTGTAAAGAGTGCCCAATCATTGGC | NKFRTKRYFAKHPRM |
| | | | TTCCGGTACAGATCCCTCAAGCACTTCAACTATGATATTTGCCAGAGC | GYLPVQTVLEGDNME |
| | | | TGCTTCTTCTCCGGCCGGGTTGCAAAGGGCCACAAGATGCACTATCCT | TPVTLINFWPVDSAPAS |
| | | | ATGGTGGAGTACTGCACCCCAACCACCTCCGGTGAAGACGTGCGGGA | SPQLSHDDTHSRIEHYA |
| | | | TTTCGCCAAGGTCCTTAAGAACAAGTTCCGGACCAAACGGTACTTCGC | SRLAEMENSNGSYLND |
| | | | CAAACACCCACGGATGGGGTACCTGCCCGTGCAGACCGTGCTCGAAG | SISPNESIDDEHLLIQHY |
| | | | GGGATAACATGGAAACCCCTGTCACCCTGATCAATTTCTGGCCAGTG | CQSLNQDSPLSQPRSPA |
| | | | GACTCTGCTCCAGCCAGCAGCCCACAGCTCTCACACGACGACACCCA | QILISLESEERGELERIL |
| | | | CTCCCGGATCGAGCACTATGCTAGCCGGCTGGCTGAGATGGAGAACT | ADLEEENRNLQAEYDR |
| | | | CCAACGGCTCCTACCTGAACGACTCAATCAGCCCCAACGAGAGCATC | LKQQHEHKGLSPLPSPP |
| | | | GACGACGAGCACCTGCTGATCCAGCACTACTGCCAGTCACTGAACCA | EMMPTSPQSPRDAELI |
| | | | GGACTCACCCCTCTCTCAGCCTAGGTCCCCAGCCCAGATCCTCATCTC | AEAKLLRQHKGRLEAR |
| | | | TCTGGAGTCTGAGGAGAGGGGCGAGCTCGAGAGGATCCTTGCCGACC | MQILEDHNKQLESQLH |
| | | | TGGAGGAGGAGAACCGGAACCTGCAGGCTGAGTACGACAGGCTGAA | RLRQLLEQPQAEAKVN |
| | | | ACAGCAGCACGAGCACAAGGGGCTGTCTCCACTCCCTTCACCTCCTG | GTTVSSPSTSLQRSDSS |
| | | | AGATGATGCCCACAAGCCCTCAGAGCCCTAGAGACGCCGAGCTGATC | QPMLLRVVGSQTSDSM |
| | | | GCCGAGGCCAAGCTGCTGAGACAGCACAAGGGCAGGCTGGAAGCTC | GEEDLLSPPQDTSTGLE |
| | | | GGATGCAGATCTTGGAAGACCACAACAAGCAGCTGGAGTCTCAGCTC | EVMEQLNNSFPSSRGR |
| | | | CACAGACTGCGGCAGCTTCTTGAGCAGCCACAGGCTGAGGCCAAGGT | NTPGKPMREDTM (SEQ |
| | | | GAACGGCACCACCGTCAGCTCTCCCAGCACCTCCCTGCAACGGTCTG | ID NO: 12) |
| | | | ATTCCAGCCAGCCCATGCTGCTCCGGGTTGTGGGCTCCCAGACCTCCG | |
| | | | ACTCCATGGGCGAGGAAGACTTGCTGTCACCACCACAGGATACCAGC | |
| | | | ACCGGTCTCGAAGAGGTGATGGAACAACTGAACAACTCCTTCCCCTC | |
| | | | TTCCCGGGGCCGGAACACACCCGGTAAGCCCATGCGCGAGGACACCA | |
| | | | TG (SEQ ID NO: 11) | |
| Full | Wild Type | NP_003997 | ATGCTGTGGTGGGAGGAGGTCGAGGACTGTTACGAGCGTGAGGACGT | MLWWEEVEDCYERED |
| Length | protein | | CCAAAAGAAGACCTTTACCAAGTGGGTGAACGCCCAGTTCAGCAAGT | VQKKTFTKWVNAQFS |
| human | (CO | | TCGGCAAGCAGCACATCGAGAATCTGTTCTCCGACTTACAAGACGGC | KFGKQHIENLFSDLQD |
| dystrophin | nucleotide | | CGGCGGCTACTTGACCTACTGGAGGGACTGACCGGCCAAAAGCTGCC | GRRLLDLLEGLTGQKL |
| | seq-3A) | | CAAGGAGAAGGGGAGCACTCGGGTGCACGCTCTGAACAACGTCAAC | PKEKGSTRVHALNNVN |
| | | | AAGGCCCTGCGTGTGCTGCAGAACAACAACGTGGACTTAGTGAACAT | KALRVLQNNNVDLVNI |
| | | | CGGCAGCACCGACATCGTGGACGGCAATCACAAGCTGACCTTAGGAC | GSTDIVDGNHKLTLGLI |
| | | | TGATCTGGAACATCATCCTGCACTGGCAGGTGAAGAACGTGATGAAG | WNIILHWQVKNVMKN |
| | | | AATATCATGGCCGGACTGCAGCAGACCAATAGCGAGAAGATACTCCT | IMAGLQQTNSEKILLS |
| | | | GAGCTGGGTGCGTCAAAGCACCCGTAACTACCCTCAGGTGAACGTCA | WVRQSTRNYPQVNVIN |
| | | | TCAACTTTACTACTAGCTGGAGCGACGGCCTTGCCTTAAACGCCCTGA | FTTSWSDGLALNALIH |
| | | | TCCACTCCCACCGGCCTGACCTGTTCGACTGGAACAGCGTGGTGTGTC | SHRPDLFDWNSVVCQ |
| | | | AACAGTCCGCCACACAGAGGCTGGAGCACGCCTTCAACATCGCCAGG | QSATQRLEHAFNIARY |
| | | | TACCAGCTGGGCATCGAGAAGCTACTGGACCCCGAGGACGTCGACAC | QLGIEKLLDPEDVDTT |
| | | | CACCTACCCCGACAAGAAGAGCATCTTAATGTACATCACCAGCCTGTT | YPDKKSILMYITSLFQV |
| | | | CCAAGTGCTGCCACAGCAGGTGAGCATCGAGGCCATCCAAGAGGTCG | LPQQVSIEAIQEVEMLP |
| | | | AGATGTTACCCCGGCCACCTAAGGTGACTAAGGAGGAGCACTTCCAG | RPPKVTKEEHFQLHHQ |
| | | | CTGCACCACCAGATGCACTACAGCCAGCAGATAACCGTGTCCCTTGC | MHYSQQITVSLAQGYE |
| | | | CCAGGGCTACGAGAGGACTAGCAGCCCAAAGCCACGGTTCAAGAGCT | RTSSPKPRFKSYAYTQ |
| | | | ACGCCTACACACAGGCCGCCTACGTGACAACCAGCGATCCCACCAGG | AAYVTTSDPTRSPFPSQ |
| | | | AGCCCCTTCCCCTCACAGCACCTGGAGGCACCCGAGGACAAGAGCTT | HLEAPEDKSFGSSLME |
| | | | CGGCTCATCCCTGATGGAGTCAGAGGTGAACCTGGACCGGTACCAGA | SEVNLDRYQTALEEVL |
| | | | CAGCCCTTGAGGAGGTCCTGTCATGGCTACTGAGCGCCGAGGACACT | SWLLSAEDTLQAQGEI |
| | | | CTGCAGGCCCAGGGTGAGATAAGCAATGACGTGGAGGTGGTGAAGG | SNDVEVVKDQFHTHE |
| | | | ACCAGTTCCACACCCACGAGGGCTACATGATGGACCTAACAGCTCAC | GYMMDLTAHQGRVG |
| | | | CAGGGTCGGGTCGGAAACATCCTTCAACTGGGCTCAAAGCTGATCGG | NILQLGSKLIGTGKLSE |
| | | | CACCGGCAAGCTTAGCGAGGACGAGGAGACCGAGGTCCAAGAGCAA | DEETEVQEQMNLLNSR |
| | | | ATGAATCTGCTGAACTCAAGGTGGGAGTGCCTGCGTGTGGCCTCCAT | WECLRVASMEKQSNL |
| | | | GGAGAAGCAGTCAAACCTTCACAGGGTGCTTATGGACCTGCAAAACC | HRVLMDLQNQKLKEL |
| | | | AGAAGCTCAAAGAGCTTAATGACTGGCTGACCAAAACTGAGGAGCG | NDWLTKTEERTRKME |
| | | | GACCAGGAAGATGGAGGAGGAGCCCCTAGGACCTGATCTTGAGGATT | EEPLGPDLEDLKRQVQ |
| | | | TAAAGCGGCAGGTCCAACAGCACAAGGTGTTACAAGAGGATTTAGAG | QHKVLQEDLEQEQVR |
| | | | CAAGAGCAGGTGCGGGTGAACTCCCTGACCCACATGGTGGTGGTGGT | VNSLTHMVVVVDESS |
| | | | GGATGAGTCAAGCGGAGACCACGCTACTGCCGCCCTTGAGGAGCAAC | GDHATAALEEQLKVL |
| | | | TCAAAGTGTTAGGCGACCGGTGGGCTAACATCTGCCGGTGGACAGAG | GDRWANICRWTEDRW |
| | | | GACCGGTGGGTGCTACTGCAAGATATTCTGCTGAAGTGGCAGCGGTT | VLLQDILLKWQRLTEE |
| | | | AACAGAGGAGCAGTGCCTGTTTTCCGCCTGGCTGAGCGAGAAGGAGG | QCLFSAWLSEKEDAVN |
| | | | ATGCCGTGAACAAAATCCACACCACCGGCTTCAAGGACCAAAATGAG | KIHTTGFKDQNEMLSS |
| | | | ATGCTCTCATCCTTACAGAAGCTAGCAGTGCTAAAGGCCGACTTAGA | LQKLAVLKADLEKKK |
| | | | GAAGAAGAAGCAAAGCATGGGCAAGCTGTACAGCCTCAAGCAGGAC | QSMGKLYSLKQDLLST |
| | | | TTACTTAGCACACTCAAGAATAAGAGCGTGACCCAGAAGACCGAGGC | LKNKSVTQKTEAWLD |
| | | | CTGGTTAGACAACTTCGCCCGGTGCTGGGACAACCTTGTGCAGAAAC | NFARCWDNLVQKLEK |
| | | | TCGAGAAGAGCACCGCCCAAATTTCACAGGCCGTGACTACCACCCAG | STAQISQAVTTTQPSLT |
| | | | CCAAGCCTCACCCAGACCACCGTCATGGAGACTGTCACCACTGTGAC | QTTVMETVTTVTTREQ |
| | | | CACCCGGGAGCAGATCCTCGTGAAGCACGCCCAAGAGGAGCTGCCAC | ILVKHAQEELPPPPPQK |
| | | | CTCCTCCACCCCAGAAGAAGCGGCAGATCACCGTCGACTCCGAGATC | KRQITVDSEIRKRLDVD |
| | | | CGTAAGCGGCTGGATGTGGACATCACCGAGCTGCACAGCTGGATCAC | ITELHSWITRSEAVLQS |
| | | | CAGGTCCGAGGCCGTCCTACAAAGCCCCGAGTTTGCCATCTTTCGGAA | PEFAIFRKEGNFSDLKE |
| | | | GGAGGGTAACTTTAGCGACCTCAAAGAGAAAGTGAACGCCATCGAGC | KVNAIEREKAEKFRKL |
| | | | GGGAGAAGGCCGAGAAGTTCCGGAAGCTACAGGACGCCTCACGGTC | QDASRSAQALVEQMV |
| | | | AGCCCAGGCTCTGGTCGAGCAAATGGTGAACGAGGGAGTGAACGCC | NEGVNADSIKQASEQL |
| | | | GACTCAATCAAGCAGGCCAGCGAGCAGCTGAATTCACGGTGGATCGA | NSRWIEFCQLLSERLN |
| | | | GTTTTGTCAGCTGCTGTCAGAGAGGCTCAACTGGCTCGAGTACCAAA | WLEYQNNIIAFYNQLQ |
| | | | ACAACATCATCGCCTTCTACAACCAACTTCAGCAGCTCGAGCAGATG | QLEQMTTTAENWLKIQ |
| | | | ACCACCACCGCTGAGAACTGGCTGAAAATCCAGCCAACCACTCCAAG | PTTPSEPTAIKSQLKICK |
| | | | CGAGCCTACCGCCATCAAGTCACAGCTGAAGATTTGCAAAGACGAGG | DEVNRLSDLQPQIERL |
| | | | TGAACCGGCTGAGCGATCTGCAACCCCAGATCGAGCGGCTCAAGATC | KIQSIALKEKGQGPMFL |
| | | | CAGAGCATTGCCTTAAAGGAGAAAGGCCAAGGCCCCATGTTCCTAGA | DADFVAFTNHFKQVFS |
| | | | CGCCGACTTCGTGGCCTTCACCAACCACTTTAAGCAAGTGTTCAGCGA | DVQAREKELQTIFDTLP |
| | | | TGTGCAAGCCCGGGAGAAGGAGCTACAGACCATCTTCGATACCCTGC | PMRYQETMSAIRTWV |
| | | | CTCCCATGCGGTACCAGGAGACAATGAGCGCCATCCGGACATGGGTC | QQSETKLSIPQLSVTDY |
| | | | CAGCAGTCAGAGACCAAGCTGAGCATTCCACAACTGAGCGTGACCGA | EIMEQRLGELQALQSS |
| | | | CTACGAGATCATGGAGCAGAGGTTAGGCGAGCTGCAGGCTCTTCAGA | LQEQQSGLYYLSTTVK |
| | | | GCAGCCTGCAAGAGCAACAAAGCGGCCTGTACTACCTGAGCACCACC | EMSKKAPSEISRKYQSE |
| | | | GTCAAGGAGATGAGCAAGAAGGCCCCTTCCGAGATTAGCCGGAAGTA | FEEIEGRWKKLSSQLV |
| | | | CCAGAGCGAGTTCGAGGAGATCGAGGGCCGGTGGAAGAAGCTGTCC | EHCQKLEEQMNKLRKI |
| | | | AGCCAGCTAGTGGAGCACTGCCAGAAGCTTGAGGAGCAAATGAACA | QNHIQTLKKWMAEVD |
| | | | AACTGCGGAAAATCCAGAATCACATTCAGACCCTGAAGAAGTGGATG | VFLKEEWPALGDSEIL |
| | | | GCCGAGGTGGACGTGTTCCTTAAGGAGGAGTGGCCCGCTCTTGGCGA | KKQLKQCRLLVSDIQTI |
| | | | CAGCGAGATACTCAAGAAGCAACTTAAACAGTGCCGGTTACTGGTGA | QPSLNSVNEGGQKIKN |
| | | | GCGACATACAAACCATCCAGCCTAGCCTGAACAGCGTCAACGAGGGT | EAEPEFASRLETELKEL |
| | | | GGCCAGAAAATCAAGAACGAGGCTGAGCCCGAGTTCGCAAGCCGGC | NTQWDHMCQQVYAR |
| | | | TGGAGACCGAGCTTAAAGAGCTCAACACCCAGTGGGACCACATGTGT | KEALKGGLEKTVSLQK |
| | | | CAGCAGGTGTACGCCCGTAAAGAGGCACTTAAAGGCGGACTGGAGA | DLSEMHEWMTQAEEE |
| | | | AGACTGTGTCACTGCAGAAGGATCTGAGCGAGATGCACGAGTGGATG | YLERDFEYKTPDELQK |
| | | | ACCCAAGCTGAGGAGGAGTACCTGGAGAGGGACTTCGAGTACAAGA | AVEEMKRAKEEAQQK |
| | | | CCCCAGACGAGCTGCAGAAAGCCGTCGAGGAGATGAAACGGGCCAA | EAKVKLLTESVNSVIA |
| | | | AGAGGAGGCTCAGCAGAAAGAGGCAAAAGTGAAGCTACTGACCGAG | QAPPVAQEALKKELET |
| | | | AGCGTGAATAGCGTGATCGCCCAAGCTCCACCCGTGGCTCAGGAGGC | LTTNYQWLCTRLNGK |
| | | | ACTGAAGAAGGAGTTAGAGACACTGACCACCAACTACCAATGGCTTT | CKTLEEVWACWHELL |
| | | | GCACCCGGCTGAACGGCAAGTGCAAGACACTAGAGGAGGTGTGGGC | SYLEKANKWLNEVEF |
| | | | ATGCTGGCACGAGCTGCTGAGCTACCTCGAGAAAGCCAACAAGTGGC | KLKTTENIPGGAEEISE |
| | | | TGAATGAGGTGGAGTTCAAGCTAAAGACCACCGAGAATATACCTGGT | VLDSLENLMRHSEDNP |
| | | | GGCGCAGAGGAGATCAGCGAGGTGCTGGACAGCCTAGAGAACCTGA | NQIRILAQTLTDGGVM |
| | | | TGCGGCACAGCGAGGACAACCCCAACCAGATCCGTATCCTGGCCCAG | DELINEELETFNSRWRE |
| | | | ACCCTAACTGATGGCGGCGTGATGGACGAGCTGATCAACGAGGAGCT | LHEEAVRRQKLLEQSI |
| | | | CGAGACCTTCAATAGCAGGTGGCGGGAGTTACACGAGGAGGCCGTGC | QSAQETEKSLHLIQESL |
| | | | GTCGGCAAAAGCTACTTGAGCAGAGCATCCAAAGCGCCCAGGAGAC | TFIDKQLAAYIADKVD |
| | | | AGAGAAGTCCCTGCACCTGATCCAGGAGTCACTCACCTTCATCGACA | AAQMPQEAQKIQSDLT |
| | | | AGCAGCTAGCCGCCTACATCGCCGACAAGGTGGACGCAGCCCAGATG | SHEISLEEMKKHNQGK |
| | | | CCCCAGGAGGCACAGAAGATCCAGTCAGACCTGACCAGCCACGAGAT | EAAQRVLSQIDVAQKK |
| | | | CAGCCTTGAGGAGATGAAGAAGCACAACCAGGGCAAAGAGGCAGCC | LQDVSMKFRLFQKPAN |
| | | | CAACGGGTGCTAAGCCAGATCGACGTGGCCCAGAAGAAACTGCAGG | FEQRLQESKMILDEVK |
| | | | ACGTGAGCATGAAGTTCCGGCTGTTCCAAAAGCCCGCCAACTTCGAG | MHLPALETKSVEQEVV |
| | | | CAGCGGCTTCAGGAGAGCAAGATGATCTTAGATGAGGTCAAGATGCA | QSQLNHCVNLYKSLSE |
| | | | CCTGCCAGCCTTAGAGACCAAGTCAGTGGAGCAAGAGGTGGTGCAGA | VKSEVEMVIKTGRQIV |
| | | | GCCAGCTTAACCACTGTGTGAACCTTTACAAGAGCCTTAGCGAGGTC | QKKQTENPKELDERVT |
| | | | AAAAGCGAGGTGGAGATGGTGATAAAGACAGGCCGGCAGATCGTGC | ALKLHYNELGAKVTER |
| | | | AAAAGAAGCAGACCGAGAACCCCAAGGAGCTTGACGAGCGGGTCAC | KQQLEKCLKLSRKMR |
| | | | TGCCCTTAAGTTACACTACAACGAGCTGGGCGCCAAGGTCACCGAGC | KEMNVLTEWLAATDM |
| | | | GGAAACAGCAGCTAGAGAAATGCCTGAAGCTCTCCCGGAAGATGCG | ELTKRSAVEGMPSNLD |
| | | | GAAAGAGATGAACGTGCTAACCGAGTGGCTGGCAGCCACCGACATG | SEVAWGKATQKEIEKQ |
| | | | GAGCTGACCAAGAGGTCAGCTGTGGAGGGCATGCCCAGCAACCTCGA | KVHLKSITEVGEALKT |
| | | | CAGCGAGGTCGCATGGGGCAAGGCCACCCAGAAAGAGATAGAGAAG | VLGKKETLVEDKLSLL |
| | | | CAGAAGGTGCACCTGAAGAGCATCACCGAGGTGGGCGAGGCCCTAA | NSNWIAVTSRAEEWLN |
| | | | AGACTGTGCTGGGCAAGAAGGAGACATTAGTCGAGGACAAACTGAG | LLLEYQKHMETFDQN |
| | | | CCTTCTGAACAGCAACTGGATCGCAGTGACTAGCCGGGCAGAGGAGT | VDHITKWIIQADTLLDE |
| | | | GGTTAAACCTGCTACTGGAGTACCAGAAGCACATGGAGACTTTTGAC | SEKKKPQQKEDVLKRL |
| | | | CAGAACGTGGACCACATCACCAAGTGGATCATCCAGGCTGACACCCT | KAELNDIRPKVDSTRD |
| | | | GCTCGACGAGAGCGAGAAGAAGAAGCCCCAACAGAAAGAGGATGTG | QAANLMANRGDHCRK |
| | | | CTGAAGCGGCTGAAGGCAGAGCTGAACGATATCCGGCCCAAGGTCGA | LVEPQISELNHRFAAIS |
| | | | TAGCACCCGTGACCAGGCAGCCAACCTGATGGCCAACCGGGGCGATC | HRIKTGKASIPLKELEQ |
| | | | ACTGTCGGAAGCTGGTGGAGCCCCAAATCAGCGAGCTCAACCACCGG | FNSDIQKLLEPLEAEIQ |
| | | | TTCGCTGCCATCAGCCACCGGATCAAGACAGGCAAGGCCAGCATTCC | QGVNLKEEDFNKDMN |
| | | | ATTAAAGGAGCTAGAGCAATTCAACTCCGACATCCAGAAACTGCTAG | EDNEGTVKELLQRGDN |
| | | | AGCCCCTAGAGGCTGAGATCCAACAGGGCGTGAATCTGAAGGAGGA | LQQRITDERKREEIKIK |
| | | | GGACTTCAACAAGGACATGAACGAGGACAACGAGGGCACAGTCAAG | QQLLQTKHNALKDLRS |
| | | | GAGCTGCTGCAACGGGGCGACAACCTGCAACAGCGGATCACCGACG | QRRKKALEISHQWYQ |
| | | | AGAGGAAGCGTGAGGAGATCAAGATCAAGCAACAGCTTCTGCAAAC | YKRQADDLLKCLDDIE |
| | | | CAAGCACAATGCCCTTAAGGACCTACGGAGCCAACGGCGGAAGAAG | KKLASLPEPRDERKIKE |
| | | | GCCCTGGAGATCAGCCACCAGTGGTACCAGTACAAGCGGCAGGCAGA | IDRELQKKKEELNAVR |
| | | | CGACCTACTGAAGTGCCTGGACGACATTGAGAAGAAGTTAGCCAGCC | RQAEGLSEDGAAMAV |
| | | | TGCCTGAGCCAAGGGACGAGCGGAAAATCAAGGAGATCGACCGGGA | EPTQIQLSKRWREIESK |
| | | | GCTTCAGAAGAAGAAGGAGGAGCTTAACGCAGTCCGGCGGCAGGCC | FAQFRRLNFAQIHTVR |
| | | | GAGGGTCTCTCAGAGGATGGCGCTGCTATGGCAGTGGAGCCCACCCA | EETMMVMTEDMPLEIS |
| | | | GATCCAACTGAGCAAGCGTTGGCGTGAGATAGAGAGCAAGTTCGCCC | YVPSTYLTEITHVSQAL |
| | | | AGTTCCGTCGGCTGAACTTCGCCCAGATCCACACAGTGAGGGAGGAG | LEVEQLLNAPDLCAKD |
| | | | ACTATGATGGTGATGACTGAGGACATGCCCCTGGAGATTAGCTACGT | FEDLFKQEESLKNIKDS |
| | | | GCCCAGCACCTACCTGACCGAGATCACACACGTGTCCCAGGCCCTTCT | LQQSSGRIDIIHSKKTA |
| | | | GGAGGTGGAGCAGCTCTTAAACGCTCCCGACCTGTGTGCCAAGGACT | ALQSATPVERVKLQEA |
| | | | TCGAGGACCTGTTTAAGCAGGAGGAGAGCCTTAAGAACATCAAGGAC | LSQLDFQWEKVNKMY |
| | | | TCCTTACAGCAAAGCAGCGGCCGGATCGACATCATCCACAGCAAGAA | KDRQGRFDRSVEKWR |
| | | | GACCGCTGCCCTACAGAGCGCCACACCTGTGGAGCGTGTCAAACTAC | RFHYDIKIFNQWLTEA |
| | | | AAGAGGCACTGAGCCAACTGGACTTCCAATGGGAGAAGGTCAATAA | EQFLRKTQIPENWEHA |
| | | | GATGTACAAGGACCGTCAGGGCAGGTTCGACCGGAGCGTGGAGAAG | KYKWYLKELQDGIGQ |
| | | | TGGCGGCGGTTCCACTACGACATTAAGATCTTCAACCAGTGGTTAACT | RQTVVRTLNATGEEIIQ |
| | | | GAGGCAGAGCAGTTCCTGCGGAAGACCCAGATCCCAGAGAACTGGG | QSSKTDASILQEKLGSL |
| | | | AGCACGCCAAGTACAAGTGGTACCTGAAGGAGCTGCAAGACGGCAT | NLRWQEVCKQLSDRK |
| | | | AGGTCAGCGTCAGACCGTCGTCCGGACCCTAAACGCCACCGGCGAGG | KRLEEQKNILSEFQRDL |
| | | | AGATCATCCAACAGAGCAGCAAGACCGACGCCAGCATTCTGCAGGAG | NEFVLWLEEADNIASIP |
| | | | AAACTGGGCAGCCTGAACCTGCGGTGGCAGGAGGTGTGCAAGCAGCT | LEPGKEQQLKEKLEQV |
| | | | AAGCGACCGGAAGAAAAGGCTTGAGGAGCAAAAGAACATCCTGAGC | KLLVEELPLRQGILKQL |
| | | | GAGTTCCAGCGGGACCTGAACGAGTTCGTGCTGTGGCTAGAGGAGGC | NETGGPVLVSAPISPEE |
| | | | CGACAACATCGCCTCAATACCCCTGGAGCCCGGCAAAGAGCAACAGC | QDKLENKLKQTNLQWI |
| | | | TCAAGGAGAAACTTGAGCAGGTCAAGCTGCTAGTGGAGGAGCTTCCA | KVSRALPEKQGEIEAQI |
| | | | CTGCGGCAGGGCATCTTAAAGCAGCTAAACGAGACTGGTGGCCCCGT | KDLGQLEKKLEDLEEQ |
| | | | CTTAGTGAGCGCCCCTATCAGCCCTGAGGAGCAGGATAAGCTAGAGA | LNHLLLWLSPIRNQLEI |
| | | | ACAAGCTGAAGCAAACCAATCTGCAGTGGATCAAAGTGTCACGGGCC | YNQPNQEGPFDVKETE |
| | | | CTGCCAGAGAAGCAAGGGGAGATAGAGGCTCAGATCAAAGACCTGG | IAVQAKQPDVEEILSKG |
| | | | GTCAGCTCGAGAAGAAGTTAGAGGACCTAGAGGAGCAGCTAAATCA | QHLYKEKPATQPVKRK |
| | | | CCTCTTACTGTGGCTGAGCCCAATCCGGAACCAACTGGAGATCTACA | LEDLSSEWKAVNRLLQ |
| | | | ATCAGCCTAACCAAGAGGGTCCCTTCGACGTGAAAGAGACAGAGATC | ELRAKQPDLAPGLTTIG |
| | | | GCCGTGCAAGCCAAACAACCCGATGTGGAGGAGATCCTGAGCAAGG | ASPTQTVTLVTQPVVT |
| | | | GCCAACACCTGTACAAAGAGAAGCCAGCAACCCAACCCGTGAAGCG | KETAISKLEMPSSLMLE |
| | | | GAAACTGGAGGACCTCAGCAGCGAGTGGAAGGCCGTGAACAGGTTA | VPALADFNRAWTELTD |
| | | | CTGCAGGAGCTGCGTGCCAAGCAGCCTGACCTAGCACCCGGCCTGAC | WLSLLDQVIKSQRVMV |
| | | | TACCATAGGCGCCAGCCCTACCCAAACTGTGACCCTAGTGACCCAGC | GDLEDINEMIIKQKAT |
| | | | CCGTCGTGACCAAGGAGACCGCCATTAGCAAACTGGAGATGCCATCA | MQDLEQRRPQLEELIT |
| | | | AGCCTGATGCTGGAGGTGCCCGCCTTAGCCGACTTCAATCGGGCCTG | AAQNLKNKTSNQEAR |
| | | | GACCGAGCTCACAGACTGGCTGTCCCTGCTGGACCAGGTCATCAAGA | TIITDRIERIQNQWDEV |
| | | | GCCAACGGGTGATGGTGGGAGATCTGGAGGACATCAACGAGATGATC | QEHLQNRRQQLNEML |
| | | | ATCAAGCAGAAGGCCACCATGCAAGACCTAGAGCAGCGTCGGCCCCA | KDSTQWLEAKEEAEQ |
| | | | ATTAGAGGAGCTGATCACCGCAGCACAAAACCTGAAGAACAAGACC | VLGQARAKLESWKEG |
| | | | AGCAATCAGGAGGCTCGGACCATCATCACCGACCGGATTGAGCGGAT | PYTVDAIQKKITETKQL |
| | | | TCAGAACCAGTGGGATGAGGTGCAAGAGCACCTTCAGAACCGTCGGC | AKDLRQWQTNVDVAN |
| | | | AACAGCTTAACGAGATGCTGAAGGACAGCACCCAATGGCTTGAGGCA | DLALKLLRDYSADDTR |
| | | | AAGGAGGAGGCTGAGCAAGTCCTCGGTCAGGCTAGGGCCAAGCTTGA | KVHMITENINASWRSI |
| | | | GAGCTGGAAAGAGGGGCCCTACACCGTGGACGCCATCCAGAAGAAG | HKRVSEREAALEETHR |
| | | | ATCACTGAGACCAAACAGCTGGCCAAAGACCTGCGGCAGTGGCAGAC | LLQQFPLDLEKFLAWL |
| | | | CAACGTGGACGTGGCCAACGACCTGGCATTAAAGTTACTTCGGGACT | TEAETTANVLQDATRK |
| | | | ACAGCGCAGACGACACACGGAAGGTGCACATGATTACCGAGAACAT | ERLLEDSKGVKELMKQ |
| | | | CAACGCCTCATGGCGGAGCATCCACAAGCGGGTGAGCGAGAGGGAG | WQDLQGEIEAHTDVY |
| | | | GCTGCTCTTGAGGAGACCCACCGGCTCCTGCAACAGTTCCCACTGGAC | HNLDENSQKILRSLEGS |
| | | | CTTGAGAAATTCCTGGCATGGCTGACCGAGGCAGAGACCACCGCCAA | DDAVLLQRRLDNMNF |
| | | | CGTGCTCCAAGACGCCACCCGTAAGGAGCGGCTACTGGAGGACAGCA | KWSELRKKSLNIRSHL |
| | | | AGGGAGTGAAAGAGCTAATGAAGCAGTGGCAGGACCTCCAAGGCGA | EASSDQWKRLHLSLQE |
| | | | GATCGAGGCCCACACCGACGTGTACCACAACCTGGACGAGAACAGCC | LLVWLQLKDDELSRQ |
| | | | AGAAGATTCTCCGGAGCCTGGAGGGATCCGACGACGCCGTCCTCCTG | APIGGDFPAVQKQNDV |
| | | | CAGAGGAGGCTGGACAACATGAACTTCAAGTGGAGCGAGCTGCGGA | HRAFKRELKTKEPVIM |
| | | | AGAAGAGCCTCAACATCCGGAGCCACCTAGAGGCCAGCAGCGACCA | STLETVRIFLTEQPLEG |
| | | | GTGGAAACGGCTGCACCTGTCACTGCAAGAGCTTCTTGTGTGGCTGCA | LEKLYQEPRELPPEERA |
| | | | GCTGAAAGACGACGAGCTGTCACGGCAGGCACCCATAGGTGGCGATT | QNVTRLLRKQAEEVNT |
| | | | TCCCCGCCGTGCAGAAGCAGAATGACGTGCACCGGGCCTTCAAGCGG | EWEKLNLHSADWQRK |
| | | | GAGCTGAAAACCAAGGAGCCCGTGATCATGAGCACCCTAGAGACCGT | IDETLERLRELQEATDE |
| | | | CCGGATCTTCCTGACCGAGCAGCCCCTTGAGGGCCTAGAGAAACTGT | LDLKLRQAEVIKGSWQ |
| | | | ACCAGGAGCCTCGTGAGCTGCCTCCAGAGGAGAGGGCCCAGAACGTG | PVGDLLIDSLQDHLEK |
| | | | ACCCGGTTACTGCGGAAGCAGGCAGAGGAGGTGAACACCGAGTGGG | VKALRGEIAPLKENVS |
| | | | AGAAGCTGAATCTGCACAGCGCTGACTGGCAGCGGAAGATTGACGAG | HVNDLARQLTTLGIQL |
| | | | ACCCTCGAGCGGCTACAAGAGCTCCAGGAGGCCACCGATGAGCTAGA | SPYNLSTLEDLNTRWK |
| | | | CCTAAAGCTGCGTCAGGCCGAGGTGATTAAGGGCTCATGGCAGCCAG | LLQVAVEDRVRQLHE |
| | | | TCGGCGATCTTTTAATCGACTCCCTCCAAGACCACCTAGAGAAGGTGA | AHRDFGPASQHELSTS |
| | | | AGGCTTTACGGGGCGAGATAGCCCCTCTAAAGGAGAACGTGAGCCAC | VQGPWERAISPNKVPY |
| | | | GTGAATGACCTGGCACGGCAGCTGACCACCCTGGGCATCCAGCTAAG | YINHETQTTCWDHPK |
| | | | CCCCTACAACCTGTCAACACTCGAGGATCTAAACACCCGGTGGAAAC | MTELYQSLADLNNVRF |
| | | | TGCTGCAGGTCGCAGTGGAGGACAGGGTCCGGCAACTGCACGAGGCT | SAYRTAMKLRRLQKA |
| | | | CACCGGGACTTCGGACCTGCTAGCCAGCACTTCCTGTCCACTAGCGTG | LCLDLLSLSAACDALD |
| | | | CAAGGCCCTTGGGAGCGGGCAATTTCCCCAAACAAGGTGCCCTACTA | QHNLKQNDQPMDILQI |
| | | | CATCAACCACGAGACTCAGACAACCTGCTGGGACCACCCTAAGATGA | INCLTTIYDRLEQEHNN |
| | | | CCGAGCTGTACCAAAGCCTGGCCGACTTAAACAACGTGCGTTTCTCCG | LVNVPLCVDMCLNWL |
| | | | CTTACCGGACCGCCATGAAGTTACGTCGGCTGCAGAAGGCACTGTGC | LNVYDTGRTGRIRVLS |
| | | | CTAGACCTGCTGTCACTGTCCGCTGCCTGTGATGCCCTAGACCAGCAC | FKTGIISLCKAHLEDKY |
| | | | AACCTGAAGCAAAACGACCAGCCCATGGATATCCTGCAGATCATCAA | RYLFKQVASSTGFCDQ |
| | | | CTGCTTAACCACCATCTACGACCGTCTCGAGCAGGAGCACAACAACC | RRLGLLLHDSIQIPRQL |
| | | | TGGTCAACGTGCCCCTGTGCGTGGACATGTGCCTGAACTGGCTCCTGA | GEVASFGGSNIEPSVRS |
| | | | ATGTGTACGACACTGGACGTACTGGACGGATCCGGGTGCTTTCATTCA | CFQFANNKPEIEAALFL |
| | | | AAACCGGCATCATCAGCCTGTGCAAGGCTCACCTTGAGGACAAATAC | DWMRLEPQSMVWLPV |
| | | | CGGTACCTATTCAAGCAGGTCGCCTCAAGCACCGGCTTCTGCGACCA | LHRVAAAETAKHQAK |
| | | | GAGGCGGCTTGGACTGCTGCTGCACGATAGCATCCAGATACCCCGTC | CNICKECPIIGFRYRSLK |
| | | | AGTTAGGCGAGGTGGCAAGCTTCGGTGGCAGCAACATCGAGCCCTCA | HFNYDICQSCFFSGRV |
| | | | GTGCGGAGCTGCTTCCAGTTCGCCAACAACAAACCAGAGATTGAGGC | AKGHKMHYPMVEYCT |
| | | | AGCCTTATTCCTGGACTGGATGCGGCTTGAGCCCCAGAGCATGGTGTG | PTTSGEDVRDFAKVLK |
| | | | GCTCCCCGTGCTACACAGGGTGGCAGCTGCAGAGACCGCCAAACACC | NKFRTKRYFAKHPRM |
| | | | AGGCCAAGTGCAACATATGCAAGGAGTGCCCCATCATCGGCTTCCGT | GYLPVQTVLEGDNME |
| | | | TACCGGTCACTGAAGCACTTCAACTACGACATATGCCAGAGCTGCTTC | TPVTLINFWPVDSAPAS |
| | | | TTCTCCGGACGGGTGGCAAAGGGCCACAAAATGCACTACCCCATGGT | SPQLSHDDTHSRIEHYA |
| | | | GGAGTACTGTACCCCAACTACAAGCGGTGAGGACGTGCGGGACTTTG | SRLAEMENSNGSYLND |
| | | | CCAAGGTGCTGAAGAACAAGTTCCGGACAAAGCGGTACTTCGCTAAG | SISPNESIDDEHLLIQHY |
| | | | CACCCACGGATGGGCTACCTGCCCGTGCAGACCGTCCTGGAGGGCGA | CQSLNQDSPLSQPRSPA |
| | | | CAACATGGAGACACCCGTCACCCTCATTAACTTTTGGCCCGTGGATTC | QILISLESEERGELERIL |
| | | | AGCTCCAGCCAGCAGCCCTCAGCTGAGCCACGACGACACACACAGCC | ADLEEENRNLQAEYDR |
| | | | GTATCGAGCACTACGCTAGCCGTCTAGCCGAGATGGAGAACAGCAAC | LKQQHEHKGLSPLPSPP |
| | | | GGCAGCTACCTGAACGACAGCATAAGCCCCAACGAGAGCATCGACG | EMMPTSPQSPRDAELI |
| | | | ACGAGCACTTACTGATCCAGCACTACTGCCAAAGCCTGAACCAGGAC | AEAKLLRQHKGRLEAR |
| | | | AGCCCTCTGTCACAACCTCGGAGCCCCGCTCAAATTCTGATCAGCCTA | MQILEDHNKQLESQLH |
| | | | GAGTCAGAGGAGCGGGGCGAGTTAGAGCGGATACTGGCCGATTTAG | RLRQLLEQPQAEAKVN |
| | | | AGGAGGAGAACCGGAACCTGCAGGCCGAGTACGACCGGTTAAAACA | GTTVSSPSTSLQRSDSS |
| | | | GCAGCACGAGCACAAGGGGTTAAGCCCACTACCCAGCCCTCCCGAGA | QPMLLRVVGSQTSDSM |
| | | | TGATGCCCACCTCACCTCAATCCCCACGTGATGCCGAGCTGATCGCAG | GEEDLLSPPQDTSTGLE |
| | | | AGGCCAAGCTGCTGCGTCAGCACAAAGGGAGGCTTGAGGCCAGGAT | EVMEQLNNSFPSSRGR |
| | | | GCAGATCCTTGAGGACCACAACAAGCAACTCGAGAGCCAACTGCACC | NTPGKPMREDTM (SEQ |
| | | | GGCTGAGGCAACTGCTGGAGCAACCCCAAGCCGAGGCCAAAGTGAA | ID NO: 14) |
| | | | CGGCACTACCGTCTCATCACCCAGCACCTCCCTGCAGCGTAGCGACA | |
| | | | GCAGCCAGCCCATGCTTCTGAGGGTGGTGGGCAGCCAGACCAGCGAC | |
| | | | AGCATGGGCGAGGAGGACCTTCTGTCACCACCCCAGGACACTAGCAC | |
| | | | CGGACTGGAGGAGGTCATGGAGCAACTTAACAACAGCTTCCCTTCCA | |
| | | | GCCGTGGTCGGAATACACCCGGCAAGCCTATGCGGGAGGACACCATG | |
| | | | (SEQ ID NO: 13) | |

| | | | | |
|---|---|---|---|---|
| Ful | Wild Type | NP_003997 | ATGCTGTGGTGGGAGGAGGTGGAGGACTGCTACGAGCGGGAGGATG | MLWWEEVEDCYERED |
| Length | protein | | TCCAGAAGAAGACCTTCACTAAGTGGGTGAACGCCCAGTTCAGCAAG | VQKKTFTKWVNAQFS |
| human | (CO | | TTCGGCAAGCAGCACATCGAGAATCTATTCAGCGACCTACAGGACGG | KFGKQHIENLFSDLQD |
| dystrophin | nucleotide | | CCGTCGGCTACTAGACCTTTTAGAGGGTCTGACCGGGCAGAAGCTGC | GRRLLDLLEGLTGQKL |
| | seq-3B) | | CCAAGGAGAAGGGTAGCACCAGGGTCCACGCCCTCAACAACGTGAA | PKEKGSTRVHALNNVN |
| | | | CAAGGCTCTGCGGGTGCTGCAGAACAACAACGTGGACCTTGTGAACA | KALRVLQNNNVDLVNI |
| | | | TCGGCAGCACCGACATCGTGGACGGCAACCACAAGCTGACACTGGGC | GSTDIVDGNHKLTLGLI |
| | | | CTGATCTGGAACATCATCCTGCACTGGCAGGTGAAGAACGTGATGAA | WNIILHWQVKNVMKN |
| | | | GAACATTATGGCCGGCCTGCAACAGACCAACAGCGAGAAGATACTCC | IMAGLQQTNSEKILLS |
| | | | TGAGCTGGGTGCGTCAGAGCACCCGGAACTACCCTCAGGTGAACGTC | WVRQSTRNYPQVNVIN |
| | | | ATCAACTTCACAACCTCATGGTCAGACGGCCTTGCCCTAAACGCCCTG | FTTSWSDGLALNALIH |
| | | | ATCCACTCACACCGGCCCGACCTGTTCGACTGGAACAGCGTGGTGTG | SHRPDLFDWNSVVCQ |
| | | | CCAACAGAGCGCCACACAACGGCTAGAGCACGCCTTCAATATCGCCA | QSATQRLEHAFNIARY |
| | | | GGTACCAGCTGGGCATCGAGAAGCTGCTCGACCCCGAGGATGTGGAC | QLGIEKLLDPEDVDTT |
| | | | ACCACCTACCCCGACAAGAAGAGCATCCTGATGTACATAACCAGCCT | YPDKKSILMYITSLFQV |
| | | | GTTCCAGGTGCTACCCCAGCAGGTCAGCATCGAGGCCATCCAAGAGG | LPQQVSIEAIQEVEMLP |
| | | | TCGAGATGCTCCCACGGCCTCCAAAGGTGACCAAGGAGGAGCACTTC | RPPKVTKEEHFQLHHQ |
| | | | CAGCTACACCACCAGATGCACTACAGCCAGCAGATAACCGTGAGCTT | MHYSQQITVSLAQGYE |
| | | | AGCCCAGGGATACGAGCGTACCAGCAGCCCTAAGCCCCGGTTCAAGA | RTSSPKPRFKSYAYTQ |
| | | | GCTACGCTTACACCCAGGCCGCCTACGTCACTACCAGCGACCCAACTC | AAYVTTSDPTRSPFPSQ |
| | | | GGAGCCCATTCCCCTCACAGCACCTAGAGGCACCCGAGGACAAGAGC | HLEAPEDKSFGSSLME |
| | | | TTCGGTAGCAGCCTGATGGAGTCAGAGGTCAACCTGGACAGGTACCA | SEVNLDRYQTALEEVL |
| | | | GACTGCCCTAGAGGAGGTGCTCTCATGGCTACTGTCAGCTGAGGACA | SWLLSAEDTLQAQGEI |
| | | | CCCTCCAGGCACAGGGAGAGATTAGCAACGACGTCGAGGTGGTGAA | SNDVEVVKDQFHTHE |
| | | | GGACCAGTTCCACACCCACGAGGGCTACATGATGGACCTAACTGCTC | GYMMDLTAHQGRVG |
| | | | ACCAGGGGCGTGTGGGAAACATCCTGCAACTCGGCAGCAAGCTGATC | NILQLGSKLIGTGKLSE |
| | | | GGCACCGGCAAGCTGAGCGAGGATGAGGAGACCGAGGTGCAAGAGC | DEETEVQEQMNLLNSR |
| | | | AGATGAACCTACTGAACAGCCGGTGGGAGTGCCTACGGGTGGCCTCC | WECLRVASMEKQSNL |
| | | | ATGGAGAAACAAAGCAACCTACACAGGGTGCTCATGGATCTGCAGAA | HRVLMDLQNQKLKEL |
| | | | CCAGAAGCTGAAAGAGCTCAATGATTGGCTGACCAAGACTGAGGAGC | NDWLTKTEERTRKME |
| | | | GGACACGTAAGATGGAGGAGGAGCCACTGGGTCCCGACCTGGAGGA | EEPLGPDLEDLKRQVQ |
| | | | CCTAAAGCGGCAGGTGCAACAGCACAAGGTCCTTCAAGAGGATCTGG | QHKVLQEDLEQEQVR |
| | | | AGCAAGAGCAGGTGCGGGTGAACAGCTTAACCCACATGGTCGTCGTG | VNSLTHMVVVVDESS |
| | | | GTGGACGAGTCCAGCGGTGACCACGCTACCGCAGCATTAGAGGAGCA | GDHATAALEEQLKVL |
| | | | GCTAAAAGTGCTGGGTGACAGGTGGGCCAACATCTGCCGGTGGACCG | GDRWANICRWTEDRW |
| | | | AGGATAGGTGGGTGCTGCTGCAAGACATCCTACTGAAGTGGCAGCGG | VLLQDILLKWQRLTEE |
| | | | CTCACAGAGGAGCAGTGCCTATTCAGCGCCTGGCTGAGCGAGAAGGA | QCLFSAWLSEKEDAVN |
| | | | GGATGCCGTGAATAAAATCCACACCACCGGGTTCAAGGACCAGAATG | KIHTTGFKDQNEMLSS |
| | | | AGATGCTGTCAAGCTTACAGAAACTGGCCGTGCTGAAGGCCGATCTG | LQKLAVLKADLEKKK |
| | | | GAGAAGAAGAAGCAAAGCATGGGCAAGCTGTACTCACTGAAGCAAG | QSMGKLYSLKQDLLST |
| | | | ATCTGCTGTCCACCCTTAAGAACAAGAGCGTGACCCAGAAGACCGAG | LKNKSVTQKTEAWLD |
| | | | GCCTGGCTCGACAACTTCGCACGGTGCTGGGACAACCTAGTGCAGAA | NFARCWDNLVQKLEK |
| | | | ACTAGAGAAGAGCACAGCACAGATCAGCCAGGCTGTGACTACAACCC | STAQISQAVTTTQPSLT |
| | | | AACCTAGCCTGACTCAGACTACCGTGATGGAGACCGTCACCACAGTG | QTTVMETVTTVTTREQ |
| | | | ACCACCCGGGAGCAGATCCTTGTGAAGCACGCCCAAGAGGAGCTTCC | ILVKHAQEELPPPPPQK |
| | | | ACCTCCACCTCCTCAGAAGAAGCGTCAGATCACCGTCGACTCAGAGA | KRQITVDSEIRKRLDVD |
| | | | TCAGGAAGCGGCTGGACGTCGACATCACCGAGCTGCACAGCTGGATC | ITELHSWITRSEAVLQS |
| | | | ACCCGGTCAGAGGCTGTGCTGCAAAGCCCCGAGTTTGCCATCTTCCGT | PEFAIFRKEGNFSDLKE |
| | | | AAGGAGGGCAACTTCAGCGATCTAAAGGAGAAGGTCAACGCCATCG | KVNAIEREKAEKFRKL |
| | | | AGCGGGAGAAGGCCGAGAAGTTCCGGAAGTTACAGGATGCCTCACGT | QDASRSAQALVEQMV |
| | | | TCCGCTCAGGCCCTCGTGGAGCAGATGGTCAACGAGGGCGTGAATGC | NEGVNADSIKQASEQL |
| | | | CGATAGCATCAAGCAGGCCAGCGAGCAACTGAATAGCCGTTGGATCG | NSRWIEFCQLLSERLN |
| | | | AGTTTTGCCAACTGCTATCCGAGCGTCTTAACTGGCTAGAGTACCAAA | WLEYQNNIIAFYNQLQ |
| | | | ACAACATCATCGCCTTCTACAACCAACTACAGCAGCTTGAGCAGATG | QLEQMTTTAENWLKIQ |
| | | | ACCACCACCGCTGAGAACTGGCTGAAGATACAGCCAACCACTCCTAG | PTTPSEPTAIKSQLKICK |
| | | | CGAGCCCACAGCCATCAAAAGCCAGCTGAAGATTTGTAAGGATGAGG | DEVNRLSDLQPQIERL |
| | | | TGAACAGGCTAAGCGATCTGCAACCCCAGATCGAGCGGCTCAAGATC | KIQSIALKEKGQGPMFL |
| | | | CAGAGCATCGCATTAAAGGAGAAAGGCCAGGGCCCCATGTTCCTGGA | DADFVAFTNHFKQVFS |
| | | | TGCCGACTTCGTGGCCTTCACCAACCACTTCAAGCAGGTCTTTAGCGA | DVQAREKELQTIFDTLP |
| | | | TGTGCAAGCCCGGGAGAAAGAGCTTCAGACAATCTTCGACACTCTGC | PMRYQETMSAIRTWV |
| | | | CTCCCATGCGGTACCAGGAGACAATGAGCGCCATACGGACCTGGGTC | QQSETKLSIPQLSVTDY |
| | | | CAACAGTCCGAGACCAAGCTGAGCATACCCCAGCTGAGCGTGACCGA | EIMEQRLGELQALQSS |
| | | | CTACGAGATCATGGAGCAGCGGCTGGGTGAGCTGCAGGCCCTTCAGT | LQEQQSGLYYLSTTVK |
| | | | CCAGCCTTCAGGAGCAACAGTCCGGCCTGTACTACCTCAGCACTACC | EMSKKAPSEISRKYQSE |
| | | | GTCAAGGAGATGAGCAAGAAGGCACCCTCCGAGATCAGCCGGAAGT | FEEIEGRWKKLSSQLV |
| | | | ACCAGAGCGAGTTCGAGGAGATTGAGGGCCGGTGGAAGAAGTTATC | EHCQKLEEQMNKLRKI |
| | | | AAGCCAGCTAGTGGAGCACTGCCAAAAGCTGGAGGAGCAGATGAAT | QNHIQTLKKWMAEVD |
| | | | AAACTGCGGAAGATCCAGAATCACATCCAGACTCTGAAGAAGTGGAT | VFLKEEWPALGDSEIL |
| | | | GGCCGAGGTGGACGTGTTCTTAAAGGAGGAGTGGCCCGCCTTAGGCG | KKQLKQCRLLVSDIQTI |
| | | | ACTCCGAGATCTTAAAGAAGCAACTCAAGCAGTGTCGGCTCCTGGTG | QPSLNSVNEGGQKIKN |
| | | | AGCGACATTCAGACCATCCAGCCTAGCCTGAATAGCGTGAACGAGGG | EAEPEFASRLETELKEL |
| | | | TGGCCAGAAAATCAAGAATGAGGCCGAGCCTGAGTTCGCATCCCGGC | NTQWDHMCQQVYAR |
| | | | TGGAGACAGAGTTAAAGGAGCTTAACACCCAGTGGGACCACATGTGT | KEALKGGLEKTVSLQK |
| | | | CAGCAGGTGTACGCACGGAAAGAGGCCCTGAAAGGCGGCCTTGAGA | DLSEMHEWMTQAEEE |
| | | | AGACCGTCAGCCTCCAGAAGGATCTGAGCGAGATGCACGAGTGGATG | YLERDFEYKTPDELQK |
| | | | ACCCAAGCTGAGGAGGAGTACCTGGAGCGTGACTTCGAGTACAAGAC | AVEEMKRAKEEAQQK |
| | | | CCCAGATGAGCTCCAGAAAGCAGTCGAGGAGATGAAACGGGCAAAG | EAKVKLLTESVNSVIA |
| | | | GAGGAGGCTCAGCAGAAGGAGGCCAAGGTCAAGCTTCTGACCGAGA | QAPPVAQEALKKELET |
| | | | GCGTGAACTCCGTGATCGCTCAGGCTCCTCCCGTCGCTCAGGAGGCTC | LTTNYQWLCTRLNGK |
| | | | TCAAGAAGGAGCTAGAGACACTGACCACCAACTACCAGTGGCTATGC | CKTLEEVWACWHELL |
| | | | ACTCGGCTGAACGGCAAGTGCAAGACACTCGAGGAGGTGTGGGCTTG | SYLEKANKWLNEVEF |
| | | | TTGGCACGAGCTGCTGAGCTACCTAGAGAAGGCCAACAAGTGGCTGA | KLKTTENIPGGAEEISE |
| | | | ATGAGGTGGAGTTCAAGCTTAAGACAACCGAGAACATCCCTGGTGGC | VLDSLENLMRHSEDNP |
| | | | GCAGAGGAGATAAGCGAGGTGCTGGACAGCTTAGAGAACCTGATGC | NQIRILAQTLTDGGVM |
| | | | GGCACAGCGAGGACAACCCCAACCAGATCCGGATCCTTGCCCAGACC | DELINEELETFNSRWRE |
| | | | TTAACCGACGGTGGCGTGATGGACGAGCTGATCAACGAGGAGCTTGA | LHEEAVRRQKLLEQSI |
| | | | GACCTTCAACAGCCGTTGGCGTGAGCTTCACGAGGAGGCTGTCCGTC | QSAQETEKSLHLIQESL |
| | | | GGCAAAAGTTACTAGAGCAGAGCATCCAGAGCGCCCAAGAGACCGA | TFIDKQLAAYIADKVD |
| | | | GAAGTCACTGCACCTGATCCAGGAGAGCTTAACCTTCATCGACAAGC | AAQMPQEAQKIQSDLT |
| | | | AACTCGCCGCCTACATCGCCGACAAGGTGGACGCCGCCCAAATGCCA | SHEISLEEMKKHNQGK |
| | | | CAAGAGGCACAGAAAATTCAGAGCGACCTGACCAGCCACGAGATTA | EAAQRVLSQIDVAQKK |
| | | | GCCTTGAGGAGATGAAGAAGCACAACCAGGGCAAAGAGGCCGCCCA | LQDVSMKFRLFQKPAN |
| | | | GCGTGTCCTGAGCCAGATCGACGTGGCACAAAAGAAGCTCCAGGACG | FEQRLQESKMILDEVK |
| | | | TGAGCATGAAGTTCCGGCTGTTCCAGAAGCCCGCCAACTTCGAGCAA | MHLPALETKSVEQEVV |
| | | | CGGCTTCAGGAGAGCAAGATGATCTTAGACGAGGTCAAGATGCACCT | QSQLNHCVNLYKSLSE |
| | | | GCCAGCTCTGGAGACCAAGAGCGTCGAGCAGGAGGTGGTGCAAAGC | VKSEVEMVIKTGRQIV |
| | | | CAACTGAATCACTGTGTGAATCTGTACAAAAGCCTTTCAGAGGTGAA | QKKQTENPKELDERVT |
| | | | GAGCGAGGTGGAGATGGTGATAAAGACCGGCCGGCAGATTGTCCAA | ALKLHYNELGAKVTER |
| | | | AAGAAGCAGACCGAGAACCCCAAGGAGCTTGACGAGCGTGTGACCG | KQQLEKCLKLSRKMR |
| | | | CTCTGAAGCTGCACTACAACGAGCTGGGCGCCAAGGTCACAGAGCGG | KEMNVLTEWLAATDM |
| | | | AAGCAGCAGCTCGAGAAGTGCCTGAAACTGAGCCGGAAGATGCGTA | ELTKRSAVEGMPSNLD |
| | | | AGGAGATGAACGTGTTAACCGAGTGGCTGGCAGCCACCGACATGGAG | SEVAWGKATQKEIEKQ |
| | | | CTAACCAAGCGTTCCGCAGTGGAGGGCATGCCCAGCAACCTGGACTC | KVHLKSITEVGEALKT |
| | | | CGAGGTCGCATGGGGAAAGGCCACCCAGAAGGAGATAGAGAAGCAG | VLGKKETLVEDKLSLL |
| | | | AAGGTGCACCTGAAGAGCATAACCGAGGTCGGCGAGGCTTTAAAGAC | NSNWIAVTSRAEEWLN |
| | | | AGTGCTGGGCAAGAAGGAGACCCTTGTCGAGGATAAGCTGTCCCTGC | LLLEYQKHMETFDQN |
| | | | TGAATAGCAACTGGATCGCTGTGACCAGCAGGGCTGAGGAGTGGCTT | VDHITKWIIQADTLLDE |
| | | | AACCTGCTCCTGGAGTACCAAAAGCACATGGAGACATTCGACCAGAA | SEKKKPQQKEDVLKRL |
| | | | CGTGGACCACATCACCAAATGGATCATCCAGGCCGATACCCTGCTAG | KAELNDIRPKVDSTRD |
| | | | ACGAGTCCGAGAAGAAGAAGCCCCAACAGAAAGAGGATGTCCTGAA | QAANLMANRGDHCRK |
| | | | GCGGTTAAAGGCCGAGTTAAATGACATCCGGCCCAAGGTGGACTCAA | LVEPQISELNHRFAAIS |
| | | | CCCGTGATCAGGCCGCCAACCTGATGGCCAACCGGGGCGATCACTGC | HRIKTGKASIPLKELEQ |
| | | | AGGAAGCTGGTGGAGCCCCAAATTAGCGAGTTAAACCACCGGTTCGC | FNSDIQKLLEPLEAEIQ |
| | | | CGCCATTAGCCACCGGATCAAGACCGGTAAGGCCAGCATTCCCCTTA | QGVNLKEEDFNKDMN |
| | | | AAGAGCTAGAGCAGTTCAACAGCGACATACAGAAACTTCTTGAGCCC | EDNEGTVKELLQRGDN |
| | | | CTAGAGGCCGAGATCCAGCAAGGGGTGAATCTGAAAGAGGAGGACT | LQQRITDERKREEIKIK |
| | | | TCAACAAGGACATGAACGAGGACAACGAGGGCACCGTCAAAGAGTT | QQLLQTKHNALKDLRS |
| | | | ACTCCAGCGGGGCGACAACCTGCAACAGCGGATCACCGATGAGCGTA | QRRKKALEISHQWYQ |
| | | | AGCGTGAGGAGATCAAGATCAAGCAGCAGTTACTACAGACCAAGCA | YKRQADDLLKCLDDIE |
| | | | CAACGCCCTAAAGGACCTGAGGTCACAGCGGCGGAAGAAGGCTCTG | KKLASLPEPRDERKIKE |
| | | | GAGATCAGCCACCAGTGGTACCAGTACAAGCGTCAGGCCGACGACCT | IDRELQKKKEELNAVR |
| | | | CCTGAAGTGCCTGGACGACATCGAGAAGAAGCTAGCAAGCCTGCCTG | RQAEGLSEDGAAMAV |
| | | | AGCCACGTGACGAGCGGAAAATTAAGGAGATCGACCGGGAGCTACA | EPTQIQLSKRWREIESK |
| | | | GAAGAAGAAGGAGGAGCTCAACGCCGTGAGGCGTCAAGCTGAGGGA | FAQFRRLNFAQIHTVR |
| | | | CTGTCCGAGGATGGCGCTGCAATGGCCGTGGAGCCTACCCAGATCCA | EETMMVMTEDMPLEIS |
| | | | ACTGAGCAAGAGGTGGCGGGAGATTGAGAGCAAGTTCGCTCAGTTCC | YVPSTYLTEITHVSQAL |
| | | | GGCGGTTAAACTTCGCCCAGATACACACCGTCCGGGAGGAGACTATG | LEVEQLLNAPDLCAKD |
| | | | ATGGTGATGACTGAGGACATGCCCCTTGAGATCAGCTACGTGCCCAG | FEDLFKQEESLKNIKDS |
| | | | CACCTACCTGACCGAGATCACCCACGTGAGCCAGGCACTACTGGAGG | LQQSSGRIDIIHSKKTA |
| | | | TGGAGCAACTACTGAACGCACCAGACCTGTGCGCCAAGGACTTCGAG | ALQSATPVERVKLQEA |
| | | | GACTTATTTAAGCAGGAGGAGTCACTAAAGAACATCAAGGACTCACT | LSQLDFQWEKVNKMY |
| | | | TCAACAGAGCAGCGGCCGGATCGACATCATCCACAGCAAGAAGACC | KDRQGRFDRSVEKWR |
| | | | GCTGCCCTGCAAAGCGCCACCCCAGTGGAGAGGGTGAAGCTGCAAGA | RFHYDIKIFNQWLTEA |
| | | | GGCACTGTCCCAGCTGGACTTCCAATGGGAGAAAGTCAACAAGATGT | EQFLRKTQIPENWEHA |
| | | | ACAAGGACCGGCAAGGCAGGTTCGACCGGAGCGTGGAGAAGTGGCG | KYKWYLKELQDGIGQ |
| | | | GCGGTTCCACTACGACATCAAGATCTTCAACCAATGGCTAACAGAGG | RQTVVRTLNATGEEIIQ |
| | | | CAGAGCAATTCCTGCGGAAGACCCAGATCCCAGAGAATTGGGAGCAC | QSSKTDASILQEKLGSL |
| | | | GCCAAGTACAAGTGGTACCTAAAAGAGCTGCAAGATGGCATTGGCCA | NLRWQEVCKQLSDRK |
| | | | GAGGCAGACCGTGGTGCGGACCCTTAACGCCACCGGCGAGGAGATTA | KRLEEQKNILSEFQRDL |
| | | | TCCAGCAGTCCAGCAAGACCGATGCCAGCATCCTGCAGGAGAAACTG | NEFVLWLEEADNIASIP |
| | | | GGCTCCCTGAACCTGCGGTGGCAGGAGGTGTGCAAGCAGCTGAGCGA | LEPGKEQQLKEKLEQV |
| | | | CCGGAAGAAGCGGCTCGAGGAGCAGAAGAACATCCTGAGCGAGTTC | KLLVEELPLRQGILKQL |
| | | | CAGCGGGACCTGAACGAGTTCGTGCTGTGGCTAGAGGAGGCAGACAA | NETGGPVLVSAPISPEE |
| | | | CATAGCCAGCATACCTCTGGAGCCCGGCAAAGAGCAGCAGCTTAAGG | QDKLENKLKQTNLQWI |
| | | | AGAAGTTAGAGCAGGTGAAGCTTCTAGTCGAGGAGCTCCCTCTGCGG | KVSRALPEKQGEIEAQI |
| | | | CAAGGCATCCTAAAACAACTGAACGAGACAGGTGGCCCCGTCCTTGT | KDLGQLEKKLEDLEEQ |
| | | | GTCCGCCCCAATTAGCCCTGAGGAGCAAGATAAACTGGAGAACAAAC | LNHLLLWLSPIRNQLEI |
| | | | TTAAGCAGACCAATCTGCAGTGGATCAAGGTGTCAAGGGCCCTGCCA | YNQPNQEGPFDVKETE |
| | | | GAGAAGCAGGGCGAGATAGAGGCCCAAATCAAGGACCTGGGCCAGC | LAVQAKQPDVEEILSKG |
| | | | TAGAGAAGAAGCTTGAGGACCTAGAGGAGCAACTTAACCACCTTCTA | QHLYKEKPATQPVKRK |
| | | | CTGTGGCTGAGCCCCATCCGGAATCAGCTGGAGATATACAACCAACC | LEDLSSEWKAVNRLLQ |
| | | | CAATCAGGAGGGCCCCTTCGACGTCAAAGAGACAGAGATCGCCGTGC | ELRAKQPDLAPGLTTIG |
| | | | AGGCCAAGCAGCCAGATGTCGAGGAGATCCTGAGCAAGGGCCAGCA | ASPTQTVTLVTQPVVT |
| | | | CCTCTACAAAGAGAAACCAGCCACCCAACCAGTGAAGCGGAAACTG | KETAISKLEMPSSLMLE |
| | | | GAGGATCTGTCAAGCGAGTGGAAGGCCGTGAACCGGCTTTTACAGGA | VPALADFNRAWTELTD |
| | | | GCTGCGGGCCAAGCAACCCGATCTTGCTCCCGGCCTGACAACCATAG | WLSLLDQVIKSQRVMV |
| | | | GCGCATCACCCACCCAGACAGTGACCCTGGTGACCCAGCCAGTGGTG | GDLEDINEMIIKQKAT |
| | | | ACAAAAGAGACAGCCATAAGCAAACTGGAGATGCCCAGCAGCCTCA | MQDLEQRRPQLEELIT |
| | | | TGCTGGAGGTCCCCGCTTTAGCCGACTTCAACCGGGCCTGGACCGAGT | AAQNLKNKTSNQEAR |
| | | | TAACCGATTGGCTGTCACTGCTGGACCAGGTGATCAAATCCCAGAGG | TIITDRIERIQNQWDEV |
| | | | GTGATGGTCGGGGATCTCGAGGACATTAACGAGATGATCATCAAGCA | QEHLQNRRQQLNEML |
| | | | GAAGGCCACCATGCAGGACCTTGAGCAACGTCGGCCCCAACTCGAGG | KDSTQWLEAKEEAEQ |
| | | | AGCTGATAACCGCTGCCCAGAACCTGAAGAACAAGACCAGCAATCAA | VLGQARAKLESWKEG |
| | | | GAGGCCCGGACCATCATCACCGACAGGATCGAGCGGATCCAAAACCA | PYTVDAIQKKITETKQL |
| | | | GTGGGACGAGGTCCAGGAGCACCTGCAAAACCGTAGGCAGCAGCTA | AKDLRQWQTNVDVAN |
| | | | AACGAGATGCTGAAGGACAGCACCCAATGGCTGGAGGCTAAAGAGG | DLALKLLRDYSADDTR |
| | | | AGGCTGAGCAAGTCCTAGGCCAAGCCCGGGCAAAGCTGGAGAGCTG | KVHMITENINASWRSI |
| | | | GAAGGAGGGACCCTACACCGTGGACGCCATCCAGAAGAAAATCACA | HKRVSEREAALEETHR |
| | | | GAGACCAAACAGCTCGCCAAGGACCTCCGGCAGTGGCAGACCAACGT | LLQQFPLDLEKFLAWL |
| | | | GGACGTGGCCAACGACCTAGCTTTAAAACTGCTGCGGGACTACAGCG | TEAETTANVLQDATRK |
| | | | CTGACGATACCCGGAAGGTGCACATGATCACTGAGAACATCAATGCC | ERLLEDSKGVKELMKQ |
| | | | AGCTGGCGGAGCATCCACAAGCGGGTGTCAGAGCGGGAGGCTGCTCT | WQDLQGEIEAHTDVY |
| | | | GGAGGAGACTCACCGGCTTCTTCAGCAGTTTCCCCTGGACCTGGAGA | HNLDENSQKILRSLEGS |
| | | | AATTCCTGGCCTGGCTGACTGAGGCCGAGACTACCGCCAACGTGTTA | DDAVLLQRRLDNMNF |
| | | | CAGGACGCCACCCGTAAGGAGCGGCTACTCGAGGACAGCAAGGGCG | KWSELRKKSLNIRSHL |
| | | | TGAAGGAGCTGATGAAGCAGTGGCAAGACCTGCAAGGAGAGATCGA | EASSDQWKRLHLSLQE |
| | | | GGCCCACACCGACGTGTACCACAACCTGGACGAGAACAGCCAAAAG | LLVWLQLKDDELSRQ |
| | | | ATTCTGAGGAGCCTGGAGGGAAGCGACGACGCTGTCTTACTTCAGCG | APIGGDFPAVQKQNDV |
| | | | TCGGCTCGATAATATGAACTTCAAATGGAGCGAGCTGAGGAAGAAGT | HRAFKRELKTKEPVIM |
| | | | CCCTGAACATCCGGAGCCACCTTGAGGCTAGCAGCGACCAGTGGAAA | STLETVRIFLTEQPLEG |
| | | | CGGCTTCACCTGTCCCTGCAGGAGCTTCTAGTGTGGCTGCAGCTGAAA | LEKLYQEPRELPPEERA |
| | | | GACGACGAGCTGAGCAGGCAGGCACCCATTGGTGGAGACTTCCCCGC | QNVTRLLRKQAEEVNT |
| | | | CGTGCAGAAGCAGAACGATGTGCACCGGGCCTTCAAGCGGGAGCTGA | EWEKLNLHSADWQRK |
| | | | AGACAAAGGAGCCCGTGATCATGAGCACACTGGAGACTGTGCGGATC | IDETLERLRELQEATDE |
| | | | TTCCTGACCGAGCAGCCACTGGAGGGTCTGGAGAAGCTTTACCAGGA | LDLKLRQAEVIKGSWQ |
| | | | GCCTCGTGAGCTGCCACCAGAGGAGCGTGCTCAGAACGTGACCCGGC | PVGDLLIDSLQDHLEK |
| | | | TACTGCGGAAGCAGGCCGAGGAGGTGAACACCGAGTGGGAGAAGCT | VKALRGEIAPLKENVS |
| | | | GAACCTTCACAGCGCAGACTGGCAGCGTAAAATCGACGAGACACTTG | HVNDLARQLTTLGIQL |
| | | | AGCGGCTGCAAGAGCTCCAAGAGGCCACTGATGAGCTGGATCTGAAG | SPYNLSTLEDLNTRWK |
| | | | CTTCGTCAAGCAGAGGTCATCAAGGGCTCATGGCAGCCAGTGGGTGA | LLQVAVEDRVRQLHE |
| | | | CCTCCTGATCGATAGCCTGCAGGACCACCTTGAGAAGGTGAAGGCCC | AHRDFGPASQHELSTS |
| | | | TTCGGGGCGAGATCGCACCACTAAAAGAGAACGTGTCCCACGTGAAC | VQGPWERAISPNKVPY |
| | | | GATCTAGCTCGGCAGCTGACCACCCTGGGCATCCAGCTTAGCCCCTAC | YINHETQTTCWDHPK |
| | | | AACCTCAGCACCCTCGAGGACCTTAACACCCGGTGGAAGCTGCTGCA | MTELYQSLADLNNVRF |
| | | | GGTGGCTGTGGAGGACCGTGTGCGGCAATTACACGAGGCCCACCGTG | SAYRTAMKLRRLQKA |
| | | | ACTTTGGGCCCGCTAGCCAGCACTTCTTATCAACCAGCGTGCAGGGTC | LCLDLLSLSAACDALD |
| | | | CCTGGGAGAGGGCCATCTCACCTAACAAGGTGCCCTACTACATCAAC | QHNLKQNDQPMDILQI |
| | | | CACGAGACTCAGACCACCTGCTGGGACCACCCTAAGATGACCGAGTT | INCLTTIYDRLEQEHNN |
| | | | ATACCAGAGCCTGGCCGACCTGAATAACGTGCGGTTCTCCGCCTACC | LVNVPLCVDMCLNWL |
| | | | GGACAGCCATGAAACTGAGGCGGCTACAGAAAGCCCTGTGCCTCGAC | LNVYDTGRTGRIRVLS |
| | | | CTCCTGTCACTCAGCGCTGCCTGTGATGCCCTGGACCAGCACAACCTG | FKTGIISLCKAHLEDKY |
| | | | AAGCAGAATGACCAGCCCATGGACATCCTTCAAATCATCAACTGCCT | RYLFKQVASSTGFCDQ |
| | | | CACCACCATCTACGACCGTCTTGAGCAAGAGCACAACAATCTGGTGA | RRLGLLLHDSIQIPRQL |
| | | | ACGTGCCCCTGTGCGTGGACATGTGCCTGAACTGGCTGCTGAACGTGT | GEVASFGGSNIEPSVRS |
| | | | ACGACACAGGCCGTACCGGCAGGATACGGGTCCTGTCATTTAAGACC | CFQFANNKPEIEAALFL |
| | | | GGCATCATCAGCTTATGCAAGGCCCACCTCGAGGACAAGTACCGGTA | DWMRLEPQSMVWLPV |
| | | | CCTGTTCAAGCAAGTGGCCAGCTCCACTGGCTTCTGCGACCAGAGGC | LHRVAAAETAKHQAK |
| | | | GGCTAGGACTGCTGCTGCACGACAGCATTCAGATTCCCCGGCAACTT | CNICKECPIIGFRYRSLK |
| | | | GGGGAGGTGGCTAGCTTCGGTGGCAGCAACATCGAGCCCTCAGTGCG | HFNYDICQSCFFSGRV |
| | | | GAGCTGCTTCCAGTTCGCCAACAACAAGCCCGAGATTGAGGCCGCCT | AKGHKMHYPMVEYCT |
| | | | TATTTCTGGACTGGATGAGGCTGGAGCCACAGAGCATGGTGTGGCTT | PTTSGEDVRDFAKVLK |
| | | | CCCGTGTTACACCGTGTGGCTGCTGCAGAGACCGCAAAACACCAGGC | NKFRTKRYFAKHPRM |
| | | | CAAGTGCAACATTTGCAAAGAGTGCCCCATCATCGGCTTTCGGTACCG | GYLPVQTVLEGDNME |
| | | | GTCCCTGAAGCACTTCAATTACGATATTTGTCAGAGCTGCTTCTTCTC | TPVTLINFWPVDSAPAS |
| | | | AGGACGGGTCGCCAAGGGACACAAAATGCACTACCCCATGGTGGAGT | SPQLSHDDTHSRIEHYA |
| | | | ACTGTACCCCTACCACCTCCGGAGAGGACGTGCGTGATTTCGCTAAG | SRLAEMENSNGSYLND |
| | | | GTGCTGAAGAATAAGTTCCGTACCAAGCGGTACTTCGCCAAGCACCC | SISPNESIDDEHLLIQHY |
| | | | ACGGATGGGCTACCTGCCCGTGCAGACCGTCCTGGAGGGAGACAACA | CQSLNQDSPLSQPRSPA |
| | | | TGGAGACTCCCGTGACCCTTATCAACTTTTGGCCTGTGGACAGCGCAC | QILISLESEERGELERIL |
| | | | CTGCCTCATCCCCTCAACTGAGCCACGATGACACCCACTCACGGATCG | ADLEEENRNLQAEYDR |
| | | | AGCACTACGCTTCACGGCTCGCCGAGATGGAGAACAGCAACGGCAGC | LKQQHEHKGLSPLPSPP |
| | | | TACCTGAATGACAGCATTAGCCCCAACGAGAGCATCGACGACGAGCA | EMMPTSPQSPRDAELI |
| | | | CCTACTGATCCAGCACTACTGCCAGAGCCTGAACCAGGACAGCCCAC | AEAKLLRQHKGRLEAR |
| | | | TGTCACAGCCTCGTAGCCCCGCACAGATTTTAATCAGCCTGGAGTCCG | MQILEDHNKQLESQLH |
| | | | AGGAGCGTGGTGAGCTGGAGCGGATCCTCGCCGATTTAGAGGAGGAG | RLRQLLEQPQAEAKVN |
| | | | AACCGGAACCTGCAGGCCGAGTACGACCGGCTTAAGCAGCAGCACG | GTTVSSPSTSLQRSDSS |
| | | | AGCACAAAGGACTGTCACCACTTCCCAGCCCTCCCGAGATGATGCCT | QPMLLRVVGSQTSDSM |
| | | | ACCAGCCCTCAATCACCCAGGGATGCCGAGCTGATCGCTGAGGCCAA | GEEDLLSPPQDTSTGLE |
| | | | GCTGCTGCGTCAGCACAAGGGGCGGTTAGAGGCACGGATGCAAATAT | EVMEQLNNSFPSSRGR |
| | | | TAGAGGACCACAACAAACAGTTAGAGTCACAACTGCACAGGCTTCGG | NTPGKPMREDTM (SEQ |
| | | | CAGCTGCTGGAGCAACCACAGGCCGAGGCTAAGGTGAACGGCACAA | ID NO: 16) |
| | | | CCGTGAGCAGCCCAAGCACCTCACTCCAGCGGAGCGACAGCAGCCAG | |
| | | | CCCATGTTACTGCGGGTGGTGGGCTCACAGACCAGCGACAGCATGGG | |
| | | | CGAGGAGGACCTTCTGAGCCCACCCCAAGACACCAGCACCGGTCTGG | |
| | | | AGGAGGTCATGGAGCAACTCAATAACAGCTTCCCTTCCTCAAGGGGC | |
| | | | CGGAATACCCCAGGCAAGCCAATGCGGGAGGACACCATG (SEQ ID NO: | |
| | | | 15) | |
| Micro- | ΔR4-R23 | | ATGGGCAAGCCCATTCCTAATCCTCTGCTGGGCCTCGACAGCACCATG | MLWWEEVEDCYERED |
| dystrophin | (CO) | | CTTTGGTGGGAAGAAGTCGAGGACTGCTACGAGCGCGAGGACGTGCA | VQKKTFTKWVNAQFS |
| | | | GAAGAAAACCTTCACCAAATGGGTCAACGCCCAGTTCAGCAAGTTCG | KFGKQHIENLFSDLQD |
| | | | GCAAGCAGCACATCGAGAACCTGTTCAGCGACCTGCAGGATGGCAGA | GRRLLDLLEGLTGQKL |
| | | | AGGCTGCTGGATCTGCTGGAAGGCCTGACAGGACAGAAGCTGCCCAA | PKEKGSTRVHALNNVN |
| | | | AGAGAAGGGCAGCACAAGAGTGCACGCCCTGAACAACGTGAACAAG | KALRVLQNNNVDLVNI |
| | | | GCCCTGAGAGTGCTGCAGAACAACAACGTGGACCTGGTCAACATCGG | GSTDIVDGNHKLTLGLI |
| | | | CAGCACCGACATCGTGGACGGCAACCACAAACTGACCCTGGGCCTGA | WNIILHWQVKNVMKN |
| | | | TCTGGAACATCATCCTGCACTGGCAAGTGAAGAACGTGATGAAGAAC | IMAGLQQTNSEKILLS |
| | | | ATCATGGCCGGCCTGCAGCAGACCAACAGCGAGAAGATTCTGCTGAG | WVRQSTRNYPQVNVIN |
| | | | CTGGGTCCGACAGAGCACCCGGAATTACCCTCAAGTGAACGTGATCA | FTTSWSDGLALNALIH |
| | | | ACTTCACCACCTCTTGGAGCGACGGACTGGCCCTGAATGCCCTGATCC | SHRPDLFDWNSVVCQ |
| | | | ACAGCCACAGACCTGACCTGTTCGACTGGAACAGCGTCGTGTGTCAG | QSATQRLEHAFNIARY |
| | | | CAGAGCGCCACACAGAGGCTGGAACACGCCTTCAATATCGCCAGATA | QLGIEKLLDPEDVDTT |
| | | | CCAGCTGGGCATCGAGAAACTGCTGGACCCCGAGGATGTGGACACCA | YPDKKSILMYITSLFQV |
| | | | CCTATCCTGACAAGAAATCCATCCTCATGTACATCACCAGCCTGTTCC | LPQQVSIEAIQEVEMLP |
| | | | AGGTGCTGCCCCAGCAGGTTTCCATCGAGGCCATTCAAGAGGTCGAG | RPPKVTKEEHFQLHHQ |
| | | | ATGCTGCCCAGACCTCCTAAAGTGACCAAAGAGGAACACTTCCAGCT | MHYSQQITVSLAQGYE |
| | | | GCACCACCAGATGCACTACTCTCAGCAGATCACCGTGTCTCTGGCCCA | RTSSPKPRFKSYAYTQ |
| | | | GGGCTACGAGAGAACAAGCAGCCCCAAGCCTCGGTTCAAGAGCTACG | AAYVTTSDPTRSPFPSQ |
| | | | CCTATACACAGGCCGCCTACGTGACCACCAGCGATCCTACAAGAAGC | HLEAPEDKSFGSSLME |
| | | | CCATTTCCTAGCCAGCACCTCGAGGCCCCTGAGGATAAGAGCTTTGGC | SEVNLDRYQTALEEVL |
| | | | AGCAGCCTGATGGAAAGCGAAGTGAACCTGGACCGCTACCAGACAG | SWLLSAEDTLQAQGEI |
| | | | CCCTGGAAGAGGTTCTGAGCTGGCTGCTGTCTGCCGAGGATACACTG | SNDVEVVKDQFHTHE |
| | | | CAGGCTCAGGGCGAGATCAGCAACGACGTGGAAGTGGTCAAGGACC | GYMMDLTAHQGRVG |
| | | | AGTTTCACACCCACGAGGGCTACATGATGGACCTGACAGCCCACCAG | NILQLGSKLIGTGKLSE |
| | | | GGCAGAGTGGGCAATATTCTGCAGCTGGGCTCCAAGCTGATCGGCAC | DEETEVQEQMNLLNSR |
| | | | AGGCAAGCTGAGCGAGGACGAAGAGACAGAGGTGCAAGAGCAGATG | WECLRVASMEKQSNL |
| | | | AACCTGCTGAACAGCAGATGGGAGTGTCTGAGAGTGGCCAGCATGGA | HRVLMDLQNQKLKEL |
| | | | AAAGCAGAGCAACCTGCACCGGGTGCTGATGGATCTCCAGAACCAGA | NDWLTKTEERTRKME |
| | | | AGCTGAAAGAGCTGAACGACTGGCTGACCAAGACCGAGGAACGGAC | EEPLGPDLEDLKRQVQ |
| | | | CCGGAAGATGGAAGAGGAACCTCTGGGACCCGACCTGGAAGATCTG | QHKVLQEDLEQEQVR |
| | | | AAAAGACAGGTGCAGCAGCATAAGGTGCTGCAAGAGGACCTCGAGC | VNSLTHMVVVVDESS |
| | | | AAGAGCAAGTGCGCGTGAACAGCCTGACACACATGGTGGTGGTCGTG | GDHATAALEEQLKVL |
| | | | GATGAGAGCAGCGGAGATCATGCCACAGCCGCTCTGGAAGAACAGCT | GDRWANICRWTEDRW |
| | | | GAAGGTGCTGGGAGACAGATGGGCCAATATCTGCCGGTGGACCGAG | VLLQDILLKWQRLTEE |
| | | | GATAGATGGGTGCTGCTCCAGGACATCCTGCTGAAGTGGCAGCGGCT | QCLFSAWLSEKEDAVN |
| | | | GACAGAGGAACAGTGCCTGTTTAGCGCCTGGCTGTCCGAGAAAGAGG | KIHTTGFKDQNEMLSS |
| | | | ACGCCGTCAACAAGATCCACACCACCGGCTTCAAGGATCAGAATGAG | LQKLAVLKADLEKKK |
| | | | ATGCTGAGCAGCCTGCAGAAACTGGCCGTGCTGAAGGCTGACCTGGA | QSMGKLYSLKQDLLST |
| | | | AAAGAAAAAGCAGTCCATGGGCAAGCTGTACTCCCTGAAGCAGGACC | LKNKSVTQKTEAWLD |
| | | | TGCTGAGCACACTGAAGAACAAGAGCGTGACCCAGAAAACCGAGGC | NFARCWDNLVQKLEK |
| | | | CTGGCTGGACAACTTCGCCAGATGCTGGGACAACCTGGTGCAGAAGC | STAQISQAVTTTQPSLT |
| | | | TGGAAAAGTCCACCGCTCAGATCTCTCAGGCCGTGACCACAACACAG | QTTVMETVTTVTTREQ |
| | | | CCTTCTCTGACCCAGACCACCGTGATGGAAACAGTGACCACAGTGAC | ILVKHAQEELPPPPPQK |
| | | | AACCCGCGAGCAGATCCTGGTCAAGCACGCCCAAGAAGAACTGCCTC | KRQITVDTLERLRELQE |
| | | | CTCCACCTCCTCAGAAGAAACGGCAGATCACAGTGGACACTCTGGAA | ATDELDLKLRQAEVIK |
| | | | AGGCTTCAAGAATTGCAAGAGGCCACCGACGAGCTGGACCTGAAGCT | GSWQPVGDLLIDSLQD |
| | | | TAGACAGGCTGAAGTCATCAAAGGCAGCTGGCAGCCTGTGGGCGACC | HLEKVKALRGEIAPLK |
| | | | TCCTGATTGATAGCCTGCAGGACCACCTCGAAAAAGTGAAGGCCCTG | ENVSHVNDLARQLTTL |
| | | | CGGGGAGAGATCGCCCCTCTGAAAGAAAACGTGTCCCACGTGAACGA | GIQLSPYNLSTLEDLNT |
| | | | CCTGGCACGGCAGCTTACAACACTGGGGATTCAGCTGAGCCCCTACA | RWKLLQVAVEDRVRQ |
| | | | ACCTGTCCACACTCGAGGACCTGAATACCCGATGGAAGCTCCTCCAG | LHEAHRDFGPASQHFL |
| | | | GTGGCCGTTGAAGATAGAGTGCGGCAGCTGCACGAAGCCCACAGAG | STSVQGPWERAISPNK |
| | | | ATTTTGGACCAGCCAGCCAGCACTTTCTGAGCACCTCAGTGCAAGGCC | VPYYINHETQTTCWDH |
| | | | CCTGGGAGAGAGCTATCAGCCCTAACAAGGTGCCCTACTACATCAAC | PKMTELYQSLADLNNV |
| | | | CACGAGACACAGACAACCTGTTGGGATCACCCCAAGATGACCGAACT | RFSAYRTAMKLRRLQK |
| | | | GTATCAGTCCCTGGCCGATCTGAACAATGTGCGGTTCAGCGCCTACCG | ALCLDLLSLSAACDAL |
| | | | GACCGCAATGAAGCTGCGGAGACTTCAGAAAGCCCTGTGCCTCGACC | DQHNLKQNDQPMDIL |
| | | | TGCTGTCTCTGAGTGCAGCATGTGATGCCCTGGACCAGCATAATCTGA | QIINCLTTIYDRLEQEH |
| | | | AGCAGAACGACCAGCCTATGGACATCCTCCAGATCATCAACTGCCTG | NNLVNVPLCVDMCLN |
| | | | ACCACTATCTACGACCGGCTCGAACAAGAACACAACAACCTCGTGAA | WLLNVYDTGRTGRIRV |
| | | | CGTGCCCCTGTGCGTGGACATGTGCCTCAATTGGCTGCTGAATGTGTA | LSFKTGIISLCKAHLED |
| | | | CGACACCGGCCGGACCGGCAGAATCAGAGTGCTGTCTTTCAAAACCG | KYRYLFKQVASSTGFC |
| | | | GCATCATCTCCCTGTGCAAAGCCCATCTCGAGGATAAGTACCGCTACC | DQRRLGLLLHDSIQIPR |
| | | | TGTTTAAGCAGGTCGCCAGCAGCACCGGCTTTTGCGATCAAAGAAGG | QLGEVASFGGSNIEPSV |
| | | | CTGGGACTCCTGCTGCACGACAGCATTCAGATCCCAAGACAGCTGGG | RSCFQFANNKPEIEAAL |
| | | | CGAAGTGGCTAGCTTCGGCGGCTCTAATATCGAGCCTAGCGTGCGGA | FLDWMRLEPQSMVWL |
| | | | GCTGCTTCCAGTTCGCCAACAACAAGCCCGAGATTGAGGCAGCCCTC | PVLHRVAAAETAKHQ |
| | | | TTCCTGGATTGGATGCGGCTGGAACCCCAGAGCATGGTTTGGCTGCCA | AKCNICKECPIIGFRYR |
| | | | GTGCTGCATAGAGTGGCCGCTGCCGAAACAGCTAAGCACCAGGCCAA | SLKHFNYDICQSCFFSG |
| | | | GTGCAACATCTGCAAAGAGTGCCCCATCATCGGCTTCCGGTACAGAA | RVAKGHKMHYPMVE |
| | | | GCCTGAAGCACTTCAACTACGATATCTGCCAGTCTTGTTTCTTCTCTGG | YCTPTTSGEDVRDFAK |
| | | | CCGCGTGGCCAAGGGCCACAAGATGCATTACCCCATGGTGGAATACT | VLKNKFRTKRYFAKHP |
| | | | GCACCCCTACCACCTCCGGCGAAGATGTCCGGGATTTTGCCAAGGTG | RMGYLPVQTVLEGDN |
| | | | CTCAAAAACAAGTTCCGCACCAAGCGCTACTTCGCAAAGCACCCCAG | METPVTLINFWPVDSA |
| | | | AATGGGCTACCTGCCTGTGCAGACAGTTCTGGAAGGCGATAATATGG | PASSPQLSHDDTHSRIE |
| | | | AAACCCCTGTGACGCTGATCAATTTCTGGCCCGTGGATAGCGCCCCTG | HYASRLAEMENSNGSY |
| | | | CCAGTTCTCCTCAGCTGTCTCACGATGACACCCACTCCAGAATCGAGC | LNDSISPNESIDDEHLLI |
| | | | ACTACGCCTCCAGACTGGCCGAGATGGAAAACAGCAACGGCAGCTAT | QHYCQSLNQDSPLSQP |
| | | | CTGAACGATAGCATCAGCCCCAACGAGAGCATCGACGACGAACATCT | RSPAQILISLESEERGEL |
| | | | GCTCATCCAGCACTACTGTCAGAGCCTGAACCAGGACTCCCCACTGA | ERILADLEEENRNLQA |
| | | | GCCAGCCTAGAAGCCCAGCACAGATTCTGATTTCCCTGGAATCCGAA | EYDRLKQQHEHKGLSP |
| | | | GAACGGGGCGAGCTGGAAAGAATTCTGGCCGACCTCGAGGAAGAAA | LPSPPEMMPTSPQSPRD |
| | | | ACCGGAACCTGCAGGCAGAGTACGACAGACTGAAACAACAGCACGA | AELIAEAKLLRQHKGR |
| | | | GCACAAGGGACTGAGCCCTTTGCCATCTCCTCCAGAAATGATGCCCA | LEARMQILEDHNKQLE |
| | | | CCTCTCCACAGAGCCCCAGAGATGCAGAGCTGATTGCCGAGGCCAAA | SQLHRLRQLLEQPQAE |
| | | | CTCCTGCGCCAGCACAAAGGCAGACTCGAGGCCAGAATGCAGATTCT | AKVNGTTVSSPSTSLQ |
| | | | CGAAGATCACAACAAACAGCTCGAGTCTCAGCTCCACCGCCTGCGGC | RSDSSQPMLLRVVGSQ |
| | | | AATTGCTGGAACAACCACAGGCAGAGGCTAAAGTGAACGGCACCAC | TSDSMGEEDLLSPPQD |
| | | | AGTGTCTAGCCCCAGCACATCCCTGCAGAGATCCGATAGCTCCCAGC | TSTGLEEVMEQLNNSF |
| | | | CAATGCTCCTGAGAGTCGTGGGCAGCCAGACCAGCGATTCTATGGGC | PSSRGRNTPGKPMRED |
| | | | GAAGAAGATCTGCTTAGCCCTCCTCAGGATACAAGCACCGGCCTCGA | TM (SEQ ID NO: 18) |
| | | | AGAAGTTATGGAACAACTTAACAATAGCTTCCCCAGCAGCAGAGGCC | |
| | | | GCAACACACCTGGCAAGCCCATGCGGGAAGATACCATGtaa (SEQ ID | |
| | | | NO: 17) | |
| Mini- | ΔH2-R19 | | ATGGGCAAGCCCATTCCTAATCCTCTGCTGGGCCTCGACAGCACCATG | MLWWEEVEDCYERED |
| dystrophin | (CO) | | CTTTGGTGGGAAGAAGTCGAGGACTGCTACGAGCGCGAGGACGTGCA | VQKKTFTKWVNAQFS |
| | | | GAAGAAAACCTTCACCAAATGGGTCAACGCCCAGTTCAGCAAGTTCG | KFGKQHIENLFSDLQD |
| | | | GCAAGCAGCACATCGAGAACCTGTTCAGCGACCTGCAGGATGGCAGA | GRRLLDLLEGLTGQKL |
| | | | AGGCTGCTGGATCTGCTGGAAGGCCTGACAGGACAGAAGCTGCCCAA | PKEKGSTRVHALNNVN |
| | | | AGAGAAGGGCAGCACAAGAGTGCACGCCCTGAACAACGTGAACAAG | KALRVLQNNNVDLVNI |
| | | | GCCCTGAGAGTGCTGCAGAACAACAACGTGGACCTGGTCAACATCGG | GSTDIVDGNHKLTLGLI |
| | | | CAGCACCGACATCGTGGACGGCAACCACAAACTGACCCTGGGCCTGA | WNIILHWQVKNVMKN |
| | | | TCTGGAACATCATCCTGCACTGGCAAGTGAAGAACGTGATGAAGAAC | IMAGLQQTNSEKILLS |
| | | | ATCATGGCCGGCCTGCAGCAGACCAACAGCGAGAAGATTCTGCTGAG | WVRQSTRNYPQVNVIN |
| | | | CTGGGTCCGACAGAGCACCCGGAATTACCCTCAAGTGAACGTGATCA | FTTSWSDGLALNALIH |
| | | | ACTTCACCACCTCTTGGAGCGACGGACTGGCCCTGAATGCCCTGATCC | SHRPDLFDWNSVVCQ |
| | | | ACAGCCACAGACCTGACCTGTTCGACTGGAACAGCGTCGTGTGTCAG | QSATQRLEHAFNIARY |
| | | | CAGAGCGCCACACAGAGGCTGGAACACGCCTTCAATATCGCCAGATA | QLGIEKLLDPEDVDTT |
| | | | CCAGCTGGGCATCGAGAAACTGCTGGACCCCGAGGATGTGGACACCA | YPDKKSILMYITSLFQV |
| | | | CCTATCCTGACAAGAAATCCATCCTCATGTACATCACCAGCCTGTTCC | LPQQVSIEAIQEVEMLP |
| | | | AGGTGCTGCCCCAGCAGGTTTCCATCGAGGCCATTCAAGAGGTCGAG | RPPKVTKEEHFQLHHQ |
| | | | ATGCTGCCCAGACCTCCTAAAGTGACCAAAGAGGAACACTTCCAGCT | MHYSQQITVSLAQGYE |
| | | | GCACCACCAGATGCACTACTCTCAGCAGATCACCGTGTCTCTGGCCCA | RTSSPKPRFKSYAYTQ |
| | | | GGGCTACGAGAGAACAAGCAGCCCCAAGCCTCGGTTCAAGAGCTACG | AAYVTTSDPTRSPFPSQ |
| | | | CCTATACACAGGCCGCCTACGTGACCACCAGCGATCCTACAAGAAGC | HLEAPEDKSFGSSLME |
| | | | CCATTTCCTAGCCAGCACCTCGAGGCCCCTGAGGATAAGAGCTTTGGC | SEVNLDRYQTALEEVL |
| | | | AGCAGCCTGATGGAAAGCGAAGTGAACCTGGACCGCTACCAGACAG | SWLLSAEDTLQAQGEI |
| | | | CCCTGGAAGAGGTTCTGAGCTGGCTGCTGTCTGCCGAGGATACACTG | SNDVEVVKDQFHTHE |
| | | | CAGGCTCAGGGCGAGATCAGCAACGACGTGGAAGTGGTCAAGGACC | GYMMDLTAHQGRVG |
| | | | AGTTTCACACCCACGAGGGCTACATGATGGACCTGACAGCCCACCAG | NILQLGSKLIGTGKLSE |
| | | | GGCAGAGTGGGCAATATTCTGCAGCTGGGCTCCAAGCTGATCGGCAC | DEETEVQEQMNLLNSR |
| | | | AGGCAAGCTGAGCGAGGACGAAGAGACAGAGGTGCAAGAGCAGATG | WECLRVASMEKQSNL |
| | | | AACCTGCTGAACAGCAGATGGGAGTGTCTGAGAGTGGCCAGCATGGA | HRVLMDLQNQKLKEL |
| | | | AAAGCAGAGCAACCTGCACCGGGTGCTGATGGATCTCCAGAACCAGA | NDWLTKTEERTRKME |
| | | | AGCTGAAAGAGCTGAACGACTGGCTGACCAAGACCGAGGAACGGAC | EEPLGPDLEDLKRQVQ |
| | | | CCGGAAGATGGAAGAGGAACCTCTGGGACCCGACCTGGAAGATCTG | QHKVLQEDLEQEQVR |
| | | | AAAAGACAGGTGCAGCAGCATAAGGTGCTGCAAGAGGACCTCGAGC | VNSLTHMVVVVDESS |
| | | | AAGAGCAAGTGCGCGTGAACAGCCTGACACACATGGTGGTGGTCGTG | GDHATAALEEQLKVL |
| | | | GATGAGAGCAGCGGAGATCATGCCACAGCCGCTCTGGAAGAACAGCT | GDRWANICRWTEDRW |
| | | | GAAGGTGCTGGGAGACAGATGGGCCAATATCTGCCGGTGGACCGAG | VLLQDILLKWQRLTEE |
| | | | GATAGATGGGTGCTGCTCCAGGACATCCTGCTGAAGTGGCAGCGGCT | QCLFSAWLSEKEDAVN |
| | | | GACAGAGGAACAGTGCCTGTTTAGCGCCTGGCTGTCCGAGAAAGAGG | KIHTTGFKDQNEMLSS |
| | | | ACGCCGTCAACAAGATCCACACCACCGGCTTCAAGGATCAGAATGAG | LQKLAVLKADLEKKK |
| | | | ATGCTGAGCAGCCTGCAGAAACTGGCCGTGCTGAAGGCTGACCTGGA | QSMGKLYSLKQDLLST |
| | | | AAAGAAAAAGCAGTCCATGGGCAAGCTGTACTCCCTGAAGCAGGACC | LKNKSVTQKTEAWLD |
| | | | TGCTGAGCACACTGAAGAACAAGAGCGTGACCCAGAAAACCGAGGC | NFARCWDNLVQKLEK |
| | | | CTGGCTGGACAACTTCGCCAGATGCTGGGACAACCTGGTGCAGAAGC | STAQISQQPDLAPGLTT |
| | | | TGGAAAAGTCCACCGCTCAGATCTCTCAGCAGCCCGATCTGGCCCCTG | IGASPTQTVTLVTQPVV |
| | | | GCCTGACAACAATTGGAGCTAGCCCCACTCAGACCGTGACACTGGTT | TKETAISKLEMPSSLML |
| | | | ACACAGCCAGTGGTCACCAAAGAGACAGCCATCTCCAAACTGGAAAT | EVPALADFNRAWTELT |
| | | | GCCCTCTAGCCTGATGTTGGAGGTGCCCGCACTGGCCGACTTCAATAG | DWLSLLDQVIKSQRVM |
| | | | AGCTTGGACCGAGCTGACTGATTGGCTGTCCCTCCTGGACCAAGTGAT | VGDLEDINEMIIKQKAT |
| | | | CAAGAGCCAGAGAGTGATGGTCGGAGATCTTGAGGACATCAACGAA | MQDLEQRRPQLEELIT |
| | | | ATGATTATCAAGCAGAAAGCCACCATGCAGGATCTCGAACAGCGCAG | AAQNLKNKTSNQEAR |
| | | | ACCCCAGCTTGAAGAACTGATCACAGCCGCACAGAATCTGAAAAACA | TIITDRIERIQNQWDEV |
| | | | AGACCAGCAATCAAGAAGCCAGGACCATCATCACCGACCGGATCGA | QEHLQNRRQQLNEML |
| | | | GCGGATTCAGAACCAGTGGGATGAAGTCCAAGAGCATCTGCAGAACC | KDSTQWLEAKEEAEQ |
| | | | GGCGGCAGCAGCTTAACGAAATGCTGAAGGACAGCACACAGTGGCTC | VLGQARAKLESWKEG |
| | | | GAAGCTAAAGAAGAAGCCGAGCAGGTTCTGGGACAAGCCAGAGCCA | PYTVDAIQKKITETKQL |
| | | | AGCTGGAATCCTGGAAAGAGGGACCCTACACCGTCGACGCCATTCAG | AKDLRQWQTNVDVAN |
| | | | AAAAAAATCACCGAGACAAAACAGCTGGCCAAGGATCTGCGGCAGT | DLALKLLRDYSADDTR |
| | | | GGCAGACCAATGTGGACGTGGCAAATGACCTGGCTCTGAAACTGCTC | KVHMITENINASWRSI |
| | | | CGGGACTACAGCGCCGACGACACCAGAAAGGTGCACATGATCACAG | HKRVSEREAALEETHR |
| | | | AGAACATCAACGCCTCTTGGCGGAGCATCCACAAGCGGGTGTCCGAA | LLQQFPLDLEKFLAWL |
| | | | AGAGAGGCTGCCCTCGAGGAAACACACAGACTGCTGCAACAGTTTCC | TEAETTANVLQDATRK |
| | | | CCTGGACCTTGAGAAATTCCTGGCCTGGCTCACCGAGGCCGAAACCA | ERLLEDSKGVKELMKQ |
| | | | CAGCTAATGTCCTCCAGGATGCCACACGGAAAGAGCGGTTGCTGGAA | WQDLQGEIEAHTDVY |
| | | | GATAGCAAAGGGGTCAAAGAACTTATGAAGCAATGGCAGGACCTCC | HNLDENSQKILRSLEGS |
| | | | AAGGGGAGATCGAGGCTCACACCGACGTGTACCACAATCTGGATGAG | DDAVLLQRRLDNMNF |
| | | | AACAGTCAGAAGATCCTGCGGTCCCTGGAAGGCTCCGATGATGCTGT | KWSELRKKSLNIRSHL |
| | | | GTTGCTCCAGCGGAGACTGGATAACATGAACTTCAAGTGGAGCGAGC | EASSDQWKRLHLSLQE |
| | | | TGCGGAAAAAGTCCCTGAACATCAGAAGCCATCTGGAAGCCAGCAGC | LLVWLQLKDDELSRQ |
| | | | GACCAGTGGAAAAGACTCCACCTGAGCCTCCAAGAGCTTCTCGTGTG | APIGGDFPAVQKQNDV |
| | | | GCTGCAGCTCAAGGACGATGAGCTGAGCAGACAGGCCCCAATCGGCG | HRAFKRELKTKEPVIM |
| | | | GAGATTTTCCCGCCGTGCAGAAACAGAACGACGTGCACAGAGCCTTC | STLETVRIFLTEQPLEG |
| | | | AAGCGGGAACTCAAGACCAAAGAACCCGTGATTATGAGCACCCTGGA | LEKLYQEPRELPPEERA |
| | | | AACTGTGCGGATCTTTCTGACCGAGCAGCCTCTGGAAGGACTCGAGA | QNVTRLLRKQAEEVNT |
| | | | AGCTGTACCAAGAACCTAGAGAGCTGCCTCCTGAGGAACGGGCCCAG | EWEKLNLHSADWQRK |
| | | | AACGTTACACGGCTGCTGAGAAAACAGGCCGAGGAAGTGAATACCG | IDETLERLRELQEATDE |
| | | | AGTGGGAAAAGCTGAATCTCCACTCCGCCGACTGGCAGCGGAAGATC | LDLKLRQAEVIKGSWQ |
| | | | GATGAGACTCTGGAAAGGCTTCAAGAATTGCAAGAGGCCACCGACGA | PVGDLLIDSLQDHLEK |
| | | | GCTGGACCTGAAGCTTAGACAGGCTGAAGTCATCAAAGGCAGCTGGC | VKALRGEIAPLKENVS |
| | | | AGCCTGTGGGCGACCTCCTGATTGATAGCCTGCAGGACCACCTCGAA | HVNDLARQLTTLGIQL |
| | | | AAAGTGAAGGCCCTGCGGGGAGAGATCGCCCCTCTGAAAGAAAACG | SPYNLSTLEDLNTRWK |
| | | | TGTCCCACGTGAACGACCTGGCACGGCAGCTTACAACACTGGGGATT | LLQVAVEDRVRQLHE |
| | | | CAGCTGAGCCCCTACAACCTGTCCACACTCGAGGACCTGAATACCCG | AHRDFGPASQHFLSTS |
| | | | ATGGAAGCTCCTCCAGGTGGCCGTTGAAGATAGAGTGCGGCAGCTGC | VQGPWERAISPNKVPY |
| | | | ACGAAGCCCACAGAGATTTTGGACCAGCCAGCCAGCACTTTCTGAGC | YINHETQTTCWDHPK |
| | | | ACCTCAGTGCAAGGCCCCTGGGAGAGAGCTATCAGCCCTAACAAGGT | MTELYQSLADLNNVRF |
| | | | GCCCTACTACATCAACCACGAGACACAGACAACCTGTTGGGATCACC | SAYRTAMKLRRLQKA |
| | | | CCAAGATGACCGAACTGTATCAGTCCCTGGCCGATCTGAACAATGTG | LCLDLLSLSAACDALD |
| | | | CGGTTCAGCGCCTACCGGACCGCAATGAAGCTGCGGAGACTTCAGAA | QHNLKQNDQPMDILQI |
| | | | AGCCCTGTGCCTCGACCTGCTGTCTCTGAGTGCAGCATGTGATGCCCT | INCLTTIYDRLEQEHNN |
| | | | GGACCAGCATAATCTGAAGCAGAACGACCAGCCTATGGACATCCTCC | LVNVPLCVDMCLNWL |
| | | | AGATCATCAACTGCCTGACCACTATCTACGACCGGCTCGAACAAGAA | LNVYDTGRTGRIRVLS |
| | | | CACAACAACCTCGTGAACGTGCCCCTGTGCGTGGACATGTGCCTCAAT | FKTGIISLCKAHLEDKY |
| | | | TGGCTGCTGAATGTGTACGACACCGGCCGGACCGGCAGAATCAGAGT | RYLFKQVASSTGFCDQ |
| | | | GCTGTCTTTCAAAACCGGCATCATCTCCCTGTGCAAAGCCCATCTCGA | RRLGLLLHDSIQIPRQL |
| | | | GGATAAGTACCGCTACCTGTTTAAGCAGGTCGCCAGCAGCACCGGCT | GEVASFGGSNIEPSVRS |
| | | | TTTGCGATCAAAGAAGGCTGGGACTCCTGCTGCACGACAGCATTCAG | CFQFANNKPEIEAALFL |
| | | | ATCCCAAGACAGCTGGGCGAAGTGGCTAGCTTCGGCGGCTCTAATAT | DWMRLEPQSMVWLPV |
| | | | CGAGCCTAGCGTGCGGAGCTGCTTCCAGTTCGCCAACAACAAGCCCG | LHRVAAAETAKHQAK |
| | | | AGATTGAGGCAGCCCTCTTCCTGGATTGGATGCGGCTGGAACCCCAG | CNICKECPIIGFRYRSLK |
| | | | AGCATGGTTTGGCTGCCAGTGCTGCATAGAGTGGCCGCTGCCGAAAC | HFNYDICQSCFFSGRV |
| | | | AGCTAAGCACCAGGCCAAGTGCAACATCTGCAAAGAGTGCCCCATCA | AKGHKMHYPMVEYCT |
| | | | TCGGCTTCCGGTACAGAAGCCTGAAGCACTTCAACTACGATATCTGCC | PTTSGEDVRDFAKVLK |
| | | | AGTCTTGTTTCTTCTCTGGCCGCGTGGCCAAGGGCCACAAGATGCATT | NKFRTKRYFAKHPRM |
| | | | ACCCCATGGTGGAATACTGCACCCCTACCACCTCCGGCGAAGATGTC | GYLPVQTVLEGDNME |
| | | | CGGGATTTTGCCAAGGTGCTCAAAAACAAGTTCCGCACCAAGCGCTA | TPVTLINFWPVDSAPAS |
| | | | CTTCGCAAAGCACCCCAGAATGGGCTACCTGCCTGTGCAGACAGTTCT | SPQLSHDDTHSRIEHYA |
| | | | GGAAGGCGATAATATGGAAACCCCTGTGACGCTGATCAATTTCTGGC | SRLAEMENSNGSYLND |
| | | | CCGTGGATAGCGCCCCTGCCAGTTCTCCTCAGCTGTCTCACGATGACA | SISPNESIDDEHLLIQHY |
| | | | CCCACTCCAGAATCGAGCACTACGCCTCCAGACTGGCCGAGATGGAA | CQSLNQDSPLSQPRSPA |
| | | | AACAGCAACGGCAGCTATCTGAACGATAGCATCAGCCCCAACGAGAG | QILISLESEERGELERIL |
| | | | CATCGACGACGAACATCTGCTCATCCAGCACTACTGTCAGAGCCTGA | ADLEEENRNLQAEYDR |
| | | | ACCAGGACTCCCCACTGAGCCAGCCTAGAAGCCCAGCACAGATTCTG | LKQQHEHKGLSPLPSPP |
| | | | ATTTCCCTGGAATCCGAAGAACGGGGCGAGCTGGAAAGAATTCTGGC | EMMPTSPQSPRDAELI |
| | | | CGACCTCGAGGAAGAAAACCGGAACCTGCAGGCAGAGTACGACAGA | AEAKLLRQHKGRLEAR |
| | | | CTGAAACAACAGCACGAGCACAAGGGACTGAGCCCTTTGCCATCTCC | MQILEDHNKQLESQLH |
| | | | TCCAGAAATGATGCCCACCTCTCCACAGAGCCCCAGAGATGCAGAGC | RLRQLLEQPQAEAKVN |
| | | | TGATTGCCGAGGCCAAACTCCTGCGCCAGCACAAAGGCAGACTCGAG | GTTVSSPSTSLQRSDSS |
| | | | GCCAGAATGCAGATTCTCGAAGATCACAACAAACAGCTCGAGTCTCA | QPMLLRVVGSQTSDSM |
| | | | GCTCCACCGCCTGCGGCAATTGCTGGAACAACCACAGGCAGAGGCTA | GEEDLLSPPQDTSTGLE |
| | | | AAGTGAACGGCACCACAGTGTCTAGCCCCAGCACATCCCTGCAGAGA | EVMEQLNNSFPSSRGR |
| | | | TCCGATAGCTCCCAGCCAATGCTCCTGAGAGTCGTGGGCAGCCAGAC | NTPGKPMREDTM (SEQ |
| | | | CAGCGATTCTATGGGCGAAGAAGATCTGCTTAGCCCTCCTCAGGATA | ID NO: 20) |
| | | | CAAGCACCGGCCTCGAAGAAGTTATGGAACAACTTAACAATAGCTTC | |
| | | | CCCAGCAGCAGAGGCCGCAACACACCTGGCAAGCCCATGCGGGAAG | |
| | | | ATACCATGtaa (SEQ ID NO: 19) | |
| | Becker | | ATGCTTTGGTGGGAAGAAGTCGAGGACTGCTACGAGCGCGAGGACGT | MLWWEEVEDCYERED |
| | variant | | GCAGAAGAAAACCTTCACCAAATGGGTCAACGCCCAGTTCAGCAAGT | VQKKTFTKWVNAQFS |
| | | | TCGGCAAGCAGCACATCGAGAACCTGTTCAGCGACCTGCAGGATGGC | KFGKQHIENLFSDLQD |
| | | | AGAAGGCTGCTGGATCTGCTGGAAGGCCTGACAGGACAGAAGCTGCC | GRRLLDLLEGLTGQKL |
| | | | CAAAGAGAAGGGCAGCACAAGAGTGCACGCCCTGAACAACGTGAAC | PKEKGSTRVHALNNVN |
| | | | AAGGCCCTGAGAGTGCTGCAGAACAACAACGTGGACCTGGTCAACAT | KALRVLQNNNVDLVNI |
| | | | CGGCAGCACCGACATCGTGGACGGCAACCACAAACTGACCCTGGGCC | GSTDIVDGNHKLTLGLI |
| | | | TGATCTGGAACATCATCCTGCACTGGCAAGTGAAGAACGTGATGAAG | WNIILHWQVKNVMKN |
| | | | AACATCATGGCCGGCCTGCAGCAGACCAACAGCGAGAAGATTCTGCT | IMAGLQQTNSEKILLS |
| | | | GAGCTGGGTCCGACAGAGCACCCGGAATTACCCTCAAGTGAACGTGA | WVRQSTRNYPQVNVIN |
| | | | TCAACTTCACCACCTCTTGGAGCGACGGACTGGCCCTGAATGCCCTGA | FTTSWSDGLALNALIH |
| | | | TCCACAGCCACAGACCTGACCTGTTCGACTGGAACAGCGTCGTGTGTC | SHRPDLFDWNSVVCQ |
| | | | AGCAGAGCGCCACACAGAGGCTGGAACACGCCTTCAATATCGCCAGA | QSATQRLEHAFNIARY |
| | | | TACCAGCTGGGCATCGAGAAACTGCTGGACCCCGAGGATGTGGACAC | QLGIEKLLDPEDVDTT |
| | | | CACCTATCCTGACAAGAAATCCATCCTCATGTACATCACCAGCCTGTT | YPDKKSILMYITSLFQV |
| | | | CCAGGTGCTGCCCCAGCAGGTTTCCATCGAGGCCATTCAAGAGGTCG | LPQQVSIEAIQEVEMLP |
| | | | AGATGCTGCCCAGACCTCCTAAAGTGACCAAAGAGGAACACTTCCAG | RPPKVTKEEHFQLHHQ |
| | | | CTGCACCACCAGATGCACTACTCTCAGCAGATCACCGTGTCTCTGGCC | MHYSQQITVSLAQGYE |
| | | | CAGGGCTACGAGAGAACAAGCAGCCCCAAGCCTCGGTTCAAGAGCTA | RTSSPKPRFKSYAYTQ |
| | | | CGCCTATACACAGGCCGCCTACGTGACCACCAGCGATCCTACAAGAA | AAYVTTSDPTRSPFPSQ |
| | | | GCCCATTTCCTAGCCAGCACCTCGAGGCCCCTGAGGATAAGAGCTTTG | HLEAPEDKSFGSSLME |
| | | | GCAGCAGCCTGATGGAAAGCGAAGTGAACCTGGACCGCTACCAGAC | SEVNLDRYQTALEEVL |
| | | | AGCCCTGGAAGAGGTTCTGAGCTGGCTGCTGTCTGCCGAGGATACAC | SWLLSAEDTLQAQGEI |
| | | | TGCAGGCTCAGGGCGAGATCAGCAACGACGTGGAAGTGGTCAAGGA | SNDVEVVKDQFHTHE |
| | | | CCAGTTTCACACCCACGAGGGCTACATGATGGACCTGACAGCCCACC | GYMMDLTAHQGRVG |
| | | | AGGGCAGAGTGGGCAATATTCTGCAGCTGGGCTCCAAGCTGATCGGC | NILQLGSKLIGTGKLSE |
| | | | ACAGGCAAGCTGAGCGAGGACGAAGAGACAGAGGTGCAAGAGCAGA | DEETEVQEQMNLLNSR |
| | | | TGAACCTGCTGAACAGCAGATGGGAGTGTCTGAGAGTGGCCAGCATG | WECLRVASMEKQSNL |
| | | | GAAAAGCAGAGCAACCTGCACCGGGTGCTGATGGATCTCCAGAACCA | HRVLMDLQNQKLKEL |
| | | | GAAGCTGAAAGAGCTGAACGACTGGCTGACCAAGACCGAGGAACGG | NDWLTKTEERTRKME |
| | | | ACCCGGAAGATGGAAGAGGAACCTCTGGGACCCGACCTGGAAGATCT | EEPLGPDLEDLKRQVQ |
| | | | GAAAAGACAGGTGCAGCAGCATAAGGTGCTGCAAGAGGACCTCGAG | QHKVLQEDLEQEQVR |
| | | | CAAGAGCAAGTGCGCGTGAACAGCCTGACACACATGGTGGTGGTCGT | VNSLTHMVVVVDESS |
| | | | GGATGAGAGCAGCGGAGATCATGCCACAGCCGCTCTGGAAGAACAG | GDHATAALEEQLKVL |
| | | | CTGAAGGTGCTGGGAGACAGATGGGCCAATATCTGCCGGTGGACCGA | GDRWANICRWTEDRW |
| | | | GGATAGATGGGTGCTGCTCCAGGACATCCTGCTGAAGTGGCAGCGGC | VLLQDILLKWQRLTEE |
| | | | TGACAGAGGAACAGTGCCTGTTTAGCGCCTGGCTGTCCGAGAAAGAG | QCLFSAWLSEKEDAVN |
| | | | GACGCCGTCAACAAGATCCACACCACCGGCTTCAAGGATCAGAATGA | KIHTTGFKDQNEMLSS |
| | | | GATGCTGAGCAGCCTGCAGAAACTGGCCGTGCTGAAGGCTGACCTGG | LQKLAVLKADLEKKK |
| | | | AAAAGAAAAAGCAGTCCATGGGCAAGCTGTACTCCCTGAAGCAGGA | QSMGKLYSLKQDLLST |
| | | | CCTGCTGAGCACACTGAAGAACAAGAGCGTGACCCAGAAAACCGAG | LKNKSVTQKTEAWLD |
| | | | GCCTGGCTGGACAACTTCGCCAGATGCTGGGACAACCTGGTGCAGAA | NFARCWDNLVQKLEK |
| | | | GCTGGAAAAGTCCACCGCTCAGGAAACCGAGATTGCCGTGCAGGCCA | STAQETEIAVQAKQPD |
| | | | AGCAGCCAGATGTGGAAGAGATCCTGAGCAAGGGACAGCACCTGTA | VEEILSKGQHLYKEKP |
| | | | CAAAGAGAAACCCGCCACTCAGCCCGTGAAGAGAAAGCTCGAGGAT | ATQPVKRKLEDLSSEW |
| | | | CTGAGCAGCGAGTGGAAGGCCGTGAACAGACTCCTGCAAGAGCTGA | KAVNRLLQELRAKQPD |
| | | | GAGCCAAACAGCCCGATCTGGCCCCTGGCCTGACAACAATTGGAGCT | LAPGLTTIGASPTQTVT |
| | | | AGCCCCACACAGACCGTGACACTGGTTACACAGCCCGTGGTCACCAA | LVTQPVVTKETAISKLE |
| | | | AGAGACAGCCATCAGCAAACTGGAAATGCCCTCTAGCCTGATGCTCG | MPSSLMLEVPALADFN |
| | | | AAGTGCCCGCTCTGGCCGACTTCAATAGAGCCTGGACCGAGCTGACA | RAWTELTDWLSLLDQ |
| | | | GATTGGCTGAGCCTCCTGGACCAAGTGATCAAGAGCCAGAGAGTGAT | VIKSQRVMVGDLEDIN |
| | | | GGTCGGAGATCTCGAGGACATCAACGAGATGATCATTAAGCAGAAAG | EMIIKQKATMQDLEQR |
| | | | CCACCATGCAGGATCTCGAACAGAGAAGGCCCCAGCTCGAAGAACTG | RPQLEELITAAQNLKN |
| | | | ATCACCGCCGCACAGAACCTGAAAAACAAGACCAGCAATCAAGAAG | KTSNQEARTIITDRIERI |
| | | | CCAGGACCATCATCACCGACCGGATCGAGCGGATCCAGAATCAGTGG | QNQWDEVQEHLQNRR |
| | | | GACGAAGTGCAAGAACATCTGCAGAACCGGCGGCAGCAGCTCAACG | QQLNEMLKDSTQWLE |
| | | | AAATGCTGAAGGATAGCACCCAGTGGCTGGAAGCCAAAGAAGAGGC | AKEEAEQVLGQARAK |
| | | | CGAGCAGGTTCTGGGACAAGCCAGAGCCAAGCTGGAATCCTGGAAA | LESWKEGPYTVDAIQK |
| | | | GAGGGACCCTACACCGTCGACGCCATCCAGAAGAAGATCACCGAGAC | KITETKQLAKDLRQWQ |
| | | | AAAGCAGCTGGCCAAGGACCTGAGACAGTGGCAGACCAATGTGGAC | TNVDVANDLALKLLR |
| | | | GTGGCCAACGATCTGGCTCTGAAGCTGCTGAGGGACTACAGCGCCGA | DYSADDTRKVHMITEN |
| | | | CGACACCAGAAAGGTGCACATGATCACAGAGAACATCAACGCCTCTT | INASWRSIHKRVSEREA |
| | | | GGCGGAGCATCCACAAGCGGGTGTCCGAAAGAGAGGCCGCTCTCGA | ALEETHRLLQQFPLDL |
| | | | GGAAACCCACAGACTGCTTCAGCAGTTCCCTCTGGATCTCGAGAAGTT | EKFLAWLTEAETTANV |
| | | | CCTGGCTTGGCTGACCGAGGCCGAAACCACAGCTAACGTTCTGCAGG | LQDATRKERLLEDSKG |
| | | | ACGCCACACGGAAAGAGAGACTGCTCGAGGACTCCAAGGGCGTCAA | VKELMKQWQDLQGEI |
| | | | AGAACTCATGAAGCAGTGGCAGGATCTGCAGGGCGAAATCGAGGCC | EAHTDVYHNLDENSQ |
| | | | CACACCGACGTGTACCACAACCTGGACGAGAACTCCCAGAAGATCCT | KILRSLEGSDDAVLLQ |
| | | | GCGGAGCCTGGAAGGCAGTGATGATGCTGTGCTCCTCCAGCGGAGAC | RRLDNMNFKWSELRK |
| | | | TGGACAACATGAACTTCAAGTGGAGCGAGCTGCGCAAGAAGTCCCTG | KSLNIRSHLEASSDQW |
| | | | AACATCAGAAGCCACCTGGAAGCCTCCAGCGACCAGTGGAAAAGACT | KRLHLSLQELLVWLQL |
| | | | GCATCTGTCCCTGCAAGAACTGCTCGTGTGGCTGCAGCTCAAGGACG | KDDELSRQAPIGGDFP |
| | | | ACGAACTGAGCAGACAGGCCCCTATCGGCGGAGATTTTCCCGCCGTG | AVQKQNDVHRAFKRE |
| | | | CAGAAACAGAACGACGTGCACAGAGCCTTCAAGCGCGAGCTGAAAA | LKTKEPVIMSTLETVRI |
| | | | CAAAAGAACCCGTCATCATGAGCACCCTGGAAACCGTGCGGATCTTC | FLTEQPLEGLEKLYQEP |
| | | | CTGACAGAGCAGCCTCTCGAAGGACTGGAAAAGCTGTACCAAGAGCC | RELPPEERAQNVTRLL |
| | | | TCGCGAGCTGCCTCCTGAAGAAAGGGCCCAGAATGTGACCCGGCTGC | RKQAEEVNTEWEKLN |
| | | | TGAGAAAGCAGGCCGAGGAAGTGAATACCGAGTGGGAGAAACTGAA | LHSADWQRKIDETLER |
| | | | CCTGCACTCCGCCGACTGGCAGCGGAAGATCGATGAGACACTGGAAC | LRELQEATDELDLKLR |
| | | | GGCTGCGGGAACTGCAAGAAGCTACCGACGAGCTGGACCTGAAGCTG | QAEVIKGSWQPVGDLL |
| | | | AGACAGGCCGAAGTCATCAAAGGATCCTGGCAGCCTGTGGGCGACCT | IDSLQDHLEKVKALRG |
| | | | GCTCATCGATTCTCTGCAGGATCACCTCGAGAAAGTGAAGGCCCTGC | EIAPLKENVSHVNDLA |
| | | | GGGGAGAAATCGCCCCTCTGAAAGAAAACGTGTCCCACGTGAACGAC | RQLTTLGIQLSPYNLST |
| | | | CTGGCCAGACAGCTGACAACACTGGGCATCCAGCTGAGCCCCTACAA | LEDLNTRWKLLQVAV |
| | | | CCTGTCCACACTCGAGGACCTGAACACCCGGTGGAAACTGCTCCAAG | EDRVRQLHEAHRDFGP |
| | | | TGGCCGTGGAAGATAGAGTGCGGCAGCTGCACGAAGCCCACAGAGA | ASQHFLSTSVQGPWER |
| | | | TTTTGGACCAGCCAGCCAGCACTTCCTGAGCACCTCAGTTCAAGGCCC | AISPNKVPYYINHETQT |
| | | | CTGGGAGAGAGCTATCAGCCCTAACAAGGTGCCCTACTACATCAACC | TCWDHPKMTELYQSL |
| | | | ACGAGACACAGACCACCTGTTGGGATCACCCCAAGATGACCGAACTG | ADLNNVRFSAYRTAM |
| | | | TATCAGTCCCTGGCCGATCTGAACAATGTGCGGTTCAGCGCCTACCGG | KLRRLQKALCLDLLSL |
| | | | ACCGCTATGAAGCTGCGAAGGCTGCAGAAAGCTCTGTGCCTGGACCT | SAACDALDQHNLKQN |
| | | | GCTCTCTCTGAGCGCTGCTTGTGATGCCCTGGACCAGCATAATCTGAA | DQPMDILQIINCLTTIY |
| | | | GCAGAACGACCAGCCTATGGACATCCTCCAGATCATCAATTGCCTGA | DRLEQEHNNLVNVPLC |
| | | | CCACCATCTACGACCGCCTGGAACAAGAACACAACAACCTCGTGAAC | VDMCLNWLLNVYDTG |
| | | | GTGCCCCTGTGCGTGGACATGTGCCTGAATTGGCTGCTGAACGTGTAC | RTGRIRVLSFKTGIISLC |
| | | | GACACCGGCCGGACAGGCAGAATCAGAGTGCTGTCCTTCAAGACCGG | KAHLEDKYRYLFKQV |
| | | | CATCATCTCCCTGTGCAAGGCCCACCTTGAGGACAAGTACCGCTACCT | ASSTGFCDQRRLGLLL |
| | | | GTTCAAACAGGTGGCCTCCAGCACCGGCTTTTGCGACCAAAGAAGGC | HDSIQIPRQLGEVASFG |
| | | | TGGGACTGCTCCTGCACGACAGCATTCAGATCCCTAGACAGCTGGGC | GSNIEPSVRSCFQFANN |
| | | | GAAGTGGCCTCTTTCGGCGGCTCTAATATCGAGCCTAGCGTGCGGAG | KPEIEAALFLDWMRLE |
| | | | CTGCTTCCAGTTCGCCAACAACAAGCCCGAGATCGAGGCTGCCCTGTT | PQSMVWLPVLHRVAA |
| | | | CCTGGACTGGATGAGACTGGAACCCCAGAGCATGGTCTGGCTGCCTG | AETAKHQAKCNICKEC |
| | | | TGCTGCATAGAGTGGCCGCTGCCGAAACAGCCAAGCACCAGGCCAAG | PIIGFRYRSLKHFNYDIC |
| | | | TGCAACATCTGTAAAGAGTGCCCCATCATCGGCTTCCGGTACAGAAG | QSCFFSGRVAKGHKM |
| | | | CCTGAAGCACTTCAACTACGATATCTGCCAGAGCTGTTTCTTCAGCGG | HYPMVEYCTPTTSGED |
| | | | CAGAGTCGCCAAGGGCCACAAAATGCACTACCCCATGGTGGAATACT | VRDFAKVLKNKFRTKR |
| | | | GCACCCCTACCACCTCCGGCGAAGATGTGCGGGATTTTGCCAAGGTG | YFAKHPRMGYLPVQT |
| | | | CTCAAAAACAAGTTCCGGACCAAGCGCTACTTCGCTAAGCACCCCAG | VLEGDNMETPVTLINF |
| | | | AATGGGCTATCTGCCCGTGCAGACAGTGCTCGAGGGCGACAATATGG | WPVDSAPASSPQLSHD |
| | | | AAACCCCTGTGACACTGATCAATTTCTGGCCCGTGGACAGCGCCCCTG | DTHSRIEHYASRLAEM |
| | | | CTAGTTCTCCTCAACTGAGCCACGACGATACCCACAGCAGGATCGAG | ENSNGSYLNDSISPNESI |
| | | | CACTACGCCAGCAGACTGGCCGAGATGGAAAACAGCAACGGCAGCT | DDEHLLIQHYCQSLNQ |
| | | | ACCTGAACGATAGCATCAGCCCCAACGAGAGCATCGACGACGAGCAT | DSPLSQPRSPAQILISLE |
| | | | CTGCTGATCCAGCACTACTGTCAGAGCCTGAACCAGGACTCCCCACTG | SEERGELERILADLEEE |
| | | | AGCCAGCCTAGAAGCCCTGCACAGATCCTGATCAGCCTGGAATCCGA | NRNLQAEYDRLKQQH |
| | | | GGAAAGGGGCGAGCTGGAAAGAATCCTGGCCGACCTCGAAGAGGAA | EHKGLSPLPSPPEMMP |
| | | | AACCGGAACCTGCAGGCAGAGTACGACAGACTGAAGCAGCAGCACG | TSPQSPRDAELIAEAKL |
| | | | AGCACAAGGGACTGTCTCCACTGCCATCTCCACCTGAAATGATGCCC | LRQHKGRLEARMQILE |
| | | | ACCTCTCCACAGAGCCCCAGAGATGCCGAGCTGATTGCCGAAGCCAA | DHNKQLESQLHRLRQL |
| | | | ACTCCTCAGACAGCACAAAGGCAGACTGGAAGCCCGGATGCAGATTC | LEQPQAEAKVNGTTVS |
| | | | TTGAGGACCACAACAAACAGCTCGAGTCTCAGCTGCACAGACTGAGA | SPSTSLQRSDSSQPMLL |
| | | | CAGCTGCTTGAGCAACCTCAGGCTGAGGCCAAAGTGAACGGCACCAC | RVVGSQTSDSMGEEDL |
| | | | AGTGTCTAGCCCCTCCACTAGTCTGCAGCGGAGCGATTCTAGCCAGCC | LSPPQDTSTGLEEVME |
| | | | AATGCTCCTGAGAGTCGTGGGCTCTCAGACCAGCGATAGCATGGGCG | QLNNSFPSSRGRNTPG |
| | | | AAGAGGATCTGCTCAGCCCTCCTCAGGATACAAGCACCGGCCTGGAA | KPMREDTM (SEQ ID |
| | | | GAAGTTATGGAACAGCTCAACAACAGCTTCCCCAGCAGCAGAGGCAG | NO: 32) |
| | | | AAACACCCCTGGCAAGCCCATGCGCGAAGATACCATG (SEQ ID NO: 31) | |
| Mini- | Becker | | ATGCTTTGGTGGGAAGAAGTCGAGGACTGCTACGAGCGCGAGGACGT | MLWWEEVEDCYERED |
| dystrophin | variant | | GCAGAAGAAAACCTTCACCAAATGGGTCAACGCCCAGTTCAGCAAGT | VQKKTFTKWVNAQFS |
| | | | TCGGCAAGCAGCACATCGAGAACCTGTTCAGCGACCTGCAGGATGGC | KFGKQHIENLFSDLQD |
| | | | AGAAGGCTGCTGGATCTGCTGGAAGGCCTGACAGGACAGAAGCTGCC | GRRLLDLLEGLTGQKL |
| | | | CAAAGAGAAGGGCAGCACAAGAGTGCACGCCCTGAACAACGTGAAC | PKEKGSTRVHALNNVN |
| | | | AAGGCCCTGAGAGTGCTGCAGAACAACAACGTGGACCTGGTCAACAT | KALRVLQNNNVDLVNI |
| | | | CGGCAGCACCGACATCGTGGACGGCAACCACAAACTGACCCTGGGCC | GSTDIVDGNHKLTLGLI |
| | | | TGATCTGGAACATCATCCTGCACTGGCAAGTGAAGAACGTGATGAAG | WNIILHWQVKNVMKN |
| | | | AACATCATGGCCGGCCTGCAGCAGACCAACAGCGAGAAGATTCTGCT | IMAGLQQTNSEKILLS |
| | | | GAGCTGGGTCCGACAGAGCACCCGGAATTACCCTCAAGTGAACGTGA | WVRQSTRNYPQVNVIN |
| | | | TCAACTTCACCACCTCTTGGAGCGACGGACTGGCCCTGAATGCCCTGA | FTTSWSDGLALNALIH |
| | | | TCCACAGCCACAGACCTGACCTGTTCGACTGGAACAGCGTCGTGTGTC | SHRPDLFDWNSVVCQ |
| | | | AGCAGAGCGCCACACAGAGGCTGGAACACGCCTTCAATATCGCCAGA | QSATQRLEHAFNIARY |
| | | | TACCAGCTGGGCATCGAGAAACTGCTGGACCCCGAGGATGTGGACAC | QLGIEKLLDPEDVDTT |
| | | | CACCTATCCTGACAAGAAATCCATCCTCATGTACATCACCAGCCTGTT | YPDKKSILMYITSLFQV |
| | | | CCAGGTGCTGCCCCAGCAGGTTTCCATCGAGGCCATTCAAGAGGTCG | LPQQVSIEAIQEVEMLP |
| | | | AGATGCTGCCCAGACCTCCTAAAGTGACCAAAGAGGAACACTTCCAG | RPPKVTKEEHFQLHHQ |
| | | | CTGCACCACCAGATGCACTACTCTCAGCAGATCACCGTGTCTCTGGCC | MHYSQQITVSLAQGYE |
| | | | CAGGGCTACGAGAGAACAAGCAGCCCCAAGCCTCGGTTCAAGAGCTA | RTSSPKPRFKSYAYTQ |
| | | | CGCCTATACACAGGCCGCCTACGTGACCACCAGCGATCCTACAAGAA | AAYVTTSDPTRSPFPSQ |
| | | | GCCCATTTCCTAGCCAGCACCTCGAGGCCCCTGAGGATAAGAGCTTTG | HLEAPEDKSFGSSLME |
| | | | GCAGCAGCCTGATGGAAAGCGAAGTGAACCTGGACCGCTACCAGAC | SEVNLDRYQTALEEVL |
| | | | AGCCCTGGAAGAGGTTCTGAGCTGGCTGCTGTCTGCCGAGGATACAC | SWLLSAEDTLQAQGEI |
| | | | TGCAGGCTCAGGGCGAGATCAGCAACGACGTGGAAGTGGTCAAGGA | SNDVEVVKDQFHTHE |
| | | | CCAGTTTCACACCCACGAGGGCTACATGATGGACCTGACAGCCCACC | GYMMDLTAHQGRVG |
| | | | AGGGCAGAGTGGGCAATATTCTGCAGCTGGGCTCCAAGCTGATCGGC | NILQLGSKLIGTGKLSE |
| | | | ACAGGCAAGCTGAGCGAGGACGAAGAGACAGAGGTGCAAGAGCAGA | DEETEVQEQMNLLNSR |
| | | | TGAACCTGCTGAACAGCAGATGGGAGTGTCTGAGAGTGGCCAGCATG | WECLRVASMEKQSNL |
| | | | GAAAAGCAGAGCAACCTGCACCGGGTGCTGATGGATCTCCAGAACCA | HRVLMDLQNQKLKEL |
| | | | GAAGCTGAAAGAGCTGAACGACTGGCTGACCAAGACCGAGGAACGG | NDWLTKTEERTRKME |
| | | | ACCCGGAAGATGGAAGAGGAACCTCTGGGACCCGACCTGGAAGATCT | EEPLGPDLEDLKRQVQ |
| | | | GAAAAGACAGGTGCAGCAGCATAAGGTGCTGCAAGAGGACCTCGAG | QHKVLQEDLEQEQVR |
| | | | CAAGAGCAAGTGCGCGTGAACAGCCTGACACACATGGTGGTGGTCGT | VNSLTHMVVVVDESS |
| | | | GGATGAGAGCAGCGGAGATCATGCCACAGCCGCTCTGGAAGAACAG | GDHATAALEEQLKVL |
| | | | CTGAAGGTGCTGGGAGACAGATGGGCCAATATCTGCCGGTGGACCGA | GDRWANICRWTEDRW |
| | | | GGATAGATGGGTGCTGCTCCAGGACATCCTGCTGAAGTGGCAGCGGC | VLLQDILLKWQRLTEE |
| | | | TGACAGAGGAACAGTGCCTGTTTAGCGCCTGGCTGTCCGAGAAAGAG | QCLFSAWLSEKEDAVN |
| | | | GACGCCGTCAACAAGATCCACACCACCGGCTTCAAGGATCAGAATGA | KIHTTGFKDQNEMLSS |
| | | | GATGCTGAGCAGCCTGCAGAAACTGGCCGTGCTGAAGGCTGACCTGG | LQKLAVLKADLEKKK |
| | | | AAAAGAAAAAGCAGTCCATGGGCAAGCTGTACTCCCTGAAGCAGGA | QSMGKLYSLKQDLLST |
| | | | CCTGCTGAGCACACTGAAGAACAAGAGCGTGACCCAGAAAACCGAG | LKNKSVTQKTEAWLD |
| | | | GCCTGGCTGGACAACTTCGCCAGATGCTGGGACAACCTGGTGCAGAA | NFARCWDNLVQKLEK |
| | | | GCTGGAAAAGTCCACCGCTCAGATCAGCCAGATCTACAACCAGCCTA | STAQISQIYNQPNQEGP |
| | | | ATCAAGAGGGCCCCTTCGACGTGAAAGAAACCGAGATTGCCGTGCAG | FDVKETEIAVQAKQPD |
| | | | GCCAAGCAGCCAGATGTGGAAGAGATCCTGAGCAAGGGACAGCACC | VEEILSKGQHLYKEKP |
| | | | TGTACAAAGAGAAACCCGCCACTCAGCCCGTGAAGAGAAAGCTCGA | ATQPVKRKLEDLSSEW |
| | | | GGATCTGAGCAGCGAGTGGAAGGCCGTGAACAGACTCCTGCAAGAG | KAVNRLLQELRAKQPD |
| | | | CTGAGAGCCAAACAGCCCGATCTGGCCCCTGGCCTGACAACAATTGG | LAPGLTTIGASPTQTVT |
| | | | AGCTAGCCCCACACAGACCGTGACACTGGTTACACAGCCCGTGGTCA | LVTQPVVTKETAISKLE |
| | | | CCAAAGAGACAGCCATCAGCAAACTGGAAATGCCCTCTAGCCTGATG | MPSSLMLEVPALADFN |
| | | | CTCGAAGTGCCCGCTCTGGCCGACTTCAATAGAGCCTGGACCGAGCT | RAWTELTDWLSLLDQ |
| | | | GACAGATTGGCTGAGCCTCCTGGACCAAGTGATCAAGAGCCAGAGAG | VIKSQRVMVGDLEDIN |
| | | | TGATGGTCGGAGATCTCGAGGACATCAACGAGATGATCATTAAGCAG | EMIIKQKATMQDLEQR |
| | | | AAAGCCACCATGCAGGATCTCGAACAGAGAAGGCCCCAGCTCGAAG | RPQLEELITAAQNLKN |
| | | | AACTGATCACCGCCGCACAGAACCTGAAAAACAAGACCAGCAATCA | KTSNQEARTIITDRIERI |
| | | | AGAAGCCAGGACCATCATCACCGACCGGATCGAGCGGATCCAGAATC | QNQWDEVQEHLQNRR |
| | | | AGTGGGACGAAGTGCAAGAACATCTGCAGAACCGGCGGCAGCAGCT | QQLNEMLKDSTQWLE |
| | | | CAACGAAATGCTGAAGGATAGCACCCAGTGGCTGGAAGCCAAAGAA | AKEEAEQVLGQARAK |
| | | | GAGGCCGAGCAGGTTCTGGGACAAGCCAGAGCCAAGCTGGAATCCTG | LESWKEGPYTVDAIQK |
| | | | GAAAGAGGGACCCTACACCGTCGACGCCATCCAGAAGAAGATCACC | KITETKQLAKDLRQWQ |
| | | | GAGACAAAGCAGCTGGCCAAGGACCTGAGACAGTGGCAGACCAATG | TNVDVANDLALKLLR |
| | | | TGGACGTGGCCAACGATCTGGCTCTGAAGCTGCTGAGGGACTACAGC | DYSADDTRKVHMITEN |
| | | | GCCGACGACACCAGAAAGGTGCACATGATCACAGAGAACATCAACG | INASWRSIHKRVSEREA |
| | | | CCTCTTGGCGGAGCATCCACAAGCGGGTGTCCGAAAGAGAGGCCGCT | ALEETHRLLQQFPLDL |
| | | | CTCGAGGAAACCCACAGACTGCTTCAGCAGTTCCCTCTGGATCTCGAG | EKFLAWLTEAETTANV |
| | | | AAGTTCCTGGCTTGGCTGACCGAGGCCGAAACCACAGCTAACGTTCT | LQDATRKERLLEDSKG |
| | | | GCAGGACGCCACACGGAAAGAGAGACTGCTCGAGGACTCCAAGGGC | VKELMKQWQDLQGEI |
| | | | GTCAAAGAACTCATGAAGCAGTGGCAGGATCTGCAGGGCGAAATCG | EAHTDVYHNLDENSQ |
| | | | AGGCCCACACCGACGTGTACCACAACCTGGACGAGAACTCCCAGAAG | KILRSLEGSDDAVLLQ |
| | | | ATCCTGCGGAGCCTGGAAGGCAGTGATGATGCTGTGCTCCTCCAGCG | RRLDNMNFKWSELRK |
| | | | GAGACTGGACAACATGAACTTCAAGTGGAGCGAGCTGCGCAAGAAG | KSLNIRSHLEASSDQW |
| | | | TCCCTGAACATCAGAAGCCACCTGGAAGCCTCCAGCGACCAGTGGAA | KRLHLSLQELLVWLQL |
| | | | AAGACTGCATCTGTCCCTGCAAGAACTGCTCGTGTGGCTGCAGCTCAA | KDDELSRQAPIGGDFP |
| | | | GGACGACGAACTGAGCAGACAGGCCCCTATCGGCGGAGATTTTCCCG | AVQKQNDVHRAFKRE |
| | | | CCGTGCAGAAACAGAACGACGTGCACAGAGCCTTCAAGCGCGAGCTG | LKTKEPVIMSTLETVRI |
| | | | AAAACAAAAGAACCCGTCATCATGAGCACCCTGGAAACCGTGCGGAT | FLTEQPLEGLEKLYQEP |
| | | | CTTCCTGACAGAGCAGCCTCTCGAAGGACTGGAAAAGCTGTACCAAG | RELPPEERAQNVTRLL |
| | | | AGCCTCGCGAGCTGCCTCCTGAAGAAAGGGCCCAGAATGTGACCCGG | RKQAEEVNTEWEKLN |
| | | | CTGCTGAGAAAGCAGGCCGAGGAAGTGAATACCGAGTGGGAGAAAC | LHSADWQRKIDETLER |
| | | | TGAACCTGCACTCCGCCGACTGGCAGCGGAAGATCGATGAGACACTG | LRELQEATDELDLKLR |
| | | | GAACGGCTGCGGGAACTGCAAGAAGCTACCGACGAGCTGGACCTGA | QAEVIKGSWQPVGDLL |
| | | | AGCTGAGACAGGCCGAAGTCATCAAAGGATCCTGGCAGCCTGTGGGC | IDSLQDHLEKVKALRG |
| | | | GACCTGCTCATCGATTCTCTGCAGGATCACCTCGAGAAAGTGAAGGC | EIAPLKENVSHVNDLA |
| | | | CCTGCGGGGAGAAATCGCCCCTCTGAAAGAAAACGTGTCCCACGTGA | RQLTTLGIQLSPYNLST |
| | | | ACGACCTGGCCAGACAGCTGACAACACTGGGCATCCAGCTGAGCCCC | LEDLNTRWKLLQVAV |
| | | | TACAACCTGTCCACACTCGAGGACCTGAACACCCGGTGGAAACTGCT | EDRVRQLHEAHRDFGP |
| | | | CCAAGTGGCCGTGGAAGATAGAGTGCGGCAGCTGCACGAAGCCCAC | ASQHELSTSVQGPWER |
| | | | AGAGATTTTGGACCAGCCAGCCAGCACTTCCTGAGCACCTCAGTTCA | AISPNKVPYYINHETQT |
| | | | AGGCCCCTGGGAGAGAGCTATCAGCCCTAACAAGGTGCCCTACTACA | TCWDHPKMTELYQSL |
| | | | TCAACCACGAGACACAGACCACCTGTTGGGATCACCCCAAGATGACC | ADLNNVRFSAYRTAM |
| | | | GAACTGTATCAGTCCCTGGCCGATCTGAACAATGTGCGGTTCAGCGCC | KLRRLQKALCLDLLSL |
| | | | TACCGGACCGCTATGAAGCTGCGAAGGCTGCAGAAAGCTCTGTGCCT | SAACDALDQHNLKQN |
| | | | GGACCTGCTCTCTCTGAGCGCTGCTTGTGATGCCCTGGACCAGCATAA | DQPMDILQIINCLTTIY |
| | | | TCTGAAGCAGAACGACCAGCCTATGGACATCCTCCAGATCATCAATT | DRLEQEHNNLVNVPLC |
| | | | GCCTGACCACCATCTACGACCGCCTGGAACAAGAACACAACAACCTC | VDMCLNWLLNVYDTG |
| | | | GTGAACGTGCCCCTGTGCGTGGACATGTGCCTGAATTGGCTGCTGAAC | RTGRIRVLSFKTGIISLC |
| | | | GTGTACGACACCGGCCGGACAGGCAGAATCAGAGTGCTGTCCTTCAA | KAHLEDKYRYLFKQV |
| | | | GACCGGCATCATCTCCCTGTGCAAGGCCCACCTTGAGGACAAGTACC | ASSTGFCDQRRLGLLL |
| | | | GCTACCTGTTCAAACAGGTGGCCTCCAGCACCGGCTTTTGCGACCAAA | HDSIQIPRQLGEVASFG |
| | | | GAAGGCTGGGACTGCTCCTGCACGACAGCATTCAGATCCCTAGACAG | GSNIEPSVRSCFQFANN |
| | | | CTGGGCGAAGTGGCCTCTTTCGGCGGCTCTAATATCGAGCCTAGCGTG | KPEIEAALFLDWMRLE |
| | | | CGGAGCTGCTTCCAGTTCGCCAACAACAAGCCCGAGATCGAGGCTGC | PQSMVWLPVLHRVAA |
| | | | CCTGTTCCTGGACTGGATGAGACTGGAACCCCAGAGCATGGTCTGGC | AETAKHQAKCNICKEC |
| | | | TGCCTGTGCTGCATAGAGTGGCCGCTGCCGAAACAGCCAAGCACCAG | PIIGFRYRSLKHFNYDIC |
| | | | GCCAAGTGCAACATCTGTAAAGAGTGCCCCATCATCGGCTTCCGGTA | QSCFFSGRVAKGHKM |
| | | | CAGAAGCCTGAAGCACTTCAACTACGATATCTGCCAGAGCTGTTTCTT | HYPMVEYCTPTTSGED |
| | | | CAGCGGCAGAGTCGCCAAGGGCCACAAAATGCACTACCCCATGGTGG | VRDFAKVLKNKFRTKR |
| | | | AATACTGCACCCCTACCACCTCCGGCGAAGATGTGCGGGATTTTGCCA | YFAKHPRMGYLPVQT |
| | | | AGGTGCTCAAAAACAAGTTCCGGACCAAGCGCTACTTCGCTAAGCAC | VLEGDNMETPVTLINF |
| | | | CCCAGAATGGGCTATCTGCCCGTGCAGACAGTGCTCGAGGGCGACAA | WPVDSAPASSPQLSHD |
| | | | TATGGAAACCCCTGTGACACTGATCAATTTCTGGCCCGTGGACAGCGC | DTHSRIEHYASRLAEM |
| | | | CCCTGCTAGTTCTCCTCAACTGAGCCACGACGATACCCACAGCAGGAT | ENSNGSYLNDSISPNESI |
| | | | CGAGCACTACGCCAGCAGACTGGCCGAGATGGAAAACAGCAACGGC | DDEHLLIQHYCQSLNQ |
| | | | AGCTACCTGAACGATAGCATCAGCCCCAACGAGAGCATCGACGACGA | DSPLSQPRSPAQILISLE |
| | | | GCATCTGCTGATCCAGCACTACTGTCAGAGCCTGAACCAGGACTCCCC | SEERGELERILADLEEE |
| | | | ACTGAGCCAGCCTAGAAGCCCTGCACAGATCCTGATCAGCCTGGAAT | NRNLQAEYDRLKQQH |
| | | | CCGAGGAAAGGGGCGAGCTGGAAAGAATCCTGGCCGACCTCGAAGA | EHKGLSPLPSPPEMMP |
| | | | GGAAAACCGGAACCTGCAGGCAGAGTACGACAGACTGAAGCAGCAG | TSPQSPRDAELIAEAKL |
| | | | CACGAGCACAAGGGACTGTCTCCACTGCCATCTCCACCTGAAATGAT | LRQHKGRLEARMQILE |
| | | | GCCCACCTCTCCACAGAGCCCCAGAGATGCCGAGCTGATTGCCGAAG | DHNKQLESQLHRLRQL |
| | | | CCAAACTCCTCAGACAGCACAAAGGCAGACTGGAAGCCCGGATGCA | LEQPQAEAKVNGTTVS |
| | | | GATTCTTGAGGACCACAACAAACAGCTCGAGTCTCAGCTGCACAGAC | SPSTSLQRSDSSQPMLL |
| | | | TGAGACAGCTGCTTGAGCAACCTCAGGCTGAGGCCAAAGTGAACGGC | RVVGSQTSDSMGEEDL |
| | | | ACCACAGTGTCTAGCCCCTCCACTAGTCTGCAGCGGAGCGATTCTAGC | LSPPQDTSTGLEEVME |
| | | | CAGCCAATGCTCCTGAGAGTCGTGGGCTCTCAGACCAGCGATAGCAT | QLNNSFPSSRGRNTPG |
| | | | GGGCGAAGAGGATCTGCTCAGCCCTCCTCAGGATACAAGCACCGGCC | KPMREDTM (SEQ ID |
| | | | TGGAAGAAGTTATGGAACAGCTCAACAACAGCTTCCCCAGCAGCAGA | NO: 22) |
| | | | GGCAGAAACACCCCTGGCAAGCCCATGCGCGAAGATACCATG (SEQ ID | |
| | | | NO: 21) | |
| Micro- | ΔR2- | | ATGCTTTGGTGGGAAGAAGTAGAGGACTGTTATGAAAGAGAAGATGT | MLWWEEVEDCYERED |
| dystrophin | R15/ΔR18- | | TCAAAAGAAAACATTCACAAAATGGGTAAATGCACAATTTTCTAAGT | VQKKTFTKWVNAQFS |
| | R22/ΔCT | | TTGGGAAGCAGCATATTGAGAACCTCTTCAGTGACCTACAGGATGGG | KFGKQHIENLFSDLQD |
| | (CO) | | AGGCGCCTCCTAGACCTCCTCGAAGGCCTGACAGGGCAAAAACTGCC | GRRLLDLLEGLTGQKL |
| | | | AAAAGAAAAAGGATCCACAAGAGTTCATGCCCTGAACAATGTCAACA | PKEKGSTRVHALNNVN |
| | | | AGGCACTGCGGGTTTTGCAGAACAATAATGTTGATTTAGTGAATATTG | KALRVLQNNNVDLVNI |
| | | | GAAGTACTGACATCGTAGATGGAAATCATAAACTGACTCTTGGTTTG | GSTDIVDGNHKLTLGLI |
| | | | ATTTGGAATATAATCCTCCACTGGCAGGTCAAAAATGTAATGAAAAA | WNIILHWQVKNVMKN |
| | | | TATCATGGCTGGATTGCAACAAACCAACAGTGAAAAGATTCTCCTGA | IMAGLQQTNSEKILLS |
| | | | GCTGGGTCCGACAATCAACTCGTAATTATCCACAGGTTAATGTAATCA | WVRQSTRNYPQVNVIN |
| | | | ACTTCACCACCAGCTGGTCTGATGGCCTGGCTTTGAATGCTCTCATCC | FTTSWSDGLALNALIH |
| | | | ATAGTCATAGGCCAGACCTATTTGACTGGAATAGTGTGGTTTGCCAGC | SHRPDLFDWNSVVCQ |
| | | | AGTCAGCCACACAACGACTGGAACATGCATTCAACATCGCCAGATAT | QSATQRLEHAFNIARY |
| | | | CAATTAGGCATAGAGAAACTACTCGATCCTGAAGATGTTGATACCAC | QLGIEKLLDPEDVDTT |
| | | | CTATCCAGATAAGAAGTCCATCTTAATGTACATCACATCACTCTTCCA | YPDKKSILMYITSLFQV |
| | | | AGTTTTGCCTCAACAAGTGAGCATTGAAGCCATCCAGGAAGTGGAAA | LPQQVSIEAIQEVEMLP |
| | | | TGTTGCCAAGGCCACCTAAAGTGACTAAAGAAGAACATTTTCAGTTA | RPPKVTKEEHFQLHHQ |
| | | | CATCATCAAATGCACTATTCTCAACAGATCACGGTCAGTCTAGCACAG | MHYSQQITVSLAQGYE |
| | | | GGATATGAGAGAACTTCTTCCCCTAAGCCTCGATTCAAGAGCTATGCC | RTSSPKPRFKSYAYTQ |
| | | | TACACACAGGCTGCTTATGTCACCACCTCTGACCCTACACGGAGCCCA | AAYVTTSDPTRSPFPSQ |
| | | | TTTCCTTCACAGCATTTGGAAGCTCCTGAAGACAAGTCATTTGGCAGT | HLEAPEDKSFGSSLME |
| | | | TCATTGATGGAGAGTGAAGTAAACCTGGACCGTTATCAAACAGCTTT | SEVNLDRYQTALEEVL |
| | | | AGAAGAAGTATTATCGTGGCTTCTTTCTGCTGAGGACACATTGCAAGC | SWLLSAEDTLQAQGEI |
| | | | ACAAGGAGAGATTTCTAATGATGTGGAAGTGGTGAAAGACCAGTTTC | SNDVEVVKDQFHTHE |
| | | | ATACTCATGAGGGGTACATGATGGATTTGACAGCCCATCAGGGCCGG | GYMMDLTAHQGRVG |
| | | | GTTGGTAATATTCTACAATTGGGAAGTAAGCTGATTGGAACAGGAAA | NILQLGSKLIGTGKLSE |
| | | | ATTATCAGAAGATGAAGAAACTGAAGTACAAGAGCAGATGAATCTCC | DEETEVQEQMNLLNSR |
| | | | TAAATTCAAGATGGGAATGCCTCAGGGTAGCTAGCATGGAAAAACAA | WECLRVASMEKQSNL |
| | | | AGCAATTTACATAGAATTCACACTGTCCGTGAAGAAACGATGATGGT | HRIHTVREETMMVMT |
| | | | GATGACTGAAGACATGCCTTTGGAAATTTCTTATGTGCCTTCTACTTA | EDMPLEISYVPSTYLTE |
| | | | TTTGACTGAAATCACTCATGTCTCACAAGCCCTATTAGAAGTGGAACA | ITHVSQALLEVEQLLN |
| | | | ACTTCTCAATGCTCCTGACCTCTGTGCTAAGGACTTTGAAGATCTCTTT | APDLCAKDFEDLFKQE |
| | | | AAGCAAGAGGAGTCTCTGAAGAATATAAAAGATAGTCTACAACAAA | ESLKNIKDSLQQSSGRI |
| | | | GCTCAGGTCGGATTGACATTATTCATAGCAAGAAGACAGCAGCATTG | DIIHSKKTAALQSATPV |
| | | | CAAAGTGCAACGCCTGTGGAAAGGGTGAAGCTACAGGAAGCTCTCTC | ERVKLQEALSQLDFQW |
| | | | CCAGCTTGATTTCCAATGGGAAAAAGTTAACAAAATGTACAAGGACC | EKVNKMYKDRQGRFD |
| | | | GACAAGGGCGATTTGACAGATCTGTTGAGAAATGGCGGCGTTTTCAT | RSVEKWRRFHYDIKIF |
| | | | TATGATATAAAGATATTTAATCAGTGGCTAACAGAAGCTGAACAGTT | NQWLTEAEQFLRKTQI |
| | | | TCTCAGAAAGACACAAATTCCTGAGAATTGGGAACATGCTAAATACA | PENWEHAKYKWYLKE |
| | | | AATGGTATCTTAAGGAACTCCAGGATGGCATTGGGCAGCGGCAAACT | LQDGIGQRQTVVRTLN |
| | | | GTTGTCAGAACATTGAATGCAACTGGGGAAGAAATAATTCAGCAATC | ATGEEIIQQSSKTDASIL |
| | | | CTCAAAAACAGATGCCAGTATTCTACAGGAAAAATTGGGAAGCCTGA | QEKLGSLNLRWQEVC |
| | | | ATCTGCGGTGGCAGGAGGTCTGCAAACAGCTGTCAGACAGAAAAAA | KQLSDRKKRLEESSDQ |
| | | | GAGGCTAGAAGAAAGTTCTGACCAGTGGAAGCGTCTGCACCTTTCTC | WKRLHLSLQELLVWL |
| | | | TGCAGGAACTTCTGGTGTGGCTACAGCTGAAAGATGATGAATTAAGC | QLKDDELSRQAPIGGD |
| | | | CGGCAGGCACCTATTGGAGGCGACTTTCCAGCAGTTCAGAAGCAGAA | FPAVQKQNDVHRAFK |
| | | | CGATGTACATAGGGCCTTCAAGAGGGAATTGAAAACTAAAGAACCTG | RELKTKEPVIMSTLETV |
| | | | TAATCATGAGTACTCTTGAGACTGTACGAATATTTCTGACAGAGCAGC | RIFLTEQPLEGLEKLYQ |
| | | | CTTTGGAAGGACTAGAGAAACTCTACCAGGAGCCCAGAGAGCTGCCT | EPRELPPEERAQNVTRL |
| | | | CCTGAGGAGAGAGCCCAGAATGTCACTCGGCTTCTACGAAAGCAGGC | LRKQAEEVNTEWEKL |
| | | | TGAGGAGGTCAATACTGAGTGGGAAAAATTGAACCTGCACTCCGCTG | NLHSADWQRKIDETLE |
| | | | ACTGGCAGAGAAAAATAGATGAGACCCTTGAAAGACTCCAGGAACTT | RLQELQEATDELDLKL |
| | | | CAAGAGGCCACGGATGAGCTGGACCTCAAGCTGCGCCAAGCTGAGGT | RQAEVIKGSWQPVGDL |
| | | | GATCAAGGGATCCTGGCAGCCCGTGGGCGATCTCCTCATTGACTCTCT | LIDSLQDHLEKVKALR |
| | | | CCAAGATCACCTCGAGAAAGTCAAGGCACTTCGAGGAGAAATTGCGC | GEIAPLKENVSHVNDL |
| | | | CTCTGAAAGAGAACGTGAGCCACGTCAATGACCTTGCTCGCCAGCTT | ARQLTTLGIQLSPYNLS |
| | | | ACCACTTTGGGCATTCAGCTCTCACCGTATAACCTCAGCACTCTGGAA | TLEDLNTRWKLLQVA |
| | | | GACCTGAACACCAGATGGAAGCTTCTGCAGGTGGCCGTCGAGGACCG | VEDRVRQLHEAHRDF |
| | | | AGTCAGGCAGCTGCATGAAGCCCACAGGGACTTTGGTCCAGCATCTC | GPASQHFLSTSVQGPW |
| | | | AGCACTTTCTTTCCACGTCTGTCCAGGGTCCCTGGGAGAGAGCCATCT | ERAISPNKVPYYINHET |
| | | | CGCCAAACAAAGTGCCCTACTATATCAACCACGAGACTCAAACAACT | QTTCWDHPKMTELYQ |
| | | | TGCTGGGACCATCCCAAAATGACAGAGCTCTACCAGTCTTTAGCTGAC | SLADLNNVRFSAYRTA |
| | | | CTGAATAATGTCAGATTCTCAGCTTATAGGACTGCCATGAAACTCCGA | MKLRRLQKALCLDLLS |
| | | | AGACTGCAGAAGGCCCTTTGCTTGGATCTCTTGAGCCTGTCAGCTGCA | LSAACDALDQHNLKQ |
| | | | TGTGATGCCTTGGACCAGCACAACCTCAAGCAAAATGACCAGCCCAT | NDQPMDILQIINCLTTI |
| | | | GGATATCCTGCAGATTATTAATTGTTTGACCACTATTTATGACCGCCT | YDRLEQEHNNLVNVPL |
| | | | GGAGCAAGAGCACAACAATTTGGTCAACGTCCCTCTCTGCGTGGATA | CVDMCLNWLLNVYDT |
| | | | TGTGTCTGAACTGGCTGCTGAATGTTTATGATACGGGACGAACAGGG | GRTGRIRVLSFKTGIISL |
| | | | AGGATCCGTGTCCTGTCTTTTAAAACTGGCATCATTTCCCTGTGTAAA | CKAHLEDKYRYLFKQ |
| | | | GCACATTTGGAAGACAAGTACAGATACCTTTTCAAGCAAGTGGCAAG | VASSTGFCDQRRLGLL |
| | | | TTCAACAGGATTTTGTGACCAGCGCAGGCTGGGCCTCCTTCTGCATGA | LHDSIQIPRQLGEVASF |
| | | | TTCTATCCAAATTCCAAGACAGTTGGGTGAAGTTGCATCCTTTGGGGG | GGSNIEPSVRSCFQFAN |
| | | | CAGTAACATTGAGCCAAGTGTCCGGAGCTGCTTCCAATTTGCTAATAA | NKPEIEAALFLDWMRL |
| | | | TAAGCCAGAGATCGAAGCGGCCCTCTTCCTAGACTGGATGAGACTGG | EPQSMVWLPVLHRVA |
| | | | AACCCCAGTCCATGGTGTGGCTGCCCGTCCTGCACAGAGTGGCTGCTG | AAETAKHQAKCNICKE |
| | | | CAGAAACTGCCAAGCATCAGGCCAAATGTAACATCTGCAAAGAGTGT | CPIIGFRYRSLKHFNYDI |
| | | | CCAATCATTGGATTCAGGTACAGGAGTCTAAAGCACTTTAATTATGAC | CQSCFFSGRVAKGHK |
| | | | ATCTGCCAAAGCTGCTTTTTTTCTGGTCGAGTTGCAAAAGGCCATAAA | MHYPMVEYCTPTTSGE |
| | | | ATGCACTATCCCATGGTGGAATATTGCACTCCGACTACATCAGGAGA | DVRDFAKVLKNKFRT |
| | | | AGATGTTCGAGACTTTGCCAAGGTACTAAAAAACAAATTTCGAACCA | KRYFAKHPRMGYLPV |
| | | | AAAGGTATTTTGCGAAGCATCCCCGAATGGGCTACCTGCCAGTGCAG | QTVLEGDNMET (SEQ |
| | | | ACTGTCTTAGAGGGGGACAACATGGAAACT (SEQ ID NO: 23) | ID NO: 24) |
| Micro- | ΔH2- | | ATGCTTTGGTGGGAAGAAGTAGAGGACTGTTATGAAAGAGAAGATGT | MLWWEEVEDCYERED |
| dystrophin | R15 + H3/ | | TCAAAAGAAAACATTCACAAAATGGGTAAATGCACAATTTTCTAAGT | VQKKTFTKWVNAQFS |
| | ΔR18- | | TTGGGAAGCAGCATATTGAGAACCTCTTCAGTGACCTACAGGATGGG | KFGKQHIENLFSDLQD |
| | R23/ΔCT | | AGGCGCCTCCTAGACCTCCTCGAAGGCCTGACAGGGCAAAAACTGCC | GRRLLDLLEGLTGQKL |
| | (CO) | | AAAAGAAAAAGGATCCACAAGAGTTCATGCCCTGAACAATGTCAACA | PKEKGSTRVHALNNVN |
| | | | AGGCACTGCGGGTTTTGCAGAACAATAATGTTGATTTAGTGAATATTG | KALRVLQNNNVDLVNI |
| | | | GAAGTACTGACATCGTAGATGGAAATCATAAACTGACTCTTGGTTTG | GSTDIVDGNHKLTLGLI |
| | | | ATTTGGAATATAATCCTCCACTGGCAGGTCAAAAATGTAATGAAAAA | WNIILHWQVKNVMKN |
| | | | TATCATGGCTGGATTGCAACAAACCAACAGTGAAAAGATTCTCCTGA | IMAGLQQTNSEKILLS |
| | | | GCTGGGTCCGACAATCAACTCGTAATTATCCACAGGTTAATGTAATCA | WVRQSTRNYPQVNVIN |
| | | | ACTTCACCACCAGCTGGTCTGATGGCCTGGCTTTGAATGCTCTCATCC | FTTSWSDGLALNALIH |
| | | | ATAGTCATAGGCCAGACCTATTTGACTGGAATAGTGTGGTTTGCCAGC | SHRPDLFDWNSVVCQ |
| | | | AGTCAGCCACACAACGACTGGAACATGCATTCAACATCGCCAGATAT | QSATQRLEHAFNIARY |
| | | | CAATTAGGCATAGAGAAACTACTCGATCCTGAAGATGTTGATACCAC | QLGIEKLLDPEDVDTT |
| | | | CTATCCAGATAAGAAGTCCATCTTAATGTACATCACATCACTCTTCCA | YPDKKSILMYITSLFQV |
| | | | AGTTTTGCCTCAACAAGTGAGCATTGAAGCCATCCAGGAAGTGGAAA | LPQQVSIEAIQEVEMLP |
| | | | TGTTGCCAAGGCCACCTAAAGTGACTAAAGAAGAACATTTTCAGTTA | RPPKVTKEEHFQLHHQ |
| | | | CATCATCAAATGCACTATTCTCAACAGATCACGGTCAGTCTAGCACAG | MHYSQQITVSLAQGYE |
| | | | GGATATGAGAGAACTTCTTCCCCTAAGCCTCGATTCAAGAGCTATGCC | RTSSPKPRFKSYAYTQ |
| | | | TACACACAGGCTGCTTATGTCACCACCTCTGACCCTACACGGAGCCCA | AAYVTTSDPTRSPFPSQ |
| | | | TTTCCTTCACAGCATTTGGAAGCTCCTGAAGACAAGTCATTTGGCAGT | HLEAPEDKSFGSSLME |
| | | | TCATTGATGGAGAGTGAAGTAAACCTGGACCGTTATCAAACAGCTTT | SEVNLDRYQTALEEVL |
| | | | AGAAGAAGTATTATCGTGGCTTCTTTCTGCTGAGGACACATTGCAAGC | SWLLSAEDTLQAQGEI |
| | | | ACAAGGAGAGATTTCTAATGATGTGGAAGTGGTGAAAGACCAGTTTC | SNDVEVVKDQFHTHE |
| | | | ATACTCATGAGGGGTACATGATGGATTTGACAGCCCATCAGGGCCGG | GYMMDLTAHQGRVG |
| | | | GTTGGTAATATTCTACAATTGGGAAGTAAGCTGATTGGAACAGGAAA | NILQLGSKLIGTGKLSE |
| | | | ATTATCAGAAGATGAAGAAACTGAAGTACAAGAGCAGATGAATCTCC | DEETEVQEQMNLLNSR |
| | | | TAAATTCAAGATGGGAATGCCTCAGGGTAGCTAGCATGGAAAAACAA | WECLRVASMEKQSNL |
| | | | AGCAATTTACATAGAGTTTTAATGGATCTCCAGAATCAGAAACTGAA | HRVLMDLQNQKLKEL |
| | | | AGAGTTGAATGACTGGCTAACAAAAACAGAAGAAAGAACAAGGAAA | NDWLTKTEERTRKME |
| | | | ATGGAGGAAGAGCCTCTTGGACCTGATCTTGAAGACCTAAAACGCCA | EEPLGPDLEDLKRQVQ |
| | | | AGTACAACAACATAAGGTGCTTCAAGAAGATCTAGAACAAGAACAA | QHKVLQEDLEQEQVR |
| | | | GTCAGGGTCAATTCTCTCACTCACATGGTGGTGGTAGTTGATGAATCT | VNSLTHMVVVVDESS |
| | | | AGTGGAGATCACGCAACTGCTGCTTTGGAAGAACAACTTAAGGTATT | GDHATAALEEQLKVL |
| | | | GGGAGATCGATGGGCAAACATCTGTAGATGGACAGAAGACCGCTGG | GDRWANICRWTEDRW |
| | | | GTTCTTTTACAAGACATCCTTCTCAAATGGCAACGTCTTACTGAAGAA | VLLQDILLKWQRLTEE |
| | | | CAGTGCCTTTTTAGTGCATGGCTTTCAGAAAAAGAAGATGCAGTGAA | QCLFSAWLSEKEDAVN |
| | | | CAAGATTCACACAACTGGCTTTAAAGATCAAAATGAAATGTTATCAA | KIHTTGFKDQNEMLSS |
| | | | GTCTTCAAAAACTGGCCGTTTTAAAAGCGGATCTAGAAAAGAAAAAG | LQKLAVLKADLEKKK |
| | | | CAATCCATGGGCAAACTGTATTCACTCAAACAAGATCTTCTTTCAACA | QSMGKLYSLKQDLLST |
| | | | CTGAAGAATAAGTCAGTGACCCAGAAGACGGAAGCATGGCTGGATA | LKNKSVTQKTEAWLD |
| | | | ACTTTGCCCGGTGTTGGGATAATTTAGTCCAAAAACTTGAAAAGAGT | NFARCWDNLVQKLEK |
| | | | ACAGCACAGATTTCACAGCAGCCTGACCTAGCTCCTGGACTGACCAC | STAQISQQPDLAPGLTT |
| | | | TATTGGAGCCTCTCCTACTCAGACTGTTACTCTGGTGACACAACCTGT | IGASPTQTVTLVTQPVV |
| | | | GGTTACTAAGGAAACTGCCATCTCCAAACTAGAAATGCCATCTTCCTT | TKETAISKLEMPSSLML |
| | | | GATGTTGGAGGTACCTATTCACACTGTCCGTGAAGAAACGATGATGG | EVPIHTVREETMMVMT |
| | | | TGATGACTGAAGACATGCCTTTGGAAATTTCTTATGTGCCTTCTACTT | EDMPLEISYVPSTYLTE |
| | | | ATTTGACTGAAATCACTCATGTCTCACAAGCCCTATTAGAAGTGGAAC | ITHVSQALLEVEQLLN |
| | | | AACTTCTCAATGCTCCTGACCTCTGTGCTAAGGACTTTGAAGATCTCT | APDLCAKDFEDLFKQE |
| | | | TTAAGCAAGAGGAGTCTCTGAAGAATATAAAAGATAGTCTACAACAA | ESLKNIKDSLQQSSGRI |
| | | | AGCTCAGGTCGGATTGACATTATTCATAGCAAGAAGACAGCAGCATT | DIIHSKKTAALQSATPV |
| | | | GCAAAGTGCAACGCCTGTGGAAAGGGTGAAGCTACAGGAAGCTCTCT | ERVKLQEALSQLDFQW |
| | | | CCCAGCTTGATTTCCAATGGGAAAAAGTTAACAAAATGTACAAGGAC | EKVNKMYKDRQGRFD |
| | | | CGACAAGGGCGATTTGACAGATCTGTTGAGAAATGGCGGCGTTTTCA | RSVEKWRRFHYDIKIF |
| | | | TTATGATATAAAGATATTTAATCAGTGGCTAACAGAAGCTGAACAGT | NQWLTEAEQFLRKTQI |
| | | | TTCTCAGAAAGACACAAATTCCTGAGAATTGGGAACATGCTAAATAC | PENWEHAKYKWYLKE |
| | | | AAATGGTATCTTAAGGAACTCCAGGATGGCATTGGGCAGCGGCAAAC | LQDGIGQRQTVVRTLN |
| | | | TGTTGTCAGAACATTGAATGCAACTGGGGAAGAAATAATTCAGCAAT | ATGEEIIQQSSKTDASIL |
| | | | CCTCAAAAACAGATGCCAGTATTCTACAGGAAAAATTGGGAAGCCTG | QEKLGSLNLRWQEVC |
| | | | AATCTGCGGTGGCAGGAGGTCTGCAAACAGCTGTCAGACAGAAAAA | KQLSDRKKRLEETLER |
| | | | AGAGGCTAGAAGAAACCCTTGAAAGACTCCAGGAACTTCAAGAGGC | LQELQEATDELDLKLR |
| | | | CACGGATGAGCTGGACCTCAAGCTGCGCCAAGCTGAGGTGATCAAGG | QAEVIKGSWQPVGDLL |
| | | | GATCCTGGCAGCCCGTGGGCGATCTCCTCATTGACTCTCTCCAAGATC | IDSLQDHLEKVKALRG |
| | | | ACCTCGAGAAAGTCAAGGCACTTCGAGGAGAAATTGCGCCTCTGAAA | EIAPLKENVSHVNDLA |
| | | | GAGAACGTGAGCCACGTCAATGACCTTGCTCGCCAGCTTACCACTTTG | RQLTTLGIQLSPYNLST |
| | | | GGCATTCAGCTCTCACCGTATAACCTCAGCACTCTGGAAGACCTGAAC | LEDLNTRWKLLQVAV |
| | | | ACCAGATGGAAGCTTCTGCAGGTGGCCGTCGAGGACCGAGTCAGGCA | EDRVRQLHEAHRDFGP |
| | | | GCTGCATGAAGCCCACAGGGACTTTGGTCCAGCATCTCAGCACTTTCT | ASQHELSTSVQGPWER |
| | | | TTCCACGTCTGTCCAGGGTCCCTGGGAGAGAGCCATCTCGCCAAACA | AISPNKVPYYINHETQT |
| | | | AAGTGCCCTACTATATCAACCACGAGACTCAAACAACTTGCTGGGAC | TCWDHPKMTELYQSL |
| | | | CATCCCAAAATGACAGAGCTCTACCAGTCTTTAGCTGACCTGAATAAT | ADLNNVRFSAYRTAM |
| | | | GTCAGATTCTCAGCTTATAGGACTGCCATGAAACTCCGAAGACTGCA | KLRRLQKALCLDLLSL |
| | | | GAAGGCCCTTTGCTTGGATCTCTTGAGCCTGTCAGCTGCATGTGATGC | SAACDALDQHNLKQN |
| | | | CTTGGACCAGCACAACCTCAAGCAAAATGACCAGCCCATGGATATCC | DQPMDILQIINCLTTIY |
| | | | TGCAGATTATTAATTGTTTGACCACTATTTATGACCGCCTGGAGCAAG | DRLEQEHNNLVNVPLC |
| | | | AGCACAACAATTTGGTCAACGTCCCTCTCTGCGTGGATATGTGTCTGA | VDMCLNWLLNVYDTG |
| | | | ACTGGCTGCTGAATGTTTATGATACGGGACGAACAGGGAGGATCCGT | RTGRIRVLSFKTGIISLC |
| | | | GTCCTGTCTTTTAAAACTGGCATCATTTCCCTGTGTAAAGCACATTTG | KAHLEDKYRYLFKQV |
| | | | GAAGACAAGTACAGATACCTTTTCAAGCAAGTGGCAAGTTCAACAGG | ASSTGFCDQRRLGLLL |
| | | | ATTTTGTGACCAGCGCAGGCTGGGCCTCCTTCTGCATGATTCTATCCA | HDSIQIPRQLGEVASFG |
| | | | AATTCCAAGACAGTTGGGTGAAGTTGCATCCTTTGGGGGCAGTAACA | GSNIEPSVRSCFQFANN |
| | | | TTGAGCCAAGTGTCCGGAGCTGCTTCCAATTTGCTAATAATAAGCCAG | KPEIEAALFLDWMRLE |
| | | | AGATCGAAGCGGCCCTCTTCCTAGACTGGATGAGACTGGAACCCCAG | PQSMVWLPVLHRVAA |
| | | | TCCATGGTGTGGCTGCCCGTCCTGCACAGAGTGGCTGCTGCAGAAACT | AETAKHQAKCNICKEC |
| | | | GCCAAGCATCAGGCCAAATGTAACATCTGCAAAGAGTGTCCAATCAT | PIIGFRYRSLKHFNYDIC |
| | | | TGGATTCAGGTACAGGAGTCTAAAGCACTTTAATTATGACATCTGCCA | QSCFFSGRVAKGHKM |
| | | | AAGCTGCTTTTTTTCTGGTCGAGTTGCAAAAGGCCATAAAATGCACTA | HYPMVEYCTPTTSGED |
| | | | TCCCATGGTGGAATATTGCACTCCGACTACATCAGGAGAAGATGTTC | VRDFAKVLKNKFRTKR |
| | | | GAGACTTTGCCAAGGTACTAAAAAACAAATTTCGAACCAAAAGGTAT | YFAKHPRMGYLPVQT |
| | | | TTTGCGAAGCATCCCCGAATGGGCTACCTGCCAGTGCAGACTGTCTTA | VLEGDNMET (SEQ ID |
| | | | GAGGGGGACAACATGGAAACT (SEQ ID NO: 25) | NO: 26) |
| Mini- | ΔH2- | | ATGCTTTGGTGGGAAGAAGTCGAGGACTGCTACGAGCGCGAGGACGT | MLWWEEVEDCYERED |
| dystrophin | R15/ΔR18- | | GCAGAAGAAAACCTTCACCAAATGGGTCAACGCCCAGTTCAGCAAGT | VQKKTFTKWVNAQFS |
| | R19/ΔCT | | TCGGCAAGCAGCACATCGAGAACCTGTTCAGCGACCTGCAGGATGGC | KFGKQHIENLFSDLQD |
| | (CO) | | AGAAGGCTGCTGGATCTGCTGGAAGGCCTGACAGGACAGAAGCTGCC | GRRLLDLLEGLTGQKL |
| | | | CAAAGAGAAGGGCAGCACAAGAGTGCACGCCCTGAACAACGTGAAC | PKEKGSTRVHALNNVN |
| | | | AAGGCCCTGAGAGTGCTGCAGAACAACAACGTGGACCTGGTCAACAT | KALRVLQNNNVDLVNI |
| | | | CGGCAGCACCGACATCGTGGACGGCAACCACAAACTGACCCTGGGCC | GSTDIVDGNHKLTLGLI |
| | | | TGATCTGGAACATCATCCTGCACTGGCAAGTGAAGAACGTGATGAAG | WNIILHWQVKNVMKN |
| | | | AACATCATGGCCGGCCTGCAGCAGACCAACAGCGAGAAGATTCTGCT | IMAGLQQTNSEKILLS |
| | | | GAGCTGGGTCCGACAGAGCACCCGGAATTACCCTCAAGTGAACGTGA | WVRQSTRNYPQVNVIN |
| | | | TCAACTTCACCACCTCTTGGAGCGACGGACTGGCCCTGAATGCCCTGA | FTTSWSDGLALNALIH |
| | | | TCCACAGCCACAGACCTGACCTGTTCGACTGGAACAGCGTCGTGTGTC | SHRPDLFDWNSVVCQ |
| | | | AGCAGAGCGCCACACAGAGGCTGGAACACGCCTTCAATATCGCCAGA | QSATQRLEHAFNIARY |
| | | | TACCAGCTGGGCATCGAGAAACTGCTGGACCCCGAGGATGTGGACAC | QLGIEKLLDPEDVDTT |
| | | | CACCTATCCTGACAAGAAATCCATCCTCATGTACATCACCAGCCTGTT | YPDKKSILMYITSLFQV |
| | | | CCAGGTGCTGCCCCAGCAGGTTTCCATCGAGGCCATTCAAGAGGTCG | LPQQVSIEAIQEVEMLP |
| | | | AGATGCTGCCCAGACCTCCTAAAGTGACCAAAGAGGAACACTTCCAG | RPPKVTKEEHFQLHHQ |
| | | | CTGCACCACCAGATGCACTACTCTCAGCAGATCACCGTGTCTCTGGCC | MHYSQQITVSLAQGYE |
| | | | CAGGGCTACGAGAGAACAAGCAGCCCCAAGCCTCGGTTCAAGAGCTA | RTSSPKPRFKSYAYTQ |
| | | | CGCCTATACACAGGCCGCCTACGTGACCACCAGCGATCCTACAAGAA | AAYVTTSDPTRSPFPSQ |
| | | | GCCCATTTCCTAGCCAGCACCTCGAGGCCCCTGAGGATAAGAGCTTTG | HLEAPEDKSFGSSLME |
| | | | GCAGCAGCCTGATGGAAAGCGAAGTGAACCTGGACCGCTACCAGAC | SEVNLDRYQTALEEVL |
| | | | AGCCCTGGAAGAGGTTCTGAGCTGGCTGCTGTCTGCCGAGGATACAC | SWLLSAEDTLQAQGEI |
| | | | TGCAGGCTCAGGGCGAGATCAGCAACGACGTGGAAGTGGTCAAGGA | SNDVEVVKDQFHTHE |
| | | | CCAGTTTCACACCCACGAGGGCTACATGATGGACCTGACAGCCCACC | GYMMDLTAHQGRVG |
| | | | AGGGCAGAGTGGGCAATATTCTGCAGCTGGGCTCCAAGCTGATCGGC | NILQLGSKLIGTGKLSE |
| | | | ACAGGCAAGCTGAGCGAGGACGAAGAGACAGAGGTGCAAGAGCAGA | DEETEVQEQMNLLNSR |
| | | | TGAACCTGCTGAACAGCAGATGGGAGTGTCTGAGAGTGGCCAGCATG | WECLRVASMEKQSNL |
| | | | GAAAAGCAGAGCAACCTGCACCGGGTGCTGATGGATCTCCAGAACCA | HRVLMDLQNQKLKEL |
| | | | GAAGCTGAAAGAGCTGAACGACTGGCTGACCAAGACCGAGGAACGG | NDWLTKTEERTRKME |
| | | | ACCCGGAAGATGGAAGAGGAACCTCTGGGACCCGACCTGGAAGATCT | EEPLGPDLEDLKRQVQ |
| | | | GAAAAGACAGGTGCAGCAGCATAAGGTGCTGCAAGAGGACCTCGAG | QHKVLQEDLEQEQVR |
| | | | CAAGAGCAAGTGCGCGTGAACAGCCTGACACACATGGTGGTGGTCGT | VNSLTHMVVVVDESS |
| | | | GGATGAGAGCAGCGGAGATCATGCCACAGCCGCTCTGGAAGAACAG | GDHATAALEEQLKVL |
| | | | CTGAAGGTGCTGGGAGACAGATGGGCCAATATCTGCCGGTGGACCGA | GDRWANICRWTEDRW |
| | | | GGATAGATGGGTGCTGCTCCAGGACATCCTGCTGAAGTGGCAGCGGC | VLLQDILLKWQRLTEE |
| | | | TGACAGAGGAACAGTGCCTGTTTAGCGCCTGGCTGTCCGAGAAAGAG | QCLFSAWLSEKEDAVN |
| | | | GACGCCGTCAACAAGATCCACACCACCGGCTTCAAGGATCAGAATGA | KIHTTGFKDQNEMLSS |
| | | | GATGCTGAGCAGCCTGCAGAAACTGGCCGTGCTGAAGGCTGACCTGG | LQKLAVLKADLEKKK |
| | | | AAAAGAAAAAGCAGTCCATGGGCAAGCTGTACTCCCTGAAGCAGGA | QSMGKLYSLKQDLLST |
| | | | CCTGCTGAGCACACTGAAGAACAAGAGCGTGACCCAGAAAACCGAG | LKNKSVTQKTEAWLD |
| | | | GCCTGGCTGGACAACTTCGCCAGATGCTGGGACAACCTGGTGCAGAA | NFARCWDNLVQKLEK |
| | | | GCTGGAAAAGTCCACCGCTCAGATCAGCCAAGAGATCAGCTACGTGC | STAQISQEISYVPSTYLT |
| | | | CCAGCACATACCTGACCGAGATCACCCATGTGTCACAGGCCCTGCTTG | EITHVSQALLEVEQLLN |
| | | | AGGTGGAACAGCTGCTGAACGCCCCTGATCTGTGCGCCAAGGACTTC | APDLCAKDFEDLFKQE |
| | | | GAGGATCTGTTCAAGCAAGAGGAAAGCCTGAAGAATATCAAGGACTC | ESLKNIKDSLQQSSGRI |
| | | | TCTGCAGCAGTCCAGCGGCCGGATCGATATCATCCACAGCAAGAAAA | DIIHSKKTAALQSATPV |
| | | | CCGCCGCACTGCAGTCCGCCACACCTGTGGAAAGAGTGAAGCTGCAA | ERVKLQEALSQLDFQW |
| | | | GAAGCTCTGAGCCAGCTGGACTTCCAGTGGGAGAAAGTGAACAAGAT | EKVNKMYKDRQGRFD |
| | | | GTACAAGGACCGGCAGGGCAGATTCGACCGCAGCGTTGAAAAGTGG | RSVEKWRRFHYDIKIF |
| | | | CGGCGGTTCCACTACGACATCAAGATCTTCAACCAGTGGCTGACCGA | NQWLTEAEQFLRKTQI |
| | | | GGCCGAACAGTTCCTGAGAAAGACACAGATCCCCGAGAACTGGGAG | PENWEHAKYKWYLKE |
| | | | CACGCCAAGTACAAGTGGTATCTGAAAGAACTGCAGGACGGCATCGG | LQDGIGQRQTVVRTLN |
| | | | CCAGAGGCAGACAGTCGTCAGAACACTGAATGCCACCGGCGAGGAA | ATGEEIIQQSSKTDASIL |
| | | | ATCATCCAGCAGAGCAGCAAGACCGACGCCAGCATCCTGCAAGAGA | QEKLGSLNLRWQEVC |
| | | | AGCTGGGAAGCCTGAACCTGCGGTGGCAAGAAGTGTGCAAGCAGCTG | KQLSDRKKRLEEQPDL |
| | | | TCCGACCGGAAGAAGAGACTCGAGGAACAGCCCGATCTGGCCCCTGG | APGLTTIGASPTQTVTL |
| | | | CCTGACAACAATTGGAGCTAGCCCCACACAGACCGTGACACTGGTTA | VTQPVVTKETAISKLE |
| | | | CACAGCCCGTGGTCACCAAAGAGACAGCCATCAGCAAACTGGAAATG | MPSSLMLEVPALADFN |
| | | | CCCTCTAGCCTGATGCTCGAAGTGCCCGCACTGGCCGACTTCAATAGA | RAWTELTDWLSLLDQ |
| | | | GCCTGGACCGAGCTGACAGATTGGCTGAGCCTCCTGGACCAAGTGAT | VIKSQRVMVGDLEDIN |
| | | | CAAGAGCCAGAGAGTGATGGTCGGAGATCTCGAGGACATCAACGAG | EMIIKQKATMQDLEQR |
| | | | ATGATCATTAAGCAGAAAGCCACCATGCAGGATCTCGAGCAGAGAAG | RPQLEELITAAQNLKN |
| | | | GCCTCAGCTCGAAGAACTGATCACCGCCGCTCAGAATCTGAAAAACA | KTSNQEARTIITDRIERI |
| | | | AGACCAGCAATCAAGAGGCCCGGACCATCATCACCGACCGGATCGAG | QNQWDEVQEHLQNRR |
| | | | AGAATCCAGAATCAGTGGGACGAAGTCCAAGAGCATCTGCAGAATCG | QQLNEMLKDSTQWLE |
| | | | GCGGCAGCAGCTCAACGAAATGCTGAAGGATAGCACCCAGTGGCTCG | AKEEAEQVLGQARAK |
| | | | AGGCCAAAGAAGAGGCAGAACAGGTCCTCGGACAGGCTAGGGCCAA | LESWKEGPYTVDAIQK |
| | | | GCTGGAATCTTGGAAAGAGGGCCCCTACACCGTCGACGCCATCCAGA | KITETKQLAKDLRQWQ |
| | | | AGAAGATCACCGAGACAAAGCAGCTGGCCAAGGACCTGAGACAGTG | TNVDVANDLALKLLR |
| | | | GCAGACCAATGTGGACGTGGCCAACGATCTGGCTCTGAAGCTGCTGA | DYSADDTRKVHMITEN |
| | | | GGGACTACAGCGCCGACGACACCAGAAAGGTGCACATGATCACAGA | INASWRSIHKRVSEREA |
| | | | GAACATCAACGCCTCTTGGCGGAGCATCCACAAGCGGGTGTCAGAGA | ALEETHRLLQQFPLDL |
| | | | GAGAGGCCGCTCTCGAGGAAACCCACAGACTGCTTCAGCAGTTCCCT | EKFLAWLTEAETTANV |
| | | | CTGGACCTCGAAAAGTTCCTGGCCTGGCTCACAGAGGCCGAGACAAC | LQDATRKERLLEDSKG |
| | | | AGCTAACGTCCTCCAGGATGCCACCAGAAAAGAGAGACTGCTCGAGG | VKELMKQWQDLQGEI |
| | | | ACTCCAAGGGCGTCAAAGAACTCATGAAGCAGTGGCAGGATCTGCAG | EAHTDVYHNLDENSQ |
| | | | GGCGAAATCGAGGCCCACACCGACGTGTACCACAACCTGGACGAGA | KILRSLEGSDDAVLLQ |
| | | | ACTCCCAGAAGATCCTGCGGAGCCTGGAAGGCAGTGATGATGCTGTG | RRLDNMNFKWSELRK |
| | | | CTCCTCCAGCGGAGACTGGACAACATGAACTTCAAGTGGAGCGAGCT | KSLNIRSHLEASSDQW |
| | | | GCGCAAGAAGTCCCTGAACATCAGAAGCCACCTGGAAGCCAGCAGC | KRLHLSLQELLVWLQL |
| | | | GACCAGTGGAAAAGACTGCATCTGTCCCTGCAAGAACTGCTCGTGTG | KDDELSRQAPIGGDFP |
| | | | GCTGCAGCTCAAGGACGACGAACTGAGCAGACAGGCCCCTATCGGCG | AVQKQNDVHRAFKRE |
| | | | GAGATTTTCCCGCCGTGCAGAAACAGAACGACGTGCACAGAGCCTTC | LKTKEPVIMSTLETVRI |
| | | | AAGCGCGAGCTGAAAACAAAAGAACCCGTCATCATGAGCACCCTGG | FLTEQPLEGLEKLYQEP |
| | | | AAACCGTGCGGATCTTTCTGACCGAGCAGCCCCTGGAAGGACTGGAA | RELPPEERAQNVTRLL |
| | | | AAGCTGTACCAAGAGCCTCGCGAGCTGCCTCCTGAAGAAAGGGCCCA | RKQAEEVNTEWEKLN |
| | | | GAATGTGACCCGGCTGCTGAGAAAGCAGGCCGAGGAAGTGAATACC | LHSADWQRKIDETLER |
| | | | GAATGGGAGAAGCTCAATCTGCACTCCGCCGACTGGCAGCGGAAGAT | LRELQEATDELDLKLR |
| | | | CGATGAGACACTGGAACGGCTGCGGGAATTGCAAGAGGCCACCGAT | QAEVIKGSWQPVGDLL |
| | | | GAGCTGGACCTGAAGCTGAGACAGGCCGAAGTCATCAAAGGATCCTG | IDSLQDHLEKVKALRG |
| | | | GCAGCCTGTGGGCGACCTGCTCATCGATAGTCTGCAGGACCACCTCG | EIAPLKENVSHVNDLA |
| | | | AGAAAGTCAAAGCCCTGCGGGGAGAGATCGCCCCACTGAAAGAAAA | RQLTTLGIQLSPYNLST |
| | | | CGTGTCCCACGTGAACGACCTGGCCAGACAGCTGACAACACTGGGCA | LEDLNTRWKLLQVAV |
| | | | TCCAGCTGAGCCCCTACAACCTGTCCACACTTGAGGACCTGAACACCC | EDRVRQLHEAHRDFGP |
| | | | GGTGGAAACTGCTCCAAGTGGCCGTGGAAGATAGAGTGCGGCAGCTG | ASQHELSTSVQGPWER |
| | | | CACGAAGCCCACAGAGATTTTGGACCAGCCAGCCAGCACTTCCTGAG | AISPNKVPYYINHETQT |
| | | | CACCTCAGTTCAAGGCCCCTGGGAGAGAGCTATCAGCCCTAACAAGG | TCWDHPKMTELYQSL |
| | | | TGCCCTACTACATCAACCACGAGACACAGACCACCTGTTGGGATCAC | ADLNNVRFSAYRTAM |
| | | | CCCAAGATGACCGAACTGTATCAGTCCCTGGCCGATCTGAACAATGT | KLRRLQKALCLDLLSL |
| | | | GCGGTTCAGCGCCTACCGGACCGCTATGAAGCTGCGAAGGCTGCAGA | SAACDALDQHNLKQN |
| | | | AAGCTCTGTGCCTGGACCTCCTGTCTCTGAGCGCTGCTTGTGATGCCC | DQPMDILQIINCLTTIY |
| | | | TGGACCAGCATAATCTGAAGCAGAACGACCAGCCTATGGACATCCTC | DRLEQEHNNLVNVPLC |
| | | | CAGATCATCAATTGCCTGACCACCATCTACGACCGCCTGGAACAAGA | VDMCLNWLLNVYDTG |
| | | | ACACAACAACCTCGTGAACGTGCCCCTGTGCGTGGACATGTGCCTGA | RTGRIRVLSFKTGIISLC |
| | | | ATTGGCTGCTGAATGTGTACGACACCGGCCGCACAGGCAGAATCAGA | KAHLEDKYRYLFKQV |
| | | | GTGCTGTCCTTCAAGACCGGCATCATCTCCCTGTGCAAGGCCCATCTT | ASSTGFCDQRRLGLLL |
| | | | GAGGACAAGTACCGCTACCTGTTCAAACAGGTGGCCTCCAGCACCGG | HDSIQIPRQLGEVASFG |
| | | | CTTTTGCGACCAAAGAAGGCTGGGACTGCTCCTGCACGACAGCATTC | GSNIEPSVRSCFQFANN |
| | | | AGATCCCTAGACAGCTGGGCGAAGTGGCCTCCTTTGGCGGCTCTAAT | KPEIEAALFLDWMRLE |
| | | | ATCGAGCCTAGCGTGCGGAGCTGCTTCCAGTTCGCCAACAACAAGCC | PQSMVWLPVLHRVAA |
| | | | CGAGATCGAGGCTGCCCTGTTCCTGGACTGGATGAGACTGGAACCCC | AETAKHQAKCNICKEC |
| | | | AGAGCATGGTCTGGCTGCCAGTGCTGCATAGAGTGGCCGCTGCCGAA | PIIGFRYRSLKHFNYDIC |
| | | | ACAGCCAAGCACCAGGCCAAGTGCAACATCTGCAAAGAGTGCCCCAT | QSCFFSGRVAKGHKM |
| | | | CATCGGCTTCCGGTACAGATCTCTGAAGCACTTCAACTACGATATCTG | HYPMVEYC (SEQ ID |
| | | | CCAGAGCTGTTTCTTCAGCGGCAGAGTCGCCAAGGGCCACAAAATGC | NO: 28) |
| | | | ACTACCCCATGGTGGAATACTGC (SEQ ID NO: 27) | |
| Mini- | ΔH2- | | ATGCTTTGGTGGGAAGAAGTCGAGGACTGCTACGAGCGCGAGGACGT | MLWWEEVEDCYERED |
| dystrophin | R15 + H3/ | | GCAGAAGAAAACCTTCACCAAATGGGTCAACGCCCAGTTCAGCAAGT | VQKKTFTKWVNAQFS |
| | ΔR18- | | TCGGCAAGCAGCACATCGAGAACCTGTTCAGCGACCTGCAGGATGGC | KFGKQHIENLFSDLQD |
| | R19/ΔCT | | AGAAGGCTGCTGGATCTGCTGGAAGGCCTGACAGGACAGAAGCTGCC | GRRLLDLLEGLTGQKL |
| | | | CAAAGAGAAGGGCAGCACAAGAGTGCACGCCCTGAACAACGTGAAC | PKEKGSTRVHALNNVN |
| | | | AAGGCCCTGAGAGTGCTGCAGAACAACAACGTGGACCTGGTCAACAT | KALRVLQNNNVDLVNI |
| | | | CGGCAGCACCGACATCGTGGACGGCAACCACAAACTGACCCTGGGCC | GSTDIVDGNHKLTLGLI |
| | | | TGATCTGGAACATCATCCTGCACTGGCAAGTGAAGAACGTGATGAAG | WNIILHWQVKNVMKN |
| | | | AACATCATGGCCGGCCTGCAGCAGACCAACAGCGAGAAGATTCTGCT | IMAGLQQTNSEKILLS |
| | | | GAGCTGGGTCCGACAGAGCACCCGGAATTACCCTCAAGTGAACGTGA | WVRQSTRNYPQVNVIN |
| | | | TCAACTTCACCACCTCTTGGAGCGACGGACTGGCCCTGAATGCCCTGA | FTTSWSDGLALNALIH |
| | | | TCCACAGCCACAGACCTGACCTGTTCGACTGGAACAGCGTCGTGTGTC | SHRPDLFDWNSVVCQ |
| | | | AGCAGAGCGCCACACAGAGGCTGGAACACGCCTTCAATATCGCCAGA | QSATQRLEHAFNIARY |
| | | | TACCAGCTGGGCATCGAGAAACTGCTGGACCCCGAGGATGTGGACAC | QLGIEKLLDPEDVDTT |
| | | | CACCTATCCTGACAAGAAATCCATCCTCATGTACATCACCAGCCTGTT | YPDKKSILMYITSLFQV |
| | | | CCAGGTGCTGCCCCAGCAGGTTTCCATCGAGGCCATTCAAGAGGTCG | LPQQVSIEAIQEVEMLP |
| | | | AGATGCTGCCCAGACCTCCTAAAGTGACCAAAGAGGAACACTTCCAG | RPPKVTKEEHFQLHHQ |
| | | | CTGCACCACCAGATGCACTACTCTCAGCAGATCACCGTGTCTCTGGCC | MHYSQQITVSLAQGYE |
| | | | CAGGGCTACGAGAGAACAAGCAGCCCCAAGCCTCGGTTCAAGAGCTA | RTSSPKPRFKSYAYTQ |
| | | | CGCCTATACACAGGCCGCCTACGTGACCACCAGCGATCCTACAAGAA | AAYVTTSDPTRSPFPSQ |
| | | | GCCCATTTCCTAGCCAGCACCTCGAGGCCCCTGAGGATAAGAGCTTTG | HLEAPEDKSFGSSLME |
| | | | GCAGCAGCCTGATGGAAAGCGAAGTGAACCTGGACCGCTACCAGAC | SEVNLDRYQTALEEVL |
| | | | AGCCCTGGAAGAGGTTCTGAGCTGGCTGCTGTCTGCCGAGGATACAC | SWLLSAEDTLQAQGEI |
| | | | TGCAGGCTCAGGGCGAGATCAGCAACGACGTGGAAGTGGTCAAGGA | SNDVEVVKDQFHTHE |
| | | | CCAGTTTCACACCCACGAGGGCTACATGATGGACCTGACAGCCCACC | GYMMDLTAHQGRVG |
| | | | AGGGCAGAGTGGGCAATATTCTGCAGCTGGGCTCCAAGCTGATCGGC | NILQLGSKLIGTGKLSE |
| | | | ACAGGCAAGCTGAGCGAGGACGAAGAGACAGAGGTGCAAGAGCAGA | DEETEVQEQMNLLNSR |
| | | | TGAACCTGCTGAACAGCAGATGGGAGTGTCTGAGAGTGGCCAGCATG | WECLRVASMEKQSNL |
| | | | GAAAAGCAGAGCAACCTGCACCGGGTGCTGATGGATCTCCAGAACCA | HRVLMDLQNQKLKEL |
| | | | GAAGCTGAAAGAGCTGAACGACTGGCTGACCAAGACCGAGGAACGG | NDWLTKTEERTRKME |
| | | | ACCCGGAAGATGGAAGAGGAACCTCTGGGACCCGACCTGGAAGATCT | EEPLGPDLEDLKRQVQ |
| | | | GAAAAGACAGGTGCAGCAGCATAAGGTGCTGCAAGAGGACCTCGAG | QHKVLQEDLEQEQVR |
| | | | CAAGAGCAAGTGCGCGTGAACAGCCTGACACACATGGTGGTGGTCGT | VNSLTHMVVVVDESS |
| | | | GGATGAGAGCAGCGGAGATCATGCCACAGCCGCTCTGGAAGAACAG | GDHATAALEEQLKVL |
| | | | CTGAAGGTGCTGGGAGACAGATGGGCCAATATCTGCCGGTGGACCGA | GDRWANICRWTEDRW |
| | | | GGATAGATGGGTGCTGCTCCAGGACATCCTGCTGAAGTGGCAGCGGC | VLLQDILLKWQRLTEE |
| | | | TGACAGAGGAACAGTGCCTGTTTAGCGCCTGGCTGTCCGAGAAAGAG | QCLFSAWLSEKEDAVN |
| | | | GACGCCGTCAACAAGATCCACACCACCGGCTTCAAGGATCAGAATGA | KIHTTGFKDQNEMLSS |
| | | | GATGCTGAGCAGCCTGCAGAAACTGGCCGTGCTGAAGGCTGACCTGG | LQKLAVLKADLEKKK |
| | | | AAAAGAAAAAGCAGTCCATGGGCAAGCTGTACTCCCTGAAGCAGGA | QSMGKLYSLKQDLLST |
| | | | CCTGCTGAGCACACTGAAGAACAAGAGCGTGACCCAGAAAACCGAG | LKNKSVTQKTEAWLD |
| | | | GCCTGGCTGGACAACTTCGCCAGATGCTGGGACAACCTGGTGCAGAA | NFARCWDNLVQKLEK |
| | | | GCTGGAAAAGTCCACCGCTCAGATCAGTCAGCAGCCCGATCTGGCAC | STAQISQQPDLAPGLTT |
| | | | CTGGCCTGACAACAATTGGCGCTAGCCCCACACAGACCGTGACACTG | IGASPTQTVTLVTQPVV |
| | | | GTTACACAGCCCGTGGTCACCAAAGAGACAGCCATCAGCAAACTGGA | TKETAISKLEMPSSLML |
| | | | AATGCCCTCTAGCCTGATGCTCGAAGTGCCCGAGATCTCCTACGTGCC | EVPEISYVPSTYLTEITH |
| | | | CAGCACATACCTGACCGAGATCACCCATGTGTCACAGGCCCTGCTTG | VSQALLEVEQLLNAPD |
| | | | AGGTGGAACAGCTGCTGAACGCCCCTGATCTGTGCGCCAAGGACTTC | LCAKDFEDLFKQEESL |
| | | | GAGGATCTGTTCAAGCAAGAGGAAAGCCTGAAGAATATCAAGGACTC | KNIKDSLQQSSGRIDIIH |
| | | | TCTGCAGCAGTCCAGCGGCCGGATCGATATCATCCACAGCAAGAAAA | SKKTAALQSATPVERV |
| | | | CCGCCGCACTGCAGTCCGCCACACCTGTGGAAAGAGTGAAGCTGCAA | KLQEALSQLDFQWEK |
| | | | GAAGCTCTGAGCCAGCTGGACTTCCAGTGGGAGAAAGTGAACAAGAT | VNKMYKDRQGRFDRS |
| | | | GTACAAGGACCGGCAGGGCAGATTCGACCGCAGCGTTGAAAAGTGG | VEKWRRFHYDIKIFNQ |
| | | | CGGCGGTTCCACTACGACATCAAGATCTTCAACCAGTGGCTGACCGA | WLTEAEQFLRKTQIPE |
| | | | GGCCGAACAGTTCCTGAGAAAGACACAGATCCCCGAGAACTGGGAG | NWEHAKYKWYLKEL |
| | | | CACGCCAAGTACAAGTGGTATCTGAAAGAACTGCAGGACGGCATCGG | QDGIGQRQTVVRTLNA |
| | | | CCAGAGGCAGACAGTCGTCAGAACACTGAATGCCACCGGCGAGGAA | TGEEIIQQSSKTDASILQ |
| | | | ATCATCCAGCAGAGCAGCAAGACCGACGCCAGCATCCTGCAAGAGA | EKLGSLNLRWQEVCK |
| | | | AGCTGGGAAGCCTGAACCTGCGGTGGCAAGAAGTGTGCAAGCAGCTG | QLSDRKKRLEEALADF |
| | | | TCCGACCGGAAGAAGAGACTGGAAGAAGCCCTGGCCGACTTCAACA | NRAWTELTDWLSLLD |
| | | | GAGCCTGGACCGAACTGACCGATTGGCTGAGCCTCCTGGACCAAGTG | QVIKSQRVMVGDLEDI |
| | | | ATCAAGAGCCAGAGAGTGATGGTCGGAGATCTTGAGGACATCAACGA | NEMIIKQKATMQDLEQ |
| | | | GATGATCATTAAGCAGAAAGCCACCATGCAGGATCTCGAGCAGAGAA | RRPQLEELITAAQNLK |
| | | | GGCCTCAGCTCGAGGAACTGATCACCGCCGCTCAGAATCTGAAAAAC | NKTSNQEARTIITDRIE |
| | | | AAGACCAGCAATCAAGAGGCCCGGACCATCATCACCGACCGGATCGA | RIQNQWDEVQEHLQN |
| | | | GAGAATCCAGAATCAGTGGGACGAAGTCCAAGAGCATCTGCAGAATC | RRQQLNEMLKDSTQW |
| | | | GGCGGCAGCAGCTCAACGAAATGCTGAAGGATAGCACCCAGTGGCTC | LEAKEEAEQVLGQARA |
| | | | GAGGCCAAAGAAGAGGCAGAACAGGTCCTCGGACAGGCTAGGGCCA | KLESWKEGPYTVDAIQ |
| | | | AGCTGGAATCTTGGAAAGAGGGCCCCTACACCGTCGACGCCATCCAG | KKITETKQLAKDLRQW |
| | | | AAGAAGATCACCGAGACAAAGCAGCTGGCCAAGGACCTGAGACAGT | QTNVDVANDLALKLL |
| | | | GGCAGACCAATGTGGACGTGGCCAATGATCTGGCTCTGAAGCTGCTG | RDYSADDTRKVHMITE |
| | | | AGGGACTACAGCGCCGACGACACCAGAAAGGTGCACATGATCACAG | NINASWRSIHKRVSERE |
| | | | AGAACATCAACGCCTCTTGGCGGAGCATCCACAAGCGGGTGTCAGAG | AALEETHRLLQQFPLD |
| | | | AGAGAGGCCGCTCTCGAGGAAACCCACAGACTGCTTCAGCAGTTCCC | LEKFLAWLTEAETTAN |
| | | | TCTGGACCTCGAAAAGTTCCTGGCCTGGCTCACAGAGGCCGAGACAA | VLQDATRKERLLEDSK |
| | | | CAGCTAACGTCCTCCAGGATGCCACCAGAAAAGAGAGACTGCTCGAG | GVKELMKQWQDLQGE |
| | | | GACAGCAAGGGCGTCAAAGAACTCATGAAGCAGTGGCAGGATCTGC | IEAHTDVYHNLDENSQ |
| | | | AGGGCGAAATCGAGGCCCACACCGACGTGTACCACAACCTGGACGA | KILRSLEGSDDAVLLQ |
| | | | GAACTCCCAGAAGATCCTGCGGAGCCTGGAAGGCAGTGATGATGCTG | RRLDNMNFKWSELRK |
| | | | TGCTCCTCCAGCGGAGACTGGACAACATGAACTTCAAGTGGAGCGAG | KSLNIRSHLEASSDQW |
| | | | CTGCGCAAGAAGTCCCTGAACATCAGAAGCCACCTGGAAGCCAGCAG | KRLHLSLQELLVWLQL |
| | | | CGACCAGTGGAAAAGACTGCATCTGTCCCTGCAAGAACTGCTCGTGT | KDDELSRQAPIGGDFP |
| | | | GGCTGCAGCTCAAGGACGACGAACTGAGCAGACAGGCCCCTATCGGC | AVQKQNDVHRAFKRE |
| | | | GGAGATTTTCCCGCCGTGCAGAAACAGAACGACGTGCACAGAGCCTT | LKTKEPVIMSTLETVRI |
| | | | CAAGCGCGAGCTGAAAACAAAAGAACCCGTCATCATGAGCACCCTGG | FLTEQPLEGLEKLYQEP |
| | | | AAACCGTGCGGATCTTTCTGACCGAGCAGCCCCTGGAAGGACTCGAG | RELPPEERAQNVTRLL |
| | | | AAGCTGTACCAAGAGCCTCGCGAACTGCCTCCTGAAGAAAGGGCCCA | RKQAEEVNTEWEKLN |
| | | | GAACGTGACCCGGCTGCTGAGAAAACAGGCCGAGGAAGTGAACACC | LHSADWQRKIDETLER |
| | | | GAGTGGGAAAAGCTGAATCTGCACTCCGCCGACTGGCAGCGGAAGAT | LRELQEATDELDLKLR |
| | | | CGATGAGACACTGGAACGGCTGCGGGAATTGCAAGAGGCCACCGAT | QAEVIKGSWQPVGDLL |
| | | | GAGCTGGACCTGAAGCTGAGACAGGCCGAAGTCATCAAAGGATCCTG | IDSLQDHLEKVKALRG |
| | | | GCAGCCTGTGGGCGACCTGCTCATCGATAGTCTGCAGGACCACCTCG | EIAPLKENVSHVNDLA |
| | | | AGAAAGTCAAAGCCCTGCGGGGAGAGATCGCCCCACTGAAAGAAAA | RQLTTLGIQLSPYNLST |
| | | | CGTGTCCCACGTGAACGACCTGGCCAGACAGCTGACAACACTGGGCA | LEDLNTRWKLLQVAV |
| | | | TCCAGCTGAGCCCCTACAACCTGTCCACACTTGAGGACCTGAACACCC | EDRVRQLHEAHRDFGP |
| | | | GGTGGAAACTGCTCCAAGTGGCCGTGGAAGATAGAGTGCGGCAGCTG | ASQHELSTSVQGPWER |
| | | | CACGAAGCCCACAGAGATTTTGGACCAGCCAGCCAGCACTTCCTGAG | AISPNKVPYYINHETQT |
| | | | CACCTCAGTTCAAGGCCCCTGGGAGAGAGCTATCAGCCCTAACAAGG | TCWDHPKMTELYQSL |
| | | | TGCCCTACTACATCAACCACGAGACACAGACCACCTGTTGGGATCAC | ADLNNVRFSAYRTAM |
| | | | CCCAAGATGACCGAGCTGTATCAGAGTCTGGCTGATCTGAACAATGT | KLRRLQKALCLDLLSL |
| | | | GCGGTTCAGCGCCTACCGGACCGCTATGAAGCTGCGAAGGCTGCAGA | SAACDALDQHNLKQN |
| | | | AAGCTCTGTGCCTGGACCTCCTGTCTCTGAGCGCTGCTTGTGATGCCC | DQPMDILQIINCLTTIY |
| | | | TGGACCAGCATAATCTGAAGCAGAACGACCAGCCTATGGACATCCTC | DRLEQEHNNLVNVPLC |
| | | | CAGATCATCAATTGCCTGACCACCATCTACGACCGCCTGGAACAAGA | VDMCLNWLLNVYDTG |
| | | | ACACAACAACCTCGTGAACGTGCCCCTGTGCGTGGACATGTGCCTGA | RTGRIRVLSFKTGIISLC |
| | | | ATTGGCTGCTGAATGTGTACGACACCGGCCGCACAGGCAGAATCAGA | KAHLEDKYRYLFKQV |
| | | | GTGCTGTCCTTCAAGACCGGCATCATCTCCCTGTGCAAGGCCCACCTC | ASSTGFCDQRRLGLLL |
| | | | GAAGATAAGTACCGCTACCTGTTCAAACAGGTGGCCTCCAGCACCGG | HDSIQIPRQLGEVASFG |
| | | | CTTTTGCGACCAAAGAAGGCTGGGACTGCTCCTGCACGACAGCATTC | GSNIEPSVRSCFQFANN |
| | | | AGATCCCTAGACAGCTGGGCGAAGTGGCCTCCTTTGGCGGCTCTAAT | KPEIEAALFLDWMRLE |
| | | | ATCGAGCCTAGCGTGCGGAGCTGCTTCCAGTTCGCCAACAACAAGCC | PQSMVWLPVLHRVAA |
| | | | TGAGATCGAGGCTGCCCTGTTCCTGGACTGGATGAGACTCGAGCCTC | AETAKHQAKCNICKEC |
| | | | AGAGCATGGTCTGGCTGCCAGTGCTGCATAGAGTGGCCGCTGCCGAA | PIIGFRYRSLKHFNYDIC |
| | | | ACAGCCAAGCACCAGGCCAAGTGCAACATCTGCAAAGAGTGCCCCAT | QSCFFSGRVAKGHKM |
| | | | CATCGGCTTCCGGTACAGATCTCTGAAGCACTTCAACTACGATATCTG | HYPMVEYC (SEQ ID |
| | | | CCAGAGCTGTTTCTTCAGCGGCAGAGTCGCCAAGGGCCACAAAATGC | NO: 30) |
| | | | ACTACCCCATGGTGGAATACTGC (SEQ ID NO: 29) | |

WT = wild type; CO = codon optimized; H = hinge; R = spectrin repeat; CT = C-terminus; A = deleted region

What is claimed is:

1. A pharmaceutical composition comprising:
a. a circular RNA polynucleotide encoding a muscle protein or protein complex, or a variant thereof, and
b. a transfer vehicle comprising an ionizable lipid.

2. The pharmaceutical composition of claim 1, wherein the ionizable lipid is a compound of Formula (1): 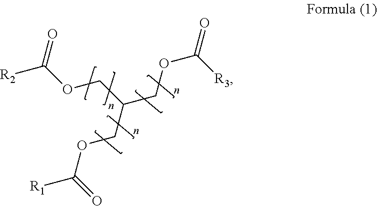
or a pharmaceutically acceptable salt thereof, wherein:
each n is independently an integer from 1-15;
R₁ and R₂ are each independently selected from a group consisting of:

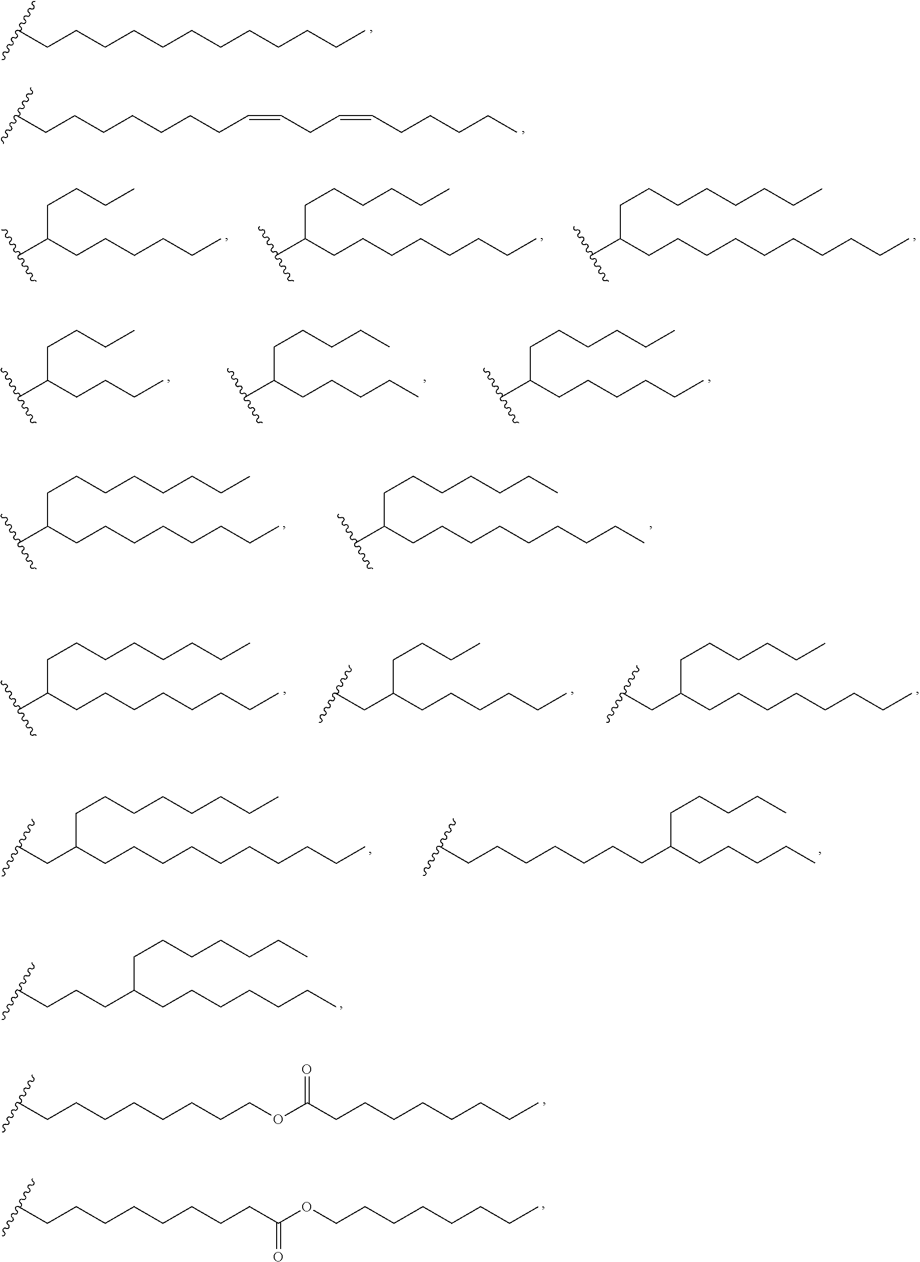

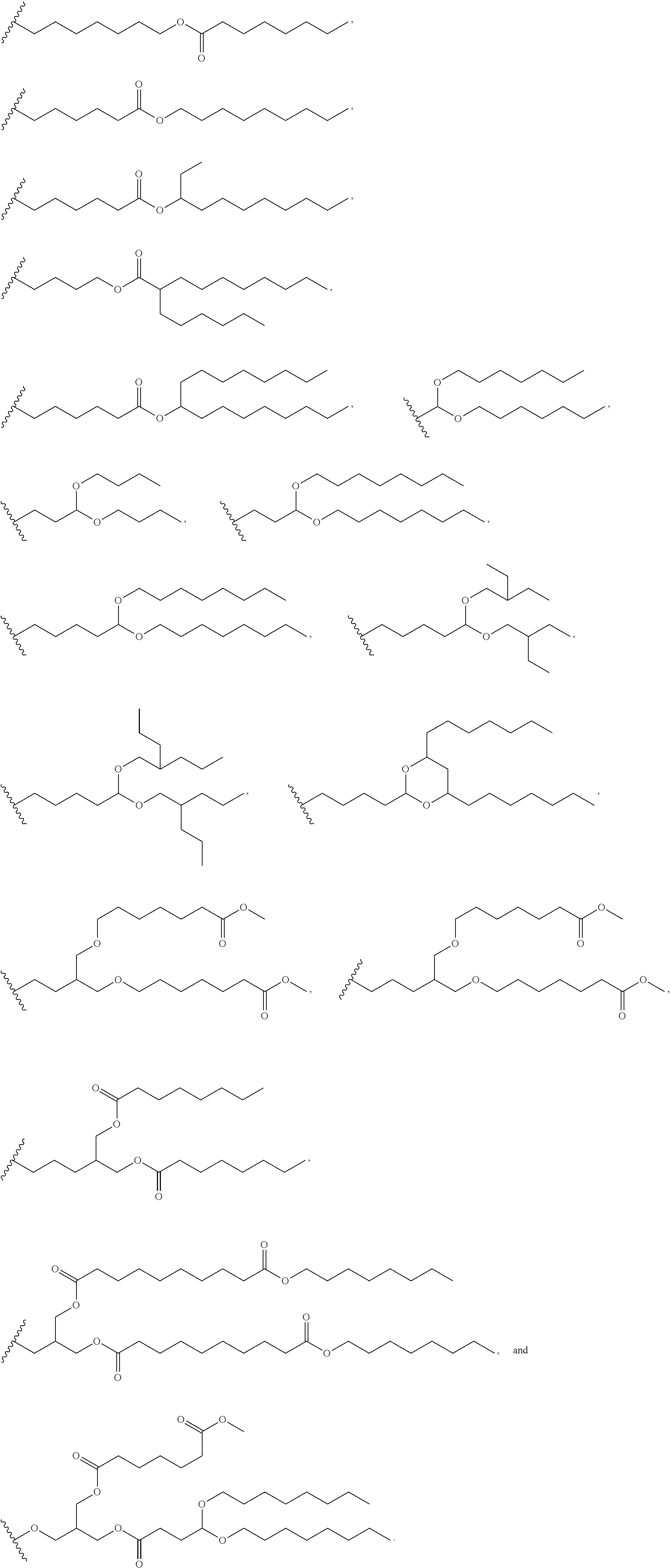

3. The pharmaceutical composition of claim 2, wherein Ra is selected from a group consisting of:

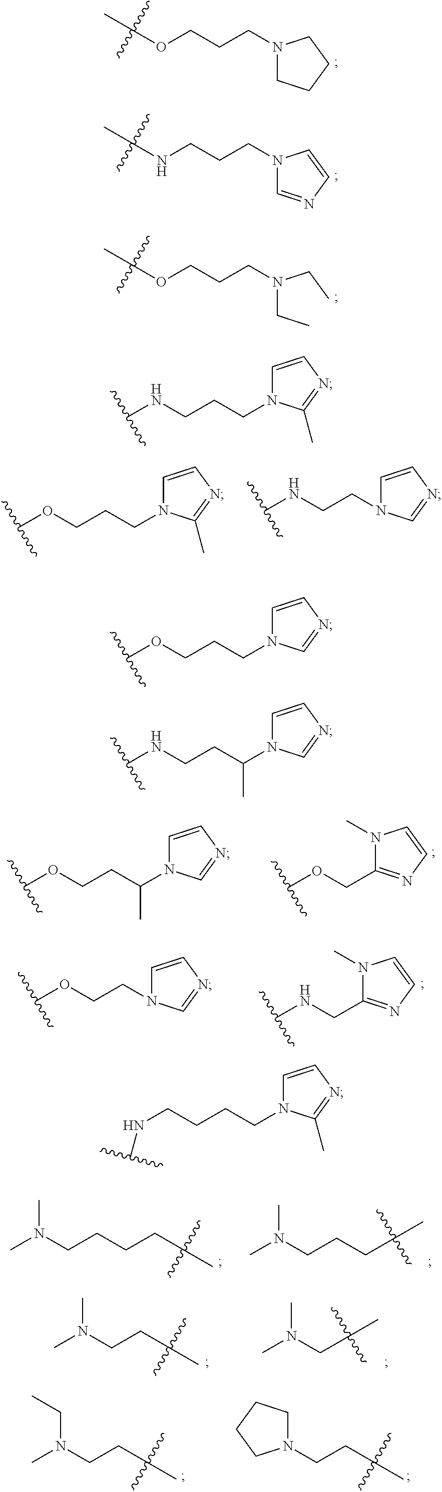

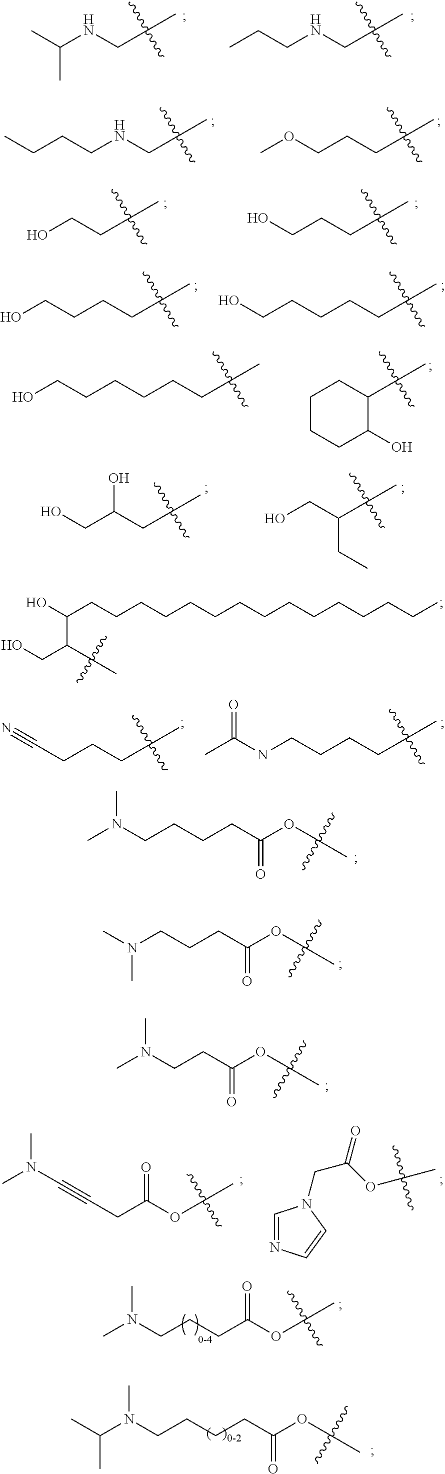

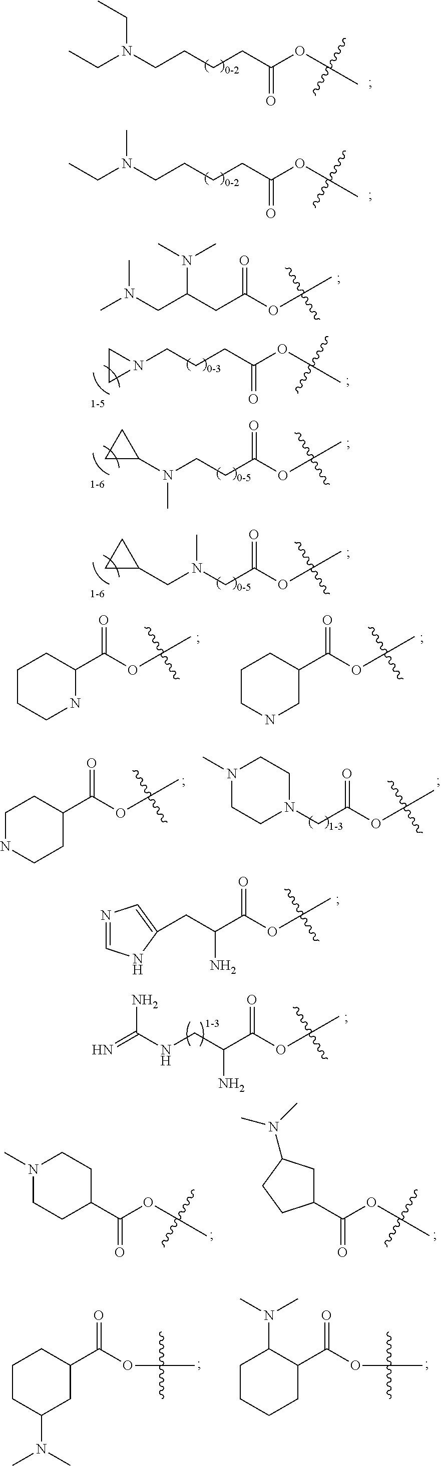

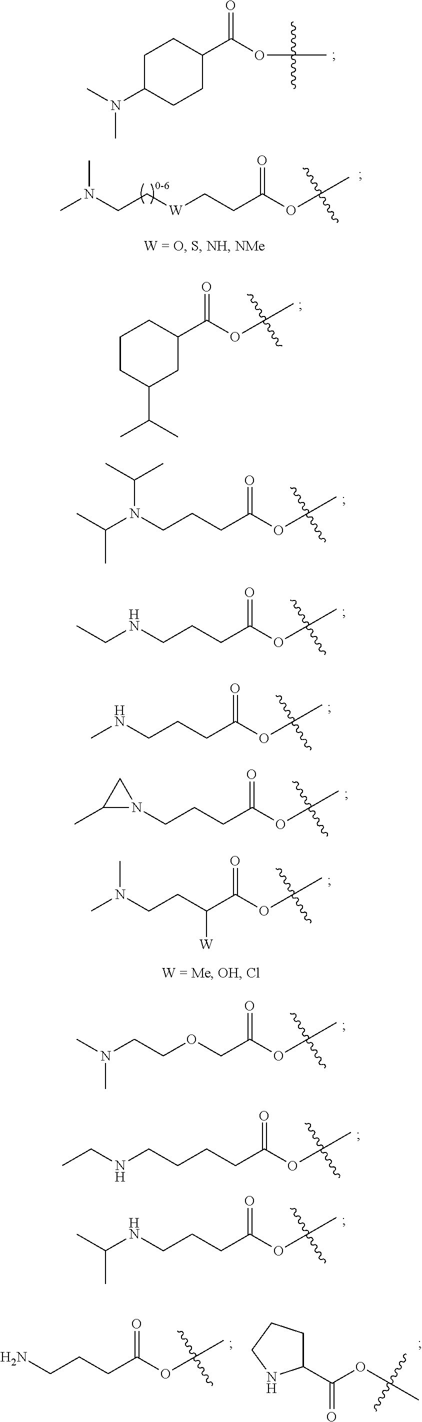

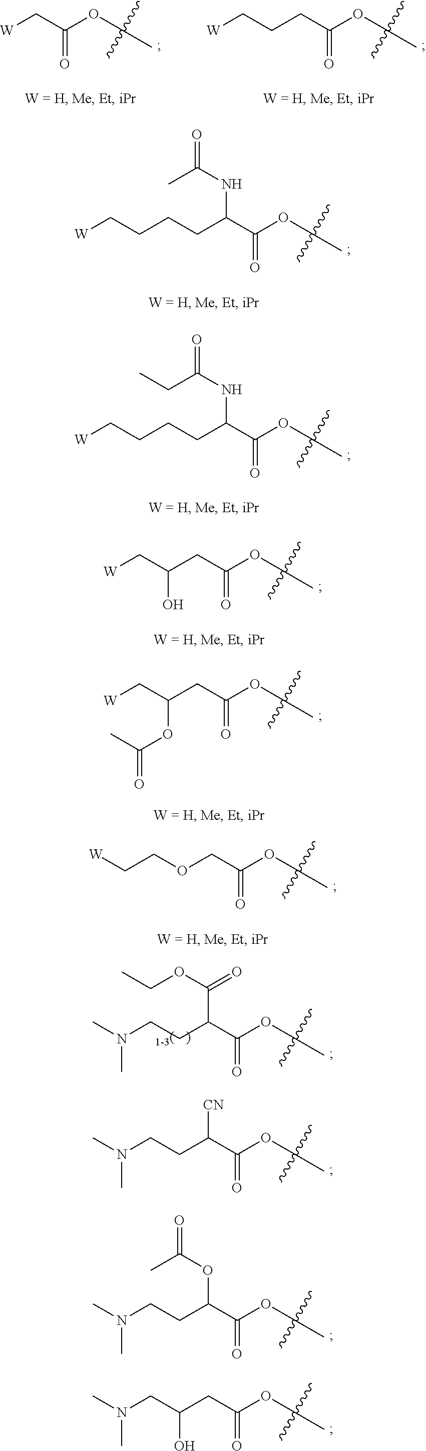

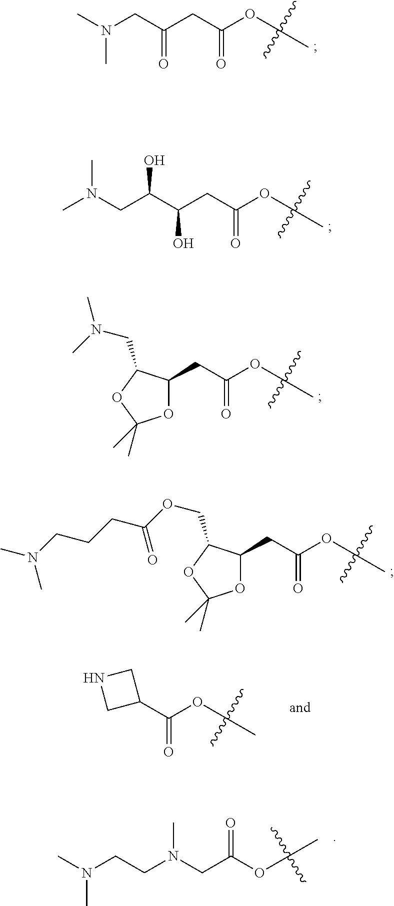 [SMILES: *OC(=O)CC(=O)CN(C)C ; *OC(=O)C[C@@H](O)[C@H](O)CN(C)C ; *OC(=O)C[C@H]1OC(C)(C)O[C@@H]1CN(C)C ; *OC(=O)C[C@H]1OC(C)(C)O[C@@H]1COC(=O)CCCN(C)C ; *OC(=O)C1CNC1 ; *OC(=O)CN(C)CCN(C)C]

4. The pharmaceutical composition of any one of claims 1-3, wherein the ionizable lipid is represented by:

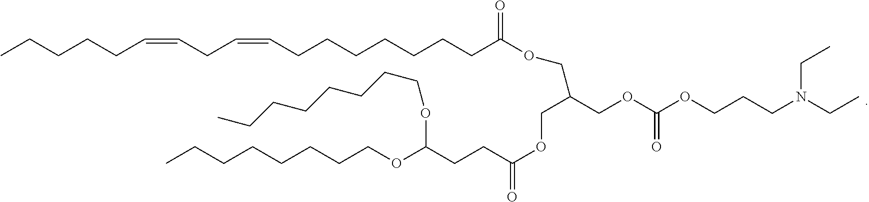 [SMILES: CCCCC/C=C\C/C=C\CCCCCCCC(=O)OCC(COC(=O)CCC(OCCCCCCCC)OCCCCCCCC)COC(=O)OCCCN(CC)CC]

5. The pharmaceutical composition of any one of claims 1-4, wherein the circular RNA polynucleotide comprises a core functional element comprising, in the following order:
(i) a translation initiation element (TIE), and
(ii) a coding element encoding for the muscle protein or protein complex.

6. The pharmaceutical composition of any one of claims 1-5, wherein the muscle protein or protein complex is a human or humanized muscle protein or protein complex.

7. The pharmaceutical composition of any one of claims 1-6, wherein the muscle protein or protein complex is a smooth, skeletal, or cardiac muscle protein or protein complex.

8. The pharmaceutical composition of claim 7, wherein the muscle protein or protein complex is a smooth muscle myosin, actin, tropomyosin, calponin, or caldesmon, or a fragment, isoform, or variant thereof.

9. The pharmaceutical composition of claim 7, wherein the muscle protein or protein complex is a skeletal or muscular sarcolemmal protein, laminin, dystroglycan, collagen, actin, myosin, myofibrillar protein, dystrophin, or intermediate protein, or a fragment, isoform, or variant thereof.

10. The pharmaceutical composition of claim 9, wherein the muscle protein or protein complex is dystrophin or a fragment, isoform, or variant thereof.

11. The pharmaceutical composition of claim 10, wherein the dystrophin variant comprises a mutation, truncation, deletion, or insertion of a naturally occurring or synthetic dystrophin.

12. The pharmaceutical composition of claim 10 or 11, wherein the dystrophin variant is at least about 1900 amino acids in length.

13. The pharmaceutical composition of any one of claims 10-12, wherein the dystrophin variant comprises a dystrophin central rod domain in whole or in part from a human full-length dystrophin.

14. The pharmaceutical composition of any one of claims 10-13, wherein the dystrophin variant lacks a C-terminal domain.

15. The pharmaceutical composition of any one of claims 10-14, wherein the dystrophin variant comprises about 6-24 spectrin-like repeats.

16. The pharmaceutical composition of any one of claims 10-15, wherein the dystrophin variant further comprises a synthrophin-binding domain.

17. The pharmaceutical composition of any one of claims 10-16, wherein the dystrophin variant comprises a hinge domain.

18. The pharmaceutical composition of claim 17, wherein the dystrophin variant comprises a hinge 1, hinge 2, hinge 3 or hinge 4 domain.

19. The pharmaceutical composition of any one of claims 10-18, wherein the dystrophin variant comprises a Becker variant.

20. The pharmaceutical composition of claim 10, wherein the dystrophin or fragment, isoform, or variant thereof comprises a full-length naturally occurring or synthetic dystrophin.

21. The pharmaceutical composition of claim 20, wherein the dystrophin or fragment, isoform, or variant thereof comprises at least 3500 amino acids in length.

22. The pharmaceutical composition of any one of claims 10-21, wherein the dystrophin or fragment, isoform, or variant thereof comprises or consists of a sequence selected from Table 1.

23. The pharmaceutical composition of any one of claims 1-7, wherein the muscle protein or protein complex is a dystrophin-associated protein or a fragment thereof.

24. The pharmaceutical composition of claim 23, wherein the dystrophin-associated protein comprises a α-dystroglycan, β-dystroglycan, sarcoglycans, sarcospan, dystrobrevin, syntrophins, neuronal nitric oxide synthase or fragment thereof.

25. The pharmaceutical composition of any one of claims 5-7, wherein the coding element comprises an intein-containing split protein gene.

26. The pharmaceutical composition of claim 25, wherein the intein-containing split protein gene comprises a split dystrophin gene.

27. The pharmaceutical composition of any one of claims 5-26, wherein the TIE comprises an untranslated region (UTR) or a fragment thereof, an aptamer complex or a fragment thereof, or a combination thereof.

28. The pharmaceutical composition of claim 27, wherein the UTR or fragment thereof is derived from a viral or eukaryotic messenger RNA.

29. The pharmaceutical composition of claim 28, wherein the UTR or fragment thereof comprises a viral or eukaryotic messenger RNA.

30. The pharmaceutical composition of claim 29, wherein the UTR or fragment thereof comprises a viral internal ribosome entry site (IRES) or eukaryotic IRES.

31. The pharmaceutical composition of claim 30, wherein the IRES comprises one or more modified nucleotides compared to the wild-type viral IRES or eukaryotic IRES.

32. The pharmaceutical composition of claim 27, wherein the aptamer complex or fragment thereof comprises a natural or synthetic aptamer sequence.

33. The pharmaceutical composition of claim 27, wherein the aptamer complex or a fragment thereof comprises more than one aptamer.

34. The pharmaceutical composition of any one of claims 27-33, wherein the TIE comprises a UTR and an aptamer complex.

35. The pharmaceutical composition of claim 34, wherein the UTR is located upstream to the aptamer complex.

36. The pharmaceutical composition of any one of claims 5-35, wherein the TIE comprises an accessory element.

37. The pharmaceutical composition of claim 36, wherein the accessory element comprises a miRNA binding site or a fragment thereof, a restriction site or a fragment thereof, an RNA editing motif or a fragment thereof, a zip code element or a fragment thereof, an RNA trafficking element or a fragment thereof, or a combination thereof.

38. The pharmaceutical composition of claim 37, wherein the accessory element comprises a binding domain to an IRES transacting factor (ITAF).

39. The pharmaceutical composition of claim 38, wherein the binding domain comprises a polyA region, a polyC region, a poly AC region, a polyprimidine tract, or a combination or variant thereof.

40. The pharmaceutical composition of claim 38, wherein the ITAF comprises a poly (rC)-binding protein 1 (PCBP1), PCBP2, PCBP3, PCBP4, poly (A)-binding protein 1 (PABP1), polyprimidine-tract binding protein (PTB), Argonaute protein family member, HNRNPK (heterogeneous nuclear ribonucleoprotein K protein), or La protein, or a fragment or combination thereof.

41. The pharmaceutical composition of any one of claims 5-40, wherein the core functional element comprises a termination element.

42. The pharmaceutical composition of any one of claims 1-41, having an in vivo duration of therapeutic effect in humans of at least 20 hours.

43. The pharmaceutical composition of any one of claims 1-42, having a functional half-life of at least 6 hours.

44. The pharmaceutical composition of any one of claims 1-43, wherein the circular RNA polynucleotide comprises a spacer sequence.

45. The pharmaceutical composition of any one of claims 1-44, wherein the circular RNA polynucleotide comprises two or more duplex forming sequences.

46. The pharmaceutical composition of any one of claims 1-45, wherein the circular RNA polynucleotide comprises an exon element.

47. The pharmaceutical composition of claim 46, wherein the circular RNA polynucleotide comprises a 5′ exon element and a 3′ exon element.

48. A precursor RNA polynucleotide of the circular RNA polynucleotide comprised in the pharmaceutical composition of any of claims 1-47.

49. The precursor RNA polynucleotide of claim 48, comprising two or more expression sequences encoding for the muscle protein or protein complex.

50. The precursor RNA polynucleotide of claim 49, comprising a polynucleotide sequence encoding a proteolytic cleavage site or a ribosomal stuttering element between the first and second expression sequence.

51. The precursor RNA polynucleotide of claim 50, wherein the ribosomal stuttering element is a self-cleaving spacer.

52. The precursor RNA polynucleotide of claim 50, comprising a polynucleotide sequence encoding 2A ribosomal stuttering peptide.

53. The precursor RNA polynucleotide of claim 48, comprising two or more internal ribosome entry sites (IRESs).

54. The precursor RNA polynucleotide of claim 48, comprising a first TIE, a first coding element, a first termination sequence, optionally a spacer, a second TIE, a second coding element, and second a termination sequence, wherein the first TIE and the second TIE each comprises an IRES.

55. The precursor RNA polynucleotide of any one of claims 48-54, wherein the precursor RNA comprises an intron element.

56. The precursor RNA polynucleotide of claim 55, wherein the precursor RNA comprises a 5′ intron element and a 3′ intron element.

57. The precursor RNA polynucleotide of any one of claims 48-56, wherein the precursor RNA comprises a spacer sequence.

58. The precursor RNA polynucleotide of any one of claims 48-57, wherein the precursor RNA comprises an affinity sequence or a leading untranslated sequence.

59. The precursor RNA polynucleotide of any one of claims 48-58, wherein the precursor RNA comprises a 5′ duplex sequence and a 3′ duplex sequence.

60. The precursor RNA polynucleotide of any one of claims 48-59, wherein the precursor RNA is transcribed from a vector or DNA comprising a PCR product, a linearized plasmid, non-linearized plasmid, linearized minicircle, a non-linearized minicircle, viral vector, cosmid, ceDNA, or an artificial chromosome.

61. The pharmaceutical composition of any one of claims 1-47, wherein the transfer vehicle comprises a nanoparticle.

62. The pharmaceutical composition of claim 61, wherein the nanoparticle is a lipid nanoparticle, a core-shell nanoparticle, or a biodegradable nanoparticle.

63. The pharmaceutical composition of claim 61or 62, wherein the nanoparticle comprises one or more cationic lipids, ionizable lipids, or poly β-amino esters.

64. The pharmaceutical composition of any one of claims 61-63, wherein the nanoparticle comprises a DSPE, DOPE, or a combination thereof.

65. The pharmaceutical composition of any one of claims 61-64, wherein the nanoparticle comprises one or more non-cationic lipids.

66. The pharmaceutical composition of any one of claims 61-65, wherein the nanoparticle comprises one or more PEG-modified lipids, polyglutamic acid lipids, or hyaluronic acid lipids.

67. The pharmaceutical composition of any one of claims 61-66, wherein the nanoparticle comprises cholesterol.

68. The pharmaceutical composition of any one of claims 61-67, wherein the nanoparticle comprises arachidonic acid, leukotriene, or oleic acid.

69. A method of producing muscle protein or protein complex in a muscle cell or muscle tissue, comprising delivering the pharmaceutical composition of any one of claims 1-47 using systemic administration.

70. The method of claim 69, wherein the systemic administration comprises intravenous (i.v.) injection.

71. The method of claim 69 or 70, wherein the muscle tissue is cardiac or diaphragm muscle tissue.

72. A method of treating a subject in need thereof comprising administering a therapeutically effective amount of a pharmaceutical composition of any one of claims 1-47.

73. The method of claim 72, wherein the subject has muscular dystrophy, dystroglycanopathy, collagen VI myopathy, Limb-girdle muscular dystrophies (LGMD), myofibrillar myopathy, or dilated cardiomyopathy.

74. The method of claim 73, wherein the subject has Becker's disease, Duchenne Muscular Dystrophy (DMD), or Congenital Muscular Dystrophy type 1A (MDCIA), Ullrich congenital muscular dystrophy (UCMD), Walker-Warburg syndrome, Muscle-eye-brain syndrome or Bethlem myopathy (BM).

75. The method of any one of claims 72-74, wherein the therapeutically effective amount is less than or equal to about 3 mg/kg of circular RNA polynucleotide.

76. The method of claim 75, wherein the therapeutically effective amount is less than or equal to about 1 mg/kg of circular RNA polynucleotide.
